(19) United States
(12) Patent Application Publication
(10) Pub. No.: US 2016/0259881 A1
(43) Pub. Date: Sep. 8, 2016

(54) METHODS OF INCORPORATION OF TRANSCRIPT CHROMOSOMAL LOCUS INFORMATION FOR IDENTIFICATION OF BIOMARKERS OF DISEASE RECURRENCE RISK

(71) Applicant: GENOMIC HEALTH, INC., Redwood City, CA (US)

(72) Inventors: Michael C. Kiefer, Walnut Creek, CA (US); Francois Collin, Berkeley, CA (US); Samuel Levy, La Jolla, CA (US); Joffre B. Baker, Redwood City, CA (US); Michael R. Crager, Redwood City, CA (US); Mark R. Segal, Redwood CIty, CA (US)

(21) Appl. No.: 15/033,055

(22) PCT Filed: Oct. 28, 2014
(86) PCT No.: PCT/US14/62715

§ 371 Date: Apr. 28, 2016

Related U.S. Application Data
(60) Provisional application No. 61/897,059, filed on Oct. 29, 2013.

(51) Int. Cl. G06F 19/18; G06F 19/26; C12Q 1/68; G06F 19/20

(52) U.S. Cl. CPC G06F 19/18; G06F 19/20; G06F 19/26; C12Q 1/6886; C12Q 2600/158

(57) ABSTRACT

The present invention provides methods for incorporating transcript chromosomal locus information for identification of biomarkers of disease recurrence risk.

### RELATED APPLICATION

This application claims the benefit of priority of U.S. Provisional Application No. 61/897,059, filed Oct. 29, 2013, all of which is hereby incorporated by reference in its entirety.

### TECHNICAL FIELD

The present invention relates to methods of combining chromosomal site of transcription information with RNA expression for clinical biomarker discovery. The identified biomarkers include coding transcripts and their expression products, as well as non-coding transcripts, and are useful for predicting the likelihood of breast cancer recurrence in a breast cancer patient

### INTRODUCTION

Tumor levels of certain individual RNA species serve as clinically useful prognostic (Paik S, et al. (2004). N Engl J Med 351: 2817-2826; Habel L A, et al. (2006). Breast Cancer Res 8: R25; Van't Veer L J, et al. (2002) Nature 415: 530-536) and predictive (Paik S, et al. (2006). J Clin Oncol 24: 3726-3734; Gianni L, et al. (2005) J Clin Oncol 23: 7265-7277) biomarkers in several cancers. Discovery and development of these RNA biomarkers has been based on use of RT-PCR or DNA microarrays to screen hundreds to thousands of tumor tissue mRNAs to identify relatively small subsets of mRNAs that repeatedly associate with disease outcome in multiple patient cohorts. The most useful tests are based on building multi-gene classifiers that incorporate on the order of a dozen to several dozen different mRNA species. Widespread clinical adoption requires that prospective tests be validated in one or more clinical studies.

RNA-Seq represents a technology advance over RT-PCR and DNA microarrays for initial discovery of RNAs that associate with clinical outcomes (Tucker et al., The American J. Human Genetics 85:142-154, 2009; Sinicropi et al. PLoS One. 7:e4009, 2012; Levin et al. Genome Biology 2009, 10:R115), due to the combined sensitivity, precision and high throughput of massively parallel sequencing. For example, we recently reported the discovery of more than a thousand mRNA species that associate with risk of recurrence of breast cancer, based on whole transcriptome RNA-Seq analysis of tumors from 136 early breast cancer patients (“Providence patient cohort”). We showed a high level of concordance between measured RNA levels derived from RNA-Seq and RT-PCR, which has been considered the gold standard for quantification of RNAs (M. Cronin, et al., Am J Pathol 164:35-42, 2004). In addition, whole transcriptome RNA-Seq quantifies greater numbers of mRNA species than DNA microarrays (heretofore the platform capable of evaluating the largest numbers of RNA species), notably intronic and intragenic RNA species. It is noteworthy that in this RNA-Seq study of the Providence cohort a greater number of intronic versus exonic RNA species associate with recurrence risk (Sinicropi et al. PLoS One. 7:e4009, 2012).

RNA-Seq and DNA microarray capabilities to evaluate thousands of different RNA species are useful for biomarker discovery, but their utility can be diminished due to the large numbers of RNAs that have little or no association with clinical outcome, decreasing the statistical power of false discovery rate (FDR) controlling analyses to identify biomarkers (Crager, Genetic Epidemiology 36:839-847, 2012). This problem is compounded because typically the sizes of available patient cohorts are no more than several hundred patients. The identification power can be improved by prospectively nominating classes of RNAs considered to be most promising, based on biological or biomedical prior knowledge, and carrying out a separate class analysis (Efron B Annals of Applied Statistics 2:197-223, 2008; Crager, Genetic Epidemiology 36:839-847, 2012).

### SUMMARY

This application discloses methods for identifying prognostic features based on RNAs or proteins. The method can be used to analyze RNAs or proteins in various diseases, including cancer and non-cancer-based diseases. The disclosed methods can use RNA-Seq, RT-PCR, expression arrays, and/or proteomic methods, such as Western blots and mass spectrometry.

In the one approach (termed “mapped strings”), the expression of a population of RNAs can be evaluated to identify individual RNA species associated with breast recurrence rate, as is conventional practice. Each member of the subset of genes that is identified at a given false discovery rate as associating with recurrence risk is then graphically placed on its chromosomal locus. Remarkably, this demonstrates that genes associated with good prognosis tend to distribute in long strings uninterrupted by genes associated with bad prognosis, and the reverse is true for genes associated with bad prognosis. Each identified string is then evaluated as a unit (“metagene”) candidate biomarker for risk of disease recurrence.

In the second approach the average measured normalized quantity of each transcript species is calculated for the entire patient cohort being studied, standardizing the level of each species to produce a centered scaled robust z-score. This is used to construct for each patient a transcriptome profile physical map (TPM) where the X-axis represents the chromosomal locus for each RNA species and the Y-axis represents, for each RNA species, the individual patient's standardized normalized abundance value. These graphs reveal striking patient to patient differences, for example various sizes and numbers of spike-like features (segments) and global clouds of data points that vary considerably in their dispersion. These features, or summaries thereof, are then evaluated as candidate biomarkers for disease recurrence.

Data in this application demonstrate that these approaches yield novel breast cancer recurrence risk markers that prove to be stably prognostic when tested across breast cancer patient cohorts/datasets.

### BRIEF DESCRIPTION OF THE DRAWINGS

FIG. 1A places the identified 1307 genes identified from the Providence dataset on the physical map of the human genome, demonstrating that there are physical gaps, and often large ones, between nearest neighbors in a string. FIG. 1B shows gross spatial chromosomal arrangement of the 363 prognostic genes (ER+ patients only) from the Providence dataset.

FIG. 2 shows 2032 genes on their chromosomal coordinates, revealing many long strings of identified genes from NKI DNA-microarray gene expression data. (Data obtained from van't Veer L J et al. Nature 415:530-6, 2002 website).

FIG. 3A shows percentage of individual genes in each string with prognostic value less than the string for both ER+ and ER− samples (Providence data). FIG. 3B shows percentage of individual genes in each string with prognostic value less than the string for ER+ samples (Providence data).

FIG. 4A-E show transcriptome profile maps (TPM) for 5 different Providence patient tumors.

FIG. 5A-D show TPM segment plots derived from Providence RNA-Seq data.

FIG. 6A-E show TPMs from NM cohort breast cancer data.

### DEFINITIONS

Before the present invention and specific exemplary embodiments of the invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

As used herein and in the appended claims, the singular forms “a,” “an,” and “the” include plural referents unless the context clearly dictates otherwise. Thus, for example, reference to “an RNA transcript” includes a plurality of such RNA transcripts.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

Additionally, the practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

One skilled in the art will recognize many methods and materials similar or equivalent to those described herein, which could be used in the practice of the present invention. Indeed, the present invention is in no way limited to the methods and materials described herein. For purposes of the invention, the following terms are defined below.

The terms “tumor” and “lesion” as used herein, refer to all neoplastic cell growth and proliferation, whether malignant or benign, and all pre-cancerous and cancerous cells and tissues. Those skilled in the art will realize that a tumor tissue sample may comprise multiple biological elements, such as one or more cancer cells, partial or fragmented cells, tumors in various stages, surrounding histologically normal-appearing tissue, and/or macro or micro-dissected tissue.

The terms “cancer” and “cancerous” refer to or describe the physiological condition in mammals that is typically characterized by unregulated cell growth. Examples of cancer in the present disclosure include breast cancer, prostate cancer, colon cancer, bladder cancer, melanoma, leukemias, etc.

As used herein, the term “breast cancer” is used in the broadest sense and refers to all stages and all forms of cancer arising from the tissue of the breast.

As used herein, the term “exon” refers to any segment of an interrupted gene that is represented in the mature RNA product (B. Lewin. Genes IV Cell Press, Cambridge Mass. 1990). As used herein, the terms “intron” and “intronic sequence” refer to any non-coding region found within genes.

The term “expression product” as used herein refers to an expression product of a coding RNA transcript. Thus, the term refers to a polypeptide or protein.

As used herein, the term “intergenic region” refers to a stretch of DNA or RNA sequences located between clusters of genes that contain few or no genes. Intergenic regions are different from intragenic regions (or “introns”), which are non-coding regions that are found between exons within genes. An intergenic region may be comprised of one or more “intergenic sequences.”

As used herein, the terms “long intergenic non-coding RNAs” and “lincRNAs” are used interchangeably and refer to non-coding transcripts that are typically longer than 200 nucleotides.

As used herein, the term “level” as used herein refers to qualitative or quantitative determination of the number of copies of a coding or non-coding RNA transcript or a polypeptide/protein. An RNA transcript or a polypeptide/protein exhibits an “increased level” when the level of the RNA transcript or polypeptide/protein is higher in a first sample, such as in a clinically relevant subpopulation of patients (e.g., patients who have experienced cancer recurrence), than in a second sample, such as in a related subpopulation (e.g., patients who did not experience cancer recurrence). In the context of an analysis of a level of an RNA transcript or a polypeptide/protein in a tumor sample obtained from an individual patient, an RNA transcript or polypeptide/protein exhibits “increased level” when the level of the RNA transcript or polypeptide/protein in the subject trends toward, or more closely approximates, the level characteristic of a clinically relevant subpopulation of patients.

Thus, for example, when the RNA transcript analyzed is an RNA transcript that shows an increased level in subjects that experienced long-term survival without cancer recurrence as compared to subjects that did not experience long-term survival without cancer recurrence, then an “increased” level of a given RNA transcript can be described as being positively correlated with a likelihood of long-term survival without cancer recurrence. If the level of the RNA transcript in an individual patient being assessed trends toward a level characteristic of a subject who experienced long-term survival without cancer recurrence, the level of the RNA transcript supports a determination that the individual patient is more likely to experience long-term survival without cancer recurrence. If the level of the RNA transcript in the individual patient trends toward a level characteristic of a subject who experienced cancer recurrence, then the level of the RNA transcript supports a determination that the individual patient is more likely to experience cancer recurrence.

The term “likelihood score” is an arithmetically or mathematically calculated numerical value for aiding in simplifying or disclosing or informing the analysis of more complex quantitative information, such as the correlation of certain levels of the disclosed RNA transcripts, their expression products, or gene networks to a likelihood of a certain clinical outcome in a breast cancer patient, such as likelihood of long-term survival without breast cancer recurrence. A likelihood score may be determined by the application of a specific algorithm. The algorithm used to calculate the likelihood score may group the RNA transcripts, or their expression products, into gene networks. A likelihood score may be determined for a gene network by determining the level of one or more RNA transcripts, or an expression product thereof, and weighting their contributions to a certain clinical outcome such as recurrence. A likelihood score may also be determined for a patient. In an embodiment, a likelihood score is a recurrence score, wherein an increase in the recurrence score negatively correlates with an increased likelihood of long-term survival without breast cancer recurrence. In other words, an increase in the recurrence score correlates with bad prognosis. Examples of methods for determining the likelihood score or recurrence score are disclosed in U.S. Pat. No. 7,526,387.

The term “long-term” survival as used herein refers to survival for at least 3 years. In other embodiments, it may refer to survival for at least 5 years, or for at least 10 years following surgery or other treatment.

As used herein, the term “normalized” with regard to a coding or non-coding RNA transcript, or an expression product of the coding RNA transcript, refers to the level of the RNA transcript, or its expression product, relative to the mean levels of transcript/product of a set of reference RNA transcripts, or their expression products. The reference RNA transcripts, or their expression products, are based on their minimal variation across patients, tissues, or treatments. Alternatively, the coding or non-coding RNA transcript, or its expression product, may be normalized to the totality of tested RNA transcripts, or a subset of such tested RNA transcripts.

As used herein, the term “pathology” of cancer includes all phenomena that comprise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes.

A “patient response” may be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the cancer; (8) increase in the length of survival following treatment; and/or (9) decreased mortality at a given point of time following treatment.

The term “prognosis” as used herein, refers to the prediction of the likelihood of cancer-attributable death or progression, including recurrence, metastatic spread, and drug resistance, of neoplastic disease, such as breast cancer. The term “prediction” is used herein to refer to the likelihood that a patient will respond either favorably or unfavorably to a drug or set of drugs, and also the extent of those responses, or that a patient will survive, following surgical removal of the primary tumor and/or chemotherapy for a certain period of time without cancer recurrence. The methods of the present invention can be used clinically to make treatment decisions by choosing the most appropriate treatment modalities for any particular patient. The methods of the present invention are tools in predicting if a patient is likely to respond favorably to a treatment regimen, such as surgical intervention, chemotherapy with a given drug or drug combination, and/or radiation therapy, or whether long-term survival of the patient without cancer recurrence is likely, following surgery and/or termination of chemotherapy or other treatment modalities.

The term “breast cancer prognostic biomarker” refers to an RNA transcript, or an expression product thereof, intronic RNA, lincRNA, intergenic sequence, and/or intergenic region found to be associated with long term survival without breast cancer recurrence.

The term “reference” RNA transcript or an expression product thereof, as used herein, refers to an RNA transcript or an expression product thereof, whose level can be used to compare the level of an RNA transcript or its expression product in a test sample. In an embodiment of the invention, reference RNA transcripts include housekeeping genes, such as beta-globin, alcohol dehydrogenase, or any other RNA transcript, the level or expression of which does not vary depending on the disease status of the cell containing the RNA transcript or its expression product. In another embodiment, all of the assayed RNA transcripts, or their expression products, or a subset thereof, may serve as reference RNA transcripts or reference RNA expression products.

As used herein, the term “RefSeq RNA” refers to an RNA that can be found in the Reference Sequence (RefSeq) database, a collection of publicly available nucleotide sequences and their protein products built by the National Center for Biotechnology Information (NCBI). The RefSeq database provides an annotated, non-redundant record for each natural biological molecule (i.e. DNA, RNA or protein) included in the database. Thus, a sequence of a RefSeq RNA is well-known and can be found in the RefSeq database at http://www.ncbi.nlm.nih.gov/RefSeq/. See also Pruitt et al., Nucl. Acids Res. 33(Supp 1):D501-D504 (2005).

As used herein, the term “RNA transcript” refers to the RNA transcription product of DNA and includes coding and non-coding RNA transcripts. RNA transcripts include, for example, mRNA, an unspliced RNA, a splice variant mRNA, a microRNA, fragmented RNA, long intergenic non-coding RNAs (lincRNAs), intergenic RNA sequences or regions, and intronic RNAs.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer (e.g., breast cancer), including those who have undergone or are candidates for resection (surgery) to remove cancerous tissue.

As used herein, the term “surgery” applies to surgical methods undertaken for removal of cancerous tissue, including mastectomy, lumpectomy, lymph node removal, sentinel lymph node dissection, prophylactic mastectomy, prophylactic ovary removal, cryotherapy, and tumor biopsy. The tumor samples used for the methods of the present invention may have been obtained from any of these methods.

The term “tumor” as used herein, refers to all neoplastic cell growth and proliferation, whether malignant or benign, and all pre-cancerous and cancerous cells and tissues.

The term “tumor sample” as used herein refers to a sample comprising tumor material obtained from a cancer patient. The term encompasses tumor tissue samples, for example, tissue obtained by surgical resection and tissue obtained by biopsy, such as for example, a core biopsy or a fine needle biopsy. In a particular embodiment, the tumor sample is a fixed, wax-embedded tissue sample, such as a formalin-fixed, paraffin-embedded tissue sample. Additionally, the term “tumor sample” encompasses a sample comprising tumor cells obtained from sites other than the primary tumor, e.g., circulating tumor cells. The term also encompasses cells that are the progeny of the patient's tumor cells, e.g. cell culture samples derived from primary tumor cells or circulating tumor cells. The term further encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a patient's tumor material.

As used herein, “whole transcriptome sequencing” refers to the use of high throughput sequencing technologies to sequence the entire transcriptome in order to get information about a sample's RNA content. Whole transcriptome sequencing can be done with a variety of platforms for example, the Genome Analyzer (Illumina, Inc., San Diego, Calif.), the SOLiD™ Sequencing System (Life Technologies, Carlsbad, Calif.), Ion Torrent (Life Technologies, Carlsbad, Calif.), and GS FLX and GS Junior Systems (454 Life Sciences, Roche, Branford, Conn.). However, any platform useful for whole transcriptome sequencing may be used.

The term “RNA-Seq” or “transcriptome sequencing” refers to sequencing performed on RNA (or cDNA) instead of DNA, where typically, the primary goal is to measure expression levels, detect fusion transcripts, alternative splicing, and other genomic alterations that can be better assessed from RNA. RNA-Seq includes whole transcriptome sequencing as well as target specific sequencing.

The term “computer-based system,” as used herein, refers to the hardware means, software means, and data storage means used to analyze information. The minimum hardware of a patient computer-based system comprises a central processing unit (CPU), input means, output means, and data storage means. A skilled artisan can readily appreciate that many of the currently available computer-based system are suitable for use in the present invention and may be programmed to perform the specific measurement and/or calculation functions of the present invention.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” or “computing means” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

A “mapped string” or “string” as used herein refers to the result of investigating the expression of a population of RNAs is evaluated to identify individual RNA species associated with disease recurrence rate, where each member of the subset of genes that is identified at a given false discovery rate as associating with recurrence risk is then graphically placed on its chromosomal locus. Mapped strings demonstrate that genes associated with good prognosis tend to distribute in long strings uninterrupted by genes associated with bad prognosis, and the reverse is true for genes associated with bad prognosis. Each identified string may then be evaluated as a unit (metagene) candidate biomarker for risk of disease recurrence.

A “transcriptome profile map” or “TPM” as used herein refers to the result of determining the average level of each transcript that associates with disease recurrence for an entire patient cohort. This is used to construct for each patient a transcriptome profile physical map where the X-axis represents the chromosomal locus for each RNA species and the Y-axis represents, for each RNA species, the difference between the average level in the entire patient cohort and the level within the particular patient tumor, standardized by dividing by half the population interquartile range (IQR) for the RNA species. These graphs reveal striking patient to patient differences, for example various sizes and numbers of spike-like features (segments) and global clouds of data points that vary considerably in their dispersion, features that can be tested as candidate biomarkers for risk of disease recurrence. A “TPM feature” refers to a measure of TPM dispersion that associates with a particular outcome, such as the rate of recurrence of disease, for example, recurrence of breast cancer. Measures of TPM dispersion may include mean absolute deviation from median and mean absolute deviation location to location.

### Methods of Generating Mapped Strings

Mapped strings may be generated by first determining the abundance of a gene expression species (RNA or protein) as measured by techniques including next generation sequencing RNA-Seq, RT-PCR, expression arrays, or proteomic methods such as Western blots and mass spectrometry.

The gene expression data may then be normalized according to the methods described herein, for example, by 3^{rd} quartile, candidate reference genes.

Next, the level of RNA or protein expression may be correlated with a clinical outcome, such as prognosis or prediction of cancer, diabetes, inflammatory diseases, neurodegenerative diseases, or heart disease. For each RNA or protein species, a degree of association to the clinical outcome, including magnitude and direction, may be estimated. A criterion for significant association (based on, for example, false discovery rate (FDR, q-value) or statistical significance (p-value,)) may then be established.

RNA or protein species that are significantly associated with the clinical outcome may be carried forward and assigned to their corresponding gene position on a human chromosomal coordinate physical map, for example, human genome browser hg19 assembly, UCSC.

From the chromosomally ordered representation of genes described above, mapped strings may be defined as an uninterrupted sequence of genes that have the same direction of association with outcome (e.g. good prognosis constituting one direction of association and bad prognosis representing the opposite direction of association). Mapped strings may be defined, however, in various ways, for example by minimum number of genes (or by introns found within a minimum number of genes) or by physical boundary. Mapped strings may also be restricted by other measures such as functional homogeneity or co-expression.

In some embodiments, mapped strings are defined by a minimum number of genes or introns found within a minimum number of genes. For example, mapped strings may be defined by at least 2, at least 3, at least 4, at least 5, at least 6, at least 7, at least 8, at least 9, at least 10, at least 15, at least 20, at least 25, at least 30, at least 35, at least 40, or at least 45 genes. In other embodiments, mapped strings are defined by physical boundary, for example, the end of a chromosome or the end of a chromosomal arm. In some embodiments, mapped strings are defined by functional homogeneity, for example groups of genes with similar function, such as proliferative genes or genes belonging to a common signaling pathway. In other embodiments, mapped strings are defined by genes that co-express with one another, for example, gene groups that have a co-expression value of R>0.4. In various embodiments, mapped strings are defined by any of the above aspects, alone or in combination.

Mapped strings can be represented as metagenes which can be used to create univariate or multivariate models that predict clinical outcomes. The quantitative contribution of individual genes in these metagenes can be set in a variety of ways. For example, the standardized normalized measure of the abundance of each RNA or protein species can be summed.

### Methods of Generating Transcriptome Profile Maps

Transcriptome profile maps (TPMs) may be generated by first measuring the amounts of individual gene expression product species in a sample of patient tissue. For example, the gene expression product species could be RNA or protein species. For example, the tissue could be blood, or solid tissue, and could be healthy or diseased (such as tumor) tissue. Several different technology platforms could be used to make these measurements, e.g., RNA-Seq, DNA microarrays, mass spectrometry, or ELISA.

Next, the measured amounts of the species can be normalized to compensate for variation in tissue amount or integrity from patient to patient. Examples of normalization methods include third quartile normalization, normalization using global expression, and reference gene normalization.

The central tendency of the normalized level of each RNA or protein species for the entire investigated patient population, for example, the median or the mean level, can then be calculated. This step could also be carried out for a patient population that is different from the study population but which has similar attributes (e.g., all estrogen receptor positive early breast cancer). In one embodiment, the level of each species is standardized to produce a centered, scaled score, for example, a robust z score such as z=(level−median)/(0.5*(3rd population quartile−1st population quartile)).

Additionally, a graphic (transcriptome physical map) can then be created for each patient study tissue in the study population, wherein the X-axis represents the chromosomal locus for each RNA or protein species gene and the Y-axis represents, for that species, the patient's standardized, normalized abundance value. In certain embodiments, gene expression product species present at low abundance or low population variance in abundance (e.g., below the 50th percentile) can be excluded from transcriptome physical maps.

Next, the data density features of these transcriptome physical maps can be mined for biomarkers of patient clinical outcomes, for example sensitivity or resistance to certain drugs, or progression from early cancer to a more advanced stage, or survival. Examples of such map density features are global dispersion of transcriptome z-scores, and number of Y-axis map segments. Map segments can be defined and quantified in several ways (e.g, by using circular binary segmentation or piecewise constant fitting programs). Individual segments are also evaluated and defined by minimum z-score cutoffs and minimum number of gene product species in a row on the same side of the Y-axis zero value.

Correlating Level of RNA Transcripts or their Expression Products to a Clinical Outcome

One skilled in the art will recognize that there are many statistical methods that may be used to determine whether there is a correlation between an outcome of interest (e.g., likelihood of survival) and levels of RNA or protein. Similarly, there are many statistical methods that may be used to determine whether there is a correlation between an outcome of interest (e.g., likelihood of survival) and TPM features as described here. This relationship can be presented as a continuous recurrence score (RS), or patients may be stratified into risk groups (e.g., low, intermediate, high). For example, a Cox proportional hazards regression model may fit to a particular clinical endpoint (e.g., RFI, DFS, OS). One assumption of the Cox proportional hazards regression model is the proportional hazards assumption, i.e. the assumption that effect parameters multiply the underlying hazard. Assessments of model adequacy may be performed including, but not limited to, examination of the cumulative sum of martingale residuals. One skilled in the art would recognize that there are numerous statistical methods that may be used to fit a flexible parametric model using the hazard scale and the Weibull distribution with natural spline smoothing of the log cumulative hazards function, with effects for treatment (chemotherapy or observation) and RS allowed to be time-dependent. See, e.g., P. Royston, M. Parmer, Statistics in Medicine 21:2175-2197, 2002).

Any of the methods described may group the levels of RNA transcripts or their expression products. The grouping of the RNA transcripts or expression products may be performed at least in part based on creation of mapped strings and/or TPMs as described herein. The formation of groups, in addition, can facilitate the mathematical weighting of the contribution of various expression levels to the recurrence/likelihood score. The weighting of a gene grouping representing a physiological process or component cellular characteristic can reflect the contribution of that process or characteristic to the pathology of the cancer and clinical outcome. Accordingly, the present invention provides gene groupings of the RNA transcripts, or their expression products, identified herein for use in the methods disclosed herein.

Methods to Predict Likelihood of Long-Term Survival without Breast Cancer Recurrence

As described above, a number of coding and non-coding RNA transcripts that correlate with breast cancer prognosis were identified previously. The levels of these RNA transcripts, or their expression products, can be determined in a tumor sample obtained from an individual patient who has breast cancer and for whom treatment is being contemplated. Depending on the outcome of the assessment, treatment with chemotherapy may be indicated, or an alternative treatment regimen may be indicated.

In carrying out the method of the present invention, a tumor sample is assayed or measured for a level of an RNA transcript, or its expression product. The tumor sample can be obtained from a solid tumor, e.g., via biopsy, or from a surgical procedure carried out to remove a tumor; or from a tissue or bodily fluid that contains cancer cells. In an embodiment of the invention, the tumor sample is obtained from a patient with breast cancer, such as ER-positive breast cancer. In another embodiment, the level of an RNA transcript, or its expression product, is normalized relative to the level of one or more reference RNA transcripts, or its expression product.

Methods of Assaying Levels of RNA Transcripts or their Expression Products

Methods of expression profiling include methods based on sequencing of polynucleotides, methods based on hybridization analysis of polynucleotides, and proteomics-based methods. Representative methods for sequencing-based analysis include Massively Parallel Sequencing (see e.g., Tucker et al., The American J. Human Genetics 85:142-154, 2009) and Serial Analysis of Gene Expression (SAGE). Exemplary methods known in the art for the quantification of mRNA expression in a sample include northern blotting and in situ hybridization (Parker & Barnes, Methods in Molecular Biology 106:247-283, 1999); RNAse protection assays (Hod, Biotechniques 13:852-854, 1992); and PCR-based methods, such as reverse transcription polymerase chain reaction (RT-PCR) (Weis et al., Trends in Genetics 8:263-264, 1992). Antibodies may be employed that can recognize sequence-specific duplexes, including DNA duplexes, RNA duplexes, and DNA-RNA hybrid duplexes or DNA-protein duplexes.

### Nucleic Acid Sequencing-Based Methods

Nucleic acid sequencing technologies are suitable methods for expression analysis. The principle underlying these methods is that the number of times a cDNA sequence is detected in a sample is directly related to the relative RNA levels corresponding to that sequence. These methods are sometimes referred to by the term Digital Gene Expression (DGE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000).

More recently, the advent of “next-generation” sequencing technologies has made DGE simpler, higher throughput, and more affordable. As a result, more laboratories are able to utilize DGE to screen the expression of more nucleic acids in more individual patient samples than previously possible. See, e.g., J. Marioni, Genome Research 18(9):1509-1517 (2008); R. Morin, Genome Research 18(4):610-621 (2008); A. Mortazavi, Nature Methods 5(7):621-628 (2008); N. Cloonan, Nature Methods 5(7):613-619 (2008). Massively parallel sequencing methods have also enabled whole genome or transcriptome sequencing, allowing the analysis of not only coding but also non-coding sequences. As reviewed in Tucker et al., The American J. Human Genetics 85:142-154 (2009), there are several commercially available massively parallel sequencing platforms, such as the Illumina Genome Analyzer (Illumina, Inc., San Diego, Calif.), Ion Torrent Sequencer (Life Technologies, Carlsbad, Calif.), Roche GS-FLX 454 Genome Sequencer (Roche Applied Science, Germany), and the Helicos® Genetic Analysis Platform (Helicos Biosciences Corp., Cambridge, Mass.). Other developing technologies may be used.

### Reverse Transcription PCR (RT-PCR)

The starting material is typically total RNA isolated from a human tumor, usually from a primary tumor. Optionally, normal tissues from the same patient can be used as an internal control. RNA can be extracted from a tissue sample, e.g., from a sample that is fresh, frozen (e.g. fresh frozen), or paraffin-embedded and fixed (e.g. formalin-fixed).

General methods for RNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from a tumor sample can be isolated, for example, by cesium chloride density gradient centrifugation. The isolated RNA may then be depleted of ribosomal RNA as described in U.S. Pub. No. 2011/0111409.

The sample containing the RNA is then subjected to reverse transcription to produce cDNA from the RNA template, followed by exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

PCR-based methods use a thermostable DNA-dependent DNA polymerase, such as a Taq DNA polymerase. For example, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction product. A third oligonucleotide, or probe, can be designed to facilitate detection of a nucleotide sequence of the amplicon located between the hybridization sites of the two PCR primers. The probe can be detectably labeled, e.g., with a reporter dye, and can further be provided with both a fluorescent dye, and a quencher fluorescent dye, as in a Taqman® probe configuration. Where a Taqman® probe is used, during the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 384-well format on a thermocycler. The RT-PCR may be performed in triplicate wells with an equivalent of 2 ng RNA input per 10 μL-reaction volume. During amplification, laser-induced fluorescent signal is collected in real-time through fiber optics cables for all wells, and detected at the CCD. The system includes software for running the instrument and for analyzing the data.

5′-Nuclease assay data are generally initially expressed as a threshold cycle (“Cₜ”). Fluorescence values are recorded during every cycle and represent the amount of product amplified to that point in the amplification reaction. The threshold cycle (Cₜ) is generally described as the point when the fluorescent signal is first recorded as statistically significant.

To minimize errors and the effect of sample-to-sample variation, RT-PCR is usually performed using an internal standard. The ideal internal standard gene (also referred to as a reference gene) is expressed at a constant level among cancerous and non-cancerous tissue of the same origin (i.e., a level that is not significantly different among normal and cancerous tissues), and is not significantly affected by the experimental treatment (i.e., does not exhibit a significant difference in expression level in the relevant tissue as a result of exposure to chemotherapy). RNAs most frequently used to normalize patterns of gene expression are mRNAs for the housekeeping genes glyceraldehyde-3-phosphate-dehydrogenase (GAPDH) and β-actin. Gene expression measurements can be normalized relative to the mean of one or more (e.g., 2, 3, 4, 5, or more) reference genes. Reference-normalized expression measurements can range from 0 to 15, where a one unit increase generally reflects a 2-fold increase in RNA quantity.

Real time PCR is compatible both with quantitative competitive PCR, where an internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

### Design of PCR Primers and Probes

PCR primers and probes can be designed based upon exon, intron, or intergenic sequences present in the RNA transcript of interest. Primer/probe design can be performed using publicly available software, such as the DNA BLAT software developed by Kent, W. J., Genome Res. 12(4):656-64 (2002), or by the BLAST software including its variations.

Where necessary or desired, repetitive sequences of the target sequence can be masked to mitigate non-specific signals. Exemplary tools to accomplish this include the Repeat Masker program available on-line through the Baylor College of Medicine, which screens DNA sequences against a library of repetitive elements and returns a query sequence in which the repetitive elements are masked. The masked sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Rrawetz S, Misener S (eds) Bioinformatics Methods and Protocols: Methods in Molecular Biology. Humana Press, Totowa, N.J., pp 365-386).

Other factors that can influence PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases, and exhibit Tm's between 50 and 80° C., e.g. about 50 to 70° C.

For further guidelines for PCR primer and probe design see, e.g. Dieffenbach, C W. et al, “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods MoI. Biol. 70:520-527 (1997), the entire disclosures of which are hereby expressly incorporated by reference.

### MassARRAY® System

In MassARRAY-based methods, such as the exemplary method developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derived PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see e.g., Ding and Cantor, Proc. Natl. Acad. Sci. USA 100:3059-3064 (2003).

### Other PCR-Based Methods

Further PCR-based techniques that can find use in the methods disclosed herein include, for example, BeadArray® technology (Illumina, San Diego, Calif.; Oliphant et al., Discovery of Markers for Disease (Supplement to Biotechniques), June 2002; Ferguson et al., Analytical Chemistry 72:5618, 2000); BeadsArray for Detection of Gene Expression® (BADGE), using the commercially available Luminex1OO LabMAP® system and multiple color-coded microspheres (Luminex Corp., Austin, Tex.) in a rapid assay for gene expression (Yang et al., Genome Res. 11:1888-1898, 2001); and high coverage expression profiling (HiCEP) analysis (Fukumura et al., Nucl. Acids. Res. 31(16) e94, 2003).

### Microarrays

In this method, polynucleotide sequences of interest (including cDNAs and oligonucleotides) are arrayed on a substrate. The arrayed sequences are then contacted under conditions suitable for specific hybridization with detectably labeled cDNA generated from RNA of a sample. The source of RNA typically is total RNA isolated from a tumor sample, and optionally from normal tissue of the same patient as an internal control or cell lines. RNA can be extracted, for example, from frozen or archived paraffin-embedded and fixed (e.g. formalin-fixed) tissue samples.

For example, PCR amplified inserts of cDNA clones of a gene to be assayed are applied to a substrate in a dense array. Usually at least 10,000 nucleotide sequences are applied to the substrate. For example, the microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After washing under stringent conditions to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA are hybridized pair wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et at, Proc. Natl. Acad. Sci. USA 93(2):106-149, 1996). Microarray analysis can be performed on commercially available equipment, following the manufacturer's protocols, such as by using the Affymetrix GenChip® technology, or Incyte's microarray technology.

Isolating RNA from Body Fluids

Methods of isolating RNA for expression analysis from blood, plasma and serum (see for example, Tsui N B et al. Clin. Chem. 48, 1647-53, 2002 and references cited therein) and from urine (see for example, Boom R et al. J Clin Microbiol. 28, 495-503, 1990 and references cited therein) have been described.

### Immunohistochemistry

Immunohistochemistry methods are also suitable for detecting the expression levels of genes and applied to the method disclosed herein. Antibodies (e.g., monoclonal antibodies) that specifically bind a gene product of a gene of interest can be used in such methods. The antibodies can be detected by direct labeling of the antibodies themselves, for example, with radioactive labels, fluorescent labels, hapten labels such as biotin, or an enzyme such as horse radish peroxidase or alkaline phosphatase. Alternatively, unlabeled primary antibody can be used in conjunction with a labeled secondary antibody specific for the primary antibody. Immunohistochemistry protocols and kits are well known in the art and are commercially available.

### Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics.

General Description of the RNA Isolation and Preparation from Fixed, Paraffin-Embedded Samples for Whole Transcriptome Sequencing

The steps of a representative protocol for profiling gene expression levels using fixed, paraffin-embedded tissues as the RNA source are provided in various published journal articles. (See, e.g., T. E. Godfrey et al., J. Molec. Diagnostics 2: 84-91, 2000; K. Specht et al., Am. J. Pathol. 158: 419-29, 2001, M. Cronin, et al., Am J Pathol 164:35-42, 2004). Modified methods can used for whole transcriptome sequencing as described in the Examples section. Briefly, a representative process starts with cutting a tissue sample section (e.g. about 10 μm thick sections of a paraffin-embedded tumor tissue sample). The RNA is then extracted, and ribosomal RNA may be deleted as described in U.S. Pub. No. 2011/0111409. cDNA sequencing libraries may be prepared that are directional and single or paired-end using commercially available kits such as the ScriptSeq™ mRNA-Seq Library Preparation Kit (Epicenter Biotechnologies, Madison, Wis.). The libraries may also be barcoded for multiplex sequencing using commercially available barcode primers such as the RNA-Seq Barcode Primers from Epicenter Biotechnologies (Madison, Wis.). PCR is then carried out to generate the second strand of cDNA to incorporate the barcodes and to amplify the libraries. After the libraries are quantified, the sequencing libraries may be sequenced as described herein.

### Coexpression Analysis

To perform particular biological processes, genes often work together in a concerted way, i.e. they are co-expressed. Co-expressed gene networks identified for a disease process like cancer can also serve as prognostic biomarkers. Such co-expressed genes can be assayed in lieu of, or in addition to, assaying the biomarker with which they co-express.

One skilled in the art will recognize that many co-expression analysis methods now known or later developed will fall within the scope and spirit of the present invention. These methods may incorporate, for example, correlation coefficients, co-expression network analysis, clique analysis, etc., and may be based on expression data from RT-PCR, microarrays, sequencing, and other similar technologies. For example, gene expression clusters can be identified using pair-wise analysis of correlation based on Pearson or Spearman correlation coefficients. (See e.g, Pearson K. and Lee A., Biometrika 2:357, 1902; C. Spearman, Amer. J. Psychol. 15:72-101, 1904; J. Myers, A. Well, Research Design and Statistical Analysis, p. 508, 2^{nd} Ed., 2003) In general, a correlation coefficient of equal to or greater than 0.3 is considered to be statistically significant in a sample size of at least 20. (See e.g., G. Norman, D. Streiner, Biostatistics: The Bare Essentials, 137-138, 3^{rd} Ed. 2007)

### Reference Normalization

In order to minimize expression measurement variations due to non-biological variations in samples, e.g., the amount and quality of product to be measured, the level of an RNA transcript or its expression product may be normalized relative to the mean levels obtained for one or more reference RNA transcripts or their expression products. Examples of reference RNA transcripts or expression products include housekeeping genes, such as GAPDH. Alternatively, all of the assayed RNA transcripts or expression products, or a subset thereof, may also serve as reference. On a transcript (or protein)-by-transcript (or protein) basis, measured normalized amount of a patient tumor RNA or protein may be compared to the amount found in a cancer tissue reference set. See e.g., Cronin, M. et al., Am. Soc. Investigative Pathology 164:35-42 (2004). The normalization may be carried out such that a one unit increase in normalized level of an RNA transcript or expression product generally reflects a 2-fold increase in quantity present in the sample.

### Kits of the Invention

The materials for use in the methods of the present invention are suited for preparation of kits produced in accordance with well-known procedures. The present invention thus provides kits comprising agents, which may include primers and/or probes, for quantitating the level of the disclosed RNA transcripts or their expression products via methods such as whole transcriptome sequencing or RT-PCR for predicting prognostic outcome. Such kits may optionally contain reagents for the extraction of RNA from tumor samples, in particular, fixed paraffin-embedded tissue samples and/or reagents for whole transcriptome sequencing. In addition, the kits may optionally comprise the reagent(s) with an identifying description or label or instructions relating to their use in the methods of the present invention. The kits may comprise containers (including microliter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase). Mathematical algorithms used to estimate or quantify prognostic information are also potential components of kits.

### Reports

The methods of this invention are suited for the preparation of reports summarizing the predictions resulting from the methods of the present invention. A “report” as described herein, is an electronic or tangible document that includes elements that provide information of interest relating to a likelihood assessment and its results. A subject report includes at least a likelihood assessment, e.g., an indication as to the likelihood that a cancer patient will exhibit long-term survival without breast cancer recurrence. A subject report can be completely or partially electronically generated, e.g., presented on an electronic display (e.g., computer monitor). A report can further include one or more of: 1) information regarding the testing facility; 2) service provider information; 3) patient data; 4) sample data; 5) an interpretive report, which can include various information including: a) indication; b) test data, where test data can include a normalized level of one or more RNA transcripts of interest, and 6) other features.

The present invention therefore provides methods of creating reports and the reports resulting therefrom. The report may include a summary of the levels of the RNA transcripts, or the expression products of such RNA transcripts, in the cells obtained from the patient's tumor sample. The report may include a prediction that the patient has an increased likelihood of long-term survival without breast cancer recurrence or the report may include a prediction that the subject has a decreased likelihood of long-term survival without breast cancer recurrence. The report may include a recommendation for a treatment modality such as surgery alone or surgery in combination with chemotherapy. The report may be presented in electronic format or on paper.

Thus, in some embodiments, the methods of the present invention further include generating a report that includes information regarding the patient's likelihood of long-term survival without breast cancer recurrence. For example, the methods of the present invention can further include a step of generating or outputting a report providing the results of a patient response likelihood assessment, which can be provided in the form of an electronic medium (e.g., an electronic display on a computer monitor), or in the form of a tangible medium (e.g., a report printed on paper or other tangible medium).

A report that includes information regarding the likelihood that a patient will exhibit long-term survival without breast cancer recurrence, is provided to a user. An assessment as to the likelihood that a cancer patient will exhibit long-term survival without breast cancer recurrence, is referred to as a “likelihood assessment.” A person or entity who prepares a report (“report generator”) may also perform the likelihood assessment. The report generator may also perform one or more of sample gathering, sample processing, and data generation, e.g., the report generator may also perform one or more of: a) sample gathering; b) sample processing; c) measuring a level of an RNA transcript or its expression product; d) measuring a level of a reference RNA transcript or its expression product; and e) determining a normalized level of an RNA transcript or its expression product. Alternatively, an entity other than the report generator can perform one or more sample gathering, sample processing, and data generation.

The term “user” or “client” refers to a person or entity to whom a report is transmitted, and may be the same person or entity who does one or more of the following: a) collects a sample; b) processes a sample; c) provides a sample or a processed sample; and d) generates data for use in the likelihood assessment. In some cases, the person or entity who provides sample collection and/or sample processing and/or data generation, and the person who receives the results and/or report may be different persons, but are both referred to as “users” or “clients.” In certain embodiments, e.g., where the methods are completely executed on a single computer, the user or client provides for data input and review of data output. A “user” can be a health professional (e.g., a clinician, a laboratory technician, a physician (e.g., an oncologist, surgeon, pathologist), etc.).

In embodiments where the user only executes a portion of the method, the individual who, after computerized data processing according to the methods of the invention, reviews data output (e.g., results prior to release to provide a complete report, a complete, or reviews an “incomplete” report and provides for manual intervention and completion of an interpretive report) is referred to herein as a “reviewer.” The reviewer may be located at a location remote to the user (e.g., at a service provided separate from a healthcare facility where a user may be located).

Where government regulations or other restrictions apply (e.g., requirements by health, malpractice, or liability insurance), all results, whether generated wholly or partially electronically, are subjected to a quality control routine prior to release to the user.

### Computer-Based Systems and Methods

The methods and systems described herein can be implemented in numerous ways. In one embodiment of the invention, the methods involve use of a communications infrastructure, for example, the internet. Several embodiments of the invention are discussed below. The present invention may also be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

In an embodiment of the invention, during or after data input by the user, portions of the data processing can be performed in the user-side computing environment. For example, the user-side computing environment can be programmed to provide for defined test codes to denote a likelihood “score,” where the score is transmitted as processed or partially processed responses to the reviewer's computing environment in the form of test code for subsequent execution of one or more algorithms to provide a result and/or generate a report in the reviewer's computing environment. The score can be a numerical score (representative of a numerical value) or a non-numerical score representative of a numerical value or range of numerical values (e.g., “A”: representative of a 90-95% likelihood of a positive response; “High”: representative of a greater than 50% chance of a positive response (or some other selected threshold of likelihood); “Low”: representative of a less than 50% chance of a positive response (or some other selected threshold of likelihood), and the like.

As a computer system, the system generally includes a processor unit. The processor unit operates to receive information, which can include test data (e.g., level of an RNA transcript or its expression product; level of a reference RNA transcript or its expression product; normalized level of an RNA transcript or its expression product) and may also include other data such as patient data. This information received can be stored at least temporarily in a database, and data analyzed to generate a report as described above.

Part or all of the input and output data can also be sent electronically. Certain output data (e.g., reports) can be sent electronically or telephonically (e.g., by facsimile, using devices such as fax back). Exemplary output receiving devices can include a display element, a printer, a facsimile device and the like. Electronic forms of transmission and/or display can include email, interactive television, and the like. In an embodiment of the invention, all or a portion of the input data and/or output data (e.g., usually at least the final report) are maintained on a web server for access, preferably confidential access, with typical browsers. The data may be accessed or sent to health professionals as desired. The input and output data, including all or a portion of the final report, can be used to populate a patient's medical record that may exist in a confidential database as the healthcare facility.

The present invention also contemplates a computer-readable storage medium (e.g., CD-ROM, memory key, flash memory card, diskette, etc.) having stored thereon a program which, when executed in a computing environment, provides for implementation of algorithms to carry out all or a portion of the results of a likelihood assessment as described herein. Where the computer-readable medium contains a complete program for carrying out the methods described herein, the program includes program instructions for collecting, analyzing and generating output, and generally includes computer readable code devices for interacting with a user as described herein, processing that data in conjunction with analytical information, and generating unique printed or electronic media for that user.

Where the storage medium includes a program that provides for implementation of a portion of the methods described herein (e.g., the user-side aspect of the methods, (e.g., data input, report receipt capabilities, etc.), the program provides for transmission of data input by the user (e.g., via the internet, via an intranet, etc.) to a computing environment at a remote site. Processing or completion of processing of the data is carried out at the remote site to generate a report. After review of the report, and completion of any needed manual intervention, to provide a complete report, the complete report is then transmitted back to the user as an electronic document or printed document (e.g., fax or mailed paper report). The storage medium containing a program according to the invention can be packaged with instructions (e.g., for program installation, use, etc.) recorded on a suitable substrate or a web address where such instructions may be obtained. The computer-readable storage medium can also be provided in combination with one or more reagents for carrying out a likelihood assessment (e.g., primers, probes, arrays, or such other kit components).

Having described the invention, the same will be more readily understood through reference to the following Examples, which are provided by way of illustration, and are not intended to limit the invention in any way. All citations through the disclosure are hereby expressly incorporated by reference.

### EXAMPLES

### Example 1

### Materials and Methods

Unless otherwise noted, the materials and methods described in Examples 1 and 2 are also described in PCT/US2012/063313, filed Nov. 2, 2012, now WO 2013/070521, which is incorporated by reference in its entirety. Furthermore, accession numbers of genes and intronic coordinates for genes and introns listed in the present disclosure are identified in PCT/US2012/063313, filed Nov. 2, 2012, now WO 2013/070521.

### Patients

One hundred and thirty-six primary breast cancer FFPE tumor specimens with clinical outcomes were provided by Providence St. Joseph Medical Center (Burbank, Calif.), with institutional review board approval. The time to first recurrence of breast cancer or death due to breast cancer (including death due to unknown cause) was determined from these records. Patients who were still alive without breast cancer recurrence or who died due to known other causes were considered censored at the time of last follow-up or death. These tumor specimens were used for biomarker discovery in the development of the Oncotype DX® assay. See e.g., U.S. Pat. No. 7,081,340; S. Paik et al., The New England Journal of Medicine 351, 2817, 2004). For the present study, 136 specimens had adequate RNA remaining. Among the 136 patients, 26 experienced breast cancer recurrence or death due to breast cancer.

### RNA-Seq Sample Preparation and Sequencing

Total RNA was prepared from three 10-μm-thick sections of FFPE tumor tissue as previously described using the MasterPure™ Purification Kit (Epicentre® Biotechnologies, Madison, Wis.). M. Cronin et al.; Am. J. Pathol. 164, 35-42, 2004. One hundred nanograms of the isolated RNA were depleted of ribosomal RNA as described. See U.S. Pub. No. 2011/0111409. Sequencing libraries for whole transcriptome analysis were prepared using ScriptSeq™ mRNA-Seq Library Preparation Kits (Epicentre® Biotechnologies, Madison, Wis.). During the cDNA synthesis step, additional incubation for 90 minutes at 37° C. was implemented in the reverse transcription step to increase library yield. After 3′-terminal tagging, the di-tagged cDNA was purified using MinElute® PCR Purification Kits (Qiagen, Valencia, Calif.). Two 6 base index sequences were used to prepare barcoded libraries for duplex sequencing (RNA-Seq Barcode Primers; Epicentre® Biotechnologies, Madison, Wis.). PCR was carried out through 16 cycles to generate the second strand of cDNA, incorporate barcodes, and amplify libraries. The amplified libraries were size-selected by a solid phase reversible immobilization, paramagnetic bead-based process (Agencourt® AMPure® XP System; Beckman Coulter Genomics, Danvers, Mass.). Libraries were quantified by PicoGreen® assay (Life Technologies, Carlsbad, Calif.) and visualized with an Agilent Bioanalyzer using a DNA 1000 kit (Agilent Technologies, Waldbronn, Germany).

TruSeq™ SR Cluster Kits v2 (Illumina Inc.; San Diego, Calif.) were used for cluster generation in an Illumina cBOT™ instrument following the manufacturer's protocol. Two indexed libraries were loaded into each lane of flow cells. Sequencing was performed on an Illumina HiSeq®2000 instrument (Illumina, Inc.) by the manufacturer's protocol. Multiplexed single-read runs were carried out with a total of 57 cycles per run (including 7 cycles for the index sequences).

### Data Quality Assessment

Each sequencing lane was duplexed with two patient sample libraries using a 6 base barcode to differentiate between them. The mean read ratio+/−SD between the two samples in each lane was 1.05±0.38 and the mean+/−SD percentage of un-discerned barcodes was 2.08%±1.63%. Using principal components analysis and other exploratory data analysis methods, no systematic differences were found among samples associated with flow cell or barcode.

In a run-in phase of the study, duplicate libraries were prepared for 8 samples selected at random from the study set of 136. RefSeq RNA coverage for these libraries ranged between 3.1M and 6.7M uniquely mapped reads. Log count Pearson correlations among duplicate libraries ranged between 0.947 and 0.985. Single libraries were prepared for the remaining 128 samples and distributed in duplex mode among the lanes of 8 flow-cells. Sequencing in 3 lanes failed. Two libraries had low yield, resulting in low coverage. Three lanes were flagged by various Illumina process monitoring indices: low Q30 (coverage=2.8M and 4.2M), high cluster density (coverage=1.6M and 1.8M), or inadequate imaging (coverage=3.3M and 3.1M). For the remaining lanes, sample coverage ranged between 2.5M and 7.3M reads. New libraries for the samples that had low yield were prepared and sequenced. Libraries in the failed and flagged lanes, as well as some of the low coverage samples, were re-sequenced. Replicate correlations among all sequenced samples were very high, 0.985 for the samples with the high cluster density in the original run, and over 0.990 for all others. For the analysis data set, data for one of each of the duplicate libraries from the run-in experiment were kept. For the samples for which new libraries were prepared and for the samples in the failed and flagged lanes, the reads from the subsequent run were used. For the samples with low coverage for which the library was reprocessed, reads from the two runs were pooled. For the rest of the samples, the reads from the single lane were used. Results differed little when other data analysis procedures were used, for example, using only the second run when libraries were reprocessed.

### Statistics and Bioinformatics

With the exception noted below, all primary analysis of sequence data was performed in CASAVA 1.7, the standard data processing package from Illumina. De-multiplexing of sample indices was set with 1 mismatch tolerance to separate the two samples within each lane. Raw FASTQ sequences were trimmed from both ends before mapping to the human genome (UCSC release, version 19), to address 3′ end adapter contamination and random RT primer artifacts, and 5′ end terminal-tagging oligonucleotide artifacts. The libraries as prepared contain strand-of-origin (directional) sequence information. Annotated RNA counts (defined by refFlat.txt from UCSC) were calculated by CASAVA 1.7 both with and without consideration of strand-of-origin information. Although retained in the mapping process, CASAVA does not provide directional counts by default. These counts were obtained by splitting the mapped (export.txt) file into two parts, one with sense strand counts, the other with antisense strand counts, and processing them independently. Raw FASTQ sequence was mapped with Bowtie (B. Langmead et al., Genome Biology 10, R25, (2009) in parallel with CASAVA to count ribosomal RNA transcripts.

Data were analyzed in 3 categories: first, RefSeq RNAs, about 80% of which are exon sequences, consolidated for each gene; second, intronic RNA sequences, consolidated for each gene; third, intergenic sequences. RNAs with maximum counts less than 5 among the 136 patients were excluded from analysis. Of 21,283 total RefSeq transcripts counted by CASAVA, 821 had a maximum count less than 5, leaving 20,462 RefSeq transcripts for analysis. Similar to a recently published procedure described by Bullard et al. (BMC Bioinformatics 11, 94, 2010), log 2 raw RNA counts (setting the log 2 for a 0 count to 0) were normalized by subtracting the 3rd quartile of the log 2 RefSeq RNA counts and adding the cohort mean 3rd quartile (“Q3 normalization”). For analysis of RefSeq and intergenic RNAs normalization, RefSeq RNA data were used. For analysis of intronic RNAs normalization, intronic RNA data were used.

Standardized hazard ratios for breast cancer recurrence for each RNA, that is, the proportional change in the hazard with a 1-standard deviation increase in the normalized level of the RNA, were calculated using univariate Cox proportional hazard regression analyses (Cox, Journal of the Royal Statistical Society: Series B (Methodological) 34, 187, 1972). The robust standard error estimate of Lin and Wei (Journal of the American Statistical Society, 84, 1074, 1989) was used to accommodate possible departures from the assumptions of Cox regression, including nonlinearity of the relationship of gene expression with log hazard and nonproportional hazards. False discovery rates (FDR, q-values) were assessed using the method of Storey (Journal of the Royal Statistical Society, Series B 64, 479, 2002) with a “tuning parameter” of λ=0.5. Analyses were conducted to identify true discovery degree of association (TDRDA) sets of RNAs with absolute standardized hazard ratio greater than a specified lower bound while controlling the FDR at 10% (Crager, Statistics in Medicine 29, 33-45, 2010). Taking individual RNAs identified at this FDR, the analysis finds the maximum lower bound for which the RNA is included in a TDRDA set. Also computed was an estimate of each RNA's actual standardized hazard ratio corrected for regression to the mean.

Expression of 192 transcripts in the same tumor RNAs was measured using previously described RT-PCR methods (Cronin et al., Am. J. Pathol. 164, 35-42, 2004; Cronin et al., Clinical Chemistry 50, 1464, 2004). Standardized hazard ratios associating the expression of each gene (normalized by subtracting each gene's crossing threshold (CT) from the cohort median CT) with cancer recurrence were computed using the same methods used for evaluation of the RNA-Seq data.

### Identifying Intergenic Sequences

Intergenic regions were identified by a novel program that evaluates genomic regions that vary widely in length and on a population basis. This program was developed to evaluate intergenic regions having wide variations in length, and to use data from a population of subjects rather than an individual subject. The uniquely mapped reads from all 136 patients were analyzed to identify clusters of reads that might arise from intergenic transcripts. Genomic regions containing less than 2 mapped reads of genomic sequence were not counted to eliminate potential noise from mis-mapping or genomic DNA contamination. The remaining reads were clustered into individual read “islands” based on the overlap of their mapped coordinates to the hg 19 reference human genome, which resulted in 12,750,071 islands in all 136 patient samples. Any islands within 30 base pairs (bp) of each other were grouped together as regions of interest (ROI) producing a total of 6,633,258 ROIs. The number of ROIs were further reduced by the following criteria: 1) The average number of reads mapped to the ROI was ≧5 across all 136 patients, 2) the length of the ROI was at least 100 bp, and 3) the read depth (average read number divided by the length of the ROI) was ≧0.075. Applying these criteria reduced the number of ROIs to 23,024. ROIs were classified as intergenic regions if they did not overlap with the transcripts (including non-coding ones) annotated in the refFlat.txt file obtained from UCSC, thereby eliminating overlap with known exons and introns of protein-coding genes and well annotated non-protein coding transcripts. A total of 2,101 intergenic regions were identified by this computational procedure.

### Example 2

### Evaluation of Whole Transcriptome RNA-Seq as a Platform for Biomarker Discovery

Patient clinical characteristics are shown in Table 1. One-hundred and ten patients (81%) had no involved nodes. There was a mixture of chemotherapy and hormonal therapy usage. Estrogen receptor (ER) status was not included in patient records. Therefore, normalized ESR1 mRNA levels obtained in the present RNA-Seq study were used to identify 111 tumors as estrogen-receptor positive and 25 as estrogen-receptor negative. Use of RT-PCR rather than RNA-Seq for this purpose yielded similar but not identical results, identifying as ER+ two more patients, for a total of 113. Archive ages of FFPE tumor blocks ranged from 5 to 12.4 years (median 8.5 years).

RNA-Seq results were successfully generated for all 136 patients, with an average of 43 million median reads per patient (86 million median reads per Illumina Hiseq 2000 flow cell lane). Sixty-nine percent of these uniquely mapped to the human genome: 19.2% to exons, 64.9% to introns, and 15.9% to intergenic regions. Ribosomal RNA accounted for less than 0.3% of the total reads. On average, 17,248 RefSeq transcripts were detected per patient, 66% with greater than 10 counts, and 47% with greater than 100 counts.

In the present set of experiments, gene expression was normalized using the 3^{rd} quartile (J. Bullard et al., BMC Bioinformatics, 11:94, 2010, expressed on the log₂ scale, and standardized to have standard deviation 1 across the study population. When combining genes into meta-gene strings with common direction of association, the standardized gene expressions were summed over the genes in the string to compute the meta-gene expression. Standardized log hazard ratios (that is, the change in the log hazard with a 1-standard deviation change in the covariate) were corrected for regression to the mean (M. Crager, Stat. Med. 29(1): 33-45, 2010) using the distribution of standardized log hazard ratios for all the individual genes in the study. This is expected to be a conservative adjustment (that is, over-correcting for regression to the mean), because in general the prognostic value of multiple genes models is great than the prognostic power of single gene models.

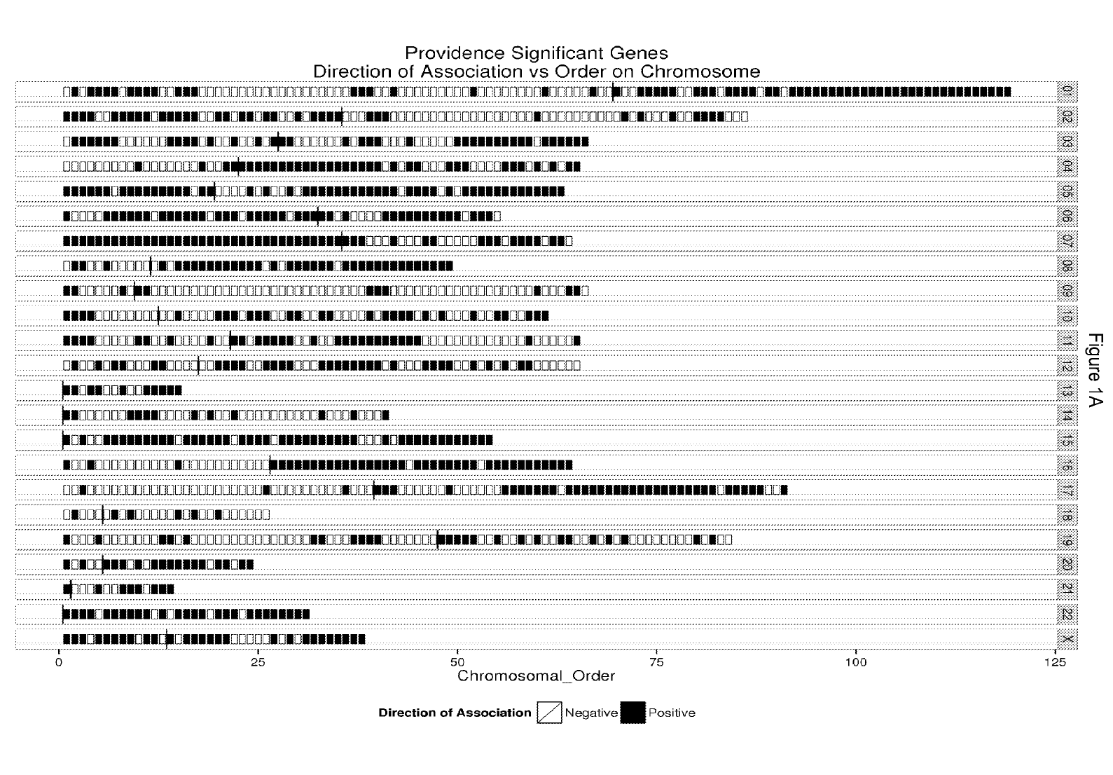

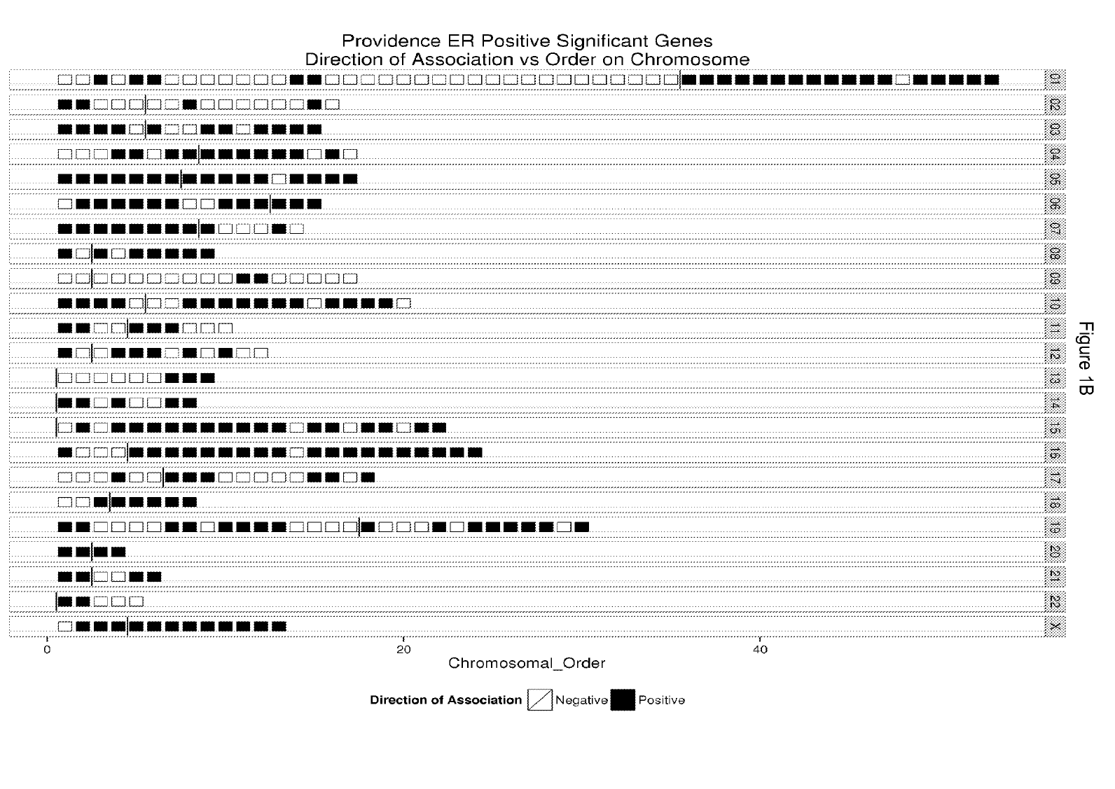

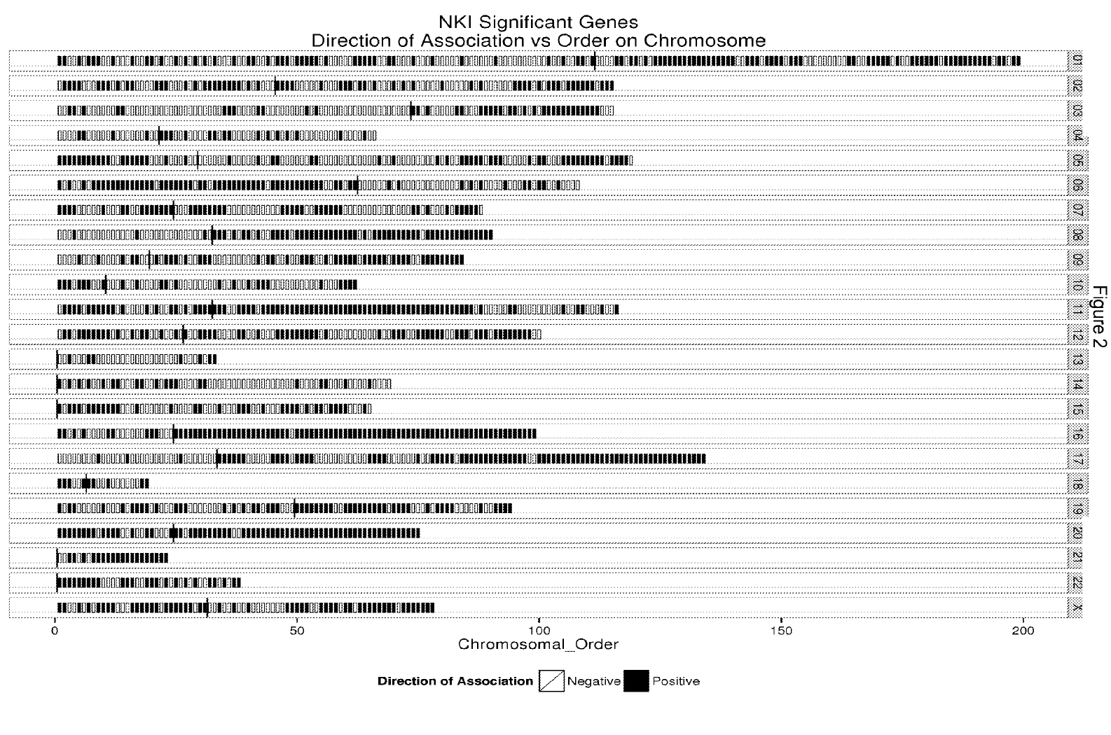

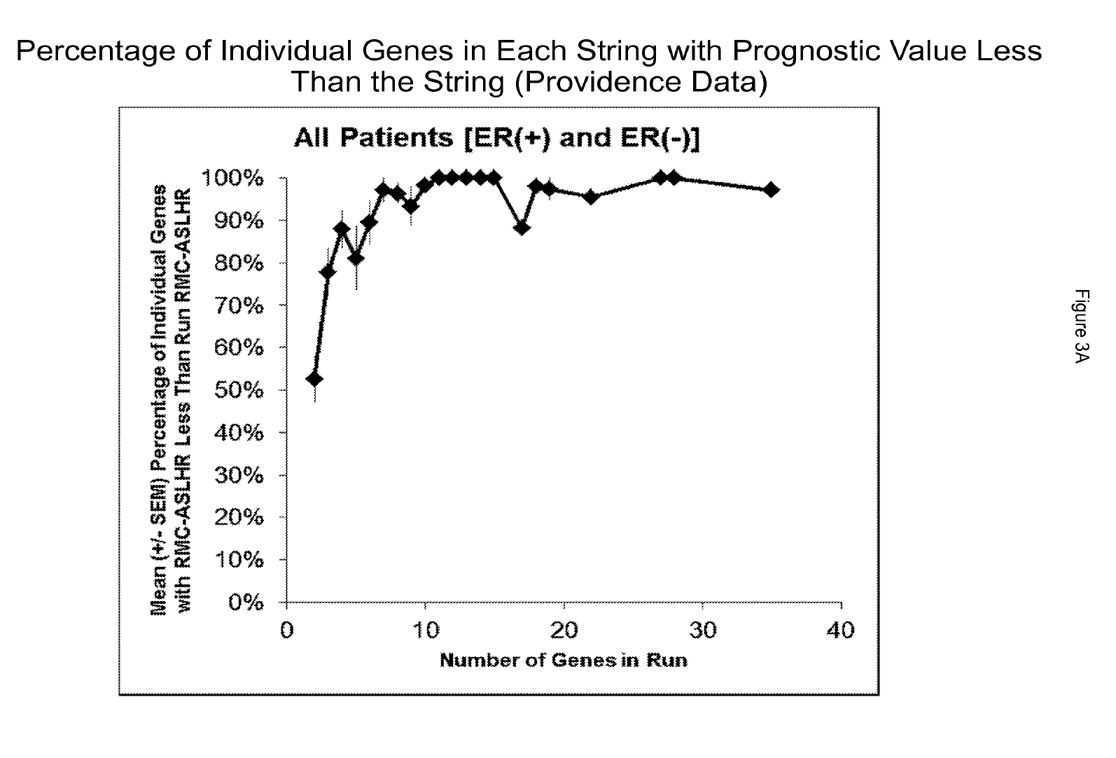

FIG. 1A of PCT/US2012/063313, filed Nov. 2, 2012, now WO 2013/070521 displays results from the historical RT-PCR 192 candidate gene screen of the Providence 136 patient cohort, relating increasing mRNA expression to recurrence risk hazard ratios and statistical significance. As shown, fourteen of the sixteen cancer-related genes in the Oncotype DX® panel were assayed, and most were identified with Hazard Ratios greater than 1.2 or less than 0.8 and P values <0.05.

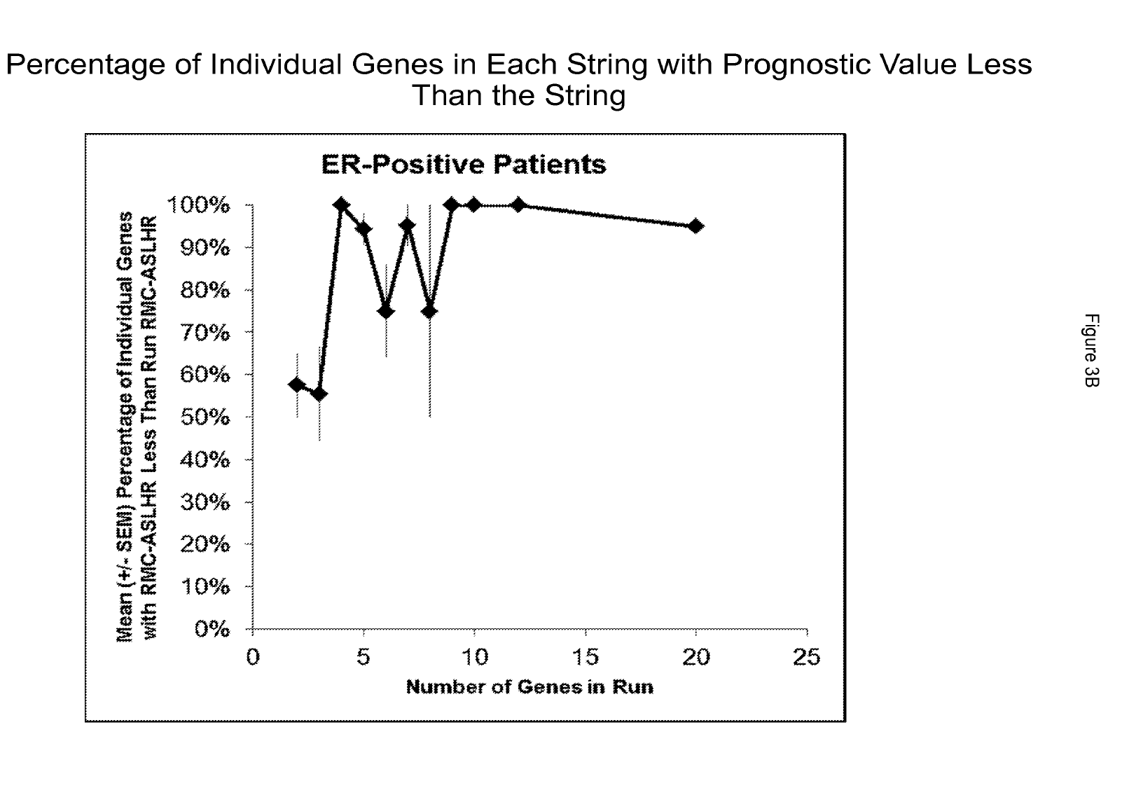

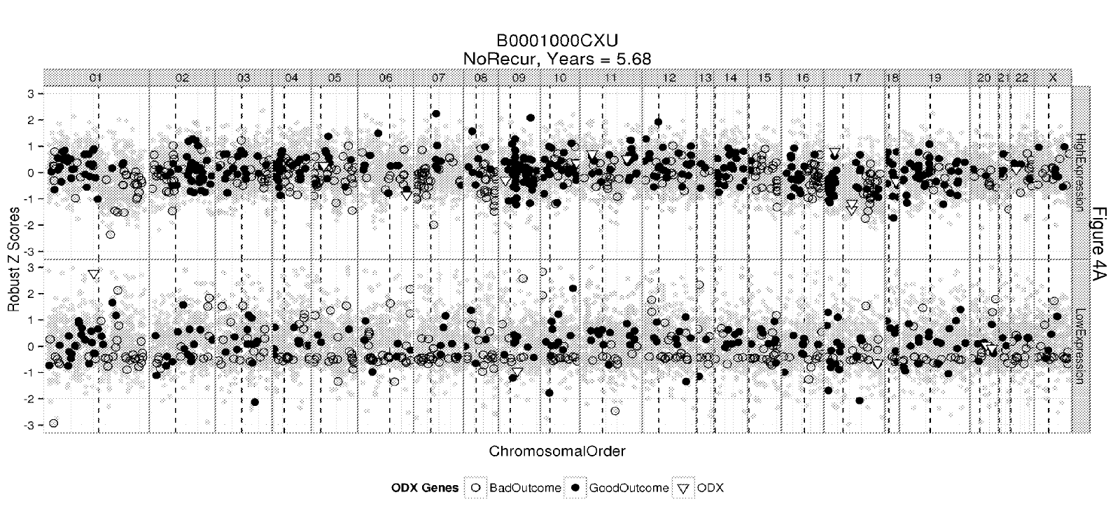

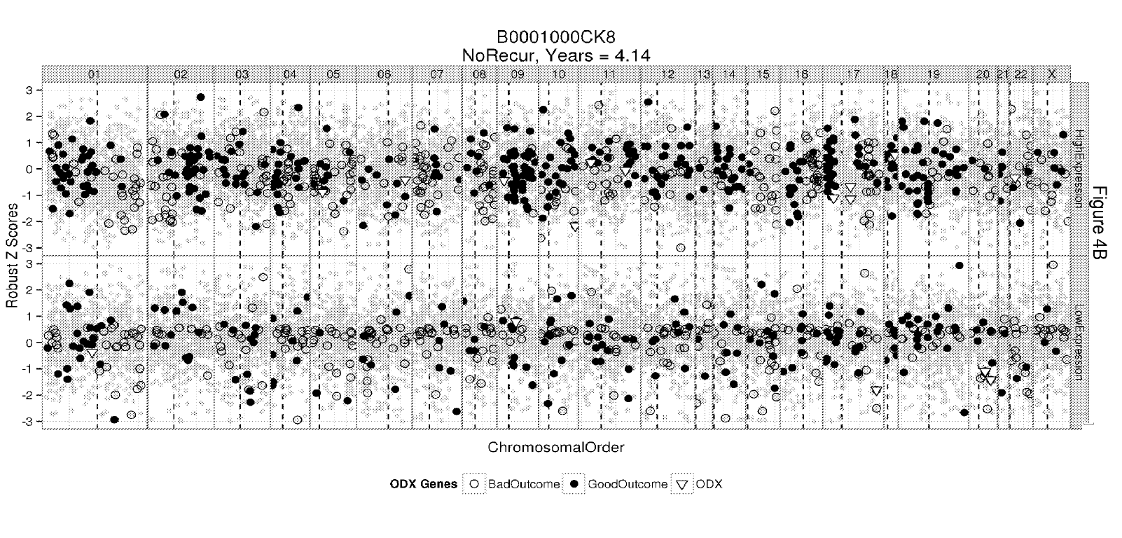

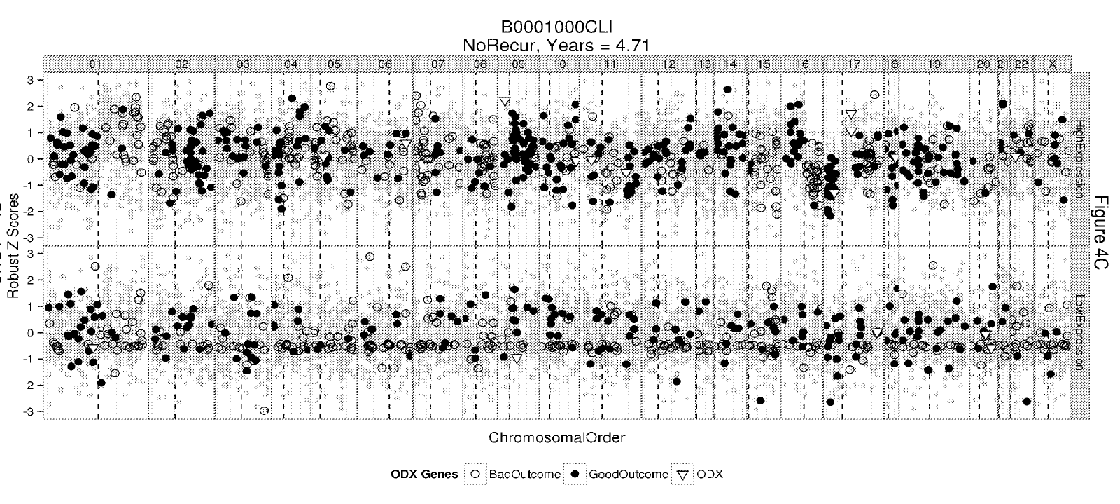

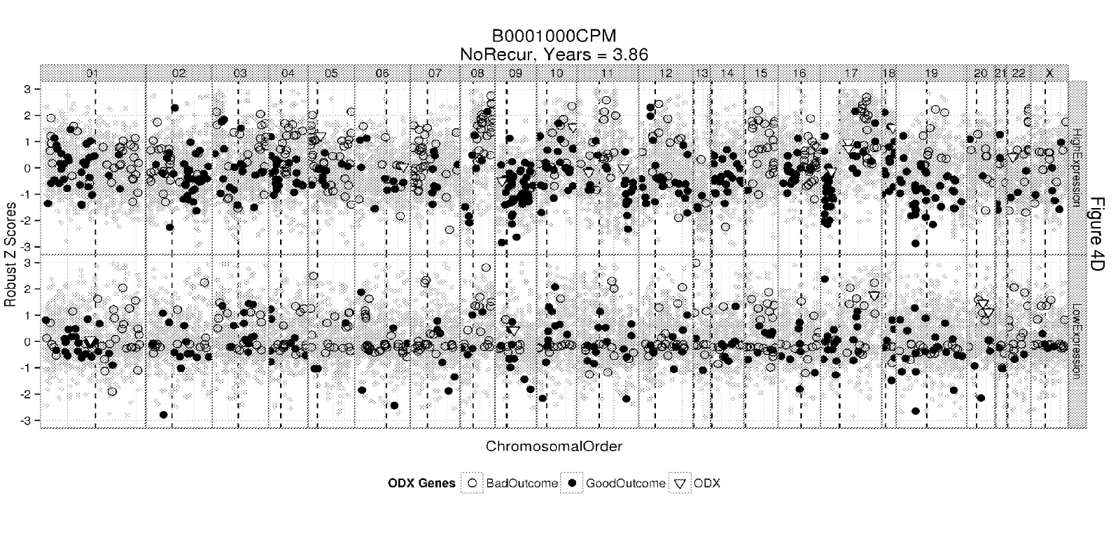

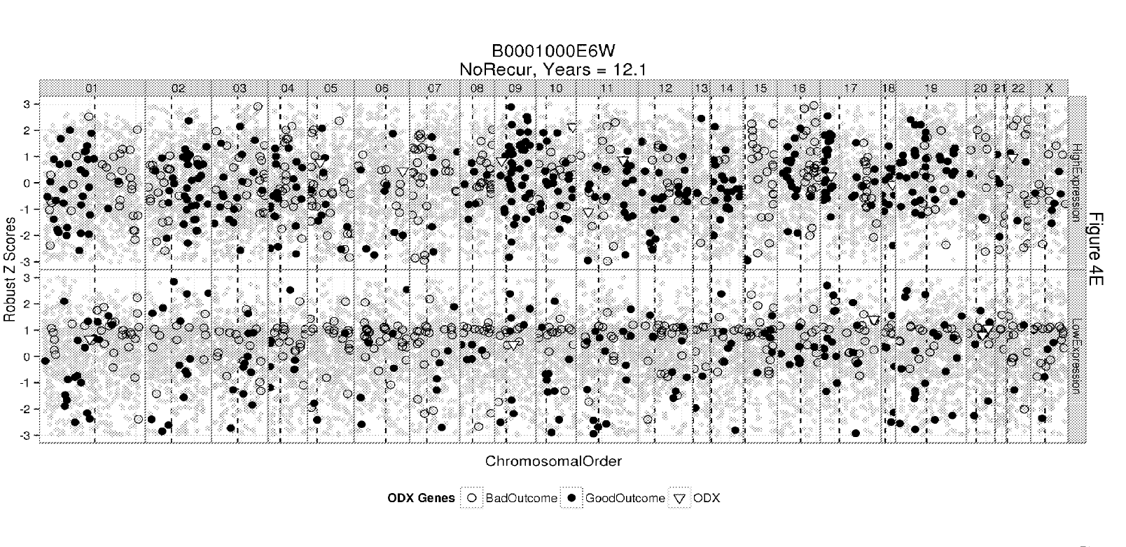

The effect sizes and statistical significance of Oncotype DX® genes were similar when screening was carried out by whole transcriptome RNA-Seq rather than RT-PCR (compare FIGS. 1A and 1B of PCT/US2012/063313, filed Nov. 2, 2012, now WO 2013/070521)). This is shown in detail on a gene by gene basis in box plots (see FIG. 2 of PCT/US2012/063313, filed Nov. 2, 2012, now WO 2013/070521). A scatter plot of log hazard ratios demonstrates overall concordance between the 192 gene RT-PCR results with the RNA-Seq analyses (Lin et al., Journal of the Royal Statistical Society, Series B 84, 1074 (1989)) (Lin concordance correlation: 0.810; Pearson correlation coefficient: 0.813; see FIG. 3 of PCT/US2012/063313, filed Nov. 2, 2012, now WO 2013/070521). Significantly, RNA-Seq further associates many RefSeq RNAs with disease recurrence: a total of 1307 at FDR<10% (see PCT/US2012/063313, filed Nov. 2, 2012, now WO 2013/070521), hereafter referred to as “identified RefSeq RNAs.” In contrast, the 192 gene RT-PCR study identified 32 RNAs at FDR<10%, and consumed five-fold more input RNA. Together, these results indicate that RNA-Seq can provide a practical, sensitive and precise platform for genome-wide biomarker discovery in FFPE tissue.

TABLE 1
Case characteristics and outcomes

| | |
|---|---|
| | No. patients/no. analyzed |
| Characteristic | (%) |

| | | |
|---|---|---|
| Tumor size (cm) | | |
| 0-2 | 81/136 | (60%) |
| 2-5 | 49/136 | (36%) |
| >5 | 6/136 | (4%) |
| No. lymph nodes at primary diagnosis | | |
| 0 | 110/136 | (81%) |
| 1-9 | 11/136 | (8%) |
| 10-15 | 9/136 | (7%) |
| 16-20 | 6/136 | (4%) |
| Adjuvant tamoxifen | | |
| Yes | 54/136 | (40%) |
| No | 77/136 | (57%) |
| Unknown | 5/136 | (4%) |
| Adjuvant chemotherapy | | |
| Yes | 51/136 | (38%) |
| No | 79/136 | (58%) |
| Unknown | 6/136 | (4%) |
| ER Status* | | |
| ER positive | 111/136 | (82%) |
| ER negative | 25/136 | (18%) |
| Vital Status | | |
| Distant recurrence, death due to breast | | |
| cancer, or death due to unknown cause | | |
| Total | 26/136 | (19%) |
| ER positive | 16/136 | (12%) |
| ER negative | 10/136 | (7%) |
| Alive without distant recurrence or | | |
| death due to other cause | | |
| Total | 110/136 | (81%) |
| ER positive | 95/136 | (70%) |
| ER negative | 15/136 | (11%) |

*ER status determined by RNA-Seq analysis as described

### Example 3

### Mapped Strings

### Gene Exon Analysis

A total of 1307 tumor mRNA exonic species associate with breast cancer distant disease recurrence at a false discovery rate of 10% in the recently published RNA-Seq study of the 136 patient Providence cohort, roughly equal numbers associated with increased risk of recurrence (standardized hazard ratio (SHR)>1) and decreased risk of recurrence (SHR<1) (Sinicropi et al. PLoS One e7: 40092, 2012). FIG. 1A places the identified 1307 genes on a physical map of the human genome, demonstrating that there are physical gaps, and often large ones, between nearest neighbors in a string. That is, many genes that were not identified at FDR 10% as being associated with disease recurrence risk are often located between nearest neighbors in a string. Visual inspection of FIG. 1A, for example, suggests that that these two classes of risk-associated genes tend to be non-randomly distributed in strings across the genome, an impression that is confirmed by statistical analysis: The likelihood of the longest string (35 genes on chromosome 7p, associated with increased risk) occurring by chance is very low (p=2×10⁻⁷).

In the Providence RNA-Seq dataset 111 of the 136 patients were designated as ER+, and in these cases 363 genes were identified as statistically associated with recurrence risk (Sinicropi et al. PLoS One 7: e40092, 2012). As shown in FIG. 1B, the distribution of chromosomal locations of these identified genes was again distinctly nonrandom. For example, of the 20 identified genes located on chromosome 16q, 19 have HR>1; a string of 20 genes with HR<1 is located on chromosome 1p (p=0.005).

We next asked whether mapped string distribution patterns occur in an independent breast cancer dataset, using results from the NM DNA-microarray gene expression study of 319 breast cancer patients (Van't Veer L J, et al. Nature 415: 530-536, 2002). Normalized expression values for 13120 genes are evaluable in this dataset. Using the same statistical approach as applied to the normalized Providence gene expression data (Sinicropi et al PLoS One e7: 40092, 2012), we associated 2032 genes with disease-free survival in the NM dataset. These results are roughly in accord with those from other analyses of the NKI dataset. This list of discovered genes contains 42 of those in the NKI 70 gene panel and 11 in the Genomic Health, Inc. 21 gene panel. Mapping these 2032 genes on their chromosomal coordinates reveals many long strings of identified genes FIG. 2. There are 110 strings containing 5 or more genes. The likelihood of longest of these, 50 genes in a row with HR>1 on chromosome 16, occurring by chance is very low (p=1.28×10⁻¹¹). (Data not shown). Thus, mapped strings of genes associated with good or bad prognosis are a feature of datasets developed with independent cohorts of breast cancer patients and different technology platforms.

Mapped strings create metagenes that can be explored for biomarker activity. To do this we defined a string metagene as a mapped string of 5 or more genes, the upper limit of which is bounded by either the end of the string (where HR>1 flips to HR<1 or vice versa) or the end of a chromosome arm. This yields a total of 59 strings in the Providence dataset (all patients). The 59 mapped strings identified in the Providence dataset are shown in Table 2A. Not surprisingly, the unadjusted p-values for the strings are low, ranging from 5×10-8 to 2×10⁻¹³ for the top 15 strings (Table 2A). More interestingly, the effect sizes (absolute standardized log hazard ratios corrected for regression to the mean, RMC-ASLHR) are notably high: the 15 top metagene RMC-ALSHR values ranged from 1.57 to 1.74. Mapped strings longer than 5 genes tended to have larger RMC-ALSHR values than any of the individual genes in the string, in analyses of either all patients or ER+ patients (FIG. 3A and FIG. 3B). These results from the Providence RNA-Seq dataset suggest that metagenes formed from mapped strings merit exploration as candidate prognostic biomarkers in breast cancer.

Because ultimately the clinical significance of a biomarker depends on its sustained performance across independent patient populations, we evaluated the prognostic performance of the 59 mapped strings from the Providence dataset in the NM patient breast cancer dataset. It was not possible to do this precisely because a number of the genes that belong to 59 Providence strings were not identified in the NKI dataset, so the strings that we evaluated in the latter dataset were diminished by 208 genes, out of a starting total of 636. Despite this technical problem (7 of the strings evaluated in NKI contained 3 or fewer genes), 19 strings were statistically significantly associated with the DFS endpoint in the NM cohort (Bonferroni-adjusted p<0.05), with RMC-ALSHR ranging from 1.20 to 1.34; see Tables 4 and 5 for additional data). Only one of these 19 strings that associated with DFS had a string length less than 5 in the NM data set. In contrast, of the 19 strings with weakest statistical association with DFS, 12 contained fewer than 5 genes in the NM dataset. The results with the DFS clinical endpoint were substantially weaker than those obtained with the OS clinical endpoint and slightly stronger than those obtained with the MFS clinical endpoint.

TABLE 3
Performance of 59 Providence Strings in the NKI dataset

| | Fraction of Strings That Were |
|---|---|
| | Statistically Significantly* |
| Endpoint | Associated with Endpoint |
| Metastasis-Free Survival | 16/59 (27%) |
| Disease-Free Survival | 19/59 (32%) |
| Overall Survival | 23/59 (39%) |

*Bonferroni-adjusted p-value < 0.05.

TABLE 4
Top Providence Strings for Association with Progression-
free Survival in NKI cohort/dataset

| | String (identified by starting coordinate) | | |
|---|---|---|---|
| | 42,295,709 | 63,953,587 | 612,405 |
| Chromosome arm | 20q | 11q | 5p |
| String length Providence | 7 | 11 | 6 |
| dataset | | | |
| String length NKI dataset | 7 | 7 | 5 |
| Direction of prognosis | HR > 1 | HR > 1 | HR > 1 |
| P-value | 7 × 10⁻⁸ | 6 × 10⁻⁸ | 1.5 × 10⁻⁷ |
| Bonferroni-corrected | 4 × 10⁻⁶ | 4 × 10⁻⁶ | 8 × 10⁻⁶ |
| p-value | | | |
| RM-Corrected Standardized | 1.34 | 1.34 | 1.32 |
| Hazard Ratio (95% Conf. | | | |
| Int.) | | | |

Additional mapped strings were mainly generated by enhancing the 59 mapped strings from the Providence dataset (described above) with neighboring exonic species from the 1307 tumor mRNA exonic species found to associate in the same direction with breast cancer distant disease recurrence at a false discovery rate of 10%. They were also enhanced by adding genes from other chromosomes that strongly co-expressed, for example, the proliferation genes or a subset of mapped strings that coexpressed with G3BP2. Those 1307 tumor mRNA exonic species are described above and in Sinicropi et al. PLoS One e7: 40092, 2012. Table 2B shows data from all patients in the above-described Providence patient cohort; Table 2C shows data from ER-positive in the above-referenced Providence patient cohort. These data include the exonic species present in the enhanced mapped strings, the p-value, and the RM-corrected absolute standardized hazard ratio (95% confidence interval). Notes concerning various strings in Tables 2B and 2C are identified with a superscript letter and described below the given table.

### Example 4

### Mapped Strings

### Intronic Analysis

Intron expression within strings was also assessed for their relationship with recurrence of breast cancer. This analysis is performed exactly as the mapped string analysis carried out for gene exons (see Example 3, above), except performed on intronic sequences that had been associated on a univariate intron by intron basis with breast cancer recurrence risk (see Sinicropi D. et al. PLoS One e7: 40092, 2012).

Table 5A shows 79 mapped strings based on identified introns from the Providence dataset, and Table 5B shows the accession no. and exemplary location of introns in strings based on introns identified in the Providence dataset.

### Example 5

### Mapped Strings

### Whole Gene Analysis

Mapped strings based on whole gene (exons and introns) analysis were assessed for their relationship with recurrence of breast cancer. This analysis is performed exactly as the mapped string analysis carried out for gene exons (see Example 3, above), except performed on complete gene (exonic plus intronic) sequences that had been associated with breast cancer recurrence risk on a univariate gene by gene basis.

Table 6A shows 75 mapped strings from whole gene (exon+intron) analysis of the Providence dataset. Table 6B shows the gene accession numbers for the whole genes shown in Table 6A.

### Example 6

### Transcriptome Profile Maps

We are unable to explore the relationship between mapped strings and mapped DNA copy number alterations (CNAs) within the Providence or NKI datasets because tumor DNA is unavailable in both cases. Therefore, we sought an RNA surrogate for CNA information, reasoning that amplification of DNA in a chromosomal region would often result in RNA over-expression in that region.

Chromosomal DNA amplifications or deletions are straightforward to visualize against the diploid copy number background of the normal human genome. However, gene expression is widely variable from gene to gene. To develop a baseline against which RNA over- or under-expression could be visualized, at each gene locus we determined the average normalized expression of that gene. Then, for each patient tumor, at the locus of each coding gene we determined the difference in expressed RNA for that patient and the patient cohort average (measured by Z-scores), generating graphics that we call transcriptome profile maps (TPMs). Incorporating data from low abundance RNAs generates noise that can obscure TPM features. Therefore, for each patient/tumor we created two maps, for RNAs above or below the 50^{th} percentile in abundance (normalized counts).

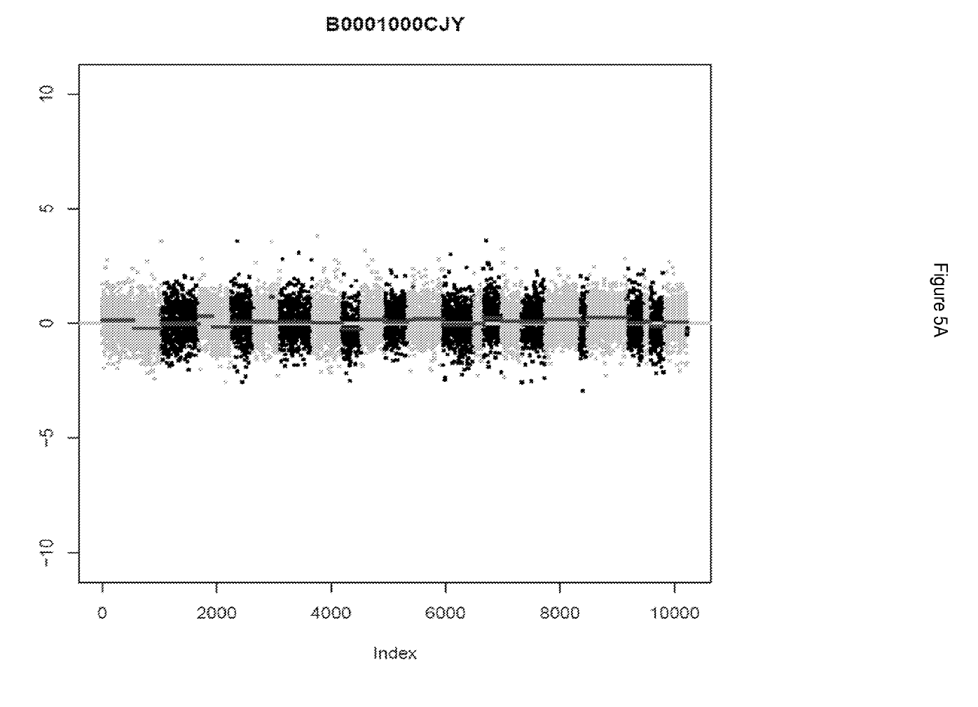

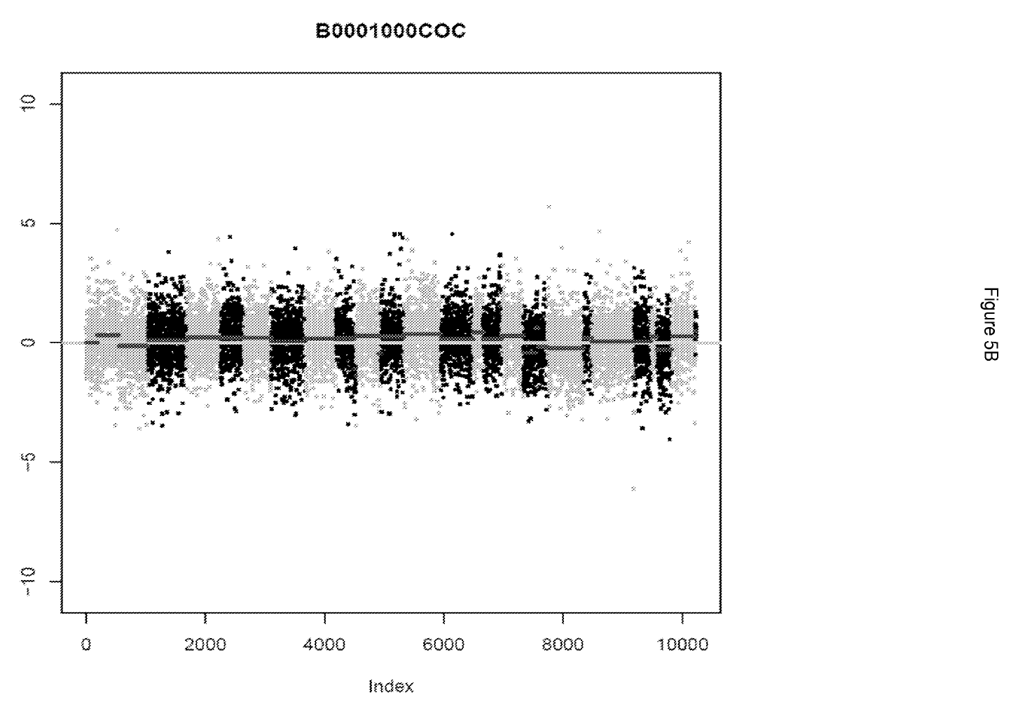

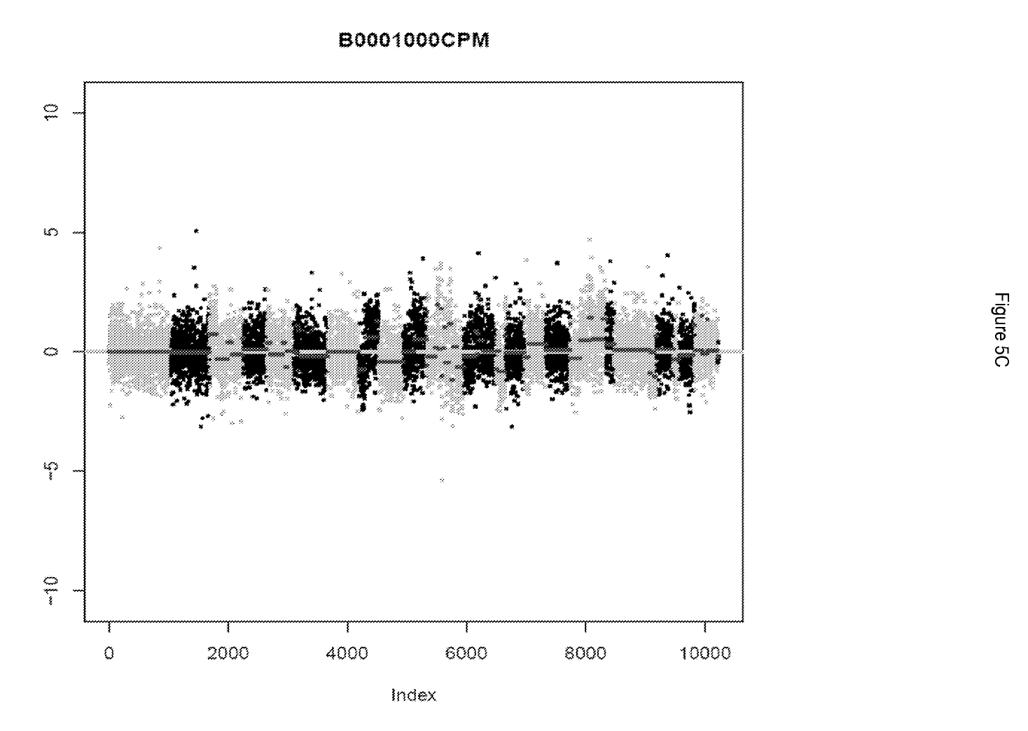

Examples of TPMs for 5 different Providence patient tumors, generated using the published RNA-Seq data (Sinicropi et al. PLoS One e7: 40092, 2012), are shown in FIG. 4 A-E. The TPMs for Providence patient tumors tend to be different from patient. They vary in global Z-score (Y-axis) dispersion (compare FIGS. 4 B, C and D). Further, many but not all TPMs exhibit Z-score (Y-axis) spikes that vary in number, width and magnitude (most evident in FIGS. 4 D and E). Variation in global dispersion and variation in spike features are not mutually exclusive. (For example, some TPMs exhibit few spikes and wide dispersion, others few spikes and narrow dispersion, or many spikes and wide dispersion, or many spikes and relatively narrow dispersion.) As shown in FIG. 4, and anticipated, TPM features tend to be more distinct in graphs of RNAs with abundance values greater than the 50^{th} percentile, relative to below the 50^{th} percentile. (It may be noted that concentrated data points form horizontal ribs across TPMs of the lower abundance RNAs. These represent RNAs having zero normalized counts.)

TPM features can be mined for candidate prognostic biomarkers. We asked whether any of three measures of TPM dispersion associate with the rate or breast cancer recurrence in the Providence dataset. Two of these, mean absolute deviation from median, and mean absolute deviation location to location associated with increased risk, at 1.62 (p=0.007) and 1.45 (p=0.018) SHRs, respectively (Table 7).

TABLE 7
Association of Global Dispersion Measures with
Risk of Recurrence from the Providence dataset

| | Standardized HR | |
|---|---|---|
| Global Dispersion Measure | (95% CI) | p-value |
| Proportion of Genes with | 1.35 (0.92, 1.97) | 0.12 |
| Expression < Q1 or > Q3 | | |
| Mean Absolute Deviation of Gene | 1.62 (1.14, 2.29) | 0.007 |
| Expression from Median | | |
| Mean Absolute Deviation Location | 1.45 (1.07, 2.04) | 0.018 |
| to Location | | |

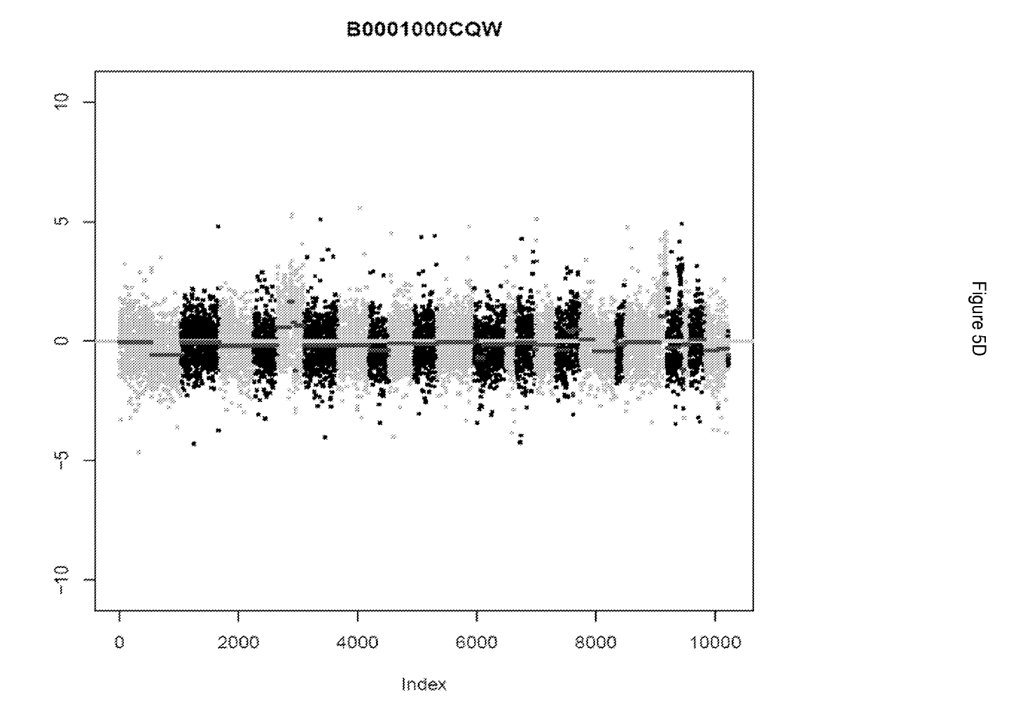

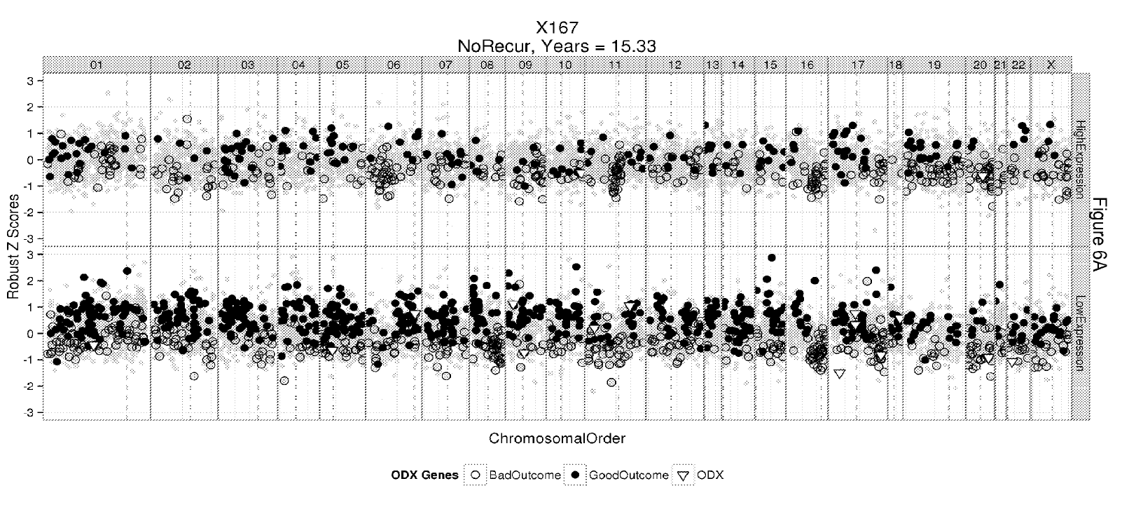

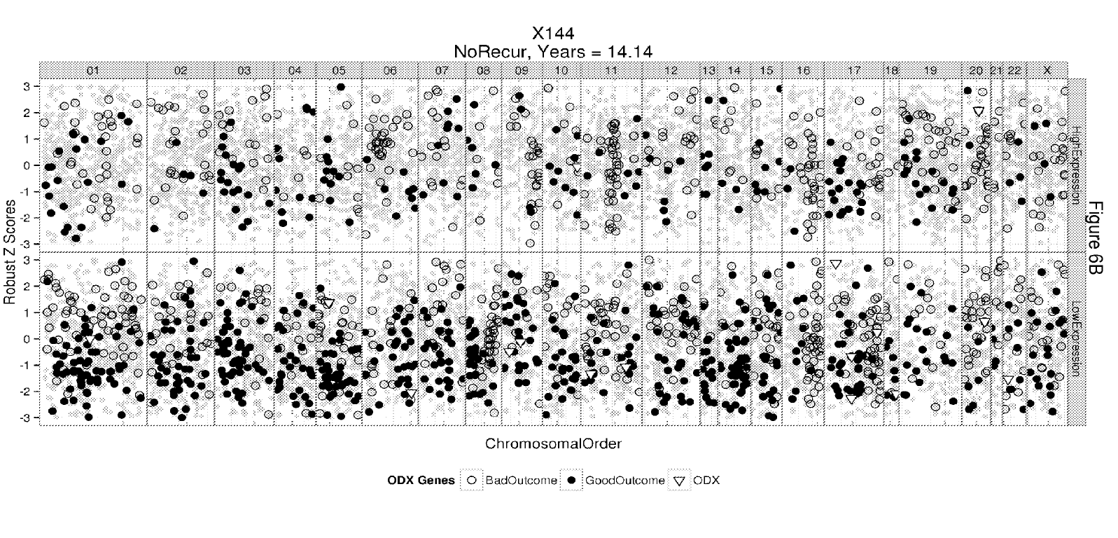

To determine whether the number of Z score spike features in TPMs associate with recurrence rate, we applied two leading segmentation algorithms: circular binary segmentation (CBS; Venkatraman E S and Olshen A B, Bioinformatics 23:657-63, 2007) and piecewise constant fitting (PCF; Nilsen G et al., BMC Genomics 13:591, 2012). For CBS we prescribed 5 as the lowest permitted number of genes that define a segment while the penalty parameter governing number of elicited segments for PCF was determined by cross-validation. Results from the two approaches exhibit good agreement (correlation 0.74) as do downstream analyses relating numbers of segments (cf the genomic instability index employed in Bilal et al., PLoS Comput Biol 9:e1003047, 2013). We present results based on PCF. Examples of TPM segment plots, derived from Providence RNA-Seq data, are shown in FIG. 5 A-D. The number of TPM segments present in Providence patient breast cancers relates to the rate of distant recurrence of the disease (SHR=1.5; p=0.019); Table 8.

TABLE 8
Association between segment number and incidence
of recurrence in Providence and NKI datasets

| Data Set | Endpoint | SHR | Wald P-value |
|---|---|---|---|
| Providence | RFS | 1.5 | 0.019 |
| NKI | DFS | 1.4 | 7 × 10⁻⁵ |
| | MFS | 1.4 | 7 × 10⁻⁴ |
| | OS | 1.8 | 2 × 10⁻⁷ |

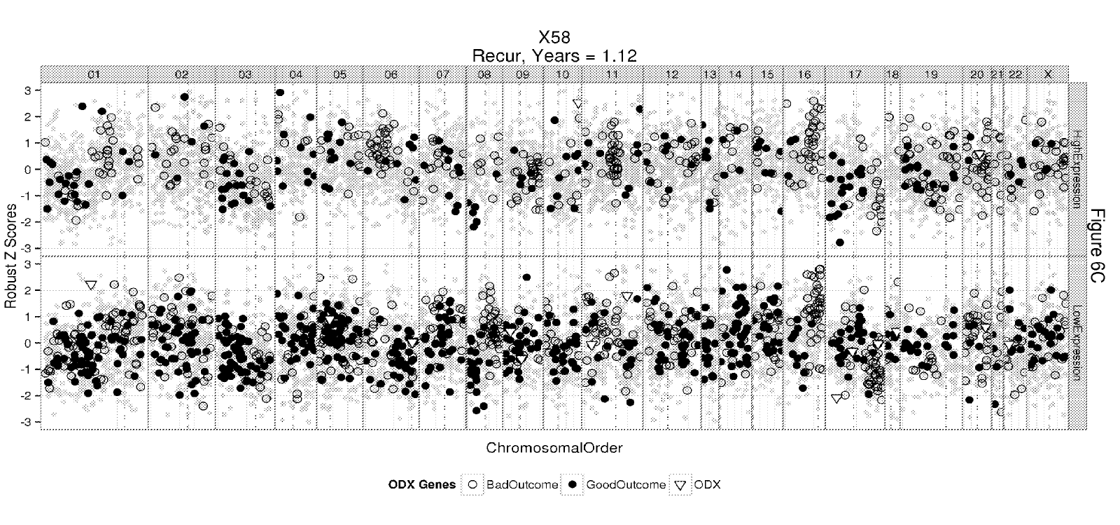

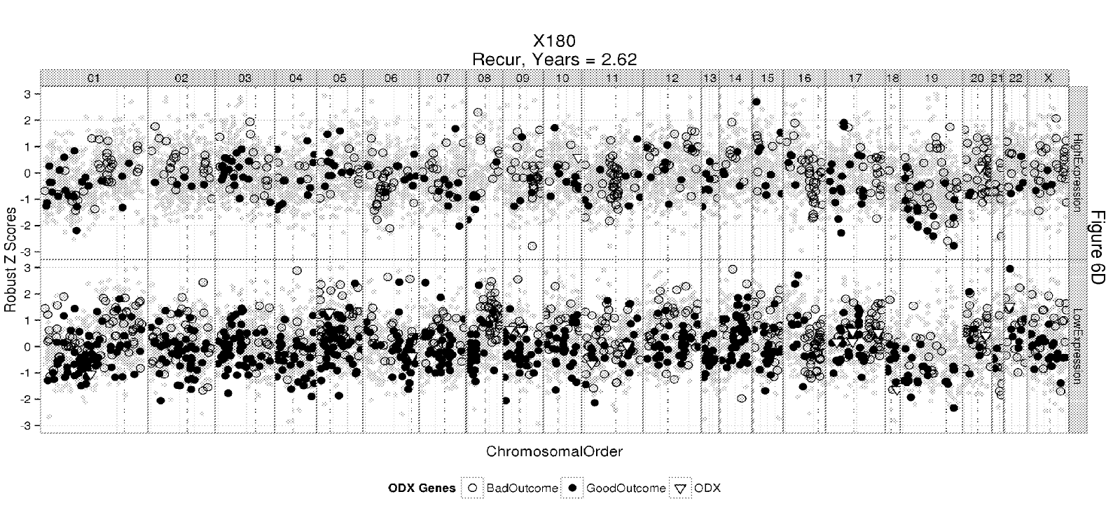

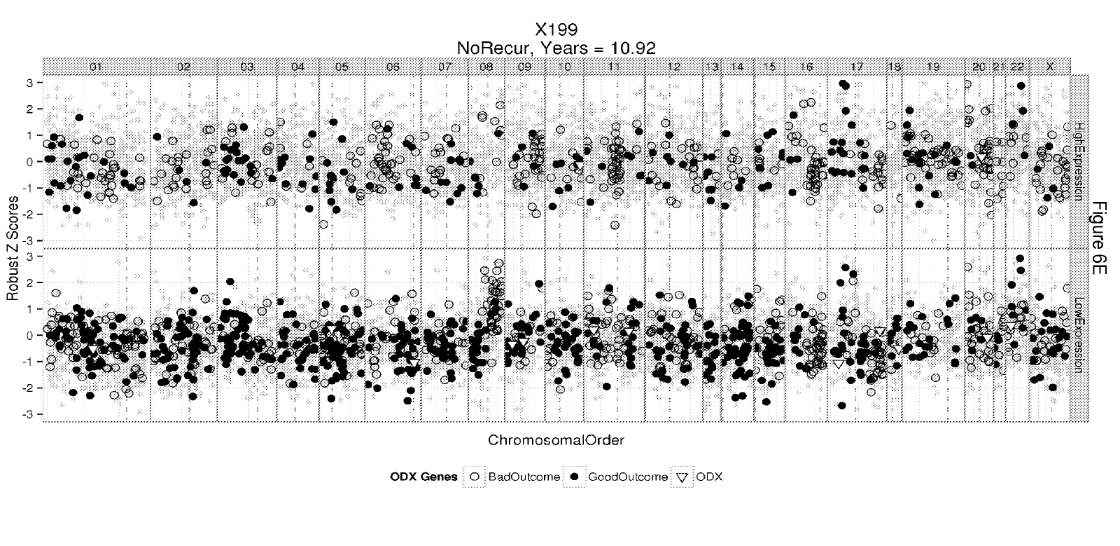

Next, we developed TPMs from NKI cohort breast cancer data. Because mRNA quantification in this case is based on DNA microarray technology, we modified the protocol for producing TPMs accordingly. We did not attempt to filter data based on detected mRNA abundance. We did filter data based on variance of expression of mRNAs. TPM graphs generated with mRNAs having greatest variability inexpression (top 50^{th} percentile) tended to have more clearly delineated features (for example, see FIGS. 6 A-E). To a first approximation, these TPMs visually resemble the Providence cohort breast cancer (RNA-Seq-based) TPMs (compare FIGS. 4 and 6). NKI patient/tumor TPMs vary widely in Z-score global dispersion. Furthermore, Z score spikes are evident in many specimens, and these most frequently occur in the same chromosomal arms as in Providence patient/tumor TPMs, namely on chromosomes 8, 11, 16 and 17.

As observed in the Providence dataset, TPM segment counts strongly relate to all three measures of disease recurrence in this dataset (Table 8): for example, with respect to disease-free survival SHR=1.4 with Wald p-value=6×10⁻⁵. (PCF: SHR=1.5, p=2×10⁻⁶). In contrast to the results in the Providence dataset, measures of Z-score global dispersion do not associate with outcome in the NM dataset (Table 7).

All references cited throughout the disclosure, including the examples, are hereby expressly incorporated by reference for their entire disclosure.

While the present invention has been described with reference to what is considered to be specific embodiments, it is to be understood that the invention is not so limited. To the contrary, the invention is intended to cover various modifications and equivalents included within the spirit and scope of the appended claims.

TABLE 2A
59 Strings Identified in the Providence Dataset (exon analysis)

| | | | | | | | | | | RM-Corrected Ab- |
|---|---|---|---|---|---|---|---|---|---|---|
| | | | | | Exemplary Start | Exemplary End | | | | solute Standard- |
| String | Genes | Gene Accession Nos. | Chrom. | | Location of | Location of | # Genes | Dir. of | | ized Hazard Ratio |
| No. | in Run | of Genes in Run | (23 = X, 24 = Y) | Arm | First Gene in Run | Last Gene in Run | in Run | Association | p-value | (95% Conf. Int.) |
| 1 | TTC4 | NM_004623 NM_001004303 NM_002227 | 1 | p | 55,181,495 | 90,494,094 | 9 | −1 | 6.66E−15 | 1.87 |
| | C1orf168 | NM_001126044, NM_198716, NM_198718, | | | | | | | | (1.60, 2.18) |
| | JAK1 | NM_198715, NM_198719, NM_198714, | | | | | | | | |
| | PTGER3 | NR_028292, NR_028293, NM_198717, | | | | | | | | |
| | NEGR1 | NR_028294 NM_173808 NM_001002912 | | | | | | | | |
| | C1orf173 | NM_001130042, NM_001130043, NM_001889, | | | | | | | | |
| | CRYZ | NM_001134759 NM_025065 NM_182976, | | | | | | | | |
| | RPF1 | NM_182975 | | | | | | | | |
| | ZNF326 | | | | | | | | | |
| 2 | PCDP1 | NM_001029996 NM_001161415, NM_001161417, | 2 | q | 120,302,025 | 175,294,303 | 18 | −1 | 3.44E−12 | 1.76 |
| | GPR17 | NM_001161416, NM_005291 NM_001024074, | | | | | | | | (1.50, 2.07) |
| | HNMT | NM_006895, NM_001024075 NM_004522 | | | | | | | | |
| | KIF5C | NM_177964 NM_014568 NM_013450 | | | | | | | | |
| | LYPD6B | NM_022058, NM_001178016, NM_001178015 | | | | | | | | |
| | GALNT5 | NM_001171631, NM_052946, NM_001039724, | | | | | | | | |
| | BAZ2B | NM_001171632 NM_004525 NM_152384 | | | | | | | | |
| | SLC4A10 | NM_006063 NM_004792 NM_001085447 | | | | | | | | |
| | NOSTRIN | NM_001008489, NM_001199287, NM_001199288, | | | | | | | | |
| | LRP2 | NM_001199285, NM_001199286 NM_001127383, | | | | | | | | |
| | BBS5 | NM_024843 NM_003705 NM_024583, | | | | | | | | |
| | KBTBD10 | NM_001193528 | | | | | | | | |
| | PPIG | | | | | | | | | |
| | C2orf77 | | | | | | | | | |
| | PHOSPHO2 | | | | | | | | | |
| | CYBRD1 | | | | | | | | | |
| | SLC25A12 | | | | | | | | | |
| | SCRN3 | | | | | | | | | |
| 3 | OR9K2 | NM_001005243 NM_152637 NM_015257, | 12 | q | 55,523,553 | 72,426,221 | 8 | 1 | 1.42E−13 | 1.74 |
| | METTL7B | NM_001130963 NM_007224 NM_001166359, | | | | | | | | (1.50, 2.01) |
| | TMEM194A | NM_001166358, NM_001166357, NM_001166356, | | | | | | | | |
| | NXPH4 | NM_005412, NR_029416, NR_029417, | | | | | | | | |
| | SHMT2 | NR_029415 NM_001136051, NM_153377 | | | | | | | | |
| | LRIG3 | NM_007235 NM_173353 | | | | | | | | |
| | XPOT | | | | | | | | | |
| | TPH2 | | | | | | | | | |
| 4 | YWHAZ | NM_001135699, NM_001135701, NM_001135702, | 8 | q | 101,930,804 | 126,034,525 | 6 | 1 | 1.60E−11 | 1.74 |
| | GRHL2 | NM_001135700, NM_145690, NM_003406 | | | | | | | | (1.48, 2.04) |
| | LRP12 | NM_024915 NM_001135703, NM_013437 | | | | | | | | |
| | KCNV1 | NM_014379 NR_024479 NM_003129 | | | | | | | | |
| | LOC100131726 | | | | | | | | | |
| | SQLE | | | | | | | | | |
| 5 | SFXN1 | NM_022754 NM_032361 NM_014613 | 5 | q | 174,905,514 | 179,265,077 | 13 | 1 | 1.66E−11 | 1.74 |
| | THOC3 | NM_052899 NM_001001502, NM_003085 | | | | | | | | (1.48, 2.04) |
| | FAF2 | NM_012171, NM_130465, NM_001006616 | | | | | | | | |
| | GPRIN1 | NM_030567, NM_001174101, NM_001174102 | | | | | | | | |
| | SNCB | NM_017510 NR_003281 NR_024035 | | | | | | | | |
| | TSPAN17 | NM_001746, NM_001024649 NM_014275, | | | | | | | | |
| | PRR7 | NM_054013 NM_003900, NM_001142299, | | | | | | | | |
| | TMED9 | NM_001142298 | | | | | | | | |
| | GMCL1L | | | | | | | | | |
| | AACSP1 | | | | | | | | | |
| | CANX | | | | | | | | | |
| | MGAT4B | | | | | | | | | |
| | SQSTM1 | | | | | | | | | |
| 6 | LIX1 | NM_153234 NR_000039 NM_001962 | 5 | q | 96,427,574 | 148,734,146 | 12 | 1 | 3.78E−11 | 1.72 |
| | RAB9BP1 | NM_177478 NM_130809 NM_003374, | | | | | | | | (1.47, 2.02) |
| | EFNA5 | NR_036625, NR_036624 NM_021982 | | | | | | | | |
| | FTMT | NM_001167577, NM_020389, NM_001167576 | | | | | | | | |
| | PRRC1 | NM_005733 NM_022809, NM_001790 | | | | | | | | |
| | VDAC1 | NM_005219, NM_001079812 NM_152407 | | | | | | | | |
| | SEC24A | | | | | | | | | |
| | TRPC7 | | | | | | | | | |
| | KIF20A | | | | | | | | | |
| | CDC25C | | | | | | | | | |
| | DIAPH1 | | | | | | | | | |
| | GRPEL2 | | | | | | | | | |
| 7 | PCSK5 | NM_006200, NM_001190482 NM_001135953, | 9 | q | 78,505,560 | 108,153,682 | 27 | −1 | 3.91E−11 | 1.71 |
| | GKAP1 | NM_025211 NM_017576 NM_032307 | | | | | | | | (1.46, 2.00) |
| | KIF27 | NM_024635 NM_001170639, NM_178432, | | | | | | | | |
| | C9orf64 | NM_001170640, NM_001039803, NM_012119 | | | | | | | | |
| | NAA35 | NM_006717 NM_001161625, NM_145283 | | | | | | | | |
| | CDK20 | NM_017948, NR_024020 NM_001012267 | | | | | | | | |
| | SPIN1 | NM_014057, NM_033014 NM_005014 | | | | | | | | |
| | NXNL2 | NM_017680, NM_001193335 NM_001393, | | | | | | | | |
| | NOL8 | NM_001197295, NM_001197296 NM_001003800, | | | | | | | | |
| | CENPP | NM_015250 NM_001007101, NM_031486 | | | | | | | | |
| | OGN | NM_001083536, NM_033086 NM_145006 | | | | | | | | |
| | OMD | NM_198841 NM_005392 NM_000507, | | | | | | | | |
| | ASPN | NM_001127628 NM_001193329, NM_001193331, | | | | | | | | |
| | ECM2 | NM_032823 NM_007001 NM_001001662 | | | | | | | | |
| | BICD2 | NR_036526 NM_016481 NM_080546 | | | | | | | | |
| | ZNF484 | | | | | | | | | |
| | FGD3 | | | | | | | | | |
| | SUSD3 | | | | | | | | | |
| | FAM120AOS | | | | | | | | | |
| | PHF2 | | | | | | | | | |
| | FBP1 | | | | | | | | | |
| | C9orf3 | | | | | | | | | |
| | SLC35D2 | | | | | | | | | |
| | ZNF782 | | | | | | | | | |
| | LOC100499484 | | | | | | | | | |
| | C9orf156 | | | | | | | | | |
| | SLC44A1 | | | | | | | | | |
| 8 | MIR196A1 | NR_029582 NM_006361 NM_006546, | 17 | q | 46,709,852 | 54,560,007 | 7 | 1 | 1.28E−14 | 1.70 |
| | HOXB13 | NM_001160423 NM_002634 NM_000269, | | | | | | | | (1.49, 1.95) |
| | IGF2BP1 | NM_198175 NM_001102402, NM_021213 | | | | | | | | |
| | PHB | NM_153228 | | | | | | | | |
| | NME1 | | | | | | | | | |
| | PCTP | | | | | | | | | |
| | ANKFN1 | | | | | | | | | |
| 9 | ASB13 | NM_024701, NR_037164, NR_024581 | 10 | p | 5,680,820 | 37,521,495 | 8 | −1 | 3.65E−11 | 1.68 |
| | GATA3 | NM_001002295, NM_002051 NM_031455 | | | | | | | | (1.44, 1.97) |
| | CCDC3 | NM_001004470 NM_001098500, NM_019590, | | | | | | | | |
| | ST8SIA6 | NM_001098501 NM_001143771, NM_001143767, | | | | | | | | |
| | KIAA1217 | NM_001143766, NM_001143770, NM_182755, | | | | | | | | |
| | ZNF438 | NR_026560, NM_001143768, NM_001143769 | | | | | | | | |
| | C10orf68 | NM_024688 NM_052997 | | | | | | | | |
| | ANKRD30A | | | | | | | | | |
| 10 | TPD52L1 | NM_003287, NM_001003397, NM_001003396, | 6 | q | 125,474,879 | 150,725,765 | 10 | 1 | 2.90E−10 | 1.67 |
| | OR2A4 | NM_001003395 NM_030908 NM_175067 | | | | | | | | (1.42, 1.95) |
| | TAAR6 | NM_022121 NM_020464, NM_001144060 | | | | | | | | |
| | PERP | NM_016485 NM_198569, NM_020455, | | | | | | | | |
| | NHSL1 | NM_001032395, NM_001032394 NR_024045 | | | | | | | | |
| | VTA1 | NM_024518 NM_001164694, NM_001164695, | | | | | | | | |
| | GPR126 | NM_203395 | | | | | | | | |
| | RAET1K | | | | | | | | | |
| | ULBP3 | | | | | | | | | |
| | IYD | | | | | | | | | |
| 11 | UNC119B | NM_001080533 NM_002560 NM_001194995, | 12 | q | 121,147,831 | 124,192,948 | 6 | −1 | 2.05E−10 | 1.67 |
| | P2RX4 | NM_001143905, NM_152269 NM_145058 | | | | | | | | (1.42, 1.95) |
| | C12orf65 | NM_178314 NM_024809, NM_001143850 | | | | | | | | |
| | RILPL2 | | | | | | | | | |
| | RILPL1 | | | | | | | | | |
| | TCTN2 | | | | | | | | | |
| 12 | PDE4A | NM_001111308, NM_001111307, NM_006202, | 19 | p | 10,531,333 | 12,662,356 | 15 | −1 | 1.09E−11 | 1.65 |
| | ZNF653 | NM_001111309 NM_138783 NM_001080493 | | | | | | | | (1.43, 1.91) |
| | ZNF823 | NM_152355 NM_152357 NM_001012753 | | | | | | | | |
| | ZNF441 | NM_001080411 NM_001136501 NR_027049 | | | | | | | | |
| | ZNF440 | NM_021143 NM_145233 NM_016264, | | | | | | | | |
| | ZNF763 | NM_001164276 NM_145276 NM_030824 | | | | | | | | |
| | ZNF433 | NM_144976 | | | | | | | | |
| | ZNF844 | | | | | | | | | |
| | ZNF788 | | | | | | | | | |
| | ZNF20 | | | | | | | | | |
| | ZNF625 | | | | | | | | | |
| | ZNF44 | | | | | | | | | |
| | ZNF563 | | | | | | | | | |
| | ZNF442 | | | | | | | | | |
| | ZNF564 | | | | | | | | | |
| 13 | STAT5A | NM_003152 NM_016437 NM_016602 | 17 | q | 40,439,565 | 42,980,629 | 6 | −1 | 7.81E−12 | 1.65 |
| | TUBG2 | NR_024462, NR_024461 NM_032376 | | | | | | | | (1.43, 1.91) |
| | CCR10 | NM_213607 | | | | | | | | |
| | LOC100190938 | | | | | | | | | |
| | TMEM101 | | | | | | | | | |
| | CCDC103 | | | | | | | | | |
| 14 | ZNF141 | NM_003441 NR_002451 NM_001145291, | 4 | p | 331,596 | 8,442,452 | 9 | −1 | 3.77E−10 | 1.64 |
| | ABCA11P | NM_001145292, NM_000283 NM_032219 | | | | | | | | (1.41, 1.92) |
| | PDE6B | NM_006315 NM_006651 NR_033768 | | | | | | | | |
| | MFSD7 | NM_018401 NM_003501, NM_001101667 | | | | | | | | |
| | PCGF3 | | | | | | | | | |
| | CPLX1 | | | | | | | | | |
| | TMED11P | | | | | | | | | |
| | STK32B | | | | | | | | | |
| | ACOX3 | | | | | | | | | |
| 15 | CAV3 | NM_033337, NM_001234 NM_001018115, | 3 | p | 8,775,486 | 20,227,724 | 6 | 1 | 2.23E−09 | 1.64 |
| | FANCD2 | NM_033084 NR_024273, NM_001136232, | | | | | | | | (1.39, 1.92) |
| | SEC13 | NR_024272, NM_183352 NM_018306 | | | | | | | | |
| | TMEM40 | NR_027927 NM_001012411, NM_001199253, | | | | | | | | |
| | COL6A4P1 | NM_001199252, NM_001199251, NM_001012412, | | | | | | | | |
| | SGOL1 | NM_001012409, NM_001199254, NM_001012410, | | | | | | | | |
| | | NM_138484, NM_001199256, NM_001199257, | | | | | | | | |
| | | NM_001012413, NM_001199255 | | | | | | | | |
| 16 | MFGE8 | NM_001114614, NM_005928 NR_015411 | 15 | q | 89,441,914 | 99,057,611 | 12 | 1 | 1.70E−13 | 1.63 |
| | LOC254559 | NM_016321 NM_152259 NM_002168 | | | | | | | | (1.43, 1.86) |
| | RHCG | NM_198925, NM_020210 NM_006384 | | | | | | | | |
| | C15orf42 | NM_001033088, NR_028052 NM_000057 | | | | | | | | |
| | IDH2 | NM_002569 NM_003981, NM_199414, | | | | | | | | |
| | SEMA4B | NM_199413 NM_182562 | | | | | | | | |
| | CIB1 | | | | | | | | | |
| | NGRN | | | | | | | | | |
| | BLM | | | | | | | | | |
| | FURIN | | | | | | | | | |
| | PRC1 | | | | | | | | | |
| | FAM169B | | | | | | | | | |
| 17 | BRDT | NM_001726, NM_207189 NM_024813 | 1 | p | 92,414,928 | 109,584,850 | 8 | −1 | 2.41E−09 | 1.63 |
| | RPAP2 | NR_034091 NM_019083 NM_144620 | | | | | | | | (1.39, 1.91) |
| | LOC100129046 | NM_020978 NR_024438 NM_001142551, | | | | | | | | |
| | CCDC76 | NM_001142550, NM_014969 | | | | | | | | |
| | LRRC39 | | | | | | | | | |
| | AMY2B | | | | | | | | | |
| | LOC648740 | | | | | | | | | |
| | WDR47 | | | | | | | | | |
| 18 | TIGD7 | NM_033208 NM_001032292, NM_003450 | 16 | p | 3,348,808 | 18,937,726 | 10 | −1 | 3.63E−09 | 1.62 |
| | ZNF174 | NM_015041, NM_024793 NM_004380, | | | | | | | | (1.38, 1.90) |
| | CLUAP1 | NM_001079846 NM_032575 NM_001079512 | | | | | | | | |
| | CREBBP | NM_001080530 NM_014048 NM_001184998, | | | | | | | | |
| | GLIS2 | NM_014647, NM_001184999 NM_015092 | | | | | | | | |
| | FAM18A | | | | | | | | | |
| | SNX29 | | | | | | | | | |
| | MKL2 | | | | | | | | | |
| | KIAA0430 | | | | | | | | | |
| | SMG1 | | | | | | | | | |
| 19 | ASTN2 | NM_001184735, NM_198188, NM_001184734, | 9 | q | 119,187,504 | 135,820,020 | 18 | −1 | 7.64E−09 | 1.61 |
| | CDK5RAP2 | NM_014010, NM_198187, NM_198186 | | | | | | | | (1.37, 1.89) |
| | MEGF9C5 | NM_001011649, NM_018249 NM_001080497 | | | | | | | | |
| | ZBTB26 | NM_001735 NM_020924 NM_012197 | | | | | | | | |
| | RABGAP1 | NR_026677 NM_002077 NM_001012502 | | | | | | | | |
| | NCRNA00287 | NM_203305, NM_001035254 NM_016035 | | | | | | | | |
| | GOLGA1 | NM_003127, NM_001130438, NM_001195532 | | | | | | | | |
| | C9orf117 | NM_006336 NM_020145 NM_016520 | | | | | | | | |
| | FAM102A | NM_013318 NM_007344 NM_001162427, | | | | | | | | |
| | COQ4 | NM_000368, NM_001162426 | | | | | | | | |
| | SPTAN1 | | | | | | | | | |
| | ZER1 | | | | | | | | | |
| | SH3GLB2 | | | | | | | | | |
| | C9orf78 | | | | | | | | | |
| | PRRC2B | | | | | | | | | |
| | TTF1 | | | | | | | | | |
| | TSC1 | | | | | | | | | |
| 20 | LMAN1L | NM_021819 NM_001030005 NM_004255 | 15 | q | 75,105,194 | 82,338,361 | 10 | 1 | 5.88E−09 | 1.61 |
| | CPLX3 | NM_015492 NM_032808 NM_004378 | | | | | | | | (1.37, 1.88) |
| | COX5A | NM_001102667, NM_001102668, NM_002789 | | | | | | | | |
| | C15orf39 | NM_000745 NM_007364 NM_032246 | | | | | | | | |
| | LINGO1 | | | | | | | | | |
| | CRABP1 | | | | | | | | | |
| | PSMA4 | | | | | | | | | |
| | CHRNA5 | | | | | | | | | |
| | TMED3 | | | | | | | | | |
| | MEX3B | | | | | | | | | |
| 21 | RAB28 | NM_001017979, NM_001159601, NM_004249 | 4 | p | 13,369,347 | 26,756,918 | 7 | −1 | 7.81E−10 | 1.60 |
| | BOD1L | NM_148894 NM_001177381, NM_182646, | | | | | | | | (1.38, 1.86) |
| | CPEB2 | NM_001177384, NM_182485, NM_001177382, | | | | | | | | |
| | FAM184B | NM_001177383 NM_015688 NM_017741 | | | | | | | | |
| | DCAF16 | NM_001130727, NM_145048 NM_018317 | | | | | | | | |
| | PACRGL | | | | | | | | | |
| | TBC1D19 | | | | | | | | | |
| 22 | UBXN10 | NM_152376 NM_032409 NM_003196 | 1 | p | 20,512,578 | 43,720,029 | 19 | −1 | 3.59E−10 | 1.60 |
| | PINK1 | NM_020317 NM_183008, NM_145345, | | | | | | | | (1.38, 1.85) |
| | TCEA3 | NM_001077262 NM_015023 NR_033926 | | | | | | | | |
| | C1orf63 | NM_001856 NM_001703 NR_026850 | | | | | | | | |
| | UBXN11 | NM_030786, NM_001161708 NM_052998 | | | | | | | | |
| | WDTC1 | NM_005202 NM_145047, NM_206837 | | | | | | | | |
| | LOC644961 | NM_003462 NM_004468 NM_033044, | | | | | | | | |
| | COL16A1 | NM_012090 NM_024503, NM_001127714 | | | | | | | | |
| | BAI2 | NM_001195831, NM_001167965, NM_152498, | | | | | | | | |
| | MTMR9LP | NM_001167966, NR_030778 | | | | | | | | |
| | SYNC | | | | | | | | | |
| | ADC | | | | | | | | | |
| | COL8A2 | | | | | | | | | |
| | OSCP1 | | | | | | | | | |
| | DNALI1 | | | | | | | | | |
| | FHL3 | | | | | | | | | |
| | MACF1 | | | | | | | | | |
| | HIVEP3 | | | | | | | | | |
| | WDR65 | | | | | | | | | |
| 23 | CEP72 | NM_018140 NM_004237, NM_001166260 | 5 | p | 612,405 | 3,536,208 | 6 | 1 | 6.49E−09 | 1.59 |
| | TRIP13 | NM_030782 NM_024830 NM_004553 | | | | | | | | (1.36, 1.86) |
| | CLPTM1L | NR_033898 | | | | | | | | |
| | LPCAT1 | | | | | | | | | |
| | NDUFS6 | | | | | | | | | |
| | LOC285577 | | | | | | | | | |
| 24 | SHCBP1 | NM_024745 NM_007006 NM_014669 | 16 | q | 46,614,468 | 70,611,571 | 17 | 1 | 2.33E−08 | 1.58 |
| | NUDT21 | NM_020312 NM_001145774, NM_201524, | | | | | | | | (1.35, 1.86) |
| | NUP93 | NM_005682, NM_001145771, NM_001145770, | | | | | | | | |
| | COQ9 | NM_001145773, NM_001145772, NM_201525 | | | | | | | | |
| | GPR56 | NM_001135639, NM_001297 NM_002428 | | | | | | | | |
| | CNGB1 | NM_001896 NM_006141 NM_004594 | | | | | | | | |
| | MMP15 | NM_001191022, NM_006565 NM_020850 | | | | | | | | |
| | CSNK2A2 | NM_024939 NM_030579 NM_199424, | | | | | | | | |
| | DYNC1LI2 | NM_007014, NM_199423 NM_001605 | | | | | | | | |
| | SLC9A5 | NM_012426 | | | | | | | | |
| | CTCF | | | | | | | | | |
| | RANBP10 | | | | | | | | | |
| | ESRP2 | | | | | | | | | |
| | CYB5B | | | | | | | | | |
| | WWP2 | | | | | | | | | |
| | AARS | | | | | | | | | |
| | SF3B3 | | | | | | | | | |
| 25 | PGR | NM_000926 NM_004621 NM_001080463, | 11 | q | 100,900,355 | 117,156,404 | 13 | −1 | 2.99E−08 | 1.58 |
| | TRPC6 | NM_001377 NM_152434 NM_000019 | | | | | | | | (1.34, 1.85) |
| | DYNC2H1 | NM_004109 NM_001077691, NM_024740, | | | | | | | | |
| | CWF19L2 | NM_001077690, NM_001077692 NM_138378 | | | | | | | | |
| | ACAT1 | NM_001037954, NM_033425 NM_001082619, | | | | | | | | |
| | FDX1 | NM_138789 NM_017868 NM_032725, | | | | | | | | |
| | ALG9 | NM_001159736 NM_207343, NM_001077239 | | | | | | | | |
| | FDXACB1 | | | | | | | | | |
| | DIXDC1 | | | | | | | | | |
| | PIH1D2 | | | | | | | | | |
| | TTC12 | | | | | | | | | |
| | BUD13 | | | | | | | | | |
| | RNF214 | | | | | | | | | |
| 26 | TTC30A | NM_152275 NM_001077197, NM_016953, | 2 | q | 178,479,026 | 207,630,050 | 10 | −1 | 3.09E−08 | 1.58 |
| | PDE11A | NM_001077196, NM_001077358 NM_032168 | | | | | | | | (1.34, 1.85) |
| | WDR75 | NM_014585 NM_018897 NR_027855, | | | | | | | | |
| | SLC40A1 | NR_027856, NM_004071, NM_001162407 | | | | | | | | |
| | DNAH7 | NM_138468, NM_178231 NM_205863, | | | | | | | | |
| | CLK1 | NM_057177, NM_152526 NM_020923 | | | | | | | | |
| | ICA1L | NM_001039845 | | | | | | | | |
| | PARD3B | | | | | | | | | |
| | ZDBF2 | | | | | | | | | |
| | MDH1B | | | | | | | | | |
| 27 | PHF23 | NM_024297 NM_003985 NM_001128833, | 17 | p | 7,138,347 | 10,614,875 | 9 | −1 | 7.87E−09 | 1.57 |
| | TNK1 | NM_020899 NR_037146, NM_003809 | | | | | | | | (1.35, 1.84) |
| | ZBTB4 | NM_144607 NR_024349 NM_025099 | | | | | | | | |
| | TNFSF12 | NR_033656, NM_004853 NM_020233 | | | | | | | | |
| | CYB5D1 | | | | | | | | | |
| | LOC284023 | | | | | | | | | |
| | C17orf68 | | | | | | | | | |
| | STX8 | | | | | | | | | |
| | C17orf48 | | | | | | | | | |
| 28 | AP1G1 | NM_001128, NM_001030007 NM_002811 | 16 | q | 71,762,905 | 83,846,594 | 8 | 1 | 4.49E−08 | 1.57 |
| | PSMD7 | NM_001011880 NM_018124 NM_001100625, | | | | | | | | (1.33, 1.84) |
| | CLEC18B | NM_018455, NM_001100624 NM_022041 | | | | | | | | |
| | RFWD3 | NM_005792 NM_001537 | | | | | | | | |
| | CENPN | | | | | | | | | |
| | GAN | | | | | | | | | |
| | MPHOSPH6 | | | | | | | | | |
| | HSBP1 | | | | | | | | | |
| 29 | LOC284440 | NR_026956 NM_001077349, NM_033196 | 19 | p | 19,867,181 | 22,018,970 | 7 | −1 | 5.02E−08 | 1.57 |
| | ZNF682 | NM_052852 NM_145297, NM_001076675 | | | | | | | | (1.33, 1.84) |
| | ZNF486 | NR_027130 NM_001001415 NM 003423 | | | | | | | | |
| | ZNF626 | | | | | | | | | |
| | ZNF738 | | | | | | | | | |
| | ZNF429 | | | | | | | | | |
| | ZNF43 | | | | | | | | | |
| 30 | SI ECT2 | NM_001041 NM_018098 NM_001025205, | 3 | q | 164,696,686 | 186,505,538 | 10 | 1 | 1.49E−08 | 1.56 |
| | AP2M1 | NM_004068 NM_005787, NR_024534, | | | | | | | | (1.34, 1.82) |
| | ALG3 | NR_024533, NM_001006941 NM_002808 | | | | | | | | |
| | PSMD2 | NM_198241, NM_001194947, NM_198244, | | | | | | | | |
| | EIF4G1 | NM_198242, NM_001194946, NM_182917, | | | | | | | | |
| | IGF2BP2 | NM_004953 NM_001007225, NM_006548 | | | | | | | | |
| | C3orf65 | NR_027317 NM_016306 NR_002588 | | | | | | | | |
| | DNAJB11 | | | | | | | | | |
| | SNORA4 | | | | | | | | | |
| 31 | ZBTB1 | NM_001123329, NM_014950 NM_182526 | 14 | q | 64,971,292 | 90,798,279 | 10 | −1 | 5.61E−08 | 1.56 |
| | TMEM229B | NM_015346 NM_001168368, NM_020692 | | | | | | | | (1.33, 1.84) |
| | ZFYVE26 | NM_006925, NM_001039465 NR_027046 | | | | | | | | |
| | GALNTL1 | NM_025057 NM_001010860 NM_033104 | | | | | | | | |
| | SRSF5 | NM_199043, NM_017970 | | | | | | | | |
| | LOC145474 | | | | | | | | | |
| | C14orf45 | | | | | | | | | |
| | SAMD15 | | | | | | | | | |
| | STON2 | | | | | | | | | |
| | C14orf102 | | | | | | | | | |
| 32 | KIAA1609 | NM_020947 NM_006067, NM_001142288 | 16 | q | 84,509,966 | 90,085,937 | 11 | 1 | 6.21E−08 | 1.55 |
| | COX4NB | NR_027490, NR_027489, NM_022764, | | | | | | | | (1.32, 1.82) |
| | MTHFSD | NM_001159379, NM_001159378, NM_001159377, | | | | | | | | |
| | KLHDC4 | NM_001159380 NM_001184856, NM_001184854, | | | | | | | | |
| | SLC7A5 | NM_017566 NM_003486 NM_001739 | | | | | | | | |
| | CA5A | NM_002461 NM_000512 NM_002768, | | | | | | | | |
| | MVD | NM_001083314 NM_207514, NM_017702 | | | | | | | | |
| | GALNS | NM_001042610, NM_024043 | | | | | | | | |
| | CHMP1A | | | | | | | | | |
| | DEF8 | | | | | | | | | |
| | DBNDD1 | | | | | | | | | |
| 33 | TRPM1 | NM_002420 NR_024264 NM_001211 | 15 | q | 31,293,551 | 49,338,760 | 9 | 1 | 8.69E−08 | 1.55 |
| | LOC145845 | NM_001142762, NM_033286, NM_001142761 | | | | | | | | (1.32, 1.82) |
| | BUB1B | NM_170589, NM_144508 NM_133639 | | | | | | | | |
| | C15orf23 | NM_007236 NM_001129897, NM_018454, | | | | | | | | |
| | CASC5 | NM_016359 NM_014701, NM_001193489 | | | | | | | | |
| | RHOV | | | | | | | | | |
| | CHP | | | | | | | | | |
| | NUSAP1 | | | | | | | | | |
| | SECISBP2L | | | | | | | | | |
| 34 | INPP5K | NM_001135642, NM_130766, NM_016532 | 17 | p | 1,397,871 | 6,920,839 | 22 | −1 | 1.18E−07 | 1.54 |
| | C17orf91 | NR_028505, NR_028504, NR_028502, | | | | | | | | (1.31, 1.80) |
| | WDR81 | NR_028503 NM_001163673, NM_152348, | | | | | | | | |
| | SERPINF2 | NM_001163811, NM_001163809 NM_001165920, | | | | | | | | |
| | SMG6 | NM_000934, NM_001165921 NM_017575, | | | | | | | | |
| | SRR | NM_001170957 NM_021947 NM_014853, | | | | | | | | |
| | SGSM2 | NM_001098509 NM_024086 NM_013276 | | | | | | | | |
| | METT10D | NM_001114118, NM_018553 NM_015113 | | | | | | | | |
| | SHPK | NR_023346, NR_023347, NM_144611 | | | | | | | | |
| | C17orf85 | NM_015716, NM_153827, NM_170663, | | | | | | | | |
| | ZZEF1 | NM_001024937 NM_000080 NM_004890 | | | | | | | | |
| | CYB5D2 | NM_001171166, NM_001171168, NM_001171167, | | | | | | | | |
| | MINK1 | NM_015099 NM_001167987, NM_001167986, | | | | | | | | |
| | CHRNE | NM_001167985, NM_213726 NM_153018 | | | | | | | | |
| | SPAG7 | NM_032530 NM_001083585, NM_004703 | | | | | | | | |
| | CAMTA2 | NM_014804 NM_001142798, NM_174893, | | | | | | | | |
| | INCA1 | NM_001142799 | | | | | | | | |
| | ZFP3 | | | | | | | | | |
| | ZNF594 | | | | | | | | | |
| | RABEP1 | | | | | | | | | |
| | KIAA0753 | | | | | | | | | |
| | C17orf49 | | | | | | | | | |
| 35 | SERPINB8 | NM_002640, NM_001031848, NM_198833 | 18 | q | 61,637,263 | 77,138,282 | 6 | −1 | 1.94E−07 | 1.53 |
| | FBXO15 | NM_152676, NM_001142958 NR_024484 | | | | | | | | (1.31, 1.80) |
| | LOC400657 | NM_014643 NR_015417 NM_198531 | | | | | | | | |
| | ZNF516 | | | | | | | | | |
| | LOC284276 | | | | | | | | | |
| | ATP9B | | | | | | | | | |
| 36 | PABPC1L2B | NM_001042506 NM_183353, NM_016120 | 23 | q | 72,223,352 | 91,878,228 | 6 | 1 | 3.45E−09 | 1.53 |
| | RLIM | NM_032121 NM_000291 NM_000307 | | | | | | | | (1.33, 1.77) |
| | MAGT1 | NM_032968, NM_001168360, NM_032969, | | | | | | | | |
| | PGK1 | NM_032967, NM_001168363, NM_001168362, | | | | | | | | |
| | POU3F4 | NM_001168361, NM_014522 | | | | | | | | |
| | PCDH11X | | | | | | | | | |
| 37 | SMG7 | NM_173156, NM_201569, NM_201568, | 1 | q | 183,441,506 | 247,655,711 | 28 | 1 | 2.64E−07 | 1.52 |
| | ARPC5 | NM_001174061 NM_005717 NM_016066, | | | | | | | | (1.30, 1.79) |
| | GLRX2 | NM_197962 NM_018136 NM_020204, | | | | | | | | |
| | ASPM | NM_001014434 NM_014875 NM_005558 | | | | | | | | |
| | LHX9 | NM_001001552, NM_001199051, NM_001199052, | | | | | | | | |
| | KIF14 | NR_037583, NM_001199050 NM_002497 | | | | | | | | |
| | LAD1 | NM_016343 NM_018713 NM_004446 | | | | | | | | |
| | LEMD1 | NM_152610 NM_003676 NM_025160, | | | | | | | | |
| | NEK2 | NM_001115113 NM_022735 NM_001024227, | | | | | | | | |
| | CENPF | NM_001658, NM_001024228, NM_001024226 | | | | | | | | |
| | SLC30A10 | NM_000858, NM_001159391, NM_001159390 | | | | | | | | |
| | EPRS | NM_032800, NM_001136495, NM_001136494 | | | | | | | | |
| | C1orf65 | NM_152379 NM_022051 NR_028399, | | | | | | | | |
| | DEGS1 | NR_028400, NR_028398, NR_028397, | | | | | | | | |
| | WDR26 | NR_028393, NR_028396, NR_028395, | | | | | | | | |
| | ACBD3 | NR_028394 NM_003679 NM_001821 | | | | | | | | |
| | ARF1 | NM_003686, NM_006027, NM_130398 | | | | | | | | |
| | GUK1 | NM_016076 NM_020394 NM_001004698 | | | | | | | | |
| | C1orf198 | | | | | | | | | |
| | C1orf131 | | | | | | | | | |
| | EGLN1 | | | | | | | | | |
| | TSNAX- | | | | | | | | | |
| | DISC1 | | | | | | | | | |
| | KMO | | | | | | | | | |
| | CHML | | | | | | | | | |
| | EXO1 | | | | | | | | | |
| | PPPDE1 | | | | | | | | | |
| | ZNF695 | | | | | | | | | |
| | OR2W5 | | | | | | | | | |
| 38 | TMEM165 | NM_001009181, NM_003154 NM_002092, | 4 | q | 56,262,090 | 83,934,040 | 18 | 1 | 9.26E−14 | 1.52 |
| | C4orf14 | NM_001098477 NM_173827 NM_198889, | | | | | | | | (1.36, 1.70) |
| | UBA6 | NM_032217 NM_001133 NM_001009922, | | | | | | | | |
| | TMPRSS11E | NM_015436, NM_001008925 NM_203504, | | | | | | | | |
| | UGT2B10 | NM_203505, NM_012297 NM_003715 | | | | | | | | |
| | UGT2B7 | NM_018115 NM_005506 NM_001115007, | | | | | | | | |
| | UGT2A1 | NM_194282, NM_001115008 | | | | | | | | |
| | STATH | | | | | | | | | |
| | GRSF1 | | | | | | | | | |
| | COX18 | | | | | | | | | |
| | ANKRD17 | | | | | | | | | |
| | AFM | | | | | | | | | |
| | RCHY1 | | | | | | | | | |
| | G3BP2 | | | | | | | | | |
| | USO1 | | | | | | | | | |
| | SDAD1 | | | | | | | | | |
| | SCARB2 | | | | | | | | | |
| | LIN54 | | | | | | | | | |
| 39 | NSUN2 | NM_001193455, NM_017755 NM_001047 | 5 | p | 6,99,352 | 33,468,196 | 9 | 1 | 4.23E−07 | 1.52 |
| | SRD5A1 | NM_006999, NM_001171806, NM_001171805 | | | | | | | | (1.29, 1.78) |
| | PAPD7 | NM_012073 NM_005885 NM_004394 | | | | | | | | |
| | CCT5 | NR_033975 NM_022130 NM_152295 | | | | | | | | |
| | MARCH6 | | | | | | | | | |
| | DAP | | | | | | | | | |
| | LOC401177 | | | | | | | | | |
| | GOLPH3 | | | | | | | | | |
| | TARS | | | | | | | | | |
| 40 | SKA2 | NM_182620, NM_001100595 NM_018304 | 17 | q | 57,187,308 | 66,042,970 | 19 | 1 | 3.37E−10 | 1.51 |
| | PRR11 | NM_004859 NM_003161 NM_016125 | | | | | | | | (1.33, 1.72) |
| | CLTC | NM_181707 NM_018488 NM_005828 | | | | | | | | |
| | RPS6KB1 | NM_020198 NM_007372, NM_203499 | | | | | | | | |
| | RNFT1 | NM_017647 NM_002805, NM_001199163 | | | | | | | | |
| | C17orf64 | NM_001098426 NM_018469 NM_007215 | | | | | | | | |
| | TBX4 | NM_138363 NM_022739 NM_015462 | | | | | | | | |
| | DCAF7 | NM_002266 | | | | | | | | |
| | CCDC47 | | | | | | | | | |
| | DDX42 | | | | | | | | | |
| | FTSJ3 | | | | | | | | | |
| | PSMC5 | | | | | | | | | |
| | SMARCD2 | | | | | | | | | |
| | TEX2 | | | | | | | | | |
| | POLG2 | | | | | | | | | |
| | CCDC45 | | | | | | | | | |
| | SMURF2 | | | | | | | | | |
| | NOL11 | | | | | | | | | |
| | KPNA2 | | | | | | | | | |
| 41 | CIRBP | NM_001280, NR_023313, NR_023312 | 19 | p | 1,269,267 | 5,791,249 | 7 | −1 | 5.17E−7 | 1.51 |
| | PCSK4 | NM_017573 NM_152988, NM_001077238 | | | | | | | | (1.29, 1.78) |
| | SPPL2B | NM_001102651 NM_018074 NM_015015 | | | | | | | | |
| | ZNF554 | NM_020175, NM_001161619 | | | | | | | | |
| | CCDC94 | | | | | | | | | |
| | KDM4B | | | | | | | | | |
| | DUS3L | | | | | | | | | |
| 42 | PLCD3 | NM_133373 NM_003954 NM_001123066, | 17 | q | 43,189,008 | 45,915,046 | 6 | −1 | 4.59E−07 | 1.51 |
| | MAP3K14 | NM_001123067, NM_016835, NM_005910, | | | | | | | | (1.29, 1.78) |
| | MAPT | NM_016841, NM_016834 NR_024560 | | | | | | | | |
| | LOC100130148 | NM_001007532 NM_033413 | | | | | | | | |
| | STH | | | | | | | | | |
| | LRRC46 | | | | | | | | | |
| 43 | PRAME | NM_206954, NM_206953, NM_006115, | 22 | q | 22,890,123 | 25,055,114 | 6 | 1 | 7.07E−09 | 1.51 |
| | C22orf15 | NM_206956, NM_206955 NM_182520 | | | | | | | | (1.31, 1.74) |
| | GSTTP1 | NR_003081 NM_004175 NM_005265, | | | | | | | | |
| | SNRPD3 | NM_013430, NM_001032365, NM_001032364 | | | | | | | | |
| | GGT1 | NR_024593 | | | | | | | | |
| | POM121L10P | | | | | | | | | |
| 44 | LOC344595 | NR_028301, NR_028302 NM_018010 | 3 | q | 106,959,539 | 119,537,332 | 6 | −1 | 6.04E−07 | 1.51 |
| | IFT57 | NM_001164496, NM_018338 NM_020817 | | | | | | | | (1.28, 1.77) |
| | WDR52 | NM_033364 NM_022002, NM_003889, | | | | | | | | |
| | KIAA1407 | NM_033013 | | | | | | | | |
| | C3orf15 | | | | | | | | | |
| | NR1I2 | | | | | | | | | |
| 45 | F9 | NM_000133 NR_028345, NR_028344 | 23 | q | 138,612,895 | 153,775,233 | 8 | 1 | 1.49E−08 | 1.50 |
| | RP1-177G6-2 | NM_005342 NM_021048, NM_001011543 | | | | | | | | (1.30, 1.72) |
| | HMGB3 | NM_005362 NM_001395 NM_000033 | | | | | | | | |
| | MAGEA10 | NM_000402, NM_001042351 | | | | | | | | |
| | MAGEA3 | | | | | | | | | |
| | DUSP9 | | | | | | | | | |
| | ABCD1 | | | | | | | | | |
| | G6PD | | | | | | | | | |
| 46 | HEATR2 | NM_017802 NM_001171945, NM_001171946, | 7 | p | 766,338 | 57,247,863 | 35 | 1 | 8.28E−08 | 1.49 |
| | SUN1 | NM_001171944, NM_025154, NM_001130965 | | | | | | | | (1.29, 1.73) |
| | GET4 | NM_015949 NM_001080453 NM_001134340, | | | | | | | | |
| | INTS1 | NM_032302 NM_003751, NM_001037283 | | | | | | | | |
| | PSMG3 | NM_001097622 NM_001037163 NM_001100603, | | | | | | | | |
| | EIF3B | NM_006854 NM_182700, NM_198956 | | | | | | | | |
| | OCM | NM_012294 NM_207342, NM_001164460 | | | | | | | | |
| | C7orf70 | NR_033328, NM_018846, NR_033329, | | | | | | | | |
| | KDELR2 | NM_001172428, NM_001031710 NM_138446 | | | | | | | | |
| | SP8 | NM_016447 NM_018947 NM_002137, | | | | | | | | |
| | RAPGEF5 | NM_031243 NM_007276, NM_016587 | | | | | | | | |
| | MGC87042 | NM_000522 NM_001989 NM_001197026, | | | | | | | | |
| | KLHL7 | NM_032639, NM_001197027 NR_037669, | | | | | | | | |
| | C7orf30 | NM_001199815, NM_001199817, NM_001199816, | | | | | | | | |
| | MPP6 | NM_024051 NM_002047 NM_015060 | | | | | | | | |
| | CYCS | NM_018685 NM_032016 NM_005402 | | | | | | | | |
| | HNRNP | NM_001101648, NM_013389 NM_002541, | | | | | | | | |
| | A2B1 | NM_001003941, NM_001165036 NR_003697 | | | | | | | | |
| | CBX3 | NM_004749, NM_199122, NM_030900 | | | | | | | | |
| | HOXA13 | NM_198570 NM_001161834 NM_016139 | | | | | | | | |
| | EVX1 | NR_030766 | | | | | | | | |
| | PLEKHA8 | | | | | | | | | |
| | GGCT | | | | | | | | | |
| | GARS | | | | | | | | | |
| | AVL9 | | | | | | | | | |
| | ANLN | | | | | | | | | |
| | STARD3 | | | | | | | | | |
| | NL | | | | | | | | | |
| | RALA | | | | | | | | | |
| | NPC1L1 | | | | | | | | | |
| | OGDH | | | | | | | | | |
| | C7orf40 | | | | | | | | | |
| | TBRG4 | | | | | | | | | |
| | VWC2 | | | | | | | | | |
| | C7orf72 | | | | | | | | | |
| | CHCHD2 | | | | | | | | | |
| | LOC642006 | | | | | | | | | |
| 47 | MIR1208 | NR_031613 NM_001080431 NM_205545 | 8 | q | 129,162,362 | 146,017,805 | 14 | 1 | 1.09E−06 | 1.48 |
| | SLC45A4 | NM_023078 NM_003313 NR_033849 | | | | | | | | (1.26, 1.73) |
| | LYPD2 | NM_001136033, NM_014281, NM_078480 | | | | | | | | |
| | PYCRL | NM_000837, NM_001009184 NM_017570 | | | | | | | | |
| | TSTA3 | NM_003801 NM_001916 NM_012079 | | | | | | | | |
| | LOC100128338 | NM_004260 NM_000973, NM_033301 | | | | | | | | |
| | PUF60 | | | | | | | | | |
| | GRINA | | | | | | | | | |
| | OPLAH | | | | | | | | | |
| | GPAA1 | | | | | | | | | |
| | CYC1 | | | | | | | | | |
| | DGAT1 | | | | | | | | | |
| | RECQL4 | | | | | | | | | |
| | RPL8 | | | | | | | | | |
| 48 | NEK10 | NM_199347 NM_001171713, NM_012157 | 3 | p | 27,257,097 | 42,267,268 | 6 | −1 | 2.36E−06 | 1.47 |
| | FBXL2 | NM_015873 NM_001171171, NM_001171172, | | | | | | | | (1.25, 1.73) |
| | VILL | NM_001171174, NM_001337 NM_017886 | | | | | | | | |
| | CX3CR1 | NM_001042646, NM_014965 | | | | | | | | |
| | ULK4 | | | | | | | | | |
| | TRAK1 | | | | | | | | | |
| 49 | ZNF175 | NM_007147 NR_034159 NM_001161427, | 19 | q | 52,074,531 | 53,947,925 | 8 | −1 | 2.38E−06 | 1.47 |
| | FU30403 | NM_001161425, NM_001161426, NM_173530 | | | | | | | | (1.25, 1.72) |
| | ZNF610 | NM_032423 NM_001039886 NM_018260, | | | | | | | | |
| | ZNF528 | NM_001172655 NM_001102603, NM_198893, | | | | | | | | |
| | ZNF808 | NM_033288 NR_003148 | | | | | | | | |
| | ZNF701 | | | | | | | | | |
| | ZNF160 | | | | | | | | | |
| | LOC147804 | | | | | | | | | |
| 50 | ZNF219 | NM_016423, NM_001101672, NM_001102454 | 14 | q | 21,558,205 | 24,020,858 | 6 | −1 | 3.68E−06 | 1.46 |
| | METTL3 | NM_019852 NM_005407 NM_182728, | | | | | | | | (1.24, 1.72) |
| | SALL2 | NM_012244 NM_004050, NM_001199839 | | | | | | | | |
| | SLC7A8 | NM_033400 | | | | | | | | |
| | BCL2L2 | | | | | | | | | |
| | ZFHX2 | | | | | | | | | |
| 51 | STIP1 | NM_006819 NM_138689 NM_004451 | 11 | q | 63,953,587 | 71,159,477 | 11 | 1 | 3.45E−07 | 1.46 |
| | PPP1R14B | NM_005507 NM_020470 NM_153266 | | | | | | | | (1.26, 1.69) |
| | ESRRA | NM_007103, NM_001166102 NR_033791 | | | | | | | | |
| | CFL1 | NM_181843 NM_080658 NM_001360, | | | | | | | | |
| | YIF1A | NM_001163817 | | | | | | | | |
| | TMEM151A | | | | | | | | | |
| | NDUFV1 | | | | | | | | | |
| | DOC2GP | | | | | | | | | |
| | NUDT8 | | | | | | | | | |
| | ACY3 | | | | | | | | | |
| | DHCR7 | | | | | | | | | |
| 52 | RBBP6 | NM_006910, NM_032626, NM_018703 | 16 | p | 24,550,908 | 31,718,745 | 11 | −1 | 1.65E−06 | 1.46 |
| | TNRC6A | NM_014494 NM_015202 NM_024516 | | | | | | | | (1.25, 1.71) |
| | KIAA0556 | NM_001145524, NM_031477 NM_023931 | | | | | | | | |
| | C16orf53 | NM_001142778, NM_001142777, NM_025193 | | | | | | | | |
| | YPEL3 | NM_052874 NM_024706, NM_001172670, | | | | | | | | |
| | ZNF747 | NM_001172669, NM_001172668 NM_001136509 | | | | | | | | |
| | HSD3B7 | NR_024034 | | | | | | | | |
| | STX1B | | | | | | | | | |
| | ZNF668 | | | | | | | | | |
| | ZNF843 | | | | | | | | | |
| | KIAA06 | | | | | | | | | |
| | 64L3 | | | | | | | | | |
| 53 | ACAP2 | NM_012287 NM_003234, NM_001128148 | 3 | q | 194,995,465 | 197,511,317 | 6 | 1 | 3.04E−06 | 1.45 |
| | TFRC | NM_001105573 NM_152699 NM_014687, | | | | | | | | (1.24, 1.70) |
| | FBXO45 | NM_001145642 NM_032288, NR_027840, | | | | | | | | |
| | SENP5 | NM_001011537 | | | | | | | | |
| | KIAA0226 | | | | | | | | | |
| | FYTTD1 | | | | | | | | | |
| 54 | MYBL2 | NM_002466 NM_181799, NM_181800, | 20 | q | 42,295,709 | 48,767,852 | 7 | 1 | 5.93E−06 | 1.45 |
| | UBE2C | NM_181803, NM_007019, NM_181802, | | | | | | | | (1.23, 1.70) |
| | CTSA | NM_181801 NM_000308, NM_001127695, | | | | | | | | |
| | PLTP | NM_001167594 NM_006227, NM_182676 | | | | | | | | |
| | SLC35C2 | NM_173073, NM_015945, NM_173179 | | | | | | | | |
| | STAU1 | NM_004602, NM_017454, NM_001037328, | | | | | | | | |
| | TMEM189 | NM_017452, NM_017453 NM_001162505, | | | | | | | | |
| | | NR_027889, NM_199129 | | | | | | | | |
| 55 | GPX5 | NM_003996, NM_001509 NR_033379 | 6 | p | 28,493,789 | 30,128,711 | 6 | 1 | 1.28E−05 | 1.43 |
| | LOC401242 | NM_001195202 NM_030905 NM_030946 | | | | | | | | (1.22, 1.68) |
| | LOC100129636 | NM_052828, NM_006778 | | | | | | | | |
| | OR2J2 | | | | | | | | | |
| | OR14J1 | | | | | | | | | |
| | TRIM10 | | | | | | | | | |
| 56 | GTSE1 | NM_016426 NM_014577 NR_026993 | 22 | q | 46,692,638 | 50,746,002 | 8 | 1 | 3.51E−09 | 1.40 |
| | BRD1 | NM_014838 NM_024324, NM_001135101 | | | | | | | | (1.25, 1.57) |
| | LOC90834 | NM_001001852 NM_025204 NM_012401 | | | | | | | | |
| | ZBED4 | | | | | | | | | |
| | CRELD2 | | | | | | | | | |
| | PIM3 | | | | | | | | | |
| | TRABD | | | | | | | | | |
| | PLXNB2 | | | | | | | | | |
| 57 | TRAM1 | NM_014294 NM_172037 NM_001164380, | 8 | q | 71,485,453 | 97,247,862 | 11 | 1 | 6.07E−05 | 1.39 |
| | RDH10 | NM_014393, NM_001164384, NM_001164382, | | | | | | | | (1.18, 1.64) |
| | STAU2 | NM_001164381, NM_001164385, NM_001164383 | | | | | | | | |
| | ZFAND1 | NM_001170797, NM_001170796, NR_033193, | | | | | | | | |
| | E2F5 | NR_033194, NM_024699, NR_033195, | | | | | | | | |
| | CNGB3 | NR_033196 NM_001951, NM_001083588, | | | | | | | | |
| | NBN | NM_001083589 NM_019098 NM_002485 | | | | | | | | |
| | FAM92A1 | NM_145269 NM_001122827, NM_017697, | | | | | | | | |
| | ESRP1 | NM_001122825, NM_001122826, NM_001034915 | | | | | | | | |
| | INTS8 | NM_017864 NM_006294, NM_001199975 | | | | | | | | |
| | UQCRB | | | | | | | | | |
| 58 | HIST1H1C | NM_005319 NM_021064 NM_080596 | 6 | p | 26,055,968 | 27,833,576 | 6 | 1 | 5.86E−05 | 1.37 |
| | HIST1H2AG | NR_036096 NM_021066 NM_003511 | | | | | | | | (1.17, 1.59) |
| | HIST1H2AH | | | | | | | | | |
| | MIR3143 | | | | | | | | | |
| | HIST1H2AJ | | | | | | | | | |
| | HIST1H2AL | | | | | | | | | |
| 59 | AP4E1 | NM_007347 NM_000103, NM_031226 | 15 | q | 51,200,946 | 52,569,397 | 6 | 1 | 2.04E−07 | 1.31 |
| | CYP19A1 | NM_014548, NM_001142885 NM_014547 | | | | | | | | (1.18, 1.45) |
| | TMOD2 | NM_002748 NR_031670 | | | | | | | | |
| | TMOD3 | | | | | | | | | |
| | MAPK6 | | | | | | | | | |
| | MIR1266 | | | | | | | | | |

TABLE 2B
16 Enhanced Mapped Strings based on Identified Exons from the Providence Dataset
RM-Corrected Absolute Standardized Hazard Ratios for Enhanced Strings
Providence NGS Study, All Patients

| | | | RM-Corrected Absolute |
|---|---|---|---|
| Enhanced | | | Standardized Hazard Ratio |
| String No. | Genes in Enhanced String | p-Value | (95% Confidence Interval) |
| 1 | ZNF317 ZNF44 ZNF440 ZNF486 ZNF626 ZNF682 ZNF738 | 2.87E−11 | 1.72 (1.46, 2.01) |
| 2 | RAB11B ZNF317 ZNF44 ZNF440 ZNF486 ZNF626 ZNF682 ZNF738 | 1.18E−10 | 1.69 (1.44, 1.99) |
| 3 | AARS AP1G1 CA5A CENPN CRNDE CYB5B DBNDD1 DEF8 ESRP2 GALNS GAN GFOD2 | 3.50E−09 | 1.62 (1.38, 1.90) |
| | HYDIN MMP15 MTHFSD MVD NQO1 NUDT21 RFWD3 SF3B3 | | |
| 4 | ZNF317 ZNF44 ZNF440 | 1.15E−07 | 1.55 (1.32, 1.82) |
| 5 | CCDC103 LRRC37A4 MAP3K14 | 5.56E−08 | 1.55 (1.32, 1.81) |
| 6 | IPW PAR-SN SNORD115-40 SNORD116-12 | 1.19E−08 | 1.50 (1.31, 1.73) |
| 7 | RAET1L ULBP3 | 2.74E−07 | 1.49 (1.28, 1.73) |
| 8 | C7orf30 DDX56 EVX1 GARS GGCT HOXA13 MGC87042 TBRG4 | 5.26E−05 | 1.33 (1.16, 1.53) |
| 9 | AP4E1 GABPB1 LOC145845 MAPK6 MIR1266 SNORD115-40 TMOD2 TMOD3 | 1.56E−07 | 1.32 (1.19, 1.46) |
| 10 | AP4E1 ARPP19 GABPB1 MAPK6 MIR1266 MYO5A TMOD2 TMOD3 | 3.42E−10 | 1.25 (1.17, 1.34) |
| 11 | ASPN CENPP ECM2 OGN OMD | 1.39E−07 | 1.54 (1.31, 1.81) |
| 12 | CCDC45 CCDC47 CLTC DCAF7 DDX42 FTSJ3 KPNA2 NOL11 POLG2 PSMC5 RNFT1 | 9.94E−09 | 1.46 (1.29, 1.67) |
| | RPS6KB1 SMARCD2 SMURF2 TEX2 | | |
| 13^{a} | AURKA BIRC5 MKI67 MYBL2 | 9.52E−04 | 1.30 (1.11, 1.52) |
| 14^{b} | ASPM BUB1B CASC5 CENPE CENPF DLGAP5 HJURP KIF11 KIF14 MKI67 NUSAP1 | 1.51E−03 | 1.28 (1.10, 1.49) |
| | PRR11 | | |
| 15^{c} | TMEM165 UBA6 GRSF1 RCHY1 G3BP2 USO1 SDAD1 SCARB2 GGCT GARS | 2.87E−13 | 1.47 (1.32, 1.63) |
| 16^{d} | ZNF554 ZNF317 ZNF653 ZNF823 ZNF441 ZNF440 ZNF763 ZNF433 ZNF844 ZNF788 | 4.24E−12 | 1.72 (1.47, 2.00) |
| | ZNF20 ZNF625 ZNF44 ZNF563 ZNF442 ZNF564 ZNF682 ZNF486 ZNF626 ZNF738 | | |
| | ZNF429 ZNF43 ZNF540 ZNF576 ZNF227 ZNF175 ZNF610 ZNF528 ZNF808 ZNF701 | | |
| | ZNF160 ZNF211 | | |

^{a}As a standard for comparison, we treated the ODX breast proliferation genes as a string and computed the HR and p-value. CCNB1 was not significant and was omitted. As shown, the ODX proliferation string is much weaker than most of the enhanced string HR and p-values.
^{b}We also treated other highly co-expressed proliferation genes as a string (R ≧ 0.8) and computed the enhanced string HR and p-value. This enhanced string's HR and p-value was also rather unremarkable relative to other enhanced strings.
^{c}Two small strings of G3BP2 co-expressed genes (R ≧ 0.6) from chromosomes 4 and 7 were also treated as an enhanced string.
^{d}Chromosome 19 contains 3 clusters of ZNF gene strings and multiple pairs and singleton ZNF genes. These were combined to give a highly significant HR and p-value as an enhanced string.

TABLE 2C
25 Enhanced Mapped Strings based on Identified Exons from ER+
Patients in the Providence Patient Set
RM-Corrected Absolute Standardized Hazard Ratios for Enhanced Strings
Providence NGS Study, ER-Positive Patients

| | | | RM-Corrected Absolute |
|---|---|---|---|
| Enhanced | | | Standardized Hazard Ratio |
| String No. | Genes in Enhanced String | p-Value | (95% Confidence Interval) |
| 1 | ZNF317 ZNF44 ZNF440 ZNF486 ZNF626 ZNF682 ZNF738 | 9.57E−11 | 1.70 (1.45, 1.99) |
| 2 | RAB11B ZNF317 ZNF44 ZNF440 ZNF486 ZNF626 ZNF682 ZNF738 | 1.13E−10 | 1.69 (1.44, 1.98) |
| 3 | CCDC103 LRRC37A4 MAP3K14 | 7.46E−09 | 1.60 (1.37, 1.88) |
| 4^{a} | AARS AP1G1 CA5A CENPN CRNDE CYB5B DBNDD1 DEF8 ESRP2 GALNS GAN GFOD2 | 3.21E−09 | 1.60 (1.37, 1.87) |
| | HYDIN MMP15 MTHFSD MVD NQO1 NUDT21 RFWD3 SF3B3 | | |
| 5^{b} | CRNDE NUDT21 MMP15 GFOD2 ESRP2 CYB5B NQO1 AARS SF3B3 AP1G1 RFWD3 | 1.57E−08 | 1.56 (1.34, 1.82) |
| | CENPN GAN MTHFSD CA5A MVD GALNS DEF8 DBNDD1 | | |
| 6 | IPW PAR-SN SNORD115-40 SNORD116-12 | 4.05E−09 | 1.57 (1.35, 1.83) |
| 7 | RAET1L ULBP3 | 8.57E−08 | 1.54 (1.32, 1.81) |
| 8 | ZNF317 ZNF44 ZNF440 | 2.87E−07 | 1.51 (1.29, 1.77) |
| 9 | AP4E1 GABPB1 LOC145845 MAPK6 MIR1266 SNORD115-40 TMOD2 TMOD3 | 1.48E−09 | 1.40 (1.26, 1.56) |
| 10 | AP4E1 ARPP19 GABPB1 MAPK6 MIR1266 MYO5A TMOD2 TMOD3 | 3.20E−12 | 1.32 (1.22, 1.43) |
| 11^{c} | C7orf30 DDX56 EVX1 GARS GGCT HOXA13 MGC87042 TBRG4 | 1.89E−05 | 1.31 (1.16, 1.49) |
| 12 | ZNF227 ZNF317 ZNF44 ZNF440 ZNF486 ZNF626 ZNF682 ZNF701 ZNF738 ZNF829 | 6.21E−11 | 1.71 (1.46, 2.01) |
| 13 | CCNA2 COX7B2 DCK FAM184B FLJ14186 G3BP2 GRSF1 HMX1 MIR4275 MIR548I2 | 8.55E−11 | 1.57 (1.37, 1.80) |
| | NPY1R PCGF3 SCARB2 USO1 ZNF141 ZNF595 | | |
| 14 | AAGAB CRABP1 FAM169B MEX3B NTRK3 RFX7 RHCG SNORD18C | 1.16E−07 | 1.52 (1.30, 1.78) |
| 15 | KRT78 KRT83 | 1.47E−07 | 1.52 (1.30, 1.77) |
| 16 | CCNA2 DCK G3BP2 GRSF1 SCARB2 USO1 | 4.19E−07 | 1.44 (1.25, 1.65) |
| 17 | IPW PAR-SN SNORD116-12 | 2.90E−06 | 1.42 (1.23, 1.64) |
| 18 | PAR-SN SNORD116-12 | 8.63E−07 | 1.40 (1.22, 1.60) |
| 19 | CCNA2 COX7B2 DCK FLJ14186 G3BP2 GRSF1 MIR4275 SCARB2 USO1 | 8.74E−06 | 1.38 (1.20, 1.60) |
| 20 | GPAA1 PYCRL | 1.96E−04 | 1.36 (1.16, 1.60) |
| 21 | GPAA1 PRPF38A PYCRL SYNPO | 0.000279 | 1.35 (1.15, 1.58) |
| 22 | AAGAB AP4E1 ARPP19 CRABP1 FAM169B GABPB1 IPW LOC145845 MAPK6 MEX3B | 1.39E−06 | 1.35 (1.19, 1.52) |
| | MIR1266 MYO5A NTRK3 PAR-SN RFX7 RHCG SNORD115-40 SNORD116-12 SNORD18C | | |
| | TMOD2 TMOD3 | | |
| 23 | COX7B2 DCK G3BP2 GRSF1 MIR4275 SCARB2 USO1 | 9.7E−06 | 1.34 (1.18, 1.53) |
| 24 | IPW PAR-SN | 0.001872 | 1.29 (1.10, 1.51) |
| 25 | AAGAB AP4E1 ARPP19 FAM169B GABPB1 MAPK6 MYO5A TMOD2 TMOD3 | 1.32E−09 | 1.24 (1.16, 1.33) |

^{a}The chromosome 16 q arm contains two large strings that are separated by the gene hydin. Despite having an opposite direction of association with the two strings, including the gene hydin, increases the prognostic strength of the enhanced string (see note b below for comparison).
^{b}This enhanced string is identical to the one above except that it does not contain hydin.
^{c}This string contains the entire set of genes that are prognostic on the chromosome 7 p arm in ER positive patients.

TABLE 5A
79 Mapped Strings based on Identified Introns from the Providence Dataset

| | Strings Based on Identified Introns | | |
|---|---|---|---|
| | | Chromosome | |
| String No. | Genes in Run | (23 = X, 24 = Y) | Arm |
| 1 | EFNA5 FAM170A LOX ALDH7A1 PRRC1 SEC24A TRPC7 | 5 | q |
| | CDC25C HSPA9 NRG2 PCDHGA7 PCDHGA6 PCDHGB4 | | |
| | DIAPH1 | | |
| 2 | SLC34A1 PRR7 AACSP1 CANX MAML1 SQSTM1 | 5 | q |
| | RASGEF1C GNB2L1 | | |
| 3 | RAD52 DCP1B PARP11 C12orf5 VAMP1 ACRBP ZNF384 | 12 | p |
| | C12orf53 LAG3 LRRC23 ENO2 ATN1 C12orf57 FOXJ2 | | |
| | NECAP1 CLEC4A FAM66C | | |
| 4 | ASTN2 TRIM32 MEGF9 LOC100288842 C5 RAB14 | 9 | q |
| | ZBTB26 ZBTB34 RALGPS1 SLC2A8 ZNF79 C9orf117 | | |
| 5 | HMGCR OTP HOMER1 ARRDC3 LIX1 SLCO6A1 | 5 | q |
| 6 | DIO1 LRRC42 C1orf168 DAB1 OMA1 WDR78 SLC35D1 | 1 | p |
| | HHLA3 PTGER3 NEGR1 C1orf173 CRYZ TYW3 FAM73A | | |
| | RPF1 TGFBR3 C1orf146 BCAR3 LOC100129046 ALG14 | | |
| | CCDC76 SLC25A24 TMEM167B KIAA1324 SARS MYBPHL | | |
| | GSTM4 GSTM2 | | |
| 7 | RPGRIP1 REM2 SLC7A8 HOMEZ ZFHX2 DHRS4 GMPR2 | 14 | q |
| | ADCY4 RIPK3 CMA1 | | |
| 8 | NOSTRIN LRP2 BBS5 C2orf77 KLHL23 CYBRD1 DYNC1I2 | 2 | q |
| | SLC25A12 PDE11A RBM45 WDR75 SLC40A1 ANKAR | | |
| | DNAH7 | | |
| 9 | PHLDB1 HINFP PDZD3 CCDC153 GRIK4 TBCEL TECTA | 11 | q |
| | VWA5A SPA17 KCNJ5 ADAMTS8 | | |
| 10 | ISOC2 NLRP8 LOC386758 ZFP28 ZNF71 ZNF835 ZNF132 | 19 | q |
| | CHMP2A | | |
| 11 | POU4F3 TCERG1 SPINK5 GLRA1 CLINT1 RNF145 UBLCP1 | 5 | q |
| | LOC285629 GABRG2 HMMR TLX3 NPM1 | | |
| 12 | LMAN1L CPLX3 COX5A FBXO22 SCAPER LINGO1 | 15 | q |
| | CRABP1 RASGRF1 KIAA1024 | | |
| 13 | SECISBP2 NOL8 CENPP BICD2 ANKRD19 ZNF484 FGD3 | 9 | q |
| | SUSD3 C9orf89 NINJ1 FAM120A PHF2 FBP1 C9orf130 | | |
| | C9orf102 SLC35D2 ZNF510 ZNF782 LOC57653 TSTD2 | | |
| | NCBP1 XPA C9orf156 | | |
| 14 | XPO1 RAB1A MEIS1 PPP3R1 GKN1 GFPT1 C2orf78 | 2 | p |
| 15 | CIZ1 SPTAN1 SET ENDOG LRRC8A PHYHD1 NUP188 | 9 | q |
| | SH3GLB2 TOR1A USP20 GPR107 ABL1 TTF1 AK8 C9orf9 | | |
| | TSC1 SURF1 SURF2 ADAMTSL2 | | |
| 16 | ARPP21 VILL DLEC1 CX3CR1 ZNF619 TRAK1 | 3 | p |
| 17 | LPPR2 EPOR ZNF627 ZNF491 ZNF763 ZNF433 ZNF788 | 19 | p |
| | ZNF625 ZNF136 ZNF44 ZNF563 ZNF799 ZNF709 ZNF564 | | |
| | ZNF490 ZNF791 HOOK2 LYL1 | | |
| 18 | ERRFI1 ENO1 KIF1B VPS13D DHRS3 PRAMEF11 | 1 | p |
| 19 | PYY NAGS TMEM101 TMUB2 UBTF PLCD3 ACBD4 | 17 | q |
| | MAP3K14 LOC100128977 MAPT NFE2L1 | | |
| 20 | LRP11 ULBP1 ULBP3 PPP1R14C PLEKHG1 FBXO5 EZR | 6 | q |
| | LOC100129518 IGF2R MAP3K4 TTLL2 C6orf124 | | |
| 21 | POLQ PDIA5 PTPLB ALG1L PODXL2 GP9 | 3 | q |
| 22 | CIC TEX101 CADM4 ZNF223 ZNF284 LOC100379224 | 19 | q |
| | ZFP112 CLPTM1 CLASRP TRAPPC6A SIX5 DMWD MEIS3 | | |
| | EMP3 CYTH2 | | |
| 23 | SARNP APOF MIP NXPH4 INHBE LRIG3 | 12 | q |
| 24 | ZNF175 ZNF616 ZNF836 ZNF610 ZNF528 ZNF578 | 19 | q |
| | ZNF137P | | |
| 25 | C17orf102 LYZL6 LHX1 NEUROD2 ZPBP2 KRT27 KRT16 | 17 | q |
| 26 | TMEM132A PGA3 FADS2 INCENP SLC3A2 SLC22A6 | 11 | q |
| 27 | RRP8 DCHS1 ZNF214 SYT9 TRIM66 NRIP3 SCUBE2 | 11 | p |
| 28 | C17orf97 RNMTL1 ABR YWHAE MYO1C INPP5K SCARF1 | 17 | p |
| | PRPF8 SERPINF1 RTN4RL1 SMG6 SGSM2 TRPV1 | | |
| | C17orf85 ZZEF1 CYB5D2 PLD2 MINK1 ENO3 CAMTA2 | | |
| | INCA1 KIF1C ZFP3 ZNF594 LOC100130950 RABEP1 | | |
| | KIAA0753 ALOX12 C17orf49 CLEC10A GPS2 NEURL4 | | |
| | NLGN2 TNFSF12_TNFSF13 CYB5D1 C17orf68 WDR16 | | |
| | MYH2 MYH3 TEKT3 PIGL CENPV LRRC48 ALKBH5 FLII | | |
| 29 | FLJ31306 TOMM20L JKAMP C14orf38 RTN1 C14orf135 | 14 | q |
| 30 | SCFD2 TMEM165 SRP72 UBA6 TMPRSS11E UGT2B10 | 4 | q |
| | UGT2B7 UGT2A1 AMTN AMBN ANKRD17 ALB AFM | | |
| | EPGN RCHY1 G3BP2 USO1 NUP54 SCARB2 CXCL13 | | |
| | RASGEF1B SEC31A IBSP | | |
| 31 | VEGFA PLA2G7 TNFRSF21 DEFB112 IL17F GSTA5 PRIM2 | 6 | p |
| 32 | FGF12 ACAP2 TFRC ZDHHC19 RNF168 SENP5 DLG1 | 3 | q |
| | KIAA0226 LRCH3 IQCG | | |
| 33 | ZNF415 ZNF347 ZNF665 ZNF813 PRPF31 LILRA2 | 19 | q |
| 34 | NIPBL NUP155 WDR70 RPL37 HMGCS1 PAIP1 | 5 | p |
| 35 | DPM3 KRTCAP2 MSTO1 IQGAP3 ITLN2 PVRL4 NUF2 | 1 | q |
| | MPZL1 TBX19 C1orf9 PRDX6 | | |
| 36 | MCF2 RP1_177G6_2 SPANXN2 SLITRK2 MAGEA5 | 23 | q |
| | MAGEA10 MAGEA12 | | |
| 37 | RNF125 ZSCAN30 ZNF396 SETBP1 SLC14A2 HAUS1 | 18 | q |
| 38 | CNTNAP1 LOC100190938 RAMP2 VAT1 TMEM106A | 17 | q |
| | LOC100130581 | | |
| 39 | MYOG CHI3L1 HSD11B1 NEK2 CENPF EPRS IARS2 | 1 | q |
| | RAB3GAP2 AIDA SRP9 ACBD3 LIN9 C1orf198 TTC13 | | |
| | C1orf124 EGLN1 SIPA1L2 IRF2BP2 RBM34 B3GALNT2 | | |
| | CHRM3 KMO OPN3 EXO1 PPPDE1 AHCTF1 ZNF695 | | |
| 40 | FANCI RHCG LOC283761 C15orf42 ZNF710 IDH2 | 15 | q |
| | SEMA4B NGRN BLM PRC1 LRRC28 | | |
| 41 | CNGB3 RIPK2 LRRC69 RAD54B ESRP1 UQCRB PTDSS1 | 8 | q |
| | STK3 VPS13B RNF19A PABPC1 YWHAZ FLJ42969 GRHL2 | | |
| | NCALD AZIN1 SLC25A32 LRP12 OXR1 EIF3E EIF3H EXT1 | | |
| | TAF2 MTBP WDR67 FAM83A NDRG1 PTK2 SLURP1 | | |
| | LYPD2 LY6D LY6H C8orf73 SPATC1 HEATR7A ADCK5 | | |
| | CYHR1 FOXH1 | | |
| 42 | CREBL2 DDX47 HEBP1 RERG DERA KIAA0528 CASC1 | 12 | p |
| | AMN1 C12orf35 | | |
| 43 | SMPDL3A CENPW C6orf58 PTPRK MED23 VNN3 | 6 | q |
| | LOC285735 MAP7 PERP HEBP2 NHSL1 REPS1 GPR126 | | |
| | HIVEP2 PHACTR2 | | |
| 44 | IVNS1ABP FAM5C UCHL5 CDC73 CFHR1 CFHR5 ASPM | 1 | q |
| | KIF14 C1orf106 | | |
| 45 | CPLX2 FAF2 CDHR2 GPRIN1 UIMC1 ZNF346 NSD1 | 5 | q |
| 46 | MICAL3 UFD1L CDC45 PRAME LOC91316 CHCHD10 | 22 | q |
| 47 | PRR11 DHX40 CLTC PTRH2 TMEM49 HEATR6 C17orf64 | 17 | q |
| | BCAS3 TBX4 BRIP1 MED13 STRADA CCDC47 DDX42 | | |
| | FTSJ3 SMARCD2 TCAM1P CSHL1 TEX2 POLG2 DDX5 | | |
| | CCDC45 SMURF2 LOC146880 PLEKHM1P NOL11 BPTF | | |
| | KPNA2 FAM20A | | |
| 48 | SHCBP1 ORC6 ITFG1 PHKB HEATR3 BRD7 NUDT21 MT4 | 16 | q |
| | MT1G NUP93 HERPUD1 COQ9 GPR56 MMP15 CNOT1 | | |
| | CDH8 DYNC1LI2 CBFB EXOC3L GFOD2 NUTF2 DUS2L | | |
| | ESRP2 WWP2 PDXDC2P AARS DDX19A COG4 SF3B3 | | |
| | MTSS1L | | |
| 49 | KIF4A PGK1 TAF9B PCDH11X DIAPH2 CENPI | 23 | q |
| 50 | KIAA1609 USP10 KIAA0182 COX4I1 SLC7A5 BANP | 16 | q |
| | ZC3H18 IL17C MVD PABPN1L ANKRD11 LOC100128881 | | |
| | FANCA LOC100130015 | | |
| 51 | SRD5A1 PAPD7 MTRR MARCH6 DAP DROSHA C5orf22 | 5 | p |
| | GOLPH3 ZFR TARS | | |
| 52 | PPP1CB GALNT14 MEMO1 BIRC6 FAM98A ATL2 EML4 | 2 | p |
| | LRPPRC PPM1B C2orf34 SIX2 SRBD1 PSME4 | | |
| 53 | PINK1 NBPF3 HSPG2 TCEA3 HMGCL MYOM3 PAQR7 | 1 | p |
| | UBXN11 | | |
| 54 | C14orf50 TMEM229B EXD2 GALNTL1 DNAL1 EIF2B2 | 14 | q |
| | KIAA1737 POMT2 | | |
| 55 | MOV10 PHTF1 SYT6 ATP1A1OS TRIM45 SPAG17 WARS2 | 1 | p |
| 56 | HEATR2 SUN1 INTS1 PSMG3 MAD1L1 EIF3B RNF216L | 7 | p |
| | EIF2AK1 KDELR2 RAPGEF5 MGC87042 TOMM7 | | |
| | FAM126A C7orf30 NPY MPP6 DFNA5 HNRNPA2B1 CBX3 | | |
| | HOXA11 EVX1 HIBADH TAX1BP1 PLEKHA8 ZNRF2 NOD1 | | |
| | GGCT GARS SEPT7 ANLN VPS41 CDK13 PPIA TBRG4 | | |
| | IGFBP3 ZNF716 | | |
| 57 | PHIP TTK ME1 RIPPLY2 PRDM1 ATG5 PDSS2 | 6 | q |
| 58 | TMEM221 COMP ZNF626 ZNF85 ZNF738 ZNF429 | 19 | p |
| 59 | FAM115A ZNF425 ZNF862 LOC401431 PRKAG2 | 7 | q |
| | LOC100132707 | | |
| 60 | C15orf23 CASC5 RAD51 RHOV INO80 OIP5 NUSAP1 | 15 | q |
| | UBR1 ELL3 | | |
| 61 | ACER2 IFT74 TOPORS NDUFB6 DNAJA1 DCTN3 RUSC2 | 9 | p |
| | CREB3 TMEM8B | | |
| 62 | MRPL54 PIAS4 MAP2K2 SIRT6 ANKRD24 HDGFRP2 | 19 | p |
| | KDM4B ACSBG2 MLLT1 GTF2F1 TRIP10 ZNF358 XAB2 | | |
| | MAP2K7 | | |
| 63 | MGC3771 ZNF263 TIGD7 ZNF434 ZNF597 NAT15 | 16 | p |
| | CREBBP C16orf5 MGRN1 C16orf71 LOC440335 NAGPA | | |
| | ABAT NUBP1 CLEC16A MKL2 NOMO2 ACSM5 LYRM1 | | |
| | C16orf52 EEF2K RBBP6 NSMCE1 FLJ21408 CCDC101 | | |
| | SH2B1 | | |
| 64 | MVP LOC595101 CD2BP2 TBC1D10B SEPT1 ZNF768 | 16 | p |
| | PHKG2 BCL7C STX1B C16orf58 | | |
| 65 | ZNF721 PIGG PDE6B PCGF3 FGFRL1 TNIP2 C4orf44 | 4 | p |
| | RGS12 LRPAP1 STK32B CRMP1 WFS1 MAN2B2 | | |
| | LOC100129931 GRPEL1 | | |
| 66 | BAI2 CCDC28B IQCC MTMR9LP ADC TEKT2 MAP7D1 | 1 | p |
| | OSCP1 DNALI1 RSPO1 | | |
| 67 | STON2 GALC TTC8 KCNK13 GPR68 ATXN3 | 14 | q |
| 68 | FCGBP CCDC97 B9D2 TMEM91 BCKDHA B3GNT8 | 19 | q |
| 69 | IFRD1 C7orf53 FAM3C LOC154860 SPAM1 SND1 CALU | 7 | q |
| 70 | ZNF189 FSD1L IKBKAP PTGR1 C9orf80 C9orf43 | 9 | q |
| 71 | CRNN LCE3E LCE1C SPRR1B S100A8 ILF2 | 1 | q |
| 72 | NAA15 SLC10A7 FBXW7 FGB FGG ACCN5 TDO2 | 4 | q |
| 73 | LDLRAD3 COMMD9 C11orf74 LRRC4C ALKBH3 AGBL2 | 11 | p |
| 74 | C10orf18 USP6NL MCM10 ABI1 LOC729668 PARD3 | 10 | p |
| 75 | C19orf20 RNF126 FSTL3 PALM MED16 STK11 | 19 | p |
| 76 | GTSE1 BRD1 ZBED4 TRABD SELO PPP6R2 ADM2 | 22 | q |
| | RPL23AP82 | | |
| 77 | DIXDC1 PIH1D2 C11orf34 TTC12 ANKK1 USP28 RNF214 | 11 | q |
| 78 | SPATA5L1 CTXN2 SLC12A1 CEP152 SECISBP2L GALK2 | 15 | q |
| | GABPB1 USP8 TRPM7 SPPL2A AP4E1 TNFAIP8L3 | | |
| | CYP19A1 DMXL2 TMOD2 TMOD3 MAPK6 ARPP19 | | |
| 79 | AP1G1 KIAA0174 PSMD7 GLG1 RFWD3 CFDP1 | 16 | q |

| | Strings Based on Identified Introns | | | | | |
|---|---|---|---|---|---|---|
| | | | | | | RM- |
| | | | | | | Corrected |
| | Exemplary | Exemplary | | | | Absolute |
| | Start | End | | | | Standardized |
| | Location of | Location of | # | | | Hazard Ratio |
| | First Gene in | Last Gene in | Genes | Direction of | | (95% Conf. |
| String No. | Run | Run | in Run | Association | p-value | Int.) |
| 1 | 106,712,589 | 140,998,622 | 14 | 1 | 1.89E−15 | 1.88 (1.61, |
| | | | | | | 2.20) |
| 2 | | 180,670,906 | 8 | 1 | 1.46E−12 | 1.83 (1.55, |
| | 176,811,431 | | | | | 2.17) |
| 3 | | 8,353,596 | 17 | −1 | 7.02E−12 | 1.82 (1.53, |
| | 1,021,254 | | | | | 2.16) |
| 4 | | 130,476,303 | 12 | −1 | 1.03E−12 | 1.80 (1.53, |
| | | | | | | 2.11) |
| 5 | 119,187,503 | 101,834,720 | 6 | 1 | 9.77E−12 | 1.77 (1.50, |
| | | | | | | 2.09) |
| 6 | 74,632,992 | 110,226,619 | 28 | −1 | 1.47E−10 | 1.75 (1.48, |
| | 54,359,860 | | | | | 2.08) |
| 7 | | 24,977,471 | 10 | −1 | 1.43E−10 | 1.74 (1.47, |
| | | | | | | 2.05) |
| 8 | | 196,933,536 | 14 | −1 | 6.18E−10 | 1.70 (1.44, |
| | | | | | | 2.01) |
| 9 | 21,819,375 | 130,298,539 | 11 | −1 | 2.09E−10 | 1.69 (1.44, |
| | | | | | | 1.99) |
| 10 | 169,643,048 | 59,066,486 | 8 | −1 | 1.94E−09 | 1.68 (1.42, |
| | | | | | | 1.99) |
| 11 | | 170,837,888 | 12 | 1 | 3.07E−09 | 1.68 (1.41, |
| | 118,477,212 | | | | | 1.99) |
| 12 | | 79,764,642 | 9 | 1 | 4.43E−10 | 1.68 (1.43, |
| | 55,964,345 | | | | | 1.97) |
| 13 | | 100,684,852 | 23 | −1 | 1.26E−09 | 1.67 (1.41, |
| | 145,718,586 | | | | | 1.97) |
| 14 | | 74,044,274 | 7 | 1 | 1.28E−09 | 1.66 (1.41, |
| | 75,105,193 | | | | | 1.96) |
| 15 | | 136,440,641 | 19 | −1 | 3.41E−09 | 1.66 (1.40, |
| | 91,933,411 | | | | | 1.96) |
| 16 | | 42,267,382 | 6 | −1 | 9.05E−09 | 1.65 (1.39, |
| | | | | | | 1.95) |
| 17 | | 13,213,974 | 18 | −1 | 6.35E−09 | 1.64 (1.39, |
| | 61,705,068 | | | | | 1.94) |
| 18 | 130,928,343 | 12,891,264 | 6 | 1 | 6.94E−10 | 1.64 (1.40, |
| | | | | | | 1.92) |
| 19 | | 46,138,907 | 11 | −1 | 4.26E−09 | 1.64 (1.39, |
| | 35,680,665 | | | | | 1.93) |
| 20 | 11,466,061 | 168,227,476 | 12 | 1 | 2.03E−08 | 1.64 (1.38, |
| | | | | | | 1.94) |
| 21 | | 128,781,253 | 6 | 1 | 1.60E−09 | 1.64 (1.39, |
| | 8,071,778 | | | | | 1.92) |
| 22 | 42,030,106 | 48,985,571 | 15 | −1 | 9.64E−09 | 1.62 (1.37, |
| | | | | | | 1.90) |
| 23 | 150,139,893 | 59,314,319 | 6 | 1 | 3.88E−08 | 1.61 (1.36, |
| | | | | | | 1.91) |
| 24 | 121,150,272 | 53,103,405 | 7 | −1 | 4.65E−08 | 1.60 (1.35, |
| | 42,788,816 | | | | | 1.89) |
| 25 | | 39,769,079 | 7 | 1 | 3.29E−08 | 1.60 (1.35, |
| | | | | | | 1.89) |
| 26 | 56,146,246 | 62,752,495 | 6 | 1 | 3.20E−08 | 1.60 (1.35, |
| | 52,074,530 | | | | | 1.88) |
| 27 | | 9,113,150 | 7 | −1 | 8.60E−08 | 1.60 (1.34, |
| | 32,901,141 | | | | | 1.89) |
| 28 | 60,691,912 | 18,162,230 | 45 | −1 | 1.23E−07 | 1.59 (1.34 |
| | 6,621,143 | | | | | 1.89) |
| 29 | 260,117 | 60,601,532 | 6 | −1 | 1.45E−07 | 1.59 (1.34, |
| | | | | | | 1.88) |
| 30 | | 88,733,601 | 23 | 1 | 6.02E−12 | 1.58 (1.39, |
| | | | | | | 1.80) |
| 31 | | 57,513,376 | 7 | 1 | 3.09E−08 | 1.57 (1.34, |
| | | | | | | 1.84) |
| 32 | | 197,686,886 | 10 | 1 | 1.12E−08 | 1.57 (1.34, |
| | 58,732,082 | | | | | 1.83) |
| 33 | 53,739,150 | 55,099,027 | 6 | −1 | 1.93E−07 | 1.57 (1.32, |
| | | | | | | 1.86) |
| 34 | | 43,557,521 | 6 | 1 | 4.22E−08 | 1.57 (1.34, |
| | | | | | | 1.84) |
| 35 | 43,737,945 | 173,457,946 | 11 | 1 | 2.71E−07 | 1.56 (1.32, |
| | 191,857,181 | | | | | 1.85) |
| 36 | | 151,903,184 | 7 | 1 | 1.51E−07 | 1.56 (1.32, |
| | 53,611,131 | | | | | 1.84) |
| 37 | 36,876,860 | 43,708,299 | 6 | −1 | 3.59E−07 | 1.56 (1.31, |
| | 155,112,366 | | | | | 1.85) |
| 38 | | 41,466,266 | 6 | −1 | 4.10E−09 | 1.56 (1.34, |
| | 138,663,929 | | | | | 1.81) |
| 39 | | 247,171,395 | 27 | 1 | 5.26E−07 | 1.55 (1.31, |
| | 29,598,444 | | | | | 1.85) |
| 40 | 40,834,631 | 99,926,498 | 11 | 1 | 3.29E−09 | 1.55 (1.34, |
| | | | | | | 1.80) |
| 41 | 203,052,256 | 145,701,718 | 38 | 1 | 4.43E−07 | 1.55 (1.31, |
| | | | | | | 1.84) |
| 42 | | 32,146,043 | 9 | −1 | 4.49E−08 | 1.55 (1.33, |
| | | | | | | 1.82) |
| 43 | 89,787,193 | 144,152,322 | 15 | 1 | 2.25E−08 | 1.55 (1.33, |
| | | | | | | 1.81) |
| 44 | 87,586,162 | 200,884,864 | 9 | 1 | 1.05E−06 | 1.54 (1.29, |
| | | | | | | 1.83) |
| 45 | | 176,727,214 | 7 | 1 | 1.21E−09 | 1.53 (1.34, |
| | | | | | | 1.76) |
| 46 | | 24,110,141 | 6 | 1 | 9.06E−07 | 1.53 (1.29, |
| | | | | | | 1.82) |
| 47 | 12,764,830 | 66,597,095 | 29 | 1 | 9.27E−11 | 1.53 (1.34, |
| | | | | | | 1.73) |
| 48 | 123,109,970 | 70,719,954 | 30 | 1 | 1.88E−06 | 1.52 (1.28, |
| | | | | | | 1.80) |
| 49 | | 100,417,978 | 6 | 1 | 1.85E−06 | 1.51 (1.28, |
| | 185,265,521 | | | | | 1.79) |
| 50 | | 90,114,191 | 14 | 1 | 2.47E−06 | 1.51 (1.27, |
| | 175,223,609 | | | | | 1.80) |
| 51 | 18,270,415 | 33,468,196 | 10 | 1 | 2.73E−06 | 1.51 (1.27, |
| | 57,232,859 | | | | | 1.79) |
| 52 | | 54,197,977 | 13 | 1 | 2.46E−06 | 1.51 (1.27, |
| | | | | | | 1.79) |
| 53 | | 26,644,756 | 8 | −1 | 3.19E−06 | 1.50 (1.27, |
| | | | | | | 1.78) |
| 54 | 46,614,467 | 77,787,225 | 8 | −1 | 3.15E−06 | 1.50 (1.26, |
| | | | | | | 1.78) |
| 55 | | 119,683,295 | 7 | −1 | 8.07E−07 | 1.50 (1.27, |
| | | | | | | 1.76) |
| 56 | | 57,533,265 | 36 | 1 | 1.79E−06 | 1.47 (1.26, |
| | 69,509,878 | | | | | 1.73) |
| 57 | 84,509,965 | 107,780,779 | 7 | 1 | 7.3138E−06 | 1.47 (1.24, |
| | | | | | | 1.74) |
| 58 | | 21,721,079 | 6 | −1 | 1.09895E−05 | 1.47 (1.24, |
| | 6,633,499 | | | | | 1.75) |
| 59 | | 154,741,196 | 6 | −1 | 1.12747E−05 | 1.47 (1.24, |
| | 28,974,613 | | | | | 1.75) |
| 60 | | 44,069,502 | 9 | 1 | 0.000011555 | 1.47 (1.24, |
| | 20,959,947 | | | | | 1.75) |
| 61 | | 35,854,844 | 9 | −1 | 1.07092E−05 | 1.47 (1.24, |
| | 65,016,619 | | | | | 1.74) |
| 62 | | 7,979,363 | 14 | −1 | 0.000012473 | 1.47 (1.24, |
| | 113,217,047 | | | | | 1.74) |
| 63 | 766,337 | 28,885,534 | 26 | −1 | 8.71674E−06 | 1.47 (1.24, |
| | | | | | | 1.74) |
| 64 | | 31,519,706 | 10 | −1 | 1.47838E−05 | 1.46 (1.23, |
| | | | | | | 1.73) |
| 65 | | 7,069,800 | 15 | −1 | 2.05958E−05 | 1.45 (1.22, |
| | | | | | | 1.72) |
| 66 | 79,644,135 | 38,100,595 | 10 | −1 | 2.32E−05 | 1.45 (1.22, |
| | 17,546,317 | | | | | 1.72) |
| 67 | 143,548,460 | 92,572,965 | 6 | −1 | 2.51442E−05 | 1.45 (1.22, |
| | | | | | | 1.72) |
| 68 | 40,674,921 | 41,934,635 | 6 | −1 | 2.32037E−05 | 1.44 (1.22, |
| | | | | | | 1.71) |
| 69 | 19,408,924 | 128,413,477 | 7 | 1 | 3.19031E−05 | 1.44 (1.21, |
| | | | | | | 1.71) |
| 70 | 3,762,664 | 116,191,879 | 6 | −1 | 3.05161E−05 | 1.44 (1.21, |
| | | | | | | 1.71) |
| 71 | | 153,643,504 | 6 | 1 | 4.4005E−08 | 1.43 (1.26, |
| | 3,160,460 | | | | | 1.63) |
| 72 | | 156,841,558 | 7 | 1 | 2.77987E−05 | 1.43 (1.21, |
| | | | | | | 1.69) |
| 73 | | 47,736,928 | 6 | −1 | 5.36479E−05 | 1.42 (1.20, |
| | | | | | | 1.69) |
| 74 | 29,831,714 | 35,104,253 | 6 | 1 | 6.33162E−05 | 1.42 (1.19, |
| | | | | | | 1.68) |
| 75 | 433,778 | 1,228,434 | 6 | −1 | 0.000141468 | 1.40 (1.18, |
| | | | | | | 1.66) |
| 76 | | 51,238,065 | 8 | 1 | 2.69608E−08 | 1.39 (1.24, |
| | 32,192,717 | | | | | 1.57) |
| 77 | | 117,156,404 | 7 | −1 | 0.000186645 | 1.37 (1.16, |
| | 81,726,993 | | | | | 1.62) |
| 78 | 40,353,962 | 52,861,213 | 18 | 1 | 7.254E−07 | 1.33 (1.19, |
| | 112,063,198 | | | | | 1.49) |
| 79 | 104,161,162 | 75,467,387 | 6 | 1 | 0.001052617 | 1.33 (1.12, |
| | 152,381,718 | | | | | 1.58) |
| | 140,222,675 | | | | | |
| | 35,965,611 | | | | | |
| | 5,726,800 | | | | | |
| | 507,496 | | | | | |
| | 46,692,637 | | | | | |
| | 111,807,926 | | | | | |
| | 45,694,518 | | | | | |
| | 71,762,904 | | | | | |

TABLE 5B
Accession No. and Location of Introns in Strings based on Introns Identified in the Providence Dataset

| String No. | Intron | Accession No. | Chr. No. | Location |
|---|---|---|---|---|
| 1 | EFNA5 | NM_001962 | chr5 | 106717078-106722934, 106723017-106723405, 106723473-106762916, 106763211-107006188, |
| 1 | FAM170A | NM_182761 | chr5 | 118965534-118968441, 118968584-118969653, 118970430-118970724, 118970782-118971393, |
| 1 | FAM170A | NM_001163991 | chr5 | 118965534-118969653, 118970430-118970724, 118970782-118971393, |
| 1 | LOX | NM_002317 | chr5 | 121402445-121405746, 121405864-121406187, 121406285-121409706, 121409865- |
| | | | | 121411097, 121411237-121412586, 121412697-121413048, |
| 1 | LOX | NM_001178102 | chr5 | 121402445-121405746, 121405864-121406187, 121406285-121409706, 121409865- |
| | | | | 121411097, 121411237-121412586, |
| 1 | ALDH7A1 | NM_001182 | chr5 | 125880712-125882014, 125882092-125885619, 125885695-125885886, 125885986- |
| | | | | 125887711, 125887830-125889977, 125890086-125891621, 125891708-125894930, 125895027- |
| | | | | 125896773, 125896817-125903949, 125904049-125906472, 125906552-125911110, 125911157- |
| | | | | 125912769, 125912904-125918541, 125918667-125919622, 125919705-125928354, 125928422- |
| | | | | 125929041, 125929097-125930697, |
| 1 | PRRC1 | NM_130809 | chr5 | 126853469-126859150, 126859275-126860221, 126860613-126862308, 126862471- |
| | | | | 126865984, 126866089-126869313, 126869479-126874730, 126874836-126883509, 126883614- |
| | | | | 126887397, |
| 1 | SEC24A | NM_021982 | chr5 | 133984864-133996807, 133997277-134002511, 134002687-134007497, 134007577- |
| | | | | 134010303, 134010466-134010733, 134010908-134011711, 134011816-134015290, 134015419- |
| | | | | 134018061, 134018173-134022478, 134022593-134023869, 134023990-134028290, 134028348- |
| | | | | 134029415, 134029624-134032814, 134032937-134033587, 134033748-134039447, 134039623- |
| | | | | 134041015, 134041128-134044401, 134044579-134050712, 134050852-134053757, 134053864- |
| | | | | 134056686, 134056781-134059255, 134059361-134060668, |
| 1 | TRPC7 | NM_001167577 | chr5 | 135549293-135551886, 135551964-135560960, 135561043-135561720, 135561944- |
| | | | | 135567040, 135567238-135583157, 135583424-135587335, 135587571-135601906, 135602125- |
| | | | | 135610359, 135610526-135692294, 135693074-135700939, |
| 1 | TRPC7 | NM_020389 | chr5 | 135549293-135551886, 135551964-135560960, 135561043-135561720, 135561944- |
| | | | | 135567040, 135567238-135583157, 135583424-135587335, 135587571-135601906, 135602125- |
| | | | | 135610359, 135610526-135651283, 135651468-135692294, 135693074-135700939, |
| 1 | TRPC7 | NM_001167576 | chr5 | 135549293-135551886, 135551964-135560960, 135561043-135561720, 135561944- |
| | | | | 135567040, 135567238-135583157, 135583424-135587335, 135587571-135601906, 135602125- |
| | | | | 135692294, 135693074-135700939, |
| 1 | CDC25C | NM_022809 | chr5 | 137621531-137621709, 137621823-137622184, 137622320-137622856, 137622957- |
| | | | | 137625188, 137625253-137626326, 137626430-137627657, 137627806-137654906, 137655064- |
| | | | | 137664171, 137664219-137666674, 137666908-137667282, |
| 1 | CDC25C | NM_001790 | chr5 | 137621531-137621709, 137621823-137622184, 137622320-137622856, 137622957- |
| | | | | 137625188, 137625253-137626326, 137626430-137627657, 137627806-137654906, 137655064- |
| | | | | 137661502, 137661594-137661708, 137661744-137664171, 137664219-137665240, 137665337- |
| | | | | 137666674, 137666908-137667282, |
| 1 | HSPA9 | NM_004134 | chr5 | 137891804-137892139, 137892282-137892461, 137892556-137893089, 137893186- |
| | | | | 137893556, 137893676-137894240, 137894347-137895551, 137895781-137897267, 137897479- |
| | | | | 137902313, 137902408-137902688, 137902853-137903142, 137903251-137903336, 137903412- |
| | | | | 137904612, 137904739-137906647, 137906831-137909450, 137909540-137909755, 137909816- |
| | | | | 137910925, |
| 1 | NRG2 | NM_013983 | chr5 | 139228283-139231187, 139231417-139232015, 139232145-139232487, 139232613- |
| | | | | 139235259, 139235364-139239472, 139239498-139244698, 139244759-139251304, 139251427- |
| | | | | 139260439, 139260560-139266923, 139267097-139421953, |
| 1 | NRG2 | NM_013981 | chr5 | 139228283-139231187, 139231417-139232015, 139232145-139232487, 139232613- |
| | | | | 139235259, 139235364-139244698, 139244759-139251304, 139251427-139260439, 139260560- |
| | | | | 139266923, 139267097-139421953, |
| 1 | NRG2 | NM_001184935 | chr5 | 139228283-139231187, 139231417-139232015, 139232145-139232487, 139232613- |
| | | | | 139235259, 139235364-139260439, 139260560-139266923, 139267097-139421953, |
| 1 | NRG2 | NM_013982 | chr5 | 139228283-139231187, 139231417-139232015, 139232145-139232487, 139232613- |
| | | | | 139235259, 139235364-139239472, 139239498-139245132, 139245211-139251304, 139251427- |
| | | | | 139260439, 139260560-139266923, 139267097-139421953, |
| 1 | NRG2 | NM_004883 | chr5 | 139228283-139231187, 139231417-139232015, 139232145-139232487, 139232613- |
| | | | | 139235259, 139235364-139245132, 139245211-139251304, 139251427-139260439, 139260560- |
| | | | | 139266923, 139267097-139421953, |
| 1 | PCDHGA7 | NM_018920 | chr5 | 140764891-140874372, 140874433-140884958, 140885049-140890512, |
| 1 | PCDHGA6 | NM_018919 | chr5 | 140756075-140874372, 140874433-140884958, 140885049-140890512, |
| 1 | PCDHGB4 | NM_003736 | chr5 | 140769849-140874372, 140874433-140884958, 140885049-140890512, |
| 1 | DIAPH1 | NM_005219 | chr5 | 140896576-140903708, 140903797-140905603, 140905741-140905862, 140906029- |
| | | | | 140907138, 140907265-140908018, 140908150-140908267, 140908509-140908737, 140908841- |
| | | | | 140909168, 140909265-140913900, 140914001-140915619, 140915630-140951491, 140951608- |
| | | | | 140953057, 140953776-140954532, 140954714-140955795, 140955862-140956321, 140956439- |
| | | | | 140957040, 140957159-140957790, 140957911-140958080, 140958193-140958653, 140958764- |
| | | | | 140960309, 140960451-140961877, 140961943-140962771, 140962860-140963050, 140963183- |
| | | | | 140963689, 140963793-140966607, 140966765-140967789, 140967818-140998363, |
| 1 | DIAPH1 | NM_001079812 | chr5 | 140896576-140903708, 140903797-140905603, 140905741-140905862, 140906029- |
| | | | | 140907138, 140907265-140908018, 140908150-140908267, 140908509-140908737, 140908841- |
| | | | | 140909168, 140909265-140913900, 140914001-140915619, 140915630-140951491, 140951608- |
| | | | | 140953057, 140953776-140954532, 140954714-140955795, 140955862-140956321, 140956439- |
| | | | | 140957040, 140957159-140957790, 140957911-140958080, 140958193-140958653, 140958764- |
| | | | | 140960309, 140960451-140961877, 140961943-140962771, 140962860-140963050, 140963183- |
| | | | | 140963689, 140963793-140966607, 140966765-140998363, |
| 2 | SLC34A1 | NM_003052 | chr5 | 176811489-176812694, 176812852-176812986, 176813138-176813220, 176813351- |
| | | | | 176813422, 176813568-176814761, 176814875-176814993, 176815191-176815276, 176815374- |
| | | | | 176820693, 176820765-176821027, 176821197-176823732, 176823851-176823949, 176824076- |
| | | | | 176824782, |
| 2 | SLC34A1 | NM_001167579 | chr5 | 176811489-176812694, 176812852-176812986, 176813138-176813220, 176813351- |
| | | | | 176813422, 176813568-176814761, 176814875-176814993, 176815191-176815276, 176815374- |
| | | | | 176816638, |
| 2 | PRR7 | NM_030567 | chr5 | 176873962-176880955, 176881042-176881828, 176882496-176882723, |
| 2 | PRR7 | NM_001174101 | chr5 | 176873962-176881828, 176882496-176882723, |
| 2 | PRR7 | NM_001174102 | chr5 | 176875409-176881828, 176882496-176882723, |
| 2 | AACSP1 | NR_024035 | chr5 | 178193177-178194129, 178194337-178195469, 178195666-178199428, 178199633- |
| | | | | 178201497, 178201550-178202266, 178202338-178203141, |
| 2 | CANX | NM_001746 | chr5 | 179126104-179132678, 179132854-179133257, 179133333-179134131, 179134192- |
| | | | | 179135238, 179135382-179135977, 179136061-179136872, 179137067-179143104, 179143296- |
| | | | | 179146667, 179146783-179147403, 179147562-179149803, 179150021-179150659, 179150781- |
| | | | | 179151656, 179151785-179153679, 179153761-179155590, |
| 2 | CANX | NM_001024649 | chr5 | 179126051-179132678, 179132854-179133257, 179133333-179134131, 179134192- |
| | | | | 179135238, 179135382-179135977, 179136061-179136872, 179137067-179143104, 179143296- |
| | | | | 179146667, 179146783-179147403, 179147562-179149803, 179150021-179150659, 179150781- |
| | | | | 179151656, 179151785-179153679, 179153761-179155590, |
| 2 | MAML1 | NM_014757 | chr5 | 179160429-179192325, 179193743-179195849, 179196091-179198146, 179198245-179200894, |
| 2 | SQSTM1 | NM_003900 | chr5 | 179248142-179249956, 179250054-179250856, 179251088-179251180, 179251324- |
| | | | | 179252144, 179252227-179260030, 179260247-179260585, 179260783-179263434, |
| 2 | SQSTM1 | NM_001142299 | chr5 | 179234124-179238572, 179238683-179249956, 179250054-179250856, 179251088- |
| | | | | 179251180, 179251324-179252144, 179252227-179260030, 179260247-179260585, 179260783- |
| | | | | 179263434, |
| 2 | SQSTM1 | NM_001142298 | chr5 | 179233592-179238572, 179238683-179249956, 179250054-179250856, 179251088- |
| | | | | 179251180, 179251324-179252144, 179252227-179260030, 179260247-179260585, 179260783- |
| | | | | 179263434, |
| 2 | RASGEF1C | NM_175062 | chr5 | 179528526-179529069, 179529144-179538455, 179538581-179541444, 179541542- |
| | | | | 179545607, 179545705-179545785, 179545867-179546344, 179546449-179548058, 179548150- |
| | | | | 179554607, 179554684-179555408, 179555611-179563376, 179563516-179564588, 179564713- |
| | | | | 179564874, 179565059-179636026, |
| 2 | GNB2L1 | NM_006098 | chr5 | 180664043-180664607, 180664720-180665097, 180665240-180666065, 180666178- |
| | | | | 180666485, 180666583-180668490, 180668640-180669172, 180669346-180670690, |
| 3 | RAD52 | NM_134424 | chr12 | 1022619-1023058, 1023288-1023595, 1023699-1025508, 1025650-1025803, 1025987-1034614, 1034692- |
| | | | | 1036309, 1036430-1038983, 1039053-1039215, 1039311-1040384, 1040488-1042139, 1042243-1058766, |
| 3 | DCP1B | NM_152640 | chr12 | 2055453-2058250, 2058501-2061580, 2062455-2064596, 2064727-2074722, 2074860-2077008, 2077077- |
| | | | | 2102428, 2102558-2107099, 2107142-2113446, |
| 3 | PARP11 | NM_020367 | chr12 | 3921606-3923201, 3923355-3931037, 3931170-3931249, 3931324-3935322, 3935400-3938074, 3938197- |
| | | | | 3939054, 3939185-3982376, |
| 3 | C12orf5 | NM_020375 | chr12 | 4430470-4440444, 4440484-4446203, 4446327-4458983, 4459063-4460431, 4460544-4461424, |
| 3 | VAMP1 | NM_014231 | chr12 | 6573653-6574054, 6574108-6575006, 6575167-6575389, 6575518-6579694, |
| 3 | VAMP1 | NM_016830 | chr12 | 6572009-6574054, 6574108-6575006, 6575167-6575389, 6575518-6579694, |
| 3 | VAMP1 | NM_199245 | chr12 | 6574108-6575006, 6575167-6575389, 6575518-6579694, |
| 3 | ACRBP | NM_032489 | chr12 | 6747571-6748118, 6748204-6749224, 6749394-6749507, 6749689-6752703, 6752838-6753301, 6753772- |
| | | | | 6754384, 6754504-6755647, 6755744-6755958, 6756179-6756488, |
| 3 | ZNF384 | NM_001039918 | chr12 | 6777282-6778195, 6778356-6779924, 6780005-6782380, 6782607-6787291, 6787627-6788110, 6788350- |
| | | | | 6788619, 6788692-6797331, 6797393-6798262, |
| 3 | ZNF384 | NM_001039920 | chr12 | 6777282-6778195, 6778356-6779924, 6780005-6782380, 6782607-6787291, 6787510-6788110, 6788350- |
| | | | | 6788619, 6788692-6797331, 6797393-6798532, |
| 3 | ZNF384 | NM_001135734 | chr12 | 6777282-6778195, 6778356-6779924, 6780005-6781514, 6781699-6782380, 6782607-6787291, 6787627- |
| | | | | 6787827, 6787877-6788110, 6788350-6788619, 6788692-6797331, 6797393-6798532, |
| 3 | ZNF384 | NM_001039916 | chr12 | 6777282-6778195, 6778356-6779924, 6780005-6782380, 6782607-6787291, 6787627-6787827, 6787877- |
| | | | | 6788110, 6788350-6788619, 6788692-6797331, |
| 3 | ZNF384 | NM_001039917 | chr12 | 6777282-6778195, 6778356-6779924, 6780005-6782380, 6782607-6787291, 6787627-6787827, 6787877- |
| | | | | 6788110, 6788350-6788619, 6788692-6797331, 6797393-6798532, |
| 3 | ZNF384 | NM_133476 | chr12 | 6777282-6778195, 6778356-6779924, 6780005-6782380, 6782607-6787291, 6787627-6787827, 6787877- |
| | | | | 6788110, 6788350-6788619, 6788692-6797331, 6797393-6798262, |
| 3 | ZNF384 | NM_001039919 | chr12 | 6777282-6778195, 6778356-6779924, 6780005-6782380, 6782607-6787291, 6787627-6788110, 6788350- |
| | | | | 6788619, 6788692-6797331, 6797393-6798532, |
| 3 | C12orf53 | NM_153685 | chr12 | 6804224-6804594, 6804818-6805607, 6805691-6806451, 6806959-6807209, 6807271-6809778, |
| 3 | LAG3 | NM_002286 | chr12 | 6882077-6882356, 6882506-6882861, 6883168-6883759, 6884031-6884437, 6884715-6886428, 6886673- |
| | | | | 6886955, 6887088-6887408, |
| 3 | LRRC23 | NM_006992 | chr12 | 7014068-7014747, 7014924-7015007, 7015119-7015571, 7015827-7016477, 7016610-7019052, 7019191- |
| | | | | 7023053, |
| 3 | LRRC23 | NM_201650 | chr12 | 7014068-7014747, 7014924-7015007, 7015119-7015571, 7015827-7016477, 7016610-7019052, 7019191- |
| | | | | 7021892, 7022192-7023053, |
| 3 | LRRC23 | NM_001135217 | chr12 | 7014240-7014747, 7014924-7015007, 7015119-7015571, 7015827-7016477, 7016610-7019052, 7019191- |
| | | | | 7021892, 7022192-7023053, |
| 3 | ENO2 | NM_001975 | chr12 | 7023824-7024983, 7025082-7025579, 7025677-7025833, 7025894-7026200, 7026272-7026743, 7026879- |
| | | | | 7027102, 7027327-7028728, 7028928-7030742, 7030946-7031217, 7031328-7031505, 7031566-7031892, |
| 3 | ATN1 | NM_001007026 | chr12 | 7033701-7043001, 7043192-7043337, 7043477-7043626, 7043742-7044708, 7046725-7047006, 7047231- |
| | | | | 7047642, 7048341-7050041, 7050187-7050535, 7050718-7050908, |
| 3 | ATN1 | NM_001940 | chr12 | 7037548-7043001, 7043192-7043337, 7043477-7043626, 7043742-7044708, 7046725-7047006, 7047231- |
| | | | | 7047642, 7048341-7050041, 7050187-7050535, 7050718-7050908, |
| 3 | C12orf57 | NM_138425 | chr12 | 7053337-7053637, 7053816-7054932, |
| 3 | FOXJ2 | NM_018416 | chr12 | 8186430-8192413, 8192762-8195252, 8195329-8196295, 8196366-8196545, 8196688-8197354, 8197555- |
| | | | | 8200476, 8200886-8201291, 8201395-8201956, 8202168-8203116, 8203217-8205356, |
| 3 | NECAP1 | NM_015509 | chr12 | 8234980-8242530, 8242633-8242789, 8242896-8244363, 8244447-8245270, 8245381-8245466, 8245652- |
| | | | | 8248195, 8248300-8248636, |
| 3 | NECAP1 | NR_024260 | chr12 | 8234980-8242530, 8242633-8242789, 8242896-8245270, 8245381-8245466, 8245652-8248195, 8248300- |
| | | | | 8248636, |
| 3 | CLEC4A | NM_016184 | chr12 | 8276557-8278155, 8278274-8281858, 8281959-8288179, 8288333-8289382, 8289500-8290734, |
| 3 | CLEC4A | NM_194450 | chr12 | 8276557-8278155, 8278274-8288179, 8288333-8289382, 8289500-8290734, |
| 3 | CLEC4A | NM_194448 | chr12 | 8276557-8288179, 8288333-8289382, 8289500-8290734, |
| 3 | CLEC4A | NM_194447 | chr12 | 8276557-8281858, 8281959-8288179, 8288333-8289382, 8289500-8290734, |
| 3 | FAMGGC | NR_026788 | chr12 | 8333349-8340326, 8340822-8341066, 8341242-8346114, 8346252-8346797, 8347078-8347307, 8347393- |
| | | | | 8348851, 8349115-8352302, |
| 4 | ASTN2 | NM_001184735 | chr9 | 119375885-119380601, 119380752-119382587, 119382723-119413805, 119414073-119449343, |
| 4 | ASTN2 | NM_198188 | chr9 | 119187906-119188153, 119188368-119202886, 119203072-119204730, 119204833- |
| | | | | 119249636, 119249780-119380601, 119380752-119382587, 119382723-119413805, 119414073- |
| | | | | 119449343, |
| 4 | ASTN2 | NM_001184734 | chr9 | 119187906-119188187, 119188368-119202886, 119203072-119204730, 119204833- |
| | | | | 119249636, 119249780-119380601, 119380752-119382587, 119382723-119413805, 119414073- |
| | | | | 119449343, |
| 4 | ASTN2 | NM_014010 | chr9 | 119188368-119202886, 119203072-119204730, 119204833-119249636, 119249780- |
| | | | | 119380601, 119380752-119382587, 119382723-119413805, 119414073-119488048, 119488230- |
| | | | | 119491269, 119491376-119495676, 119495803-119567909, 119568100-119582894, 119583063- |
| | | | | 119625860, 119626013-119737485, 119737625-119738391, 119738468-119738978, 119739065- |
| | | | | 119770369, 119770539-119802096, 119802245-119858321, 119858431-119976635, 119977022- |
| | | | | 120053603, 120053793-120176773, |
| 4 | ASTN2 | NM_198187 | chr9 | 119187906-119188132, 119188368-119202886, 119203072-119204730, 119204833- |
| | | | | 119249636, 119249780-119380601, 119380752-119382587, 119382723-119413805, 119414073- |
| | | | | 119449343, |
| 4 | ASTN2 | NM_198186 | chr9 | 119188368-119202886, 119203072-119204730, 119204833-119249636, 119249780- |
| | | | | 119380601, 119380752-119382587, 119382723-119413805, 119414073-119448974, |
| 4 | TRIM32 | NM_001099679 | chr9 | 119449661-119459942, |
| 4 | TRIM32 | NM_012210 | chr9 | 119449661-119459939, |
| 4 | MEGF9 | NM_001080497 | chr9 | 123367920-123370017, 123370289-123374672, 123374818-123384892, 123385034- |
| | | | | 123421650, 123421854-123476034, |
| 4 | LOC100288842 | NR_027442 | chr9 | 123556201-123560851, |
| 4 | C5 | NM_001735 | chr9 | 123715147-123716006, 123716147-123719561, 123719647-123722524, 123722616- |
| | | | | 123723758, 123723844-123724947, 123725055-123725169, 123725246-123725912, 123726017- |
| | | | | 123731337, 123731397-123732446, 123732530-123734206, 123734271-123737055, 123737210- |
| | | | | 123738976, 123739184-123742359, 123742533-123744120, 123744218-123744931, 123745093- |
| | | | | 123751322, 123751400-123751844, 123752059-123753467, 123753559-123758516, 123758579- |
| | | | | 123759823, 123760053-123768195, 123768337-123769180, 123769256-123770684, 123770777- |
| | | | | 123776149, 123776349-123777475, 123777540-123778530, 123778662-123779638, 123779790- |
| | | | | 123779919, 123780131-123782252, 123782458-123783785, 123783973-123785680, 123785798- |
| | | | | 123787730, 123787859-123789436, 123789553-123792673, 123792766-123794389, 123794474- |
| | | | | 123797079, 123797173-123800157, 123800230-123805280, 123805445-123808467, 123808662- |
| | | | | 123812458, |
| 4 | RAB14 | NM_016322 | chr9 | 123943852-123945206, 123945239-123945587, 123945677-123949229, 123949298- |
| | | | | 123952830, 123953010-123954447, 123954503-123955637, 123955698-123963915, |
| 4 | ZBTB26 | NM_020924 | chr9 | 125682224-125693714, |
| 4 | ZBTB34 | NM_001099270 | chr9 | 129623019-129641667, |
| 4 | RALGPS1 | NM_014636 | chr9 | 129677255-129724502, 129724626-129728121, 129728231-129739972, 129740025- |
| | | | | 129796708, 129796794-129812320, 129812412-129815124, 129815219-129831507, 129831636- |
| | | | | 129928346, 129928486-129930956, 129931052-129936992, 129937062-129957368, 129957497- |
| | | | | 129958751, 129958911-129961292, 129961346-129974408, 129974516-129974904, 129974999- |
| | | | | 129975221, 129975329-129977028, 129977122-129981017, |
| 4 | RALGPS1 | NM_001190728 | chr9 | 129724626-129728121, 129728231-129739972, 129740025-129796708, 129796794- |
| | | | | 129812320, 129812412-129815124, 129815219-129831507, 129831636-129928346, 129928486- |
| | | | | 129930956, 129931052-129936992, 129937062-129957368, 129957497-129972972, 129973058- |
| | | | | 129974408, 129974516-129974904, 129974999-129975245, 129975329-129977028, 129977122- |
| | | | | 129979183, |
| 4 | RALGPS1 | NM_001190729 | chr9 | 129677292-129724502, 129724626-129728121, 129728231-129739972, 129740025- |
| | | | | 129796708, 129796794-129812320, 129812412-129815124, 129815219-129831507, 129831636- |
| | | | | 129928346, 129928486-129930956, 129931052-129936992, 129937062-129957368, 129957497- |
| | | | | 129972972, 129973058-129974408, 129974516-129974904, 129974999-129975221, 129975329- |
| | | | | 129977028, 129977122-129979183, |
| 4 | RALGPS1 | NM_001190730 | chr9 | 129677292-129724502, 129724626-129728121, 129728231-129739972, 129740025- |
| | | | | 129796708, 129796794-129812320, 129812412-129815124, 129815219-129831507, 129831636- |
| | | | | 129928346, 129928486-129930956, 129931052-129936992, 129937062-129940174, |
| 4 | SLC2A8 | NM_014580 | chr9 | 130159566-130159653, 130159818-130160182, 130160391-130162184, 130162286- |
| | | | | 130164834, 130165033-130165937, 130166083-130166236, 130166347-130167095, 130167271- |
| | | | | 130167697, 130167845-130169389, |
| 4 | ZNF79 | NM_007135 | chr9 | 130187083-130191109, 130191200-130197367, 130197496-130198185, 130198283-130206306, |
| 4 | C9orf117 | NM_001012502 | chr9 | 130469472-130471699, 130471973-130472878, 130473034-130473506, 130473776- |
| | | | | 130474097, 130474230-130474476, 130474629-130474986, 130475155-130475297, 130475486- |
| | | | | 130476111, |
| 5 | HMGCR | NM_000859 | chr5 | 74633126-74638406, 74638596-74639676, 74639790-74640068, 74640158-74641397, 74641484- |
| | | | | 74643027, 74643135-74645865, 74645974-74646081, 74646200-74646612, 74646775-74646891, 74647141- |
| | | | | 74647247, 74647428-74650326, 74650523-74650879, 74651040-74651188, 74651348-74652166, 74652274- |
| | | | | 74654480, 74654653-74654993, 74655136-74655221, 74655382-74655808, 74655965-74656111, |
| 5 | HMGCR | NM_001130996 | chr5 | 74633126-74638406, 74638596-74639676, 74639790-74640068, 74640158-74641397, 74641484- |
| | | | | 74643027, 74643135-74645865, 74645974-74646081, 74646200-74646612, 74646775-74646891, 74647141- |
| | | | | 74647247, 74647428-74650326, 74650523-74651188, 74651348-74652166, 74652274-74654480, 74654653- |
| | | | | 74654993, 74655136-74655221, 74655382-74655808, 74655965-74656111, |
| 5 | OTP | NM_032109 | chr5 | 76926620-76932644, 76933056-76934336, |
| 5 | HOMER1 | NM_004272 | chr5 | 78672021-78692644, 78692727-78693348, 78693461-78697720, 78697879-78734831, 78734973- |
| | | | | 78742874, 78742969-78746811, 78746945-78752683, 78752842-78808591, |
| 5 | ARRDC3 | NM_020801 | chr5 | 90667274-90669499, 90669656-90669929, 90670094-90670737, 90670996-90671326, 90671431- |
| | | | | 90672436, 90672586-90674509, 90674593-90678628, |
| 5 | LIX1 | NM_153234 | chr5 | 96430740-96432512, 96432592-96440892, 96440990-96443062, 96443205-96460168, 96460334-96478197, |
| 5 | SLCO6A1 | NM_173488 | chr5 | 101707828-101709039, 101709199-101724390, 101724530-101726683, 101726750- |
| | | | | 101735257, 101735447-101748692, 101748848-101755528, 101755726-101774319, 101774466- |
| | | | | 101794084, 101794196-101795358, 101795482-101811399, 101811498-101813378, 101813566- |
| | | | | 101815879, 101816139-101834189, |
| 6 | DIO1 | NM_213593 | chr1 | 54360029-54370337, 54370483-54371766, 54371968-54375602, |
| 6 | DIO1 | NM_001039716 | chr1 | 54360221-54370337, 54370483-54375602, |
| 6 | DIO1 | NM_001039715 | chr1 | 54360221-54371766, 54371968-54375602, |
| 6 | DIO1 | NM_000792 | chr1 | 54360221-54370337, 54370483-54371766, 54371968-54375602, |
| 6 | LRRC42 | NM_052940 | chr1 | 54412223-54417657, 54418146-54423820, 54423954-54426027, 54426148-54427705, 54427796- |
| | | | | 54427967, 54428083-54431967, 54432054-54433336, |
| 6 | C1orf168 | NM_001004303 | chr1 | 57185366-57185693, 57185729-57185844, 57186003-57189259, 57189355-57192168, 57192257- |
| | | | | 57202758, 57202821-57204296, 57204327-57206368, 57206469-57207832, 57207895-57209697, 57209740- |
| | | | | 57209823, 57209940-57216715, 57216877-57219510, 57219609-57221567, 57221601-57224387, 57224424- |
| | | | | 57233500, 57233612-57252846, 57252882-57254644, 57254808-57257727, 57258477-57284953, |
| 6 | DAB1 | NM_021080 | chr1 | 57463802-57476351, 57476464-57476816, 57476946-57480554, 57481105-57489202, 57489313- |
| | | | | 57491652, 57491717-57528555, 57528617-57535031, 57535099-57536694, 57536735-57537193, 57537315- |
| | | | | 57537954, 57538088-57602214, 57602315-57610961, 57611103-57756634, 57756839-58115262, 58115338- |
| | | | | 58349669, 58349835-58715910, |
| 6 | OMA1 | NM_145243 | chr1 | 58946847-58971730, 58971882-58992931, 58993008-58996271, 58996402-58999623, 58999733- |
| | | | | 58999828, 59000004-59002183, 59002414-59004465, 59004983-59012373, |
| 6 | WDR78 | NM_024763 | chr1 | 67279864-67288042, 67288201-67292501, 67292730-67293493, 67293594-67299266, 67299390- |
| | | | | 67299672, 67299832-67301307, 67301461-67303391, 67303480-67306150, 67306355-67313165, 67313362- |
| | | | | 67327828, 67327986-67337051, 67337193-67340462, 67340621-67356835, 67356950-67358910, 67359097- |
| | | | | 67370882, 67371059-67390343, |
| 6 | WDR78 | NM_207014 | chr1 | 67303480-67306150, 67306355-67313165, 67313362-67327828, 67327986-67337051, 67337193- |
| | | | | 67340462, 67340621-67356835, 67356950-67358910, 67359097-67370882, 67371059-67390343, |
| 6 | SLC35D1 | NM_015139 | chr1 | 67470132-67474766, 67474851-67486050, 67486131-67487216, 67487286-67507917, 67508012- |
| | | | | 67512946, 67513051-67515463, 67515534-67516114, 67516188-67517693, 67517763-67518452, 67518541- |
| | | | | 67518637, 67518673-67519492, |
| 6 | HHLA3 | NM_001031693 | chr1 | 70820795-70832125, 70832221-70833298, |
| 6 | HHLA3 | NM_001036645 | chr1 | 70820892-70832125, 70832221-70833298, |
| 6 | HHLA3 | NM_001036646 | chr1 | 70820791-70832125, 70832221-70833298, |
| 6 | HHLA3 | NR_027404 | chr1 | 70820791-70832125, 70832221-70832614, 70832668-70833298, |
| 6 | PTGER3 | NM_001126044 | chr1 | 71418743-71419444, 71419473-71439978, 71440072-71477986, 71478168-71512362, |
| 6 | PTGER3 | NM_198716 | chr1 | 71318543-71419444, 71419473-71477986, 71478168-71512362, |
| 6 | PTGER3 | NM_198718 | chr1 | 71418743-71419444, 71419473-71477986, 71478168-71512362, |
| 6 | PTGER3 | NM_198715 | chr1 | 71478168-71512362, |
| 6 | PTGER3 | NM_198719 | chr1 | 71437417-71439978, 71440072-71477986, 71478168-71512362, |
| 6 | PTGER3 | NM_198714 | chr1 | 71318543-71419444, 71419473-71439978, 71440072-71477986, 71478168-71512362, |
| 6 | PTGER3 | NR_028292 | chr1 | 71318543-71327986, 71328081-71334925, 71335003-71419444, 71419473-71477986, 71478168-71512362, |
| 6 | PTGER3 | NR_028293 | chr1 | 71318543-71327986, 71328081-71334925, 71335003-71477986, 71478168-71512362, |
| 6 | PTGER3 | NM_198717 | chr1 | 71318543-71477986, 71478168-71512362, |
| 6 | PTGER3 | NR_028294 | chr1 | 71318543-71327986, 71328081-71331370, 71331517-71419444, 71419473-71477986, 71478168-71512362, |
| 6 | NEGR1 | NM_173808 | chr1 | 71873254-72058498, 72058652-72076707, 72076830-72163689, 72163823-72241853, 72241981- |
| | | | | 72400760, 72400995-72748000, |
| 6 | C1orf173 | NM_001002912 | chr1 | 75036124-75036781, 75039176-75042577, 75042621-75055313, 75055765-75065377, 75065616- |
| | | | | 75072283, 75072587-75078305, 75078494-75086416, 75086599-75097395, 75097613-75101962, 75102123- |
| | | | | 75107013, 75107144-75108709, 75108783-75112349, 75112477-75114904, 75115000-75139179, |
| 6 | CRYZ | NM_001130042 | chr1 | 75172142-75172581, 75172679-75172785, 75172889-75175780, 75175932-75180261, 75180315- |
| | | | | 75184891, 75185057-75188800, 75188955-75190393, 75190519-75196064, 75196105-75198638, |
| 6 | CRYZ | NM_001130043 | chr1 | 75172142-75172581, 75172679-75175780, 75175932-75180261, 75180315-75184891, 75185057- |
| | | | | 75188800, 75188955-75190393, 75190519-75198638, |
| 6 | CRYZ | NM_001889 | chr1 | 75172142-75172581, 75172679-75172785, 75172889-75175780, 75175932-75180261, 75180315- |
| | | | | 75184891, 75185057-75188800, 75188955-75190393, 75190519-75198638, |
| 6 | CRYZ | NM_001134759 | chr1 | 75172142-75172581, 75172679-75172785, 75172889-75175780, 75175932-75180261, 75180315- |
| | | | | 75188800, 75188955-75198638, |
| 6 | TYW3 | NM_001162916 | chr1 | 75199103-75202224, 75202307-75214433, 75214507-75217974, 75218110-75229576, |
| 6 | TYW3 | NM_138467 | chr1 | 75199103-75202224, 75202307-75204372, 75204473-75214433, 75214507-75217974, 75218110-75229576, |
| 6 | TYW3 | NR_027962 | chr1 | 75199216-75202224, 75202307-75204372, 75204473-75214433, 75214507-75217974, 75218110-75229576, |
| 6 | FAM73A | NM_198549 | chr1 | 78245422-78248921, 78249037-78267014, 78267194-78268953, 78269092-78272658, 78272787- |
| | | | | 78279417, 78279553-78280791, 78280917-78308990, 78309093-78324621, 78324742-78324997, 78325072- |
| | | | | 78325723, 78325812-78326907, 78327008-78329577, 78329714-78332021, 78332077-78338687, 78338806- |
| | | | | 78340529, |
| 6 | RPF1 | NM_025065 | chr1 | 84945193-84946637, 84946696-84948596, 84948679-84955314, 84955412-84956000, 84956156- |
| | | | | 84961054, 84961139-84961563, 84961747-84961925, 84962054-84963110, |
| 6 | TGFBR3 | NM_001195684 | chr1 | 92149415-92161227, 92161337-92163644, 92163688-92174218, 92174341-92177798, 92178100- |
| | | | | 92181791, 92181952-92182123, 92182266-92184867, 92185022-92185448, 92185785-92187510, 92187702- |
| | | | | 92193214, 92193364-92195360, 92195531-92200331, 92200517-92224168, 92224308-92262842, 92263029- |
| | | | | 92327026, 92327202-92365192, 92365255-92371381, |
| 6 | TGFBR3 | NM_001195683 | chr1 | 92149415-92161227, 92161337-92163644, 92163688-92174218, 92174341-92177798, 92178100- |
| | | | | 92181791, 92181952-92182123, 92182266-92184867, 92185022-92185448, 92185785-92187510, 92187702- |
| | | | | 92193214, 92193364-92195360, 92195531-92200331, 92200517-92224168, 92224308-92262842, 92263029- |
| | | | | 92327026, 92327202-92351433, |
| 6 | TGFBR3 | NM_003243 | chr1 | 92149415-92161227, 92161337-92163644, 92163688-92174218, 92174341-92177798, 92178100- |
| | | | | 92181791, 92181952-92182123, 92182266-92184867, 92185022-92185448, 92185788-92187510, 92187702- |
| | | | | 92193214, 92193364-92195360, 92195531-92200331, 92200517-92224168, 92224308-92262842, 92263029- |
| | | | | 92327026, 92327202-92351433, |
| 6 | TGFBR3 | NR_036634 | chr1 | 92149415-92161227, 92161337-92163644, 92163688-92174218, 92174341-92177798, 92178100- |
| | | | | 92181791, 92181952-92182123, 92182266-92184867, 92185022-92185448, 92185788-92187510, 92187702- |
| | | | | 92193214, 92193364-92195360, 92195531-92200331, 92200517-92224168, 92224308-92262842, 92263029- |
| | | | | 92266514, 92266613-92327026, 92327202-92351433, |
| 6 | C1orf146 | NM_001012425 | chr1 | 92683606-92696937, 92697044-92707767, 92707863-92709772, 92709943-92710334, 92710415-92711095, |
| 6 | BCAR3 | NM_003567 | chr1 | 94027977-94032834, 94033049-94033295, 94033409-94037225, 94037399-94041569, 94041687- |
| | | | | 94047856, 94048511-94049573, 94049679-94054532, 94054977-94057820, 94057951-94107858, 94107900- |
| | | | | 94140168, 94140498-94147152, |
| 6 | LOC100129046 | NR_034091 | chr1 | 94058066-94059928, 94060007-94064472, 94064548-94064716, |
| 6 | ALG14 | NM_144988 | chr1 | 95448863-95492683, 95492817-95530420, 95530574-95538317, |
| 6 | CCDC76 | NM_019083 | chr1 | 100598872-100602436, 100602485-100602573, 100602642-100605203, 100605268- |
| | | | | 100605736, 100605808-100605962, 100606071-100606406, 100606576-100608691, 100608766- |
| | | | | 100609623, 100609700-100613448, 100613883-100614179, |
| 6 | SLC25A24 | NM_013386 | chr1 | 108679460-108681678, 108681831-108686163, 108686333-108690899, 108691009- |
| | | | | 108697603, 108697758-108700082, 108700243-108703802, 108703916-108724561, 108724651- |
| | | | | 108728448, 108728577-108742576, |
| 6 | SLC25A24 | NM_213651 | chr1 | 108679460-108681678, 108681831-108686163, 108686333-108690899, 108691009- |
| | | | | 108697603, 108697758-108700082, 108700243-108703802, 108703916-108724561, 108724651- |
| | | | | 108728448, 108728577-108735116, |
| 6 | TMEM167B | NM_020141 | chr1 | 109633505-109635510, 109635644-109637037, |
| 6 | KIAA1324 | NM_020775 | chr1 | 109656959-109704514, 109704637-109707119, 109707314-109714486, 109714636- |
| | | | | 109715108, 109715191-109716094, 109716202-109716308, 109716460-109727665, 109727756- |
| | | | | 109730797, 109730977-109731686, 109731817-109732212, 109732305-109733986, 109734094- |
| | | | | 109734346, 109734486-109735231, 109735497-109737041, 109737217-109740094, 109740277- |
| | | | | 109740599, 109740698-109741193, 109741297-109742474, 109742603-109742679, 109742860- |
| | | | | 109743355, 109743523-109745564, |
| 6 | SARS | NR_034073 | chr1 | 109779996-109780351, |
| 6 | SARS | NR_034072 | chr1 | 109756751-109757167, |
| 6 | SARS | NM_006513 | chr1 | 109756751-109766598, 109766671-109770972, 109771055-109772034, 109772195- |
| | | | | 109773498, 109773644-109774251, 109774409-109777830, 109778054-109778597, 109778729- |
| | | | | 109779011, 109779171-109779864, 109779996-109780351, |
| 6 | MYBPHL | NM_001010985 | chr1 | 109835211-109836826, 109836872-109837731, 109837920-109838854, 109838993- |
| | | | | 109839403, 109839565-109839670, 109839812-109840042, 109840240-109840789, 109840880- |
| | | | | 109849467, |
| 6 | GSTM4 | NR_024538 | chr1 | 110199048-110199332, 110199410-110199835, 110199902-110200392, 110200495- |
| | | | | 110201434, 110201532-110201620, 110201733-110203785, |
| 6 | GSTM4 | NM_147148 | chr1 | 110199048-110199332, 110199410-110199835, 110199902-110200210, 110200294- |
| | | | | 110200392, 110200495-110201434, 110201532-110201620, 110201733-110207631, |
| 6 | GSTM4 | NM_000850 | chr1 | 110199048-110199332, 110199410-110199835, 110199902-110200210, 110200294- |
| | | | | 110200392, 110200495-110201434, 110201532-110201620, 110201733-110203785, |
| 6 | GSTM2 | NM_001142368 | chr1 | 110210774-110211045, 110211123-110211545, 110211612-110211910, 110211994- |
| | | | | 110212091, 110212194-110213907, 110214005-110214093, 110214206-110224429, 110224457- |
| | | | | 110225827, |
| 6 | GSTM2 | NM_000848 | chr1 | 110210774-110211045, 110211123-110211545, 110211612-110211910, 110211994- |
| | | | | 110212091, 110212194-110213907, 110214005-110214093, 110214206-110217367, |
| 7 | RPGRIP1 | NM_020366 | chr14 | 21756221-21762834, 21762969-21769123, 21769397-21770645, 21770744-21771488, 21771703- |
| | | | | 21775888, 21775996-21778741, 21778767-21779981, 21780130-21780590, 21780666-21785853, 21786010- |
| | | | | 21788174, 21788337-21789416, 21789562-21790011, 21790164-21792775, 21793230-21793389, 21793543- |
| | | | | 21793988, 21794333-21795780, 21795967-21796581, 21796787-21798406, 21798547-21802762, 21802865- |
| | | | | 21811193, 21811388-21813270, 21813357-21816329, 21816462-21819261, |
| 7 | REM2 | NM_173527 | chr14 | 23352600-23353881, 23354225-23354473, 23354549-23355231, 23355441-23355821, |
| 7 | SLC7A8 | NM_182728 | chr14 | 23596553-23597226, 23597406-23598857, 23599009-23600668, 23600767-23607128, 23607234- |
| | | | | 23608631, 23608757-23609678, 23609834-23612286, 23612414-23623466, |
| 7 | SLC7A8 | NM_012244 | chr14 | 23596553-23597226, 23597406-23598857, 23599009-23600668, 23600767-23607128, 23607234- |
| | | | | 23608631, 23608757-23609678, 23609834-23612286, 23612414-23634492, 23634646-23635543, 23635750- |
| | | | | 23651971, |
| 7 | HOMEZ | NM_020834 | chr14 | 23746397-23755120, |
| 7 | ZFHX2 | NM_033400 | chr14 | 23992151-23992410, 23995889-23996054, 23996183-23996811, 23997014-23998917, 23998978- |
| | | | | 23999327, 23999404-24000688, 24000931-24001774, 24002294-24002492, 24004584-24020550, |
| 7 | DHRS4 | NM_021004 | chr14 | 24423126-24424242, 24424422-24429109, 24429213-24434969, 24435042-24435139, 24435193- |
| | | | | 24435490, 24435627-24436418, 24436476-24437964, |
| 7 | GMPR2 | NM_001002001 | chr14 | 24701995-24702423, 24702547-24702683, 24702805-24704941, 24705027-24705171, 24705347- |
| | | | | 24706275, 24706359-24706449, 24706558-24706724, 24706769-24707450, 24707612-24707793, |
| 7 | GMPR2 | NM_016576 | chr14 | 24702547-24702683, 24702805-24704941, 24705027-24705171, 24705347-24706275, 24706359- |
| | | | | 24706449, 24706558-24706724, 24706769-24707450, 24707612-24707793, |
| 7 | GMPR2 | NM_001002000 | chr14 | 24702054-24702423, 24702547-24702683, 24702805-24704941, 24705027-24705171, 24705347- |
| | | | | 24706275, 24706359-24706449, 24706558-24706724, 24706769-24707450, 24707612-24707793, |
| 7 | GMPR2 | NM_001002002 | chr14 | 24702184-24702423, 24702547-24702683, 24702805-24704941, 24705027-24705171, 24705347- |
| | | | | 24706275, 24706359-24706449, 24706558-24706724, 24706769-24707450, 24707612-24707793, |
| 7 | ADCY4 | NM_139247 | chr14 | 24787775-24787858, 24787985-24788302, 24788419-24788533, 24788643-24788946, 24789095- |
| | | | | 24791270, 24791431-24791828, 24791915-24792108, 24792295-24792548, 24792661-24793266, 24793406- |
| | | | | 24793511, 24793598-24794581, 24794681-24795022, 24795094-24795283, 24795372-24795503, 24795549- |
| | | | | 24798265, 24798441-24798605, 24798740-24799064, 24799225-24799372, 24799502-24800222, 24800336- |
| | | | | 24800412, 24800563-24800992, 24801144-24801726, 24801890-24801995, 24802195-24803698, 24803885- |
| | | | | 24804188, |
| 7 | ADCY4 | NM_001198592 | chr14 | 24787775-24787858, 24787985-24788302, 24788419-24788533, 24788643-24788946, 24789095- |
| | | | | 24791270, 24791431-24791828, 24791915-24792108, 24792295-24792548, 24792661-24793266, 24793406- |
| | | | | 24793511, 24793598-24794581, 24794681-24795022, 24795094-24795283, 24795372-24795503, 24795549- |
| | | | | 24798265, 24798441-24798605, 24798740-24799064, 24799225-24799372, 24799502-24800222, 24800336- |
| | | | | 24800412, 24800563-24800992, 24801144-24801726, 24801890-24801995, 24802195-24803698, 24803889- |
| | | | | 24804188, |
| 7 | ADCY4 | NM_001198568 | chr14 | 24787775-24787858, 24787985-24788302, 24788419-24788533, 24788643-24788946, 24789095- |
| | | | | 24791270, 24791431-24791828, 24791915-24792108, 24792295-24792548, 24792661-24793266, 24793406- |
| | | | | 24793511, 24793598-24794581, 24794681-24795022, 24795094-24795283, 24795372-24795503, 24795549- |
| | | | | 24798265, 24798441-24798605, 24798740-24799064, 24799225-24799372, 24799502-24800222, 24800336- |
| | | | | 24800412, 24800563-24800992, 24801144-24801726, 24801890-24801995, 24802195-24803698, |
| 7 | RIPK3 | NM_006871 | chr14 | 24805602-24806089, 24806152-24806290, 24806667-24806899, 24806969-24807077, 24807247- |
| | | | | 24807453, 24807502-24807626, 24807774-24808219, 24808531-24808661, 24808804-24809011, |
| 7 | CMA1 | NM_001836 | chr14 | 24974866-24975232, 24975489-24975673, 24975811-24976560, 24976713-24977382, |
| 8 | NOSTRIN | NM_001171631 | chr2 | 169643147-169644369, 169644559-169654663, 169654733-169657574, 169657714- |
| | | | | 169658889, 169659184-169668075, 169668163-169681142, 169681228-169684666, 169684731- |
| | | | | 169684928, 169685012-169687980, 169688045-169690735, 169690836-169699500, 169699628- |
| | | | | 169707344, 169707517-169707592, 169707693-169707787, 169707915-169711860, 169711971- |
| | | | | 169713198, 169713289-169716020, 169716148-169717303, 169717420-169718468, 169718560- |
| | | | | 169721342, |
| 8 | NOSTRIN | NM_052946 | chr2 | 169659184-169681142, 169681228-169684666, 169684731-169684928, 169685012- |
| | | | | 169687980, 169688045-169690735, 169690836-169699500, 169699628-169707592, 169707693- |
| | | | | 169707787, 169707915-169711860, 169711971-169713198, 169713289-169716020, 169716148- |
| | | | | 169717303, 169717420-169718468, 169718560-169721342, |
| 8 | NOSTRIN | NM_001039724 | chr2 | 169659184-169668075, 169668163-169681142, 169681228-169684666, 169684731- |
| | | | | 169684928, 169685012-169687980, 169688045-169690735, 169690836-169699500, 169699628- |
| | | | | 169707592, 169707693-169707787, 169707915-169711860, 169711971-169713198, 169713289- |
| | | | | 169716020, 169716148-169717303, 169717420-169718468, 169718560-169721342, |
| 8 | NOSTRIN | NM_001171632 | chr2 | 169659184-169668075, 169668163-169684666, 169684731-169684928, 169685012- |
| | | | | 169687980, 169688045-169690735, 169690836-169699500, 169699628-169707592, 169707693- |
| | | | | 169707787, 169707915-169711860, 169711971-169713198, 169713289-169716020, 169716148- |
| | | | | 169717303, 169717420-169718468, 169718560-169721342, |
| 8 | LRP2 | NM_004525 | chr2 | 169985341-169985521, 169985595-169989082, 169989192-169993900, 169994004- |
| | | | | 169995085, 169995217-169995759, 169995882-169996051, 169996121-169996963, 169997056- |
| | | | | 169999182, 169999304-170002255, 170002434-170003247, 170003470-170007406, 170007537- |
| | | | | 170009307, 170009475-170010968, 170011114-170012782, 170012916-170013879, 170014013- |
| | | | | 170018980, 170019111-170022440, 170022565-170025047, 170025187-170026210, 170026329- |
| | | | | 170027059, 170027178-170028523, 170028645-170029604, 170029735-170030427, 170030675- |
| | | | | 170031701, 170031900-170032919, 170033099-170034311, 170034537-170037956, 170038129- |
| | | | | 170038675, 170038830-170042011, 170042530-170044478, 170044776-170048340, 170048544- |
| | | | | 170050269, 170050403-170053419, 170053541-170055294, 170055422-170058136, 170058381- |
| | | | | 170059264, 170059470-170060490, 170060782-170061987, 170062148-170062531, 170062699- |
| | | | | 170062838, 170063761-170065961, 170066152-170068476, 170068718-170070165, 170070381- |
| | | | | 170072761, 170072941-170076962, 170077074-170081818, 170081964-170082930, 170083099- |
| | | | | 170088222, 170088353-170089919, 170090099-170092348, 170092579-170093611, 170093798- |
| | | | | 170094599, 170094813-170096035, 170096286-170097496, 170097876-170099464, 170099583- |
| | | | | 170099911, 170100033-170101201, 170101443-170103213, 170103497-170103886, 170104026- |
| | | | | 170112614, 170112747-170113632, 170113760-170115533, 170115728-170127412, 170127618- |
| | | | | 170129435, 170129578-170131544, 170131749-170134253, 170134462-170135880, 170136106- |
| | | | | 170136858, 170137030-170139381, 170139512-170145534, 170145656-170147353, 170147508- |
| | | | | 170148761, 170148880-170150656, 170150772-170151108, 170151221-170163789, 170163908- |
| | | | | 170175270, 170175395-170177285, 170177395-170218829, |
| 8 | BBS5 | NM_152384 | chr2 | 170336123-170338759, 170338844-170343577, 170343645-170344314, 170344366- |
| | | | | 170344495, 170344625-170349382, 170349520-170350249, 170350347-170354135, 170354200- |
| | | | | 170355994, 170356131-170359603, 170359689-170360811, 170360837-170360989, |
| 8 | C2orf77 | NM_001085447 | chr2 | 170502693-170505690, 170505857-170506837, 170507032-170510583, 170510725- |
| | | | | 170518788, 170518947-170531084, 170531280-170531375, 170531551-170537515, 170537744- |
| | | | | 170550769, |
| 8 | KLHL23 | NM_144711 | chr2 | 170590598-170591521, 170592738-170597893, 170598048-170605930, |
| 8 | KLHL23 | NM_001199290 | chr2 | 170551133-170551723, 170551758-170553869, 170554042-170591521, 170592738- |
| | | | | 170597893, 170598048-170605930, |
| 8 | CYBRD1 | NM_001127383 | chr2 | 172379249-172409854, 172410011-172411032, |
| 8 | CYBRD1 | NM_024843 | chr2 | 172379249-172398093, 172398304-172409854, 172410011-172411032, |
| 8 | DYNC1I2 | NM_001378 | chr2 | 172544138-172546655, 172546774-172549285, 172549405-172563055, 172563075- |
| | | | | 172563795, 172563888-172569275, 172569337-172571836, 172571954-172582126, 172582224- |
| | | | | 172582427, 172582592-172582702, 172582804-172583299, 172583427-172584329, 172584478- |
| | | | | 172584813, 172584929-172585225, 172585361-172586217, 172586364-172600557, 172600700- |
| | | | | 172602310, 172602438-172604284, |
| 8 | SLC25A12 | NM_003705 | chr2 | 172641986-172644079, 172644172-172644297, 172644458-172647959, 172648100- |
| | | | | 172650135, 172650278-172666114, 172666197-172666732, 172666787-172669847, 172670008- |
| | | | | 172671629, 172671713-172683306, 172683393-172690471, 172690567-172691235, 172691376- |
| | | | | 172693629, 172693778-172700877, 172701019-172712342, 172712460-172725189, 172725334- |
| | | | | 172749713, 172749769-172750711, |
| 8 | PDE11A | NM_001077197 | chr2 | 178494291-178528592, 178528678-178534219, 178534296-178540181, 178540247- |
| | | | | 178545552, 178545632-178562058, 178562161-178565847, 178565940-178576495, 178576607- |
| | | | | 178592384, 178592494-178592752, 178592901-178634049, 178634102-178681554, 178681649- |
| | | | | 178682583, 178682653-178684945, 178685023-178704976, 178705111-178740584, 178740651- |
| | | | | 178762783, 178762926-178769823, 178769915-178879027, 178879188-178969027, 178969204- |
| | | | | 178972979, |
| 8 | PDE11A | NM_016953 | chr2 | 178494291-178528592, 178528678-178534219, 178534296-178540181, 178540247- |
| | | | | 178545552, 178545632-178562058, 178562161-178565847, 178565940-178576495, 178576607- |
| | | | | 178592384, 178592494-178592752, 178592901-178634049, 178634102-178681554, 178681649- |
| | | | | 178682583, 178682653-178684945, 178685023-178704976, 178705111-178740584, 178740651- |
| | | | | 178762783, 178762926-178769823, 178769915-178879027, 178879188-178936251, |
| 8 | PDE11A | NM_001077196 | chr2 | 178494291-178528592, 178528678-178534219, 178534296-178540181, 178540247- |
| | | | | 178545552, 178545632-178562058, 178562161-178565847, 178565940-178576495, 178576607- |
| | | | | 178592384, 178592494-178592752, 178592901-178634049, 178634102-178681554, 178681649- |
| | | | | 178682583, 178682653-178684945, 178685023-178704976, 178705111-178740584, 178740651- |
| | | | | 178753344, |
| 8 | PDE11A | NM_001077358 | chr2 | 178494291-178528592, 178528678-178534219, 178534296-178540181, 178540247- |
| | | | | 178545552, 178545632-178562058, 178562161-178565847, 178565940-178576495, 178576607- |
| | | | | 178592384, 178592494-178592752, 178592901-178634049, 178634102-178681554, 178681649- |
| | | | | 178682583, 178682653-178684945, 178685023-178704976, 178705111-178740584, 178740651- |
| | | | | 178762783, 178762926-178769823, 178769915-178787433, |
| 8 | RBM45 | NM_152945 | chr2 | 178977574-178980987, 178981112-178982780, 178982909-178985012, 178985137- |
| | | | | 178985905, 178986087-178988247, 178988379-178988553, 178988640-178988852, 178989018- |
| | | | | 178990709, 178990912-178994122, |
| 8 | WDR75 | NM_032168 | chr2 | 190306305-190313103, 190313235-190315627, 190315695-190316529, 190316622- |
| | | | | 190320044, 190320171-190322035, 190322108-190323477, 190323599-190324060, 190324151- |
| | | | | 190327208, 190327369-190328418, 190328480-190328570, 190328688-190329803, 190329981- |
| | | | | 190331149, 190331309-190332192, 190332375-190333199, 190333296-190334068, 190334166- |
| | | | | 190334800, 190334972-190335543, 190335605-190338910, 190339059-190339441, 190339535- |
| | | | | 190339937, |
| 8 | SLC40A1 | NM_014585 | chr2 | 190426918-190428308, 190428952-190430078, 190430326-190436439, 190436568- |
| | | | | 190437570, 190437688-190439885, 190440047-190444537, 190444607-190445142, |
| 8 | ANKAR | NM_144708 | chr2 | 190540764-190541180, 190541818-190554251, 190554691-190556979, 190557145- |
| | | | | 190557798, 190557904-190559705, 190559888-190560874, 190561096-190569747, 190569951- |
| | | | | 190571662, 190571873-190575773, 190575880-190584296, 190584540-190585343, 190585514- |
| | | | | 190592580, 190592824-190592991, 190593147-190593384, 190593548-190595219, 190595328- |
| | | | | 190597831, 190597956-190602407, 190602568-190603289, 190603409-190606066, 190606178- |
| | | | | 190607999, 190608201-190609466, 190609515-190611104, |
| 8 | DNAH7 | NM_018897 | chr2 | 196602852-196605488, 196605594-196619059, 196619239-196620855, 196621010- |
| | | | | 196636382, 196636615-196640568, 196640708-196642522, 196642710-196651732, 196651896- |
| | | | | 196659060, 196659263-196661298, 196661462-196664018, 196664197-196671462, 196671557- |
| | | | | 196673404, 196673601-196674467, 196674596-196681350, 196681688-196682418, 196682554- |
| | | | | 196688977, 196689170-196698928, 196699085-196709724, 196709890-196718065, 196718253- |
| | | | | 196720533, 196720716-196722099, 196722448-196723196, 196723529-196726439, 196726671- |
| | | | | 196728871, 196729746-196736972, 196737194-196738290, 196738419-196740397, 196740568- |
| | | | | 196741266, 196741424-196746517, 196746717-196749307, 196749534-196750863, 196750997- |
| | | | | 196752980, 196753159-196753521, 196753706-196756377, 196756529-196759698, 196759949- |
| | | | | 196762389, 196762490-196765004, 196765219-196771381, 196771511-196771629, 196771734- |
| | | | | 196774749, 196774920-196786810, 196786922-196788317, 196788532-196791148, 196791291- |
| | | | | 196799313, 196799514-196801321, 196801517-196821983, 196822172-196824982, 196825670- |
| | | | | 196834670, 196834819-196836964, 196837191-196849354, 196849435-196851788, 196851918- |
| | | | | 196852679, 196852954-196865426, 196865608-196866397, 196866583-196877509, 196877631- |
| | | | | 196883892, 196884020-196889151, 196889229-196891482, 196891665-196892682, 196892772- |
| | | | | 196912074, 196912224-196913018, 196913129-196915909, 196915974-196922776, 196922841- |
| | | | | 196933419, |
| 9 | PHLDB1 | NM_001144759 | chr11 | 118478415-118484529, 118484612-118485272, 118485398-118486754, 118486927- |
| | | | | 118495648, 118495776-118498019, 118499367-118501922, 118502197-118502628, 118502771- |
| | | | | 118502874, 118503014-118506109, 118506267-118509607, 118509720-118509878, 118509970- |
| | | | | 118514516, 118514659-118514788, 118514893-118516072, 118516369-118516451, 118516544- |
| | | | | 118518644, 118518793-118520780, 118520899-118521148, 118521253-118526313, 118526401- |
| | | | | 118527391, |
| 9 | PHLDB1 | NM_001144758 | chr11 | 118478415-118484529, 118484612-118485272, 118485398-118486754, 118486927- |
| | | | | 118495648, 118495776-118498019, 118499367-118501922, 118502197-118502628, 118502771- |
| | | | | 118502874, 118503014-118506109, 118506267-118509607, 118509720-118509878, 118509970- |
| | | | | 118512970, 118513113-118514516, 118514659-118514788, 118514893-118516072, 118516369- |
| | | | | 118516451, 118516544-118518644, 118518793-118520780, 118520899-118521148, 118521253- |
| | | | | 118526313, 118526401-118526568, 118526603-118527391, |
| 9 | PHLDB1 | NM_015157 | chr11 | 118477388-118484007, 118484166-118484529, 118484612-118485272, 118485398- |
| | | | | 118486754, 118486927-118495648, 118495776-118498019, 118499367-118501922, 118502197- |
| | | | | 118502628, 118502771-118502874, 118503014-118506109, 118506267-118509607, 118509720- |
| | | | | 118509878, 118509970-118512970, 118513113-118514516, 118514659-118514788, 118514893- |
| | | | | 118516072, 118516369-118516451, 118516544-118518644, 118518793-118520780, 118520899- |
| | | | | 118521148, 118521253-118526313, 118526401-118526568, 118526603-118527391, |
| 9 | HINFP | NM_198971 | chr11 | 118992350-118997643, 118997836-119001433, 119001665-119002243, 119002357- |
| | | | | 119002538, 119002693-119003204, 119003284-119003369, 119003492-119003572, 119003713- |
| | | | | 119003803, 119003930-119004792, |
| 9 | HINFP | NM_015517 | chr11 | 118992350-118994276, 118994395-118997643, 118997836-119001433, 119001665- |
| | | | | 119002243, 119002357-119002538, 119002693-119003204, 119003284-119003369, 119003492- |
| | | | | 119003572, 119003713-119003803, 119003930-119004792, |
| 9 | PDZD3 | NM_001168468 | chr11 | 119056220-119056632, 119056662-119056799, 119056875-119057186, 119057400- |
| | | | | 119057977, 119058169-119058271, 119058410-119058647, 119058854-119059062, 119059261- |
| | | | | 119059347, 119059577-119059712, 119059915-119060170, |
| 9 | PDZD3 | NR_033122 | chr11 | 119056220-119056632, 119056662-119056799, 119056951-119057186, 119057400- |
| | | | | 119057977, 119058169-119058271, 119058410-119058647, 119058854-119059062, 119059261- |
| | | | | 119059347, 119059577-119059712, 119059915-119060170, |
| 9 | PDZD3 | NM_024791 | chr11 | 119056220-119056632, 119056662-119056799, 119056875-119057186, 119057400- |
| | | | | 119057977, 119058169-119058271, 119058368-119058647, 119058854-119059062, 119059261- |
| | | | | 119059347, 119059577-119059712, 119059915-119060170, |
| 9 | CCDC153 | NM_001145018 | chr11 | 119061162-119061417, 119061550-119063859, 119063954-119064474, 119064596- |
| | | | | 119065631, 119065705-119066132, 119066210-119066507, |
| 9 | GRIK4 | NM_014619 | chr11 | 120531110-120673400, 120673567-120686085, 120686185-120690462, 120690630- |
| | | | | 120702559, 120702740-120707498, 120707554-120732666, 120732830-120744773, 120744928- |
| | | | | 120745846, 120745953-120769239, 120769349-120775997, 120776203-120811054, 120811170- |
| | | | | 120823562, 120823674-120827487, 120827663-120831616, 120831784-120833163, 120833391- |
| | | | | 120837902, 120838033-120852813, 120852934-120856611, |
| 9 | TBCEL | NM_152715 | chr11 | 120895001-120916381, 120916533-120918235, 120918377-120924258, 120924442- |
| | | | | 120925759, 120926018-120929052, 120929181-120930676, 120930795-120957485, |
| 9 | TBCEL | NM_001130047 | chr11 | 120894864-120916381, 120916533-120918235, 120918377-120924258, 120924442- |
| | | | | 120925759, 120926018-120929052, 120929181-120930676, 120930795-120957485, |
| 9 | TECTA | NM_005422 | chr11 | 120973439-120976538, 120976674-120979918, 120980208-120983779, 120983919- |
| | | | | 120984260, 120984428-120989013, 120989428-120996009, 120996582-120998459, 120999054- |
| | | | | 121000345, 121000921-121008128, 121008732-121016262, 121016826-121023588, 121023790- |
| | | | | 121028548, 121028934-121030842, 121031131-121032782, 121033080-121035980, 121036093- |
| | | | | 121037285, 121037490-121038761, 121038927-121039384, 121039635-121058539, 121058704- |
| | | | | 121059787, 121059877-121060471, 121060590-121061413, |
| 9 | VWA5A | NM_014622 | chr11 | 123986190-123988202, 123988262-123988378, 123988583-123988894, 123989119- |
| | | | | 123989238, 123989416-123989680, 123989797-123993665, 123993837-123994071, 123994162- |
| | | | | 123994365, 123994512-123994942, 123995024-124005625, 124005742-124006834, 124007001- |
| | | | | 124007279, 124007382-124007720, 124007976-124012303, 124012449-124013147, 124013280- |
| | | | | 124015942, 124016071-124016571, |
| 9 | VWA5A | NM_001130142 | chr11 | 123986190-123987271, 123987388-123988202, 123988262-123988378, 123988583- |
| | | | | 123988894, 123989119-123989238, 123989416-123989680, 123989797-123993665, 123993837- |
| | | | | 123994071, 123994162-123994365, 123994512-123994942, 123995024-124005625, 124005742- |
| | | | | 124006834, 124007001-124007279, 124007382-124007720, 124007976-124012303, 124012449- |
| | | | | 124013147, 124013280-124015942, 124016071-124016571, |
| 9 | VWA5A | NM_198315 | chr11 | 123986190-123988202, 123988262-123988378, 123988583-123988894, 123989119- |
| | | | | 123989238, 123989416-123989680, 123989797-123993665, 123993837-123994071, 123994162- |
| | | | | 123994365, 123994512-123994942, |
| 9 | SPA17 | NM_017425 | chr11 | 124543849-124545132, 124545315-124551283, 124551356-124561590, 124561679-124564197, |
| 9 | KCNJ5 | NM_000890 | chr11 | 128761617-128781157, 128782106-128786302, |
| 9 | ADAMTS8 | NM_007037 | chr11 | 130276024-130278357, 130278535-130278661, 130278836-130281310, 130281496- |
| | | | | 130284424, 130284728-130286056, 130286226-130286833, 130286971-130288946, 130289188- |
| | | | | 130297460, |
| 10 | ISOC2 | NM_001136202 | chr19 | 55964756-55966354, 55966474-55966625, 55966698-55967714, 55967857-55972877, |
| 10 | ISOC2 | NM_001136201 | chr19 | 55964756-55966354, 55966474-55966625, 55966698-55967001, 55967213-55967714, 55967857-55972877, |
| 10 | ISOC2 | NM_024710 | chr19 | 55964756-55966354, 55966474-55966625, 55966746-55967001, 55967213-55967714, 55967857-55972877, |
| 10 | NLRP8 | NM_176811 | chr19 | 56459636-56463902, 56463979-56465865, 56467467-56473431, 56473604-56477577, 56477747- |
| | | | | 56481908, 56482063-56485016, 56485189-56487497, 56487670-56490758, 56490931-56499178, |
| 10 | LOC386758 | NR_037159 | chr19 | 56905423-56905584, 56905811-56909248, 56909418-56909644, |
| 10 | LOC386758 | NR_037161 | chr19 | 56905811-56909248, 56909418-56909644, 56909753-56910261, |
| 10 | LOC386758 | NR_037160 | chr19 | 56905423-56909248, |
| 10 | ZFP28 | NM_020828 | chr19 | 57050596-57050992, 57051086-57058875, 57059004-57059174, 57059272-57060325, 57060491- |
| | | | | 57061434, 57061551-57061877, 57061975-57065051, |
| 10 | ZNF71 | NM_021216 | chr19 | 57106797-57112873, 57112960-57132634, |
| 10 | ZNF835 | NM_001005850 | chr19 | 57176614-57184226, |
| 10 | ZNF132 | NM_003433 | chr19 | 58946579-58948412, 58948583-58951124, |
| 10 | CHMP2A | NM_014453 | chr19 | 59063144-59063271, 59063335-59063420, 59063553-59063624, 59063806-59065410, 59065604-59066353, |
| 10 | CHMP2A | NM_198426 | chr19 | 59063144-59063271, 59063335-59063420, 59063553-59063624, 59063806-59065410, 59065604-59066230, |
| 11 | POU4F3 | NM_002700 | chr5 | 145718796-145719109, |
| 11 | TCERG1 | NM_001040006 | chr5 | 145826972-145834617, 145834845-145836744, 145836899-145838445, 145838901- |
| | | | | 145843112, 145843357-145849105, 145849308-145850197, 145850312-145851049, 145851140- |
| | | | | 145858054, 145858217-145859416, 145859475-145859589, 145859658-145862153, 145862291- |
| | | | | 145863101, 145863193-145872481, 145872602-145878097, 145878250-145882936, 145883103- |
| | | | | 145883385, 145883570-145886589, 145886771-145887434, 145887520-145888706, 145888809- |
| | | | | 145890002, |
| 11 | TCERG1 | NM_006706 | chr5 | 145826972-145834617, 145834845-145836744, 145836899-145838445, 145838901- |
| | | | | 145843112, 145843357-145847902, 145847967-145849105, 145849308-145850197, 145850312- |
| | | | | 145851049, 145851140-145858054, 145858217-145859416, 145859475-145859589, 145859658- |
| | | | | 145862153, 145862291-145863101, 145863193-145872481, 145872602-145878097, 145878250- |
| | | | | 145882936, 145883103-145883385, 145883570-145886589, 145886771-145887434, 145887520- |
| | | | | 145888706, 145888809-145890002, |
| 11 | SPINK5 | NM_006846 | chr5 | 147443663-147444908, 147444936-147449884, 147450014-147451709, 147451784- |
| | | | | 147465966, 147466096-147468103, 147468169-147469055, 147469185-147470726, 147470792- |
| | | | | 147473915, 147474045-147475379, 147475469-147477428, 147477558-147478795, 147478879- |
| | | | | 147480015, 147480145-147480916, 147481000-147481342, 147481472-147484513, 147484564- |
| | | | | 147486598, 147486728-147488314, 147488401-147491329, 147491459-147492429, 147492498- |
| | | | | 147493923, 147494053-147495931, 147496030-147497998, 147498128-147498547, 147498622- |
| | | | | 147499570, 147499700-147499856, 147499955-147503394, 147503524-147504326, 147504401- |
| | | | | 147505284, 147505414-147506544, 147506643-147510820, 147510953-147513358, 147513451- |
| | | | | 147516544, |
| 11 | SPINK5 | NM_001127699 | chr5 | 147443663-147444908, 147444936-147449884, 147450014-147451709, 147451784- |
| | | | | 147465966, 147466096-147468103, 147468169-147469055, 147469185-147470726, 147470792- |
| | | | | 147473915, 147474045-147475379, 147475469-147477428, 147477558-147478795, 147478879- |
| | | | | 147480015, 147480145-147480916, 147481000-147481342, 147481472-147484513, 147484564- |
| | | | | 147486598, 147486728-147488314, 147488401-147491329, 147491459-147492429, 147492498- |
| | | | | 147493923, 147494053-147495931, 147496030-147497998, 147498128-147498547, 147498622- |
| | | | | 147499570, 147499700-147499856, 147499955-147503394, 147503524-147504326, |
| 11 | SPINK5 | NM_001127698 | chr5 | 147443663-147444908, 147444936-147449884, 147450014-147451709, 147451784- |
| | | | | 147465966, 147466096-147468103, 147468169-147469055, 147469185-147470726, 147470792- |
| | | | | 147473915, 147474045-147475379, 147475469-147477428, 147477558-147478795, 147478879- |
| | | | | 147480015, 147480145-147480916, 147481000-147481342, 147481472-147484513, 147484564- |
| | | | | 147486598, 147486728-147488314, 147488401-147491329, 147491459-147492429, 147492498- |
| | | | | 147493923, 147494053-147495931, 147496030-147497998, 147498128-147498547, 147498622- |
| | | | | 147499570, 147499700-147499856, 147499955-147503394, 147503524-147504326, 147504401- |
| | | | | 147505085, 147505177-147505284, 147505414-147506544, 147506643-147510820, 147510953- |
| | | | | 147513358, 147513451-147516544, |
| 11 | GLRA1 | NM_000171 | chr5 | 151202525-151208480, 151208629-151230949, 151231166-151234599, 151234739- |
| | | | | 151235860, 151235945-151239344, 151239570-151266280, 151266350-151271870, 151272000- |
| | | | | 151304053, |
| 11 | GLRA1 | NM_001146040 | chr5 | 151202549-151208480, 151208629-151230949, 151231166-151234599, 151234739- |
| | | | | 151235860, 151235945-151239344, 151239570-151266280, 151266350-151271870, 151272000- |
| | | | | 151304053, |
| 11 | CLINT1 | NM_014666 | chr5 | 157215001-157216369, 157216522-157218709, 157219004-157221904, 157221981- |
| | | | | 157230656, 157230728-157232872, 157233121-157236634, 157236814-157240069, 157240236- |
| | | | | 157241191, 157241302-157243740, 157243839-157244449, 157244556-157285936, |
| 11 | CLINT1 | NM_001195556 | chr5 | 157215001-157216369, 157216576-157218709, 157219004-157221904, 157221981- |
| | | | | 157230656, 157230728-157232872, 157233121-157236634, 157236814-157240069, 157240236- |
| | | | | 157241191, 157241302-157243740, 157243839-157244449, 157244556-157285959, |
| 11 | CLINT1 | NM_001195555 | chr5 | 157215001-157216369, 157216576-157218709, 157219004-157221904, 157221981- |
| | | | | 157230656, 157230728-157232872, 157233121-157236634, 157236814-157240069, 157240236- |
| | | | | 157241191, 157241302-157243740, 157243839-157244449, 157244556-157285936, |
| 11 | RNF145 | NM_001199383 | chr5 | 158586044-158588272, 158588631-158589938, 158590088-158595879, 158596064- |
| | | | | 158596685, 158596828-158600989, 158601167-158603638, 158603876-158608966, 158609060- |
| | | | | 158621722, 158621833-158630440, 158630665-158636237, |
| 11 | RNF145 | NM_001199382 | chr5 | 158586044-158588272, 158588631-158589938, 158590088-158595879, 158596064- |
| | | | | 158596685, 158596828-158600989, 158601167-158603638, 158603876-158608966, 158609060- |
| | | | | 158621722, 158621833-158630440, 158630665-158634481, |
| 11 | RNF145 | NM_144726 | chr5 | 158586044-158588272, 158588631-158589938, 158590088-158595879, 158596064- |
| | | | | 158596685, 158596828-158600989, 158601167-158603638, 158603876-158608966, 158609060- |
| | | | | 158621722, 158621833-158630440, 158630665-158634761, |
| 11 | RNF145 | NM_001199381 | chr5 | 158586044-158588272, 158588631-158589938, 158590088-158595879, 158596064- |
| | | | | 158596685, 158596828-158600989, 158601167-158603638, 158603876-158608966, 158609060- |
| | | | | 158621722, 158621833-158630440, 158630665-158634891, |
| 11 | RNF145 | NM_001199380 | chr5 | 158586044-158588272, 158588631-158589938, 158590088-158595879, 158596064- |
| | | | | 158596685, 158596828-158600989, 158601167-158603638, 158603876-158608966, 158609060- |
| | | | | 158621722, 158621833-158630440, 158630665-158636859, |
| 11 | UBLCP1 | NM_145049 | chr5 | 158690369-158695876, 158696078-158696914, 158697008-158697366, 158697454- |
| | | | | 158697534, 158697652-158699029, 158699130-158701591, 158701631-158702154, 158702255- |
| | | | | 158705244, 158705363-158710218, 158710348-158711910, |
| 11 | LOC285629 | NR_027111 | chr5 | 160360815-160362995, 160363117-160365459, |
| 11 | GABRG2 | NM_198904 | chr5 | 161495113-161520832, 161520986-161522499, 161522569-161524642, 161524865- |
| | | | | 161528239, 161528324-161530893, 161531033-161569168, 161569323-161576112, 161576320- |
| | | | | 161578734, 161578760-161580097, |
| 11 | GABRG2 | NM_198903 | chr5 | 161495113-161520832, 161520986-161522499, 161522569-161524642, 161524865- |
| | | | | 161528239, 161528324-161529558, 161529680-161530893, 161531033-161569168, 161569323- |
| | | | | 161576112, 161576320-161578734, 161578760-161580097, |
| 11 | GABRG2 | NM_000816 | chr5 | 161495113-161520832, 161520986-161522499, 161522569-161524642, 161524865- |
| | | | | 161528239, 161528324-161530893, 161531033-161569168, 161569323-161576112, 161576320- |
| | | | | 161580097, |
| 11 | HMMR | NM_001142557 | chr5 | 162887745-162896645, 162896836-162898189, 162898278-162898367, 162898470- |
| | | | | 162900183, 162900260-162900383, 162900564-162901061, 162901212-162902462, 162902679- |
| | | | | 162905688, 162905807-162909646, 162909795-162910024, 162910179-162910272, 162910374- |
| | | | | 162911073, 162911252-162917394, 162917559-162918116, |
| 11 | HMMR | NM_012485 | chr5 | 162887745-162890860, 162890961-162891727, 162891809-162896645, 162896836- |
| | | | | 162898189, 162898278-162898367, 162898470-162900183, 162900260-162900383, 162900564- |
| | | | | 162901061, 162901212-162902462, 162902679-162905688, 162905807-162909646, 162909795- |
| | | | | 162910024, 162910179-162910272, 162910374-162911073, 162911252-162917394, 162917559- |
| | | | | 162918116, |
| 11 | HMMR | NM_012484 | chr5 | 162887745-162890860, 162890961-162891727, 162891809-162894708, 162894755- |
| | | | | 162896645, 162896836-162898189, 162898278-162898367, 162898470-162900183, 162900260- |
| | | | | 162900383, 162900564-162901061, 162901212-162902462, 162902679-162905688, 162905807- |
| | | | | 162909646, 162909795-162910024, 162910179-162910272, 162910374-162911073, 162911252- |
| | | | | 162917394, 162917559-162918116, |
| 11 | HMMR | NM_001142556 | chr5 | 162887745-162890860, 162890961-162891727, 162891809-162894705, 162894755- |
| | | | | 162896645, 162896836-162898189, 162898278-162898367, 162898470-162900183, 162900260- |
| | | | | 162900383, 162900564-162901061, 162901212-162902462, 162902679-162905688, 162905807- |
| | | | | 162909646, 162909795-162910024, 162910179-162910272, 162910374-162911073, 162911252- |
| | | | | 162917394, 162917559-162918116, |
| 11 | TLX3 | NM_021025 | chr5 | 170736791-170737152, 170737398-170738391, |
| 11 | NPM1 | NM_199185 | chr5 | 170815011-170817053, 170817135-170818307, 170818429-170818708, 170818804- |
| | | | | 170819712, 170819821-170819916, 170819983-170827155, 170827215-170832304, 170832408- |
| | | | | 170834702, 170834779-170837529, |
| 11 | NPM1 | NM_002520 | chr5 | 170815011-170817053, 170817135-170818307, 170818429-170818708, 170818804- |
| | | | | 170819712, 170819821-170819916, 170819983-170827155, 170827215-170827841, 170827930- |
| | | | | 170832304, 170832408-170834702, 170834779-170837529, |
| 11 | NPM1 | NM_001037738 | chr5 | 170815011-170817053, 170817135-170818307, 170818429-170818708, 170818804- |
| | | | | 170819712, 170819821-170819916, 170819983-170827155, 170827215-170827841, 170827930- |
| | | | | 170832304, 170832408-170833399, |
| 12 | LMAN1L | NM_021819 | chr15 | 75105371-75108496, 75108653-75108766, 75108876-75108971, 75109032-75111057, 75111159- |
| | | | | 75111491, 75111614-75112383, 75112441-75112974, 75113109-75113414, 75113568-75114168, 75114242- |
| | | | | 75114981, 75115051-75115898, 75116024-75116690, 75116820-75117815, |
| 12 | CPLX3 | NM_001030005 | chr15 | 75119209-75120373, 75120463-75122469, |
| 12 | COX5A | NM_004255 | chr15 | 75212784-75215987, 75216112-75219105, 75219229-75221455, 75221574-75230254, |
| 12 | FBXO22 | NM_012170 | chr15 | 76196445-76196830, 76196971-76205542, 76205632-76206449, 76206547-76209569, 76209736-76222223, |
| 12 | FBXO22 | NR_037623 | chr15 | 76196445-76205542, 76205632-76206449, 76206547-76209569, 76209736-76222223, 76222391-76225024, |
| 12 | FBXO22 | NM_147188 | chr15 | 76196445-76196830, 76196971-76205542, 76205632-76206449, 76206547-76209569, 76209736- |
| | | | | 76222223, 76222391-76225024, |
| 12 | SCAPER | NM_020843 | chr15 | 76641078-76643576, 76643630-76646288, 76646482-76668501, 76668653-76673717, 76673957- |
| | | | | 76696863, 76697021-76726417, 76726652-76763551, 76763677-76797198, 76797316-76866497, 76866626- |
| | | | | 76914103, 76914171-76957992, 76958131-76994097, 76994207-76995189, 76995344-76998242, 76998326- |
| | | | | 77020934, 77021079-77025568, 77025726-77046147, 77046290-77057300, 77057414-77057676, 77057796- |
| | | | | 77057902, 77057980-77059257, 77059430-77064081, 77064296-77067194, 77067459-77087619, 77087782- |
| | | | | 77092587, 77092706-77096872, 77096975-77134073, 77134273-77150148, 77150221-77154755, 77154875- |
| | | | | 77176151, |
| 12 | SCAPER | NM_001145923 | chr15 | 76641078-76643576, 76643630-76646288, 76646482-76668501, 76668653-76673717, 76673957- |
| | | | | 76696863, 76697021-76726417, 76726652-76763551, 76763677-76797198, 76797316-76866497, 76866626- |
| | | | | 76914103, 76914171-76957992, 76958131-76994097, 76994207-76995189, 76995344-76998242, 76998326- |
| | | | | 77020934, 77021079-77025568, 77025726-77046147, 77046290-77057300, 77057414-77057676, 77057796- |
| | | | | 77057902, 77057980-77059257, 77059430-77064081, 77064296-77067194, 77067459-77085572, 77085733- |
| | | | | 77087619, 77087782-77092587, 77092706-77096872, 77096975-77134073, 77134273-77148149, 77148218- |
| | | | | 77150148, 77150221-77154184, |
| 12 | LINGO1 | NM_032808 | chr15 | 77908243-77924650, |
| 12 | CRABP1 | NM_004378 | chr15 | 78632841-78633383, 78633564-78635839, 78635955-78640267, |
| 12 | RASGRF1 | NM_153815 | chr15 | 79254579-79264206, 79264277-79265643, 79265763-79272960, 79273042-79277347, 79277547- |
| | | | | 79282529, 79282616-79284031, 79284137-79288080, 79288143-79290436, 79290551-79291059, 79291168- |
| | | | | 79292083, 79292256-79294002, 79294130-79296142, 79296518-79297057, |
| 12 | RASGRF1 | NM_002891 | chr15 | 79254579-79264206, 79264277-79265643, 79265763-79272960, 79273042-79277347, 79277547- |
| | | | | 79282529, 79282616-79284031, 79284137-79288080, 79288143-79290436, 79290551-79291059, 79291168- |
| | | | | 79292083, 79292256-79294002, 79294130-79296142, 79296518-79298517, 79298777-79304829, 79304870- |
| | | | | 79307667, 79307752-79310110, 79310249-79312381, 79312447-79317654, 79317817-79320081, 79320202- |
| | | | | 79323740, 79323852-79324463, 79324659-79327471, 79327553-79339086, 79339342-79341836, 79341931- |
| | | | | 79350674, 79350824-79356760, 79356869-79382563, |
| 12 | RASGRF1 | NM_001145648 | chr15 | 79254579-79264206, 79264277-79265643, 79265763-79272960, 79273042-79277347, 79277547- |
| | | | | 79282529, 79282616-79284031, 79284137-79288080, 79288143-79290436, 79290551-79291059, 79291168- |
| | | | | 79292083, 79292256-79294002, 79294130-79296142, 79296518-79298526, 79298777-79307667, 79307752- |
| | | | | 79310110, 79310249-79312381, 79312447-79317654, 79317817-79320081, 79320202-79323740, 79323852- |
| | | | | 79324463, 79324659-79327471, 79327553-79339086, 79339342-79341836, 79341931-79350674, 79350824- |
| | | | | 79356760, 79356869-79382563, |
| 12 | KIAA1024 | NM_015206 | chr15 | 79724883-79748438, 79750788-79755407, 79755664-79760527, |
| 13 | SECISBP2 | NM_024077 | chr9 | 91933528-91934565, 91934713-91940340, 91940592-91940810, 91940954-91943573, 91943802- |
| | | | | 91947821, 91947902-91949435, 91949646-91953371, 91953496-91954777, 91954869-91956260, 91956395- |
| | | | | 91961795, 91961964-91962992, 91963130-91964689, 91964845-91965545, 91965768-91972324, 91972481- |
| | | | | 91972912, 91973107-91973634, |
| 13 | NOL8 | NM_017948 | chr9 | 95060169-95060537, 95060619-95061164, 95061237-95062170, 95062299-95063831, 95064105- |
| | | | | 95068033, 95068113-95069166, 95069307-95072451, 95072544-95072833, 95072958-95073426, 95073542- |
| | | | | 95076547, 95078419-95080896, 95080969-95081501, 95081639-95083948, 95084029-95085721, 95085786- |
| | | | | 95086303, 95086492-95087586, |
| 13 | NOL8 | NR_024020 | chr9 | 95060169-95060537, 95060619-95061164, 95061237-95062170, 95062299-95063831, 95064105- |
| | | | | 95068033, 95068113-95069166, 95069307-95072451, 95072544-95072833, 95072958-95073426, 95073542- |
| | | | | 95076547, 95078419-95080896, 95080969-95081501, 95081639-95083948, 95084029-95085516, 95085634- |
| | | | | 95085721, 95085786-95086303, 95086492-95087598, |
| 13 | CENPP | NM_001012267 | chr9 | 95088388-95094450, 95094634-95099821, 95099912-95107979, 95108070-95142043, 95142142- |
| | | | | 95373594, 95373676-95374803, 95374897-95375299, |
| 13 | BICD2 | NM_001003800 | chr9 | 95477746-95480077, 95480231-95480819, 95481865-95482580, 95483038-95484936, 95485091- |
| | | | | 95491304, 95491519-95526785, |
| 13 | BICD2 | NM_015250 | chr9 | 95475744-95477533, 95477746-95480077, 95480231-95480819, 95481865-95482580, 95483038- |
| | | | | 95484936, 95485091-95491304, 95491519-95526785, |
| 13 | ANKRD19 | NR_026868 | chr9 | 95572153-95575876, 95575993-95576154, 95576330-95580598, 95580707-95581522, 95581662- |
| | | | | 95584490, 95584560-95588805, 95588861-95591218, 95591446-95599057, 95600023-95600537, |
| 13 | ZNF484 | NM_001007101 | chr9 | 95610834-95618091, 95618186-95618472, 95618601-95640170, |
| 13 | ZNF484 | NM_031486 | chr9 | 95610834-95618091, 95618186-95618472, 95618601-95637295, 95637342-95640170, |
| 13 | FGD3 | NM_001083536 | chr9 | 95710012-95737518, 95737688-95738488, 95738992-95765205, 95765297-95766281, 95766420- |
| | | | | 95768304, 95768463-95772526, 95772667-95773494, 95773555-95776132, 95776281-95778017, 95778112- |
| | | | | 95780416, 95780498-95782111, 95782144-95782597, 95782707-95784607, 95784672-95792154, 95792279- |
| | | | | 95795049, 95795156-95796821, 95796964-95797618, |
| 13 | FGD3 | NM_033086 | chr9 | 95737029-95737518, 95737688-95738488, 95738992-95765205, 95765297-95766281, 95766420- |
| | | | | 95768304, 95768463-95772526, 95772667-95773494, 95773555-95776132, 95776281-95778017, 95778112- |
| | | | | 95780416, 95780498-95782111, 95782144-95782597, 95782707-95784607, 95784672-95792154, 95792279- |
| | | | | 95795049, 95795156-95796821, 95796964-95797618, |
| 13 | SUSD3 | NM_145006 | chr9 | 95821113-95838064, 95838255-95840126, 95840276-95841751, 95841885-95846817, |
| 13 | C9orf89 | NM_032310 | chr9 | 95858635-95869954, 95870099-95872848, 95873004-95874159, 95874221-95874498, 95874572-95875272, |
| 13 | NINJ1 | NM_004148 | chr9 | 95884513-95887179, 95887345-95888690, 95888921-95896423, |
| 13 | FAM120A | NM_014612 | chr9 | 96214672-96233421, 96233670-96238536, 96238621-96259751, 96259882-96261070, 96261169- |
| | | | | 96277947, 96278050-96278263, 96278552-96289435, 96289525-96291633, 96291863-96294435, 96294612- |
| | | | | 96305502, 96305754-96312857, 96312974-96318662, 96318874-96320107, 96320293-96320861, 96321001- |
| | | | | 96323389, 96323533-96324488, 96324587-96326509, |
| 13 | PHF2 | NM_005392 | chr9 | 96339154-96392250, 96392338-96398691, 96398808-96407909, 96408072-96411351, 96411495- |
| | | | | 96415459, 96415648-96416693, 96416858-96418214, 96418304-96418769, 96418878-96420425, 96420519- |
| | | | | 96421791, 96421883-96422472, 96422843-96425187, 96425309-96425797, 96425918-96427966, 96428147- |
| | | | | 96428270, 96428343-96429360, 96429523-96435865, 96436145-96437207, 96437305-96437960, 96438072- |
| | | | | 96438874, 96439246-96439868, |
| 13 | FBP1 | NM_000507 | chr9 | 97365855-97367737, 97367859-97369095, 97369235-97372201, 97372344-97380048, 97380143- |
| | | | | 97382609, 97382774-97401421, |
| 13 | FBP1 | NM_001127628 | chr9 | 97365855-97367737, 97367859-97369095, 97369235-97372201, 97372344-97380048, 97380143- |
| | | | | 97382609, 97382774-97401421, 97401617-97402324, |
| 13 | C9orf130 | NR_023389 | chr9 | 98632724-98636789, 98636905-98637260, |
| 13 | C9orf130 | NR_023390 | chr9 | 98576205-98612507, 98612575-98638145, |
| 13 | C9orf102 | NM_001010895 | chr9 | 98638367-98643149, 98643576-98660129, 98660254-98669358, 98669554-98677948, 98678112- |
| | | | | 98678507, 98678717-98683455, 98683598-98684585, 98684701-98685540, 98685662-98690359, 98690433- |
| | | | | 98690999, 98691147-98703734, 98703832-98718194, 98718296-98728842, |
| 13 | SLC35D2 | NM_007001 | chr9 | 99083624-99084278, 99084363-99086371, 99086452-99098997, 99099067-99106184, 99106279- |
| | | | | 99107579, 99107684-99113383, 99113454-99114318, 99114392-99122434, 99122504-99126744, 99126833- |
| | | | | 99130552, 99130588-99145757, |
| 13 | ZNF510 | NM_014930 | chr9 | 99522760-99525398, 99525496-99525786, 99525915-99537068, 99537129-99538280, 99538528-99540314, |
| 13 | ZNF782 | NM_001001662 | chr9 | 99582061-99589360, 99589464-99607170, 99607299-99614227, 99614288-99615133, 99615352-99616368, |
| 13 | LOC57653 | NR_036527 | chr9 | 100000850-100008376, 100008469-100013072, 100013126-100013821, 100013840- |
| | | | | 100038849, 100038980-100039836, 100039909-100041642, 100041756-100043796, 100043952- |
| | | | | 100049940, 100050018-100052864, 100052948-100053134, 100053185-100053651, 100053770- |
| | | | | 100056227, 100056393-100057125, 100057274-100070016, 100070089-100070263, 100070415- |
| | | | | 100071694, 100071887-100074393, 100074484-100075516, 100075628-100076673, 100076804- |
| | | | | 100076898, 100077011-100077131, 100077229-100079345, 100079511-100080743, 100080865- |
| | | | | 100082406, 100082488-100085113, 100085204-100086061, 100086186-100087299, 100087475- |
| | | | | 100088834, 100088952-100090300, 100090429-100092434, 100092476-100092602, 100093050- |
| | | | | 100105620, 100105846-100116849, 100116996-100117172, 100117291-100119907, 100120008- |
| | | | | 100122262, 100122384-100124006, 100124181-100124476, 100124534-100124643, 100124724- |
| | | | | 100126298, 100126411-100127953, 100128022-100128839, 100128983-100132202, 100132365- |
| | | | | 100132921, 100133061-100133875, 100133989-100136823, 100136931-100137734, 100137872- |
| | | | | 100139043, |
| 13 | LOC57653 | NR_036529 | chr9 | 100000850-100008376, 100008469-100013072, 100013126-100013821, 100013840- |
| | | | | 100038849, 100038980-100039836, 100039909-100041642, 100041756-100043796, 100043952- |
| | | | | 100049940, 100050018-100052864, 100052948-100053134, 100053185-100053651, 100053770- |
| | | | | 100070016, 100070089-100070263, 100070415-100071694, 100071887-100074393, 100074484- |
| | | | | 100075516, 100075628-100076898, 100077011-100077131, 100077229-100079345, 100079511- |
| | | | | 100080743, 100080865-100082406, 100082488-100085113, 100085204-100086061, 100086186- |
| | | | | 100087299, 100087475-100088834, 100088952-100090300, 100090429-100092434, 100092476- |
| | | | | 100092602, 100093050-100105620, 100105846-100116849, 100116996-100117172, 100117291- |
| | | | | 100119907, 100120008-100122262, 100122384-100124006, 100124181-100124476, 100124534- |
| | | | | 100124643, 100124724-100127953, 100128022-100128839, 100128983-100132202, 100132365- |
| | | | | 100132921, 100133061-100133875, 100133989-100136823, 100136931-100137734, 100137872- |
| | | | | 100139043, |
| 13 | LOC57653 | NR_036528 | chr9 | 100000850-100008376, 100008469-100013072, 100013126-100013821, 100013840- |
| | | | | 100038849, 100038980-100039836, 100039909-100041642, 100041756-100043796, 100043952- |
| | | | | 100049940, 100050018-100052864, 100052948-100053134, 100053185-100053651, 100053770- |
| | | | | 100056227, 100056393-100057125, 100057274-100070016, 100070089-100070263, 100070415- |
| | | | | 100071694, 100071887-100074393, 100074484-100075516, 100075628-100076673, 100076804- |
| | | | | 100076898, 100077011-100077131, 100077229-100079345, 100079511-100080743, 100080865- |
| | | | | 100082406, 100082488-100085113, 100085204-100086061, 100086186-100087299, 100087475- |
| | | | | 100088834, 100088952-100090300, 100090429-100092434, 100092476-100092602, 100093050- |
| | | | | 100105620, 100105846-100109594, 100109772-100111391, 100111574-100112689, 100112838- |
| | | | | 100116849, 100116996-100117172, 100117291-100119907, 100120008-100122262, 100122384- |
| | | | | 100124006, 100124181-100124476, 100124724-100126331, 100126411-100127953, 100128022- |
| | | | | 100128839, 100128983-100132202, 100132365-100132921, 100133061-100133875, 100133989- |
| | | | | 100136823, 100136931-100137734, 100137872-100139043, |
| 13 | TSTD2 | NM_139246 | chr9 | 100365050-100367007, 100367148-100367763, 100367924-100368423, 100368544- |
| | | | | 100372626, 100372734-100373854, 100373982-100380037, 100380160-100387961, 100388280- |
| | | | | 100389678, 100389895-100395523, |
| 13 | NCBP1 | NM_002486 | chr9 | 100396198-100403074, 100403165-100403842, 100403945-100405484, 100405643- |
| | | | | 100407397, 100407507-100407891, 100408015-100409772, 100409844-100410288, 100410506- |
| | | | | 100412783, 100412883-100413590, 100413656-100416078, 100416191-100417160, 100417227- |
| | | | | 100417982, 100418047-100418291, 100418368-100420920, 100421026-100423226, 100423351- |
| | | | | 100424322, 100424427-100425234, 100425330-100426620, 100426726-100429043, 100429160- |
| | | | | 100431126, 100431257-100431873, 100431989-100433366, |
| 13 | XPA | NR_027302 | chr9 | 100437870-100444480, 100444713-100447203, 100447323-100449376, 100449544- |
| | | | | 100451814, 100451922-100455929, 100456042-100459401, |
| 13 | XPA | NM_000380 | chr9 | 100437870-100447203, 100447323-100449376, 100449544-100451814, 100451922- |
| | | | | 100455929, 100456042-100459401, |
| 13 | C9orf156 | NM_016481 | chr9 | 100667275-100672240, 100672899-100675681, 100675841-100678444, 100678621-100684698, |
| 14 | XPO1 | NM_003400 | chr2 | 61706102-61708318, 61708417-61709513, 61709675-61710090, 61710227-61711070, 61711241- |
| | | | | 61712901, 61713098-61715298, 61715407-61715721, 61715907-61717775, 61717912-61719168, 61719334- |
| | | | | 61719458, 61719617-61719700, 61719884-61720048, 61720189-61721027, 61721227-61722588, 61722749- |
| | | | | 61724012, 61724143-61725806, 61725928-61725998, 61726049-61726846, 61727030-61729129, 61729176- |
| | | | | 61729382, 61729446-61749744, 61749819-61753553, 61753657-61760905, 61761039-61764695, |
| 14 | RAB1A | NM_004161 | chr2 | 65315825-65316071, 65316205-65318115, 65318213-65325103, 65325201-65331866, 65331941-65357025, |
| 14 | RAB1A | NM_015543 | chr2 | 65315825-65325103, 65325201-65331866, 65331941-65357025, |
| 14 | MEIS1 | NM_002398 | chr2 | 66663001-66664867, 66665096-66666973, 66667117-66667692, 66667745-66668544, 66668597- |
| | | | | 66670032, 66670181-66691239, 66691353-66739279, 66739427-66775073, 66775152-66794583, 66794644- |
| | | | | 66795797, 66795889-66796180, 66796278-66798376, |
| 14 | PPP3R1 | NM_000945 | chr2 | 68408143-68413598, 68413785-68414385, 68414447-68415644, 68415823-68444222, 68444264-68479255, |
| 14 | GKN1 | NM_019617 | chr2 | 69201822-69204625, 69204681-69204767, 69204907-69206001, 69206114-69207043, 69207193-69207859, |
| 14 | GFPT1 | NM_002056 | chr2 | 69553366-69554044, 69554208-69555401, 69555571-69556427, 69556557-69556814, 69556931- |
| | | | | 69565028, 69565188-69565575, 69565698-69569282, 69569382-69573034, 69573132-69575301, 69575467- |
| | | | | 69577208, 69577316-69581619, 69581701-69583626, 69583690-69585459, 69585596-69586398, 69586459- |
| | | | | 69590675, 69590803-69597131, 69597241-69601136, 69601246-69614195, |
| 14 | C2orf78 | NM_001080474 | chr2 | 74011534-74040602, 74041354-74042196, |
| 15 | CIZ1 | NM_012127 | chr9 | 130928686-130929036, 130929160-130929372, 130929444-130931329, 130931481- |
| | | | | 130931683, 130931799-130932297, 130932387-130938628, 130938755-130939843, 130940043- |
| | | | | 130940646, 130940770-130940986, 130941695-130942692, 130942803-130942998, 130943094- |
| | | | | 130947824, 130948056-130950140, 130950214-130952606, 130952724-130952965, 130953142- |
| | | | | 130966463, |
| 15 | CIZ1 | NM_001131015 | chr9 | 130928686-130929036, 130929160-130929372, 130929444-130931329, 130931481- |
| | | | | 130931683, 130931799-130932297, 130932387-130938628, 130938755-130939843, 130940043- |
| | | | | 130940646, 130940770-130940986, 130941184-130941350, 130941695-130942692, 130942803- |
| | | | | 130942998, 130943094-130947824, 130948056-130950140, 130950214-130952606, 130952724- |
| | | | | 130952965, 130953142-130966463, |
| 15 | CIZ1 | NM_001131016 | chr9 | 130928686-130929036, 130929160-130929372, 130929444-130931329, 130931481- |
| | | | | 130931683, 130931799-130932297, 130932387-130938628, 130938755-130939843, 130940043- |
| | | | | 130940646, 130940770-130940986, 130941695-130942692, 130942803-130942998, 130943094- |
| | | | | 130947824, 130948056-130950140, 130950214-130952606, 130952724-130952965, 130953142- |
| | | | | 130953709, |
| 15 | CIZ1 | NM_001131018 | chr9 | 130928686-130929036, 130929160-130929372, 130929444-130931329, 130931481- |
| | | | | 130931683, 130931799-130932297, 130932387-130938628, 130938755-130939843, 130940043- |
| | | | | 130940646, 130940770-130940986, 130941184-130941350, 130941695-130942692, 130942803- |
| | | | | 130942998, 130943094-130947824, 130948056-130952606, 130952724-130952965, 130953142- |
| | | | | 130953709, |
| 15 | CIZ1 | NM_001131017 | chr9 | 130928686-130929036, 130929160-130929372, 130929444-130931329, 130931481- |
| | | | | 130931683, 130931799-130932297, 130932387-130938628, 130938755-130939843, 130940043- |
| | | | | 130940646, 130940770-130940986, 130941184-130941350, 130941695-130942692, 130942803- |
| | | | | 130942998, 130943079-130947824, 130948056-130950140, 130950214-130952606, 130952724- |
| | | | | 130952965, 130953142-130953709, |
| 15 | SPTAN1 | NM_003127 | chr9 | 131314976-131329015, 131329257-131331049, 131331177-131336952, 131337095- |
| | | | | 131337476, 131337625-131339100, 131339236-131339406, 131339553-131339629, 131339786- |
| | | | | 131340387, 131340525-131341914, 131342018-131343199, 131343339-131344059, 131344172- |
| | | | | 131344756, 131344836-131344971, 131345129-131345354, 131345561-131346065, 131346249- |
| | | | | 131346559, 131346805-131346998, 131347123-131348025, 131348245-131349883, 131349978- |
| | | | | 131351086, 131351223-131353754, 131353905-131355260, 131355322-131356452, 131356653- |
| | | | | 131360677, 131360784-131361240, 131361266-131362357, 131362395-131365820, 131365870- |
| | | | | 131366603, 131366697-131367311, 131367458-131367573, 131367757-131369881, 131369983- |
| | | | | 131370129, 131370329-131370407, 131370556-131371151, 131371257-131371399, 131371564- |
| | | | | 131373991, 131374125-131374386, 131374526-131375658, 131375765-131377909, 131378120- |
| | | | | 131379917, 131380040-131380264, 131380388-131381148, 131381283-131383435, 131383536- |
| | | | | 131386605, 131386767-131387380, 131387459-131388046, 131388258-131388668, 131388967- |
| | | | | 131389663, 131389778-131390202, 131390222-131392598, 131392655-131394404, 131394603- |
| | | | | 131394708, 131394764-131394849, 131394998-131395085, 131395235-131395486, |
| 15 | SPTAN1 | NM_001130438 | chr9 | 131314976-131329015, 131329257-131331049, 131331177-131336952, 131337095- |
| | | | | 131337476, 131337625-131339100, 131339236-131339406, 131339553-131339629, 131339786- |
| | | | | 131340387, 131340525-131341914, 131342018-131343199, 131343339-131344059, 131344172- |
| | | | | 131344756, 131344836-131344971, 131345129-131345354, 131345561-131346065, 131346249- |
| | | | | 131346559, 131346805-131346998, 131347123-131348025, 131348245-131349883, 131349978- |
| | | | | 131351086, 131351223-131353754, 131353905-131355260, 131355322-131356452, 131356653- |
| | | | | 131360677, 131360784-131361240, 131361266-131362357, 131362395-131365820, 131365870- |
| | | | | 131366603, 131366697-131367311, 131367458-131367573, 131367757-131369881, 131369983- |
| | | | | 131370129, 131370329-131370407, 131370556-131371151, 131371257-131371399, 131371564- |
| | | | | 131371928, 131371945-131373991, 131374125-131374386, 131374526-131375658, 131375765- |
| | | | | 131377909, 131378120-131379917, 131380040-131380264, 131380388-131381148, 131381283- |
| | | | | 131383435, 131383536-131386605, 131386767-131387380, 131387459-131388046, 131388258- |
| | | | | 131388668, 131388967-131389663, 131389778-131390202, 131390222-131392598, 131392655- |
| | | | | 131394404, 131394603-131394708, 131394764-131394849, 131394998-131395085, 131395235- |
| | | | | 131395486, |
| 15 | SPTAN1 | NM_001195532 | chr9 | 131314976-131329015, 131329257-131331049, 131331177-131336952, 131337095- |
| | | | | 131337476, 131337625-131339100, 131339236-131339406, 131339553-131339629, 131339786- |
| | | | | 131340387, 131340525-131341914, 131342018-131343199, 131343339-131344059, 131344172- |
| | | | | 131344756, 131344836-131344971, 131345129-131345354, 131345561-131346065, 131346249- |
| | | | | 131346559, 131346805-131346998, 131347123-131348025, 131348245-131349883, 131349978- |
| | | | | 131351086, 131351223-131353754, 131353905-131356452, 131356653-131360677, 131360784- |
| | | | | 131361240, 131361266-131362357, 131362395-131365820, 131365870-131366603, 131366697- |
| | | | | 131367311, 131367458-131367573, 131367757-131369881, 131369983-131370129, 131370329- |
| | | | | 131370407, 131370556-131371151, 131371257-131371399, 131371564-131373991, 131374125- |
| | | | | 131374386, 131374526-131375658, 131375765-131377909, 131378120-131379917, 131380040- |
| | | | | 131380264, 131380388-131381148, 131381283-131383435, 131383536-131386605, 131386767- |
| | | | | 131387380, 131387459-131388046, 131388258-131388668, 131388967-131389663, 131389778- |
| | | | | 131390202, 131390222-131392598, 131392655-131394404, 131394603-131394708, 131394764- |
| | | | | 131394849, 131394998-131395085, 131395235-131395486, |
| 15 | SET | NM_003011 | chr9 | 131451935-131453447, 131453507-131454135, 131454280-131454939, 131455045- |
| | | | | 131455145, 131455261-131455915, 131456088-131456173, 131456322-131456918, |
| 15 | SET | NM_001122821 | chr9 | 131446287-131453447, 131453507-131454135, 131454280-131454939, 131455045- |
| | | | | 131455145, 131455261-131455915, 131456088-131456173, 131456322-131456918, |
| 15 | ENDOG | NM_004435 | chr9 | 131581465-131583016, 131583128-131584605, |
| 15 | LRRC8A | NM_001127245 | chr9 | 131644530-131669434, 131671601-131678373, |
| 15 | LRRC8A | NM_019594 | chr9 | 131644530-131648292, 131648401-131669434, 131671601-131678373, |
| 15 | LRRC8A | NM_001127244 | chr9 | 131645189-131648292, 131648401-131669434, 131671601-131678373, |
| 15 | PHYHD1 | NM_001100877 | chr9 | 131684636-131689315, 131689476-131696059, 131696137-131696288, 131696338- |
| | | | | 131698725, 131698783-131700034, 131700058-131702646, 131702777-131702876, 131702995- |
| | | | | 131703722, 131703851-131703945, |
| 15 | PHYHD1 | NM_001100876 | chr9 | 131683948-131684207, 131684327-131684560, 131684636-131689315, 131689476- |
| | | | | 131696059, 131696137-131696288, 131696338-131698725, 131698783-131698860, 131698925- |
| | | | | 131700034, 131700058-131702646, 131702777-131702876, 131702995-131703722, 131703851- |
| | | | | 131703945, |
| 15 | PHYHD1 | NM_174933 | chr9 | 131683948-131684207, 131684327-131684560, 131684636-131689315, 131689476- |
| | | | | 131696059, 131696137-131696288, 131696338-131698725, 131698783-131698860, 131698925- |
| | | | | 131702646, 131702777-131702876, 131702995-131703722, 131703851-131703945, |
| 15 | NUP188 | NM_015354 | chr9 | 131710031-131711466, 131711523-131715050, 131715126-131718627, 131718714- |
| | | | | 131719229, 131719312-131720287, 131720334-131721079, 131721174-131721292, 131721414- |
| | | | | 131730783, 131730997-131731677, 131731794-131733035, 131733238-131735437, 131735529- |
| | | | | 131741539, 131741607-131742883, 131743005-131743541, 131743670-131744826, 131744981- |
| | | | | 131745179, 131745308-131745570, 131745660-131745751, 131745830-131747177, 131747294- |
| | | | | 131748835, 131748958-131749086, 131749155-131749866, 131749997-131750324, 131750466- |
| | | | | 131752397, 131752506-131755474, 131755683-131755802, 131755974-131756650, 131756722- |
| | | | | 131757133, 131757203-131757596, 131757794-131760428, 131760508-131760815, 131760903- |
| | | | | 131761449, 131761597-131761901, 131762085-131763806, 131764009-131764161, 131764256- |
| | | | | 131765094, 131765255-131765594, 131765734-131767424, 131767501-131767580, 131767810- |
| | | | | 131767922, 131768056-131768327, 131768403-131768516, 131768648-131768779, |
| 15 | SH3GLB2 | NM_020145 | chr9 | 131771071-131771383, 131771626-131772048, 131772151-131772397, 131772489- |
| | | | | 131772947, 131772973-131774513, 131774578-131776688, 131776783-131777048, 131777184- |
| | | | | 131783368, 131783499-131784559, 131784703-131790369, |
| 15 | TOR1A | NM_000113 | chr9 | 132576502-132580797, 132580927-132581022, 132581200-132584858, 132585126-132586185, |
| 15 | USP20 | NM_001110303 | chr9 | 132597779-132612090, 132612204-132614817, 132614916-132618584, 132618640- |
| | | | | 132620327, 132620392-132620744, 132620878-132623214, 132623313-132623820, 132623892- |
| | | | | 132625463, 132625579-132627580, 132627661-132630282, 132630729-132631139, 132631282- |
| | | | | 132631587, 132631705-132631949, 132632124-132632730, 132632827-132635759, 132635795- |
| | | | | 132635976, 132636024-132636853, 132637036-132637106, 132637235-132637587, 132637759- |
| | | | | 132637837, 132637921-132638407, 132638518-132640615, 132640720-132641850, 132641924- |
| | | | | 132642390, 132642569-132642744, |
| 15 | USP20 | NM_006676 | chr9 | 132597779-132612090, 132612204-132614817, 132614916-132618584, 132618640- |
| | | | | 132620327, 132620392-132620744, 132620878-132623214, 132623313-132623820, 132623892- |
| | | | | 132625463, 132625579-132627580, 132627661-132630282, 132630729-132631139, 132631282- |
| | | | | 132631587, 132631705-132631949, 132632124-132632730, 132632827-132635759, 132635795- |
| | | | | 132635976, 132636024-132636853, 132637036-132637106, 132637235-132637587, 132637759- |
| | | | | 132637837, 132637921-132638407, 132638518-132640615, 132640720-132641850, 132641924- |
| | | | | 132642390, |
| 15 | USP20 | NM_001008563 | chr9 | 132597877-132612090, 132612204-132614817, 132614916-132618584, 132618640- |
| | | | | 132620327, 132620392-132620744, 132620878-132623214, 132623313-132623820, 132623892- |
| | | | | 132625463, 132625579-132627580, 132627661-132630282, 132630729-132631139, 132631282- |
| | | | | 132631587, 132631705-132631949, 132632124-132632730, 132632827-132635759, 132635795- |
| | | | | 132635976, 132636024-132636853, 132637036-132637106, 132637235-132637587, 132637759- |
| | | | | 132637837, 132637921-132638407, 132638518-132640615, 132640720-132641850, 132641924- |
| | | | | 132642390, 132642569-132642744, |
| 15 | GPR107 | NM_020960 | chr9 | 132816353-132837913, 132838029-132838689, 132838742-132839576, 132839658- |
| | | | | 132841907, 132842049-132845842, 132845882-132848697, 132848756-132853153, 132853263- |
| | | | | 132854525, 132854661-132861734, 132861812-132862906, 132862982-132863383, 132863503- |
| | | | | 132866697, 132866830-132869773, 132869819-132887192, 132887244-132889760, 132889846- |
| | | | | 132890917, 132891041-132897302, |
| 15 | GPR107 | NM_001136557 | chr9 | 132816353-132837913, 132838029-132838689, 132838742-132839576, 132839658- |
| | | | | 132841907, 132842049-132845842, 132845882-132848697, 132848756-132853153, 132853263- |
| | | | | 132854525, 132854661-132861734, 132861812-132862906, 132862982-132863383, 132863503- |
| | | | | 132866697, 132866830-132869773, 132869819-132872647, 132872706-132876854, 132876943- |
| | | | | 132887192, 132887244-132889760, 132889846-132890917, 132891041-132897302, |
| 15 | GPR107 | NM_001136558 | chr9 | 132816353-132837913, 132838029-132838689, 132838742-132839576, 132839658- |
| | | | | 132841907, 132842049-132845842, 132845882-132848697, 132848756-132853153, 132853263- |
| | | | | 132854525, 132854661-132861734, 132861812-132862906, 132862982-132863383, 132863503- |
| | | | | 132866697, 132866830-132869773, 132869819-132872647, 132872706-132887192, 132887244- |
| | | | | 132889760, 132889846-132890917, 132891041-132897302, |
| 15 | ABL1 | NM_007313 | chr9 | 133589843-133729449, 133729625-133730186, 133730484-133738148, 133738423- |
| | | | | 133747514, 133747601-133748245, 133748425-133750253, 133750440-133753800, 133753955- |
| | | | | 133755453, 133755545-133755885, 133756052-133759354, |
| 15 | ABL1 | NM_005157 | chr9 | 133710913-133729449, 133729625-133730186, 133730484-133738148, 133738423- |
| | | | | 133747514, 133747601-133748245, 133748425-133750253, 133750440-133753800, 133753955- |
| | | | | 133755453, 133755545-133755885, 133756052-133759354, |
| 15 | TTF1 | NM_007344 | chr9 | 135251556-135254444, 135254532-135261941, 135262009-135263524, 135263616- |
| | | | | 135265982, 135266219-135267461, 135267594-135271818, 135271899-135273526, 135273714- |
| | | | | 135275420, 135275646-135276840, 135278216-135282175, |
| 15 | AK8 | NM_152572 | chr9 | 135601313-135602839, 135602922-135668019, 135668163-135690023, 135690115- |
| | | | | 135698590, 135698724-135702239, 135702442-135703398, 135703472-135704030, 135704114- |
| | | | | 135730242, 135730313-135739050, 135739166-135742289, 135742341-135750500, 135750587- |
| | | | | 135753557, |
| 15 | C9orf9 | NM_018956 | chr9 | 135754402-135759296, 135759479-135762754, 135762959-135763675, 135763825-135765342, |
| 15 | TSC1 | NM_001162427 | chr9 | 135772142-135772569, 135772733-135772808, 135772998-135776100, 135776225- |
| | | | | 135776974, 135777087-135777990, 135778175-135779036, 135779205-135779796, 135779842- |
| | | | | 135780966, 135781527-135782116, 135782223-135782686, 135782758-135785956, 135786080- |
| | | | | 135786387, 135786501-135786838, 135786956-135787667, 135787845-135796748, 135796824- |
| | | | | 135797204, 135797361-135798733, 135798880-135802586, 135802692-135804152, 135804340- |
| | | | | 135810418, 135810483-135819928, |
| 15 | TSC1 | NM_000368 | chr9 | 135772142-135772569, 135772733-135772808, 135772998-135776100, 135776225- |
| | | | | 135776974, 135777087-135777990, 135778175-135779036, 135779205-135779796, 135779842- |
| | | | | 135780966, 135781527-135782116, 135782223-135782686, 135782758-135785956, 135786080- |
| | | | | 135786387, 135786501-135786838, 135786956-135787667, 135787845-135796748, 135796824- |
| | | | | 135797204, 135797361-135798733, 135798880-135800972, 135801127-135802586, 135802692- |
| | | | | 135804152, 135804340-135810418, 135810483-135819928, |
| 15 | TSC1 | NM_001162426 | chr9 | 135772142-135772569, 135772733-135772808, 135772998-135776100, 135776225- |
| | | | | 135776974, 135777087-135777990, 135778175-135779036, 135779205-135779796, 135779842- |
| | | | | 135780966, 135781527-135782116, 135782223-135782686, 135782758-135785956, 135786077- |
| | | | | 135786387, 135786501-135786838, 135786956-135787667, 135787845-135796748, 135796824- |
| | | | | 135797204, 135797361-135798733, 135798880-135800972, 135801127-135802586, 135802692- |
| | | | | 135804152, 135804340-135810418, 135810483-135819928, |
| 15 | SURF1 | NM_003172 | chr9 | 136218838-136218914, 136218998-136219299, 136219464-136219547, 136219622- |
| | | | | 136220602, 136220796-136221512, 136221597-136221677, 136221813-136223122, 136223176- |
| | | | | 136223274, |
| 15 | SURF2 | NM_017503 | chr9 | 136223547-136223788, 136223945-136224585, 136224691-136226824, 136227006- |
| | | | | 136227139, 136227311-136227930, |
| 15 | ADAMTSL2 | NM_001145320 | chr9 | 136397693-136401683, 136401925-136402525, 136402670-136403469, 136403547- |
| | | | | 136404891, 136404996-136405718, 136405866-136405998, 136406124-136409590, 136409673- |
| | | | | 136412158, 136412336-136419477, 136419816-136420678, 136421053-136426318, 136426418- |
| | | | | 136432056, 136432185-136433393, 136433609-136433723, 136433881-136434528, 136434701- |
| | | | | 136435451, 136435630-136438963, 136439110-136439866, |
| 15 | ADAMTSL2 | NM_014694 | chr9 | 136400040-136401683, 136401925-136402525, 136402670-136403469, 136403547- |
| | | | | 136404891, 136404996-136405718, 136405866-136405998, 136406124-136409590, 136409673- |
| | | | | 136412158, 136412336-136419477, 136419816-136420678, 136421053-136426318, 136426418- |
| | | | | 136432056, 136432185-136433393, 136433609-136433723, 136433881-136434528, 136434701- |
| | | | | 136435451, 136435630-136438963, 136439110-136439866, |
| 16 | ARPP21 | NM_001025069 | chr3 | 35721194-35721277, 35721453-35723204, 35723373-35724338, 35724382-35725216, 35725308-35725511, |
| 16 | ARPP21 | NM_016300 | chr3 | 35683901-35721277, 35721453-35723204, 35723373-35724338, 35724382-35725216, 35725308- |
| | | | | 35729229, 35729376-35730797, 35730878-35731571, 35731633-35732355, 35732498-35748464, 35748575- |
| | | | | 35750459, 35750563-35756929, 35756969-35758788, 35758850-35763095, 35763327-35770793, 35771029- |
| | | | | 35778668, 35778855-35780807, 35781070-35785329, 35785458-35833872, 35834023-35835191, |
| 16 | ARPP21 | NM_198399 | chr3 | 35722663-35723204, 35723373-35724338, 35724382-35725216, 35725308-35725511, |
| 16 | ARPP21 | NM_001025068 | chr3 | 35721185-35721277, 35721453-35723204, 35723373-35724338, 35724382-35725216, 35725308-35725511, |
| 16 | VILL | NM_015873 | chr3 | 38035224-38035387, 38035464-38035750, 38035958-38037228, 38037339-38038566, 38038679- |
| | | | | 38038972, 38039177-38039579, 38039660-38039755, 38039856-38040401, 38040542-38040828, 38040931- |
| | | | | 38042945, 38043085-38043191, 38043352-38043885, 38044067-38044657, 38044805-38045744, 38045891- |
| | | | | 38047281, 38047466-38047670, 38047744-38047938, 38048192-38048431, |
| 16 | DLEC1 | NM_007335 | chr3 | 38081128-38087032, 38087185-38101231, 38101344-38103658, 38103860-38104070, 38104293- |
| | | | | 38105330, 38105411-38125647, 38125737-38126763, 38126939-38127730, 38127869-38129785, 38129880- |
| | | | | 38134279, 38134372-38135094, 38135259-38136368, 38136563-38137377, 38137438-38138058, 38138229- |
| | | | | 38138671, 38138767-38138996, 38139129-38139233, 38139394-38141775, 38141917-38149073, 38149229- |
| | | | | 38149894, 38150007-38150921, 38151054-38151588, 38151773-38153166, 38153239-38153699, 38153853- |
| | | | | 38155831, 38155952-38156472, 38156545-38157942, 38158150-38158262, 38158381-38158464, 38158592- |
| | | | | 38158697, 38158794-38159016, 38159103-38159295, 38159516-38161937, 38162101-38163117, 38163273- |
| | | | | 38163560, 38163686-38163778, |
| 16 | DLEC1 | NM_007337 | chr3 | 38081128-38087032, 38087185-38101231, 38101344-38103658, 38103860-38104070, 38104293- |
| | | | | 38105330, 38105411-38125647, 38125737-38126763, 38126939-38127730, 38127869-38129785, 38129880- |
| | | | | 38134279, 38134372-38135094, 38135259-38136368, 38136563-38137377, 38137438-38138058, 38138229- |
| | | | | 38138671, 38138767-38138996, 38139129-38139233, 38139394-38141775, 38141917-38149073, 38149229- |
| | | | | 38149894, 38150007-38150921, 38151054-38151588, 38151773-38153166, 38153239-38153699, 38153853- |
| | | | | 38155831, 38155952-38156472, 38156545-38157942, 38158150-38158262, 38158381-38158464, 38158592- |
| | | | | 38158697, 38158794-38159016, 38159103-38159295, 38159516-38161937, 38162101-38163117, 38163273- |
| | | | | 38163778, |
| 16 | CX3CR1 | NM_001171171 | chr3 | 39308010-39322532, |
| 16 | CX3CR1 | NM_001171172 | chr3 | 39308010-39321738, |
| 16 | CX3CR1 | NM_001171174 | chr3 | 39308010-39323098, |
| 16 | CX3CR1 | NM_001337 | chr3 | 39308010-39321443, |
| 16 | ZNF619 | NM_001145093 | chr3 | 40518849-40519407, 40519495-40523352, 40523508-40524077, 40524196-40528295, |
| 16 | ZNF619 | NR_026746 | chr3 | 40518849-40519407, 40519495-40523352, 40523508-40523689, 40523841-40524077, 40524196-40528295, |
| 16 | ZNF619 | NM_001145082 | chr3 | 40518849-40519407, 40519495-40523352, 40523508-40523719, 40523841-40524077, 40524196-40528295, |
| 16 | ZNF619 | NM_001145083 | chr3 | 40518849-40519407, 40519495-40524077, 40524196-40528295, |
| 16 | TRAK1 | NM_001042646 | chr3 | 42133053-42166910, 42167107-42218304, 42218383-42226175, 42226294-42229535, 42229638- |
| | | | | 42230506, 42230617-42233048, 42233129-42234565, 42234698-42235314, 42235391-42236294, 42236434- |
| | | | | 42240667, 42240746-42242308, 42242547-42243926, 42244245-42251257, 42251478-42260984, 42261089- |
| | | | | 42264432, |
| 16 | TRAK1 | NM_014965 | chr3 | 42201966-42218304, 42218383-42226175, 42226294-42229535, 42229638-42230506, 42230617- |
| | | | | 42233048, 42233129-42234565, 42234698-42235314, 42235391-42236294, 42236434-42240667, 42240746- |
| | | | | 42242308, 42242547-42243926, 42244245-42251257, |
| 17 | LPPR2 | NM_022737 | chr19 | 11466249-11467476, 11467653-11468334, 11468416-11470206, 11470397-11470495, 11470633- |
| | | | | 11471891, 11472165-11473187, 11473366-11474387, 11474496-11474835, 11474889-11475021, |
| 17 | LPPR2 | NM_001170635 | chr19 | 11466249-11467476, 11467653-11468334, 11468416-11470281, 11470397-11470495, 11470633- |
| | | | | 11471891, 11472165-11473187, 11473366-11474387, 11474512-11474835, 11474889-11475021, |
| 17 | EPOR | NM_000121 | chr19 | 11489272-11489365, 11489455-11491558, 11491648-11491730, 11491886-11492366, 11492526- |
| | | | | 11492604, 11492782-11493771, 11493909-11494767, |
| 17 | EPOR | NR_033663 | chr19 | 11489272-11489365, 11489455-11491558, 11491648-11491730, 11491886-11492366, 11492526- |
| | | | | 11492604, 11492782-11493521, 11493909-11494767, |
| 17 | ZNF627 | NM_145295 | chr19 | 11708446-11725340, 11725469-11725640, 11725703-11727508, |
| 17 | ZNF491 | NM_152356 | chr19 | 11909543-11915355, 11915483-11916760, |
| 17 | ZNF763 | NM_001012753 | chr19 | 12076027-12087842, 12087980-12088184, 12088247-12088929, |
| 17 | ZNF433 | NM_001080411 | chr19 | 12127482-12128689, 12128752-12128979, 12129117-12146350, |
| 17 | ZNF788 | NR_027049 | chr19 | 12203286-12221118, 12221247-12222059, |
| 17 | ZNF625 | NM_145233 | chr19 | 12257040-12258208, 12258271-12258493, 12258618-12267369, |
| 17 | ZNF136 | NM_003437 | chr19 | 12274022-12296598, 12296727-12296927, 12296990-12297383, |
| 17 | ZNF44 | NM_016264 | chr19 | 12384879-12385786, 12385849-12386769, 12386898-12405505, |
| 17 | ZNF44 | NM_001164276 | chr19 | 12384879-12385786, 12385849-12386769, 12386898-12404045, 12404191-12405505, |
| 17 | ZNF563 | NM_145276 | chr19 | 12430648-12432084, 12432147-12433397, 12433526-12444292, |
| 17 | ZNF799 | NM_001080821 | chr19 | 12503021-12503415, 12503478-12504109, 12504238-12511880, |
| 17 | ZNF709 | NM_152601 | chr19 | 12576548-12577274, 12577334-12577536, 12577665-12595467, |
| 17 | ZNF564 | NM_144976 | chr19 | 12638731-12639116, 12639179-12639382, 12639511-12662142, |
| 17 | ZNF490 | NM_020714 | chr19 | 12692539-12693662, 12693725-12694242, 12694371-12719970, 12720017-12721376, |
| 17 | ZNF791 | NM_153358 | chr19 | 12721897-12734512, 12734641-12735462, 12735525-12738533, |
| 17 | HOOK2 | NM_013312 | chr19 | 12874246-12874340, 12874414-12874480, 12874593-12875626, 12875732-12875811, 12875896- |
| | | | | 12876502, 12876539-12876641, 12876649-12876739, 12876829-12876915, 12877055-12878207, 12878279- |
| | | | | 12878628, 12878718-12878825, 12878938-12880693, 12880897-12881744, 12881887-12881971, 12882134- |
| | | | | 12882209, 12882292-12883002, 12883067-12883425, 12883495-12883592, 12883727-12883806, 12883859- |
| | | | | 12885481, 12885556-12885624, 12885712-12886217, |
| 17 | HOOK2 | NM_001100176 | chr19 | 12874246-12874340, 12874414-12874480, 12874593-12875626, 12875732-12875811, 12875896- |
| | | | | 12876502, 12876539-12876739, 12876829-12876915, 12877055-12878207, 12878279-12878628, 12878718- |
| | | | | 12878825, 12878938-12880693, 12880897-12881744, 12881887-12881971, 12882134-12882209, 12882292- |
| | | | | 12883002, 12883067-12883425, 12883495-12883592, 12883727-12883806, 12883859-12885481, 12885556- |
| | | | | 12885624, 12885712-12886217, |
| 17 | LYL1 | NM_005583 | chr19 | 13210549-13211469, 13211563-13211649, 13212010-13213343, |
| 18 | ERRFI1 | NM_018948 | chr1 | 8074457-8075366, 8075445-8075553, 8075753-8086216, |
| 18 | ENO1 | NM_001428 | chr1 | 8921489-8922944, 8923005-8923292, 8923403-8923948, 8924152-8925342, 8925542-8926336, 8926561- |
| | | | | 8927174, 8927310-8928045, 8928117-8930509, 8930570-8931948, 8932046-8934881, 8934977-8938637, |
| 18 | KIF1B | NM_183416 | chr1 | 10270937-10292306, 10292493-10316303, 10316382-10318549, 10318731-10321961, 10322029- |
| | | | | 10327436, 10327617-10328208, 10328322-10331558, 10331638-10332297, 10332365-10335484, 10335562- |
| | | | | 10336377, 10336458-10338042, 10338187-10342456, 10342592-10351138, 10351220-10352103, 10352181- |
| | | | | 10355142, 10355224-10355716, 10355825-10356638, 10356724-10356953, 10357136-10357230, 10357305- |
| | | | | 10363219, |
| 18 | KIF1B | NM_015074 | chr1 | 10270937-10292306, 10292493-10316303, 10316382-10318549, 10318731-10321961, 10322029- |
| | | | | 10327436, 10327617-10328208, 10328322-10331558, 10331638-10332297, 10332365-10335484, 10335562- |
| | | | | 10336377, 10336458-10338042, 10338187-10342456, 10342592-10351138, 10351220-10352103, 10352181- |
| | | | | 10355142, 10355224-10355716, 10355825-10356638, 10356724-10356953, 10357136-10357230, 10357305- |
| | | | | 10380099, 10380195-10381765, 10381916-10383940, 10384121-10384814, 10384954-10386167, 10386418- |
| | | | | 10394576, 10394697-10396713, 10396801-10397130, 10397262-10397427, 10397592-10399825, 10399918- |
| | | | | 10402106, 10402227-10403288, 10403346-10405901, 10406012-10407817, 10407886-10408705, 10408792- |
| | | | | 10412687, 10412795-10420985, 10421102-10421748, 10421884-10423339, 10423403-10425156, 10425304- |
| | | | | 10425465, 10425707-10428523, 10428597-10431197, 10431321-10434372, 10434524-10434910, 10435105- |
| | | | | 10435311, 10435432-10436601, |
| 18 | VPS13D | NM_018156 | chr1 | 12290178-12294246, 12294421-12302568, 12302648-12304301, 12304494-12304592, 12304675- |
| | | | | 12309278, 12309397-12313777, 12313884-12316388, 12316561-12317042, 12317145-12317990, 12318161- |
| | | | | 12320748, 12320852-12321003, 12321207-12321956, 12322138-12326936, 12327069-12327900, 12327978- |
| | | | | 12328761, 12328934-12331049, 12331182-12333058, 12333193-12335880, 12338096-12339554, 12339708- |
| | | | | 12342760, 12343794-12348278, 12348371-12351053, 12351182-12353579, 12353762-12359257, 12359442- |
| | | | | 12364561, 12364786-12368486, 12368699-12371509, 12371683-12371868, 12371983-12374170, 12374385- |
| | | | | 12378127, 12378395-12379552, 12379688-12381864, 12382022-12382591, 12382804-12383761, 12383838- |
| | | | | 12387703, 12387912-12389884, 12389976-12393281, 12393424-12395760, 12395885-12401835, 12401943- |
| | | | | 12402954, 12403109-12405429, 12405567-12406660, 12406710-12408878, 12409031-12409219, 12409432- |
| | | | | 12414029, 12414279-12415954, 12416148-12416453, 12416582-12418513, 12418658-12422774, 12422907- |
| | | | | 12423126, 12423304-12428521, 12428647-12429520, 12429758-12433803, 12433914-12438480, 12438646- |
| | | | | 12439540, 12439651-12443033, 12443215-12445317, 12445433-12446242, 12446394-12460236, 12460388- |
| | | | | 12461659, 12461758-12463876, 12464027-12475138, 12475275-12476711, 12476881-12516052, 12516187- |
| | | | | 12520254, 12520452-12557552, 12557686-12566905, 12567148-12568945, |
| 18 | VPS13D | NM_015378 | chr1 | 12290178-12294246, 12294421-12302568, 12302648-12304301, 12304494-12304592, 12304675- |
| | | | | 12309278, 12309397-12313777, 12313884-12316388, 12316561-12317042, 12317145-12317990, 12318161- |
| | | | | 12320748, 12320852-12321003, 12321207-12321956, 12322138-12326936, 12327069-12327900, 12327978- |
| | | | | 12328761, 12328934-12331049, 12331182-12333058, 12333193-12335880, 12338096-12339554, 12339708- |
| | | | | 12342760, 12343794-12348278, 12348371-12351053, 12351182-12353579, 12353762-12359257, 12359442- |
| | | | | 12364561, 12364786-12368486, 12368699-12371509, 12371683-12371868, 12371983-12374170, 12374385- |
| | | | | 12378127, 12378395-12379552, 12379688-12381864, 12382022-12382591, 12382804-12383761, 12383838- |
| | | | | 12387703, 12387912-12389884, 12389976-12393281, 12393424-12395760, 12395885-12398286, 12398363- |
| | | | | 12401835, 12401943-12402954, 12403109-12405429, 12405567-12406660, 12406710-12408878, 12409031- |
| | | | | 12409219, 12409432-12414029, 12414279-12415954, 12416148-12416453, 12416582-12418513, 12418658- |
| | | | | 12422774, 12422907-12423126, 12423304-12428521, 12428647-12429520, 12429758-12433803, 12433914- |
| | | | | 12438480, 12438646-12439540, 12439651-12443033, 12443215-12445317, 12445433-12446242, 12446394- |
| | | | | 12460236, 12460388-12461659, 12461758-12463876, 12464027-12475138, 12475275-12476711, 12476881- |
| | | | | 12516052, 12516187-12520254, 12520452-12557552, 12557686-12566905, 12567148-12568945, |
| 18 | DHRS3 | NM_004753 | chr1 | 12628454-12632754, 12632882-12638744, 12638985-12639319, 12639441-12640549, 12640695-12677157, |
| 18 | PRAMEF11 | NM_001146344 | chr1 | 12885362-12887106, 12887690-12888355, 12888665-12891208, |
| 19 | PYY | NM_004160 | chr17 | 42030349-42030475, 42030558-42030662, 42030852-42031216, 42031344-42035304, 42035397- |
| | | | | 42043654, 42043901-42081757, |
| 19 | NAGS | NM_153006 | chr17 | 42082458-42083003, 42083280-42083390, 42083606-42083895, 42084078-42084689, 42084863- |
| | | | | 42084957, 42085142-42085814, |
| 19 | TMEM101 | NM_032376 | chr17 | 42089605-42090375, 42090524-42091723, 42091906-42092182, |
| 19 | TMUB2 | NM_001076674 | chr17 | 42264478-42265042, 42265112-42266388, 42266957-42267867, |
| 19 | TMUB2 | NM_024107 | chr17 | 42264478-42266388, 42266957-42267867, |
| 19 | TMUB2 | NM_177441 | chr17 | 42265112-42266301, 42266957-42267867, |
| 19 | UBTF | NM_014233 | chr17 | 42284736-42284820, 42284966-42285065, 42285139-42285230, 42285280-42286718, 42286910- |
| | | | | 42287011, 42287102-42287490, 42287603-42287684, 42287842-42288158, 42288316-42288405, 42288521- |
| | | | | 42288656, 42288700-42288972, 42289116-42289239, 42289375-42289710, 42289823-42290185, 42290308- |
| | | | | 42290584, 42290651-42293020, 42293178-42293272, 42293358-42293895, 42294073-42295538, |
| 19 | UBTF | NM_001076684 | chr17 | 42284736-42284820, 42284966-42285065, 42285139-42285230, 42285280-42286718, 42286910- |
| | | | | 42287011, 42287102-42287490, 42287603-42287684, 42287842-42288158, 42288316-42288405, 42288521- |
| | | | | 42288656, 42288700-42288972, 42289116-42289239, 42289375-42290185, 42290308-42290584, 42290651- |
| | | | | 42293020, 42293178-42293272, 42293358-42293895, 42294073-42295538, 42295665-42296811, |
| 19 | UBTF | NM_001076683 | chr17 | 42284736-42284820, 42284966-42285065, 42285139-42285230, 42285280-42286718, 42286910- |
| | | | | 42287011, 42287102-42287490, 42287603-42287684, 42287842-42288158, 42288316-42288405, 42288521- |
| | | | | 42288656, 42288700-42288972, 42289116-42289239, 42289375-42290185, 42290308-42290584, 42290651- |
| | | | | 42293020, 42293178-42293272, 42293358-42293895, 42294073-42295538, 42295665-42298106, |
| 19 | PLCD3 | NM_133373 | chr17 | 43190073-43190229, 43190381-43190488, 43190626-43190805, 43190974-43191616, 43191735- |
| | | | | 43192460, 43192612-43192709, 43192858-43193997, 43194152-43195359, 43195506-43195656, 43195860- |
| | | | | 43196180, 43196411-43197690, 43197822-43198267, 43198498-43198576, 43198740-43209613, |
| 19 | ACBD4 | NM_001135704 | chr17 | 43210031-43212706, 43212964-43213207, 43213321-43213473, 43213600-43213865, 43213988- |
| | | | | 43214057, 43214144-43214384, 43214507-43214733, 43214822-43215087, 43215198-43215278, 43215356- |
| | | | | 43216386, 43216528-43220808, |
| 19 | ACBD4 | NM_001135707 | chr17 | 43213321-43213473, 43213600-43213865, 43213988-43214057, 43214144-43214384, 43214507- |
| | | | | 43214733, 43214822-43215125, 43215198-43215288, 43215356-43216386, 43216528-43219948, |
| 19 | ACBD4 | NM_024722 | chr17 | 43213600-43213865, 43213988-43214057, 43214144-43214384, 43214507-43214733, 43214822- |
| | | | | 43215125, 43215198-43215278, 43215356-43216386, 43216528-43220808, |
| 19 | ACBD4 | NM_001135706 | chr17 | 43213321-43213473, 43213600-43213865, 43213988-43214057, 43214144-43214384, 43214507- |
| | | | | 43214733, 43214822-43215087, 43215198-43215278, 43215356-43216386, 43216528-43220808, |
| 19 | ACBD4 | NM_001135705 | chr17 | 43213321-43213473, 43213600-43213865, 43213988-43214057, 43214144-43214384, 43214507- |
| | | | | 43214733, 43214822-43215125, 43215198-43215278, 43215356-43216386, 43216528-43220808, |
| 19 | MAP3K14 | NM_003954 | chr17 | 43342168-43342528, 43342631-43343902, 43344049-43344457, 43344566-43344771, 43345127- |
| | | | | 43347778, 43347931-43348423, 43348589-43350868, 43350975-43351488, 43351622-43351829, 43351961- |
| | | | | 43362177, 43362317-43363796, 43364412-43364518, 43364731-43366600, 43366672-43367854, 43368132- |
| | | | | 43394324, |
| 19 | LOC100128977 | NR_024559 | chr17 | 43921528-43972844, |
| 19 | MAPT | NM_001123066 | chr17 | 43972053-44039685, 44039837-44049223, 44049312-44051749, 44051838-44055739, 44055807- |
| | | | | 44060542, 44061297-44064404, 44064462-44067242, 44067442-44068824, 44068953-44071288, 44071344- |
| | | | | 44073763, 44074031-44087674, 44087769-44091607, 44091691-44095982, 44096097-44101320, |
| 19 | MAPT | NM_001123067 | chr17 | 43972053-44039685, 44039837-44049223, 44049312-44055739, 44055807-44064404, 44064462- |
| | | | | 44068824, 44068953-44073763, 44074031-44087674, 44087769-44091607, 44091691-44095982, 44096097- |
| | | | | 44101320, |
| 19 | MAPT | NM_016835 | chr17 | 43972053-44039685, 44039837-44049223, 44049312-44051749, 44051838-44055739, 44055807- |
| | | | | 44060542, 44061297-44064404, 44064462-44067242, 44067442-44068824, 44068953-44073763, 44074031- |
| | | | | 44087674, 44087769-44091607, 44091691-44095982, 44096097-44101320, |
| 19 | MAPT | NM_005910 | chr17 | 43972053-44039685, 44039837-44049223, 44049312-44051749, 44051838-44055739, 44055807- |
| | | | | 44064404, 44064462-44068824, 44068953-44073763, 44074031-44087674, 44087769-44091607, 44091691- |
| | | | | 44095982, 44096097-44101320, |
| 19 | MAPT | NM_016841 | chr17 | 43972053-44039685, 44039837-44055739, 44055807-44064404, 44064462-44068824, 44068953- |
| | | | | 44073763, 44074031-44091607, 44091691-44095982, 44096097-44101320, |
| 19 | MAPT | NM_016834 | chr17 | 43972053-44039685, 44039837-44055739, 44055807-44064404, 44064462-44068824, 44068953- |
| | | | | 44073763, 44074031-44087674, 44087769-44091607, 44091691-44095982, 44096097-44101320, |
| 19 | NFE2L1 | NM_003204 | chr17 | 46125825-46127967, 46128991-46133746, 46133961-46134392, 46134484-46134704, 46134865-46135655, |
| 20 | LRP11 | NM_032832 | chr6 | 150141840-150147398, 150147496-150157219, 150157434-150158472, 150158600- |
| | | | | 150164117, 150164261-150174137, 150174297-150184542, |
| 20 | ULBP1 | NM_025218 | chr6 | 150285271-150289741, 150290007-150290219, 150290497-150291150, 150291284-150292503, |
| 20 | ULBP3 | NM_024518 | chr6 | 150385850-150386532, 150386810-150387033, 150387299-150390113, |
| 20 | PPP1R14C | NM_030949 | chr6 | 150464635-150535878, 150535964-150537958, 150537993-150569880, |
| 20 | PLEKHG1 | NM_001029884 | chr6 | 150921154-150971862, 150971923-151054718, 151055229-151089772, 151089875- |
| | | | | 151107524, 151107596-151116990, 151117039-151121853, 151122006-151125744, 151125878- |
| | | | | 151130239, 151130424-151130515, 151130613-151130782, 151130871-151139317, 151139353- |
| | | | | 151140813, 151140911-151142329, 151142370-151144788, 151144813-151151716, 151153342- |
| | | | | 151160967, |
| 20 | FBXO5 | NM_012177 | chr6 | 153292550-153293405, 153293590-153294179, 153294272-153296040, 153296757-153303990, |
| 20 | FBXO5 | NM_001142522 | chr6 | 153292550-153293405, 153293590-153294179, 153294272-153296040, 153296757-153304367, |
| 20 | EZR | NM_001111077 | chr6 | 159188111-159188291, 159188545-159190356, 159190451-159190814, 159190977- |
| | | | | 159191794, 159191927-159192274, 159192440-159197438, 159197537-159204550, 159204699- |
| | | | | 159205674, 159205760-159206339, 159206616-159208138, 159208236-159210318, 159210404- |
| | | | | 159239112, 159239199-159240347, |
| 20 | EZR | NM_003379 | chr6 | 159188111-159188291, 159188545-159190356, 159190451-159190814, 159190977- |
| | | | | 159191794, 159191927-159192274, 159192440-159197438, 159197537-159204550, 159204699- |
| | | | | 159205674, 159205760-159206339, 159206616-159208138, 159208236-159210318, 159210404- |
| | | | | 159239112, |
| 20 | LOC100129518 | NR_037166 | chr6 | 160182669-160183024, |
| 20 | IGF2R | NM_000876 | chr6 | 160390428-160412214, 160412356-160430040, 160430167-160431717, 160431818- |
| | | | | 160445602, 160445737-160448215, 160448347-160450580, 160450688-160453581, 160453746- |
| | | | | 160453972, 160454140-160455449, 160455555-160461590, 160461757-160464178, 160464321- |
| | | | | 160465544, 160465690-160466775, 160466915-160467528, 160467678-160468189, 160468369- |
| | | | | 160468822, 160468940-160469405, 160469576-160471503, 160471685-160477454, 160477558- |
| | | | | 160479053, 160479157-160479936, 160480131-160481577, 160481750-160482533, 160482679- |
| | | | | 160482783, 160482961-160483562, 160483652-160484445, 160484663-160485431, 160485564- |
| | | | | 160485834, 160485934-160489279, 160489418-160490898, 160491091-160492940, 160493069- |
| | | | | 160493795, 160493917-160494243, 160494502-160494787, 160495008-160496877, 160497029- |
| | | | | 160499231, 160499395-160500610, 160500820-160501159, 160501308-160504980, 160505217- |
| | | | | 160506025, 160506164-160509063, 160509180-160510137, 160510286-160510946, 160511136- |
| | | | | 160517469, 160517658-160523549, 160523704-160524776, 160524848-160525704, |
| 20 | MAP3K4 | NM_006724 | chr6 | 161413116-161455289, 161455482-161469646, 161471012-161491638, 161491883- |
| | | | | 161494496, 161494645-161501911, 161502071-161505531, 161505650-161507409, 161507511- |
| | | | | 161507614, 161507700-161508718, 161508987-161510352, 161510504-161512409, 161512573- |
| | | | | 161513040, 161513176-161514008, 161514089-161514803, 161514884-161518110, 161518209- |
| | | | | 161522922, 161523025-161523729, 161523812-161527544, 161527738-161528929, 161529002- |
| | | | | 161529773, 161529892-161530785, 161530947-161532866, 161532991-161533698, 161533807- |
| | | | | 161536153, 161536335-161537880, |
| 20 | MAP3K4 | NM_005922 | chr6 | 161413116-161455289, 161455482-161469646, 161471012-161491638, 161491883- |
| | | | | 161494496, 161494645-161501911, 161502071-161505531, 161505650-161507409, 161507511- |
| | | | | 161507614, 161507700-161508718, 161508987-161510352, 161510504-161512409, 161512573- |
| | | | | 161513040, 161513176-161514008, 161514089-161514803, 161514884-161518110, 161518209- |
| | | | | 161519308, 161519460-161522922, 161523025-161523729, 161523812-161527544, 161527738- |
| | | | | 161528929, 161529002-161529773, 161529892-161530785, 161530947-161532866, 161532991- |
| | | | | 161533698, 161533807-161536153, 161536335-161537880, |
| 20 | TTLL2 | NM_031949 | chr6 | 167738709-167752133, 167752292-167753591, |
| 20 | C6orf124 | NR_027906 | chr6 | 168225487-168226081, |
| 21 | POLQ | NM_199420 | chr3 | 121151265-121151763, 121151881-121154967, 121155123-121158837, 121158964- |
| | | | | 121168160, 121168274-121178895, 121179082-121186364, 121186488-121187150, 121187279- |
| | | | | 121190835, 121191012-121192195, 121192335-121195385, 121195581-121200417, 121200660- |
| | | | | 121202231, 121202430-121203886, 121204032-121206147, 121209256-121212323, 121212569- |
| | | | | 121215653, 121215780-121217322, 121217518-121228406, 121228551-121228884, 121229091- |
| | | | | 121230732, 121230877-121238716, 121238931-121240848, 121240997-121248490, 121248640- |
| | | | | 121251835, 121252057-121255945, 121256056-121258278, 121258437-121260194, 121260327- |
| | | | | 121263572, 121263754-121264560, |
| 21 | PDIA5 | NM_006810 | chr3 | 122786054-122808013, 122808142-122811200, 122811290-122821256, 122821342- |
| | | | | 122821596, 122821644-122825594, 122825689-122829789, 122829852-122835076, 122835146- |
| | | | | 122842911, 122843005-122843117, 122843191-122849325, 122849464-122864367, 122864440- |
| | | | | 122864944, 122865107-122869079, 122869212-122873816, 122873889-122880166, 122880303- |
| | | | | 122880725, |
| 21 | PDIA5 | NR_028444 | chr3 | 122786054-122808013, 122808142-122811200, 122811290-122821256, 122821342- |
| | | | | 122821596, 122821644-122825594, 122825689-122829789, 122829852-122835076, 122835146- |
| | | | | 122842911, 122843005-122843117, 122843191-122864367, 122864440-122864963, 122865107- |
| | | | | 122869079, 122869212-122873816, 122873889-122880166, 122880303-122880725, |
| 21 | PTPLB | NM_198402 | chr3 | 123213818-123219360, 123219541-123221405, 123221529-123247231, 123247322- |
| | | | | 123286607, 123286628-123301057, 123301177-123303718, |
| 21 | ALG1L | NM_001015050 | chr3 | 125648411-125649398, 125649473-125650479, 125650596-125651491, 125651553- |
| | | | | 125652416, 125652540-125655739, |
| 21 | ALG1L | NM_001195223 | chr3 | 125648411-125649398, 125649473-125650479, 125650596-125651491, 125651553- |
| | | | | 125652416, 125652540-125653277, 125653390-125655739, |
| 21 | PODXL2 | NM_015720 | chr3 | 127348133-127358086, 127358367-127379219, 127380003-127381078, 127381155- |
| | | | | 127387282, 127387441-127387982, 127388046-127390275, 127390457-127391109, |
| 21 | GP9 | NM_000174 | chr3 | 128779694-128780335, 128780463-128780569, |
| 22 | CIC | NM_015125 | chr19 | 42788924-42790921, 42791073-42791156, 42791393-42791470, 42791602-42791695, 42791880- |
| | | | | 42791960, 42792128-42793038, 42793243-42793331, 42793559-42793998, 42794104-42794383, 42795619- |
| | | | | 42795708, 42795898-42796236, 42796360-42796450, 42796619-42796716, 42797012-42797106, 42797434- |
| | | | | 42797742, 42797989-42798085, 42798242-42798323, 42798457-42798754, 42798888-42798974, |
| 22 | TEX101 | NM_001130011 | chr19 | 43919191-43920031, 43920136-43920249, 43920395-43920523, 43920708-43922028, 43922159-43922318, |
| 22 | TEX101 | NM_031451 | chr19 | 43892882-43905646, 43905714-43906916, 43907012-43910373, 43910672-43920031, 43920136- |
| | | | | 43920249, 43920395-43920523, 43920708-43922028, 43922159-43922318, |
| 22 | CADM4 | NM_145296 | chr19 | 44127592-44128264, 44128395-44129228, 44129403-44130061, 44130154-44130274, 44130441- |
| | | | | 44130934, 44131071-44131316, 44131471-44131794, 44131943-44143877, |
| 22 | ZNF223 | NM_013361 | chr19 | 44556349-44559260, 44559345-44564606, 44564735-44564900, 44564995-44570215, |
| 22 | ZNF284 | NM_001037813 | chr19 | 44576445-44580473, 44580558-44585166, 44585295-44586164, 44586259-44589865, |
| 22 | LOC100379224 | NR_033341 | chr19 | 44612491-44616973, |
| 22 | ZFP112 | NM_013380 | chr19 | 44834090-44840774, 44840872-44844566, 44844695-44860776, |
| 22 | ZFP112 | NM_001083335 | chr19 | 44834090-44840774, 44840872-44844566, 44844695-44847404, 44847455-44860776, |
| 22 | CLPTM1 | NM_001199468 | chr19 | 45458725-45465218, 45465333-45476342, 45476468-45477694, 45477855-45480598, 45480718- |
| | | | | 45488474, 45488562-45489711, 45489834-45490435, 45490682-45491336, 45491432-45493651, 45493844- |
| | | | | 45494106, 45494204-45494494, 45494632-45495489, 45495659-45495867, 45496114-45496323, |
| 22 | CLPTM1 | NM_001294 | chr19 | 45458725-45465218, 45465333-45476342, 45476468-45477694, 45477855-45480598, 45480718- |
| | | | | 45488474, 45488562-45489711, 45489834-45490435, 45490682-45491336, 45491432-45493651, 45493844- |
| | | | | 45494106, 45494204-45494494, 45494632-45495489, 45495659-45495867, |
| 22 | CLASRP | NM_007056 | chr19 | 45542367-45543440, 45543570-45555327, 45555427-45556047, 45556151-45556354, 45556436- |
| | | | | 45559706, 45559793-45561006, 45561157-45562524, 45562623-45563645, 45563726-45563810, 45563886- |
| | | | | 45565410, 45565454-45567268, 45567486-45567599, 45567889-45570593, 45570853-45571271, 45571313- |
| | | | | 45571676, 45571739-45572322, 45572383-45572458, 45572507-45573278, 45573363-45573793, 45573820- |
| | | | | 45574059, |
| 22 | TRAPPC6A | NM_024108 | chr19 | 45666482-45667172, 45667268-45667420, 45667506-45668109, 45668229-45668383, 45668453-45681349, |
| 22 | SIX5 | NM_175875 | chr19 | 46269370-46269606, 46270414-46271298, |
| 22 | DMWD | NM_004943 | chr19 | 46287549-46287897, 46287974-46288850, 46290130-46294161, 46294346-46295572, |
| 22 | MEIS3 | NM_001009813 | chr19 | 47906811-47909715, 47909784-47910143, 47910229-47910334, 47910395-47910607, 47910686- |
| | | | | 47912354, 47912505-47912691, 47912805-47917972, 47918073-47918306, 47918359-47919907, 47919960- |
| | | | | 47920059, 47920221-47920433, 47920608-47922325, |
| 22 | MEIS3 | NM_020160 | chr19 | 47906811-47909715, 47909784-47910143, 47910229-47910334, 47910395-47910607, 47910824- |
| | | | | 47912354, 47912505-47912691, 47912805-47917972, 47918124-47918306, 47918359-47919907, 47919960- |
| | | | | 47920059, 47920221-47920433, 47920608-47922325, |
| 22 | EMP3 | NM_001425 | chr19 | 48828868-48830085, 48830180-48830776, 48830881-48832607, 48832750-48833556, |
| 22 | CYTH2 | NM_017457 | chr19 | 48972784-48973608, 48973758-48973858, 48973927-48975580, 48975701-48976553, 48976636- |
| | | | | 48977160, 48977275-48977437, 48977588-48978093, 48978208-48981322, 48981403-48981530, 48981604- |
| | | | | 48981693, 48981850-48982378, |
| 22 | CYTH2 | NM_004228 | chr19 | 48972784-48973608, 48973758-48973858, 48973927-48975580, 48975701-48976553, 48976636- |
| | | | | 48977160, 48977275-48977437, 48977588-48978093, 48978207-48981324, 48981403-48981530, 48981604- |
| | | | | 48981693, 48981850-48982378, |
| 23 | SARNP | NR_026722 | chr12 | 56146625-56151165, 56151338-56154333, 56154425-56182857, 56182928-56184349, 56184377- |
| | | | | 56188141, 56188172-56188589, 56188665-56189807, 56189861-56194344, 56194414-56197411, 56197513- |
| | | | | 56211448, |
| 23 | SARNP | NM_033082 | chr12 | 56151338-56154333, 56154425-56182857, 56182928-56184349, 56184377-56188141, 56188172- |
| | | | | 56188589, 56188665-56189807, 56189861-56194344, 56194414-56194636, 56194685-56197411, 56197513- |
| | | | | 56211448, |
| 23 | SARNP | NR_026723 | chr12 | 56146625-56151165, 56151338-56154333, 56154425-56182857, 56182928-56184349, 56184377- |
| | | | | 56188141, 56188172-56188589, 56188665-56189807, 56189861-56194344, 56194414-56194636, 56194685- |
| | | | | 56197411, 56197513-56211448, |
| 23 | APOF | NM_001638 | chr12 | 56755974-56756512, |
| 23 | MIP | NM_012064 | chr12 | 56845250-56846854, 56846937-56847373, 56847540-56848036, |
| 23 | NXPH4 | NM_007224 | chr12 | 57610810-57618659, |
| 23 | INHBE | NM_031479 | chr12 | 57849618-57849875, |
| 23 | LRIG3 | NM_001136051 | chr12 | 59266599-59267835, 59268113-59268210, 59268356-59270225, 59270386-59271180, 59271635- |
| | | | | 59272604, 59272888-59274361, 59274684-59276649, 59276815-59277300, 59277374-59279611, 59279685- |
| | | | | 59280590, 59280673-59281569, 59281715-59282109, 59282255-59282599, 59282745-59283776, 59283922- |
| | | | | 59284445, 59284579-59307761, 59307838-59308044, 59308118-59313206, |
| 23 | LRIG3 | NM_153377 | chr12 | 59266599-59267835, 59268113-59268210, 59268356-59270225, 59270386-59271180, 59271635- |
| | | | | 59272604, 59272888-59274361, 59274684-59276649, 59276815-59277300, 59277374-59279611, 59279685- |
| | | | | 59280590, 59280673-59281569, 59281715-59282109, 59282255-59282599, 59282745-59283776, 59283922- |
| | | | | 59284445, 59284579-59307761, 59307838-59308044, 59308118-59313779, |
| 24 | ZNF175 | NM_007147 | chr19 | 52074729-52076401, 52076655-52084642, 52084771-52085038, 52085136-52089878, |
| 24 | ZNF616 | NM_178523 | chr19 | 52620278-52627174, 52627303-52633752, 52633842-52642983, |
| 24 | ZNF836 | NM_001102657 | chr19 | 52660794-52663716, 52663845-52671309, 52671406-52672899, 52673035-52674587, |
| 24 | ZNF610 | NM_001161427 | chr19 | 52839760-52850958, 52851198-52852405, 52852489-52856933, 52857062-52868949, |
| 24 | ZNF610 | NM_001161425 | chr19 | 52839760-52850958, 52851198-52852405, 52852489-52856933, 52857062-52857502, 52857633-52868949, |
| 24 | ZNF610 | NM_001161426 | chr19 | 52839760-52850958, 52851085-52852405, 52852489-52856933, 52857062-52857502, 52857633-52868949, |
| 24 | ZNF610 | NM_173530 | chr19 | 52848800-52850958, 52851198-52852405, 52852489-52856933, 52857062-52857502, 52857633-52868949, |
| 24 | ZNF528 | NM_032423 | chr19 | 52901158-52901618, 52901873-52904936, 52905007-52905198, 52905282-52909158, 52909287- |
| | | | | 52909766, 52909897-52918375, |
| 24 | ZNF578 | NM_001099694 | chr19 | 52956861-52960119, 52960212-52994575, 52994679-53005078, 53005162-53007906, 53008035-53013823, |
| 24 | ZNF137P | NR_023311 | chr19 | 53100824-53101466, 53102059-53102398, |
| 25 | C17orf102 | NM_207454 | chr17 | 32904640-32905888, |
| 25 | LYZL6 | NM_020426 | chr17 | 34261870-34263757, 34263838-34264760, 34264921-34266220, |
| 25 | LYZL6 | NM_001199951 | chr17 | 34261870-34263757, 34263838-34264760, 34264921-34266220, 34266449-34270569, |
| 25 | LHX1 | NM_005568 | chr17 | 35295665-35297585, 35297814-35297905, 35298185-35299495, 35299663-35300047, |
| 25 | NEUROD2 | NM_006160 | chr17 | 37762858-37763979, |
| 25 | ZPBP2 | NM_199321 | chr17 | 38024660-38024800, 38024868-38026945, 38027073-38027715, 38027879-38028521, 38028742- |
| | | | | 38029295, 38029380-38031505, 38031688-38032933, |
| 25 | ZPBP2 | NM_198844 | chr17 | 38024660-38026945, 38027073-38027715, 38027879-38028521, 38028742-38029295, 38029380- |
| | | | | 38031505, 38031688-38032933, |
| 25 | KRT27 | NM_181537 | chr17 | 38933391-38933503, 38933555-38933765, 38933985-38935752, 38935880-38935950, 38936114- |
| | | | | 38936550, 38936709-38937438, 38937523-38938300, |
| 25 | KRT16 | NM_005557 | chr17 | 39766282-39766440, 39766489-39766581, 39766804-39767104, 39767232-39767319, 39767483- |
| | | | | 39767595, 39767754-39767889, 39767974-39768408, |
| 26 | TMEM132A | NM_178031 | chr11 | 60692206-60694674, 60694891-60695111, 60695332-60696099, 60696433-60697980, 60698132- |
| | | | | 60699159, 60699357-60699454, 60699600-60701012, 60701217-60701958, 60702237-60702722, 60702916- |
| | | | | 60703334, |
| 26 | TMEM132A | NM_017870 | chr11 | 60692206-60694674, 60694891-60695111, 60695332-60696099, 60696433-60697980, 60698132- |
| | | | | 60699156, 60699357-60699454, 60699600-60701012, 60701217-60701958, 60702237-60702722, 60702916- |
| | | | | 60703334, |
| 26 | PGA3 | NM_001079807 | chr11 | 60971093-60971577, 60971742-60973980, 60974100-60974967, 60975088-60975761, 60975963- |
| | | | | 60977312, 60977431-60978560, 60978707-60978807, 60978908-60980023, |
| 26 | FADS2 | NM_004265 | chr11 | 61596070-61605248, 61605361-61607804, 61608004-61608094, 61608198-61615629, 61615757- |
| | | | | 61624481, 61624544-61624924, 61625003-61630442, 61630542-61630752, 61630851-61631177, 61631259- |
| | | | | 61632622, 61632750-61633108, |
| 26 | INCENP | NM_001040694 | chr11 | 61891636-61895621, 61895774-61896240, 61896356-61897252, 61898063-61905302, 61905356- |
| | | | | 61906183, 61906243-61906358, 61906478-61907704, 61907758-61908174, 61908294-61908383, 61908517- |
| | | | | 61908970, 61908984-61912452, 61912564-61912639, 61912762-61913099, 61913224-61914128, 61914375- |
| | | | | 61915946, 61916027-61916209, 61916319-61917527, 61917680-61919232, |
| 26 | INCENP | NM_020238 | chr11 | 61891636-61895621, 61895774-61896240, 61896356-61897252, 61898063-61905302, 61905356- |
| | | | | 61906183, 61906243-61906358, 61906478-61907704, 61907758-61908174, 61908294-61908383, 61908517- |
| | | | | 61912452, 61912564-61912639, 61912762-61913099, 61913224-61914128, 61914375-61915946, 61916027- |
| | | | | 61916209, 61916319-61917527, 61917680-61919232, |
| 26 | SLC3A2 | NM_002394 | chr11 | 62623854-62638215, 62638310-62639047, 62639142-62648489, 62648920-62649363, 62649539- |
| | | | | 62650378, 62650472-62651927, 62651998-62652102, 62652163-62652647, 62652830-62652935, 62653081- |
| | | | | 62655605, 62655691-62655801, |
| 26 | SLC3A2 | NM_001012662 | chr11 | 62623854-62639047, 62639142-62644254, 62644352-62648489, 62648920-62649363, 62649539- |
| | | | | 62650378, 62650472-62651927, 62651998-62652102, 62652163-62652647, 62652830-62652935, 62653081- |
| | | | | 62655605, 62655691-62655801, |
| 26 | SLC3A2 | NM_001012664 | chr11 | 62623854-62648489, 62648920-62649363, 62649539-62650378, 62650472-62651927, 62651998- |
| | | | | 62652102, 62652163-62652647, 62652830-62652935, 62653081-62655605, 62655691-62655801, |
| 26 | SLC3A2 | NM_001012663 | chr11 | 62623854-62639047, 62639142-62648489, 62648920-62649363, 62649539-62650378, 62650472- |
| | | | | 62651927, 62651998-62652102, 62652163-62652647, 62652830-62652935, 62653081-62655605, 62655691- |
| | | | | 62655801, |
| 26 | SLC3A2 | NM_001013251 | chr11 | 62648920-62649363, 62649539-62650378, 62650472-62651927, 62651998-62652102, 62652163- |
| | | | | 62652647, 62652830-62652935, 62653081-62655605, 62655691-62655801, |
| 26 | SLC3A2 | NM_001012661 | chr11 | 62623854-62638215, 62638310-62639047, 62639142-62644257, 62644352-62648489, 62648920- |
| | | | | 62649363, 62649539-62650378, 62650472-62651927, 62651998-62652102, 62652163-62652647, 62652830- |
| | | | | 62652935, 62653081-62655605, 62655691-62655801, |
| 26 | SLC3A2 | NR_037193 | chr11 | 62649539-62650378, 62650472-62651459, 62651585-62651927, 62651998-62652102, 62652163- |
| | | | | 62652647, 62652830-62652935, 62653081-62655605, 62655691-62655801, |
| 26 | SLC22A6 | NM_004790 | chr11 | 62744354-62744615, 62744860-62746958, 62747069-62747204, 62747421-62748455, 62748573- |
| | | | | 62748730, 62748856-62749312, 62749483-62751007, 62751164-62751414, 62751520-62751792, |
| 26 | SLC22A6 | NM_153276 | chr11 | 62744354-62744654, 62744860-62746958, 62747069-62747204, 62747421-62748455, 62748573- |
| | | | | 62748730, 62748856-62749312, 62749483-62751007, 62751164-62751414, 62751520-62751792, |
| 26 | SLC22A6 | NM_153277 | chr11 | 62744354-62744654, 62744728-62746958, 62747069-62747204, 62747421-62748455, 62748573- |
| | | | | 62748730, 62748856-62749312, 62749483-62751007, 62751164-62751414, 62751520-62751792, |
| 26 | SLC22A6 | NM_153278 | chr11 | 62744354-62744615, 62744728-62746958, 62747069-62747204, 62747421-62748455, 62748573- |
| | | | | 62748730, 62748856-62749312, 62749483-62751007, 62751164-62751414, 62751520-62751792, |
| 27 | RRP8 | NM_015324 | chr11 | 6621496-6621714, 6621813-6621897, 6622006-6622154, 6622286-6622377, 6622833-6623080, 6623446- |
| | | | | 6624632, |
| 27 | DCHS1 | NM_003737 | chr11 | 6645622-6645959, 6646100-6646427, 6646713-6646826, 6646959-6647149, 6647306-6647398, 6647612- |
| | | | | 6647781, 6647897-6648018, 6648899-6649850, 6650062-6650681, 6650809-6650901, 6651143- |
| | | | | 6651228, 6652095-6652282, 6652442-6652540, 6652639-6652845, 6653041-6653260, 6654288- |
| | | | | 6654641, 6654880-6655018, 6655252-6655347, 6655538-6661046, 6662965-6676792, |
| 27 | ZNF214 | NM_013249 | chr11 | 7022787-7023938, 7024087-7041302, |
| 27 | SYT9 | NM_175733 | chr11 | 7273563-7324268, 7324622-7334624, 7335173-7437271, 7437394-7439186, 7439360-7441735, 7441867- |
| | | | | 7488021, |
| 27 | TRIM66 | NM_014818 | chr11 | 8639551-8640295, 8640516-8640928, 8641083-8641595, 8641672-8641991, 8642120-8642577, 8642869- |
| | | | | 8643190, 8643367-8644361, 8644424-8645904, 8646002-8646254, 8646776-8660199, 8660363- |
| | | | | 8661772, 8662700-8664554, 8664674-8667286, 8667435-8667992, 8668109-8669515, 8669634- |
| | | | | 8669961, 8670096-8671285, 8671435-8673345, 8673451-8680282, |
| 27 | NRIP3 | NM_020645 | chr11 | 9005108-9005420, 9005517-9005617, 9005672-9007256, 9007398-9009093, 9009178-9009663, 9009830- |
| | | | | 9025307, |
| 27 | SCUBE2 | NM_001170690 | chr11 | 9042745-9043421, 9043503-9047247, 9047402-9051429, 9051593-9052303, 9052473-9055171, 9055344- |
| | | | | 9068901, 9069110-9075182, 9075307-9077338, 9077457-9080848, 9080973-9081953, 9082072- |
| | | | | 9087436, 9087528-9088242, 9088361-9090915, 9091043-9096026, 9096163-9100929, 9101057- |
| | | | | 9111252, 9111377-9112941, |
| 27 | SCUBE2 | NM_020974 | chr11 | 9042745-9043421, 9043503-9047247, 9047402-9048909, 9049109-9051429, 9051593-9052303, 9052473- |
| | | | | 9068901, 9069110-9069488, 9069646-9072151, 9072258-9074291, 9074380-9074644, 9074763- |
| | | | | 9075182, 9075307-9077338, 9077457-9080848, 9080973-9081953, 9082072-9087436, 9087528- |
| | | | | 9088242, 9088361-9090915, 9091043-9096026, 9096163-9100929, 9101057-9111252, 9111377-9112941, |
| 28 | C17orf97 | NM_001013672 | chr17 | 260438-262937, |
| 28 | RNMTL1 | NM_018146 | chr17 | 685933-686321, 686568-691203, 691373-694772, |
| 28 | ABR | NM_001092 | chr17 | 909410-910403, 910553-912917, 913025-913967, 914104-915084, 915226-915926, 916038-916343, 916405- |
| | | | | 953288, 953422-953775, 953875-959273, 959350-960236, 960343-961208, 961286-961983, 962108- |
| | | | | 970315, 970483-973207, 973331-975852, 975995-976863, 976918-982568, 982631-986758, 986868- |
| | | | | 994903, 995091-1003875, 1003976-1012172, |
| 28 | ABR | NM_001159746 | chr17 | 909410-910403, 910553-912917, 913025-913967, 914104-915084, 915226-915926, 916038-916343, 916405- |
| | | | | 953288, 953422-953775, 953875-959273, 959350-960236, 960343-961208, 961286-961983, 962108- |
| | | | | 970315, 970483-973207, 973331-975852, 975995-976863, 976918-982568, 982631-986758, 986868- |
| | | | | 994903, 995091-1003875, 1003976-1028516, 1028703-1090092, |
| 28 | ABR | NM_021962 | chr17 | 909410-910403, 910553-912917, 913025-913967, 914104-915084, 915226-915926, 916038-916343, 916405- |
| | | | | 953288, 953422-953775, 953875-959273, 959350-960236, 960343-961208, 961286-961983, 962108- |
| | | | | 970315, 970483-973207, 973331-975852, 975995-976863, 976918-982568, 982631-986758, 986868- |
| | | | | 994903, 995091-1003875, 1003976-1028516, 1028703-1082959, |
| 28 | YWHAE | NM_006761 | chr17 | 1248794-1257503, 1257642-1264384, 1264593-1265194, 1265303-1268151, 1268353-1303339, |
| 28 | YWHAE | NR_024058 | chr17 | 1248794-1257503, 1257642-1264384, 1264593-1265194, 1265303-1268151, 1268353-1273001, 1273036- |
| | | | | 1303339, |
| 28 | MYO1C | NM_033375 | chr17 | 1369047-1370534, 1370636-1370772, 1370872-1371132, 1371205-1371280, 1371418-1371545, 1371603- |
| | | | | 1371695, 1371791-1372823, 1372909-1373467, 1373629-1373727, 1373814-1373913, 1373984- |
| | | | | 1374363, 1374442-1374515, 1374631-1375199, 1375319-1375415, 1375523-1377902, 1377985- |
| | | | | 1378104, 1378153-1378230, 1378327-1380797, 1380891-1381183, 1381266-1381379, 1381487- |
| | | | | 1381702, 1381787-1381908, 1382030-1382723, 1382797-1382884, 1383000-1383819, 1383920- |
| | | | | 1383998, 1384180-1385770, 1385853-1386153, 1386354-1386902, 1387020-1387440, 1387598-1394892, |
| 28 | MYO1C | NM_001080950 | chr17 | 1369047-1370534, 1370636-1370772, 1370872-1371132, 1371205-1371280, 1371418-1371545, 1371603- |
| | | | | 1371695, 1371791-1372823, 1372909-1373467, 1373629-1373727, 1373814-1373913, 1373984- |
| | | | | 1374363, 1374442-1374515, 1374631-1375199, 1375319-1375415, 1375523-1377902, 1377985- |
| | | | | 1378104, 1378153-1378230, 1378327-1380797, 1380891-1381183, 1381266-1381379, 1381487- |
| | | | | 1381702, 1381787-1381908, 1382030-1382723, 1382797-1382884, 1383000-1383819, 1383920- |
| | | | | 1383998, 1384180-1385770, 1385853-1386153, 1386354-1386902, 1387020-1387440, 1387598-1388986, |
| 28 | MYO1C | NM_001080779 | chr17 | 1369047-1370534, 1370636-1370772, 1370872-1371132, 1371205-1371280, 1371418-1371545, 1371603- |
| | | | | 1371695, 1371791-1372823, 1372909-1373467, 1373629-1373727, 1373814-1373913, 1373984- |
| | | | | 1374363, 1374442-1374515, 1374631-1375199, 1375319-1375415, 1375523-1377902, 1377985- |
| | | | | 1378104, 1378153-1378230, 1378327-1380797, 1380891-1381183, 1381266-1381379, 1381487- |
| | | | | 1381702, 1381787-1381908, 1382030-1382723, 1382797-1382884, 1383000-1383819, 1383920- |
| | | | | 1383998, 1384180-1385770, 1385853-1386153, 1386354-1386902, 1387020-1387440, 1387598-1395705, |
| 28 | INPP5K | NM_001135642 | chr17 | 1399174-1399352, 1399459-1399611, 1399697-1399958, 1400098-1401228, 1401417-1410272, 1410384- |
| | | | | 1411407, 1411521-1412470, 1412648-1412975, 1413094-1416745, 1416856-1417164, 1417274- |
| | | | | 1418719, 1418880-1419180, 1419413-1419748, |
| 28 | INPP5K | NM_130766 | chr17 | 1399174-1399352, 1399459-1399611, 1399697-1399958, 1400098-1401228, 1401417-1410272, 1410384- |
| | | | | 1411407, 1411521-1412470, 1412648-1412975, 1413094-1416745, 1416856-1417164, 1417274- |
| | | | | 1419180, 1419413-1419748, |
| 28 | INPP5K | NM_016532 | chr17 | 1399174-1399352, 1399459-1399611, 1399697-1399958, 1400098-1401228, 1401417-1410272, 1410384- |
| | | | | 1411407, 1411521-1412470, 1412648-1412975, 1413094-1416745, 1416856-1417164, 1417274-1419748, |
| 28 | SCARF1 | NM_145352 | chr17 | 1538912-1540001, 1540150-1540233, 1540357-1542098, 1542221-1543765, 1543961-1546734, 1547262- |
| | | | | 1548126, 1548230-1548470, 1548534-1548889, |
| 28 | SCARF1 | NM_145350 | chr17 | 1538912-1540001, 1540113-1540233, 1540357-1542098, 1542221-1542931, 1543037-1543204, 1543335- |
| | | | | 1543740, 1543961-1546734, 1547262-1548126, 1548230-1548470, 1548534-1548889, |
| 28 | SCARF1 | NM_003693 | chr17 | 1538912-1540001, 1540150-1540233, 1540357-1542098, 1542221-1542931, 1543037-1543204, 1543335- |
| | | | | 1543740, 1543961-1546734, 1547262-1548126, 1548230-1548470, 1548534-1548889, |
| 28 | SCARF1 | NR_028076 | chr17 | 1538912-1540001, 1540150-1540233, 1540357-1542098, 1542221-1542931, 1543037-1543204, 1543961- |
| | | | | 1546734, 1547262-1548126, 1548230-1548470, 1548534-1548889, |
| 28 | SCARF1 | NR_028075 | chr17 | 1538912-1540001, 1540150-1540233, 1540357-1542098, 1542221-1542931, 1543037-1543204, 1543265- |
| | | | | 1543740, 1543961-1546734, 1547262-1548126, 1548230-1548470, 1548534-1548889, |
| 28 | PRPF8 | NM_006445 | chr17 | 1554251-1554400, 1554605-1554706, 1554848-1554940, 1555083-1556834, 1556978-1557069, 1557311- |
| | | | | 1558642, 1558838-1559684, 1559860-1559940, 1560056-1561545, 1561676-1561818, 1562058- |
| | | | | 1562649, 1562843-1563133, 1563296-1563724, 1563873-1563990, 1564122-1564285, 1564457- |
| | | | | 1564563, 1564701-1564903, 1565085-1565198, 1565448-1576373, 1576492-1576649, 1576862- |
| | | | | 1577038, 1577187-1577734, 1577975-1578444, 1578634-1578912, 1579107-1579220, 1579349- |
| | | | | 1579499, 1579665-1579797, 1580006-1580268, 1580467-1580857, 1580989-1581810, 1581947- |
| | | | | 1582054, 1582176-1582309, 1582501-1582583, 1582705-1582901, 1583094-1584018, 1584126- |
| | | | | 1584221, 1584349-1584770, 1584985-1585112, 1585333-1585421, 1585588-1586825, 1586996- |
| | | | | 1587764, 1587877-1588072, |
| 28 | SERPINF1 | NM_002615 | chr17 | 1665408-1670195, 1670289-1673144, 1673345-1674321, 1674479-1675164, 1675370-1678350, 1678495- |
| | | | | 1679824, 1680037-1680479, |
| 28 | RTN4RL1 | NM_178568 | chr17 | 1841103-1928145, |
| 28 | SMG6 | NM_017575 | chr17 | 1964917-1968365, 1968449-1968760, 1968974-1972070, 1972226-1985102, 1985251-1989017, 1989196- |
| | | | | 2075950, 2076154-2089955, 2090126-2091691, 2091810-2139784, 2139932-2147950, 2148014- |
| | | | | 2185939, 2186154-2186917, 2187030-2195844, 2195925-2196163, 2196272-2200535, 2200648- |
| | | | | 2201155, 2201350-2202198, 2203959-2206925, |
| 28 | SMG6 | NM_001170957 | chr17 | 1964917-1968365, 1968449-1968760, 1968974-1972070, 1972226-1985102, 1985251-1989017, 1989196- |
| | | | | 2075950, 2076154-2089955, 2090126-2091691, 2091810-2139784, 2139932-2147950, 2148014- |
| | | | | 2185939, 2186154-2186917, 2187030-2195844, 2195925-2196163, 2196272-2200535, 2200648- |
| | | | | 2201155, 2201350-2202198, 2203959-2206306, |
| 28 | SGSM2 | NM_014853 | chr17 | 2241040-2246837, 2246915-2264929, 2265094-2265401, 2265565-2266130, 2266200-2266281, 2266429- |
| | | | | 2266757, 2266894-2267351, 2267478-2267888, 2267958-2268189, 2268352-2268507, 2268636- |
| | | | | 2270563, 2270700-2274554, 2274710-2275482, 2275549-2275635, 2275783-2276245, 2276376- |
| | | | | 2276623, 2276808-2278784, 2279170-2279429, 2279556-2280025, 2280110-2280251, 2280363- |
| | | | | 2281149, 2281248-2282328, 2282498-2282724, |
| 28 | SGSM2 | NM_001098509 | chr17 | 2241040-2246837, 2246915-2264929, 2265094-2265401, 2265565-2266130, 2266200-2266281, 2266429- |
| | | | | 2266757, 2266894-2267351, 2267478-2267888, 2267958-2268189, 2268352-2268507, 2268636- |
| | | | | 2274554, 2274710-2275482, 2275549-2275635, 2275783-2276245, 2276376-2276623, 2276808- |
| | | | | 2278784, 2279170-2279429, 2279556-2280025, 2280110-2280251, 2280363-2281149, 2281248- |
| | | | | 2282328, 2282498-2282724, |
| 28 | TRPV1 | NM_080706 | chr17 | 3470282-3474816, 3474934-3475414, 3475544-3476925, 3477250-3480418, 3480487-3480890, 3481058- |
| | | | | 3483749, 3483822-3486630, 3486725-3489060, 3489221-3491480, 3491662-3493099, 3493400- |
| | | | | 3493544, 3493687-3494256, 3494411-3494479, 3494648-3495359, |
| 28 | TRPV1 | NM_080705 | chr17 | 3470282-3474816, 3474934-3475414, 3475544-3476925, 3477250-3480418, 3480487-3480890, 3481058- |
| | | | | 3483749, 3483822-3486630, 3486725-3489060, 3489221-3491480, 3491662-3493099, 3493400- |
| | | | | 3493544, 3493687-3494256, 3494411-3494479, 3494648-3495359, 3495678-3498956, |
| 28 | TRPV1 | NM_018727 | chr17 | 3470282-3474816, 3474934-3475414, 3475544-3476925, 3477250-3480418, 3480487-3480890, 3481058- |
| | | | | 3483749, 3483822-3486630, 3486725-3489060, 3489221-3491480, 3491662-3493099, 3493400- |
| | | | | 3493544, 3493687-3494256, 3494411-3494479, 3494648-3495359, 3495678-3500210, |
| 28 | TRPV1 | NM_080704 | chr17 | 3470282-3474816, 3474934-3475414, 3475544-3476925, 3477250-3480418, 3480487-3480890, 3481058- |
| | | | | 3483749, 3483822-3486630, 3486725-3489060, 3489221-3491480, 3491662-3493099, 3493400- |
| | | | | 3493544, 3493687-3494256, 3494411-3494479, 3494648-3495359, 3495678-3511719, 3511860-3512601, |
| 28 | C17orf85 | NM_001114118 | chr17 | 3716574-3717614, 3717778-3719408, 3719565-3721555, 3721867-3724541, 3724647-3725245, 3725347- |
| | | | | 3728234, 3728345-3729059, 3729138-3729379, 3729510-3732535, 3732663-3743392, 3743500- |
| | | | | 3746378, 3746446-3749333, |
| 28 | C17orf85 | NM_018553 | chr17 | 3716574-3717614, 3717778-3719408, 3719565-3721555, 3721867-3724541, 3724647-3725245, 3725347- |
| | | | | 3727352, 3727387-3728234, 3728345-3729059, 3729138-3729379, 3729510-3732535, 3732663- |
| | | | | 3743392, 3743500-3746378, 3746446-3749333, |
| 28 | ZZEF1 | NM_015113 | chr17 | 3910265-3912175, 3912249-3912896, 3913052-3916741, 3916909-3917382, 3917483-3917639, 3917810- |
| | | | | 3919615, 3919761-3920663, 3921025-3921128, 3921266-3922961, 3923064-3924421, 3924615- |
| | | | | 3926001, 3926123-3928211, 3928413-3935418, 3935553-3936120, 3936297-3937307, 3937587- |
| | | | | 3945721, 3945863-3947516, 3947669-3953000, 3953154-3954073, 3954338-3955263, 3955431- |
| | | | | 3957349, 3957490-3959508, 3959640-3961286, 3961450-3962463, 3962585-3966045, 3966212- |
| | | | | 3967653, 3968124-3969739, 3969835-3970455, 3970537-3973976, 3974219-3975900, 3976051- |
| | | | | 3977442, 3977646-3978389, 3978473-3978555, 3978724-3979929, 3980054-3980160, 3980284- |
| | | | | 3981175, 3981337-3984668, 3984785-3985729, 3985799-3988962, 3989098-3989778, 3989950- |
| | | | | 3990726, 3990829-3991970, 3992188-3994011, 3994125-3999123, 3999274-3999901, 3999995- |
| | | | | 4005609, 4005710-4007925, 4008106-4008985, 4009104-4012945, 4013158-4015901, 4016103- |
| | | | | 4017591, 4017765-4020264, 4020461-4027199, 4027346-4045834, |
| 28 | CYB5D2 | NR_023346 | chr17 | 4047058-4053183, 4053326-4057966, 4058155-4060158, |
| 28 | CYB5D2 | NR_023347 | chr17 | 4046608-4053183, 4053326-4057966, 4058155-4060158, |
| 28 | CYB5D2 | NM_144611 | chr17 | 4047300-4053183, 4053326-4057966, 4058155-4060158, |
| 28 | PLD2 | NM_002663 | chr17 | 4710521-4711065, 4711177-4711277, 4711410-4711567, 4711712-4712393, 4712501-4712591, 4712659- |
| | | | | 4712786, 4712847-4712984, 4713079-4713170, 4713325-4714095, 4714247-4717711, 4717797- |
| | | | | 4717926, 4718007-4718769, 4718882-4719057, 4719230-4719913, 4720042-4720230, 4720351- |
| | | | | 4720439, 4720555-4721295, 4721402-4721590, 4721681-4721787, 4721903-4722067, 4722119- |
| | | | | 4722377, 4722514-4722722, 4722878-4725086, 4725203-4725933, |
| 28 | MINK1 | NM_015716 | chr17 | 4736936-4781610, 4781678-4784277, 4784336-4784425, 4784553-4787656, 4787769-4788205, 4788298- |
| | | | | 4788776, 4788909-4789204, 4789261-4789413, 4789494-4789744, 4789922-4790403, 4790475- |
| | | | | 4790873, 4791086-4792940, 4793059-4793810, 4794031-4794244, 4794420-4794749, 4795012- |
| | | | | 4795442, 4795530-4795949, 4796064-4796272, 4796363-4796727, 4796893-4797281, 4797389- |
| | | | | 4797467, 4797607-4797818, 4797929-4798368, 4798538-4798643, 4798789-4798991, 4799128- |
| | | | | 4799296, 4799399-4799486, 4799638-4799722, 4799884-4799967, 4800109-4800497, |
| 28 | MINK1 | NM_153827 | chr17 | 4736936-4781610, 4781678-4784277, 4784336-4784425, 4784553-4787656, 4787769-4788205, 4788298- |
| | | | | 4788776, 4788909-4789204, 4789261-4789413, 4789494-4789744, 4789922-4790403, 4790475- |
| | | | | 4790873, 4791086-4792940, 4793059-4793810, 4794031-4794244, 4794420-4794749, 4795012- |
| | | | | 4795442, 4795530-4795695, 4795808-4795949, 4796064-4796272, 4796363-4796727, 4796893- |
| | | | | 4797281, 4797389-4797467, 4797607-4797818, 4797929-4798368, 4798538-4798643, 4798789- |
| | | | | 4798991, 4799128-4799296, 4799399-4799486, 4799638-4799722, 4799884-4799967, 4800109-4800497, |
| 28 | MINK1 | NM_170663 | chr17 | 4736936-4781610, 4781678-4784277, 4784336-4784425, 4784553-4787656, 4787769-4788205, 4788298- |
| | | | | 4788776, 4788909-4789204, 4789261-4789413, 4789494-4789744, 4789922-4790403, 4790475- |
| | | | | 4790873, 4791086-4792940, 4793059-4793810, 4794031-4794244, 4794420-4794749, 4795012- |
| | | | | 4795442, 4795530-4795949, 4796064-4796272, 4796363-4796579, 4796605-4796727, 4796893- |
| | | | | 4797281, 4797389-4797467, 4797607-4797818, 4797929-4798368, 4798538-4798643, 4798789- |
| | | | | 4798991, 4799128-4799296, 4799399-4799486, 4799638-4799722, 4799884-4799967, 4800109-4800497, |
| 28 | MINK1 | NM_001024937 | chr17 | 4736936-4781610, 4781678-4784277, 4784336-4784425, 4784553-4787656, 4787769-4788205, 4788298- |
| | | | | 4788776, 4788909-4789204, 4789261-4789413, 4789494-4789744, 4789922-4790403, 4790475- |
| | | | | 4790873, 4791086-4792940, 4793059-4793810, 4794031-4794244, 4794420-4794809, 4795012- |
| | | | | 4795442, 4795530-4795695, 4795808-4795949, 4796064-4796272, 4796363-4796727, 4796893- |
| | | | | 4797281, 4797389-4797467, 4797607-4797818, 4797929-4798368, 4798538-4798643, 4798789- |
| | | | | 4798991, 4799128-4799296, 4799399-4799486, 4799638-4799722, 4799884-4799967, 4800109-4800497, |
| 28 | ENO3 | NM_053013 | chr17 | 4854478-4855121, 4855210-4856088, 4856186-4856344, 4856405-4856565, 4856637-4857005, 4857141- |
| | | | | 4858368, 4858593-4858700, 4858900-4859235, 4859439-4859866, 4859977-4860124, 4860185-4860271, |
| 28 | ENO3 | NM_001976 | chr17 | 4854520-4855121, 4855210-4856088, 4856186-4856344, 4856405-4856565, 4856637-4857005, 4857141- |
| | | | | 4858368, 4858593-4858700, 4858900-4859235, 4859439-4859866, 4859977-4860124, 4860185-4860271, |
| 28 | ENO3 | NM_001193503 | chr17 | 4854478-4855121, 4855210-4856088, 4856186-4857005, 4857141-4858368, 4858593-4858700, 4858900- |
| | | | | 4859235, 4859439-4859866, 4859977-4860124, 4860185-4860271, |
| 28 | CAMTA2 | NM_001171166 | chr17 | 4872115-4872200, 4872277-4872443, 4872633-4872923, 4872997-4873195, 4873381-4873633, 4873832- |
| | | | | 4875525, 4875832-4876062, 4876287-4876468, 4876549-4876878, 4877065-4877678, 4877796- |
| | | | | 4880351, 4880488-4881797, 4881926-4882977, 4883917-4884518, 4884655-4884971, 4885127- |
| | | | | 4885382, 4885456-4886050, 4886188-4886269, 4886339-4889173, 4889279-4890886, |
| 28 | CAMTA2 | NM_001171168 | chr17 | 4872115-4872200, 4872277-4872443, 4872633-4872793, 4872816-4872923, 4872997-4873195, 4873381- |
| | | | | 4873633, 4873832-4875525, 4875832-4876062, 4876287-4876468, 4876549-4876878, 4877065- |
| | | | | 4877678, 4877796-4880351, 4880488-4881797, 4881926-4882977, 4883917-4884518, 4884655- |
| | | | | 4884971, 4885127-4886050, 4886188-4886269, 4886339-4889173, 4889279-4889485, 4889582-4890247, |
| 28 | CAMTA2 | NM_001171167 | chr17 | 4872115-4872200, 4872275-4872443, 4872633-4872793, 4872816-4872923, 4872997-4873195, 4873381- |
| | | | | 4873633, 4873832-4875525, 4875832-4876062, 4876287-4876468, 4876549-4876878, 4877065- |
| | | | | 4877678, 4877796-4880351, 4880488-4881797, 4881926-4882977, 4883917-4884518, 4884655- |
| | | | | 4884971, 4885127-4885382, 4885456-4886050, 4886188-4886269, 4886339-4889173, 4889279- |
| | | | | 4889485, 4889582-4890247, |
| 28 | CAMTA2 | NM_015099 | chr17 | 4872115-4872200, 4872277-4872443, 4872633-4872793, 4872816-4872923, 4872997-4873195, 4873381- |
| | | | | 4873633, 4873832-4875525, 4875832-4876062, 4876287-4876468, 4876549-4876878, 4877065- |
| | | | | 4877678, 4877796-4880351, 4880488-4881797, 4881926-4882977, 4883917-4884518, 4884655- |
| | | | | 4884971, 4885127-4885382, 4885456-4886050, 4886188-4886269, 4886339-4889173, 4889279- |
| | | | | 4889485, 4889582-4890886, |
| 28 | INCA1 | NM_001167987 | chr17 | 4891850-4892072, 4892240-4892721, 4892920-4893183, 4893225-4893445, 4893561-4897687, 4897771- |
| | | | | 4900296, 4900425-4900789, |
| 28 | INCA1 | NM_001167986 | chr17 | 4891850-4892072, 4892240-4892721, 4892920-4893183, 4893225-4893445, 4893561-4897687, 4897771- |
| | | | | 4899099, 4899226-4900134, 4900425-4900789, |
| 28 | INCA1 | NM_001167985 | chr17 | 4891850-4892072, 4892240-4892721, 4892875-4893183, 4893225-4893445, 4893561-4897687, 4897771- |
| | | | | 4900296, 4900425-4900789, |
| 28 | INCA1 | NM_213726 | chr17 | 4891850-4892072, 4892240-4892721, 4892875-4893183, 4893225-4893445, 4893561-4897687, 4897771- |
| | | | | 4900134, 4900425-4900789, |
| 28 | KIF1C | NM_006612 | chr17 | 4901452-4903143, 4903266-4903513, 4903648-4904065, 4904144-4904515, 4904697-4905352, 4905420- |
| | | | | 4905757, 4905938-4906024, 4906138-4906905, 4906985-4907144, 4907212-4907291, 4907369- |
| | | | | 4907860, 4907941-4908148, 4908296-4910208, 4910380-4910556, 4910638-4910760, 4910838- |
| | | | | 4916946, 4917028-4918036, 4918133-4923289, 4923375-4923785, 4923974-4924099, 4924174- |
| | | | | 4925385, 4926005-4926761, |
| 28 | ZFP3 | NM_153018 | chr17 | 4981871-4994790, |
| 28 | ZNF594 | NM_032530 | chr17 | 5087572-5095041, |
| 28 | LOC100130950 | NR_034082 | chr17 | 5095459-5113043, 5113191-5117343, 5117505-5118440, 5118589-5126610, 5126780-5137117, 5137238- |
| | | | | 5137886, 5138107-5138241, |
| 28 | RABEP1 | NM_001083585 | chr17 | 5185816-5211987, 5212118-5235242, 5235448-5238477, 5238640-5241312, 5241434-5250083, 5250221- |
| | | | | 5253744, 5253925-5257652, 5257786-5264501, 5264971-5266205, 5266312-5268415, 5268534- |
| | | | | 5271663, 5271764-5276607, 5276750-5280409, 5280601-5281470, 5281528-5284682, 5284801- |
| | | | | 5286415, 5286864-5286887, |
| 28 | RABEP1 | NM_004703 | chr17 | 5185816-5211987, 5212118-5235242, 5235448-5238477, 5238640-5241312, 5241434-5250083, 5250221- |
| | | | | 5253744, 5253925-5257652, 5257786-5264501, 5264971-5266205, 5266312-5268415, 5268534- |
| | | | | 5271663, 5271764-5276607, 5276750-5280409, 5280601-5281470, 5281528-5283657, 5283758- |
| | | | | 5284682, 5284801-5286415, 5286864-5286887, |
| 28 | KIAA0753 | NM_014804 | chr17 | 6483185-6493097, 6493324-6493828, 6493951-6498290, 6498374-6499476, 6499664-6502555, 6502641- |
| | | | | 6503698, 6503779-6510191, 6510283-6510499, 6510591-6511666, 6511785-6513312, 6513481- |
| | | | | 6515237, 6515469-6524106, 6524319-6526200, 6526418-6526827, 6526892-6528073, 6528182- |
| | | | | 6531435, 6532062-6538329, 6538492-6543955, |
| 28 | ALOX12 | NM_000697 | chr17 | 6899572-6900143, 6900347-6901826, 6901910-6902032, 6902157-6902270, 6902376-6902623, 6902786- |
| | | | | 6903653, 6903799-6904919, 6905131-6908574, 6908663-6909175, 6909347-6909803, 6909927- |
| | | | | 6913064, 6913167-6913273, 6913446-6913561, |
| 28 | C17orf49 | NM_001142798 | chr17 | 6918198-6918379, 6918476-6919091, 6919195-6919812, 6920005-6920227, 6920334-6920574, |
| 28 | C17orf49 | NM_174893 | chr17 | 6918198-6918379, 6918476-6919091, 6919195-6919812, 6920005-6920227, 6920334-6920578, |
| 28 | C17orf49 | NM_001142799 | chr17 | 6918198-6918379, 6918476-6919812, 6920005-6920227, 6920334-6920578, |
| 28 | CLEC10A | NM_182906 | chr17 | 6978542-6978677, 6978786-6979048, 6979205-6979302, 6979472-6980050, 6980124-6980209, 6980307- |
| | | | | 6981314, 6981433-6982063, 6982205-6983369, |
| 28 | CLEC10A | NM_006344 | chr17 | 6978542-6978677, 6978786-6979048, 6979205-6979293, 6979391-6980050, 6980124-6980209, 6980307- |
| | | | | 6981314, 6981433-6982063, 6982205-6983369, |
| 28 | GPS2 | NM_004489 | chr17 | 7216159-7216346, 7216444-7216529, 7216611-7216697, 7216789-7216885, 7217041-7217223, 7217308- |
| | | | | 7217397, 7217479-7217608, 7217723-7217805, 7217917-7218276, 7218439-7218648, |
| 28 | NEURL4 | NM_032442 | chr17 | 7219647-7220522, 7220690-7220778, 7220893-7221105, 7221252-7221382, 7221492-7221586, 7221676- |
| | | | | 7221812, 7221994-7222367, 7222528-7224076, 7224243-7224429, 7224593-7224678, 7224779- |
| | | | | 7224877, 7225107-7225182, 7225330-7226052, 7226186-7226265, 7226443-7226802, 7226883- |
| | | | | 7226964, 7227043-7227131, 7227332-7227425, 7227590-7227712, 7227800-7228116, 7228300- |
| | | | | 7228526, 7228793-7228958, 7229033-7229572, 7229663-7229754, 7229868-7230028, 7230329- |
| | | | | 7230482, 7230550-7230757, 7231204-7232348, |
| 28 | NEURL4 | NM_001005408 | chr17 | 7219647-7220522, 7220690-7220778, 7220893-7221105, 7221252-7221382, 7221492-7221586, 7221676- |
| | | | | 7221812, 7221994-7222367, 7222528-7224076, 7224243-7224429, 7224593-7224678, 7224779- |
| | | | | 7224877, 7225107-7225182, 7225330-7226052, 7226186-7226271, 7226443-7226802, 7226883- |
| | | | | 7226964, 7227043-7227131, 7227332-7227425, 7227590-7227712, 7227800-7228116, 7228300- |
| | | | | 7228526, 7228793-7228958, 7229033-7229572, 7229663-7229754, 7229868-7230028, 7230329- |
| | | | | 7230482, 7230550-7230757, 7231204-7232348, |
| 28 | NLGN2 | NM_020795 | chr17 | 7312032-7315474, 7315527-7317661, 7317813-7317980, 7318168-7318273, 7318468-7318828, 7319427- |
| | | | | 7320243, |
| 28 | TNFSF12_TNFSF13 | NM_172089 | chr17 | 7452630-7452789, 7452839-7453435, 7453513-7454114, 7454170-7454258, 7454296-7460093, 7460220- |
| | | | | 7462939, 7463020-7463161, 7463211-7463364, 7463485-7463665, 7463806-7464039, |
| 28 | CYB5D1 | NM_144607 | chr17 | 7761613-7761719, 7761798-7761922, 7762143-7762698, |
| 28 | C17orf68 | NM_025099 | chr17 | 8131638-8131819, 8131948-8132043, 8132211-8132458, 8132525-8132618, 8132765-8133207, 8133287- |
| | | | | 8133610, 8133787-8133879, 8133970-8134592, 8134788-8135042, 8135134-8135219, 8135546- |
| | | | | 8135677, 8135794-8136222, 8136351-8137771, 8137974-8138065, 8138245-8138369, 8138604- |
| | | | | 8139147, 8139278-8139374, 8139661-8140691, 8140838-8141347, 8141561-8141708, 8141948- |
| | | | | 8146301, 8146467-8151320, |
| 28 | WDR16 | NM_145054 | chr17 | 9480083-9489088, 9489290-9490013, 9490152-9497508, 9497639-9501549, 9501651-9503382, 9503501- |
| | | | | 9511434, 9511537-9515624, 9515797-9531987, 9532138-9536203, 9536351-9538720, 9538874- |
| | | | | 9541924, 9542029-9545039, 9545153-9546338, |
| 28 | WDR16 | NM_001080556 | chr17 | 9480083-9490017, 9490152-9497508, 9497639-9501549, 9501651-9503382, 9503501-9511434, 9511537- |
| | | | | 9515624, 9515797-9531987, 9532138-9536203, 9536351-9538720, 9538874-9541924, 9542029- |
| | | | | 9545039, 9545153-9546338, |
| 28 | MYH2 | NM_001100112 | chr17 | 10424750-10426405, 10426503-10426623, 10426730-10426811, 10426984-10427074, 10427202- |
| | | | | 10427781, 10427987-10428072, 10428383-10428539, 10428666-10428766, 10428934-10429008, 10429194- |
| | | | | 10429914, 10430113-10430253, 10430374-10431063, 10431192-10432005, 10432397-10432477, 10432570- |
| | | | | 10432651, 10432799-10432879, 10433058-10433147, 10433392-10434948, 10435206-10436600, 10436739- |
| | | | | 10436824, 10436950-10438388, 10438508-10438593, 10438683-10439845, 10439924-10440548, 10440860- |
| | | | | 10440980, 10441153-10442520, 10442672-10442759, 10442880-10443243, 10443384-10443909, 10444015- |
| | | | | 10446190, 10446291-10446413, 10446479-10447026, 10447121-10447217, 10447334-10447415, 10447445- |
| | | | | 10448661, 10448820-10450790, 10450936-10451032, 10451258-10452690, 10452735-10452874, |
| 28 | MYH2 | NM_017534 | chr17 | 10424750-10426405, 10426503-10426623, 10426730-10426811, 10426984-10427074, 10427202- |
| | | | | 10427781, 10427987-10428072, 10428383-10428539, 10428666-10428766, 10428934-10429008, 10429194- |
| | | | | 10429914, 10430113-10430253, 10430374-10431063, 10431192-10432005, 10432397-10432477, 10432570- |
| | | | | 10432651, 10432799-10432879, 10433058-10433147, 10433392-10434948, 10435206-10436600, 10436739- |
| | | | | 10436824, 10436950-10438388, 10438508-10438593, 10438683-10439845, 10439924-10440548, 10440860- |
| | | | | 10440980, 10441153-10442520, 10442672-10442759, 10442880-10443243, 10443384-10443909, 10444015- |
| | | | | 10446190, 10446291-10446413, 10446479-10447026, 10447121-10447217, 10447334-10447415, 10447445- |
| | | | | 10448661, 10448820-10450790, 10450936-10451032, 10451258-10452690, 10452735-10452951, |
| 28 | MYH3 | NM_002470 | chr17 | 10531997-10532912, 10533052-10533157, 10533255-10533407, 10533514-10533603, 10533776- |
| | | | | 10534926, 10535054-10535128, 10535334-10535791, 10536102-10536906, 10537033-10537332, 10537500- |
| | | | | 10538155, 10538341-10538682, 10538881-10539050, 10539171-10541124, 10541253-10541358, 10541750- |
| | | | | 10542188, 10542281-10542359, 10542507-10542613, 10542792-10542875, 10543120-10543311, 10543569- |
| | | | | 10543648, 10543787-10543878, 10544004-10544400, 10544520-10544600, 10544690-10545555, 10545628- |
| | | | | 10545732, 10546041-10546141, 10546314-10547666, 10547818-10547899, 10548020-10549022, 10549163- |
| | | | | 10549244, 10549350-10550497, 10550598-10550678, 10550744-10551872, 10551967-10552892, 10553003- |
| | | | | 10553689, 10553719-10554827, 10554986-10555735, 10555881-10558176, 10558390-10559405, |
| 28 | TEKT3 | NM_031898 | chr17 | 15207470-15211979, 15212136-15215574, 15215799-15217402, 15217548-15222392, 15222465- |
| | | | | 15231307, 15231393-15234322, 15234932-15243343, 15243379-15244833, |
| 28 | PIGL | NM_004278 | chr17 | 16120776-16137283, 16137385-16203200, 16203293-16216859, 16216929-16219993, 16220027- |
| | | | | 16221087, 16221223-16229112, |
| 28 | CENPV | NM_181716 | chr17 | 16246256-16247909, 16248026-16251928, 16252000-16253243, 16253344-16256339, |
| 28 | LRRC48 | NM_031294 | chr17 | 17876289-17877164, 17877277-17880894, 17881073-17887140, 17887259-17891244, 17891413- |
| | | | | 17896077, 17896226-17897611, 17897733-17898311, 17898426-17900772, 17900949-17907675, 17907809- |
| | | | | 17909495, 17909568-17910336, 17910462-17919376, 17919510-17919870, |
| 28 | LRRC48 | NM_001130092 | chr17 | 17876289-17877164, 17877277-17880894, 17881073-17887140, 17887259-17891244, 17891413- |
| | | | | 17896077, 17896226-17897611, 17897733-17898311, 17898426-17900772, 17900949-17907675, 17907809- |
| | | | | 17909495, 17909568-17910336, 17910462-17910710, |
| 28 | LRRC48 | NM_001130091 | chr17 | 17876289-17876977, 17877068-17877164, 17877277-17880894, 17881073-17887140, 17887259- |
| | | | | 17891244, 17891413-17896077, 17896226-17897611, 17897733-17898311, 17898426-17900772, 17900949- |
| | | | | 17907675, 17907809-17909495, 17909568-17910336, 17910462-17910710, |
| 28 | LRRC48 | NM_001130090 | chr17 | 17876289-17876977, 17877068-17877164, 17877277-17880894, 17881073-17887140, 17887259- |
| | | | | 17891244, 17891413-17896077, 17896226-17897611, 17897733-17898311, 17898426-17900772, 17900949- |
| | | | | 17907675, 17907809-17909495, 17909568-17910336, 17910462-17919376, 17919510-17919870, |
| 28 | ALKBH5 | NM_017758 | chr17 | 18088328-18098267, 18098350-18110127, 18110285-18111531, |
| 28 | FLII | NM_002018 | chr17 | 18148587-18148666, 18148734-18148867, 18148975-18149056, 18149165-18149246, 18149377- |
| | | | | 18149474, 18149537-18149620, 18149777-18149906, 18150143-18150225, 18150367-18150481, 18150672- |
| | | | | 18150969, 18151163-18151241, 18151348-18151862, 18152036-18152112, 18152198-18152439, 18152516- |
| | | | | 18152638, 18152723-18154150, 18154332-18154577, 18154792-18154992, 18155131-18155311, 18155461- |
| | | | | 18155784, 18155871-18156613, 18156773-18156856, 18157034-18157391, 18157497-18157833, 18157997- |
| | | | | 18158081, 18158169-18158495, 18158578-18159838, 18159912-18160221, 18160334-18161940, |
| 29 | FLJ31306 | NR_029435 | chr14 | 58734112-58740695, 58740854-58758351, 58758426-58764671, |
| 29 | FLJ31306 | NR_029434 | chr14 | 58734112-58740695, 58740729-58752307, 58752475-58758351, 58758426-58764671, |
| 29 | TOMM20L | NM_207377 | chr14 | 58862812-58863014, 58863060-58869396, 58869480-58874042, 58874187-58875245, |
| 29 | JKAMP | NM_001098625 | chr14 | 59951312-59953447, 59953523-59954390, 59954547-59961734, 59961943-59965443, 59965627- |
| | | | | 59967907, 59967986-59970570, |
| 29 | JKAMP | NM_016475 | chr14 | 59951312-59953429, 59953523-59954390, 59954547-59961734, 59961943-59965443, 59965627- |
| | | | | 59967907, 59967986-59970570, |
| 29 | C14orf38 | NM_001164399 | chr14 | 59972034-59977362, 59977527-59978476, 59978522-59988290, 59988395-59991998, 59992153- |
| | | | | 59993811, 59993893-59998488, 59998629-60004739, 60004873-60005421, 60005559-60007391, 60007465- |
| | | | | 60010060, 60010173-60011879, 60012018-60018071, 60018155-60027835, 60027947-60030453, 60030578- |
| | | | | 60031763, 60031994-60034961, 60035099-60039418, 60039532-60041659, 60041747-60043335, |
| 29 | RTN1 | NM_021136 | chr14 | 60063506-60069781, 60069842-60069928, 60069977-60070568, 60070640-60072084, 60072225- |
| | | | | 60074001, 60074211-60193635, 60194387-60212424, 60213200-60337106, |
| 29 | RTN1 | NM_206852 | chr14 | 60063506-60069781, 60069842-60069928, 60069977-60070568, 60070640-60072084, 60072225- |
| | | | | 60074001, 60074211-60097162, |
| 29 | RTN1 | NM_206857 | chr14 | 60063506-60069781, 60069842-60069928, 60069977-60070568, 60070640-60072084, 60072225- |
| | | | | 60074001, 60074211-60193635, |
| 29 | C14orf135 | NM_022495 | chr14 | 60559138-60581416, 60581598-60581690, 60582180-60582435, 60582538-60582657, 60582779- |
| | | | | 60585045, 60585411-60587912, 60588018-60590934, 60591970-60592353, 60592542-60600686, |
| 30 | SCFD2 | NM_152540 | chr4 | 53740229-53751912, 53752034-53773622, 53773759-53786890, 53787038-54011498, 54011750- |
| | | | | 54139991, 54140169-54179801, 54179931-54218763, 54218934-54231269, |
| 30 | TMEM165 | NM_018475 | chr4 | 56262564-56277779, 56278007-56283237, 56283415-56283968, 56284153-56290703, 56290811-56291541, |
| 30 | SRP72 | NM_006947 | chr4 | 57333911-57335817, 57335940-57337884, 57338010-57340218, 57340364-57340444, 57340558- |
| | | | | 57342835, 57342869-57344543, 57344670-57344756, 57344816-57349303, 57349437-57350900, 57351031- |
| | | | | 57352489, 57352564-57354113, 57354180-57355552, 57355650-57356497, 57356603-57356732, 57356812- |
| | | | | 57357595, 57357735-57361521, 57361561-57366700, 57366862-57367848, |
| 30 | UBA6 | NM_018227 | chr4 | 68484851-68488547, 68488644-68488851, 68488941-68489842, 68489972-68490710, 68490906- |
| | | | | 68492076, 68492196-68494787, 68494861-68496182, 68496254-68497424, 68497490-68497573, 68497627- |
| | | | | 68499061, 68499192-68499958, 68500041-68500145, 68500237-68501169, 68501277-68504659, 68504843- |
| | | | | 68506867, 68506947-68510414, 68510497-68511654, 68511735-68512440, 68512510-68514784, 68514930- |
| | | | | 68527905, 68527974-68528855, 68528934-68529601, 68529666-68530905, 68531011-68534267, 68534393- |
| | | | | 68536186, 68536311-68539413, 68539496-68543327, 68543441-68544155, 68544252-68547279, 68547310- |
| | | | | 68547835, 68547932-68562361, 68562426-68566765, |
| 30 | TMPRSS11E | NM_014058 | chr4 | 69313242-69327537, 69327664-69332347, 69332471-69334595, 69334665-69337176, 69337342- |
| | | | | 69340439, 69340480-69341977, 69342157-69343085, 69343347-69344565, 69344710-69362359, |
| 30 | UGT2B10 | NM_001075 | chr4 | 69870854-69874573, 69874795-69875632, 69875722-69879739, 69879872-69883954, 69884106- |
| | | | | 69885380, 69886058-69886075, |
| 30 | UGT2B10 | NM_001075 | chr4 | 69682456-69683745, 69683896-69687987, 69688121-69692126, 69692216-69693045, 69693267-69696316, |
| 30 | UGT2B10 | NM_001144767 | chr4 | 69682204-69683745, 69683896-69687987, 69688121-69692126, 69692216-69693045, 69693267-69696316, |
| 30 | UGT2B10 | NM_001144767 | chr4 | 69870854-69874573, 69874795-69875632, 69875722-69879739, 69879872-69883954, 69884106- |
| | | | | 69885632, 69886058-69886075, |
| 30 | UGT2B7 | NM_001074 | chr4 | 69962960-69964256, 69964407-69968523, 69968657-69972891, 69972981-69973819, 69974041-69978173, |
| 30 | UGT2A1 | NM_006798 | chr4 | 70455370-70460193, 70460415-70460878, 70460968-70461966, 70462100-70464962, 70465113-70512646, |
| 30 | AMTN | NM_212557 | chr4 | 71384372-71384478, 71384549-71388470, 71388556-71389583, 71389651-71390587, 71390679- |
| | | | | 71394438, 71394476-71394899, 71394928-71396754, 71397018-71398170, |
| 30 | AMBN | NM_016519 | chr4 | 71458091-71459042, 71459113-71462714, 71462767-71464071, 71464121-71465251, 71465364- |
| | | | | 71467133, 71467372-71468339, 71468380-71468513, 71468554-71468687, 71468728-71468975, 71469037- |
| | | | | 71469131, 71469178-71469592, 71469639-71471900, |
| 30 | ANKRD17 | NM_198889 | chr4 | 73942008-73942655, 73942822-73943070, 73943251-73944357, 73944608-73950964, 73951164- |
| | | | | 73956382, 73958018-73959794, 73959946-73962832, 73962990-73963788, 73964238-73968091, 73968265- |
| | | | | 73979507, 73979626-73981536, 73981634-73984403, 73984568-73985877, 73986055-73986596, 73986812- |
| | | | | 73987332, 73987477-73990628, 73990776-73990916, 73991030-74000832, 74000983-74007456, 74007561- |
| | | | | 74007957, 74008103-74008355, 74008485-74010460, 74010550-74012479, 74012591-74012956, 74013150- |
| | | | | 74014528, 74014768-74017145, 74017242-74019595, 74019831-74021346, 74021496-74021734, 74021884- |
| | | | | 74026907, 74027066-74043095, 74043251-74123991, |
| 30 | ANKRD17 | NM_032217 | chr4 | 73942008-73942655, 73942822-73943070, 73943251-73944357, 73944608-73950964, 73951164- |
| | | | | 73956382, 73958018-73959794, 73959946-73962832, 73962990-73963788, 73964238-73968091, 73968265- |
| | | | | 73979507, 73979626-73981536, 73981634-73984403, 73984568-73985877, 73986055-73986596, 73986812- |
| | | | | 73987332, 73987477-73990628, 73990776-73990916, 73991030-74000832, 74000983-74005246, 74006001- |
| | | | | 74007456, 74007561-74007957, 74008103-74008355, 74008485-74010460, 74010550-74012479, 74012591- |
| | | | | 74012956, 74013150-74014528, 74014768-74017145, 74017242-74019595, 74019831-74021346, 74021496- |
| | | | | 74021734, 74021884-74026907, 74027066-74043095, 74043251-74123991, |
| 30 | ALB | NM_000477 | chr4 | 74270124-74270831, 74270891-74272344, 74272479-74274309, 74274523-74275070, 74275205- |
| | | | | 74276027, 74276127-74277711, 74277843-74279135, 74279352-74280750, 74280885-74281971, 74282071- |
| | | | | 74283246, 74283387-74283803, 74284029-74285222, 74285357-74285969, 74286039-74286807, |
| 30 | AFM | NM_001133 | chr4 | 74347581-74349656, 74349707-74349973, 74350108-74351577, 74351791-74352682, 74352817- |
| | | | | 74353439, 74353539-74354345, 74354477-74357587, 74357804-74361015, 74361150-74363367, 74363467- |
| | | | | 74364829, 74364964-74365719, 74365945-74367502, 74367637-74368765, 74368828-74369591, |
| 30 | EPGN | NM_001013442 | chr4 | 75174294-75174808, 75174873-75177900, 75178023-75178733, |
| 30 | RCHY1 | NM_001009922 | chr4 | 76407877-76415789, 76415912-76416932, 76416993-76417097, 76417144-76419309, 76419390- |
| | | | | 76434028, 76434146-76434385, 76434507-76439405, |
| 30 | RCHY1 | NM_015436 | chr4 | 76407877-76415789, 76415912-76416819, 76416848-76416932, 76416993-76417097, 76417144- |
| | | | | 76419309, 76419390-76434028, 76434146-76434385, 76434507-76439405, |
| 30 | RCHY1 | NM_001008925 | chr4 | 76407877-76415789, 76415910-76416819, 76416848-76416932, 76416993-76417097, 76417144- |
| | | | | 76419309, 76419390-76434028, 76434146-76434385, 76434507-76439405, |
| 30 | G3BP2 | NM_203504 | chr4 | 76570887-76571520, 76571641-76572211, 76572342-76573821, 76573926-76580248, 76580431- |
| | | | | 76580950, 76581055-76582106, 76582199-76582739, 76582915-76584025, 76584109-76587113, 76587234- |
| | | | | 76598390, |
| 30 | G3BP2 | NM_203505 | chr4 | 76570887-76571520, 76571641-76572211, 76572342-76573821, 76573926-76579165, 76579266- |
| | | | | 76580248, 76580431-76580950, 76581055-76582106, 76582199-76582739, 76582915-76584025, 76584109- |
| | | | | 76587113, 76587234-76598390, |
| 30 | G3BP2 | NM_012297 | chr4 | 76570887-76571520, 76571641-76572211, 76572342-76573821, 76573926-76579165, 76579266- |
| | | | | 76580248, 76580431-76580950, 76581055-76582106, 76582199-76582739, 76582915-76584025, 76584109- |
| | | | | 76587113, 76587234-76598201, |
| 30 | USO1 | NM_003715 | chr4 | 76650070-76678647, 76678727-76691590, 76691693-76691973, 76692078-76692233, 76692291- |
| | | | | 76695827, 76695950-76703831, 76704012-76708213, 76708356-76711301, 76711392-76711794, 76711951- |
| | | | | 76714841, 76715055-76720773, 76720886-76721502, 76721623-76721769, 76721953-76722230, 76722354- |
| | | | | 76725285, 76725426-76726291, 76726457-76727637, 76727726-76730104, 76730205-76731583, 76731693- |
| | | | | 76733311, 76733529-76734357, |
| 30 | NUP54 | NM_017426 | chr4 | 77036648-77038815, 77038928-77039226, 77039348-77045800, 77045910-77051807, 77051903- |
| | | | | 77052381, 77052438-77053674, 77053873-77055326, 77055516-77057337, 77057566-77065300, 77065446- |
| | | | | 77065541, 77065627-77069459, |
| 30 | SCARB2 | NM_005506 | chr4 | 77082905-77084376, 77084537-77087401, 77087455-77089554, 77089630-77091018, 77091139- |
| | | | | 77095295, 77095467-77096942, 77097064-77097588, 77097682-77100668, 77100859-77102105, 77102255- |
| | | | | 77116858, 77117018-77134578, |
| 30 | CXCL13 | NM_006419 | chr4 | 78432943-78526976, 78527084-78528855, 78528990-78531766, 78531849-78532140, |
| 30 | RASGEF1B | NM_152545 | chr4 | 82348947-82353451, 82353526-82354992, 82355118-82355791, 82355889-82361986, 82362084- |
| | | | | 82363449, 82363531-82366678, 82366783-82366895, 82366993-82368656, 82368733-82369221, 82369439- |
| | | | | 82377803, 82377943-82378651, 82378776-82380484, 82380669-82392922, |
| 30 | SEC31A | NM_014933 | chr4 | 83740407-83742188, 83742262-83745706, 83745828-83748520, 83748786-83750151, 83750212- |
| | | | | 83763291, 83763635-83765537, 83765663-83769955, 83770131-83772582, 83772758-83774721, 83774869- |
| | | | | 83776054, 83776183-83778103, 83778284-83778840, 83778918-83782782, 83782862-83783685, 83783726- |
| | | | | 83784469, 83784546-83785513, 83785752-83787959, 83788114-83788306, 83788470-83791476, 83791578- |
| | | | | 83793095, 83793240-83795762, 83795905-83796878, 83796976-83799881, 83800082-83801950, 83802076- |
| | | | | 83803009, 83803091-83812239, |
| 30 | SEC31A | NM_001077208 | chr4 | 83740407-83742188, 83742262-83745706, 83745828-83748520, 83748786-83750151, 83750212- |
| | | | | 83763336, 83763635-83765537, 83765663-83769955, 83770131-83772582, 83772758-83774721, 83774869- |
| | | | | 83776054, 83776183-83778103, 83778284-83778840, 83778918-83782782, 83782862-83783685, 83783726- |
| | | | | 83784469, 83784546-83785513, 83785752-83787959, 83788114-83788306, 83788470-83791476, 83791578- |
| | | | | 83793095, 83793240-83795762, 83795905-83796878, 83796976-83799881, 83800082-83801950, 83802076- |
| | | | | 83803009, 83803094-83812239, |
| 30 | SEC31A | NM_001077206 | chr4 | 83740407-83742188, 83742262-83745706, 83745828-83748520, 83748786-83750151, 83750212- |
| | | | | 83765537, 83765663-83769955, 83770131-83772582, 83772758-83774721, 83774869-83776054, 83776183- |
| | | | | 83778103, 83778284-83778840, 83778918-83782782, 83782862-83783685, 83783726-83784469, 83784546- |
| | | | | 83785513, 83785752-83787959, 83788114-83788306, 83788470-83791476, 83791578-83793095, 83793240- |
| | | | | 83795762, 83795905-83796878, 83796976-83799881, 83800082-83801950, 83802076-83803009, 83803094- |
| | | | | 83812239, |
| 30 | SEC31A | NM_001077207 | chr4 | 83740407-83742188, 83742262-83745706, 83745828-83748520, 83748786-83750151, 83750212- |
| | | | | 83763291, 83763635-83765537, 83765663-83769955, 83770131-83772582, 83772758-83774721, 83774869- |
| | | | | 83776054, 83776183-83778103, 83778284-83778840, 83778918-83782782, 83782862-83783685, 83783726- |
| | | | | 83784469, 83784546-83785513, 83785752-83787959, 83788114-83788306, 83788470-83791476, 83791578- |
| | | | | 83793095, 83793240-83795762, 83795905-83796878, 83796976-83799881, 83800082-83801950, 83802076- |
| | | | | 83803009, 83803094-83812239, |
| 30 | SEC31A | NM_001191049 | chr4 | 83740407-83742188, 83742262-83745706, 83745828-83748520, 83748786-83750151, 83750212- |
| | | | | 83763336, 83763635-83765537, 83765663-83769955, 83770131-83772582, 83772758-83774721, 83774869- |
| | | | | 83776054, 83776183-83778103, 83778284-83778840, 83778918-83782782, 83782862-83783685, 83783726- |
| | | | | 83784469, 83784546-83785513, 83785752-83787959, 83788114-83788306, 83788470-83791476, 83791578- |
| | | | | 83793095, 83793240-83795762, 83795905-83796878, 83796976-83799881, 83800082-83801950, 83802076- |
| | | | | 83812239, |
| 30 | SEC31A | NM_016211 | chr4 | 83740407-83742188, 83742262-83745706, 83745828-83748520, 83748786-83750151, 83750212- |
| | | | | 83763291, 83763635-83765537, 83765663-83769955, 83770131-83772582, 83772758-83774721, 83774869- |
| | | | | 83776054, 83776183-83778103, 83778284-83778840, 83778918-83784469, 83784546-83785513, 83785752- |
| | | | | 83787959, 83788114-83788306, 83788470-83791476, 83791578-83793095, 83793240-83795762, 83795905- |
| | | | | 83796878, 83796976-83799881, 83800082-83801950, 83802076-83803009, 83803094-83812239, |
| 30 | IBSP | NM_004967 | chr4 | 88720786-88723498, 88723568-88723658, 88723711-88723804, 88723884-88727272, 88727337- |
| | | | | 88731756, 88731917-88732512, |
| 31 | VEGFA | NM_001171628 | chr6 | 43739050-43742076, 43742130-43745204, 43745403-43746195, 43746274-43746624, 43746656-43752276, |
| 31 | VEGFA | NM_001171626 | chr6 | 43739050-43742076, 43742130-43745204, 43745403-43746195, 43746274-43746624, 43746656- |
| | | | | 43749691, 43749825-43752276, |
| 31 | VEGFA | NM_001171629 | chr6 | 43739050-43742076, 43742130-43745204, 43745403-43746195, 43746274-43746624, 43746656- |
| | | | | 43749691, 43749825-43752342, |
| 31 | VEGFA | NM_001033756 | chr6 | 43739050-43742076, 43742130-43745204, 43745403-43746195, 43746274-43746624, 43746656- |
| | | | | 43749691, 43749825-43752342, |
| 31 | VEGFA | NM_001171630 | chr6 | 43739050-43742076, 43742130-43745204, 43745403-43746195, 43746274-43752276, |
| 31 | VEGFA | NM_001171627 | chr6 | 43739050-43742076, 43742130-43745204, 43745403-43746195, 43746274-43746624, 43746656- |
| | | | | 43749691, 43749790-43752276, |
| 31 | VEGFA | NM_001171622 | chr6 | 43739050-43742076, 43742130-43745204, 43745403-43746195, 43746274-43752276, |
| 31 | VEGFA | NM_001025368 | chr6 | 43739050-43742076, 43742130-43745204, 43745403-43746195, 43746274-43746624, 43746656- |
| | | | | 43749691, 43749825-43752276, |
| 31 | VEGFA | NM_001025370 | chr6 | 43739050-43742076, 43742130-43745204, 43745403-43746195, 43746274-43746624, 43746656-43752276, |
| 31 | VEGFA | NM_001025369 | chr6 | 43739050-43742076, 43742130-43745204, 43745403-43746195, 43746274-43746624, 43746656- |
| | | | | 43749691, 43749790-43752276, |
| 31 | VEGFA | NM_001025367 | chr6 | 43739050-43742076, 43742130-43745204, 43745403-43746195, 43746274-43746624, 43746656- |
| | | | | 43748467, 43748523-43749691, 43749825-43752276, |
| 31 | VEGFA | NM_001171624 | chr6 | 43739050-43742076, 43742130-43745204, 43745403-43746195, 43746274-43746624, 43746656- |
| | | | | 43748467, 43748541-43749691, 43749825-43752276, |
| 31 | VEGFA | NM_001171625 | chr6 | 43739050-43742076, 43742130-43745204, 43745403-43746195, 43746274-43746624, 43746656- |
| | | | | 43748467, 43748523-43749691, 43749825-43752276, |
| 31 | VEGFA | NM_001171623 | chr6 | 43739050-43742076, 43742130-43745204, 43745403-43746195, 43746274-43746624, 43746656- |
| | | | | 43748467, 43748592-43749691, 43749825-43752276, |
| 31 | VEGFA | NM_003376 | chr6 | 43739050-43742076, 43742130-43745204, 43745403-43746195, 43746274-43746624, 43746656- |
| | | | | 43748467, 43748541-43749691, 43749825-43752276, |
| 31 | VEGFA | NM_001025366 | chr6 | 43739050-43742076, 43742130-43745204, 43745403-43746195, 43746274-43746624, 43746656- |
| | | | | 43748467, 43748592-43749691, 43749825-43752276, |
| 31 | PLA2G7 | NM_005084 | chr6 | 46672434-46672888, 46673039-46675726, 46675899-46677062, 46677156-46678280, 46678396- |
| | | | | 46679231, 46679357-46680004, 46680075-46682195, 46682291-46684119, 46684266-46684710, 46684834- |
| | | | | 46690518, 46690663-46702915, |
| 31 | PLA2G7 | NM_001168357 | chr6 | 46672434-46672888, 46673039-46675726, 46675899-46677062, 46677156-46678280, 46678396- |
| | | | | 46679231, 46679357-46680004, 46680075-46682195, 46682291-46684119, 46684266-46684710, 46684834- |
| | | | | 46690518, 46690663-46703285, |
| 31 | TNFRSF21 | NM_014452 | chr6 | 47200731-47202404, 47202635-47220990, 47221258-47251672, 47252169-47253678, 47254332-47277150, |
| 31 | DEFB112 | NM_001037498 | chr6 | 50011515-50016248, |
| 31 | IL17F | NM_052872 | chr6 | 52101967-52103526, 52103749-52109193, |
| 31 | GSTA5 | NM_153699 | chr6 | 52696769-52697655, 52697789-52698937, 52699081-52701032, 52701167-52702354, 52702408- |
| | | | | 52705523, 52705642-52710852, |
| 31 | PRIM2 | NM_000947 | chr6 | 57182500-57183233, 57183398-57185253, 57185359-57188997, 57189079-57190721, 57190844- |
| | | | | 57244697, 57244795-57246827, 57246967-57372286, 57372356-57393110, 57393185-57398130, 57398318- |
| | | | | 57467078, 57467207-57472357, 57472442-57498965, 57499036-57512470, 57512788-57512795, |
| 32 | FGF12 | NM_021032 | chr3 | 191861917-191888245, 191888446-192053148, 192053254-192078215, 192078328-192125812, |
| 32 | FGF12 | NM_004113 | chr3 | 191861917-191888245, 191888446-192053148, 192053254-192078215, 192078328- |
| | | | | 192444968, 192445113-192445271, |
| 32 | ACAP2 | NM_012287 | chr3 | 195000158-195006523, 195006587-195009848, 195009961-195012433, 195012545- |
| | | | | 195012992, 195013182-195015446, 195015541-195016435, 195016622-195017917, 195018011- |
| | | | | 195022302, 195022374-195022693, 195022904-195027238, 195027346-195027950, 195028053- |
| | | | | 195029513, 195029567-195041428, 195041543-195047612, 195047689-195053755, 195053853- |
| | | | | 195057659, 195057706-195063198, 195063384-195065986, 195066047-195101736, 195101792- |
| | | | | 195102630, 195102752-195112817, 195112877-195163522, |
| 32 | TFRC | NM_003234 | chr3 | 195779056-195780287, 195780430-195781949, 195782173-195785153, 195785237- |
| | | | | 195785443, 195785504-195787049, 195787119-195789451, 195789517-195789723, 195789811- |
| | | | | 195791178, 195791300-195792312, 195792472-195794387, 195794529-195794901, 195795002- |
| | | | | 195796324, 195796440-195798265, 195798370-195798872, 195799024-195800799, 195800997- |
| | | | | 195802028, 195802232-195803933, 195803994-195808700, |
| 32 | TFRC | NM_001128148 | chr3 | 195779056-195780287, 195780430-195781949, 195782173-195785153, 195785237- |
| | | | | 195785443, 195785504-195787049, 195787119-195789451, 195789517-195789723, 195789811- |
| | | | | 195791178, 195791300-195792312, 195792472-195794387, 195794529-195794901, 195795002- |
| | | | | 195796324, 195796440-195798265, 195798370-195798872, 195799024-195800799, 195800997- |
| | | | | 195802028, 195802232-195803933, 195803994-195808912, |
| 32 | ZDHHC19 | NM_001039617 | chr3 | 195924597-195925145, 195925323-195925658, 195925746-195934267, 195934375- |
| | | | | 195935257, 195935432-195936245, 195936387-195937485, 195937609-195938039, |
| 32 | RNF168 | NM_152617 | chr3 | 196199644-196202100, 196202184-196210639, 196210763-196214268, 196214450- |
| | | | | 196215476, 196215555-196229742, |
| 32 | SENP5 | NM_152699 | chr3 | 196594945-196612020, 196613566-196626535, 196626643-196626793, 196626934- |
| | | | | 196627234, 196627284-196630402, 196630482-196650283, 196650423-196654665, 196654751- |
| | | | | 196656502, 196656555-196657682, |
| 32 | DLG1 | NM_004087 | chr3 | 196771601-196778446, 196778540-196786758, 196786870-196792145, 196792320- |
| | | | | 196792577, 196792681-196793573, 196793626-196796088, 196796132-196803455, 196803557- |
| | | | | 196807920, 196807989-196812449, 196812628-196817781, 196817898-196831772, 196831877- |
| | | | | 196842796, 196842955-196846279, 196846402-196857396, 196857543-196863411, 196863550- |
| | | | | 196865091, 196865263-196867009, 196867136-196869587, 196869640-196876612, 196876668- |
| | | | | 196888509, 196888610-196921294, 196921461-197009548, 197009717-197023215, 197023349- |
| | | | | 197024055, 197024107-197025287, |
| 32 | DLG1 | NM_001098424 | chr3 | 196771601-196778446, 196778540-196786758, 196786870-196792145, 196792320- |
| | | | | 196792577, 196792681-196793573, 196793626-196796088, 196796132-196802706, 196802742- |
| | | | | 196807920, 196807989-196812449, 196812628-196817781, 196817898-196831772, 196831877- |
| | | | | 196842796, 196842955-196846279, 196846402-196857396, 196857543-196863411, 196863550- |
| | | | | 196865091, 196865263-196867009, 196867136-196869587, 196869640-196876612, 196876668- |
| | | | | 196888509, 196888610-196921294, 196921461-197009548, 197009717-197023215, 197023349- |
| | | | | 197024055, 197024107-197025287, |
| 32 | KIAA0226 | NM_014687 | chr3 | 197402068-197402291, 197402387-197403754, 197403910-197404349, 197404413- |
| | | | | 197407998, 197408239-197408704, 197408771-197409339, 197409487-197410176, 197410311- |
| | | | | 197411026, 197411089-197420584, 197420688-197421244, 197421457-197422735, 197422853- |
| | | | | 197423823, 197423921-197427482, 197428018-197428577, 197428736-197430417, 197430526- |
| | | | | 197431411, 197431573-197431961, 197432047-197444846, 197445002-197463524, |
| 32 | KIAA0226 | NM_001145642 | chr3 | 197402068-197402291, 197402387-197403754, 197403910-197404349, 197404413- |
| | | | | 197407998, 197408239-197408704, 197408771-197409339, 197409487-197410176, 197410311- |
| | | | | 197411026, 197411089-197420584, 197420688-197421244, 197421457-197422735, 197422853- |
| | | | | 197423823, 197423921-197426056, 197426103-197427482, 197428018-197428577, 197428736- |
| | | | | 197430417, 197430526-197431411, 197431573-197431961, 197432047-197444846, 197445002- |
| | | | | 197476138, |
| 32 | LRCH3 | NM_032773 | chr3 | 197518412-197541777, 197541924-197544045, 197544174-197547194, 197547302- |
| | | | | 197553747, 197553886-197556433, 197556545-197557639, 197557735-197559066, 197559189- |
| | | | | 197562543, 197562694-197566190, 197566269-197574278, 197574332-197574741, 197574893- |
| | | | | 197579430, 197579492-197581261, 197581317-197585703, 197585777-197592292, 197592343- |
| | | | | 197592981, 197593091-197597029, 197597150-197598194, |
| 32 | IQCG | NM_032263 | chr3 | 197616586-197618315, 197618419-197619497, 197619631-197639544, 197639621- |
| | | | | 197640750, 197640914-197652893, 197652977-197659043, 197659174-197665417, 197665652- |
| | | | | 197670647, 197670923-197672489, 197672558-197686398, 197686591-197686714, |
| 32 | IQCG | NM_001134435 | chr3 | 197616586-197618315, 197618419-197619497, 197619631-197639544, 197639621- |
| | | | | 197640750, 197640914-197652893, 197652977-197659043, 197659174-197665417, 197665652- |
| | | | | 197670647, 197670923-197672489, 197672558-197676708, |
| 33 | ZNF415 | NM_001136038 | chr19 | 53613162-53619564, 53619687-53625655, 53625866-53636000, |
| 33 | ZNF415 | NM_001164309 | chr19 | 53613162-53619564, 53619687-53625913, 53625997-53636000, |
| 33 | ZNF415 | NR_028343 | chr19 | 53613162-53618461, 53618561-53618955, 53619065-53619564, 53619687-53625913, 53625997- |
| | | | | 53626752, 53626909-53636107, |
| 33 | ZNF415 | NM_018355 | chr19 | 53613162-53619564, 53619687-53625913, 53625997-53636107, |
| 33 | ZNF347 | NM_001172674 | chr19 | 53645810-53651932, 53652066-53652492, 53652621-53656984, 53657047-53661931, |
| 33 | ZNF347 | NM_032584 | chr19 | 53645810-53651932, 53652063-53652492, 53652621-53656984, 53657047-53662260, |
| 33 | ZNF347 | NM_001172675 | chr19 | 53645810-53651932, 53652066-53652492, 53652621-53656984, 53657047-53662260, |
| 33 | ZNF665 | NM_024733 | chr19 | 53669601-53678696, 53678825-53686135, 53686197-53696563, |
| 33 | ZNF813 | NM_001004301 | chr19 | 53971044-53987002, 53987092-53989884, 53990013-53993627, |
| 33 | PRPF31 | NM_015629 | chr19 | 54619178-54621649, 54621836-54621951, 54622014-54625237, 54625323-54625874, 54625974- |
| | | | | 54626831, 54626940-54627126, 54627298-54627876, 54628036-54629901, 54629993-54631446, 54631576- |
| | | | | 54631678, 54631753-54632430, 54632561-54632645, 54632746-54634736, |
| 33 | LILRA2 | NM_001130917 | chr19 | 55085380-55085541, 55085579-55085766, 55086050-55086196, 55086501-55086721, 55087020- |
| | | | | 55087272, 55087577-55098475, 55098528-55098666, |
| 33 | LILRA2 | NM_006866 | chr19 | 55085380-55085541, 55085579-55085766, 55086050-55086196, 55086501-55086721, 55087020- |
| | | | | 55087272, 55087577-55098666, |
| 34 | NIPBL | NM_133433 | chr5 | 36877281-36953718, 36953863-36955572, 36955740-36958204, 36958334-36961584, 36961686- |
| | | | | 36962223, 36962377-36970976, 36971139-36972045, 36972144-36975876, 36976505-36984776, 36986404- |
| | | | | 36995722, 36995907-37000473, 37000673-37000917, 37000991-37001089, 37001181-37002762, 37002868- |
| | | | | 37003361, 37003450-37006457, 37006691-37007423, 37007577-37008108, 37008191-37008723, 37008826- |
| | | | | 37010187, 37010328-37014783, 37014868-37016138, 37016273-37017119, 37017265-37019411, 37019503- |
| | | | | 37020559, 37020776-37020875, 37020980-37022151, 37022252-37022344, 37022493-37024685, 37024822- |
| | | | | 37026329, 37026430-37027459, 37027515-37036479, 37036590-37038702, 37038841-37044447, 37044590- |
| | | | | 37044736, 37044832-37045543, 37045700-37046209, 37046302-37048602, 37048778-37049211, 37049404- |
| | | | | 37051879, 37051989-37052466, 37052669-37057286, 37057435-37058991, 37059268-37060944, 37061121- |
| | | | | 37063890, 37064081-37064627, |
| 34 | NIPBL | NM_015384 | chr5 | 36877281-36953718, 36953863-36955572, 36955740-36958204, 36958334-36961584, 36961686- |
| | | | | 36962223, 36962377-36970976, 36971139-36972045, 36972144-36975876, 36976505-36984776, 36986404- |
| | | | | 36995722, 36995907-37000473, 37000673-37000917, 37000991-37001089, 37001181-37002762, 37002868- |
| | | | | 37003361, 37003450-37006457, 37006691-37007423, 37007577-37008108, 37008191-37008723, 37008826- |
| | | | | 37010187, 37010328-37014783, 37014868-37016138, 37016273-37017119, 37017265-37019411, 37019503- |
| | | | | 37020559, 37020776-37020875, 37020980-37022151, 37022252-37022344, 37022493-37024685, 37024822- |
| | | | | 37026329, 37026430-37027459, 37027515-37036479, 37036590-37038702, 37038841-37044447, 37044590- |
| | | | | 37044736, 37044832-37045543, 37045700-37046209, 37046302-37048602, 37048778-37049211, 37049404- |
| | | | | 37051879, 37051989-37052466, 37052669-37057286, 37057435-37058991, 37059268-37060944, 37061121- |
| | | | | 37063890, |
| 34 | NUP155 | NM_004298 | chr5 | 37292141-37292979, 37293088-37294429, 37294568-37298968, 37299081-37299548, 37299671- |
| | | | | 37301537, 37301653-37302879, 37303011-37303360, 37303517-37304839, 37304946-37305157, 37305313- |
| | | | | 37307397, 37307535-37309229, 37309370-37310652, 37310846-37314298, 37314431-37318088, 37318188- |
| | | | | 37324092, 37324210-37326001, 37326070-37327729, 37327879-37328458, 37328523-37329290, 37329381- |
| | | | | 37330138, 37330235-37331785, 37331898-37333563, 37333736-37337918, 37338021-37341190, 37341345- |
| | | | | 37342649, 37342749-37348605, 37348699-37349272, 37349348-37350260, 37350368-37351290, 37351459- |
| | | | | 37352837, 37352932-37358181, 37358254-37363988, 37364087-37364347, 37364487-37370766, |
| 34 | NUP155 | NM_153485 | chr5 | 37292141-37292979, 37293088-37294429, 37294568-37298968, 37299081-37299548, 37299671- |
| | | | | 37301537, 37301653-37302879, 37303011-37303360, 37303517-37304839, 37304946-37305157, 37305313- |
| | | | | 37307397, 37307535-37309229, 37309370-37310652, 37310846-37314298, 37314431-37318088, 37318188- |
| | | | | 37324092, 37324210-37326001, 37326070-37327729, 37327879-37328458, 37328523-37329290, 37329381- |
| | | | | 37330138, 37330235-37331785, 37331898-37333563, 37333736-37337918, 37338021-37341190, 37341345- |
| | | | | 37342649, 37342749-37348605, 37348699-37349272, 37349348-37350260, 37350368-37351290, 37351459- |
| | | | | 37352837, 37352932-37358181, 37358254-37363988, 37364087-37364347, 37364487-37370921, |
| 34 | WDR70 | NM_018034 | chr5 | 37379495-37379589, 37379657-37381702, 37381788-37392100, 37392223-37396475, 37396673- |
| | | | | 37438022, 37438084-37443339, 37443475-37479934, 37480090-37516614, 37516693-37605164, 37605341- |
| | | | | 37697755, 37697857-37701158, 37701245-37703049, 37703190-37721215, 37721318-37722955, 37723037- |
| | | | | 37725034, 37725153-37726983, 37727148-37752586, |
| 34 | RPL37 | NM_000997 | chr5 | 40832676-40834281, 40834368-40834571, 40834709-40835283, |
| 34 | HMGCS1 | NM_002130 | chr5 | 43291323-43292574, 43292740-43292948, 43293076-43294156, 43294265-43294791, 43294964- |
| | | | | 43295852, 43296020-43297102, 43297269-43298109, 43298237-43298618, 43299078-43313456, |
| 34 | HMGCS1 | NM_001098272 | chr5 | 43291323-43292574, 43292740-43292948, 43293076-43294156, 43294265-43294791, 43294964- |
| | | | | 43295852, 43296020-43297102, 43297269-43298109, 43298237-43298618, 43299078-43307866, 43307927- |
| | | | | 43313456, |
| 34 | PAIP1 | NM_182789 | chr5 | 43527572-43529886, 43529982-43533838, 43533895-43534953, 43535073-43535634, 43535743- |
| | | | | 43536919, 43537047-43539024, 43539138-43543104, 43543219-43547828, 43548016-43555930, 43556102- |
| | | | | 43556919, |
| 34 | PAIP1 | NM_183323 | chr5 | 43527572-43529886, 43529982-43533838, 43533895-43534953, 43535073-43535634, 43535743- |
| | | | | 43536919, 43537047-43539024, 43539138-43543104, 43543219-43547828, 43548016-43555930, 43556102- |
| | | | | 43557440, |
| 34 | PAIP1 | NM_006451 | chr5 | 43527572-43529886, 43529982-43533838, 43533895-43534953, 43535073-43535634, 43535743- |
| | | | | 43536919, 43537047-43539024, 43539138-43543104, 43543219-43547828, 43548016-43555930, 43556102- |
| | | | | 43556682, |
| 35 | DPM3 | NM_153741 | chr1 | 155112722-155112965, |
| 35 | KRTCAP2 | NM_173852 | chr1 | 155142037-155142265, 155142334-155145039, 155145105-155145212, 155145369-155145695, |
| 35 | MSTO1 | NM_018116 | chr1 | 155580124-155580214, 155580352-155580828, 155580900-155581005, 155581083- |
| | | | | 155581338, 155581395-155581478, 155581619-155581772, 155581892-155581971, 155582108- |
| | | | | 155582207, 155582362-155582633, 155582767-155582838, 155583025-155583251, 155583358- |
| | | | | 155583446, 155583558-155583848, |
| 35 | IQGAP3 | NM_178229 | chr1 | 156496392-156497742, 156497853-156498299, 156498403-156498706, 156498875- |
| | | | | 156499895, 156500110-156500950, 156501040-156502770, 156502899-156503563, 156503668- |
| | | | | 156503799, 156503934-156504291, 156504526-156504953, 156505040-156506971, 156507114- |
| | | | | 156508599, 156508826-156509164, 156509330-156509628, 156509783-156510498, 156510709- |
| | | | | 156513771, 156513843-156513942, 156514016-156514179, 156514265-156517863, 156518036- |
| | | | | 156518138, 156518280-156518371, 156518541-156520051, 156520144-156521495, 156521661- |
| | | | | 156521764, 156521888-156524025, 156524185-156526323, 156526486-156530724, 156530814- |
| | | | | 156531628, 156531794-156532377, 156532458-156532924, 156533105-156533343, 156533459- |
| | | | | 156533547, 156533617-156534405, 156534484-156535817, 156535897-156536180, 156536339- |
| | | | | 156539166, 156539256-156542283, |
| 35 | ITLN2 | NM_080878 | chr1 | 160915083-160917717, 160917823-160919834, 160919957-160920341, 160920502- |
| | | | | 160920831, 160921081-160922408, 160922524-160924175, 160924241-160924515, |
| 35 | PVRL4 | NM_030916 | chr1 | 161042676-161043013, 161043090-161043508, 161043586-161044005, 161044164- |
| | | | | 161044399, 161044550-161046143, 161046266-161047241, 161047534-161049378, 161049740- |
| | | | | 161059006, |
| 35 | NUF2 | NM_145697 | chr1 | 163292003-163295820, 163295965-163297276, 163297353-163298016, 163298095- |
| | | | | 163298634, 163298698-163306539, 163306639-163307808, 163307884-163309169, 163309268- |
| | | | | 163310152, 163310217-163313521, 163313661-163315466, 163315609-163317551, 163317729- |
| | | | | 163318733, 163318871-163325123, |
| 35 | NUF2 | NM_031423 | chr1 | 163291857-163295820, 163295965-163297276, 163297353-163298016, 163298095- |
| | | | | 163298634, 163298698-163306539, 163306639-163307808, 163307884-163309169, 163309268- |
| | | | | 163310152, 163310217-163313521, 163313661-163315466, 163315609-163317551, 163317729- |
| | | | | 163318733, 163318871-163325123, |
| 35 | MPZL1 | NM_003953 | chr1 | 167691480-167734818, 167734987-167741510, 167741726-167742471, 167742606- |
| | | | | 167745299, 167745404-167757055, |
| 35 | MPZL1 | NM_024569 | chr1 | 167691480-167734818, 167734987-167741510, 167741726-167742471, 167742606-167757055, |
| 35 | MPZL1 | NM_001146191 | chr1 | 167691480-167734818, 167734987-167757055, |
| 35 | TBX19 | NM_005149 | chr1 | 168250532-168260396, 168260663-168262380, 168262517-168266960, 168267024- |
| | | | | 168269658, 168269722-168274244, 168274435-168277978, 168278116-168281944, |
| 35 | C1orf9 | NM_016227 | chr1 | 172502638-172520650, 172520767-172525007, 172525164-172526418, 172526558- |
| | | | | 172526782, 172526935-172538157, 172538283-172539185, 172539312-172539801, 172539871- |
| | | | | 172543029, 172543139-172544656, 172544764-172546902, 172546960-172547436, 172547530- |
| | | | | 172548340, 172548408-172554156, 172554227-172554996, 172555089-172557898, 172559067- |
| | | | | 172560122, 172560212-172569202, 172569309-172571202, 172571359-172571834, 172571928- |
| | | | | 172577885, 172577943-172578954, |
| 35 | C1orf9 | NM_014283 | chr1 | 172502638-172520650, 172520767-172522398, 172522511-172525007, 172525164- |
| | | | | 172526418, 172526558-172526782, 172526935-172538157, 172538283-172539185, 172539312- |
| | | | | 172539801, 172539871-172543029, 172543139-172544656, 172544764-172546677, 172546700- |
| | | | | 172546902, 172546960-172547436, 172547530-172548340, 172548408-172554156, 172554227- |
| | | | | 172554996, 172555089-172557898, 172559067-172560122, 172560212-172569202, 172569309- |
| | | | | 172571202, 172571359-172571834, 172571928-172577885, 172577943-172578954, |
| 35 | PRDX6 | NM_004905 | chr1 | 173446632-173450463, 173450622-173454498, 173454647-173455392, 173455541-173456872, |
| 36 | MCF2 | NM_001171879 | chrX | 138664663-138667223, 138667338-138668535, 138668647-138669873, 138669948- |
| | | | | 138670517, 138670598-138671992, 138672087-138678706, 138678930-138679617, 138679745- |
| | | | | 138680563, 138680658-138684563, 138684658-138686838, 138686910-138687026, 138687144- |
| | | | | 138687870, 138687939-138689848, 138689939-138692458, 138692498-138695373, 138695423- |
| | | | | 138697038, 138697212-138698439, 138698633-138699670, 138699864-138701744, 138701866- |
| | | | | 138708350, 138708467-138708780, 138708916-138711852, 138712004-138713552, 138713671- |
| | | | | 138714492, 138714614-138724625, |
| 36 | MCF2 | NM_001171877 | chrX | 138664663-138669873, 138669948-138670517, 138670598-138671992, 138672087- |
| | | | | 138678706, 138678930-138679617, 138679745-138680563, 138680658-138684563, 138684658- |
| | | | | 138686838, 138686910-138687026, 138687144-138687870, 138687939-138689848, 138689939- |
| | | | | 138692458, 138692498-138697038, 138697212-138698439, 138698633-138699670, 138699864- |
| | | | | 138701744, 138701866-138708350, 138708467-138708780, 138708916-138711852, 138712004- |
| | | | | 138714492, 138714614-138724625, |
| 36 | MCF2 | NM_001171878 | chrX | 138664663-138669873, 138669948-138670517, 138670598-138671992, 138672087- |
| | | | | 138678706, 138678930-138679617, 138679745-138680563, 138680658-138684563, 138684658- |
| | | | | 138686838, 138686910-138687026, 138687144-138687870, 138687939-138689848, 138689939- |
| | | | | 138692458, 138692498-138697038, 138697212-138698439, 138698633-138699670, 138699864- |
| | | | | 138701744, 138701866-138708350, 138708467-138708780, 138708916-138711852, 138712004- |
| | | | | 138713552, 138713671-138714492, 138714614-138724625, |
| 36 | MCF2 | NM_005369 | chrX | 138664663-138667223, 138667338-138668535, 138668647-138669873, 138669948- |
| | | | | 138670517, 138670598-138671992, 138672087-138678706, 138678930-138679617, 138679745- |
| | | | | 138680563, 138680658-138684563, 138684658-138686838, 138686910-138687026, 138687144- |
| | | | | 138687870, 138687939-138689848, 138689939-138692458, 138692498-138697038, 138697212- |
| | | | | 138698439, 138698633-138699670, 138699864-138701744, 138701866-138708350, 138708467- |
| | | | | 138708780, 138708916-138711852, 138712004-138713552, 138713671-138714492, 138714614- |
| | | | | 138724625, |
| 36 | MCF2 | NM_001099855 | chrX | 138664663-138667223, 138667338-138668535, 138668647-138669873, 138669948- |
| | | | | 138670517, 138670598-138671992, 138672087-138678706, 138678930-138679617, 138679745- |
| | | | | 138680563, 138680658-138684563, 138684658-138686838, 138686910-138687026, 138687144- |
| | | | | 138687870, 138687939-138689848, 138689939-138692458, 138692498-138697038, 138697212- |
| | | | | 138698439, 138698633-138699670, 138699864-138701744, 138701866-138708350, 138708467- |
| | | | | 138708780, 138708916-138711852, 138712004-138713552, 138713671-138714492, 138714614- |
| | | | | 138727725, 138727818-138728946, 138729063-138733877, 138733948-138790263, |
| 36 | MCF2 | NM_001171876 | chrX | 138664663-138667223, 138667338-138668535, 138668647-138669873, 138669948- |
| | | | | 138670517, 138670598-138671992, 138672087-138678706, 138678930-138679617, 138679745- |
| | | | | 138680563, 138680658-138684563, 138684658-138686838, 138686910-138687026, 138687144- |
| | | | | 138687870, 138687939-138689848, 138689939-138692458, 138692498-138695373, 138695423- |
| | | | | 138697038, 138697212-138698439, 138698633-138699670, 138699864-138701744, 138701866- |
| | | | | 138708350, 138708467-138708780, 138708916-138711852, 138712004-138713552, 138713671- |
| | | | | 138714492, 138714614-138727725, 138727818-138728946, 138729063-138733877, 138733948- |
| | | | | 138790263, |
| 36 | RP1_177G6_2 | NR_028345 | chrX | 139791971-139793784, |
| 36 | RP1_177G6_2 | NR_028344 | chrX | 139791971-139793784, 139793822-139795680, |
| 36 | SPANXN2 | NM_001009615 | chrX | 142795600-142803683, |
| 36 | SLITRK2 | NM_001144009 | chrX | 144903403-144903899, |
| 36 | SLITRK2 | NM_001144005 | chrX | 144899545-144901990, 144902055-144903347, 144903403-144903521, 144903682-144903899, |
| 36 | SLITRK2 | NM_001144008 | chrX | 144903403-144903521, 144903682-144903899, |
| 36 | SLITRK2 | NM_001144010 | chrX | 144903403-144903521, 144903672-144903911, |
| 36 | SLITRK2 | NM_001144006 | chrX | 144903403-144903521, 144903682-144903911, |
| 36 | SLITRK2 | NM_001144003 | chrX | 144899545-144901990, 144902055-144903347, 144903403-144903521, 144903672-144903899, |
| 36 | SLITRK2 | NM_001144004 | chrX | 144899545-144901990, 144902055-144903347, 144903403-144903521, 144903672-144903911, |
| 36 | SLITRK2 | NM_032539 | chrX | 144899545-144901990, 144902055-144902864, 144903403-144903521, 144903672-144903899, |
| 36 | MAGEA5 | NM_021049 | chrX | 151284078-151284151, 151284240-151286390, |
| 36 | MAGEA10 | NM_021048 | chrX | 151304158-151304231, 151304307-151305341, 151305442-151306945, |
| 36 | MAGEA10 | NM_001011543 | chrX | 151304158-151304231, 151304307-151304460451304553-151305341, 151305442-151306945, |
| 36 | MAGEA12 | NM_005367 | chrX | 151900876-151903102, |
| 36 | MAGEA12 | NM_001166387 | chrX | 151900876-151901707, 151901815-151903102, |
| 36 | MAGEA12 | NM_001166386 | chrX | 151900876-151901707, 151901815-151902927, |
| 37 | RNF125 | NM_017831 | chr15 | 29598991-29617077, 29617233-29622140, 29622237-29625603, 29625696-29645863, 29645973-29648259, |
| 37 | ZSCAN30 | NM_001166012 | chr15 | 32834346-32843475, 32843622-32843907, 32844420-32847438, 32847644-32870046, |
| 37 | ZSCAN30 | NM_001112734 | chr15 | 32834346-32843475, 32843622-32843907, 32844420-32870046, |
| 37 | ZNF396 | NM_145756 | chr15 | 32948410-32949278, 32949625-32953418, 32953565-32953838, 32954329-32957240, |
| 37 | SETBP1 | NM_015559 | chr15 | 42260987-42281138, 42281798-42449193, 42449249-42529844, 42533306-42618448, 42618621-42643042, |
| 37 | SETBP1 | NM_001130110 | chr15 | 42260361-42281138, 42281798-42449193, 42449249-42456528, |
| 37 | SLC14A2 | NM_007163 | chr15 | 43195548-43204594, 43204780-43205646, 43205829-43206921, 43207113-43212313, 43212444- |
| | | | | 43216953, 43217148-43219709, 43219859-43221172, 43221309-43223095, 43223147-43223949, 43224126- |
| | | | | 43243748, 43243873-43246100, 43246185-43246898, 43247060-43247796, 43247988-43248312, 43248443- |
| | | | | 43249269, 43249464-43252863, 43253013-43253646, 43253783-43258938, 43258990-43262282, |
| 37 | HAUS1 | NR_026978 | chr15 | 43684408-43685158, 43685375-43698145, 43698283-43699890, 43700027-43702431, 43702557- |
| | | | | 43703263, 43703331-43704786, 43704860-43705708, 43705758-43708039, |
| 37 | HAUS1 | NM_138443 | chr15 | 43684408-43685158, 43685335-43698145, 43698283-43699890, 43700027-43702431, 43702557- |
| | | | | 43703263, 43703331-43704786, 43704860-43705708, 43705758-43708039, |
| 38 | CNTNAP1 | NM_003632 | chr17 | 40834915-40835837, 40835941-40836052, 40836248-40837007, 40837157-40837233, 40837439- |
| | | | | 40837973, 40838160-40838919, 40839065-40839736, 40840000-40840478, 40840630-40840892, 40841066- |
| | | | | 40841537, 40841646-40842104, 40842226-40842755, 40842961-40843153, 40843312-40843400, 40843530- |
| | | | | 40843822, 40844010-40844515, 40844739-40845313, 40845555-40847537, 40847893-40848041, 40848171- |
| | | | | 40849290, 40849386-40849570, 40849818-40849919, 40849969-40850634, |
| 38 | LOC100190938 | NR_024462 | chr17 | 40906249-40906536, 40907232-40911755, 40911899-40912442, 40912532-40912651, 40912730-40912896, |
| 38 | LOC100190938 | NR_024461 | chr17 | 40908398-40911755, 40911899-40912442, 40912730-40912896, |
| 38 | RAMP2 | NM_005854 | chr17 | 40913377-40913850, 40913918-40914371, 40914482-40914613, |
| 38 | VAT1 | NM_006373 | chr17 | 41168162-41168322, 41168566-41169856, 41169948-41170051, 41170224-41170607, 41170817-41173951, |
| 38 | TMEM106A | NM_145041 | chr17 | 41363942-41364244, 41364414-41365038, 41365272-41365845, 41365911-41367805, 41367961- |
| | | | | 41368466, 41368609-41368714, 41368760-41369276, 41369332-41369698, |
| 38 | LOC100130581 | NR_027413 | chr17 | 41455182-41456489, 41456631-41458639, 41459029-41465655, |
| 38 | LOC100130581 | NR_027412 | chr17 | 41448408-41448613, 41448795-41455037, 41455182-41456489, 41456631-41458639, 41459029-41465655, |
| 39 | MYOG | NM_002479 | chr1 | 203053160-203053773, 203053857-203054617, |
| 39 | CHI3L1 | NM_001276 | chr1 | 203148714-203148887, 203149006-203149596, 203149781-203150288, 203150414- |
| | | | | 203151857, 203151981-203152767, 203152920-203153702, 203153761-203154310, 203154514- |
| | | | | 203155442, 203155474-203155725, |
| 39 | HSD11B1 | NM_005525 | chr1 | 209878376-209879154, 209879287-209880052, 209880166-209880286, 209880474- |
| | | | | 209905779, 209905925-209907647, |
| 39 | HSD11B1 | NM_181755 | chr1 | 209859631-209878260, 209878376-209879154, 209879287-209880052, 209880166- |
| | | | | 209880286, 209880474-209905779, 209905925-209907647, |
| 39 | NEK2 | NM_002497 | chr1 | 211836995-211840446, 211840574-211842453, 211842675-211843621, 211843750- |
| | | | | 211844542, 211844627-211846823, 211847066-211847636, 211847856-211848724, |
| 39 | CENPF | NM_016343 | chr1 | 214776665-214787055, 214787260-214788173, 214788372-214791914, 214792038- |
| | | | | 214792470, 214792564-214793996, 214794290-214795420, 214795625-214802387, 214802515- |
| | | | | 214803875, 214804006-214805821, 214805946-214811207, 214811345-214813262, 214816668- |
| | | | | 214817898, 214820744-214822016, 214822171-214825051, 214825230-214826169, 214826333- |
| | | | | 214828582, 214828747-214830274, 214830753-214832191, 214832372-214836932, |
| 39 | EPRS | NM_004446 | chr1 | 220142299-220142398, 220142465-220145409, 220145490-220146578, 220146741- |
| | | | | 220151886, 220152062-220152758, 220152958-220153425, 220153583-220154096, 220154200- |
| | | | | 220154718, 220154800-220156112, 220156187-220156529, 220156741-220157546, 220157600- |
| | | | | 220160482, 220160747-220161930, 220162166-220170323, 220170685-220174478, 220174598- |
| | | | | 220178588, 220178703-220179446, 220179656-220180542, 220180681-220184286, 220184399- |
| | | | | 220191789, 220191851-220192335, 220192422-220193328, 220193564-220195687, 220195861- |
| | | | | 220197604, 220197799-220198472, 220198601-220203726, 220203823-220205727, 220205869- |
| | | | | 220206842, 220207001-220208254, 220208356-220213525, 220213612-220219683, |
| 39 | IARS2 | NM_018060 | chr1 | 220267826-220269444, 220269569-220273830, 220273992-220275469, 220275620- |
| | | | | 220275703, 220275755-220275835, 220275947-220276027, 220276120-220276787, 220276905- |
| | | | | 220279231, 220279403-220280401, 220280494-220284126, 220284280-220287654, 220287817- |
| | | | | 220298577, 220298682-220300090, 220300186-220307742, 220307853-220310149, 220310254- |
| | | | | 220311258, 220311386-220312348, 220312482-220313523, 220313632-220315143, 220315291- |
| | | | | 220316284, 220316477-220318849, 220318996-220320833, |
| 39 | RAB3GAP2 | NM_012414 | chr1 | 220324749-220324946, 220325107-220326525, 220326749-220327308, 220327400- |
| | | | | 220330610, 220330831-220331142, 220331219-220332726, 220332764-220335538, 220335611- |
| | | | | 220338073, 220338142-220340633, 220340742-220340842, 220341018-220344232, 220344463- |
| | | | | 220345229, 220345392-220345977, 220346085-220355597, 220355693-220356054, 220356274- |
| | | | | 220357376, 220357506-220358991, 220359084-220363043, 220363110-220363404, 220363489- |
| | | | | 220363717, 220363863-220364408, 220364627-220366580, 220366722-220368418, 220368510- |
| | | | | 220368638, 220368720-220369590, 220369741-220375616, 220375717-220379247, 220379349- |
| | | | | 220383728, 220383832-220384141, 220384219-220384295, 220384345-220386227, 220386311- |
| | | | | 220387196, 220387322-220406139, 220406206-220445563, |
| 39 | AIDA | NM_022831 | chr1 | 222843332-222843473, 222843593-222846653, 222846778-222849436, 222849561- |
| | | | | 222860270, 222860379-222860935, 222861001-222867129, 222867186-222867550, 222867606- |
| | | | | 222876488, 222876560-222885548, |
| 39 | SRP9 | NM_001130440 | chr1 | 225965715-225971000, 225971071-225974562, 225974688-225976940, |
| 39 | SRP9 | NM_003133 | chr1 | 225965715-225971000, 225971071-225976940, |
| 39 | ACBD3 | NM_022735 | chr1 | 226334523-226340034, 226340321-226342346, 226342535-226346883, 226347060- |
| | | | | 226349230, 226349391-226352488, 226352631-226353558, 226353702-226374089, |
| 39 | LIN9 | NM_173083 | chr1 | 226420308-226420795, 226420895-226421043, 226421225-226426670, 226426798- |
| | | | | 226438538, 226438621-226453232, 226453336-226453912, 226454034-226455656, 226455792- |
| | | | | 226465474, 226465634-226474032, 226474160-226475363, 226475499-226483541, 226483648- |
| | | | | 226485418, 226485515-226488872, 226488907-226496808, |
| 39 | C1orf198 | NM_032800 | chr1 | 230975655-230979098, 230979643-230991412, 230991465-231003924, |
| 39 | C1orf198 | NM_001136495 | chr1 | 230975655-230979098, 230979643-230991412, 230991465-230991622, |
| 39 | C1orf198 | NM_001136494 | chr1 | 230975655-230979098, 230979643-230991412, 230991465-231003924, 231004149- |
| | | | | 231004556, 231004662-231005226, |
| 39 | TTC13 | NM_001122835 | chr1 | 231042766-231044456, 231044538-231044686, 231044767-231047214, 231047296- |
| | | | | 231048367, 231048505-231051937, 231052046-231056254, 231056341-231057165, 231057251- |
| | | | | 231059585, 231059680-231060585, 231060720-231061261, 231061394-231064681, 231064840- |
| | | | | 231067042, 231067219-231067480, 231067624-231069523, 231069608-231075130, 231075243- |
| | | | | 231076184, 231076303-231090077, 231090150-231093968, 231094046-231096953, 231097050- |
| | | | | 231114304, |
| 39 | TTC13 | NM_024525 | chr1 | 231042766-231044456, 231044538-231044686, 231044767-231047214, 231047296- |
| | | | | 231048367, 231048505-231051937, 231052049-231056254, 231056341-231057165, 231057251- |
| | | | | 231059585, 231059680-231060585, 231060720-231061261, 231061394-231064681, 231064840- |
| | | | | 231067042, 231067219-231067480, 231067624-231069523, 231069608-231075130, 231075243- |
| | | | | 231076184, 231076303-231079550, 231079645-231081133, 231081201-231090077, 231090150- |
| | | | | 231093968, 231094046-231096953, 231097050-231114304, |
| 39 | C1orf124 | NM_032018 | chr1 | 231474351-231475513, 231475615-231483541, 231483672-231487048, 231487318-231488354, |
| 39 | C1orf124 | NM_001010984 | chr1 | 231474351-231475513, 231475615-231483541, 231483672-231487048, |
| 39 | EGLN1 | NM_022051 | chr1 | 231502222-231503313, 231503383-231506306, 231506445-231509724, 231509846-231556742, |
| 39 | SIPA1L2 | NM_020808 | chr1 | 232535020-232538136, 232538220-232539192, 232539318-232539869, 232539925- |
| | | | | 232551238, 232551372-232561333, 232561555-232564155, 232564311-232567991, 232568218- |
| | | | | 232574852, 232575243-232577035, 232577141-232577512, 232577615-232579346, 232579432- |
| | | | | 232581273, 232581533-232596631, 232596908-232600584, 232601163-232607115, 232607275- |
| | | | | 232615371, 232615477-232619536, 232619713-232626618, 232626809-232629271, 232629407- |
| | | | | 232649601, |
| 39 | IRF2BP2 | NM_001077397 | chr1 | 234743599-234744239, |
| 39 | IRF2BP2 | NM_182972 | chr1 | 234743599-234744191, |
| 39 | RBM34 | NM_001161533 | chr1 | 235316081-235318194, 235318428-235323824, 235323963-235324206, 235324383-235324487, |
| 39 | RBM34 | NM_015014 | chr1 | 235295313-235298965, 235299086-235299347, 235299389-235301190, 235301256- |
| | | | | 235301404, 235301490-235311717, 235311763-235316019, 235316081-235318194, 235318428- |
| | | | | 235323824, 235323963-235324206, 235324383-235324487, |
| 39 | RBM34 | NR_027762 | chr1 | 235295313-235298965, 235299086-235299347, 235299389-235301190, 235301256- |
| | | | | 235301404, 235301490-235311717, 235311763-235316019, 235316081-235318194, 235318428- |
| | | | | 235324206, 235324383-235324487, |
| 39 | B3GALNT2 | NM_152490 | chr1 | 235613656-235616400, 235616459-235617466, 235617628-235618869, 235618997- |
| | | | | 235621909, 235622095-235628951, 235629032-235634162, 235634275-235643368, 235643466- |
| | | | | 235647636, 235647832-235652471, 235652574-235657989, 235658139-235667439, |
| 39 | CHRM3 | NM_000740 | chr1 | 239792840-239795522, 239795587-239841484, 239841589-239990550, 239990679-240070731, |
| 39 | KMO | NM_003679 | chr1 | 241695799-241712127, 241712199-241712975, 241713075-241714253, 241714345- |
| | | | | 241718910, 241718961-241723963, 241724053-241725465, 241725633-241728285, 241728359- |
| | | | | 241729789, 241729913-241731798, 241731948-241749979, 241750039-241752048, 241752133- |
| | | | | 241753312, 241753416-241753504, 241753566-241755253, |
| 39 | OPN3 | NM_014322 | chr1 | 241757994-241761046, 241761300-241767560, 241767882-241803182, |
| 39 | EXO1 | NM_003686 | chr1 | 242012330-242013709, 242013889-242015592, 242015714-242016658, 242016784- |
| | | | | 242020645, 242020785-242021806, 242022021-242023817, 242024007-242024706, 242024805- |
| | | | | 242030130, 242030358-242035332, 242035581-242042049, 242042646-242045216, 242045320- |
| | | | | 242048614, 242048810-242052767, |
| 39 | EXO1 | NM_006027 | chr1 | 242012330-242013709, 242013889-242015592, 242015714-242016658, 242016784- |
| | | | | 242020645, 242020785-242021806, 242022021-242023817, 242024007-242024706, 242024805- |
| | | | | 242030130, 242030358-242035332, 242035581-242042049, 242042646-242045216, 242045320- |
| | | | | 242048614, 242048810-242052765, |
| 39 | EXO1 | NM_130398 | chr1 | 242011656-242012031, 242012330-242012412, 242012519-242013709, 242013889- |
| | | | | 242015592, 242015714-242016658, 242016784-242020645, 242020785-242021806, 242022021- |
| | | | | 242023817, 242024007-242024706, 242024805-242030130, 242030358-242035332, 242035581- |
| | | | | 242042049, 242042646-242045216, 242045320-242048614, 242048810-242052765, |
| 39 | PPPDE1 | NM_016076 | chr1 | 244816658-244849897, 244849972-244852549, 244852645-244855179, 244855323-244868856, |
| 39 | AHCTF1 | NM_015446 | chr1 | 247004301-247005994, 247006079-247007095, 247007231-247012915, 247014745- |
| | | | | 247016391, 247016602-247019030, 247019130-247020991, 247021117-247024199, 247024598- |
| | | | | 247025259, 247025456-247027224, 247027419-247030544, 247030654-247030961, 247031114- |
| | | | | 247039337, 247039490-247040250, 247040384-247040458, 247040605-247048793, 247048983- |
| | | | | 247050511, 247050660-247051477, 247051536-247051694, 247051820-247053266, 247053362- |
| | | | | 247054256, 247054363-247055079, 247055222-247057959, 247058051-247059135, 247059228- |
| | | | | 247061508, 247061639-247062751, 247062815-247063365, 247063549-247063637, 247063772- |
| | | | | 247065825, 247065978-247067249, 247067336-247068841, 247068960-247070851, 247071061- |
| | | | | 247076532, 247076715-247079442, 247079698-247081550, 247081680-247094608, |
| 39 | ZNF695 | NM_020394 | chr1 | 247151558-247162648, 247162743-247163212, 247163377-247171206, |
| 40 | FANCI | NM_001113378 | chr15 | 89787265-89790858, 89790963-89801933, 89802008-89803942, 89804075-89804814, 89804973- |
| | | | | 89805050, 89805110-89806648, 89806692-89807132, 89807258-89807751, 89807839-89811628, 89811757- |
| | | | | 89816606, 89816701-89817397, 89817536-89819940, 89820123-89821916, 89822006-89824399, 89824532- |
| | | | | 89824994, 89825067-89826365, 89826482-89828325, 89828450-89833442, 89833513-89834842, 89834946- |
| | | | | 89835917, 89836096-89836171, 89836295-89837062, 89837229-89838144, 89838326-89843029, 89843198- |
| | | | | 89843529, 89843617-89844555, 89844674-89847093, 89847147-89848344, 89848474-89848570, 89848641- |
| | | | | 89848834, 89848930-89849236, 89849426-89850705, 89850761-89850842, 89850904-89856133, 89856204- |
| | | | | 89857841, 89857939-89858511, 89858621-89859626, |
| 40 | FANCI | NM_018193 | chr15 | 89787265-89790858, 89790963-89801933, 89802008-89803942, 89804075-89804814, 89804973- |
| | | | | 89805050, 89805110-89806648, 89806692-89807132, 89807258-89807751, 89807839-89811628, 89811757- |
| | | | | 89816606, 89816701-89817397, 89817536-89819940, 89820123-89821916, 89822006-89824399, 89824532- |
| | | | | 89824994, 89825067-89826365, 89826482-89828325, 89828450-89833442, 89833513-89834842, 89834946- |
| | | | | 89835917, 89836096-89836171, 89836295-89837062, 89837229-89843029, 89843198-89843529, 89843617- |
| | | | | 89844555, 89844674-89847093, 89847147-89848344, 89848474-89848570, 89848641-89848834, 89848930- |
| | | | | 89849236, 89849426-89850705, 89850761-89850842, 89850904-89856133, 89856204-89857841, 89857939- |
| | | | | 89858511, 89858621-89859626, |
| 40 | RHCG | NM_016321 | chr15 | 90015087-90015940, 90016095-90019984, 90020060-90020311, 90020438-90020746, 90020885- |
| | | | | 90021066, 90021206-90022551, 90022720-90023490, 90023640-90026296, 90026449-90030028, 90030217- |
| | | | | 90039590, |
| 40 | LOC283761 | NR_027076 | chr15 | 90048839-90053302, 90053355-90066810, |
| 40 | LOC283761 | NR_027075 | chr15 | 90049090-90053302, 90053355-90060631, 90060669-90066810, |
| 40 | LOC283761 | NR_027074 | chr15 | 90050008-90053302, 90053355-90066810, |
| 40 | LOC283761 | NR_027077 | chr15 | 90049010-90053302, 90053355-90066810, |
| 40 | C15orf42 | NM_152259 | chr15 | 90119472-90125915, 90126197-90127515, 90127759-90128937, 90129174-90135276, 90135408- |
| | | | | 90137644, 90137786-90138622, 90138843-90142553, 90142707-90143814, 90143917-90144527, 90144623- |
| | | | | 90144718, 90144800-90144966, 90145208-90146025, 90146124-90149997, 90150057-90152032, 90152181- |
| | | | | 90159634, 90159727-90161381, 90161442-90162937, 90163074-90164622, 90164782-90166852, 90169018- |
| | | | | 90169165, 90169293-90170185, |
| 40 | ZNF710 | NM_198526 | chr15 | 90544847-90610340, 90611828-90616301, 90616495-90617346, 90617523-90622890, |
| 40 | IDH2 | NM_002168 | chr15 | 90627586-90628046, 90628141-90628231, 90628331-90628505, 90628620-90630342, 90630496- |
| | | | | 90630669, 90630808-90631589, 90631735-90631817, 90631980-90633709, 90633877-90634783, 90634877- |
| | | | | 90645506, |
| 40 | SEMA4B | NM_198925 | chr15 | 90744968-90760669, 90760835-90760997, 90761062-90763023, 90763124-90764212, 90764326- |
| | | | | 90764597, 90764713-90764844, 90764998-90766789, 90766973-90767068, 90767221-90768198, 90768411- |
| | | | | 90768512, 90768630-90768891, 90769060-90770787, 90770875-90771134, |
| 40 | SEMA4B | NM_020210 | chr15 | 90728314-90744688, 90744968-90760669, 90760835-90760997, 90761062-90763023, 90763124- |
| | | | | 90764212, 90764326-90764597, 90764713-90764844, 90764998-90766789, 90766973-90767068, 90767221- |
| | | | | 90768198, 90768411-90768512, 90768630-90768891, 90769060-90770787, 90770875-90771134, |
| 40 | NGRN | NM_001033088 | chr15 | 90809109-90809518, 90809631-90814418, |
| 40 | NGRN | NR_028052 | chr15 | 90809631-90814418, |
| 40 | BLM | NM_000057 | chr15 | 91260672-91290617, 91290721-91292595, 91293298-91295015, 91295177-91298039, 91298169- |
| | | | | 91303375, 91303510-91303822, 91304486-91306194, 91306388-91308524, 91308645-91310138, 91310254- |
| | | | | 91312361, 91312462-91312666, 91312817-91326050, 91326159-91328149, 91328312-91333877, 91334075- |
| | | | | 91337395, 91337588-91341418, 91341568-91346749, 91346951-91347395, 91347590-91352365, 91352490- |
| | | | | 91354433, 91354637-91358330, |
| 40 | PRC1 | NM_003981 | chr15 | 91510433-91512307, 91512351-91512675, 91512854-91513632, 91513745-91517364, 91517477- |
| | | | | 91517813, 91517962-91519904, 91520002-91522386, 91522525-91523470, 91523620-91524112, 91524264- |
| | | | | 91524727, 91524900-91524976, 91525212-91527246, 91527371-91527921, 91528056-91537635, |
| 40 | PRC1 | NM_199414 | chr15 | 91510433-91512752, 91512854-91513632, 91513745-91517364, 91517477-91517813, 91517962- |
| | | | | 91519904, 91520002-91522386, 91522525-91523470, 91523620-91524112, 91524264-91524727, 91524900- |
| | | | | 91524976, 91525212-91527246, 91527371-91527921, 91528056-91537635, |
| 40 | PRC1 | NM_199413 | chr15 | 91510433-91512675, 91512854-91513632, 91513745-91517364, 91517477-91517813, 91517962- |
| | | | | 91519904, 91520002-91522386, 91522525-91523470, 91523620-91524112, 91524264-91524727, 91524900- |
| | | | | 91524976, 91525212-91527246, 91527371-91527921, 91528056-91537635, |
| 40 | LRRC28 | NM_144598 | chr15 | 99791747-99796101, 99796331-99816779, 99816822-99827460, 99827500-99828017, 99828157- |
| | | | | 99874126, 99874335-99892572, 99892677-99901539, 99901717-99903309, 99903471-99926233, |
| 41 | CNGB3 | NM_019098 | chr8 | 87588359-87590915, 87591092-87591332, 87591481-87616319, 87616440-87623814, 87623900- |
| | | | | 87638209, 87638309-87641145, 87641307-87644978, 87645122-87655977, 87656102-87656848, 87656915- |
| | | | | 87660027, 87660116-87666238, 87666291-87679151, 87679362-87680245, 87680397-87683170, 87683327- |
| | | | | 87738757, 87738886-87751881, 87751965-87755725, |
| 41 | RIPK2 | NM_003821 | chr8 | 90770462-90775055, 90775211-90777567, 90777725-90781998, 90782158-90783967, 90784019- |
| | | | | 90784893, 90785057-90792301, 90792389-90796276, 90796368-90798819, 90798915-90801547, 90801711- |
| | | | | 90802305, |
| 41 | LRRC69 | NM_001129890 | chr8 | 92115073-92136719, 92136848-92139314, 92139389-92145336, 92145534-92147894, 92147968- |
| | | | | 92201748, 92201852-92212839, 92213021-92231116, |
| 41 | RAD54B | NM_012415 | chr8 | 95384616-95390406, 95390609-95390794, 95390863-95392371, 95392635-95399210, 95399388- |
| | | | | 95403835, 95404128-95405969, 95406111-95411640, 95411850-95412464, 95412692-95416303, 95416468- |
| | | | | 95419665, 95419949-95423347, 95423544-95470494, 95470665-95479631, 95479784-95487227, |
| 41 | ESRP1 | NM_001122827 | chr8 | 95653679-95654182, 95654313-95655529, 95655645-95658394, 95658511-95674498, 95674599- |
| | | | | 95674727, 95674784-95676923, 95677036-95677153, 95677288-95677380, 95677425-95680175, 95680479- |
| | | | | 95683679, 95683900-95686534, 95686723-95690429, 95690600-95718151, |
| 41 | ESRP1 | NM_017697 | chr8 | 95653679-95654182, 95654313-95655529, 95655645-95658394, 95658511-95674498, 95674599- |
| | | | | 95674727, 95674784-95676923, 95677036-95677153, 95677288-95677380, 95677425-95680175, 95680479- |
| | | | | 95683679, 95683900-95686534, 95686735-95690429, 95690600-95704903, 95705056-95709078, 95709190- |
| | | | | 95718151, |
| 41 | ESRP1 | NM_001122825 | chr8 | 95653679-95654182, 95654313-95655529, 95655645-95658394, 95658511-95674498, 95674599- |
| | | | | 95674727, 95674784-95676923, 95677036-95677153, 95677288-95677380, 95677425-95680175, 95680479- |
| | | | | 95683679, 95683900-95686534, 95686735-95690429, 95690600-95718151, |
| 41 | ESRP1 | NM_001122826 | chr8 | 95653679-95654182, 95654313-95655529, 95655645-95658394, 95658511-95674498, 95674599- |
| | | | | 95674727, 95674784-95676923, 95677036-95677153, 95677288-95677380, 95677425-95680175, 95680479- |
| | | | | 95683679, 95683900-95686534, 95686723-95690429, 95690600-95704903, 95705056-95718151, |
| 41 | ESRP1 | NM_001034915 | chr8 | 95653679-95654182, 95654313-95655529, 95655645-95658394, 95658511-95674498, 95674599- |
| | | | | 95674727, 95674784-95676923, 95677036-95677153, 95677288-95677380, 95677425-95680175, 95680479- |
| | | | | 95683679, 95683900-95686534, 95686723-95690429, 95690600-95704903, 95705056-95709078, 95709190- |
| | | | | 95718151, |
| 41 | UQCRB | NM_006294 | chr8 | 97243361-97244000, 97244169-97245382, 97245456-97247738, |
| 41 | UQCRB | NM_001199975 | chr8 | 97243361-97244000, 97244169-97245382, 97245456-97246649, 97246765-97247738, |
| 41 | PTDSS1 | NM_014754 | chr8 | 97274448-97285525, 97285619-97296335, 97296382-97299248, 97299375-97307324, 97307485- |
| | | | | 97311920, 97312074-97316266, 97316410-97318670, 97318785-97321783, 97321851-97332472, 97332574- |
| | | | | 97342439, 97342510-97343252, 97343324-97345683, |
| 41 | STK3 | NM_006281 | chr8 | 99468229-99538968, 99539146-99560195, 99560390-99591890, 99592018-99608258, 99608398- |
| | | | | 99718693, 99718863-99719373, 99719540-99761502, 99761619-99779469, 99779600-99786965, 99787048- |
| | | | | 99837741, |
| 41 | VPS13B | NM_152564 | chr8 | 100025576-100025986, 100026164-100050649, 100050795-100108538, 100108661- |
| | | | | 100115179, 100115349-100123324, 100123508-100127926, 100128103-100133403, 100133674- |
| | | | | 100146858, 100146956-100147241, 100147366-100147822, 100147962-100148891, 100148981- |
| | | | | 100155200, 100155394-100160067, 100160239-100168775, 100168972-100182265, 100182392- |
| | | | | 100205102, 100205286-100286424, 100286561-100287307, 100287483-100396434, 100396546- |
| | | | | 100403783, 100403933-100443763, 100443893-100454627, 100454864-100479640, 100479863- |
| | | | | 100493825, 100494031-100513913, 100514087-100515062, 100515179-100519363, 100519432- |
| | | | | 100523330, 100523741-100533125, 100533239-100568676, 100568882-100587884, 100588013- |
| | | | | 100589716, 100589862-100654037, 100654727-100673580, 100673720-100711751, 100712161- |
| | | | | 100729397, 100729602-100732571, 100732781-100733089, 100733276-100779000, 100779199- |
| | | | | 100789001, 100789185-100790908, 100791260-100796541, 100796705-100821601, 100821759- |
| | | | | 100829766, 100830032-100830677, 100830763-100830939, 100831117-100831638, 100831811- |
| | | | | 100832147, 100832351-100833520, 100833711-100836058, 100836207-100844595, 100844881- |
| | | | | 100847423, 100847553-100847765, 100847967-100861002, 100861123-100865677, 100866485- |
| | | | | 100871530, 100871709-100874002, 100874175-100880515, 100880694-100883011, 100883116- |
| | | | | 100883674, 100883926-100887644, |
| 41 | VPS13B | NM_181661 | chr8 | 100025576-100025986, 100026164-100050649, 100050795-100108538, 100108661- |
| | | | | 100115179, 100115349-100123324, 100123508-100127926, 100128103-100133403, |
| 41 | VPS13B | NM_017890 | chr8 | 100025576-100025986, 100026164-100050649, 100050795-100108538, 100108661- |
| | | | | 100115179, 100115349-100123324, 100123508-100127926, 100128103-100133403, 100133674- |
| | | | | 100146858, 100146956-100147241, 100147366-100147822, 100147962-100148891, 100148981- |
| | | | | 100155200, 100155394-100160067, 100160239-100168775, 100168972-100182265, 100182392- |
| | | | | 100205102, 100205286-100286424, 100286561-100287307, 100287483-100396434, 100396546- |
| | | | | 100403783, 100403933-100443763, 100443893-100454627, 100454864-100479640, 100479863- |
| | | | | 100493825, 100494031-100513913, 100514087-100515062, 100515179-100519996, 100520140- |
| | | | | 100523330, 100523741-100533125, 100533239-100568676, 100568882-100587884, 100588013- |
| | | | | 100589716, 100589862-100654037, 100654727-100673580, 100673720-100711751, 100712161- |
| | | | | 100729397, 100729602-100732571, 100732781-100733089, 100733276-100779000, 100779199- |
| | | | | 100789001, 100789185-100790908, 100791260-100796541, 100796705-100821601, 100821759- |
| | | | | 100829766, 100830032-100830677, 100830763-100830939, 100831117-100831638, 100831811- |
| | | | | 100832147, 100832351-100833520, 100833711-100836058, 100836207-100844595, 100844881- |
| | | | | 100847423, 100847553-100847765, 100847967-100861002, 100861123-100865677, 100866485- |
| | | | | 100871530, 100871709-100874002, 100874175-100880515, 100880694-100883011, 100883116- |
| | | | | 100883674, 100883926-100887644, |
| 41 | VPS13B | NM_015243 | chr8 | 100025576-100025986, 100026164-100050649, 100050795-100108538, 100108661- |
| | | | | 100115179, 100115349-100123324, 100123508-100127926, 100128103-100133403, 100133674- |
| | | | | 100146858, 100146956-100147241, 100147366-100147822, 100147962-100148891, 100148981- |
| | | | | 100155200, 100155394-100160067, 100160239-100168775, 100168972-100182265, 100182392- |
| | | | | 100205102, 100205286-100221818, |
| 41 | RNF19A | NM_015435 | chr8 | 101271475-101272080, 101272226-101273768, 101273984-101276260, 101276424- |
| | | | | 101276897, 101277014-101281011, 101281176-101282095, 101282242-101287179, 101287390- |
| | | | | 101299727, 101300496-101312754, 101312880-101315386, |
| 41 | RNF19A | NM_183419 | chr8 | 101271475-101272080, 101272226-101273768, 101273984-101276260, 101276424- |
| | | | | 101276897, 101277014-101281011, 101281176-101282095, 101282242-101287179, 101287390- |
| | | | | 101299727, 101300496-101322093, |
| 41 | PABPC1 | NM_002568 | chr8 | 101715588-101716523, 101716619-101717152, 101717285-101717815, 101717902- |
| | | | | 101718877, 101719034-101719113, 101719226-101721359, 101721452-101721685, 101721960- |
| | | | | 101724588, 101724686-101724878, 101725018-101725313, 101725410-101727688, 101727830- |
| | | | | 101729999, 101730117-101730313, 101730509-101733617, |
| 41 | YWHAZ | NM_001135699 | chr8 | 101932981-101936181, 101936279-101936361, 101936527-101937142, 101937268- |
| | | | | 101960822, 101961129-101965076, |
| 41 | YWHAZ | NM_001135701 | chr8 | 101932981-101936181, 101936279-101936361, 101936527-101937142, 101937268- |
| | | | | 101960822, 101961129-101963412, |
| 41 | YWHAZ | NM_001135702 | chr8 | 101932981-101936181, 101936279-101936361, 101936527-101937142, 101937268- |
| | | | | 101960822, 101961129-101962632, |
| 41 | YWHAZ | NM_001135700 | chr8 | 101932981-101936181, 101936279-101936361, 101936527-101937142, 101937268- |
| | | | | 101960822, 101961129-101963461, |
| 41 | YWHAZ | NM_145690 | chr8 | 101932981-101936181, 101936279-101936361, 101936527-101937142, 101937268- |
| | | | | 101960822, 101961129-101964155, |
| 41 | YWHAZ | NM_003406 | chr8 | 101932981-101936181, 101936279-101936361, 101936527-101937142, 101937268- |
| | | | | 101960822, 101961129-101965495, |
| 41 | FLJ42969 | NR_033962 | chr8 | 102065065-102066724, 102066808-102087395, |
| 41 | GRHL2 | NM_024915 | chr8 | 102505018-102555467, 102555665-102564941, 102565011-102570645, 102571041- |
| | | | | 102582565, 102582623-102585894, 102586053-102589634, 102589748-102611283, 102611380- |
| | | | | 102631765, 102631926-102643863, 102643953-102644452, 102644594-102649123, 102649157- |
| | | | | 102656357, 102656454-102661640, 102661728-102676680, 102676747-102678815, |
| 41 | NCALD | NM_001040629 | chr8 | 102701635-102705017, 102705125-102731478, 102731877-102792150, 102792208- |
| | | | | 102928035, 102928087-103032463, 103032518-103136463, |
| 41 | NCALD | NM_001040630 | chr8 | 102701635-102705017, 102705125-102731478, 102731877-102928035, 102928087- |
| | | | | 103032463, 103032518-103136796, |
| 41 | NCALD | NM_001040627 | chr8 | 102701635-102705017, 102705125-102731478, 102731877-102928035, 102928087- |
| | | | | 103032463, 103032518-103136463, |
| 41 | NCALD | NM_032041 | chr8 | 102701635-102705017, 102705125-102731478, 102731877-102803088, |
| 41 | NCALD | NM_001040628 | chr8 | 102701635-102705017, 102705125-102731478, 102731877-102792150, 102792208- |
| | | | | 102928035, 102928087-103032463, 103032518-103041236, 103041325-103136463, |
| 41 | NCALD | NM_001040626 | chr8 | 102701635-102705017, 102705125-102731478, 102731877-102899367, 102899456- |
| | | | | 102928035, 102928087-103032463, 103032518-103136463, |
| 41 | NCALD | NM_001040625 | chr8 | 102701635-102705017, 102705125-102731478, 102731877-102928035, 102928087- |
| | | | | 103032463, 103032518-103041236, 103041325-103136463, |
| 41 | NCALD | NM_001040624 | chr8 | 102701635-102705017, 102705125-102731478, 102731877-102899367, 102899456- |
| | | | | 102928035, 102928087-103032463, 103032518-103041236, 103041325-103136463, |
| 41 | AZIN1 | NM_015878 | chr8 | 103840907-103841498, 103841715-103842047, 103842165-103845282, 103845447- |
| | | | | 103846415, 103846492-103846892, 103846976-103848482, 103848619-103850970, 103851145- |
| | | | | 103851876, 103852052-103855777, 103855976-103862225, 103862373-103870239, 103870379- |
| | | | | 103876033, |
| 41 | AZIN1 | NM_148174 | chr8 | 103840907-103841498, 103841715-103842047, 103842165-103845282, 103845447- |
| | | | | 103846415, 103846492-103846892, 103846976-103848482, 103848619-103850970, 103851145- |
| | | | | 103851876, 103852052-103855777, 103855976-103870239, 103870379-103876033, |
| 41 | SLC25A32 | NM_030780 | chr8 | 104412775-104413742, 104413890-104414167, 104414283-104415390, 104415553- |
| | | | | 104417002, 104417090-104419860, 104420013-104427010, |
| 41 | LRP12 | NM_001135703 | chr8 | 105503768-105507303, 105507438-105509198, 105510305-105511543, 105511748- |
| | | | | 105521165, 105521303-105601045, |
| 41 | LRP12 | NM_013437 | chr8 | 105503768-105507303, 105507438-105509198, 105510305-105511543, 105511748- |
| | | | | 105521165, 105521303-105544133, 105544192-105601045, |
| 41 | OXR1 | NM_001198533 | chr8 | 107282596-107371702, 107371865-107531169, 107531368-107691436, 107691521- |
| | | | | 107695425, 107695535-107696472, 107696588-107704954, 107705106-107715132, 107715319- |
| | | | | 107718608, 107719374-107722848, 107723019-107726049, 107726214-107749746, 107749829- |
| | | | | 107751684, 107751812-107752569, 107752724-107754448, 107754546-107758015, 107758091- |
| | | | | 107763032, |
| 41 | OXR1 | NM_001198534 | chr8 | 107738538-107749746, 107749829-107751684, 107751812-107752569, 107752724- |
| | | | | 107754448, 107754546-107758015, 107758091-107763032, |
| 41 | OXR1 | NM_181354 | chr8 | 107670414-107691436, 107691521-107695425, 107695535-107696472, 107696588- |
| | | | | 107704954, 107705106-107715132, 107715319-107718608, 107719374-107722848, 107723019- |
| | | | | 107726049, 107726214-107751684, 107751812-107752569, 107752724-107754448, 107754546- |
| | | | | 107758015, 107758091-107763032, |
| 41 | OXR1 | NM_018002 | chr8 | 107282596-107371702, 107371865-107531169, 107531368-107691436, 107691521- |
| | | | | 107695425, 107695535-107696472, 107696588-107704954, 107705106-107715132, 107715319- |
| | | | | 107718608, 107719374-107722848, 107723019-107726049, 107726214-107751684, 107751812- |
| | | | | 107752569, 107752724-107754448, 107754546-107758015, 107758091-107763032, |
| 41 | OXR1 | NM_001198532 | chr8 | 107460277-107531169, 107531368-107691436, 107691521-107695425, 107695535- |
| | | | | 107696472, 107696588-107704954, 107705106-107715132, 107715319-107718608, 107719374- |
| | | | | 107722848, 107723019-107726049, 107726214-107749746, 107749829-107751684, 107751812- |
| | | | | 107752569, 107752724-107754448, 107754546-107758015, 107758091-107763032, |
| 41 | OXR1 | NM_001198535 | chr8 | 107738538-107751684, 107751812-107752569, 107752724-107754448, 107754546- |
| | | | | 107758015, 107758091-107763032, |
| 41 | EIF3E | NM_001568 | chr8 | 109214153-109215210, 109215347-109215628, 109215733-109226834, 109226946- |
| | | | | 109228639, 109228743-109229561, 109229690-109240494, 109240621-109241297, 109241425- |
| | | | | 109247225, 109247332-109248388, 109248433-109252185, 109252305-109254026, 109254143- |
| | | | | 109260840, |
| 41 | EIF3H | NM_003756 | chr8 | 117657343-117658708, 117658843-117661043, 117661166-117668093, 117668245- |
| | | | | 117669452, 117669554-117671050, 117671220-117738253, 117738412-117767903, |
| 41 | EXT1 | NM_000127 | chr8 | 118812137-118816959, 118817133-118819454, 118819617-118825109, 118825201- |
| | | | | 118830672, 118830770-118831913, 118832034-118834702, 118834837-118842467, 118842589- |
| | | | | 118847681, 118847791-118849345, 118849441-119122322, |
| 41 | TAF2 | NM_003184 | chr8 | 120744427-120754772, 120754897-120756526, 120756634-120758943, 120759175- |
| | | | | 120768244, 120768356-120770311, 120770383-120772837, 120772979-120774653, 120774849- |
| | | | | 120790257, 120790370-120793291, 120793434-120795619, 120795874-120797439, 120797507- |
| | | | | 120800576, 120800688-120801028, 120801145-120801830, 120801987-120803562, 120803700- |
| | | | | 120805604, 120805692-120807770, 120807872-120809228, 120809344-120809900, 120810087- |
| | | | | 120814032, 120814266-120816116, 120816260-120818521, 120818642-120831584, 120831747- |
| | | | | 120843915, 120843972-120844720, |
| 41 | MTBP | NM_022045 | chr8 | 121457829-121458671, 121458754-121463241, 121463317-121463409, 121463563- |
| | | | | 121466085, 121466146-121467673, 121467820-121468791, 121468911-121471453, 121471590- |
| | | | | 121473399, 121473496-121475930, 121476002-121483058, 121483178-121500397, 121500573- |
| | | | | 121502701, 121502811-121509631, 121509795-121514730, 121514850-121518944, 121519102- |
| | | | | 121522172, 121522270-121528163, 121528432-121530089, 121530341-121530942, 121531058- |
| | | | | 121534892, 121534960-121535536, |
| 41 | WDR67 | NM_145647 | chr8 | 124085087-124089349, 124089498-124094940, 124095058-124096400, 124096581- |
| | | | | 124105829, 124105983-124109520, 124109682-124113045, 124113248-124117526, 124117705- |
| | | | | 124121555, 124121637-124121712, 124121861-124132293, 124132429-124138294, 124138430- |
| | | | | 124138746, 124138928-124140519, 124140754-124141304, 124141459-124142436, 124142574- |
| | | | | 124146352, 124146447-124152999, 124153142-124154500, 124154697-124156955, 124157096- |
| | | | | 124162274, 124162369-124164044, |
| 41 | WDR67 | NM_001145088 | chr8 | 124085087-124089349, 124089498-124094940, 124095058-124096400, 124096581- |
| | | | | 124105829, 124105983-124109520, 124109682-124113045, 124113248-124117526, 124117705- |
| | | | | 124121555, 124121637-124121712, 124121861-124132293, 124132429-124138294, 124138430- |
| | | | | 124138746, 124138928-124140519, 124140754-124141304, 124141459-124142436, 124142574- |
| | | | | 124152999, 124153142-124156955, 124157096-124162274, 124162369-124164044, |
| 41 | FAM83A | NM_032899 | chr8 | 124195577-124204041, 124204211-124206262, 124206389-124219395, |
| 41 | FAM83A | NM_207006 | chr8 | 124195577-124204041, 124204211-124206262, 124206389-124219395, 124219657-124221664, |
| 41 | NDRG1 | NM_006096 | chr8 | 134251363-134254264, 134254318-134256596, 134256634-134258857, 134258907- |
| | | | | 134260116, 134260170-134260956, 134261015-134262681, 134262787-134266780, 134266839- |
| | | | | 134269018, 134269107-134270607, 134270670-134271409, 134271474-134274288, 134274411- |
| | | | | 134276788, 134276896-134292473, 134292511-134296490, 134296573-134309375, |
| 41 | NDRG1 | NM_001135242 | chr8 | 134251363-134254264, 134254318-134256596, 134256634-134258857, 134258907- |
| | | | | 134260116, 134260170-134260956, 134261015-134262681, 134262787-134266780, 134266839- |
| | | | | 134269018, 134269107-134270607, 134270670-134271409, 134271474-134274288, 134274411- |
| | | | | 134276788, 134276896-134292473, 134292511-134296490, 134296573-134308978, |
| 41 | PTK2 | NM_005607 | chr8 | 141669778-141675014, 141675097-141678366, 141678524-141684395, 141684504- |
| | | | | 141685557, 141685599-141696729, 141696794-141710988, 141711122-141712667, 141712807- |
| | | | | 141716216, 141716305-141727695, 141727809-141745348, 141745555-141749115, 141749207- |
| | | | | 141753327, 141753430-141754749, 141754867-141756857, 141756960-141762329, 141762416- |
| | | | | 141771262, 141771362-141774331, 141774390-141799571, 141799626-141803451, 141803484- |
| | | | | 141810556, 141810676-141813640, 141813750-141828374, 141828454-141828977, 141829120- |
| | | | | 141840569, 141840626-141856357, 141856422-141856696, 141856778-141874409, 141874499- |
| | | | | 141889568, 141889737-141900640, 141900869-141994175, 141994255-142011222, |
| 41 | PTK2 | NM_153831 | chr8 | 141669778-141675014, 141675097-141678366, 141678524-141684395, 141684504- |
| | | | | 141685557, 141685599-141696729, 141696794-141710988, 141711122-141712667, 141712807- |
| | | | | 141716216, 141716305-141727695, 141727809-141745348, 141745555-141749115, 141749207- |
| | | | | 141753327, 141753430-141754749, 141754867-141756857, 141756960-141762329, 141762416- |
| | | | | 141771262, 141771362-141774331, 141774390-141799571, 141799626-141803451, 141803484- |
| | | | | 141810556, 141810676-141813640, 141813750-141828374, 141828454-141828977, 141829120- |
| | | | | 141840569, 141840626-141856357, 141856422-141856696, 141856778-141874409, 141874499- |
| | | | | 141889568, 141889737-141900640, 141900869-141935758, 141935849-142011222, |
| 41 | PTK2 | NM_001199649 | chr8 | 141669778-141675014, 141675097-141678366, 141678524-141679824, 141679835- |
| | | | | 141684395, 141684504-141685527, 141685599-141696729, 141696794-141710988, 141711122- |
| | | | | 141712667, 141712807-141716216, 141716305-141727695, 141727809-141745348, 141745555- |
| | | | | 141749115, 141749207-141753327, 141753430-141754749, 141754867-141756857, 141756960- |
| | | | | 141762329, 141762416-141771262, 141771362-141774331, 141774390-141799571, 141799626- |
| | | | | 141803451, 141803484-141810556, 141810676-141813640, 141813750-141828374, 141828454- |
| | | | | 141828977, 141829120-141840569, 141840626-141856357, 141856422-141856696, 141856778- |
| | | | | 141874409, 141874499-141889568, 141889737-141900640, 141900869-141935758, 141935849- |
| | | | | 142011222, |
| 41 | SLURP1 | NM_020427 | chr8 | 143822695-143823219, 143823341-143823744, |
| 41 | LYPD2 | NM_205545 | chr8 | 143831901-143832467, 143832589-143833810, |
| 41 | LY6D | NM_003695 | chr8 | 143866873-143867003, 143867104-143867879, |
| 41 | LY6H | NM_002347 | chr8 | 144239903-144240218, 144240340-144240997, 144241127-144241351, |
| 41 | LY6H | NM_001135655 | chr8 | 144239903-144240218, 144240340-144240997, 144241127-144241613, |
| 41 | LY6H | NM_001130478 | chr8 | 144239903-144240218, 144240340-144240997, 144241127-144241613, 144241685-144241961, |
| 41 | C8orf73 | NM_001100878 | chr8 | 144649636-144649779, 144649847-144649954, 144650059-144650310, 144650432- |
| | | | | 144650720, 144650890-144651609, 144651785-144651865, 144652011-144652119, 144652236- |
| | | | | 144652411, 144652551-144652641, 144652828-144653045, 144653165-144653835, 144653992- |
| | | | | 144654201, 144654356-144654589, |
| 41 | SPATC1 | NM_198572 | chr8 | 145086895-145094808, 145095365-145095467, 145096009-145096131, 145096273-145101526, |
| 41 | SPATC1 | NM_001134374 | chr8 | 145086895-145094808, 145095365-145095467, 145096009-145101526, |
| 41 | HEATR7A | NM_032450 | chr8 | 145202980-145215871, 145215993-145218672, 145218752-145223196, 145223344- |
| | | | | 145234356, 145234490-145235079, 145235244-145235326, 145235427-145245685, 145245839- |
| | | | | 145246616, 145246759-145247210, 145247305-145254023, 145254104-145255329, 145255445- |
| | | | | 145275501, 145275577-145278009, 145278134-145293657, 145293767-145293936, 145294083- |
| | | | | 145294224, 145294267-145294515, 145294659-145295002, 145295057-145295471, 145295581- |
| | | | | 145295893, 145296015-145296296, 145296421-145297270, 145297419-145297503, 145297532- |
| | | | | 145298395, 145298520-145298764, 145298846-145299123, 145299240-145299345, 145299443- |
| | | | | 145300057, 145300164-145302202, 145302340-145302468, 145302583-145303778, 145303933- |
| | | | | 145305113, 145305270-145309714, 145309882-145310410, 145310609-145313678, 145313818- |
| | | | | 145314141, 145314258-145314812, 145314961-145315087, 145315278-145315578, 145315736- |
| | | | | 145315808, 145315945-145316015, 145316120-145316185, 145316253-145316556, |
| 41 | HEATR7A | NM_001099281 | chr8 | 145202980-145218672, 145218752-145223196, 145223344-145234356, 145234490- |
| | | | | 145235079, 145235244-145235326, 145235427-145245685, 145245839-145246616, 145246759- |
| | | | | 145247210, 145247305-145254023, 145254104-145255329, 145255445-145267895, |
| 41 | HEATR7A | NM_001099280 | chr8 | 145202980-145215871, 145215993-145218672, 145218752-145223196, 145223344- |
| | | | | 145234356, 145234490-145235079, 145235244-145235326, 145235427-145245685, 145245839- |
| | | | | 145246616, 145246759-145247210, 145247305-145254023, 145254104-145255329, 145255445- |
| | | | | 145267895, |
| 41 | ADCK5 | NM_174922 | chr8 | 145597797-145603074, 145603180-145608280, 145608432-145615869, 145615947- |
| | | | | 145616054, 145616257-145616332, 145616475-145616559, 145616675-145616778, 145616912- |
| | | | | 145616981, 145617067-145617139, 145617223-145617290, 145617371-145617452, 145617546- |
| | | | | 145617658, 145617913-145617989, 145618108-145618182, |
| 41 | CYHR1 | NM_001129888 | chr8 | 145689996-145690190, 145690480-145690952, |
| 41 | CYHR1 | NM_138496 | chr8 | 145676154-145677740, 145677954-145678603, 145678844-145689841, 145689996-145690190, |
| 41 | CYHR1 | NM_032687 | chr8 | 145689996-145690190, 145690480-145690734, |
| 41 | FOXH1 | NM_003923 | chr8 | 145700440-145700538, 145700645-145700964, |
| 42 | CREBL2 | NM_001310 | chr12 | 12765122-12788709, 12788909-12790502, 12790649-12794926, |
| 42 | DDX47 | NM_016355 | chr12 | 12966389-12967063, 12967159-12974140, 12974331-12974587, 12974661-12974897, 12975018- |
| | | | | 12975593, 12975667-12976135, 12976254-12976802, 12976951-12977472, 12977612-12978932, 12979005- |
| | | | | 12980178, 12980310-12982355, |
| 42 | DDX47 | NM_201224 | chr12 | 12966389-12967063, 12967159-12974140, 12974331-12974587, 12974661-12974897, 12975018- |
| | | | | 12975593, 12975667-12976135, 12976254-12977472, 12977612-12978932, 12979005-12980178, 12980310- |
| | | | | 12982355, |
| 42 | HEBP1 | NM_015987 | chr12 | 13128414-13140084, 13140267-13142209, 13142350-13152969, |
| 42 | RERG | NM_032918 | chr12 | 15262452-15264276, 15264352-15273995, 15274054-15370361, 15370538-15374127, |
| 42 | RERG | NM_001190726 | chr12 | 15262452-15264276, 15264352-15370361, 15370538-15374127, |
| 42 | DERA | NM_015954 | chr12 | 16064349-16109868, 16109968-16111120, 16111270-16112761, 16112859-16115745, 16115882- |
| | | | | 16135240, 16135371-16185474, 16185589-16189164, 16189316-16189622, |
| 42 | KIAA0528 | NM_014802 | chr12 | 22602826-22606828, 22606977-22609903, 22610096-22622641, 22622730-22623756, 22623846- |
| | | | | 22624331, 22624423-22624899, 22625000-22625218, 22625282-22625676, 22625742-22627683, 22627826- |
| | | | | 22631245, 22631412-22635489, 22635678-22637629, 22637823-22643055, 22643153-22646155, 22646272- |
| | | | | 22659643, 22659754-22666226, 22666314-22670918, 22671072-22676358, 22676559-22677404, 22677562- |
| | | | | 22678542, 22678640-22680653, 22680827-22688190, 22688279-22696993, 22697114-22697252, |
| 42 | CASC1 | NM_018272 | chr12 | 25261758-25263059, 25263175-25264704, 25264874-25267588, 25267805-25272077, 25272236- |
| | | | | 25274685, 25274848-25276143, 25276221-25297297, 25297612-25299933, 25300039-25302560, 25302709- |
| | | | | 25307251, 25307391-25308243, 25308347-25311404, 25311498-25314045, 25314132-25343546, 25343566- |
| | | | | 25348008, |
| 42 | CASC1 | NM_001082972 | chr12 | 25261696-25263059, 25263175-25264704, 25264874-25267588, 25267805-25272077, 25272236- |
| | | | | 25274685, 25274848-25276143, 25276221-25297297, 25297612-25299933, 25300039-25302560, 25302709- |
| | | | | 25307251, 25307391-25308243, 25308347-25311404, 25311498-25314045, 25314132-25343546, 25343566- |
| | | | | 25347818, |
| 42 | CASC1 | NM_001082973 | chr12 | 25261758-25263059, 25263175-25264704, 25264874-25267588, 25267805-25272077, 25272236- |
| | | | | 25274685, 25274848-25276143, 25276221-25297297, 25297612-25299933, 25300039-25302560, 25302709- |
| | | | | 25307251, 25307391-25308243, 25308347-25311404, 25311498-25314045, 25314132-25348008, |
| 42 | AMN1 | NM_001113402 | chr12 | 31825312-31841939, 31842053-31850293, 31850352-31850672, 31850892-31854795, 31854942- |
| | | | | 31862225, 31862360-31881903, |
| 42 | AMN1 | NR_004854 | chr12 | 31825312-31841939, 31842053-31850293, 31850352-31850672, 31850892-31854795, 31854942- |
| | | | | 31862225, 31862360-31881923, |
| 42 | C12orf35 | NM_018169 | chr12 | 32112426-32113709, 32113806-32123121, 32123291-32133810, 32138892-32140171, 32140257-32145310, |
| 43 | SMPDL3A | NM_006714 | chr6 | 123110604-123116820, 123117036-123117967, 123118114-123122453, 123122552- |
| | | | | 123124807, 123124979-123126052, 123126235-123127376, 123127503-123130234, |
| 43 | CENPW | NM_001012507 | chr6 | 126661546-126667349, 126667465-126669610, |
| 43 | C6orf58 | NM_001010905 | chr6 | 127898632-127899829, 127899918-127901408, 127901595-127902325, 127902428- |
| | | | | 127911230, 127911471-127912686, |
| 43 | PTPRK | NM_002844 | chr6 | 128291426-128294165, 128294303-128294807, 128294973-128297800, 128297928- |
| | | | | 128298066, 128298200-128302259, 128302435-128303974, 128304126-128304385, 128304523- |
| | | | | 128306864, 128307021-128311912, 128312031-128312437, 128312537-128313816, 128313855- |
| | | | | 128316605, 128316684-128318044, 128318134-128319863, 128320050-128326227, 128326390- |
| | | | | 128330273, 128330414-128385901, 128385940-128388662, 128388938-128399956, 128400064- |
| | | | | 128403580, 128403784-128404858, 128404970-128410833, 128411138-128505575, 128505871- |
| | | | | 128540065, 128540242-128561178, 128561296-128563664, 128563748-128643182, 128643456- |
| | | | | 128718709, 128718834-128841402, |
| 43 | PTPRK | NM_001135648 | chr6 | 128291426-128294165, 128294303-128294807, 128294973-128297800, 128297928- |
| | | | | 128298066, 128298200-128302259, 128302435-128303974, 128304126-128304385, 128304523- |
| | | | | 128306864, 128307021-128311912, 128312031-128312437, 128312537-128313816, 128313855- |
| | | | | 128316392, 128316412-128316605, 128316684-128318044, 128318134-128319863, 128320050- |
| | | | | 128326227, 128326390-128330273, 128330414-128385901, 128385940-128388662, 128388938- |
| | | | | 128399956, 128400064-128403580, 128403784-128404858, 128404970-128410833, 128411138- |
| | | | | 128505575, 128505871-128540065, 128540242-128561178, 128561296-128563664, 128563748- |
| | | | | 128643182, 128643456-128718709, 128718834-128841402, |
| 43 | MED23 | NM_004830 | chr6 | 131908987-131910603, 131910737-131911460, 131911583-131912451, 131912668- |
| | | | | 131913526, 131913601-131914144, 131914312-131915237, 131915476-131917085, 131917304- |
| | | | | 131917656, 131917829-131919425, 131919608-131919694, 131919902-131921176, 131921303- |
| | | | | 131923356, 131923522-131924168, 131924345-131925316, 131925461-131926378, 131926626- |
| | | | | 131927617, 131927765-131929066, 131929212-131931184, 131931387-131937045, 131937143- |
| | | | | 131939545, 131939660-131940965, 131941037-131941766, 131941870-131943019, 131943120- |
| | | | | 131944489, 131944603-131946003, 131946130-131948534, 131948624-131948779, 131948813- |
| | | | | 131949149, |
| 43 | MED23 | NM_015979 | chr6 | 131895436-131908847, 131908987-131910603, 131910737-131911460, 131911583- |
| | | | | 131912451, 131912668-131913526, 131913601-131914144, 131914312-131915237, 131915476- |
| | | | | 131917085, 131917304-131917656, 131917829-131919425, 131919608-131919694, 131919902- |
| | | | | 131921176, 131921303-131923356, 131923522-131924168, 131924345-131925316, 131925461- |
| | | | | 131926378, 131926626-131927617, 131927765-131929066, 131929212-131931184, 131931387- |
| | | | | 131936462, 131936482-131937045, 131937143-131939545, 131939660-131940965, 131941037- |
| | | | | 131941766, 131941870-131943019, 131943120-131944489, 131944603-131946003, 131946130- |
| | | | | 131948534, 131948624-131948779, 131948813-131949149, |
| 43 | VNN3 | NR_028290 | chr6 | 133044194-133044871, 133045047-133045743, 133046119-133047859, 133048152- |
| | | | | 133049878, 133050119-133055390, 133055523-133055617, |
| 43 | VNN3 | NR_028291 | chr6 | 133044194-133044871, 133045047-133045743, 133046119-133047859, 133048152- |
| | | | | 133049878, 133050073-133055390, 133055523-133055617, |
| 43 | LOC285735 | NR_026969 | chr6 | 133409717-133417647, 133417703-133421144, 133421324-133427232, |
| 43 | MAP7 | NM_001198617 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693764-136698891, 136699007- |
| | | | | 136704807, 136704920-136732756, 136732836-136742837, 136742938-136871478, |
| 43 | MAP7 | NM_001198616 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693764-136698891, 136699007- |
| | | | | 136709529, 136709649-136710490, 136710656-136732756, 136732836-136742837, 136742938- |
| | | | | 136871478, |
| 43 | MAP7 | NM_003980 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693764-136698891, 136699007- |
| | | | | 136704807, 136704920-136709529, 136709649-136710490, 136710656-136732756, 136732836- |
| | | | | 136742837, 136742938-136871478, |
| 43 | MAP7 | NM_001198611 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693764-136698891, 136699007- |
| | | | | 136709529, 136709649-136710490, 136710656-136732756, 136732836-136742837, 136742938- |
| | | | | 136846968, |
| 43 | MAP7 | NM_001198615 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693764-136698891, 136699007- |
| | | | | 136704807, 136704920-136709529, 136709649-136710490, 136710656-136732756, 136732836- |
| | | | | 136742837, 136742938-136787897, |
| 43 | MAP7 | NM_001198614 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693764-136698891, 136699007- |
| | | | | 136704807, 136704920-136709529, 136709649-136710490, 136710656-136732756, 136732836- |
| | | | | 136742837, 136742938-136846968, 136847110-136847498, |
| 43 | MAP7 | NM_001198618 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693764-136698891, 136699007- |
| | | | | 136704807, 136704920-136709529, 136709649-136710490, 136710656-136732756, 136732836- |
| | | | | 136742837, 136742938-136847498, |
| 43 | MAP7 | NM_001198608 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693764-136698891, 136699007- |
| | | | | 136704807, 136704920-136709529, 136709649-136710490, 136710656-136732756, 136732836- |
| | | | | 136742837, 136742938-136846968, |
| 43 | MAP7 | NM_001198609 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693788-136698891, 136699007- |
| | | | | 136704807, 136704920-136709529, 136709649-136710490, 136710656-136732756, 136732836- |
| | | | | 136742837, 136742938-136846968, |
| 43 | MAP7 | NM_001198619 | chr6 | 136665377-136666992, 136667218-136677828, 136677933-136680956, 136681016- |
| | | | | 136681117, 136681170-136681833, 136681937-136682141, 136682318-136683586, 136683841- |
| | | | | 136686871, 136687157-136687463, 136687578-136693637, 136693764-136698891, 136699007- |
| | | | | 136704807, 136704920-136709529, 136709649-136710490, 136710656-136732756, 136732836- |
| | | | | 136742837, 136742938-136847729, |
| 43 | PERP | NM_022121 | chr6 | 138413406-138417489, 138417632-138428262, |
| 43 | HEBP2 | NM_014320 | chr6 | 138725735-138726280, 138726418-138727106, 138727289-138734015, |
| 43 | NHSL1 | NM_020464 | chr6 | 138745954-138750846, 138750981-138751528, 138754818-138768136, 138768331- |
| | | | | 138794441, 138794571-138817354, 138817509-138892845, |
| 43 | NHSL1 | NM_001144060 | chr6 | 138745954-138750846, 138750981-138751528, 138754818-138763118, 138763252- |
| | | | | 138768136, 138768331-138794441, 138794571-138817354, 138817509-138820368, |
| 43 | REPS1 | NM_001128617 | chr6 | 139226270-139228630, 139228738-139229803, 139229954-139232411, 139232509- |
| | | | | 139233900, 139234088-139235832, 139235899-139236993, 139237114-139238690, 139238765- |
| | | | | 139241350, 139241454-139242172, 139242262-139251112, 139251236-139262470, 139262627- |
| | | | | 139264648, 139264714-139264988, 139265153-139265633, 139265760-139266354, 139266510- |
| | | | | 139266636, 139266835-139268925, 139269051-139308665, |
| 43 | REPS1 | NM_031922 | chr6 | 139226270-139228630, 139228738-139229803, 139229954-139232411, 139232509- |
| | | | | 139233900, 139234088-139235832, 139235899-139236993, 139237114-139238690, 139238762- |
| | | | | 139241350, 139241454-139242172, 139242262-139247536, 139247619-139251112, 139251236- |
| | | | | 139262470, 139262627-139264648, 139264714-139264988, 139265153-139265633, 139265760- |
| | | | | 139266354, 139266510-139266636, 139266835-139268925, 139269051-139308665, |
| 43 | GPR126 | NM_198569 | chr6 | 142623469-142630679, 142630782-142688704, 142689048-142691305, 142691931- |
| | | | | 142703086, 142703157-142704895, 142704981-142711393, 142711481-142714083, 142714138- |
| | | | | 142715031, 142715096-142718748, 142718893-142721620, 142721734-142723129, 142723196- |
| | | | | 142723755, 142723968-142724937, 142725111-142726823, 142726966-142729285, 142729407- |
| | | | | 142731009, 142731057-142732440, 142732549-142736104, 142736234-142736931, 142737202- |
| | | | | 142738408, 142738507-142740956, 142741242-142758569, 142758673-142759347, 142759502- |
| | | | | 142764472, |
| 43 | GPR126 | NM_020455 | chr6 | 142623469-142630679, 142630782-142688704, 142689048-142691305, 142691931- |
| | | | | 142703086, 142703157-142704895, 142704981-142711393, 142711481-142714083, 142714138- |
| | | | | 142715031, 142715096-142718748, 142718893-142721620, 142721734-142723129, 142723196- |
| | | | | 142723755, 142723968-142724937, 142725111-142726823, 142726966-142729285, 142729407- |
| | | | | 142731009, 142731057-142732440, 142732549-142736104, 142736234-142736931, 142737202- |
| | | | | 142738408, 142738507-142740956, 142741242-142758569, 142758673-142759347, 142759502- |
| | | | | 142762043, 142762091-142764472, |
| 43 | GPR126 | NM_001032395 | chr6 | 142623469-142630679, 142630782-142688704, 142689048-142691305, 142691931- |
| | | | | 142703086, 142703157-142711393, 142711481-142714083, 142714138-142715031, 142715096- |
| | | | | 142718748, 142718893-142721620, 142721734-142723129, 142723196-142723755, 142723968- |
| | | | | 142724937, 142725111-142726823, 142726966-142729285, 142729407-142731009, 142731057- |
| | | | | 142732440, 142732549-142736104, 142736234-142736931, 142737202-142738408, 142738507- |
| | | | | 142740956, 142741242-142758569, 142758673-142759347, 142759502-142764472, |
| 43 | GPR126 | NM_001032394 | chr6 | 142623469-142630679, 142630782-142688704, 142689048-142691305, 142691931- |
| | | | | 142703086, 142703157-142711393, 142711481-142714083, 142714138-142715031, 142715096- |
| | | | | 142718748, 142718893-142721620, 142721734-142723129, 142723196-142723755, 142723968- |
| | | | | 142724937, 142725111-142726823, 142726966-142729285, 142729407-142731009, 142731057- |
| | | | | 142732440, 142732549-142736104, 142736234-142736931, 142737202-142738408, 142738507- |
| | | | | 142740956, 142741242-142758569, 142758673-142759347, 142759502-142762043, 142762091- |
| | | | | 142764472, |
| 43 | HIVEP2 | NM_006734 | chr6 | 143075069-143080907, 143081805-143082599, 143082703-143085934, 143086112- |
| | | | | 143089517, 143089674-143090687, 143096263-143097282, 143097329-143104656, 143104753- |
| | | | | 143158070, 143158185-143266234, |
| 43 | PHACTR2 | NM_001100166 | chr6 | 143929460-144033151, 144033321-144070120, 144070203-144074889, 144075050- |
| | | | | 144086396, 144086936-144093393, 144093595-144095194, 144095353-144098463, 144098521- |
| | | | | 144104354, 144104418-144109908, 144110048-144128192, 144128271-144144809, |
| 43 | PHACTR2 | NM_014721 | chr6 | 143929460-144033151, 144033321-144070120, 144070203-144074889, 144075050- |
| | | | | 144081536, 144081778-144086396, 144086936-144093393, 144093595-144095194, 144095353- |
| | | | | 144098463, 144098521-144104354, 144104418-144109908, 144110048-144128192, 144128271- |
| | | | | 144144809, |
| 43 | PHACTR2 | NM_001100165 | chr6 | 143999347-144033151, 144033321-144070120, 144070203-144074889, 144075050- |
| | | | | 144086396, 144086936-144093393, 144093595-144095194, 144095353-144098463, 144098521- |
| | | | | 144104354, 144104418-144109908, 144110048-144128192, 144128271-144144809, |
| 43 | PHACTR2 | NM_001100164 | chr6 | 143999347-144033151, 144033321-144070120, 144070203-144074889, 144075050- |
| | | | | 144081536, 144081778-144086396, 144086936-144093393, 144093595-144095194, 144095353- |
| | | | | 144098463, 144098521-144104354, 144104418-144109908, 144110048-144128192, 144128271- |
| | | | | 144144809, |
| 44 | IVNS1ABP | NM_006469 | chr1 | 185267421-185268840, 185269016-185269129, 185269263-185269347, 185269476- |
| | | | | 185269567, 185269691-185270102, 185270329-185270564, 185270696-185274666, 185274776- |
| | | | | 185276144, 185276272-185276619, 185276795-185277930, 185278008-185278133, 185278305- |
| | | | | 185278513, 185278644-185280225, 185280455-185286083, |
| 44 | FAM5C | NM_199051 | chr1 | 190068265-190129796, 190130021-190195210, 190195449-190203500, 190203608- |
| | | | | 190233993, 190234186-190250688, 190250881-190423783, 190424071-190446576, |
| 44 | UCHL5 | NM_015984 | chr1 | 192985526-192990225, 192990326-192992054, 192992170-192992972, 192993077- |
| | | | | 192997213, 192997279-192998307, 192998440-192998516, 192998580-192998660, 192998788- |
| | | | | 193018874, 193018982-193020882, 193020948-193028313, |
| 44 | UCHL5 | NM_001199261 | chr1 | 192985526-192990225, 192990326-192992054, 192992167-192992972, 192993077- |
| | | | | 192997213, 192997279-192998307, 192998440-192998516, 192998580-192998660, 192998788- |
| | | | | 193018874, 193018982-193020882, 193020948-193028313, |
| 44 | UCHL5 | NM_001199262 | chr1 | 192987951-192989435, 192989512-192990225, 192990326-192992054, 192992167- |
| | | | | 192992972, 192993077-192997213, 192997279-192998307, 192998440-192998516, 192998580- |
| | | | | 192998660, 192998788-193018874, 193018982-193020882, 193020948-193028313, |
| 44 | UCHL5 | NM_001199263 | chr1 | 192987951-192990225, 192990326-192992054, 192992167-192992972, 192993077- |
| | | | | 192997213, 192997279-192998307, 192998440-192998516, 192998580-192998660, 192998788- |
| | | | | 193018874, 193018982-193020882, 193020948-193028313, |
| 44 | CDC73 | NM_024529 | chr1 | 193091462-193094240, 193094348-193099302, 193099374-193104519, 193104584- |
| | | | | 193104665, 193104720-193107213, 193107304-193110978, 193111197-193116995, 193117096- |
| | | | | 193119432, 193119513-193121508, 193121575-193172923, 193172983-193181193, 193181231- |
| | | | | 193181518, 193181608-193202121, 193202285-193205384, 193205487-193218858, 193219002- |
| | | | | 193219804, |
| 44 | CFHR1 | NM_002113 | chr1 | 196789033-196794605, 196794802-196795957, 196796136-196797198, 196797377- |
| | | | | 196799628, 196799813-196800925, |
| 44 | CFHR5 | NM_030787 | chr1 | 196946853-196952013, 196952210-196953089, 196953268-196963208, 196963387- |
| | | | | 196964845, 196965030-196965150, 196965332-196967256, 196967435-196971610, 196971795- |
| | | | | 196973789, 196973974-196977615, |
| 44 | ASPM | NM_018136 | chr1 | 197053557-197055931, 197056103-197057384, 197057563-197059058, 197059215- |
| | | | | 197059324, 197059519-197059978, 197060172-197061035, 197061187-197062180, 197062392- |
| | | | | 197063212, 197063311-197065126, 197065295-197069559, 197074316-197086917, 197087114- |
| | | | | 197091043, 197091174-197091287, 197091432-197091516, 197091726-197093238, 197093462- |
| | | | | 197093998, 197094086-197094174, 197094322-197097618, 197097796-197098315, 197098448- |
| | | | | 197099043, 197099187-197101413, 197101483-197102478, 197102726-197104224, 197104373- |
| | | | | 197108895, 197109002-197111459, 197112941-197113085, 197113231-197115269, |
| 44 | KIF14 | NM_014875 | chr1 | 200522896-200523594, 200523735-200524506, 200524583-200528456, 200528581- |
| | | | | 200529848, 200530009-200534195, 200534382-200534571, 200534798-200539037, 200539134- |
| | | | | 200544717, 200544820-200549380, 200549512-200550327, 200550423-200555227, 200555356- |
| | | | | 200558343, 200558498-200559251, 200559401-200561206, 200561369-200562793, 200562898- |
| | | | | 200567363, 200567550-200569176, 200569242-200569482, 200569632-200571022, 200571197- |
| | | | | 200572352, 200572470-200572965, 200573084-200574409, 200574519-200574990, 200575023- |
| | | | | 200575872, 200575927-200577956, 200578057-200583444, 200583534-200584481, 200584738- |
| | | | | 200586738, 200587967-200589537, |
| 44 | C1orf106 | NM_018265 | chr1 | 200860830-200867433, 200867584-200868599, 200868726-200869230, 200869349- |
| | | | | 200870063, 200870288-200876960, 200877016-200877856, 200878069-200878350, 200878526- |
| | | | | 200880579, 200881266-200882663, |
| 44 | C1orf106 | NM_001142569 | chr1 | 200864216-200867433, 200867584-200868599, 200868726-200869230, 200869349- |
| | | | | 200870063, 200870288-200876960, 200877016-200877856, 200878069-200878350, 200878526- |
| | | | | 200880579, 200881266-200882663, |
| 45 | CPLX2 | NM_006650 | chr5 | 175223788-175235990, 175236072-175305653, 175305774-175305909, 175306087-175306849, |
| 45 | CPLX2 | NM_001008220 | chr5 | 175298709-175305653, 175305774-175305909, 175306087-175306849, |
| 45 | FAF2 | NM_014613 | chr5 | 175875472-175906187, 175906258-175913354, 175913491-175915950, 175916029- |
| | | | | 175919193, 175919334-175920998, 175921086-175921183, 175921277-175923485, 175923665- |
| | | | | 175925913, 175926087-175927002, 175927148-175933767, |
| 45 | CDHR2 | NM_017675 | chr5 | 175976416-175992337, 175992406-175992671, 175992745-175995677, 175995819- |
| | | | | 175995959, 175996012-175998212, 175998304-176001082, 176001174-176002083, 176002211- |
| | | | | 176002279, 176002428-176002505, 176002583-176002723, 176002841-176002951, 176003187- |
| | | | | 176004398, 176004556-176004636, 176004800-176005033, 176005097-176005364, 176005610- |
| | | | | 176008342, 176008584-176011189, 176011261-176011409, 176012017-176012953, 176013027- |
| | | | | 176013792, 176013844-176016030, 176016184-176016329, 176016440-176016527, 176016618- |
| | | | | 176017077, 176017148-176017252, 176017332-176017424, 176017487-176017562, 176017689- |
| | | | | 176018209, 176018325-176018403, 176018486-176019722, 176019782-176022531, |
| 45 | CDHR2 | NM_001171976 | chr5 | 175969771-175992337, 175992406-175992671, 175992745-175995677, 175995819- |
| | | | | 175995959, 175996012-175998212, 175998304-176001082, 176001174-176002083, 176002211- |
| | | | | 176002279, 176002428-176002505, 176002583-176002723, 176002841-176002951, 176003187- |
| | | | | 176004398, 176004556-176004636, 176004800-176005033, 176005097-176005364, 176005610- |
| | | | | 176008342, 176008584-176011189, 176011261-176011409, 176012017-176012953, 176013027- |
| | | | | 176013792, 176013844-176016030, 176016184-176016329, 176016440-176016527, 176016618- |
| | | | | 176017077, 176017148-176017252, 176017332-176017424, 176017487-176017562, 176017689- |
| | | | | 176018209, 176018325-176018403, 176018486-176019722, 176019782-176022531, |
| 45 | GPRIN1 | NM_052899 | chr5 | 176026879-176036998, |
| 45 | UIMC1 | NM_016290 | chr5 | 176332494-176333010, 176333049-176334113, 176334179-176335522, 176335696- |
| | | | | 176338310, 176338391-176370334, 176370490-176378473, 176378579-176382958, 176383037- |
| | | | | 176385092, 176385156-176395554, 176396289-176396596, 176396708-176397741, 176397868- |
| | | | | 176402395, 176402482-176409468, 176409625-176433283, |
| 45 | UIMC1 | NM_001199298 | chr5 | 176332494-176333010, 176333049-176334113, 176334179-176335522, 176335696- |
| | | | | 176338310, 176338391-176370334, 176370490-176378473, 176378579-176382958, 176383037- |
| | | | | 176385092, 176385156-176395554, 176396289-176396596, 176396708-176397741, 176397868- |
| | | | | 176402395, 176402482-176409468, 176409625-176433649, |
| 45 | UIMC1 | NM_001199297 | chr5 | 176332494-176333010, 176333049-176334113, 176334179-176335522, 176335696- |
| | | | | 176338310, 176338391-176370334, 176370490-176378473, 176378579-176382958, 176383037- |
| | | | | 176385092, 176385156-176395554, 176396289-176396596, 176396708-176397741, 176397868- |
| | | | | 176402395, 176402482-176409468, 176409625-176428997, 176429113-176433538, |
| 45 | ZNF346 | NM_012279 | chr5 | 176449915-176468125, 176468231-176468777, 176468872-176471388, 176471535- |
| | | | | 176477750, 176477938-176489057, 176489153-176491511, |
| 45 | NSD1 | NM_022455 | chr5 | 176560954-176562086, 176563032-176618883, 176619021-176631119, 176631294- |
| | | | | 176636635, 176639197-176662820, 176662947-176665236, 176665509-176666755, 176666867- |
| | | | | 176671194, 176671272-176673677, 176673798-176675180, 176675326-176678729, 176678855- |
| | | | | 176683950, 176684153-176686988, 176687170-176694561, 176694720-176696601, 176696809- |
| | | | | 176700671, 176700786-176707564, 176707836-176709464, 176709583-176710786, 176710930- |
| | | | | 176715818, 176715927-176718953, 176719160-176720831, |
| 45 | NSD1 | NM_172349 | chr5 | 176560156-176562086, 176562202-176562940, 176563032-176618883, 176619021- |
| | | | | 176631119, 176631294-176636635, 176639197-176662820, 176662947-176665236, 176665509- |
| | | | | 176666755, 176666867-176671194, 176671272-176673677, 176673798-176675180, 176675326- |
| | | | | 176678729, 176678855-176683950, 176684153-176686988, 176687170-176694561, 176694720- |
| | | | | 176696601, 176696809-176700671, 176700786-176707564, 176707836-176709464, 176709583- |
| | | | | 176710786, 176710930-176715818, 176715927-176718953, 176719160-176720831, |
| 46 | MICAL3 | NM_001122731 | chr22 | 18317316-18324586, 18324784-18347663, 18347753-18348688, 18348779-18354601, 18354790- |
| | | | | 18368642, 18368818-18369934, 18369999-18370087, 18370202-18371798, 18371997-18374249, 18374399- |
| | | | | 18376572, 18376671-18378048, 18378177-18379010, 18379128-18379488, 18379748-18382212, 18382315- |
| | | | | 18383606, 18383764-18384642, 18384746-18385395, 18385514-18387396, 18387606-18389313, 18389653- |
| | | | | 18507045, |
| 46 | MICAL3 | NM_001136004 | chr22 | 18347753-18348688, 18348779-18354601, 18354790-18355511, 18355621-18358211, 18358321- |
| | | | | 18362110, 18362169-18363969, 18364070-18368642, 18368818-18369934, 18369999-18370087, 18370202- |
| | | | | 18371798, 18371997-18374249, 18374399-18376572, 18376671-18378048, 18378177-18379010, 18379128- |
| | | | | 18379488, 18379748-18382212, 18382315-18383606, 18383764-18384642, 18384746-18385395, 18385514- |
| | | | | 18387396, 18387606-18389313, 18389653-18507045, |
| 46 | MICAL3 | NM_015241 | chr22 | 18273683-18273762, 18273838-18273966, 18274068-18291608, 18291704-18293467, 18293580- |
| | | | | 18299454, 18299551-18300075, 18301896-18304191, 18304276-18304794, 18304937-18305711, 18305827- |
| | | | | 18310408, 18310548-18314618, 18314874-18324586, 18324784-18347663, 18347753-18348688, 18348779- |
| | | | | 18354601, 18354790-18368642, 18368818-18369934, 18369999-18370087, 18370202-18371798, 18371997- |
| | | | | 18374249, 18374399-18376572, 18376671-18378048, 18378177-18379010, 18379128-18379488, 18379748- |
| | | | | 18382212, 18382315-18383606, 18383764-18384642, 18384746-18385395, 18385514-18387396, 18387606- |
| | | | | 18389313, 18389653-18507045, |
| 46 | UFD1L | NM_001035247 | chr22 | 19438268-19442323, 19442354-19443201, 19443292-19444108, 19444158-19444374, 19444442- |
| | | | | 19445592, 19445663-19452723, 19452798-19455394, 19455527-19459208, 19459332-19462589, 19462624- |
| | | | | 19462991, 19463126-19466604, |
| 46 | UFD1L | NM_005659 | chr22 | 19438268-19442270, 19442354-19443201, 19443292-19444108, 19444158-19444374, 19444442- |
| | | | | 19445592, 19445663-19452723, 19452798-19455394, 19455527-19459208, 19459332-19462589, 19462624- |
| | | | | 19462991, 19463126-19466604, |
| 46 | CDC45 | NM_001178011 | chr22 | 19467543-19467679, 19467741-19468474, 19468569-19471383, 19471529-19481848, 19481906- |
| | | | | 19483502, 19483553-19484907, 19484971-19486622, 19486675-19492883, 19493005-19494907, 19495041- |
| | | | | 19495287, 19495388-19496051, 19496215-19502270, 19502411-19502486, 19502572-19504048, 19504169- |
| | | | | 19504338, 19504417-19506365, 19506433-19508002, |
| 46 | CDC45 | NM_001178010 | chr22 | 19467543-19467679, 19467741-19468474, 19468569-19470211, 19470351-19471383, 19471529- |
| | | | | 19481848, 19481906-19482028, 19482126-19483502, 19483553-19484907, 19484971-19486622, 19486675- |
| | | | | 19492883, 19493005-19494907, 19495041-19495287, 19495388-19496051, 19496215-19502270, 19502411- |
| | | | | 19502486, 19502572-19504048, 19504169-19504338, 19504417-19506365, 19506433-19508002, |
| 46 | CDC45 | NM_003504 | chr22 | 19467543-19467679, 19467741-19468474, 19468569-19470211, 19470351-19471383, 19471529- |
| | | | | 19481848, 19481906-19483502, 19483553-19484907, 19484971-19486622, 19486675-19492883, 19493005- |
| | | | | 19494907, 19495041-19495287, 19495388-19496051, 19496215-19502270, 19502411-19502486, 19502572- |
| | | | | 19504048, 19504169-19504338, 19504417-19506365, 19506433-19508002, |
| 46 | PRAME | NM_206954 | chr22 | 22891066-22892146, 22892757-22893187, 22893512-22899230, 22899330-22899963, 22900001-22901580, |
| 46 | PRAME | NM_206953 | chr22 | 22891066-22892146, 22892757-22893187, 22893512-22899230, 22900001-22901580, |
| 46 | PRAME | NM_006115 | chr22 | 22891066-22892146, 22892757-22893187, 22893512-22899230, 22899351-22899963, 22900001-22901580, |
| 46 | PRAME | NM_206956 | chr22 | 22891066-22892146, 22892757-22893187, 22893512-22899230, 22899330-22899963, 22900001-22901376, |
| 46 | PRAME | NM_206955 | chr22 | 22891066-22892146, 22892757-22893187, 22893512-22899230, 22899351-22899963, 22900001-22901376, |
| 46 | LOC91316 | NR_024448 | chr22 | 23981130-24002050, 24002188-24025910, 24026055-24032421, 24032544-24036304, 24037705- |
| | | | | 24042911, 24043033-24047614, 24047729-24051315, 24051429-24051971, 24052055-24056372, 24056530- |
| | | | | 24057194, 24057382-24059216, |
| 46 | CHCHD10 | NM_213720 | chr22 | 24108214-24108313, 24108463-24109559, 24109781-24110019, |
| 47 | PRR11 | NM_018304 | chr17 | 57233167-57247107, 57247242-57262413, 57262566-57262799, 57262924-57270851, 57271096- |
| | | | | 57272116, 57272217-57272690, 57272805-57274903, 57274965-57275052, 57275151-57278922, |
| 47 | DHX40 | NM_001166301 | chr17 | 57643145-57643986, 57644071-57650475, 57650597-57651099, 57651329-57651547, 57651616- |
| | | | | 57652699, 57652833-57654625, 57654727-57656831, 57656920-57657056, 57657241-57663512, 57663595- |
| | | | | 57665255, 57665415-57676096, 57676213-57676734, 57676845-57679881, 57679978-57682474, 57682546- |
| | | | | 57682805, 57683036-57684392, |
| 47 | DHX40 | NM_024612 | chr17 | 57643145-57643986, 57644156-57647877, 57648025-57650475, 57650597-57651099, 57651329- |
| | | | | 57651547, 57651616-57652699, 57652833-57654625, 57654727-57656831, 57656920-57657056, 57657241- |
| | | | | 57663512, 57663595-57665255, 57665415-57676096, 57676213-57676734, 57676845-57679881, 57679978- |
| | | | | 57682474, 57682546-57682805, 57683036-57684392, |
| 47 | CLTC | NM_004859 | chr17 | 57697535-57721635, 57721845-57724757, 57725028-57725599, 57725763-57728562, 57728678- |
| | | | | 57733213, 57733389-57737750, 57737950-57738802, 57739005-57741201, 57741356-57742146, 57742271- |
| | | | | 57743462, 57743602-57743839, 57744006-57744156, 57744339-57746136, 57746302-57751006, 57751134- |
| | | | | 57752060, 57752205-57754313, 57754550-57756756, 57756881-57758271, 57758419-57758654, 57758840- |
| | | | | 57759006, 57759201-57759630, 57759790-57759988, 57760155-57760266, 57760376-57760454, 57760624- |
| | | | | 57760746, 57760898-57760984, 57761118-57761234, 57761347-57762415, 57762588-57762946, 57763170- |
| | | | | 57767995, 57768073-57771087, |
| 47 | PTRH2 | NM_016077 | chr17 | 57775340-57784730, |
| 47 | TMEM49 | NM_030938 | chr17 | 57785110-57808780, 57808884-57812697, 57812835-57814812, 57814905-57816196, 57816309- |
| | | | | 57842330, 57842500-57851113, 57851247-57886155, 57886238-57889029, 57889148-57895071, 57895135- |
| | | | | 57915654, 57915759-57917127, |
| 47 | HEATR6 | NM_022070 | chr17 | 58121496-58123384, 58123591-58124668, 58124767-58125623, 58125750-58126939, 58127064- |
| | | | | 58128202, 58128339-58132874, 58132963-58133474, 58133599-58134407, 58134765-58136781, 58136883- |
| | | | | 58137249, 58137458-58143568, 58143748-58144793, 58145094-58147070, 58147210-58148065, 58148169- |
| | | | | 58149574, 58149691-58150523, 58150641-58151105, 58151248-58153489, 58153599-58156055, |
| 47 | C17orf64 | NM_181707 | chr17 | 58500005-58503142, 58503246-58503520, 58503698-58504077, 58504179-58506721, 58506919-58508540, |
| 47 | BCAS3 | NM_001099432 | chr17 | 58755276-58756812, 58756902-58761341, 58761398-58767045, 58767123-58786578, 58786687- |
| | | | | 58824557, 58824641-58885363, 58885438-58945935, 58946045-58952021, 58952100-58967054, 58967133- |
| | | | | 58979979, 58980065-58987891, 58988064-59001766, 59001862-59024578, 59024714-59067330, 59067597- |
| | | | | 59093110, 59093263-59104225, 59104272-59112025, 59112152-59115248, 59115416-59118151, 59118254- |
| | | | | 59152279, 59152382-59155692, 59155891-59161826, 59161926-59445686, 59445856-59469336, |
| 47 | BCAS3 | NM_017679 | chr17 | 58755276-58756812, 58756902-58761341, 58761398-58767045, 58767123-58786578, 58786687- |
| | | | | 58824557, 58824641-58885363, 58885438-58945935, 58946045-58952021, 58952100-58967054, 58967133- |
| | | | | 58979979, 58980065-58987891, 58988064-59001766, 59001862-59024578, 59024714-59067330, 59067597- |
| | | | | 59093110, 59093263-59112025, 59112152-59115248, 59115416-59118151, 59118254-59152279, 59152382- |
| | | | | 59155692, 59155891-59161826, 59161926-59445686, 59445856-59469336, |
| 47 | TBX4 | NM_018488 | chr17 | 59534038-59534896, 59534993-59543178, 59543300-59544869, 59545019-59555986, 59556141- |
| | | | | 59557240, 59557331-59557449, 59557681-59560259, |
| 47 | BRIP1 | NM_032043 | chr17 | 59761502-59763195, 59763527-59770789, 59770874-59793310, 59793425-59820372, 59820496- |
| | | | | 59821791, 59821953-59853760, 59853924-59857620, 59857763-59858199, 59858367-59861629, 59861786- |
| | | | | 59870956, 59871091-59876459, 59876661-59878612, 59878836-59885826, 59886119-59924460, 59924582- |
| | | | | 59926488, 59926618-59934417, 59934593-59937155, 59937269-59938806, 59938931-59940643, |
| 47 | MED13 | NM_005121 | chr17 | 60023962-60024276, 60024379-60028184, 60028360-60030324, 60030475-60032741, 60032929- |
| | | | | 60033039, 60033200-60033698, 60033843-60038226, 60038452-60038947, 60039141-60040111, 60040333- |
| | | | | 60042365, 60042830-60043821, 60044013-60045394, 60045620-60050086, 60050250-60059557, 60060476- |
| | | | | 60061530, 60061729-60062010, 60062227-60062359, 60062452-60064366, 60064490-60069899, 60069983- |
| | | | | 60072511, 60072727-60087909, 60088595-60106900, 60107013-60107210, 60107375-60108803, 60109000- |
| | | | | 60111146, 60111346-60112822, 60112970-60129896, 60130067-60140426, 60140663-60142499, |
| 47 | STRADA | NM_001003786 | chr17 | 61781112-61781363, 61781408-61781699, 61781943-61783993, 61784100-61784605, 61784779- |
| | | | | 61787849, 61787975-61788086, 61788197-61790764, 61790888-61791364, 61791469-61805716, 61805774- |
| | | | | 61819099, |
| 47 | STRADA | NM_001003787 | chr17 | 61781112-61781363, 61781408-61781699, 61781943-61783993, 61784100-61784605, 61784779- |
| | | | | 61787849, 61787975-61788086, 61788197-61790764, 61790888-61791364, 61791469-61800656, 61800687- |
| | | | | 61803996, 61804056-61805692, 61805774-61819099, |
| 47 | STRADA | NM_001165969 | chr17 | 61781112-61781363, 61781408-61781790, 61781943-61783993, 61784100-61784605, 61784779- |
| | | | | 61787849, 61787975-61788086, 61788197-61790764, 61790888-61791364, 61791469-61805692, 61805774- |
| | | | | 61819099, |
| 47 | STRADA | NM_153335 | chr17 | 61781943-61783993, 61784100-61784605, 61784779-61787849, 61787975-61788086, 61788197- |
| | | | | 61790764, 61790888-61791364, 61791469-61805716, 61805774-61819099, |
| 47 | STRADA | NM_001165970 | chr17 | 61781112-61781363, 61781408-61781790, 61781943-61783993, 61784100-61784605, 61784779- |
| | | | | 61787849, 61787975-61788086, 61788197-61790764, 61790888-61803996, 61804056-61805692, 61805774- |
| | | | | 61819099, |
| 47 | STRADA | NM_001003788 | chr17 | 61781112-61781363, 61781408-61781699, 61781943-61783993, 61784100-61784605, 61784779- |
| | | | | 61787849, 61787975-61788086, 61788197-61790764, 61790888-61791364, 61791469-61803996, 61804056- |
| | | | | 61805692, 61805774-61819099, |
| 47 | CCDC47 | NM_020198 | chr17 | 61824322-61829298, 61829468-61829678, 61829790-61830099, 61830160-61831791, 61831879- |
| | | | | 61833598, 61833711-61833827, 61833931-61838272, 61838340-61838588, 61838712-61841374, 61841551- |
| | | | | 61842098, 61842208-61843270, 61843555-61850770, |
| 47 | DDX42 | NM_007372 | chr17 | 61851737-61852466, 61852539-61864392, 61864631-61869770, 61869923-61875396, 61875460- |
| | | | | 61876947, 61876986-61877826, 61877978-61882429, 61882536-61883893, 61884015-61885082, 61885261- |
| | | | | 61886178, 61886309-61886918, 61887020-61887871, 61887921-61888434, 61888534-61889290, 61889569- |
| | | | | 61890586, 61890815-61892921, 61893034-61894226, 61894327-61895052, |
| 47 | DDX42 | NM_203499 | chr17 | 61851737-61864392, 61864631-61869770, 61869923-61875396, 61875460-61876947, 61876986- |
| | | | | 61877826, 61877978-61882429, 61882536-61883893, 61884015-61885082, 61885261-61886178, 61886309- |
| | | | | 61886918, 61887020-61887871, 61887921-61888434, 61888534-61889290, 61889569-61890586, 61890815- |
| | | | | 61892921, 61893034-61894226, 61894327-61895052, |
| 47 | FTSJ3 | NM_017647 | chr17 | 61897355-61897437, 61897534-61897613, 61897799-61898197, 61898299-61898388, 61898476- |
| | | | | 61898712, 61899004-61899081, 61899204-61899342, 61899529-61901175, 61901313-61901442, 61901600- |
| | | | | 61901689, 61901772-61901995, 61902098-61902183, 61902290-61902406, 61902524-61902600, 61902797- |
| | | | | 61902894, 61902996-61903414, 61903496-61903616, 61903665-61903925, 61904033-61904194, 61904289- |
| | | | | 61904410, |
| 47 | SMARCD2 | NM_001098426 | chr17 | 61910352-61910427, 61910531-61910656, 61910781-61910945, 61911083-61911267, 61911367- |
| | | | | 61911525, 61911689-61911832, 61911936-61912063, 61912161-61912770, 61912928-61914280, 61914405- |
| | | | | 61914553, 61914598-61914799, 61914986-61919817, |
| 47 | TCAM1P | NR_002947 | chr17 | 61934784-61936662, 61936985-61937232, 61937657-61938046, 61938315-61939647, 61939892- |
| | | | | 61940083, 61940491-61940515, |
| 47 | CSHL1 | NM_001318 | chr17 | 61987269-61987520, 61987687-61987778, 61987900-61988545, |
| 47 | CSHL1 | NM_022579 | chr17 | 61987269-61987520, 61987687-61987778, 61987896-61988103, 61988285-61988545, |
| 47 | CSHL1 | NM_022581 | chr17 | 61987269-61987520, 61987687-61987778, 61987827-61988103, 61988285-61988545, |
| 47 | CSHL1 | NM_022580 | chr17 | 61987269-61987520, 61987687-61987778, 61987827-61988545, |
| 47 | TEX2 | NM_018469 | chr17 | 62226452-62228199, 62228322-62230303, 62230515-62232200, 62232328-62238159, 62238294- |
| | | | | 62248458, 62248560-62254683, 62254832-62265526, 62265797-62270917, 62271250-62272253, 62272456- |
| | | | | 62289932, 62291603-62340526, |
| 47 | POLG2 | NM_007215 | chr17 | 62474106-62476404, 62476507-62479034, 62479117-62481843, 62481986-62486911, 62487087- |
| | | | | 62488782, 62488890-62489010, 62489139-62492523, |
| 47 | DDX5 | NM_004396 | chr17 | 62496445-62496665, 62496892-62498126, 62498188-62498278, 62498342-62498555, 62498668- |
| | | | | 62499042, 62499217-62499304, 62499467-62499546, 62499690-62499919, 62499987-62500099, 62500235- |
| | | | | 62500338, 62500437-62500793, 62500961-62502192, |
| 47 | CCDC45 | NM_138363 | chr17 | 62503235-62504708, 62504839-62506289, 62506399-62510364, 62510477-62512839, 62512947- |
| | | | | 62515437, 62515555-62517518, 62517646-62518818, 62519014-62521886, 62522001-62522187, 62522319- |
| | | | | 62523227, 62523383-62525404, 62525546-62527042, 62527137-62528006, 62528141-62528955, 62529127- |
| | | | | 62529233, 62529310-62530701, 62530856-62532718, 62532867-62533157, 62533231-62533719, |
| 47 | SMURF2 | NM_022739 | chr17 | 62542066-62542379, 62542457-62543716, 62543920-62547700, 62547823-62550972, 62551112- |
| | | | | 62551936, 62552117-62553724, 62553841-62557616, 62557722-62558887, 62559085-62567914, 62568075- |
| | | | | 62574608, 62574695-62576905, 62577110-62579577, 62579663-62582202, 62582289-62587200, 62587268- |
| | | | | 62589556, 62589692-62594498, 62594609-62602718, 62602759-62657945, |
| 47 | LOC146880 | NR_027487 | chr17 | 62776324-62776459, 62777158-62777511, |
| 47 | LOC146880 | NR_026899 | chr17 | 62746127-62746710, 62746842-62747923, 62748075-62750121, 62750283-62750537, 62750773- |
| | | | | 62754358, 62754498-62754645, 62754745-62758032, 62758166-62758471, 62758829-62760567, 62760667- |
| | | | | 62776459, 62777158-62777550, 62777799-62778041, |
| 47 | PLEKHM1P | NR_024386 | chr17 | 62781486-62782683, 62782843-62783364, 62783430-62788465, 62788661-62793380, 62793528- |
| | | | | 62796116, 62797036-62800946, 62801219-62817883, 62818171-62818370, 62818512-62825277, 62825368- |
| | | | | 62833172, |
| 47 | NOL11 | NM_015462 | chr17 | 65714205-65715788, 65715904-65716020, 65716079-65717492, 65717643-65718694, 65718754- |
| | | | | 65720163, 65720310-65722574, 65722765-65730477, 65730556-65732014, 65732140-65732780, 65732871- |
| | | | | 65733185, 65733262-65733622, 65733809-65733961, 65734089-65734236, 65734472-65735046, 65735127- |
| | | | | 65735630, 65735725-65739593, 65739703-65739857, |
| 47 | BPTF | NM_182641 | chr17 | 65822454-65850054, 65850879-65862578, 65862804-65870931, 65871137-65887958, 65888151- |
| | | | | 65889484, 65889842-65890148, 65890282-65899903, 65900035-65900816, 65900957-65905696, 65905878- |
| | | | | 65906991, 65909304-65914828, 65914955-65916129, 65916260-65918954, 65919107-65920661, 65920706- |
| | | | | 65924469, 65924718-65925450, 65925604-65928025, 65928136-65936553, 65936773-65940264, 65940489- |
| | | | | 65941523, 65942442-65943840, 65943925-65944195, 65944423-65955655, 65955992-65960326, 65960521- |
| | | | | 65962686, 65962773-65971886, 65972075-65978366, |
| 47 | BPTF | NM_004459 | chr17 | 65822454-65850054, 65850879-65862578, 65862804-65870931, 65871137-65871670, 65871861- |
| | | | | 65882242, 65882433-65887958, 65888151-65889484, 65889842-65890148, 65890282-65899903, 65900035- |
| | | | | 65900816, 65900957-65905696, 65905878-65906991, 65909304-65914828, 65914955-65916129, 65916260- |
| | | | | 65918954, 65919107-65920661, 65920706-65924469, 65924718-65925450, 65925604-65928025, 65928136- |
| | | | | 65936553, 65936773-65940264, 65940489-65941523, 65942013-65943840, 65943925-65944195, 65944423- |
| | | | | 65955655, 65955992-65960326, 65960521-65962686, 65962773-65971886, 65972075-65978366, |
| 47 | KPNA2 | NM_002266 | chr17 | 66031957-66033224, 66033324-66033472, 66033612-66036792, 66036883-66038199, 66038470- |
| | | | | 66039019, 66039116-66039214, 66039480-66039952, 66040188-66040435, 66040620-66041886, 66042038- |
| | | | | 66042618, |
| 47 | FAM20A | NM_017565 | chr17 | 66533883-66535476, 66535538-66536024, 66536108-66536988, 66537100-66538124, 66538307- |
| | | | | 66538833, 66538951-66539768, 66539863-66548012, 66548093-66550916, 66550969-66551698, 66551885- |
| | | | | 66596402, |
| 47 | FAM20A | NR_027751 | chr17 | 66533883-66535476, 66535538-66536024, 66536108-66536988, 66537125-66538124, 66538307- |
| | | | | 66538833, 66538951-66539768, 66539859-66548012, 66548093-66550916, 66550969-66551698, 66551885- |
| | | | | 66554469, |
| 48 | SHCBP1 | NM_024745 | chr16 | 46615967-46617426, 46617570-46617913, 46618002-46629462, 46629583-46633741, 46633875- |
| | | | | 46637449, 46637572-46637885, 46638056-46638138, 46638374-46642207, 46642302-46649856, 46650067- |
| | | | | 46651544, 46651662-46652115, 46652285-46655167, |
| 48 | ORC6 | NM_014321 | chr16 | 46723683-46724901, 46725033-46726292, 46726458-46727003, 46727095-46729472, 46729587- |
| | | | | 46729927, 46729998-46731368, |
| 48 | ITFG1 | NM_030790 | chr16 | 47189690-47192782, 47192902-47195659, 47195744-47196449, 47196576-47252777, 47252858- |
| | | | | 47271874, 47271920-47292541, 47292652-47294454, 47294607-47345149, 47345324-47347638, 47347735- |
| | | | | 47399697, 47399781-47409785, 47409852-47462713, 47462810-47485305, 47485382-47486642, 47486702- |
| | | | | 47487922, 47488070-47493012, 47493087-47494747, |
| 48 | PHKB | NM_000293 | chr16 | 47495338-47531308, 47531400-47533665, 47533806-47536900, 47537002-47545574, 47545684- |
| | | | | 47549430, 47549513-47581342, 47581460-47614204, 47614270-47621577, 47621675-47622814, 47623014- |
| | | | | 47627409, 47627469-47628046, 47628126-47630282, 47630443-47644735, 47644832-47674944, 47675002- |
| | | | | 47675508, 47675604-47682442, 47682528-47683009, 47683116-47684453, 47684538-47684740, 47684833- |
| | | | | 47694415, 47694479-47694566, 47694731-47695628, 47695712-47697586, 47697646-47698794, 47698887- |
| | | | | 47703124, 47703329-47722950, 47723087-47727287, 47727419-47730290, 47730400-47732357, 47732500- |
| | | | | 47733138, |
| 48 | PHKB | NM_001031835 | chr16 | 47495338-47497791, 47497905-47531308, 47531400-47533665, 47533806-47536900, 47537002- |
| | | | | 47545574, 47545684-47549430, 47549513-47581342, 47581460-47614204, 47614270-47621577, 47621675- |
| | | | | 47622814, 47623014-47627409, 47627469-47628046, 47628126-47630282, 47630443-47644735, 47644832- |
| | | | | 47674944, 47675002-47675508, 47675604-47682442, 47682528-47683009, 47683116-47684453, 47684538- |
| | | | | 47684740, 47684833-47694415, 47694479-47694566, 47694731-47695628, 47695712-47697586, 47697646- |
| | | | | 47699851, 47699944-47703124, 47703329-47722950, 47723087-47727287, 47727419-47730290, 47730400- |
| | | | | 47732357, 47732500-47733138, |
| 48 | HEATR3 | NM_182922 | chr16 | 50100181-50100276, 50100451-50102689, 50102779-50104087, 50104202-50106514, 50106626- |
| | | | | 50109480, 50109623-50112650, 50112930-50117846, 50117939-50118043, 50118203-50118478, 50118563- |
| | | | | 50120124, 50120263-50128614, 50128705-50134139, 50134285-50136168, 50136347-50138848, |
| 48 | BRD7 | NM_001173984 | chr16 | 50353178-50353796, 50353942-50354157, 50354303-50354572, 50354689-50355891, 50355950- |
| | | | | 50357496, 50357610-50359657, 50359795-50360193, 50360303-50362578, 50362656-50367483, 50367609- |
| | | | | 50368620, 50368807-50373885, 50373998-50383932, 50384079-50388334, 50388394-50388702, 50388834- |
| | | | | 50401999, 50402210-50402635, |
| 48 | BRD7 | NM_013263 | chr16 | 50353178-50353796, 50353942-50354157, 50354303-50354572, 50354686-50355891, 50355950- |
| | | | | 50357496, 50357610-50359657, 50359795-50360193, 50360303-50362578, 50362656-50367483, 50367609- |
| | | | | 50368620, 50368807-50373885, 50373998-50383932, 50384079-50388334, 50388394-50388702, 50388834- |
| | | | | 50401999, 50402210-50402635, |
| 48 | NUDT21 | NM_007006 | chr16 | 56466646-56468241, 56468358-56468664, 56468742-56473567, 56473659-56480536, 56480602- |
| | | | | 56481699, 56481902-56484997, |
| 48 | MT4 | NM_032935 | chr16 | 56599072-56601661, 56601729-56602751, |
| 48 | MT1G | NM_005950 | chr16 | 56700883-56701222, 56701290-56701876, |
| 48 | NUP93 | NM_014669 | chr16 | 56764124-56782144, 56782339-56792448, 56792568-56832386, 56832451-56839414, 56839545- |
| | | | | 56852574, 56852651-56855414, 56855506-56857617, 56857759-56862887, 56863022-56864438, 56864598- |
| | | | | 56865752, 56865920-56866205, 56866301-56867125, 56867319-56868038, 56868167-56868280, 56868355- |
| | | | | 56868644, 56868691-56870511, 56870630-56871518, 56871639-56872862, 56872982-56873431, 56873517- |
| | | | | 56875615, 56875746-56878409, |
| 48 | HERPUD1 | NM_001010990 | chr16 | 56966304-56969145, 56969225-56970597, 56970730-56973147, 56973272-56973805, 56974158- |
| | | | | 56976042, 56976150-56977036, |
| 48 | HERPUD1 | NM_001010989 | chr16 | 56966304-56969145, 56969225-56969314, 56969388-56970597, 56970730-56973147, 56973272- |
| | | | | 56973805, 56974158-56976042, 56976150-56977036, |
| 48 | HERPUD1 | NM_014685 | chr16 | 56966304-56969145, 56969225-56969311, 56969388-56970597, 56970730-56973147, 56973272- |
| | | | | 56973805, 56974158-56976042, 56976150-56977036, |
| 48 | COQ9 | NM_020312 | chr16 | 57481491-57484950, 57485121-57486711, 57486849-57490414, 57490559-57490841, 57490928- |
| | | | | 57492156, 57492263-57493475, 57493633-57493961, 57494017-57494499, |
| 48 | GPR56 | NM_001145774 | chr16 | 57673583-57675501, 57675621-57684163, 57684264-57685110, 57685535-57687113, 57687248- |
| | | | | 57687896, 57688046-57689309, 57689443-57689786, 57689905-57690136, 57690184-57690424, 57690530- |
| | | | | 57691283, 57691404-57693323, 57693594-57694678, 57694789-57695607, 57695878-57697362, |
| 48 | GPR56 | NM_201524 | chr16 | 57662715-57675497, 57675621-57684163, 57684264-57685110, 57685535-57687113, 57687248- |
| | | | | 57687896, 57688046-57689309, 57689443-57689786, 57689905-57690136, 57690184-57690424, 57690530- |
| | | | | 57691283, 57691404-57693323, 57693594-57694678, 57694789-57695607, 57695878-57697362, |
| 48 | GPR56 | NM_005682 | chr16 | 57654049-57675501, 57675621-57684163, 57684264-57685110, 57685535-57687113, 57687248- |
| | | | | 57687896, 57688046-57689309, 57689443-57689786, 57689905-57690136, 57690184-57690424, 57690530- |
| | | | | 57691283, 57691404-57693305, 57693594-57694678, 57694789-57695607, 57695878-57697362, |
| 48 | GPR56 | NM_001145771 | chr16 | 57662715-57675501, 57675621-57684163, 57684264-57685110, 57685535-57687113, 57687248- |
| | | | | 57687896, 57688046-57689309, 57689443-57689786, 57689905-57690136, 57690184-57690424, 57690530- |
| | | | | 57691283, 57691404-57693305, 57693594-57694678, 57694789-57695607, 57695878-57697362, |
| 48 | GPR56 | NM_001145770 | chr16 | 57654049-57675501, 57675621-57684163, 57684264-57685110, 57685535-57687113, 57687248- |
| | | | | 57687896, 57688046-57689309, 57689443-57689786, 57689905-57690136, 57690184-57690424, 57690530- |
| | | | | 57691283, 57691404-57693323, 57693594-57694678, 57694789-57695607, 57695878-57697362, |
| 48 | GPR56 | NM_001145773 | chr16 | 57662715-57684163, 57684264-57685095, 57685535-57687113, 57687248-57687896, 57688046- |
| | | | | 57689309, 57689443-57689786, 57689905-57690136, 57690184-57690424, 57690530-57691283, 57691404- |
| | | | | 57693323, 57693594-57694678, 57694789-57695607, 57695878-57697362, |
| 48 | GPR56 | NM_001145772 | chr16 | 57662741-57675501, 57675621-57684163, 57684264-57685110, 57685535-57687113, 57687248- |
| | | | | 57687896, 57688046-57689309, 57689443-57689786, 57689905-57690136, 57690184-57690424, 57690530- |
| | | | | 57691283, 57691404-57693323, 57693594-57694678, 57694789-57695607, 57695878-57697362, |
| 48 | GPR56 | NM_201525 | chr16 | 57662715-57684163, 57684264-57685110, 57685535-57687113, 57687248-57687896, 57688046- |
| | | | | 57689309, 57689443-57689786, 57689905-57690136, 57690184-57690424, 57690530-57691283, 57691404- |
| | | | | 57693323, 57693594-57694678, 57694789-57695607, 57695878-57697362, |
| 48 | MMP15 | NM_002428 | chr16 | 58060417-58071374, 58071525-58072168, 58072299-58073777, 58074087-58074439, 58074603- |
| | | | | 58075519, 58075775-58076133, 58076274-58077112, 58077265-58077414, 58077532-58078909, |
| 48 | CNOT1 | NM_016284 | chr16 | 58554941-58555085, 58555222-58557272, 58557407-58559081, 58559264-58559891, 58560043- |
| | | | | 58562377, 58562553-58564148, 58564252-58565860, 58565980-58566134, 58566300-58568049, 58568300- |
| | | | | 58570891, 58571125-58571793, 58571965-58572060, 58572171-58572674, 58572819-58573670, 58573864- |
| | | | | 58575403, 58575525-58576133, 58576240-58576330, 58576473-58577509, 58577808-58579263, 58579396- |
| | | | | 58580223, 58580403-58580574, 58580654-58581088, 58581201-58581468, 58581587-58583621, 58583803- |
| | | | | 58585034, 58585177-58585491, 58585724-58587684, 58587765-58589153, 58589442-58589686, 58589813- |
| | | | | 58590749, 58590898-58592375, 58592579-58594114, 58594267-58608511, 58608665-58608909, 58609034- |
| | | | | 58610365, 58610487-58612601, 58612844-58614535, 58614665-58615247, 58615420-58616695, 58616808- |
| | | | | 58616958, 58617087-58619240, 58619411-58620447, 58620653-58621103, 58621160-58621247, 58621318- |
| | | | | 58621682, 58621783-58622701, 58622811-58633138, 58633416-58663630, |
| 48 | CNOT1 | NM_206999 | chr16 | 58577808-58579263, 58579396-58580223, 58580403-58580574, 58580654-58581088, 58581201- |
| | | | | 58581468, 58581587-58583621, 58583803-58585034, 58585177-58585491, 58585724-58587684, 58587765- |
| | | | | 58589153, 58589442-58589686, 58589813-58590749, 58590898-58592375, 58592579-58594114, 58594267- |
| | | | | 58608511, 58608665-58608909, 58609034-58610365, 58610487-58612601, 58612844-58614535, 58614665- |
| | | | | 58615247, 58615420-58616695, 58616808-58616958, 58617087-58619240, 58619411-58620447, 58620653- |
| | | | | 58621103, 58621160-58621247, 58621318-58621682, 58621783-58622701, 58622811-58633138, 58633416- |
| | | | | 58663630, |
| 48 | CDH8 | NM_001796 | chr16 | 61688006-61689372, 61689626-61747743, 61747863-61760996, 61761120-61823248, 61823387- |
| | | | | 61851381, 61851637-61854828, 61855018-61858914, 61859084-61891021, 61891143-61935081, 61935378- |
| | | | | 62055054, 62055507-62069982, |
| 48 | DYNC1LI2 | NM_006141 | chr16 | 66757726-66759729, 66759848-66761589, 66761709-66762102, 66762146-66762941, 66763003- |
| | | | | 66764013, 66764127-66766240, 66766378-66768119, 66768215-66769976, 66770148-66776339, 66776572- |
| | | | | 66783098, 66783217-66785174, 66785250-66785386, |
| 48 | CBFB | NM_022845 | chr16 | 67063389-67063628, 67063717-67070540, 67070659-67100583, 67100702-67116114, 67116212-67132611, |
| 48 | CBFB | NM_001755 | chr16 | 67063389-67063628, 67063717-67070540, 67070659-67100583, 67100702-67116114, 67116243-67132611, |
| 48 | EXOC3L | NM_178516 | chr16 | 67218508-67218587, 67218714-67218803, 67218961-67219037, 67219162-67219262, 67219394- |
| | | | | 67220138, 67220251-67220458, 67220561-67220660, 67220788-67220922, 67221042-67221126, 67221741- |
| | | | | 67222622, 67222844-67222921, 67223084-67223532, 67223587-67223872, |
| 48 | GFOD2 | NM_030819 | chr16 | 67709957-67719358, 67719706-67753064, |
| 48 | GFOD2 | NR_027398 | chr16 | 67709957-67753064, |
| 48 | GFOD2 | NR_027399 | chr16 | 67719706-67753064, |
| 48 | NUTF2 | NM_005796 | chr16 | 67880889-67899003, 67899133-67902241, 67902315-67902402, 67902503-67904701, |
| 48 | DUS2L | NM_017803 | chr16 | 68057255-68059316, 68059398-68071907, 68072053-68083406, 68083454-68087465, 68087559- |
| | | | | 68088475, 68088521-68090265, 68090328-68094967, 68095017-68100218, 68100286-68100467, 68100540- |
| | | | | 68104035, 68104124-68104841, 68105012-68107935, 68108059-68109256, 68109408-68110533, 68110623- |
| | | | | 68112346, 68112422-68112650, |
| 48 | ESRP2 | NM_024939 | chr16 | 68264219-68264290, 68264458-68264742, 68264931-68265079, 68265280-68265383, 68265598- |
| | | | | 68265703, 68266007-68266147, 68266192-68266272, 68266407-68266478, 68266591-68266662, 68266719- |
| | | | | 68267228, 68267329-68267669, 68267786-68267895, 68268011-68269535, 68269666-68269749, |
| 48 | WWP2 | NM_199424 | chr16 | 69959388-69963349, 69963433-69964031, 69964162-69965053, 69965131-69965410, 69965484- |
| | | | | 69965703, 69965794-69967871, 69968033-69969754, 69969890-69970213, 69970356-69971019, 69971142- |
| | | | | 69971449, 69971556-69972928, 69973027-69973242, 69973317-69973742, |
| 48 | WWP2 | NM_007014 | chr16 | 69796295-69810090, 69810145-69820897, 69820984-69832583, 69832733-69833075, 69833199- |
| | | | | 69874027, 69874167-69875925, 69876024-69905705, 69905835-69921940, 69922153-69942662, 69942754- |
| | | | | 69951610, 69951787-69959331, 69959388-69963349, 69963433-69964031, 69964162-69965053, 69965131- |
| | | | | 69965410, 69965484-69965703, 69965794-69967871, 69968033-69969754, 69969890-69970213, 69970356- |
| | | | | 69971019, 69971142-69971449, 69971556-69972928, 69973027-69973242, 69973317-69973742, |
| 48 | WWP2 | NM_199423 | chr16 | 69796295-69810090, 69810145-69820897, 69820984-69832583, 69832733-69833075, 69833199- |
| | | | | 69874027, 69874167-69875925, 69876024-69905705, 69905835-69921940, 69922153-69942662, |
| 48 | PDXDC2P | NR_003610 | chr16 | 70010636-70010712, 70011907-70011978, 70012016-70016251, 70016398-70016559, 70016661- |
| | | | | 70020282, 70020413-70023857, 70024112-70024498, 70024587-70029970, 70030130-70030291, 70030372- |
| | | | | 70044652, 70044760-70045742, 70045833-70048005, 70048071-70051948, 70052001-70055725, 70055853- |
| | | | | 70057280, 70057381-70057531, 70057582-70058487, 70058574-70064889, 70064970-70065801, 70065872- |
| | | | | 70068072, 70068264-70070333, 70070482-70072806, 70072889-70076252, 70076320-70076831, 70076907- |
| | | | | 70099657, |
| 48 | AARS | NM_001605 | chr16 | 70286810-70287169, 70287285-70287615, 70287704-70287820, 70287942-70288522, 70288638- |
| | | | | 70289629, 70289740-70291934, 70292121-70292881, 70293090-70294945, 70295061-70296247, 70296428- |
| | | | | 70298859, 70299006-70299439, 70299566-70301560, 70301713-70302172, 70302283-70303519, 70303667- |
| | | | | 70304097, 70304244-70305682, 70305876-70310387, 70310535-70310867, 70311058-70316521, 70316688- |
| | | | | 70323322, |
| 48 | DDX19A | NM_018332 | chr16 | 70380952-70384458, 70384509-70389386, 70389439-70390013, 70390151-70395319, 70395414- |
| | | | | 70398444, 70398549-70398918, 70399035-70399986, 70400166-70400525, 70400765-70404124, 70404289- |
| | | | | 70405273, 70405467-70405826, |
| 48 | COG4 | NM_001195139 | chr16 | 70515048-70515260, 70515391-70515665, 70515769-70515993, 70516079-70516630, 70516725- |
| | | | | 70517754, 70517873-70530167, 70530335-70531122, 70531291-70531838, 70531959-70534859, 70534995- |
| | | | | 70542307, 70542368-70543132, 70543292-70543817, 70543925-70546140, 70546336-70548236, 70548413- |
| | | | | 70551527, 70551644-70553550, 70553635-70557274, |
| 48 | COG4 | NM_015386 | chr16 | 70515048-70515260, 70515391-70515665, 70515769-70515993, 70516079-70516630, 70516725- |
| | | | | 70517754, 70517873-70524231, 70524296-70530167, 70530335-70531122, 70531291-70531838, 70531959- |
| | | | | 70534859, 70534995-70542307, 70542368-70543132, 70543292-70543817, 70543925-70546140, 70546336- |
| | | | | 70548236, 70548413-70551527, 70551644-70553550, 70553635-70557274, |
| 48 | SF3B3 | NM_012426 | chr16 | 70557832-70560488, 70560630-70562774, 70563103-70564646, 70564821-70566380, 70566524- |
| | | | | 70569209, 70569324-70572224, 70572364-70573005, 70573111-70575570, 70575738-70578339, 70578437- |
| | | | | 70582271, 70582346-70588347, 70588501-70588952, 70589110-70590080, 70590238-70590787, 70590933- |
| | | | | 70594370, 70594495-70595531, 70595688-70597777, 70597954-70598966, 70599174-70599269, 70599428- |
| | | | | 70601312, 70601440-70602184, 70602399-70602944, 70603045-70603907, 70604053-70604996, 70605103- |
| | | | | 70605574, |
| 48 | MTSS1L | NM_138383 | chr16 | 70698353-70698499, 70698667-70698821, 70699000-70699367, 70699444-70708207, 70708432- |
| | | | | 70710782, 70710882-70711693, 70711803-70712153, 70712313-70713216, 70713227-70713531, 70713608- |
| | | | | 70713687, 70713781-70713872, 70713959-70714695, 70714771-70714865, 70714929-70719624, |
| 49 | KIF4A | NM_012310 | chrX | 69510001-69510286, 69510429-69510539, 69510656-69516846, 69517039-69518961, 69519053- |
| | | | | 69521748, 69521917-69522152, 69522249-69549253, 69549372-69550005, 69550183-69553476, 69553540- |
| | | | | 69561647, 69561782-69563551, 69563612-69563725, 69563833-69572448, 69572507-69573470, 69573658- |
| | | | | 69593999, 69594105-69595052, 69595199-69595948, 69596061-69606466, 69606552-69607032, 69607148- |
| | | | | 69615519, 69615677-69615797, 69615900-69622414, 69622546-69623712, 69623885-69624563, 69624673- |
| | | | | 69625676, 69625756-69626107, 69626205-69626741, 69626926-69637736, 69637855-69639509, 69639634- |
| | | | | 69639910, |
| 49 | PGK1 | NM_000291 | chrX | 77359903-77365362, 77365415-77369239, 77369397-77369511, 77369658-77372807, 77372913- |
| | | | | 77373546, 77373668-77378330, 77378447-77378690, 77378872-77380369, 77380549-77380822, 77380923- |
| | | | | 77381285, |
| 49 | TAF9B | NM_015975 | chrX | 77387271-77388833, 77388946-77392410, 77392488-77393244, 77393381-77393457, 77393596- |
| | | | | 77394338, 77394422-77395056, |
| 49 | PCDH11X | NM_032968 | chrX | 91091044-91131778, 91134273-91456372, 91456455-91518111, 91518143-91642732, 91642933- |
| | | | | 91723296, 91723322-91873261, |
| 49 | PCDH11X | NM_001168360 | chrX | 91091044-91131778, 91134273-91456372, 91456455-91518111, 91518143-91642732, 91642933-91873261, |
| 49 | PCDH11X | NM_032969 | chrX | 91091044-91131778, 91134273-91456372, 91456455-91642732, 91642933-91723296, 91723322-91873261, |
| 49 | PCDH11X | NM_032967 | chrX | 91034684-91065470, 91065578-91066234, 91066295-91090458, 91091044-91131778, 91134273-91137904, |
| 49 | PCDH11X | NM_001168363 | chrX | 91091044-91131778, 91134273-91456372, 91456455-91642732, 91642933-91873261, |
| 49 | PCDH11X | NM_001168362 | chrX | 91091044-91131778, 91134273-91642732, 91642933-91723296, 91723322-91873261, |
| 49 | PCDH11X | NM_001168361 | chrX | 91091044-91131778, 91134273-91456372, 91456455-91518111, 91518143-91542964, 91543002- |
| | | | | 91723296, 91723322-91873261, |
| 49 | PCDH11X | NM_014522 | chrX | 91091044-91131778, |
| 49 | DIAPH2 | NM_006729 | chrX | 95940190-95990755, 95990790-95993583, 95993762-96013151, 96013258-96136576, 96136718- |
| | | | | 96167325, 96167402-96167480, 96167552-96171435, 96171574-96173506, 96173617-96185730, 96185843- |
| | | | | 96192230, 96192351-96194263, 96194382-96197015, 96197136-96200524, 96200591-96203932, 96204039- |
| | | | | 96212825, 96213148-96220090, 96220207-96327938, 96328042-96330164, 96330261-96354691, 96354795- |
| | | | | 96369723, 96369965-96396662, 96396794-96502712, 96502839-96603113, 96603280-96638906, 96639044- |
| | | | | 96684647, 96684745-96854250, |
| 49 | DIAPH2 | NM_007309 | chrX | 95940190-95990755, 95990790-95993583, 95993762-96013151, 96013258-96136576, 96136718- |
| | | | | 96167325, 96167402-96167480, 96167552-96171435, 96171574-96173506, 96173617-96185730, 96185843- |
| | | | | 96192230, 96192351-96194263, 96194382-96197015, 96197136-96200524, 96200591-96203932, 96204039- |
| | | | | 96212825, 96213148-96220090, 96220207-96327938, 96328042-96330164, 96330261-96354691, 96354795- |
| | | | | 96369723, 96369965-96396662, 96396794-96502712, 96502839-96603113, 96603280-96638906, 96639044- |
| | | | | 96684647, 96684745-96724691, |
| 49 | CENPI | NM_006733 | chrX | 100355055-100356045, 100356286-100357261, 100357401-100364460, 100364581- |
| | | | | 100364878, 100364988-100375389, 100375440-100375725, 100375774-100381696, 100381788- |
| | | | | 100382125, 100382256-100382485, 100382655-100383703, 100383826-100384969, 100385063- |
| | | | | 100387177, 100387297-100387379, 100387446-100395653, 100395750-100400051, 100400189- |
| | | | | 100401140, 100401267-100402750, 100402801-100402930, 100403151-100416515, 100416559- |
| | | | | 100417820, |
| 50 | KIAA1609 | NM_020947 | chr16 | 84513633-84514133, 84514315-84516196, 84516317-84520235, 84520666-84522882, 84523029- |
| | | | | 84529287, 84529520-84531538, 84531718-84538205, |
| 50 | USP10 | NM_005153 | chr16 | 84733718-84767039, 84767110-84773913, 84773976-84778237, 84779280-84792320, 84792414- |
| | | | | 84792967, 84793079-84793495, 84793553-84793776, 84793882-84796593, 84796695-84797690, 84797870- |
| | | | | 84801797, 84801965-84806145, 84806292-84808764, 84808832-84812499, |
| 50 | KIAA0182 | NM_001134473 | chr16 | 85645138-85682156, 85682358-85687882, 85688057-85688398, 85688598-85689330, 85689524- |
| | | | | 85689947, 85690272-85690881, 85691211-85694750, 85695372-85696585, 85696700-85696948, 85697221- |
| | | | | 85698619, 85698735-85699580, 85699954-85701744, 85702031-85704599, 85704705-85706009, |
| 50 | KIAA0182 | NM_014615 | chr16 | 85647005-85667518, 85667739-85682156, 85682358-85687882, 85688057-85688398, 85688598- |
| | | | | 85689330, 85689524-85689947, 85690272-85690881, 85691211-85694750, 85695372-85696585, 85696700- |
| | | | | 85696948, 85697221-85698619, 85698735-85699580, 85699954-85701744, 85702031-85704599, 85704705- |
| | | | | 85706009, |
| 50 | COX4I1 | NM_001861 | chr16 | 85833359-85834809, 85834885-85838541, 85838711-85839337, 85839471-85840342, |
| 50 | SLC7A5 | NM_003486 | chr16 | 87866632-87868018, 87868198-87870102, 87870254-87871449, 87871548-87872318, 87872424- |
| | | | | 87873306, 87873432-87874033, 87874080-87874654, 87874762-87885328, 87885456-87902489, |
| 50 | BANP | NM_001173543 | chr16 | 88003777-88008652, 88008792-88014640, 88014734-88017688, 88017866-88037899, 88038018- |
| | | | | 88039694, 88039872-88052032, 88052274-88061087, 88061257-88066713, 88066852-88068936, 88069001- |
| | | | | 88071577, 88071618-88098871, 88098939-88105673, 88105819-88110194, |
| 50 | BANP | NM_079837 | chr16 | 87985122-88008652, 88008792-88014640, 88014734-88017664, 88017866-88039694, 88039872- |
| | | | | 88052032, 88052274-88061087, 88061257-88066713, 88066852-88068927, 88069001-88071577, 88071618- |
| | | | | 88098871, 88098939-88105673, 88105819-88110194, |
| 50 | BANP | NM_017869 | chr16 | 87985122-88008652, 88008792-88014640, 88014734-88017664, 88017866-88039694, 88039872- |
| | | | | 88052032, 88052274-88061087, 88061257-88066713, 88066852-88068927, 88069001-88071577, 88071618- |
| | | | | 88105673, 88105819-88110194, |
| 50 | BANP | NM_001173542 | chr16 | 87993600-88008652, 88008792-88014640, 88014734-88017664, 88017866-88037899, 88038018- |
| | | | | 88039694, 88039872-88052032, 88052274-88061087, 88061257-88066713, 88066852-88068927, 88069001- |
| | | | | 88071577, 88071618-88105673, 88105819-88110194, |
| 50 | BANP | NM_001173541 | chr16 | 87985122-88008652, 88008792-88014640, 88014734-88017664, 88017866-88039694, 88039872- |
| | | | | 88052032, 88052274-88061087, 88061257-88066713, 88066852-88068936, 88069001-88071577, 88071618- |
| | | | | 88105673, 88105819-88110194, |
| 50 | BANP | NM_001173539 | chr16 | 87985122-88008652, 88008792-88014640, 88014734-88017664, 88017866-88037899, 88038018- |
| | | | | 88039694, 88039872-88052032, 88052274-88061087, 88061257-88066713, 88066852-88068936, 88069001- |
| | | | | 88071577, 88071618-88105673, 88105819-88110194, |
| 50 | BANP | NM_001173540 | chr16 | 87985122-88008652, 88008810-88014640, 88014734-88017664, 88017866-88039694, 88039872- |
| | | | | 88052032, 88052274-88061087, 88061257-88066713, 88066852-88068927, 88069001-88071577, 88071618- |
| | | | | 88098871, 88098939-88105673, 88105819-88110194, |
| 50 | ZC3H18 | NM_144604 | chr16 | 88636975-88643516, 88644135-88653006, 88653093-88664584, 88664735-88665026, 88665121- |
| | | | | 88666197, 88666357-88675340, 88675460-88677674, 88677945-88688603, 88688797-88689625, 88689753- |
| | | | | 88690364, 88690471-88691008, 88691154-88691608, 88691676-88694028, 88694191-88694326, 88694528- |
| | | | | 88695164, 88695263-88696891, 88696990-88697507, |
| 50 | IL17C | NM_013278 | chr16 | 88705056-88705387, 88705718-88706220, |
| 50 | MVD | NM_002461 | chr16 | 88719014-88719706, 88719817-88721098, 88721216-88721605, 88721826-88722062, 88722139- |
| | | | | 88722511, 88722713-88723842, 88723991-88724321, 88724438-88725056, 88725129-88729417, |
| 50 | PABPN1L | NM_001080487 | chr15 | 88930204-88930643, 88930788-88931259, 88931349-88931428, 88931537-88931969, 88932039- |
| | | | | 88932212, 88932350-88932758, |
| 50 | ANKRD11 | NM_013275 | chr16 | 89335072-89337223, 89337318-89341220, 89341366-89341499, 89341600-89345478, 89352058- |
| | | | | 89352445, 89352595-89354934, 89355079-89357031, 89357237-89357419, 89357592-89371612, 89371753- |
| | | | | 89383339, 89383487-89484690, 89484777-89556651, |
| 50 | LOC100128881 | NR_036480 | chr16 | 89778631-89779095, 89779227-89782877, 89783313-89783750, |
| 50 | FANCA | NM_000135 | chr16 | 89805117-89805288, 89805383-89805539, 89805698-89805884, 89805962-89806400, 89806508- |
| | | | | 89807210, 89807275-89809206, 89809347-89811365, 89811480-89812990, 89813097-89813237, 89813299- |
| | | | | 89815065, 89815176-89816136, 89816311-89818544, 89818631-89824983, 89825114-89828355, 89828431- |
| | | | | 89831296, 89831475-89833547, 89833646-89836243, 89836433-89836572, 89836668-89836970, 89837043- |
| | | | | 89838084, 89838223-89839677, 89839793-89842148, 89842224-89845207, 89845259-89845349, 89845412- |
| | | | | 89846275, 89846366-89849265, 89849327-89849413, 89849511-89851260, 89851373-89857809, 89857945- |
| | | | | 89858333, 89858477-89858877, 89858956-89862312, 89862427-89865572, 89865641-89866011, 89866047- |
| | | | | 89869665, 89869750-89871686, 89871801-89874700, 89874776-89877113, 89877211-89877335, 89877480- |
| | | | | 89880926, 89881022-89882283, 89882395-89882943, |
| 50 | FANCA | NM_001018112 | chr16 | 89865488-89865572, 89865641-89866011, 89866047-89869665, 89869750-89871686, 89871801- |
| | | | | 89874700, 89874776-89877113, 89877211-89877335, 89877480-89880926, 89881022-89882283, 89882395- |
| | | | | 89882943, |
| 50 | LOC100130015 | NR_027336 | chr16 | 90110398-90110908, 90111032-90113363, 90113505-90114149, |
| 50 | LOC100130015 | NR_027335 | chr16 | 90106826-90110054, 90110398-90110908, 90111032-90113363, 90113505-90113944, |
| 51 | SRD5A1 | NM_001047 | chr5 | 6633983-6651953, 6652122-6656189, 6656293-6662927, 6663080-6668313, |
| 51 | PAPD7 | NM_006999 | chr5 | 6714813-6737621, 6737747-6738794, 6738843-6739843, 6739966-6742601, 6742711-6743823, 6743954- |
| | | | | 6746325, 6746541-6748575, 6748704-6749668, 6749771-6750442, 6750617-6751150, 6751311- |
| | | | | 6752984, 6753151-6754862, |
| 51 | PAPD7 | NM_001171806 | chr5 | 6746541-6748575, 6748704-6749668, 6749771-6750442, 6750617-6751150, 6751311-6752984, 6753151- |
| | | | | 6754862, |
| 51 | PAPD7 | NM_001171805 | chr5 | 6714813-6737621, 6737747-6738794, 6738843-6739843, 6739966-6742601, 6742711-6743823, 6743954- |
| | | | | 6746325, 6746541-6748575, 6748704-6749668, 6749771-6750442, 6750617-6751150, 6751311- |
| | | | | 6752984, 6753151-6754865, |
| 51 | MTRR | NM_024010 | chr5 | 7869303-7870881, 7871037-7873484, 7873640-7875369, 7875489-7878055, 7878436-7883266, 7883391- |
| | | | | 7885812, 7885968-7886726, 7886817-7889206, 7889389-7891483, 7891528-7892838, 7893027- |
| | | | | 7895845, 7895966-7896975, 7897070-7897176, 7897361-7900025, |
| 51 | MTRR | NM_002454 | chr5 | 7869329-7870881, 7871037-7873484, 7873640-7875369, 7875489-7878055, 7878436-7883266, 7883391- |
| | | | | 7885812, 7885968-7886726, 7886817-7889206, 7889389-7891483, 7891528-7892838, 7893027- |
| | | | | 7895845, 7895966-7896975, 7897070-7897176, 7897361-7900025, |
| 51 | MARCH6 | NM_005885 | chr5 | 10354030-10377908, 10378007-10378869, 10378945-10381910, 10382056-10387104, 10387179- |
| | | | | 10390442, 10390613-10391652, 10391844-10394192, 10394256-10394863, 10394898-10397403, 10397457- |
| | | | | 10400894, 10400955-10402169, 10402252-10402494, 10402565-10402643, 10402720-10403517, 10403654- |
| | | | | 10405668, 10405790-10407212, 10407315-10410249, 10410389-10411443, 10411650-10414543, 10414615- |
| | | | | 10415598, 10415782-10417380, 10417517-10423845, 10423937-10426500, 10426635-10430003, 10430141- |
| | | | | 10433704, |
| 51 | DAP | NM_004394 | chr5 | 10681282-10683639, 10683684-10748285, 10748384-10761124, |
| 51 | DROSHA | NM_013235 | chr5 | 31401670-31405782, 31405831-31406958, 31407053-31409161, 31409267-31409355, 31409440- |
| | | | | 31410851, 31410995-31421377, 31421485-31422892, 31423052-31424532, 31424579-31429580, 31429653- |
| | | | | 31431681, 31431786-31435870, 31435972-31437344, 31437406-31448652, 31448715-31449386, 31449527- |
| | | | | 31451638, 31451748-31464341, 31464451-31466287, 31466389-31468044, 31468171-31472168, 31472340- |
| | | | | 31483659, 31483736-31484986, 31485070-31486596, 31486670-31493312, 31493401-31495391, 31495480- |
| | | | | 31504660, 31504743-31508726, 31508883-31511140, 31511284-31515093, 31515327-31515559, 31515672- |
| | | | | 31521228, 31521323-31526184, 31527020-31529145, 31529213-31530903, 31531032-31532095, |
| 51 | DROSHA | NM_001100412 | chr5 | 31401670-31405782, 31405831-31406958, 31407053-31409161, 31409267-31409355, 31409440- |
| | | | | 31410851, 31410995-31421377, 31421485-31422892, 31423052-31424532, 31424579-31429580, 31429653- |
| | | | | 31431681, 31431786-31435870, 31435972-31437344, 31437406-31448652, 31448715-31449386, 31449527- |
| | | | | 31451638, 31451748-31464341, 31464451-31466287, 31466389-31468044, 31468171-31472168, 31472340- |
| | | | | 31483659, 31483736-31484986, 31485070-31486596, 31486670-31493312, 31493401-31495391, 31495480- |
| | | | | 31504660, 31504743-31508726, 31508883-31511140, 31511284-31515093, 31515327-31521228, 31521323- |
| | | | | 31526184, 31527020-31529145, 31529213-31530903, 31531032-31531555, 31531633-31532095, |
| 51 | C5orf22 | NM_018356 | chr5 | 31532581-31534377, 31534525-31535849, 31536001-31538365, 31538797-31541054, 31541119- |
| | | | | 31541386, 31541510-31545751, 31545820-31551398, 31551540-31552878, |
| 51 | GOLPH3 | NM_022130 | chr5 | 32126743-32135676, 32135793-32143853, 32143987-32173914, |
| 51 | ZFR | NM_016107 | chr5 | 32356046-32364052, 32364152-32364268, 32364382-32379219, 32379317-32380179, 32380279- |
| | | | | 32385612, 32385756-32387653, 32387806-32388573, 32388781-32390379, 32390544-32395263, 32395411- |
| | | | | 32397323, 32397445-32400110, 32400309-32403209, 32403503-32404009, 32404203-32406877, 32407127- |
| | | | | 32415072, 32415293-32417751, 32417898-32419924, 32420209-32444333, 32444435-32444726, |
| 51 | TARS | NM_152295 | chr5 | 33441250-33445428, 33445511-33448645, 33448838-33453392, 33453518-33455048, 33455172- |
| | | | | 33455690, 33455810-33456105, 33456172-33456252, 33456333-33457360, 33457509-33458669, 33458770- |
| | | | | 33459798, 33459967-33461005, 33461170-33461261, 33461401-33461770, 33461850-33462009, 33462112- |
| | | | | 33462202, 33462309-33463856, 33463931-33466974, 33467091-33467663, |
| 52 | PPP1CB | NM_002709 | chr2 | 28975043-28999715, 28999849-29001673, 29001906-29004602, 29004709-29006771, 29006845- |
| | | | | 29011522, 29011676-29016727, 29016864-29022063, |
| 52 | PPP1CB | NM_206876 | chr2 | 28974687-28974902, 28975043-28999715, 28999849-29001673, 29001906-29004602, 29004709- |
| | | | | 29006771, 29006845-29011522, 29011676-29016727, 29016864-29022063, |
| 52 | GALNT14 | NM_024572 | chr2 | 31133926-31135087, 31135209-31146983, 31147130-31147604, 31147690-31152259, 31152354- |
| | | | | 31154932, 31155061-31165065, 31165171-31167722, 31167809-31168647, 31168737-31178482, 31178606- |
| | | | | 31178776, 31178844-31181261, 31181331-31189068, 31189169-31215702, 31215874-31360822, |
| 52 | MEMO1 | NM_015955 | chr2 | 32093562-32094915, 32095022-32108453, 32108532-32117059, 32117204-32142993, 32143107- |
| | | | | 32145865, 32145980-32157134, 32157205-32168369, 32168453-32235254, |
| 52 | MEMO1 | NM_001137602 | chr2 | 32093562-32094915, 32095022-32108453, 32108532-32117059, 32117204-32142993, 32143107- |
| | | | | 32145865, 32145980-32168369, 32168453-32235254, |
| 52 | BIRC6 | NM_016252 | chr2 | 32582555-32602654, 32602838-32605219, 32605359-32613816, 32614012-32617105, 32617219- |
| | | | | 32620577, 32620662-32626229, 32626454-32626529, 32626692-32631565, 32631626-32639835, 32641232- |
| | | | | 32654212, 32654364-32655931, 32656159-32658710, 32658873-32660562, 32660654-32661119, 32661253- |
| | | | | 32664574, 32664755-32666395, 32666531-32667131, 32667295-32667390, 32667524-32668557, 32668657- |
| | | | | 32670587, 32670737-32673861, 32673997-32678874, 32679011-32688260, 32688450-32689575, 32689892- |
| | | | | 32690131, 32690233-32692591, 32692808-32692969, 32693180-32693503, 32693852-32694461, 32694683- |
| | | | | 32695234, 32695370-32696080, 32696193-32698178, 32698308-32701279, 32701413-32702434, 32702652- |
| | | | | 32703701, 32703887-32704528, 32704686-32706386, 32706522-32707495, 32707651-32710708, 32710827- |
| | | | | 32712712, 32712869-32713653, 32713782-32715106, 32715219-32716490, 32716626-32718605, 32718735- |
| | | | | 32724612, 32725177-32726778, 32726956-32727860, 32727959-32728107, 32728304-32730070, 32730273- |
| | | | | 32733045, 32733327-32734803, 32735062-32735591, 32735702-32737998, 32738222-32740055, 32740838- |
| | | | | 32743319, 32743465-32743882, 32744014-32749953, 32750087-32750529, 32750696-32754716, 32754892- |
| | | | | 32756420, 32756619-32768306, 32768609-32770708, 32770928-32772915, 32773082-32774378, 32774549- |
| | | | | 32800221, 32800434-32818980, 32819128-32820099, 32820212-32822816, 32823036-32824804, 32824968- |
| | | | | 32828071, 32828151-32832520, 32832711-32836513, 32836650-32842790, |
| 52 | FAM98A | NM_015475 | chr2 | 33810512-33810595, 33810765-33811627, 33811746-33812305, 33812388-33813400, 33813587- |
| | | | | 33817145, 33817282-33820554, 33820705-33824234, |
| 52 | ATL2 | NM_022374 | chr2 | 38523733-38525284, 38525718-38526396, 38526470-38527412, 38527471-38536519, 38536649- |
| | | | | 38537449, 38537590-38540291, 38540386-38541748, 38541807-38542424, 38542477-38545675, 38545782- |
| | | | | 38546025, 38546162-38570408, 38570655-38604283, |
| 52 | ATL2 | NM_001135673 | chr2 | 38523256-38525284, 38525718-38526396, 38526470-38527412, 38527471-38536519, 38536649- |
| | | | | 38537449, 38537590-38540291, 38540386-38541748, 38541807-38542424, 38542477-38545675, 38545782- |
| | | | | 38546025, 38546162-38570408, 38570655-38604283, |
| 52 | ATL2 | NR_024191 | chr2 | 38523256-38525284, 38525718-38526396, 38526470-38527412, 38527471-38536519, 38536649- |
| | | | | 38537449, 38537590-38540291, 38540386-38541748, 38541807-38542424, 38542477-38545675, 38545782- |
| | | | | 38546025, 38546162-38604283, |
| 52 | EML4 | NM_001145076 | chr2 | 42396777-42472643, 42472828-42483639, 42483771-42490316, 42490447-42491844, 42491872- |
| | | | | 42507988, 42508114-42509961, 42510113-42511772, 42511844-42513407, 42513520-42515365, 42515463- |
| | | | | 42522263, 42522400-42522519, 42522657-42528379, 42528533-42530242, 42530370-42530453, 42530587- |
| | | | | 42531622, 42531692-42543100, 42543191-42544565, 42544665-42552605, 42552695-42553292, 42553393- |
| | | | | 42556024, 42556157-42556872, |
| 52 | EML4 | NM_019063 | chr2 | 42396777-42472643, 42472828-42483639, 42483771-42488259, 42488435-42490316, 42490447- |
| | | | | 42491844, 42491872-42507988, 42508114-42509961, 42510113-42511772, 42511844-42513407, 42513520- |
| | | | | 42515365, 42515463-42522263, 42522400-42522519, 42522657-42528379, 42528533-42530242, 42530370- |
| | | | | 42530453, 42530587-42531622, 42531692-42543100, 42543191-42544565, 42544665-42552605, 42552695- |
| | | | | 42553292, 42553393-42556024, 42556157-42556872, |
| 52 | LRPPRC | NM_133259 | chr2 | 44115796-44116871, 44117016-44121682, 44121769-44123771, 44123848-44126356, 44126474- |
| | | | | 44126603, 44126745-44128457, 44128664-44132829, 44132920-44139569, 44139698-44145162, 44145273- |
| | | | | 44145393, 44145538-44152204, 44152297-44153030, 44153101-44161327, 44161436-44161891, 44162018- |
| | | | | 44170824, 44171034-44172469, 44172557-44173250, 44173383-44174394, 44174510-44174868, 44174915- |
| | | | | 44175259, 44175339-44175549, 44175658-44176739, 44176799-44177710, 44177740-44184522, 44184591- |
| | | | | 44187678, 44187774-44190725, 44190846-44200744, 44200854-44200932, 44201040-44201287, 44201435- |
| | | | | 44201751, 44201898-44202228, 44202357-44203280, 44203369-44204131, 44204192-44204292, 44204416- |
| | | | | 44206963, 44207088-44209375, 44209574-44222936, |
| 52 | PPM1B | NM_177969 | chr2 | 44396414-44436347, 44436467-44445104, 44445218-44445617, 44445677-44457550, |
| 52 | PPM1B | NM_001033556 | chr2 | 44396414-44428323, 44429185-44436347, |
| 52 | PPM1B | NM_177968 | chr2 | 44396414-44428323, 44429185-44436347, 44436467-44445104, 44445218-44445617, 44445677-44459453, |
| 52 | PPM1B | NM_002706 | chr2 | 44396414-44428323, 44429185-44436347, 44436467-44445104, 44445218-44445617, 44445677-44457550, |
| 52 | PPM1B | NM_001033557 | chr2 | 44396414-44428323, 44429185-44436347, 44436467-44445104, 44445218-44445617, 44445677-44457130, |
| 52 | C2orf34 | NM_024766 | chr2 | 44589285-44599853, 44600028-44617378, 44617445-44931420, 44931483-44933424, 44933481- |
| | | | | 44934536, 44934602-44942424, 44942493-44970759, 44970836-44981192, 44981258-44993567, 44993701- |
| | | | | 44999173, |
| 52 | SIX2 | NM_016932 | chr2 | 45233625-45235688, |
| 52 | SRBD1 | NM_018079 | chr2 | 45616739-45620082, 45620269-45640251, 45640433-45645502, 45645681-45646925, 45647034- |
| | | | | 45704130, 45704215-45715377, 45715471-45773869, 45773979-45774659, 45774752-45778262, 45778422- |
| | | | | 45780760, 45780870-45789790, 45789896-45800344, 45800482-45801764, 45801863-45807012, 45807153- |
| | | | | 45808830, 45808950-45812745, 45812914-45826586, 45826975-45829040, 45829223-45832499, 45832581- |
| | | | | 45838356, |
| 52 | PSME4 | NM_014614 | chr2 | 54092711-54093220, 54093361-54093882, 54094018-54096511, 54096676-54101474, 54101632- |
| | | | | 54102762, 54102893-54112825, 54112913-54114394, 54114605-54114993, 54115127-54115856, 54115950- |
| | | | | 54117239, 54117346-54119943, 54120098-54120809, 54120937-54122142, 54122214-54122718, 54122874- |
| | | | | 54123939, 54124023-54125005, 54125137-54125436, 54125492-54127016, 54127155-54128485, 54128697- |
| | | | | 54131160, 54131294-54133733, 54133831-54133941, 54134006-54135455, 54135556-54135645, 54135703- |
| | | | | 54135919, 54135978-54137210, 54137268-54146286, 54146384-54147328, 54147488-54148024, 54148242- |
| | | | | 54149652, 54149722-54150184, 54150256-54150456, 54150557-54152674, 54152827-54153094, 54153161- |
| | | | | 54154529, 54154621-54155252, 54155441-54158970, 54159238-54159803, 54159898-54161740, 54161865- |
| | | | | 54163222, 54163299-54163899, 54163965-54164526, 54164678-54167091, 54167138-54175556, 54175675- |
| | | | | 54176278, 54176421-54197678, |
| 53 | PINK1 | NM_032409 | chr1 | 20960429-20964333, 20964623-20966383, 20966486-20970981, 20971166-20972051, 20972217- |
| | | | | 20974996, 20975126-20975486, 20975725-20976925, |
| 53 | NBPF3 | NM_032264 | chr1 | 21766842-21771439, 21771713-21795179, 21795391-21797123, 21797228-21798060, 21798277- |
| | | | | 21799334, 21799409-21799871, 21800079-21801391, 21801445-21804635, 21804801-21805840, 21805894- |
| | | | | 21806542, 21806717-21807421, 21807475-21808088, 21808263-21808975, 21809029-21809634, |
| 53 | HSPG2 | NM_005529 | chr1 | 22149982-22150107, 22150213-22150612, 22150698-22150797, 22150870-22151028, 22151109- |
| | | | | 22151195, 22151273-22154354, 22154413-22154521, 22154646-22154746, 22154920-22155326, 22155573- |
| | | | | 22155874, 22156098-22156484, 22156585-22157473, 22157584-22157706, 22157818-22157954, 22158056- |
| | | | | 22158143, 22158290-22158986, 22159100-22159759, 22159878-22159958, 22160108-22160307, 22160419- |
| | | | | 22161170, 22161439-22162031, 22162131-22163293, 22163500-22165316, 22165579-22165862, 22166044- |
| | | | | 22166313, 22166511-22167592, 22167779-22168030, 22168167-22168493, 22168636-22168730, 22168874- |
| | | | | 22169261, 22169415-22169788, 22169932-22170639, 22170793-22172599, 22172749-22172939, 22173093- |
| | | | | 22173845, 22173986-22174180, 22174334-22174449, 22174587-22175134, 22175288-22175384, 22175525- |
| | | | | 22176532, 22176686-22176854, 22176992-22178037, 22178191-22178282, 22178420-22178579, 22178727- |
| | | | | 22179191, 22179326-22179410, 22179564-22180684, 22180837-22181102, 22181259-22181339, 22181477- |
| | | | | 22181795, 22181940-22182014, 22182169-22182278, 22182406-22183506, 22183689-22183776, 22183879- |
| | | | | 22186057, 22186170-22186326, 22186496-22186668, 22186729-22188248, 22188337-22188479, 22188609- |
| | | | | 22190591, 22190707-22191334, 22191567-22191776, 22191859-22192208, 22192303-22198677, 22198871- |
| | | | | 22199111, 22199254-22199488, 22199585-22199866, 22199918-22200416, 22200505-22200897, 22201027- |
| | | | | 22201107, 22201223-22201382, 22201496-22202120, 22202241-22202354, 22202536-22202720, 22202899- |
| | | | | 22203003, 22203146-22204677, 22204747-22204924, 22204985-22205068, 22205157-22205485, 22205615- |
| | | | | 22206598, 22206748-22206854, 22207053-22207147, 22207329-22207830, 22207996-22211019, 22211168- |
| | | | | 22211258, 22211412-22211504, 22211651-22211811, 22211945-22213706, 22213828-22213911, 22214168- |
| | | | | 22214429, 22214560-22216472, 22216635-22216918, 22216979-22217076, 22217188-22222413, 22222460- |
| | | | | 22222666, 22222804-22263646, |
| 53 | TCEA3 | NM_003196 | chr1 | 23707968-23710837, 23710911-23713764, 23713913-23720370, 23720525-23724034, 23724095- |
| | | | | 23724283, 23724449-23735155, 23735220-23743740, 23743884-23744395, 23744503-23745568, 23745633- |
| | | | | 23751056, |
| 53 | HMGCL | NM_000191 | chr1 | 24129055-24130888, 24131016-24134623, 24134814-24137224, 24137290-24140678, 24140829- |
| | | | | 24143163, 24143261-24143964, 24144074-24146998, 24147084-24151844, |
| 53 | HMGCL | NM_001166059 | chr1 | 24129055-24130888, 24131016-24134623, 24134814-24143163, 24143261-24143964, 24144074- |
| | | | | 24146998, 24147084-24151844, |
| 53 | MYOM3 | NM_152372 | chr1 | 24384118-24385412, 24385470-24387548, 24387573-24387761, 24387800-24388434, 24388600- |
| | | | | 24389614, 24389725-24389980, 24390021-24390560, 24390650-24392379, 24392492-24393509, 24393579- |
| | | | | 24394458, 24394520-24394711, 24394858-24397605, 24397744-24398457, 24398504-24400648, 24400760- |
| | | | | 24401807, 24401966-24402647, 24402764-24406504, 24406685-24407818, 24407947-24408489, 24408679- |
| | | | | 24409081, 24409205-24410956, 24411130-24413132, 24413318-24416026, 24416156-24416553, 24416609- |
| | | | | 24417285, 24417487-24418662, 24418806-24419435, 24419599-24421341, 24421481-24421930, 24421977- |
| | | | | 24424411, 24424502-24426168, 24426266-24432408, 24432568-24433561, 24433723-24434481, 24434564- |
| | | | | 24434964, 24435145-24438519, |
| 53 | PAQR7 | NM_178422 | chr1 | 26190353-26197098, |
| 53 | UBXN11 | NM_183008 | chr1 | 26609061-26609138, 26609281-26609353, 26609429-26610631, 26610737-26610851, 26610974- |
| | | | | 26611953, 26612033-26612312, 26612529-26620694, 26620823-26623408, 26623487-26623916, 26623973- |
| | | | | 26624451, 26624554-26627415, 26627516-26628183, 26628214-26629302, 26629410-26629532, 26629659- |
| | | | | 26633081, |
| 53 | UBXN11 | NM_145345 | chr1 | 26609061-26609138, 26609281-26609353, 26609429-26610631, 26610737-26610851, 26610974- |
| | | | | 26611953, 26612033-26612312, 26612529-26620694, 26620823-26623408, 26623487-26623916, 26623973- |
| | | | | 26624451, 26624554-26628183, 26628214-26629302, 26629410-26633081, 26633196-26644536, |
| 53 | UBXN11 | NM_001077262 | chr1 | 26609061-26609138, 26609281-26609353, 26609429-26610631, 26610737-26610851, 26610974- |
| | | | | 26611953, 26612033-26612312, 26612529-26627415, 26627516-26628183, 26628214-26629302, 26629410- |
| | | | | 26633081, |
| 54 | C14orf50 | NM_172365 | chr14 | 65016785-65017637, 65017704-65019530, 65019580-65031467, 65031556-65032073, 65032173- |
| | | | | 65032342, 65032411-65035065, 65035166-65041171, 65041308-65053553, 65053598-65053910, 65054091- |
| | | | | 65054820, 65055014-65055868, |
| 54 | TMEM229B | NM_182526 | chr14 | 67940659-67953715, 67953890-67981801, |
| 54 | EXD2 | NM_001193360 | chr14 | 69658309-69670632, 69670718-69676139, 69676521-69695531, 69695790-69697187, 69697316- |
| | | | | 69701415, 69701749-69702761, 69702870-69703122, 69703260-69704290, 69704649-69707599, |
| 54 | EXD2 | NR_034165 | chr14 | 69658757-69670632, 69670718-69676139, 69676521-69676827, 69676919-69695531, 69695790- |
| | | | | 69697187, 69697316-69701415, 69701749-69702761, 69702870-69703122, 69703260-69704290, 69704649- |
| | | | | 69707599, |
| 54 | EXD2 | NR_034164 | chr14 | 69658309-69670632, 69670718-69676139, 69676521-69676827, 69676919-69695531, 69695790- |
| | | | | 69697187, 69697316-69701415, 69701749-69702761, 69702870-69703122, 69703260-69704290, 69704649- |
| | | | | 69707599, |
| 54 | EXD2 | NM_001193361 | chr14 | 69658309-69676139, 69676521-69695531, 69695790-69697187, 69697316-69701415, 69701749- |
| | | | | 69702761, 69702870-69703122, 69703260-69704290, 69704649-69707599, |
| 54 | EXD2 | NM_018199 | chr14 | 69658309-69695531, 69695790-69697187, 69697316-69701415, 69701749-69702761, 69702870- |
| | | | | 69703122, 69703260-69704290, 69704649-69707599, |
| 54 | EXD2 | NM_001193363 | chr14 | 69658757-69676139, 69676521-69695531, 69695790-69697187, 69697316-69701415, 69701749- |
| | | | | 69702761, 69702870-69703122, 69703260-69704290, 69704649-69707599, |
| 54 | EXD2 | NM_001193362 | chr14 | 69658757-69670632, 69670718-69676139, 69676521-69695531, 69695790-69697187, 69697316- |
| | | | | 69701415, 69701749-69702761, 69702870-69703122, 69703260-69704290, 69704649-69707599, |
| 54 | GALNTL1 | NM_001168368 | chr14 | 69727185-69787426, 69787586-69791407, 69791508-69792052, 69792122-69792677, 69792745- |
| | | | | 69795165, 69795289-69798179, 69798269-69799800, 69799887-69800212, 69800318-69805366, 69805495- |
| | | | | 69806242, 69806337-69808396, 69808482-69813755, 69813899-69814592, 69814720-69818746, |
| 54 | GALNTL1 | NM_020692 | chr14 | 69727185-69787426, 69787586-69791407, 69791508-69792052, 69792122-69792677, 69792745- |
| | | | | 69795165, 69795289-69798179, 69798269-69799800, 69799887-69800212, 69800318-69805366, 69805495- |
| | | | | 69806242, 69806337-69808396, 69808482-69813755, 69813899-69814592, 69814720-69818746, 69818888- |
| | | | | 69820047, |
| 54 | DNAL1 | NM_031427 | chr14 | 74111746-74121548, 74121589-74125548, 74125660-74128688, 74128746-74138243, 74138301- |
| | | | | 74153960, 74154089-74156076, 74156219-74162603, |
| 54 | EIF2B2 | NM_014239 | chr14 | 75469857-75469976, 75470099-75470252, 75470403-75471438, 75471604-75472567, 75472665- |
| | | | | 75473278, 75473418-75474423, 75474492-75475732, |
| 54 | KIAA1737 | NM_033426 | chr14 | 77564705-77571998, 77572188-77576153, 77576325-77579766, |
| 54 | POMT2 | NM_013382 | chr14 | 77743825-77744735, 77744852-77745070, 77745213-77746164, 77746272-77746362, 77746424- |
| | | | | 77746733, 77746807-77750138, 77750217-77751291, 77751385-77751822, 77751976-77753085, 77753166- |
| | | | | 77755103, 77755175-77757655, 77757724-77762505, 77762617-77765030, 77765115-77765796, 77765905- |
| | | | | 77767431, 77767593-77769176, 77769287-77771033, 77771144-77772678, 77772785-77778290, 77778377- |
| | | | | 77786775, |
| 55 | MOV10 | NM_001130079 | chr1 | 113217253-113217468, 113217672-113231555, 113231761-113232035, 113232273- |
| | | | | 113232460, 113232721-113234285, 113234422-113235381, 113235552-113236638, 113236795- |
| | | | | 113237073, 113237252-113237369, 113237519-113238036, 113238197-113238768, 113238874- |
| | | | | 113239057, 113239157-113239250, 113239469-113240614, 113240734-113240907, 113241101- |
| | | | | 113241335, 113241412-113242305, 113242433-113242514, 113242605-113242839, 113242963- |
| | | | | 113243036, |
| 55 | MOV10 | NM_020963 | chr1 | 113217352-113217452, 113217672-113231555, 113231761-113232035, 113232273- |
| | | | | 113232460, 113232721-113234285, 113234422-113235381, 113235552-113236638, 113236795- |
| | | | | 113237073, 113237252-113237369, 113237519-113238036, 113238197-113238768, 113238874- |
| | | | | 113239057, 113239157-113239250, 113239469-113240614, 113240734-113240907, 113241101- |
| | | | | 113241335, 113241412-113242305, 113242433-113242514, 113242605-113242839, 113242963- |
| | | | | 113243036, |
| 55 | PHTF1 | NM_006608 | chr1 | 114240348-114240882, 114241010-114242324, 114242422-114243414, 114243572- |
| | | | | 114246701, 114246790-114247286, 114247420-114248510, 114248785-114249214, 114249345- |
| | | | | 114252874, 114253098-114254366, 114254458-114254560, 114254736-114255899, 114256061- |
| | | | | 114267379, 114267516-114269038, 114269197-114280730, 114280891-114281350, 114281422- |
| | | | | 114300319, 114300378-114301279, |
| 55 | SYT6 | NM_205848 | chr1 | 114634705-114636355, 114636426-114640347, 114640500-114641714, 114641888- |
| | | | | 114646221, 114646344-114680115, 114680676-114682235, 114682503-114696230, |
| 55 | ATP1A1OS | NR_027646 | chr1 | 116935649-116943516, 116943754-116961079, |
| 55 | ATP1A1OS | NR_027645 | chr1 | 116941937-116943516, 116943754-116961079, |
| 55 | ATP1A1OS | NR_024124 | chr1 | 116948771-116961079, |
| 55 | ATP1A1OS | NR_024126 | chr1 | 116948771-116953881, 116953938-116961079, |
| 55 | ATP1A1OS | NR_024125 | chr1 | 116948771-116949548, 116949818-116961079, |
| 55 | TRIM45 | NM_001145635 | chr1 | 117655076-117655979, 117656108-117658195, 117658312-117659236, 117659368- |
| | | | | 117660708, 117661390-117663334, |
| 55 | TRIM45 | NM_025188 | chr1 | 117655076-117655979, 117656108-117658195, 117658312-117659236, 117659368- |
| | | | | 117660654, 117661390-117663334, |
| 55 | SPAG17 | NM_206996 | chr1 | 118496673-118506420, 118506562-118509230, 118509377-118512677, 118512740- |
| | | | | 118514484, 118514671-118516046, 118516185-118523891, 118524025-118526432, 118526537- |
| | | | | 118527304, 118527406-118530455, 118530505-118530726, 118530828-118533482, 118533530- |
| | | | | 118534036, 118534152-118535087, 118535272-118537027, 118537154-118538991, 118539124- |
| | | | | 118539219, 118539367-118548035, 118548226-118550665, 118550822-118554849, 118554996- |
| | | | | 118558586, 118558806-118565925, 118566087-118567859, 118568040-118570895, 118571018- |
| | | | | 118574313, 118574491-118579391, 118579507-118581913, 118582068-118583351, 118583465- |
| | | | | 118584424, 118584666-118596623, 118596717-118598354, 118598538-118609366, 118609523- |
| | | | | 118616475, 118616591-118617160, 118617224-118623722, 118623943-118624036, 118624266- |
| | | | | 118628543, 118628696-118629292, 118629408-118629492, 118629632-118634227, 118634342- |
| | | | | 118634551, 118634626-118635777, 118635941-118640291, 118640475-118642227, 118642424- |
| | | | | 118644361, 118644550-118657931, 118658065-118693164, 118693253-118693850, 118693993- |
| | | | | 118727692, |
| 55 | WARS2 | NM_201263 | chr1 | 119576012-119576716, 119576837-119584885, 119584973-119588203, 119588286- |
| | | | | 119618971, 119619231-119683176, |
| 55 | WARS2 | NM_015836 | chr1 | 119575983-119576716, 119576837-119584885, 119584973-119588203, 119588286- |
| | | | | 119618971, 119619231-119683176, |
| 56 | HEATR2 | NM_017802 | chr7 | 766953-769298, 769485-780454, 780581-780982, 781103-794224, 794459-796417, 796632-801388, 801534- |
| | | | | 803441, 803612-810106, 810256-813683, 813836-814641, 814800-819588, 819782-825152, |
| 56 | SUN1 | NM_001171945 | chr7 | 856311-856915, 857137-872140, 872239-878433, 878624-881581, 881768-882841, 882870-882976, |
| 56 | SUN1 | NM_001171946 | chr7 | 855241-872140, 872239-878433, 878624-881581, 881768-882841, 882870-882976, |
| 56 | SUN1 | NM_001171944 | chr7 | 856311-872140, 872239-878433, 878624-881581, 881768-891585, 891681-892244, 892305-892448, 892590- |
| | | | | 893044, 893256-894555, 894644-895993, 896039-897463, 897595-899763, 900020-901015, 901102- |
| | | | | 905587, 905705-908984, 909154-912105, 912200-912850, |
| 56 | SUN1 | NM_025154 | chr7 | 856311-878433, 878624-881581, 881768-882841, 882870-882976, 883158-891585, 891681-892244, 892305- |
| | | | | 892445, 892590-893044, 893256-894555, 894644-895993, 896039-897463, 897595-899763, 900020- |
| | | | | 901015, 901102-905587, 905705-908984, 909154-912105, 912200-912850, |
| 56 | SUN1 | NM_001130965 | chr7 | 872239-878433, 878624-881581, 881768-882841, 882870-882976, 883158-891019, 891120-891585, 891681- |
| | | | | 892244, 892305-892445, 892590-893044, 893256-894555, 894644-895993, 896039-897463, 897595- |
| | | | | 899763, 900020-901015, 901102-905587, 905705-908984, 909154-912105, 912200-912850, |
| 56 | INTS1 | NM_001080453 | chr7 | 1510329-1510480, 1510592-1510767, 1510861-1511205, 1511278-1511907, 1512023-1512706, 1512821- |
| | | | | 1513200, 1513330-1513802, 1513997-1514339, 1514475-1515582, 1515708-1515863, 1516092- |
| | | | | 1516204, 1516294-1516428, 1516555-1517184, 1517310-1517387, 1517573-1518000, 1518143- |
| | | | | 1518360, 1518522-1519064, 1519321-1519951, 1520078-1520469, 1520570-1520976, 1521125- |
| | | | | 1522180, 1522344-1523376, 1523490-1523637, 1523807-1524732, 1524839-1524924, 1525106- |
| | | | | 1526259, 1526411-1526556, 1526738-1526954, 1527086-1527394, 1527594-1528978, 1529041- |
| | | | | 1529225, 1529319-1532644, 1532743-1533388, 1533548-1534450, 1534530-1535067, 1535190- |
| | | | | 1535790, 1535901-1536772, 1536951-1538046, 1538190-1538341, 1538489-1538609, 1538798- |
| | | | | 1538889, 1538997-1539107, 1539269-1539518, 1539658-1539804, 1540003-1542535, 1542828- |
| | | | | 1543537, 1543638-1543957, |
| 56 | PSMG3 | NM_001134340 | chr7 | 1607487-1608758, 1608997-1609643, |
| 56 | PSMG3 | NM_032302 | chr7 | 1607487-1608758, |
| 56 | MAD1L1 | NM_003550 | chr7 | 1855865-1937834, 1938027-1976321, 1976534-1997262, 1997355-2020086, 2020177-2041698, 2041757- |
| | | | | 2054135, 2054278-2108827, 2108974-2188785, 2188874-2252845, 2252909-2255518, 2255635- |
| | | | | 2255790, 2255923-2257595, 2257679-2258965, 2259092-2262208, 2262390-2265043, 2265186- |
| | | | | 2269617, 2269779-2270182, 2270400-2272505, |
| 56 | MAD1L1 | NM_001013836 | chr7 | 1855865-1937834, 1938027-1976321, 1976534-1997262, 1997355-2020086, 2020177-2041698, 2041757- |
| | | | | 2054135, 2054278-2108827, 2108974-2188785, 2188874-2252845, 2252909-2255518, 2255635- |
| | | | | 2255790, 2255923-2257595, 2257679-2258965, 2259092-2262208, 2262390-2265043, 2265186- |
| | | | | 2269617, 2269779-2270182, 2270363-2272505, |
| 56 | MAD1L1 | NM_001013837 | chr7 | 1855865-1937834, 1938027-1976321, 1976534-1997262, 1997355-2020086, 2020177-2041698, 2041757- |
| | | | | 2054135, 2054278-2108827, 2108974-2188785, 2188874-2252845, 2252909-2255518, 2255635- |
| | | | | 2255790, 2255923-2257595, 2257679-2258965, 2259092-2262208, 2262390-2265043, 2265186- |
| | | | | 2269617, 2269779-2270182, 2270360-2272505, |
| 56 | EIF3B | NM_003751 | chr7 | 2395056-2400343, 2400538-2402278, 2402400-2402703, 2402763-2403265, 2403396-2404005, 2404165- |
| | | | | 2405950, 2406084-2406158, 2406227-2406632, 2406681-2409105, 2409318-2411410, 2411485- |
| | | | | 2412306, 2412431-2414161, 2414242-2415022, 2415163-2416583, 2416711-2418322, 2418402- |
| | | | | 2418767, 2418878-2419027, 2419147-2419912, |
| 56 | EIF3B | NM_001037283 | chr7 | 2395056-2400343, 2400538-2402278, 2402400-2402703, 2402763-2403265, 2403396-2404005, 2404165- |
| | | | | 2405950, 2406084-2406158, 2406227-2406632, 2406681-2409105, 2409318-2411410, 2411485- |
| | | | | 2412306, 2412431-2414161, 2414242-2415022, 2415163-2416583, 2416711-2418322, 2418402- |
| | | | | 2418767, 2418878-2419027, 2419147-2419837, |
| 56 | RNF216L | NR_023384 | chr7 | 5013793-5015930, 5016068-5023300, 5023405-5024539, 5024707-5026196, 5026263-5028692, 5028809- |
| | | | | 5035104, 5035214-5036239, |
| 56 | RNF216L | NR_015449 | chr7 | 5013793-5015930, 5016068-5023300, 5023405-5024539, 5024707-5028692, 5028809-5036239, |
| 56 | RNF216L | NR_023385 | chr7 | 5013793-5015930, 5016068-5023300, 5023405-5024539, 5024707-5036239, |
| 56 | EIF2AK1 | NM_001134335 | chr7 | 6064433-6066357, 6066593-6068244, 6068329-6068547, 6068664-6077053, 6077156-6078189, 6078303- |
| | | | | 6080521, 6080851-6082562, 6082622-6084191, 6084293-6085700, 6085783-6086621, 6086723- |
| | | | | 6088436, 6088476-6089541, 6089677-6094175, 6094336-6098595, |
| 56 | EIF2AK1 | NM_014413 | chr7 | 6064433-6066357, 6066593-6068244, 6068329-6068547, 6068664-6077053, 6077156-6078189, 6078303- |
| | | | | 6080521, 6080851-6082562, 6082625-6084191, 6084293-6085700, 6085783-6086621, 6086723- |
| | | | | 6088436, 6088476-6089541, 6089677-6094175, 6094336-6098595, |
| 56 | KDELR2 | NM_001100603 | chr7 | 6502807-6509225, 6509386-6513813, 6513916-6523596, |
| 56 | KDELR2 | NM_006854 | chr7 | 6502807-6505700, 6505955-6509225, 6509386-6513813, 6513916-6523596, |
| 56 | RAPGEF5 | NM_012294 | chr7 | 22162140-22165220, 22165277-22170653, 22170720-22175654, 22175744-22176549, 22176602- |
| | | | | 22179641, 22179734-22184660, 22184841-22186513, 22186638-22190023, 22190123-22194071, 22194223- |
| | | | | 22196426, 22196507-22197471, 22197504-22200134, 22200234-22202013, 22202160-22206686, 22206767- |
| | | | | 22232983, 22233074-22233531, 22233652-22259483, 22259611-22270463, 22270539-22306581, 22306632- |
| | | | | 22330792, 22330861-22347956, 22348127-22349586, 22349710-22354987, 22355096-22357604, 22357657- |
| | | | | 22396447, |
| 56 | MGC87042 | NM_207342 | chr7 | 22459456-22532182, 22532349-22532941, 22533399-22534389, 22534506-22539731, |
| 56 | MGC87042 | NM_001164460 | chr7 | 22478375-22532182, 22532349-22532941, 22533456-22534389, 22534506-22539731, |
| 56 | TOMM7 | NM_019059 | chr7 | 22852805-22857617, 22857668-22862294, |
| 56 | FAM126A | NM_032581 | chr7 | 22985783-22999873, 23000035-23000852, 23000942-23004032, 23004151-23015827, 23015925- |
| | | | | 23016310, 23016428-23016958, 23017041-23017886, 23018068-23023561, 23023665-23030678, 23030759- |
| | | | | 23053542, |
| 56 | C7orf30 | NM_138446 | chr7 | 23339228-23340456, 23340637-23347485, 23347569-23348973, |
| 56 | NPY | NM_000905 | chr7 | 24323895-24324858, 24325048-24329116, 24329199-24331280, |
| 56 | MPP6 | NM_016447 | chr7 | 24613213-24659669, 24659840-24663283, 24663404-24681333, 24681488-24689229, 24689384- |
| | | | | 24690102, 24690332-24703207, 24703341-24705205, 24705307-24705638, 24705709-24708116, 24708280- |
| | | | | 24718748, 24718953-24720009, 24720140-24727055, |
| 56 | DFNA5 | NM_004403 | chr7 | 24738879-24742377, 24742453-24745801, 24745996-24747744, 24747874-24749841, 24750008- |
| | | | | 24756871, 24756994-24758664, 24758838-24784179, 24784374-24789181, 24789413-24797324, |
| 56 | DFNA5 | NM_001127453 | chr7 | 24738879-24742377, 24742453-24745801, 24745996-24747744, 24747874-24749841, 24750008- |
| | | | | 24756871, 24756994-24758664, 24758838-24784179, 24784374-24789181, 24789413-24797013, |
| 56 | DFNA5 | NM_001127454 | chr7 | 24738879-24742377, 24742453-24745801, 24745996-24747744, 24747874-24749841, 24750008- |
| | | | | 24756871, 24756994-24758664, 24758838-24784179, 24784374-24797324, |
| 56 | HNRNPA2B1 | NM_002137 | chr7 | 26231959-26232113, 26232198-26232869, 26232994-26233193, 26233315-26235465, 26235530- |
| | | | | 26236019, 26236102-26236175, 26236279-26236425, 26236638-26236933, 26237082-26237240, 26237353- |
| | | | | 26240190, |
| 56 | HNRNPA2B1 | NM_031243 | chr7 | 26231959-26232113, 26232198-26232869, 26232994-26233193, 26233315-26235465, 26235530- |
| | | | | 26236019, 26236102-26236175, 26236279-26236425, 26236638-26236933, 26237082-26237240, 26237353- |
| | | | | 26237449, 26237487-26240190, |
| 56 | CBX3 | NM_007276 | chr7 | 26241182-26242589, 26242643-26245986, 26246131-26248011, 26248176-26251280, 26251377-26251700, |
| 56 | CBX3 | NM_016587 | chr7 | 26241447-26242589, 26242643-26245986, 26246131-26248011, 26248176-26251280, 26251377-26251700, |
| 56 | HOXA11 | NM_005523 | chr7 | 27222648-27224053, |
| 56 | EVX1 | NM_001989 | chr7 | 27283077-27284665, 27284924-27285503, |
| 56 | HIBADH | NM_152740 | chr7 | 27565992-27570809, 27570968-27577958, 27578037-27582584, 27582720-27668988, 27669112- |
| | | | | 27671953, 27672065-27689090, 27689253-27702315, |
| 56 | TAX1BP1 | NM_006024 | chr7 | 27779889-27788135, 27788306-27797648, 27797753-27805451, 27805641-27809293, 27809454- |
| | | | | 27824780, 27824931-27825016, 27825109-27827035, 27827223-27831623, 27831850-27832683, 27832832- |
| | | | | 27833940, 27834066-27835733, 27835839-27839582, 27839710-27855966, 27856140-27856507, 27856658- |
| | | | | 27867355, 27867440-27868245, |
| 56 | TAX1BP1 | NM_001079864 | chr7 | 27779889-27788135, 27788306-27797648, 27797753-27805451, 27805641-27809293, 27809454- |
| | | | | 27824780, 27824931-27825016, 27825109-27827035, 27827223-27831623, 27831850-27832683, 27832832- |
| | | | | 27833940, 27834066-27835733, 27835839-27839582, 27839710-27855966, 27856014-27856507, 27856658- |
| | | | | 27867355, 27867440-27868245, |
| 56 | PLEKHA8 | NM_001197026 | chr7 | 30068419-30084699, 30084818-30085824, 30085982-30087446, 30087573-30088838, 30088999- |
| | | | | 30090048, 30090091-30092323, 30092483-30094323, 30094482-30094871, 30094959-30100498, 30100559- |
| | | | | 30101511, 30101644-30102286, 30102359-30113685, 30113749-30118204, |
| 56 | PLEKHA8 | NM_032639 | chr7 | 30068419-30084699, 30084818-30085824, 30085982-30087446, 30087573-30088838, 30088999- |
| | | | | 30090048, 30090091-30092323, 30092483-30094323, 30094482-30094871, 30094959-30100498, 30100559- |
| | | | | 30101511, 30101644-30102286, 30102359-30129767, |
| 56 | PLEKHA8 | NM_001197027 | chr7 | 30068419-30084699, 30084818-30085824, 30085982-30087446, 30087573-30088838, 30088999- |
| | | | | 30090048, 30090091-30092323, 30092483-30094323, 30094482-30094871, 30094959-30100498, 30100559- |
| | | | | 30101511, 30101644-30102286, 30102359-30113685, 30113749-30157584, |
| 56 | ZNRF2 | NM_147128 | chr7 | 30325443-30363256, 30363354-30395342, 30395450-30401991, 30402073-30405649, |
| 56 | NOD1 | NM_006092 | chr7 | 30465327-30468988, 30469074-30472710, 30472796-30475612, 30475698-30477187, 30477273- |
| | | | | 30485755, 30485841-30486581, 30486667-30487912, 30487998-30490830, 30492657-30494751, 30494928- |
| | | | | 30496335, 30496659-30498766, 30498857-30499515, 30499658-30518220, |
| 56 | GGCT | NR_037669 | chr7 | 30536852-30538417, 30538555-30544183, |
| 56 | GGCT | NM_001199815 | chr7 | 30536852-30537355, 30537513-30540150, 30540298-30544183, |
| 56 | GGCT | NM_001199817 | chr7 | 30536852-30544183, |
| 56 | GGCT | NM_001199816 | chr7 | 30536852-30540150, 30540298-30544183, |
| 56 | GGCT | NM_024051 | chr7 | 30536852-30538417, 30538555-30540150, 30540298-30544183, |
| 56 | GARS | NM_002047 | chr7 | 30634760-30638410, 30638514-30639561, 30639666-30640673, 30640817-30642648, 30642739- |
| | | | | 30643110, 30643189-30649199, 30649347-30651710, 30651862-30655510, 30655675-30656728, 30656895- |
| | | | | 30661007, 30661117-30661931, 30662079-30665848, 30665936-30668174, 30668286-30671062, 30671158- |
| | | | | 30671861, 30672054-30673349, |
| 56 | SEPT7 | NM_001011553 | chr7 | 35840883-35872406, 35872511-35903160, 35903269-35912274, 35912377-35913249, 35913386- |
| | | | | 35919431, 35919551-35922092, 35922187-35923499, 35923598-35925436, 35925490-35930276, 35930404- |
| | | | | 35937856, 35937994-35942684, 35942826-35943862, |
| 56 | SEPT7 | NM_001788 | chr7 | 35840884-35872406, 35872511-35903160, 35903269-35912274, 35912377-35913249, 35913386- |
| | | | | 35919431, 35919551-35922092, 35922187-35923499, 35923598-35925436, 35925490-35930276, 35930404- |
| | | | | 35937856, 35937994-35942684, 35942826-35943862, |
| 56 | ANLN | NM_018685 | chr7 | 36429654-36435873, 36436029-36438686, 36439003-36445788, 36446176-36447341, 36447566- |
| | | | | 36450121, 36450314-36450666, 36450776-36455365, 36455494-36456687, 36456800-36458851, 36459089- |
| | | | | 36459776, 36459924-36460204, 36460354-36461464, 36461602-36462240, 36462419-36463424, 36463553- |
| | | | | 36464152, 36464203-36464293, 36464352-36465309, 36465350-36465622, 36465646-36466523, 36466638- |
| | | | | 36478811, 36478900-36483362, 36483472-36489272, 36489447-36492084, |
| 56 | VPS41 | NM_080631 | chr7 | 38765927-38766507, 38766589-38768140, 38768187-38768290, 38768392-38781583, 38781722- |
| | | | | 38783000, 38783143-38785157, 38785214-38791774, 38791914-38794300, 38794353-38794493, 38794537- |
| | | | | 38796436, 38796583-38797952, 38798082-38803053, 38803148-38805178, 38805262-38807135, 38807199- |
| | | | | 38810796, 38810855-38812120, 38812238-38813713, 38813845-38816277, 38816377-38829399, 38829468- |
| | | | | 38835063, 38835212-38836343, 38836465-38857415, 38857483-38860801, 38860866-38902143, 38902223- |
| | | | | 38908744, 38908854-38937689, 38937730-38948752, |
| 56 | VPS41 | NM_014396 | chr7 | 38765927-38766507, 38766589-38768140, 38768187-38768290, 38768392-38781583, 38781722- |
| | | | | 38783000, 38783143-38785157, 38785214-38791774, 38791914-38794300, 38794353-38794493, 38794537- |
| | | | | 38796436, 38796583-38797952, 38798082-38803053, 38803148-38805178, 38805262-38807135, 38807199- |
| | | | | 38810796, 38810855-38812120, 38812238-38813713, 38813845-38816277, 38816377-38829399, 38829468- |
| | | | | 38835063, 38835212-38836343, 38836465-38857415, 38857483-38860801, 38860866-38869852, 38869929- |
| | | | | 38902143, 38902223-38908744, 38908854-38937689, 38937730-38948752, |
| 56 | CDK13 | NM_003718 | chr7 | 39991452-40027196, 40027858-40037091, 40037264-40038958, 40039100-40041458, 40041631- |
| | | | | 40085433, 40085625-40087418, 40087477-40102423, 40102527-40102620, 40102700-40117602, 40117721- |
| | | | | 40118317, 40118451-40127723, 40127931-40132382, 40132837-40133727, |
| 56 | CDK13 | NM_031267 | chr7 | 39991452-40027196, 40027858-40037091, 40037264-40038958, 40039100-40041458, 40041631- |
| | | | | 40085433, 40085625-40087418, 40087477-40102423, 40102527-40102620, 40102700-40117602, 40117721- |
| | | | | 40118317, 40118451-40127723, 40127931-40132562, 40132837-40133727, |
| 56 | PPIA | NM_021130 | chr7 | 44836393-44838844, 44838877-44838989, 44839080-44839299, 44839474-44840884, |
| 56 | TBRG4 | NM_004749 | chr7 | 45140026-45140855, 45140972-45141100, 45141214-45141422, 45141670-45141944, 45142091- |
| | | | | 45142930, 45143043-45143696, 45143856-45144135, 45144309-45145038, 45145364-45148424, 45148887- |
| | | | | 45151240, |
| 56 | TBRG4 | NM_199122 | chr7 | 45140026-45140855, 45140972-45141100, 45141214-45141422, 45141670-45141944, 45142091- |
| | | | | 45142930, 45143043-45145038, 45145364-45148424, 45148887-45151261, |
| 56 | TBRG4 | NM_030900 | chr7 | 45140026-45140855, 45140972-45141100, 45141214-45141422, 45141670-45141944, 45142091- |
| | | | | 45142930, 45143043-45145038, 45145364-45148424, 45148887-45151240, |
| 56 | IGFBP3 | NM_000598 | chr7 | 45953434-45954402, 45954545-45956145, 45956267-45956810, 45957039-45960335, |
| 56 | IGFBP3 | NM_001013398 | chr7 | 45953434-45954402, 45954545-45956145, 45956267-45956810, 45957039-45960317, |
| 56 | ZNF716 | NM_001159279 | chr7 | 57510034-57522164, 57522293-57522777, 57522875-57528428, |
| 57 | PHIP | NM_017934 | chr6 | 79651048-79655015, 79655215-79655716, 79655978-79656426, 79656592-79657338, 79657493- |
| | | | | 79664529, 79664681-79664947, 79665000-79665328, 79665400-79668190, 79668318-79671405, 79671528- |
| | | | | 79672812, 79672970-79675418, 79675482-79675660, 79675774-79679550, 79679635-79679764, 79679891- |
| | | | | 79680496, 79680606-79688307, 79688429-79692601, 79692835-79695067, 79695146-79697924, 79698067- |
| | | | | 79700583, 79700703-79707129, 79707315-79707969, 79708109-79711614, 79711842-79713445, 79713576- |
| | | | | 79724797, 79724934-79725345, 79725501-79726259, 79726360-79727061, 79727104-79727198, 79727301- |
| | | | | 79728804, 79728877-79735234, 79735337-79735658, 79735882-79752558, 79752721-79770193, 79770294- |
| | | | | 79770383, 79770536-79787163, 79787225-79787415, 79787447-79787570, 79787631-79787744, |
| 57 | TTK | NM_001166691 | chr6 | 80714431-80715545, 80715700-80717524, 80717749-80718101, 80718210-80720529, 80720675- |
| | | | | 80721149, 80721266-80721444, 80721519-80721601, 80721698-80722994, 80723084-80724178, 80724304- |
| | | | | 80732039, 80732190-80736096, 80736232-80737600, 80737729-80741182, 80741277-80744700, 80744860- |
| | | | | 80744981, 80745135-80746190, 80746317-80747682, 80747765-80749411, 80749590-80749911, 80749998- |
| | | | | 80750321, 80750421-80751834, |
| 57 | TTK | NM_003318 | chr6 | 80714431-80715557, 80715700-80717524, 80717749-80718101, 80718210-80720529, 80720675- |
| | | | | 80721149, 80721266-80721444, 80721519-80721601, 80721698-80722994, 80723084-80724178, 80724304- |
| | | | | 80732039, 80732190-80736093, 80736232-80737600, 80737729-80741182, 80741277-80744700, 80744860- |
| | | | | 80744981, 80745135-80746190, 80746317-80747682, 80747765-80749411, 80749590-80749911, 80749998- |
| | | | | 80750321, 80750421-80751834, |
| 57 | ME1 | NM_002395 | chr6 | 83921814-83926215, 83926316-83933477, 83933653-83937052, 83937197-83938543, 83938651- |
| | | | | 83947434, 83947550-83949256, 83949356-83963346, 83963458-84025027, 84025133-84055890, 84056054- |
| | | | | 84061781, 84061859-84108084, 84108236-84117485, 84117621-84140594, |
| 57 | RIPPLY2 | NM_001009994 | chr6 | 84563231-84563412, 84563493-84563814, 84563881-84566959, |
| 57 | PRDM1 | NM_182907 | chr6 | 106546970-106547173, 106547428-106552698, 106553809-106554244, 106554375-106554784, |
| 57 | PRDM1 | NM_001198 | chr6 | 106534471-106536074, 106536325-106543488, 106543610-106547173, 106547428- |
| | | | | 106552698, 106553809-106554244, 106554375-106554784, |
| 57 | ATG5 | NM_004849 | chr6 | 106634552-106649845, 106649965-106696023, 106696120-106727534, 106727699- |
| | | | | 106740901, 106740982-106756237, 106756367-106763974, 106764142-106773399, |
| 57 | PDSS2 | NM_020381 | chr6 | 107475982-107515024, 107515059-107531641, 107531775-107533311, 107533487- |
| | | | | 107566750, 107566824-107595231, 107595432-107655400, 107655537-107780192, |
| 58 | TMEM221 | NM_001190844 | chr19 | 17547737-17556006, 17556094-17558950, |
| 58 | COMP | NM_000095 | chr19 | 18893772-18893862, 18894004-18894999, 18895174-18895704, 18895903-18896306, 18896357- |
| | | | | 18896481, 18896662-18896773, 18896957-18897047, 18897102-18897340, 18897461-18898298, 18898460- |
| | | | | 18899019, 18899129-18899217, 18899324-18899399, 18899560-18899646, 18899723-18899967, 18900107- |
| | | | | 18900749, 18900924-18901369, 18901423-18901657, 18901745-18901998, |
| 58 | ZNF626 | NM_145297 | chr19 | 20828255-20828488, 20828586-20829083, 20829212-20844248, |
| 58 | ZNF626 | NM_001076675 | chr19 | 20808457-20828488, 20828586-20829083, 20829212-20844248, |
| 58 | ZNF85 | NM_003429 | chr19 | 21106210-21116828, 21116957-21117753, 21117854-21131548, |
| 58 | ZNF85 | NR_034060 | chr19 | 21106210-21116828, 21116957-21117753, 21117854-21125605, |
| 58 | ZNF738 | NR_027130 | chr19 | 21541946-21544566, 21544661-21558038, 21558167-21558669, 21558767-21560189, |
| 58 | ZNF429 | NM_001001415 | chr19 | 21688577-21712458, 21712587-21713389, 21713487-21719080, |
| 59 | FAM115A | NM_014719 | chr7 | 143551236-143554191, 143554444-143555915, 143556255-143557299, 143557541- |
| | | | | 143558186, 143558402-143559507, 143559607-143559893, 143560890-143573080, 143573716- |
| | | | | 143599052, |
| 59 | ZNF425 | NM_001001661 | chr7 | 148802659-148809227, 148809388-148815312, 148815441-148823286, |
| 59 | ZNF862 | NM_001099220 | chr7 | 149535725-149541712, 149541826-149543238, 149543345-149544822, 149545522- |
| | | | | 149547248, 149547428-149556509, 149556616-149557470, 149559584-149561196, |
| 59 | LOC401431 | NR_027040 | chr7 | 149566948-149567329, 149567441-149568729, 149568886-149570311, |
| 59 | PRKAG2 | NM_001040633 | chr7 | 151254319-151257608, 151257704-151261162, 151261311-151262430, 151262470- |
| | | | | 151262804, 151262972-151265800, 151265929-151267255, 151267312-151269748, 151269796- |
| | | | | 151271975, 151272036-151273455, 151273539-151292429, 151292541-151329153, 151329225- |
| | | | | 151372504, 151372724-151478236, 151478518-151483554, 151483628-151511500, |
| 59 | PRKAG2 | NM_024429 | chr7 | 151254319-151257608, 151257704-151261162, 151261311-151262430, 151262470- |
| | | | | 151262804, 151262972-151265800, 151265929-151267255, 151267312-151269748, 151269796- |
| | | | | 151271975, 151272036-151273455, 151273539-151292429, 151292541-151329153, |
| 59 | PRKAG2 | NM_016203 | chr7 | 151254319-151257608, 151257704-151261162, 151261311-151262430, 151262470- |
| | | | | 151262804, 151262972-151265800, 151265929-151267255, 151267312-151269748, 151269796- |
| | | | | 151271975, 151272036-151273455, 151273539-151292429, 151292541-151329153, 151329225- |
| | | | | 151372504, 151372724-151478236, 151478518-151483554, 151483628-151573590, |
| 59 | LOC100132707 | NR_024477 | chr7 | 154720478-154720664, 154720735-154735735, 154736331-154738068, 154738204-154738348, |
| 59 | LOC100132707 | NR_024476 | chr7 | 154720478-154720664, 154720735-154735735, 154736331-154738068, 154738204- |
| | | | | 154738301, 154738502-154739401, 154739484-154739606, 154739801-154741006, |
| 60 | C15orf23 | NM_001142762 | chr15 | 40675246-40675427, 40675524-40678561, 40678696-40679358, 40679408-40681705, 40681813- |
| | | | | 40682035, 40682131-40684148, 40684225-40685668, |
| 60 | C15orf23 | NM_033286 | chr15 | 40675246-40675427, 40675524-40678561, 40678696-40679358, 40679408-40681705, 40681813- |
| | | | | 40682035, 40682131-40683692, 40683756-40684148, 40684225-40685668, |
| 60 | C15orf23 | NM_001142761 | chr15 | 40675246-40675427, 40675524-40678561, 40678696-40679358, 40679408-40681705, 40681813- |
| | | | | 40682035, 40682131-40683692, 40683756-40684148, 40684225-40685392, |
| 60 | CASC5 | NM_170589 | chr15 | 40886591-40895111, 40895165-40897306, 40897348-40898589, 40898651-40901043, 40901107- |
| | | | | 40902441, 40902496-40902995, 40903075-40903674, 40903710-40907537, 40907577-40911155, 40911210- |
| | | | | 40912836, 40917839-40920266, 40920407-40920807, 40920877-40921469, 40921570-40933108, 40933226- |
| | | | | 40937193, 40937286-40937852, 40937971-40939183, 40939273-40942497, 40942577-40942740, 40942782- |
| | | | | 40943667, 40943771-40944201, 40944304-40947105, 40947176-40949238, 40949350-40949551, 40949672- |
| | | | | 40951535, 40951660-40954270, |
| 60 | CASC5 | NM_144508 | chr15 | 40886591-40895111, 40895165-40897306, 40897348-40898589, 40898651-40901043, 40901107- |
| | | | | 40902441, 40902496-40903674, 40903710-40907537, 40907577-40911155, 40911210-40912836, 40917839- |
| | | | | 40920266, 40920407-40920807, 40920877-40921469, 40921570-40933108, 40933226-40937193, 40937286- |
| | | | | 40937852, 40937971-40939183, 40939273-40942497, 40942577-40942740, 40942782-40943667, 40943771- |
| | | | | 40944201, 40944304-40947105, 40947176-40949238, 40949350-40949551, 40949672-40951535, 40951660- |
| | | | | 40954270, |
| 60 | RAD51 | NM_002875 | chr15 | 40987624-40990953, 40991044-40993260, 40993400-40998373, 40998493-41001221, 41001315- |
| | | | | 41011001, 41011098-41020907, 41021023-41021701, 41021833-41022049, 41022173-41023251, |
| 60 | RAD51 | NM_133487 | chr15 | 40987624-40990953, 40991044-40993260, 40993400-40994002, 40994125-41001221, 41001315- |
| | | | | 41011001, 41011098-41020907, 41021023-41021701, 41021833-41022049, 41022173-41023251, |
| 60 | RAD51 | NM_001164270 | chr15 | 40987624-40990953, 40991044-40993260, 40993400-40998373, 40998493-41001221, 41001315- |
| | | | | 41011001, 41011098-41020907, 41021023-41021701, 41021833-41023251, |
| 60 | RAD51 | NM_001164269 | chr15 | 40987520-40990953, 40991044-40993260, 40993400-40994002, 40994125-41001221, 41001315- |
| | | | | 41011001, 41011098-41020907, 41021023-41021701, 41021833-41022049, 41022173-41023251, |
| 60 | RHOV | NM_133639 | chr15 | 41165700-41165880, 41165941-41166128, |
| 60 | INO80 | NM_017553 | chr15 | 41272639-41275058, 41275276-41275958, 41276120-41276393, 41276551-41277534, 41277625- |
| | | | | 41279287, 41279392-41280012, 41280173-41289725, 41289800-41297789, 41297886-41308284, 41308414- |
| | | | | 41313096, 41313324-41319792, 41319935-41337100, 41337274-41339604, 41339700-41340408, 41340475- |
| | | | | 41341483, 41341619-41342131, 41342301-41346125, 41346213-41347443, 41347563-41348818, 41348905- |
| | | | | 41350835, 41350980-41352063, 41352125-41361766, 41361864-41362663, 41362746-41364045, 41364257- |
| | | | | 41365624, 41365694-41366566, 41366764-41371897, 41372103-41373216, 41373272-41377565, 41377782- |
| | | | | 41379758, 41379881-41384223, 41384381-41387797, 41387867-41387955, 41388127-41388364, 41388552- |
| | | | | 41408169, |
| 60 | OIP5 | NM_007280 | chr15 | 41602048-41605469, 41605553-41611854, 41611979-41624111, 41624180-41624436, |
| 60 | NUSAP1 | NM_001129897 | chr15 | 41625249-41634582, 41634653-41641294, 41641440-41643184, 41643328-41648235, 41648339- |
| | | | | 41650345, 41650457-41657598, 41657788-41663723, 41663883-41669392, 41669503-41672289, |
| 60 | NUSAP1 | NM_018454 | chr15 | 41625249-41634582, 41634653-41641294, 41641440-41643184, 41643328-41648235, 41648339- |
| | | | | 41650345, 41650457-41657601, 41657788-41663723, 41663883-41667908, 41668027-41669392, 41669503- |
| | | | | 41672289, |
| 60 | NUSAP1 | NM_016359 | chr15 | 41625249-41634582, 41634653-41641294, 41641440-41643184, 41643328-41648235, 41648339- |
| | | | | 41650345, 41650457-41657598, 41657788-41663723, 41663883-41667908, 41668027-41669392, 41669503- |
| | | | | 41672289, |
| 60 | UBR1 | NM_174916 | chr15 | 43237669-43242458, 43242562-43244474, 43244647-43250209, 43250289-43252841, 43252900- |
| | | | | 43256131, 43256242-43258349, 43258485-43262716, 43262806-43268913, 43269066-43270076, 43270146- |
| | | | | 43276093, 43276192-43277083, 43277141-43281015, 43281166-43282226, 43282319-43290364, 43290464- |
| | | | | 43294751, 43294903-43296033, 43296129-43299275, 43299483-43307884, 43308068-43309291, 43309380- |
| | | | | 43313471, 43313574-43314898, 43315000-43317025, 43317182-43317577, 43317628-43318757, 43318862- |
| | | | | 43319972, 43320027-43322140, 43322267-43324764, 43324830-43328374, 43328478-43328724, 43328792- |
| | | | | 43329969, 43330082-43330367, 43330431-43335411, 43335594-43339357, 43339488-43340588, 43340690- |
| | | | | 43346938, 43347098-43348540, 43348641-43350537, 43350628-43351281, 43351391-43351898, 43352024- |
| | | | | 43352248, 43352313-43360094, 43360235-43362991, 43363124-43367175, 43367288-43374834, 43374915- |
| | | | | 43378180, 43378439-43398138, |
| 60 | ELL3 | NM_025165 | chr15 | 44065425-44065500, 44065547-44066378, 44066552-44066672, 44066717-44066792, 44066972- |
| | | | | 44067502, 44067580-44067721, 44067809-44067918, 44068122-44068235, 44068350-44068705, 44068743- |
| | | | | 44068966, |
| 61 | ACER2 | NM_001010887 | chr9 | 19409191-19423858, 19423975-19424696, 19424840-19434943, 19435083-19446277, 19446417-19450446, |
| 61 | IFT74 | NM_001099222 | chr9 | 26947462-26961945, 26962086-26978124, 26978262-26980567, 26980618-26984253, 26984354- |
| | | | | 26984495, 26984558-26988665, 26988727-26990130, 26990194-27009016, 27009157-27011902, 27011967- |
| | | | | 27016903, 27017049-27018643, 27018686-27029021, 27029103-27044738, 27044794-27047270, 27047370- |
| | | | | 27048144, 27048273-27055605, 27055771-27056330, 27056458-27060587, 27060650-27062614, |
| 61 | IFT74 | NM_025103 | chr9 | 26956515-26961945, 26962086-26978124, 26978262-26980567, 26980618-26984253, 26984354- |
| | | | | 26984495, 26984558-26988665, 26988727-26990130, 26990194-27009016, 27009157-27011902, 27011967- |
| | | | | 27016903, 27017049-27018643, 27018686-27029021, 27029103-27044738, 27044794-27047270, 27047370- |
| | | | | 27048144, 27048273-27055605, 27055771-27056330, 27056458-27060587, 27060650-27062614, |
| 61 | IFT74 | NM_001099224 | chr9 | 26956515-26961945, 26962086-26978124, 26978262-26980567, 26980618-26984253, 26984354- |
| | | | | 26984495, 26984558-26988665, 26988727-26990130, 26990194-27009016, 27009157-27011902, 27011967- |
| | | | | 27016903, 27017049-27018643, 27018686-27029021, 27029103-27036446, |
| 61 | IFT74 | NM_001099223 | chr9 | 26947145-26961945, 26962086-26978124, 26978262-26980567, 26980618-26984253, 26984354- |
| | | | | 26984495, 26984558-26988665, 26988727-26990130, 26990194-27009016, 27009157-27011902, 27011967- |
| | | | | 27016903, 27017049-27018643, 27018686-27029021, 27029103-27044738, 27044794-27047270, 27047370- |
| | | | | 27048144, 27048273-27055605, 27055771-27056330, 27056458-27060587, 27060650-27062614, |
| 61 | TOPORS | NM_005802 | chr9 | 32544325-32550770, 32550967-32552430, |
| 61 | TOPORS | NM_001195622 | chr9 | 32544325-32552430, |
| 61 | NDUFB6 | NM_001199987 | chr9 | 32553943-32558906, 32558953-32572877, |
| 61 | NDUFB6 | NM_002493 | chr9 | 32553943-32558906, 32558953-32570956, 32571051-32572877, |
| 61 | NDUFB6 | NM_182739 | chr9 | 32553943-32570956, 32571051-32572877, |
| 61 | DNAJA1 | NM_001539 | chr9 | 33025382-33026471, 33026615-33026809, 33026989-33029881, 33029988-33030436, 33030666- |
| | | | | 33034212, 33034329-33036570, 33036688-33037011, 33037114-33038681, |
| 61 | DCTN3 | NM_007234 | chr9 | 34613869-34614037, 34614099-34614705, 34614766-34616025, 34616111-34617880, 34617969- |
| | | | | 34618671, 34618758-34620364, |
| 61 | DCTN3 | NM_024348 | chr9 | 34613869-34614037, 34614196-34614705, 34614766-34616025, 34616111-34617880, 34617969- |
| | | | | 34618671, 34618758-34620364, |
| 61 | RUSC2 | NM_001135999 | chr9 | 35539106-35546425, 35548533-35555055, 35555699-35555947, 35556135-35556303, 35556446- |
| | | | | 35557909, 35557988-35558192, 35558369-35558457, 35558565-35559221, 35559270-35560024, 35560849- |
| | | | | 35560955, 35561095-35561176, |
| 61 | RUSC2 | NM_014806 | chr9 | 35490170-35546425, 35548533-35555055, 35555699-35555947, 35556135-35556303, 35556446- |
| | | | | 35557909, 35557988-35558192, 35558369-35558457, 35558565-35559221, 35559270-35560024, 35560849- |
| | | | | 35560955, 35561095-35561176, |
| 61 | CREB3 | NM_006368 | chr9 | 35732899-35732991, 35733141-35733210, 35733280-35733391, 35733483-35735104, 35735213- |
| | | | | 35735301, 35735372-35736043, 35736130-35736222, 35736309-35736387, |
| 61 | TMEM8B | NM_001042590 | chr9 | 35829953-35834456, 35834648-35835006, 35835216-35841129, 35841265-35841521, 35841792- |
| | | | | 35842387, 35842715-35845970, 35846066-35846253, 35846379-35846464, 35846609-35846812, 35846993- |
| | | | | 35852822, 35852971-35853136, 35853255-35853500, |
| 61 | TMEM8B | NM_001042589 | chr9 | 35829317-35829872, 35829953-35834456, 35834648-35835006, 35835216-35841129, 35841265- |
| | | | | 35841521, 35841792-35842387, 35842715-35845970, 35846066-35846253, 35846379-35846464, 35846609- |
| | | | | 35846812, 35846993-35852822, 35852971-35853136, 35853255-35853500, |
| 61 | TMEM8B | NM_016446 | chr9 | 35829389-35829872, 35829953-35834456, 35834648-35835006, 35835216-35841129, 35841265- |
| | | | | 35841521, 35841792-35842387, 35842715-35845970, 35846066-35846253, 35846379-35846464, 35846609- |
| | | | | 35846812, |
| 62 | MRPL54 | NM_172251 | chr19 | 3762817-3765162, 3765330-3767257, |
| 62 | PIAS4 | NM_015897 | chr19 | 4007786-4012919, 4013348-4024032, 4024119-4028142, 4028186-4028506, 4028599-4028716, 4028847- |
| | | | | 4028927, 4029035-4033096, 4033172-4033416, 4033579-4037370, 4037503-4037612, |
| 62 | MAP2K2 | NM_030662 | chr19 | 4090707-4094449, 4094497-4095384, 4095448-4097275, 4097342-4099197, 4099413-4101015, 4101142- |
| | | | | 4101225, 4101279-4102372, 4102452-4110505, 4110654-4117415, 4117628-4123779, |
| 62 | SIRT6 | NM_016539 | chr19 | 4174944-4175023, 4175149-4175675, 4175758-4175837, 4175935-4177074, 4177136-4179099, 4179284- |
| | | | | 4180777, 4180907-4182469, |
| 62 | SIRT6 | NM_001193285 | chr19 | 4174944-4175023, 4175149-4175837, 4175935-4177074, 4177136-4179099, 4179284-4180777, 4180907- |
| | | | | 4182469, |
| 62 | ANKRD24 | NM_133475 | chr19 | 4183475-4186269, 4186459-4199678, 4199767-4199870, 4200003-4200078, 4200169-4202021, 4202088- |
| | | | | 4202864, 4202924-4207237, 4207310-4207496, 4207605-4207776, 4207966-4208759, 4208799- |
| | | | | 4210053, 4210136-4210260, 4210370-4212470, 4212511-4212595, 4212696-4215973, 4216048- |
| | | | | 4216279, 4216400-4216545, 4218161-4219586, 4219756-4222665, 4222793-4224122, 4224190-4224423, |
| 62 | HDGFRP2 | NM_001001520 | chr19 | 4472420-4475270, 4475349-4475440, 4475581-4488671, 4488874-4491561, 4491680-4491759, 4491833- |
| | | | | 4493698, 4493860-4493977, 4494055-4494161, 4494473-4496297, 4496403-4497953, 4498029- |
| | | | | 4498301, 4498374-4498809, 4498913-4499486, 4499635-4499648, 4499717-4501201, 4501330-4501921, |
| 62 | HDGFRP2 | NM_032631 | chr19 | 4472420-4475270, 4475349-4475440, 4475581-4488671, 4488874-4491561, 4491680-4491759, 4491833- |
| | | | | 4493698, 4493860-4493977, 4494055-4494161, 4494473-4496297, 4496403-4497953, 4498029- |
| | | | | 4498301, 4498374-4498809, 4498913-4499486, 4499635-4499648, 4499717-4501201, 4501330-4501924, |
| 62 | KDM4B | NM_015015 | chr19 | 4969242-5016266, 5016351-5032875, 5033043-5039845, 5040023-5041146, 5041263-5047485, 5047681- |
| | | | | 5071019, 5071071-5077376, 5077482-5082376, 5082516-5110631, 5110830-5119662, 5119864- |
| | | | | 5131085, 5131557-5131896, 5132019-5133892, 5134073-5135348, 5135573-5137271, 5137350- |
| | | | | 5137630, 5137688-5137971, 5138082-5143976, 5144164-5144257, 5144424-5144792, 5144914- |
| | | | | 5150367, 5150462-5151344, |
| 62 | ACSBG2 | NM_030924 | chr19 | 6135921-6141522, 6141622-6147455, 6147687-6151716, 6151807-6156440, 6156563-6161224, 6161307- |
| | | | | 6165875, 6166027-6177238, 6177408-6182760, 6182944-6183048, 6183284-6185445, 6185665- |
| | | | | 6187292, 6187434-6187608, 6187857-6190593, 6190704-6192677, |
| 62 | MLLT1 | NM_005934 | chr19 | 6213182-6213346, 6213420-6213735, 6213809-6213948, 6214050-6216414, 6216525-6217963, 6218053- |
| | | | | 6222130, 6222696-6226986, 6227114-6230579, 6230725-6262237, 6262322-6270588, 6270771-6279782, |
| 62 | GTF2F1 | NM_002096 | chr19 | 6380497-6380582, 6380702-6380913, 6381054-6381131, 6381207-6381368, 6381490-6381563, 6381627- |
| | | | | 6381706, 6381862-6383320, 6383507-6387398, 6387571-6389453, 6389649-6391911, 6391986- |
| | | | | 6392866, 6392915-6392993, |
| 62 | TRIP10 | NM_004240 | chr19 | 6739797-6741019, 6741137-6741234, 6741293-6742976, 6743126-6743203, 6743268-6743503, 6743610- |
| | | | | 6743717, 6743848-6744563, 6744712-6744809, 6745006-6746461, 6746573-6749943, 6750078- |
| | | | | 6750301, 6750443-6750521, 6750645-6751072, |
| 62 | ZNF358 | NM_018083 | chr19 | 7581136-7584089, |
| 62 | XAB2 | NM_020196 | chr19 | 7684534-7684631, 7684743-7684830, 7684962-7685159, 7685333-7685431, 7685556-7685730, 7685923- |
| | | | | 7686019, 7686184-7687215, 7687331-7687414, 7687548-7687646, 7687780-7688054, 7688176- |
| | | | | 7688615, 7688769-7689185, 7689332-7690764, 7690931-7691020, 7691157-7692127, 7692327- |
| | | | | 7692612, 7692738-7693034, 7693185-7694361, |
| 62 | MAP2K7 | NM_145185 | chr19 | 7968954-7974638, 7974782-7974946, 7975015-7975143, 7975259-7975336, 7975458-7975579, 7975689- |
| | | | | 7975863, 7976045-7976133, 7976216-7976319, 7976464-7977032, 7977080-7977180, |
| 63 | MGC3771 | NR_024166 | chr16 | 3160956-3164794, 3164889-3164967, |
| 63 | MGC3771 | NR_024167 | chr16 | 3160956-3164794, 3164884-3164967, |
| 63 | ZNF263 | NM_005741 | chr16 | 3334206-3335057, 3335240-3335679, 3335755-3336021, 3336150-3338452, 3338571-3339391, |
| 63 | TIGD7 | NM_033208 | chr16 | 3352010-3355210, |
| 63 | ZNF434 | NM_017810 | chr16 | 3433712-3434457, 3434942-3440008, 3440184-3440421, 3440518-3447190, 3447788-3450972, |
| 63 | ZNF597 | NM_152457 | chr16 | 3487539-3490805, 3490934-3493119, 3493207-3493358, |
| 63 | NAT15 | NM_001083601 | chr16 | 3493838-3498469, 3498541-3526220, 3526338-3529469, 3529601-3532500, 3532599-3533361, 3533598- |
| | | | | 3534697, 3535062-3535465, |
| 63 | NAT15 | NM_024845 | chr16 | 3508182-3526220, 3526338-3529469, 3529601-3532500, 3532599-3533361, 3533598-3534697, 3535084- |
| | | | | 3535465, |
| 63 | NAT15 | NM_001083600 | chr16 | 3508182-3526220, 3526338-3529469, 3529601-3532500, 3532599-3533361, 3533598-3534697, 3535062- |
| | | | | 3535465, |
| 63 | CREBBP | NM_004380 | chr16 | 3779876-3781191, 3781475-3781775, 3781939-3786035, 3786205-3786649, 3786817-3788558, 3788674- |
| | | | | 3789577, 3789726-3790398, 3790551-3794893, 3794963-3795276, 3795356-3799626, 3799685- |
| | | | | 3801725, 3801808-3807287, 3807378-3807808, 3808050-3808853, 3808974-3817719, 3817911- |
| | | | | 3819173, 3819355-3820569, 3820988-3823750, 3823932-3824568, 3824695-3827612, 3827659- |
| | | | | 3828010, 3828184-3828699, 3828819-3830731, 3830880-3831203, 3831308-3832683, 3832928- |
| | | | | 3841980, 3842096-3843385, 3843628-3860602, 3860781-3900296, 3901011-3929831, |
| 63 | CREBBP | NM_001079846 | chr16 | 3779876-3781191, 3781475-3781775, 3781939-3786035, 3786205-3786649, 3786817-3788558, 3788674- |
| | | | | 3789577, 3789726-3790398, 3790551-3794893, 3794963-3795276, 3795356-3799626, 3799685- |
| | | | | 3801725, 3801808-3807287, 3807378-3807808, 3808050-3808853, 3808974-3817719, 3817911- |
| | | | | 3819173, 3819355-3820569, 3820988-3823750, 3823932-3824568, 3824695-3827612, 3827659- |
| | | | | 3828010, 3828184-3828699, 3828819-3830731, 3830880-3831203, 3831308-3832683, 3832928- |
| | | | | 3843385, 3843628-3860602, 3860781-3900296, 3901011-3929831, |
| 63 | C16orf5 | NM_013399 | chr16 | 4562685-4562790, 4563066-4563695, 4563853-4564045, 4564146-4564574, 4564666-4588701, |
| 63 | C16orf5 | NM_001199054 | chr16 | 4562685-4562790, 4563066-4563695, 4563853-4564045, 4564146-4564574, 4564666-4588324, |
| 63 | C16orf5 | NM_001199055 | chr16 | 4562685-4562790, 4562949-4563695, 4563853-4564045, 4564146-4564574, 4564666-4588701, |
| 63 | C16orf5 | NM_001199056 | chr16 | 4562685-4562790, 4562949-4563815, 4563853-4564045, 4564146-4564574, 4564666-4588701, |
| 63 | MGRN1 | NM_001142290 | chr16 | 4675050-4700364, 4700485-4701962, 4702053-4702677, 4702826-4707245, 4707365-4714708, 4714777- |
| | | | | 4715101, 4715153-4718264, 4718314-4721390, 4721461-4723497, 4723659-4727462, 4727574- |
| | | | | 4730031, 4730099-4731549, 4731778-4732822, 4732948-4733223, 4733271-4733842, 4733934-4736258, |
| 63 | MGRN1 | NM_015246 | chr16 | 4675050-4700364, 4700485-4701962, 4702053-4702677, 4702826-4707245, 4707365-4714708, 4714777- |
| | | | | 4715101, 4715153-4718264, 4718314-4721390, 4721461-4723497, 4723659-4727462, 4727574- |
| | | | | 4730031, 4730099-4731549, 4731778-4732822, 4732948-4733223, 4733271-4733842, 4733934-4738795, |
| 63 | MGRN1 | NM_001142289 | chr16 | 4675050-4700364, 4700485-4701962, 4702053-4702677, 4702826-4707245, 4707365-4714708, 4714777- |
| | | | | 4715098, 4715153-4718264, 4718314-4721390, 4721461-4723497, 4723659-4727462, 4727574- |
| | | | | 4731549, 4731778-4732822, 4732948-4733223, 4733271-4733842, 4733934-4738795, |
| 63 | MGRN1 | NM_001142291 | chr16 | 4675050-4700364, 4700485-4701962, 4702053-4702677, 4702826-4707245, 4707365-4714708, 4714777- |
| | | | | 4715098, 4715153-4718264, 4718314-4721390, 4721461-4723497, 4723659-4727462, 4727574- |
| | | | | 4731549, 4731778-4732822, 4732948-4733223, 4733271-4733842, 4733934-4736258, |
| 63 | C16orf71 | NM_139170 | chr16 | 4784700-4786463, 4786645-4787799, 4787948-4790152, 4790661-4793042, 4793162-4794869, 4795013- |
| | | | | 4796374, 4796514-4796926, 4797027-4797342, 4797636-4798724, |
| 63 | LOC440335 | NR_029453 | chr16 | 4838773-4844951, 4845078-4845714, 4845860-4845947, 4846035-4846189, |
| 63 | LOC440335 | NR_029454 | chr16 | 4838581-4838728, 4838773-4844951, 4845078-4845714, 4845860-4845947, 4846050-4846189, |
| 63 | LOC440335 | NR_029455 | chr16 | 4845451-4845714, 4845860-4845947, 4846035-4846189, |
| 63 | NAGPA | NM_016256 | chr16 | 5075687-5077134, 5077200-5077277, 5077381-5077845, 5077895-5077979, 5078187-5078879, 5079010- |
| | | | | 5080384, 5080495-5081744, 5081886-5083272, 5083730-5083828, |
| 63 | ABAT | NM_000663 | chr16 | 8807723-8829554, 8829667-8839856, 8839956-8841963, 8841995-8844277, 8844397-8851612, 8851664- |
| | | | | 8857924, 8858007-8858593, 8858688-8860063, 8860128-8862048, 8862114-8862680, 8862831- |
| | | | | 8866635, 8866775-8868745, 8868915-8870199, 8870348-8873334, 8873448-8875164, |
| 63 | ABAT | NM_020686 | chr16 | 8768569-8829554, 8829667-8839856, 8839956-8841963, 8841995-8844277, 8844397-8851612, 8851664- |
| | | | | 8857924, 8858007-8858593, 8858688-8860063, 8860128-8862048, 8862114-8862680, 8862831- |
| | | | | 8866635, 8866775-8868745, 8868915-8870199, 8870348-8873334, 8873448-8875164, |
| 63 | ABAT | NM_001127448 | chr16 | 8814792-8829554, 8829667-8839856, 8839956-8841963, 8841995-8844277, 8844397-8851612, 8851664- |
| | | | | 8857924, 8858007-8858593, 8858688-8860063, 8860128-8862048, 8862114-8862680, 8862831- |
| | | | | 8866635, 8866775-8868745, 8868915-8870199, 8870348-8873334, 8873448-8875164, |
| 63 | NUBP1 | NM_002484 | chr16 | 10837740-10837816, 10837923-10840998, 10841134-10846465, 10846536-10849576, 10849611- |
| | | | | 10850545, 10850638-10851728, 10851885-10855219, 10855332-10855612, 10855717-10861804, 10861890- |
| | | | | 10862902, |
| 63 | CLEC16A | NM_015226 | chr16 | 11038655-11051637, 11051768-11056310, 11056446-11063016, 11063167-11064980, 11065088- |
| | | | | 11066787, 11066919-11071080, 11071257-11073184, 11073240-11076733, 11076849-11096929, 11097163- |
| | | | | 11114048, 11114183-11118676, 11118779-11133609, 11133734-11136109, 11136221-11137883, 11137930- |
| | | | | 11141147, 11141200-11145368, 11145499-11154757, 11154880-11214470, 11214624-11217597, 11217804- |
| | | | | 11219834, 11220004-11260243, 11260410-11272190, |
| 63 | MKL2 | NM_014048 | chr16 | 14165223-14173145, 14173212-14234399, 14234618-14304098, 14304166-14306209, 14306267- |
| | | | | 14307400, 14307478-14310981, 14311145-14312675, 14312856-14328001, 14328141-14334092, 14334342- |
| | | | | 14339383, 14339518-14340328, 14341215-14342781, 14342939-14345717, 14345881-14346220, 14346360- |
| | | | | 14351956, 14352019-14354764, |
| 63 | NOMO2 | NM_001004060 | chr16 | 18511367-18511696, 18511826-18512247, 18512342-18518129, 18518251-18520485, 18520589- |
| | | | | 18522865, 18522978-18524670, 18524756-18525662, 18525806-18527203, 18527333-18528509, 18528625- |
| | | | | 18530549, 18530678-18530821, 18530983-18531917, 18532002-18532085, 18532306-18535164, 18535262- |
| | | | | 18535809, 18535875-18538857, 18538947-18540821, 18540960-18542357, 18542491-18542786, 18542930- |
| | | | | 18544325, 18544502-18549846, 18549999-18550179, 18550287-18553492, 18553584-18553914, 18554054- |
| | | | | 18554937, 18555092-18558548, 18558623-18560841, 18560950-18562759, 18562862-18566127, 18566175- |
| | | | | 18569022, 18569114-18573196, |
| 63 | NOMO2 | NM_173614 | chr16 | 18511826-18512247, 18512342-18518129, 18518251-18520485, 18520589-18522865, 18522978- |
| | | | | 18524670, 18524756-18525662, 18525806-18527203, 18527333-18528509, 18528625-18530549, 18530678- |
| | | | | 18530821, 18530983-18531917, 18532002-18532085, 18532306-18535164, 18535262-18535809, 18535875- |
| | | | | 18538857, 18538947-18540821, 18540960-18542357, 18542491-18542786, 18542930-18544325, 18544502- |
| | | | | 18549846, 18549999-18550179, 18550287-18553492, 18553584-18553914, 18554054-18554937, 18555092- |
| | | | | 18558548, 18558623-18560841, 18560950-18562759, 18562862-18566127, 18566175-18569022, 18569114- |
| | | | | 18573196, |
| 63 | ACSM5 | NM_017888 | chr16 | 20420988-20422790, 20423011-20429379, 20429592-20430548, 20430758-20432578, 20432724- |
| | | | | 20435236, 20435392-20439108, 20439190-20440998, 20441124-20442313, 20442396-20442540, 20442644- |
| | | | | 20448372, 20448502-20448588, 20448690-20451120, 20451242-20451664, |
| 63 | LYRM1 | NM_020424 | chr16 | 20911858-20926876, 20927037-20931442, 20931537-20935320, |
| 63 | LYRM1 | NM_001128301 | chr16 | 20912212-20913806, 20914040-20926876, 20927037-20931442, 20931537-20935320, |
| 63 | LYRM1 | NM_001128302 | chr16 | 20912212-20926876, 20927037-20931442, 20931537-20935320, |
| 63 | C16orf52 | NM_001164579 | chr16 | 22019729-22086806, 22087021-22092015, |
| 63 | EEF2K | NM_013302 | chr16 | 22218001-22236973, 22237297-22255949, 22256052-22260074, 22260137-22261973, 22262013- |
| | | | | 22262470, 22262644-22268067, 22268219-22268572, 22268707-22268962, 22269092-22269813, 22270017- |
| | | | | 22271781, 22271851-22274429, 22274509-22276137, 22276202-22277709, 22277846-22278007, 22278198- |
| | | | | 22284945, 22285072-22291517, 22291698-22295206, |
| 63 | RBBP6 | NM_006910 | chr16 | 24552114-24557482, 24557584-24560264, 24560303-24564832, 24564879-24566934, 24567025- |
| | | | | 24567140, 24567239-24567627, 24567769-24570824, 24570999-24572931, 24573037-24573143, 24573483- |
| | | | | 24574518, 24574617-24574742, 24574823-24574929, 24574986-24576116, 24576187-24578462, 24578827- |
| | | | | 24579111, 24579215-24580064, 24581821-24582195, |
| 63 | RBBP6 | NM_032626 | chr16 | 24552114-24557482, 24557584-24560264, |
| 63 | RBBP6 | NM_018703 | chr16 | 24552114-24557482, 24557584-24560264, 24560303-24564832, 24564879-24566934, 24567025- |
| | | | | 24567140, 24567239-24567627, 24567769-24570824, 24570999-24572931, 24573037-24573143, 24573483- |
| | | | | 24574518, 24574617-24574742, 24574823-24574929, 24574986-24576116, 24576187-24578462, 24578827- |
| | | | | 24580064, 24581821-24582195, |
| 63 | NSMCE1 | NM_145080 | chr16 | 27236558-27237045, 27237168-27238039, 27238158-27244320, 27244469-27245507, 27245587- |
| | | | | 27246497, 27246621-27268754, 27268903-27280025, |
| 63 | FLJ21408 | NR_037184 | chr16 | 27279566-27292855, 27292957-27297981, 27298066-27300119, 27300275-27301564, |
| 63 | CCDC101 | NM_138414 | chr16 | 28565419-28592374, 28592466-28596232, 28596310-28596967, 28597042-28600419, 28600486- |
| | | | | 28601415, 28601547-28601615, 28601764-28601950, 28601988-28602092, 28602257-28602909, |
| 63 | SH2B1 | NM_015503 | chr16 | 28878355-28878650, 28878754-28880325, 28880419-28880527, 28880705-28883099, 28883305- |
| | | | | 28883509, 28883723-28883853, 28884027-28884489, 28884591-28884766, |
| 63 | SH2B1 | NM_001145812 | chr15 | 28875584-28877331, 28878355-28878650, 28878754-28880325, 28880419-28880527, 28880705- |
| | | | | 28883099, 28883305-28883509, 28883723-28883853, 28884027-28884489, 28884591-28884766, |
| 63 | SH2B1 | NM_001145796 | chr16 | 28875267-28877331, 28878355-28878650, 28878754-28880325, 28880419-28880527, 28880705- |
| | | | | 28883099, 28883305-28883509, 28883723-28883853, 28884027-28884489, 28884591-28884766, |
| 63 | SH2B1 | NM_001145795 | chr16 | 28875267-28877331, 28878355-28878650, 28878754-28880325, 28880419-28880527, 28880705- |
| | | | | 28883099, 28883305-28883509, 28883723-28883853, 28884027-28884766, |
| 63 | SH2B1 | NM_001145797 | chr16 | 28875267-28877331, 28878355-28878650, 28878754-28880325, 28880419-28880527, 28880705- |
| | | | | 28883099, 28883305-28883509, 28883723-28883853, 28884080-28884489, 28884591-28884766, |
| 64 | MVP | NM_005115 | chr16 | 29831832-29841834, 29841996-29842197, 29842395-29845052, 29845178-29845254, 29845388- |
| | | | | 29847023, 29847120-29848041, 29848280-29851497, 29851781-29852915, 29853162-29853234, 29853434- |
| | | | | 29855812, 29856201-29857182, 29857301-29857477, 29857606-29858516, 29858707-29859081, |
| 64 | MVP | NM_017458 | chr16 | 29831832-29841793, 29841996-29842197, 29842395-29845052, 29845178-29845254, 29845388- |
| | | | | 29847023, 29847120-29848041, 29848280-29851497, 29851781-29852915, 29853162-29853234, 29853434- |
| | | | | 29855812, 29856201-29857182, 29857301-29857477, 29857606-29858516, 29858707-29859081, |
| 64 | LOC595101 | NR_002453 | chr16 | 30278966-30279786, 30279812-30280316, 30280405-30281375, 30281487-30284348, 30284552- |
| | | | | 30284698, 30284843-30287332, 30287489-30288583, 30288708-30288916, 30289115-30289500, 30289702- |
| | | | | 30290252, 30290395-30291012, 30291293-30291762, 30291788-30292628, 30292747-30292894, 30293027- |
| | | | | 30296388, 30296779-30297486, 30297590-30299880, 30300107-30302522, 30302697-30304418, 30304518- |
| | | | | 30305434, 30305509-30305885, 30306013-30309696, 30309912-30311188, 30311249-30312541, 30312680- |
| | | | | 30316374, 30316532-30317109, 30317275-30346294, |
| 64 | CD2BP2 | NR_024465 | chr16 | 30364417-30364500, 30364609-30364687, 30365122-30365220, 30365380-30365503, 30365644-30365923, |
| 64 | CD2BP2 | NM_006110 | chr16 | 30364417-30364500, 30364609-30364687, 30365122-30365220, 30365380-30365503, 30365644- |
| | | | | 30365923, 30366029-30366531, |
| 64 | TBC1D10B | NM_015527 | chr16 | 30369895-30369982, 30370139-30370491, 30370683-30370857, 30370925-30371046, 30371163- |
| | | | | 30376219, 30376328-30376424, 30376534-30376814, 30376916-30380547, |
| 64 | SEPT1 | NM_052838 | chr16 | 30389842-30389929, 30390022-30390337, 30390505-30390754, 30390856-30391251, 30391355- |
| | | | | 30392446, 30392566-30392658, 30392795-30393079, 30393205-30393412, 30393501-30393594, 30393687- |
| | | | | 30393844, 30393920-30394054, |
| 64 | ZNF768 | NM_024671 | chr16 | 30537373-30537645, |
| 64 | PHKG2 | NM_001172432 | chr16 | 30759812-30760122, 30760237-30762425, 30762603-30762868, 30762925-30764551, 30764619- |
| | | | | 30764713, 30764879-30767501, 30767594-30767686, 30767842-30767909, 30768037-30768123, 30768281- |
| | | | | 30771438, |
| 64 | PHKG2 | NM_000294 | chr16 | 30759812-30760122, 30760237-30762425, 30762603-30762868, 30762925-30764551, 30764619- |
| | | | | 30764713, 30764879-30767501, 30767594-30767686, 30767842-30767909, 30768037-30768123, |
| 64 | BCL7C | NM_004765 | chr16 | 30899312-30900179, 30900267-30903905, 30904069-30904159, 30904270-30904531, 30904612-30905172, |
| 64 | STX1B | NM_052874 | chr16 | 31004223-31004449, 31004562-31004666, 31004806-31008002, 31008078-31008270, 31008381- |
| | | | | 31008821, 31008897-31012247, 31012324-31012413, 31012515-31012848, 31012925-31021686, |
| 64 | C16orf58 | NM_022744 | chr16 | 31502254-31503328, 31503408-31503516, 31503662-31504297, 31504370-31504786, 31504846- |
| | | | | 31504922, 31505109-31505185, 31505258-31508168, 31508272-31510621, 31510729-31510812, 31510847- |
| | | | | 31512005, 31512053-31519083, 31519200-31519393, |
| 65 | ZNF721 | NM_133474 | chr4 | 438222-466362, 466491-492843, |
| 65 | PIGG | NM_001127178 | chr4 | 493279-494183, 494391-499505, 499717-501192, 501383-502616, 502760-509760, 509975-514843, 515063- |
| | | | | 515447, 515731-517246, 517703-520826, 521020-524223, 524535-527605, 527771-532940, |
| 65 | PIGG | NM_017733 | chr4 | 493279-494183, 494391-499505, 499717-501192, 501383-502616, 502760-509760, 509975-514843, 515039- |
| | | | | 515447, 515731-517246, 517703-520826, 521020-524223, 524535-527605, 527771-532940, |
| 65 | PDE6B | NM_001145291 | chr4 | 619884-628464, 628619-629667, 629759-647639, 647782-647867, 647944-648611, 648678-649727, 649796- |
| | | | | 650032, 650082-650661, 650813-651138, 651284-652739, 652807-654254, 654403-655921, 656031- |
| | | | | 656296, 656408-656887, 656977-657557, 657660-657901, 658011-658668, 658734-659042, 659119- |
| | | | | 660318, 660404-661643, 661796-663836, |
| 65 | PDE6B | NM_001145292 | chr4 | 647109-647639, 647782-647867, 647944-648611, 648678-649727, 649796-650032, 650082-650661, 650813- |
| | | | | 651138, 651284-652739, 652807-654254, 654403-655921, 656031-656296, 656408-656887, 656977- |
| | | | | 657557, 657660-657901, 658011-658668, 658734-659042, 659119-660318, 660404-661643, 661796-663833, |
| 65 | PDE6B | NM_000283 | chr4 | 619884-628464, 628619-629667, 629759-647639, 647782-647867, 647944-648611, 648678-649727, 649796- |
| | | | | 650032, 650082-650661, 650813-651138, 651284-652739, 652807-654254, 654403-655921, 656031- |
| | | | | 656296, 656408-656887, 656977-657557, 657660-657901, 658011-658668, 658734-659042, 659119- |
| | | | | 660318, 660404-661643, 661796-663833, |
| 65 | PCGF3 | NM_006315 | chr4 | 699760-724417, 724458-724757, 724900-727459, 727579-728718, 728817-731253, 731311-737260, 737373- |
| | | | | 738386, 738477-755065, 755205-758770, 758853-759818, |
| 65 | FGFRL1 | NM_021923 | chr4 | 1006353-1015989, 1016264-1017427, 1017510-1017603, 1017890-1018097, 1018453-1018691, |
| 65 | FGFRL1 | NM_001004358 | chr4 | 1005776-1006256, 1006353-1015989, 1016264-1017427, 1017510-1017603, 1017890-1018097, 1018453- |
| | | | | 1018691, |
| 65 | FGFRL1 | NM_001004356 | chr4 | 1005743-1006256, 1006353-1015989, 1016264-1017427, 1017510-1017603, 1017890-1018097, 1018453- |
| | | | | 1018691, |
| 65 | TNIP2 | NM_024309 | chr4 | 2744248-2746112, 2746234-2746422, 2746673-2747171, 2747263-2749380, 2749673-2757739, |
| 65 | TNIP2 | NM_001161527 | chr4 | 2744248-2746112, 2746234-2746422, 2746673-2747171, 2747263-2749380, 2749673-2757674, |
| 65 | C4orf44 | NM_001042690 | chr4 | 3251270-3254932, 3255210-3257450, |
| 65 | RGS12 | NM_002926 | chr4 | 3316677-3317795, 3319779-3344662, 3344781-3388141, 3388165-3415797, 3415969-3416477, 3416572- |
| | | | | 3417703, 3417849-3418638, 3418820-3419113, 3419269-3422367, 3422446-3424101, 3424298- |
| | | | | 3424630, 3424706-3425240, 3425369-3427189, 3427288-3429815, 3429897-3430283, 3430439-3432132, |
| 65 | RGS12 | NM_198229 | chr4 | 3316677-3317795, 3319779-3344662, 3344781-3388141, 3388165-3415797, 3415969-3416477, 3416572- |
| | | | | 3417703, 3417849-3418638, 3418820-3419113, 3419269-3422367, 3422446-3424101, 3424298- |
| | | | | 3424630, 3424706-3425240, 3425369-3427189, 3427288-3429815, 3429897-3430283, 3430439- |
| | | | | 3432132, 3432683-3441180, |
| 65 | RGS12 | NM_198227 | chr4 | 3372049-3388141, 3388165-3415797, 3415969-3416477, 3416572-3417703, 3417849-3418638, 3418820- |
| | | | | 3419113, 3419269-3422367, 3422446-3424101, 3424298-3424630, 3424706-3425240, 3425369- |
| | | | | 3427189, 3427288-3429815, 3429897-3430283, 3430439-3432132, 3432683-3441180, |
| 65 | LRPAP1 | NM_002337 | chr4 | 3514764-3516477, 3516656-3517841, 3517926-3519759, 3519920-3520596, 3520719-3521797, 3521921- |
| | | | | 3526632, 3526779-3533934, |
| 65 | STK32B | NM_018401 | chr4 | 5053643-5141630, 5141688-5170024, 5170178-5332945, 5333121-5399932, 5399972-5418570, 5418662- |
| | | | | 5448398, 5448504-5458532, 5458651-5461828, 5461956-5468428, 5468562-5469731, 5469798-5500670, |
| 65 | CRMP1 | NM_001014809 | chr4 | 5823579-5827219, 5827387-5830214, 5830396-5837640, 5837813-5838490, 5838634-5841247, 5841406- |
| | | | | 5843033, 5843156-5844818, 5844889-5851117, 5851200-5853133, 5853197-5857868, 5858035- |
| | | | | 5862751, 5862938-5868393, 5868484-5894314, |
| 65 | CRMP1 | NM_001313 | chr4 | 5823579-5827219, 5827387-5830214, 5830396-5837640, 5837813-5838490, 5838634-5841247, 5841406- |
| | | | | 5843033, 5843156-5844818, 5844889-5851117, 5851200-5853133, 5853197-5857868, 5858035- |
| | | | | 5862751, 5862938-5868393, 5868484-5889953, |
| 65 | WFS1 | NM_001145853 | chr4 | 6271742-6279180, 6279415-6288818, 6288903-6290712, 6290859-6292922, 6293095-6293642, 6293725- |
| | | | | 6296766, 6296917-6302382, |
| 65 | WFS1 | NM_006005 | chr4 | 6271742-6279176, 6279415-6288818, 6288903-6290712, 6290859-6292922, 6293095-6293642, 6293725- |
| | | | | 6296766, 6296917-6302382, |
| 65 | MAN2B2 | NM_015274 | chr4 | 6577076-6578303, 6578452-6580118, 6580226-6588721, 6588896-6590770, 6590888-6594898, 6595078- |
| | | | | 6596259, 6596460-6598838, 6599031-6599923, 6600082-6602348, 6602484-6606780, 6607057- |
| | | | | 6610832, 6611026-6611523, 6611778-6612605, 6612718-6612811, 6613006-6615943, 6616083- |
| | | | | 6619105, 6619220-6621652, 6621772-6622913, |
| 65 | LOC100129931 | NR_033828 | chr4 | 7035781-7036896, 7037002-7046705, 7046830-7047384, |
| 65 | GRPEL1 | NM_025196 | chr4 | 7062936-7064110, 7064194-7065786, 7065951-7069696, |
| 66 | BAI2 | NM_001703 | chr1 | 32193207-32193624, 32193683-32193781, 32193882-32194197, 32194235-32196399, 32197021- |
| | | | | 32197714, 32197756-32198116, 32198214-32198571, 32198745-32200782, 32200883-32201179, 32201267- |
| | | | | 32201426, 32201495-32201920, 32201992-32202172, 32202325-32203028, 32203133-32203251, 32203357- |
| | | | | 32203799, 32203928-32204186, 32204232-32204431, 32204539-32204907, 32205237-32205525, 32205636- |
| | | | | 32205709, 32205780-32205946, 32206143-32206972, 32207081-32207223, 32207327-32207399, 32207569- |
| | | | | 32207652, 32207819-32208437, 32208604-32209792, 32209959-32210247, 32210333-32221598, 32222417- |
| | | | | 32222800, 32222883-32222981, 32223113-32229483, |
| 66 | CCDC28B | NM_024296 | chr1 | 32666311-32667512, 32667701-32669478, 32669647-32669785, 32669981-32670197, 32670222-32670793, |
| 66 | IQCC | NM_018134 | chr1 | 32671325-32671753, 32671899-32672108, 32672363-32672601, 32672722-32672839, |
| 66 | IQCC | NM_001160042 | chr1 | 32671565-32671753, 32671899-32672108, 32672363-32672601, 32672722-32672839, |
| 66 | MTMR9LP | NR_026850 | chr1 | 32697571-32697641, 32697786-32700303, 32700416-32700551, 32700812-32704894, 32705027- |
| | | | | 32705468, 32706266-32706736, |
| 66 | ADC | NM_052998 | chr1 | 33546896-33546987, 33547110-33547200, 33547414-33547777, 33547956-33549553, 33549729- |
| | | | | 33557649, 33557824-33558881, 33559018-33560147, 33560315-33562306, 33562471-33563666, 33563781- |
| | | | | 33583501, 33583718-33585643, |
| 66 | TEKT2 | NM_014466 | chr1 | 36549751-36550469, 36550679-36550762, 36550890-36551435, 36551643-36552303, 36552449- |
| | | | | 36552530, 36552647-36552803, 36552913-36553038, 36553184-36553327, 36553409-36553572, |
| 66 | MAP7D1 | NM_018067 | chr1 | 36622065-36636570, 36636917-36637112, 36637183-36638063, 36638229-36638963, 36639080- |
| | | | | 36640497, 36640610-36641798, 36642183-36642296, 36642444-36643472, 36643803-36644018, 36644198- |
| | | | | 36644284, 36644425-36644520, 36644627-36644861, 36644917-36645111, 36645159-36645265, 36645358- |
| | | | | 36645473, 36645669-36645847, |
| 66 | OSCP1 | NM_145047 | chr1 | 36883857-36884590, 36884656-36886075, 36886217-36887749, 36887821-36888367, 36888498- |
| | | | | 36888962, 36889068-36897401, 36897484-36898021, 36898191-36904355, 36904512-36915857, |
| 66 | OSCP1 | NM_206837 | chr1 | 36894077-36897401, 36897484-36898021, 36898191-36904355, 36904512-36915857, |
| 66 | DNALI1 | NM_003462 | chr1 | 38022677-38023202, 38023350-38024926, 38025098-38027156, 38027337-38027680, 38027847-38030625, |
| 66 | RSPO1 | NM_001038633 | chr1 | 38077350-38077419, 38078594-38079374, 38079565-38079854, 38080006-38082154, 38082348- |
| | | | | 38095238, 38095622-38097514, 38097695-38097957, 38098026-38100236, |
| 67 | STON2 | NM_033104 | chr14 | 81737217-81743243, 81745084-81837330, 81837530-81862236, 81862523-81864637, |
| 67 | GALC | NM_000153 | chr14 | 88401223-88406247, 88406326-88407737, 88407903-88411895, 88412078-88414070, 88414223- |
| | | | | 88416187, 88416276-88417001, 88417093-88429726, 88429856-88431847, 88431974-88434677, 88434835- |
| | | | | 88442700, 88442833-88448547, 88448588-88450736, 88450878-88452831, 88452947-88454486, 88454552- |
| | | | | 88454797, 88454868-88459312, |
| 67 | GALC | NM_001037525 | chr14 | 88429856-88431847, 88431974-88434677, 88434835-88442700, 88442833-88448547, 88448588- |
| | | | | 88450736, 88450878-88452831, 88452947-88454486, 88454552-88454797, 88454868-88459312, |
| 67 | TTC8 | NM_198309 | chr14 | 89291166-89305794, 89305917-89307207, 89307273-89307379, 89307541-89307767, 89307859- |
| | | | | 89310148, 89310195-89319313, 89319401-89323532, 89323622-89327564, 89327677-89336401, 89336543- |
| | | | | 89337891, 89338068-89338672, 89338797-89341368, 89341454-89343636, |
| 67 | TTC8 | NM_198310 | chr14 | 89291166-89305794, 89305917-89307207, 89307273-89307379, 89307541-89310148, 89310195- |
| | | | | 89319313, 89319401-89323532, 89323622-89327564, 89327677-89336401, 89336543-89337891, 89338068- |
| | | | | 89338672, 89338797-89341368, 89341454-89343636, |
| 67 | TTC8 | NM_144596 | chr14 | 89291166-89300035, 89300067-89305794, 89305917-89307207, 89307273-89307379, 89307541- |
| | | | | 89307767, 89307859-89310148, 89310195-89319313, 89319401-89323532, 89323622-89327564, 89327677- |
| | | | | 89336401, 89336543-89337891, 89338068-89338672, 89338797-89341368, 89341454-89343636, |
| 67 | KCNK13 | NM_022054 | chr14 | 90528884-90650453, |
| 67 | GPR68 | NM_001177676 | chr14 | 91701524-91710639, |
| 67 | GPR68 | NM_003485 | chr14 | 91701524-91720057, |
| 67 | ATXN3 | NR_028457 | chr14 | 92530759-92537277, 92537398-92545757, 92545813-92547307, 92547406-92548642, 92548811- |
| | | | | 92549468, 92549603-92549755, 92549848-92555072, 92555162-92559594, 92559663-92562435, 92562482- |
| | | | | 92563016, 92563183-92572871, |
| 67 | ATXN3 | NR_028455 | chr14 | 92530759-92537277, 92537398-92545757, 92545813-92547307, 92547406-92548642, 92548811- |
| | | | | 92549468, 92549603-92555072, 92555162-92559594, 92559663-92562435, 92562482-92563016, 92563183- |
| | | | | 92572871, |
| 67 | ATXN3 | NR_028462 | chr14 | 92530759-92537277, 92537398-92547307, 92547406-92548642, 92548811-92549468, 92549580- |
| | | | | 92555072, 92555162-92559594, 92559663-92562435, 92562482-92563016, 92563183-92572871, |
| 67 | ATXN3 | NR_028461 | chr14 | 92530759-92537277, 92537398-92547307, 92547406-92548642, 92548811-92549468, 92549603- |
| | | | | 92559594, 92559663-92560088, 92560176-92562435, 92562482-92563016, 92563183-92572871, |
| 67 | ATXN3 | NM_030660 | chr14 | 92530759-92537277, 92537398-92547307, 92547406-92548642, 92548811-92549468, 92549603- |
| | | | | 92555072, 92555162-92559594, 92559663-92560088, 92560176-92562435, 92562482-92572871, |
| 67 | ATXN3 | NM_001164774 | chr14 | 92530759-92537277, 92537398-92563016, 92563183-92572871, |
| 67 | ATXN3 | NM_001164778 | chr14 | 92530759-92537277, 92537398-92559594, 92559663-92560088, 92560176-92562435, 92562482- |
| | | | | 92563016, 92563183-92572871, |
| 67 | ATXN3 | NM_001164781 | chr14 | 92530759-92537277, 92537398-92547307, 92547406-92548642, 92548811-92549468, 92549603- |
| | | | | 92555072, 92555162-92559594, 92559663-92560088, 92560176-92572871, |
| 67 | ATXN3 | NM_001164782 | chr14 | 92530759-92537277, 92537398-92572871, |
| 67 | ATXN3 | NM_001164777 | chr14 | 92530759-92537277, 92537398-92562435, 92562482-92572871, |
| 67 | ATXN3 | NM_001164780 | chr14 | 92530759-92537277, 92537398-92547307, 92547406-92548642, 92548811-92549468, 92549603- |
| | | | | 92555072, 92555162-92559594, 92559663-92572871, |
| 67 | ATXN3 | NM_001164779 | chr14 | 92530759-92537277, 92537398-92547307, 92547406-92548642, 92548811-92549468, 92549603- |
| | | | | 92555072, 92555162-92572871, |
| 67 | ATXN3 | NR_028470 | chr14 | 92530759-92537277, 92537398-92559594, 92559663-92572871, |
| 67 | ATXN3 | NR_028469 | chr14 | 92530759-92537277, 92537398-92547295, 92547406-92548642, 92548811-92549468, 92549603- |
| | | | | 92555072, 92555162-92559594, 92559663-92562435, 92562482-92572871, |
| 67 | ATXN3 | NM_001127697 | chr14 | 92530759-92537277, 92537398-92547307, 92547406-92548642, 92548811-92549468, 92549603- |
| | | | | 92555072, 92555162-92562435, 92562482-92563016, 92563183-92572871, |
| 67 | ATXN3 | NR_028468 | chr14 | 92530759-92537277, 92537398-92547307, 92547406-92549468, 92549603-92555072, 92555162- |
| | | | | 92559594, 92559663-92562435, 92562482-92563016, 92563183-92572871, |
| 67 | ATXN3 | NR_028467 | chr14 | 92530759-92537277, 92537398-92547307, 92547406-92548642, 92548811-92549468, 92549580- |
| | | | | 92549755, 92549848-92555072, 92555162-92562435, 92562482-92563016, 92563183-92572871, |
| 67 | ATXN3 | NR_028466 | chr14 | 92530759-92537277, 92537398-92547307, 92547406-92548642, 92548811-92549468, 92549603-92572871, |
| 67 | ATXN3 | NR_028465 | chr14 | 92530759-92537277, 92537398-92545757, 92545813-92547307, 92547406-92548642, 92548811- |
| | | | | 92549468, 92549603-92555072, 92555162-92559594, 92559663-92563016, 92563183-92572871, |
| 67 | ATXN3 | NM_004993 | chr14 | 92530759-92537277, 92537398-92547307, 92547406-92548642, 92548811-92549468, 92549603- |
| | | | | 92555072, 92555162-92559594, 92559663-92560088, 92560176-92562435, 92562482-92563016, 92563183- |
| | | | | 92572871, |
| 67 | ATXN3 | NR_028460 | chr14 | 92530759-92537277, 92537398-92559594, 92559663-92562435, 92562482-92563016, 92563183-92572871, |
| 67 | ATXN3 | NR_028459 | chr14 | 92530759-92537277, 92537398-92545757, 92545813-92547307, 92547406-92548642, 92548811- |
| | | | | 92549468, 92549603-92555072, 92555162-92559594, 92559663-92560088, 92560176-92562435, 92562482- |
| | | | | 92563016, 92563183-92572871, |
| 67 | ATXN3 | NR_028458 | chr14 | 92530759-92537277, 92537398-92547295, 92547406-92548642, 92548811-92549468, 92549580- |
| | | | | 92555072, 92555162-92559594, 92559663-92562435, 92562482-92563016, 92563183-92572871, |
| 67 | ATXN3 | NM_001164776 | chr14 | 92530759-92537277, 92537398-92562435, 92562482-92563016, 92563183-92572871, |
| 67 | ATXN3 | NR_028453 | chr14 | 92530759-92537277, 92537398-92547307, 92547406-92548642, 92548811-92549468, 92549603- |
| | | | | 92555072, 92555162-92559594, 92559663-92562435, 92562482-92563016, 92563183-92572871, |
| 67 | ATXN3 | NR_028456 | chr14 | 92530759-92537277, 92537398-92545757, 92545813-92547307, 92547406-92548642, 92548811- |
| | | | | 92549468, 92549603-92555072, 92555162-92559594, 92559663-92562435, 92562482-92572871, |
| 67 | ATXN3 | NM_001127696 | chr14 | 92530759-92537277, 92537398-92547307, 92547406-92548642, 92548811-92549468, 92549603- |
| | | | | 92555072, 92555162-92559594, 92559663-92560088, 92560176-92563016, 92563183-92572871, |
| 67 | ATXN3 | NR_028454 | chr14 | 92530759-92537277, 92537398-92547307, 92547406-92548642, 92548811-92549468, 92549603- |
| | | | | 92555072, 92555162-92559594, 92559663-92562435, 92562482-92572871, |
| 67 | ATXN3 | NR_028464 | chr14 | 92530759-92537277, 92537398-92545757, 92545813-92547307, 92547406-92548642, 92548811- |
| | | | | 92549468, 92549603-92555072, 92555162-92562435, 92562482-92563016, 92563183-92572871, |
| 67 | ATXN3 | NR_031765 | chr14 | 92530759-92537277, 92537398-92547307, 92547406-92548642, 92548811-92572871, |
| 67 | ATXN3 | NR_028463 | chr14 | 92530759-92537277, 92537398-92545757, 92545813-92547307, 92547406-92548642, 92548811- |
| | | | | 92549468, 92549603-92572871, |
| 68 | FCGBP | NM_003890 | chr19 | 40354137-40354259, 40354533-40357375, 40357761-40360854, 40361056-40362716, 40363292- |
| | | | | 40363862, 40364436-40366026, 40366572-40367296, 40367915-40368301, 40368877-40370193, 40370533- |
| | | | | 40373943, 40374087-40376311, 40376513-40376629, 40377226-40380117, 40380712-40382281, 40382827- |
| | | | | 40383549, 40384168-40384560, 40385136-40386444, 40386784-40389650, 40389794-40391996, 40392198- |
| | | | | 40392314, 40392911-40395802, 40396397-40397965, 40398511-40399237, 40399856-40400248, 40400824- |
| | | | | 40402132, 40402472-40405917, 40406061-40407934, 40408136-40408252, 40408849-40411636, 40412231- |
| | | | | 40419595, 40420165-40421090, 40421697-40423977, 40424550-40430288, 40430640-40432964, 40434214- |
| | | | | 40440469, |
| 68 | CCDC97 | NM_052848 | chr19 | 41816262-41822287, 41822745-41825477, 41825758-41826244, 41826376-41828498, |
| 68 | B9D2 | NM_030578 | chr19 | 41860919-41863800, 41863928-41869335, 41869429-41869861, |
| 68 | TMEM91 | NM_001098823 | chr19 | 41882760-41884184, 41884425-41888675, 41888827-41889777, |
| 68 | TMEM91 | NM_001042595 | chr19 | 41883229-41884184, 41884425-41888675, 41888827-41889464, |
| 68 | TMEM91 | NM_001098821 | chr19 | 41882760-41884184, 41884425-41888675, 41888827-41889618, |
| 68 | TMEM91 | NM_001098822 | chr19 | 41882760-41884184, 41884425-41888675, 41888827-41889766, |
| 68 | TMEM91 | NM_001098825 | chr19 | 41870001-41884184, 41884425-41888675, 41888827-41889766, |
| 68 | TMEM91 | NM_001098824 | chr19 | 41882760-41884184, 41884425-41888675, 41888827-41889789, |
| 68 | BCKDHA | NM_001164783 | chr19 | 41903841-41916540, 41916722-41916826, 41916915-41919952, 41920063-41925038, 41925202- |
| | | | | 41928067, 41928276-41928535, 41928676-41928901, 41929075-41930341, |
| 68 | BCKDHA | NM_000709 | chr19 | 41903841-41916540, 41916722-41916826, 41916915-41919952, 41920063-41925038, 41925202- |
| | | | | 41928067, 41928276-41928532, 41928676-41928901, 41929075-41930341, |
| 68 | B3GNT8 | NM_198540 | chr19 | 41932716-41933187, 41933476-41934499, |
| 69 | IFRD1 | NM_001550 | chr7 | 112090838-112095816, 112095923-112096055, 112096142-112096967, 112097094- |
| | | | | 112098914, 112099074-112101919, 112101972-112102054, 112102235-112102323, 112102434- |
| | | | | 112108034, 112108171-112112272, 112112403-112112819, 112112917-112115483, |
| 69 | IFRD1 | NM_001007245 | chr7 | 112063488-112090561, 112090838-112095816, 112095923-112096055, 112096142- |
| | | | | 112096967, 112097094-112098914, 112099074-112101919, 112101972-112102054, 112102235- |
| | | | | 112102323, 112102434-112108034, 112108171-112112272, 112112403-112112819, 112112917- |
| | | | | 112115483, |
| 69 | IFRD1 | NM_001197080 | chr7 | 112063488-112095816, 112095923-112096055, 112096142-112096967, 112097094- |
| | | | | 112098914, 112099074-112101919, 112101972-112102054, 112102235-112102323, 112102434- |
| | | | | 112108034, 112108171-112112272, 112112403-112112819, 112112917-112115483, |
| 69 | IFRD1 | NM_001197079 | chr7 | 112092567-112095816, 112095923-112096055, 112096142-112096967, 112097094- |
| | | | | 112098914, 112099074-112101919, 112101972-112102054, 112102235-112102323, 112102434- |
| | | | | 112108034, 112108171-112112272, 112112403-112112819, 112112917-112115483, |
| 69 | C7orf53 | NM_001134468 | chr7 | 112121038-112124865, 112124999-112126976, 112127107-112129863, |
| 69 | C7orf53 | NM_182597 | chr7 | 112121402-112124865, 112124999-112126976412127107-112129863, |
| 69 | FAM3C | NM_014888 | chr7 | 120990605-120991195, 120991324-121000095, 121000182-121002949, 121003002- |
| | | | | 121004182, 121004243-121011352, 121011478-121012162, 121012194-121018962, 121019069- |
| | | | | 121023009, 121023065-121036214, |
| 69 | FAM3C | NM_001040020 | chr7 | 120990605-120991195, 120991324-121000095, 121000182-121002949, 121003002- |
| | | | | 121004182, 121004243-121011352, 121011478-121012162, 121012194-121018962, 121019069- |
| | | | | 121023009, 121023065-121036254, |
| 69 | LOC154860 | NR_036484 | chr7 | 121944323-121944625, 121944798-121945340, 121946317-121946568, 121946715- |
| | | | | 121947355, 121947543-121948539, 121948612-121949636, |
| 69 | SPAM1 | NM_153189 | chr7 | 123565407-123589924, 123590038-123593417, 123594579-123595049, 123595141-123599536, |
| 69 | SPAM1 | NM_001174044 | chr7 | 123565586-123589924, 123590038-123593417, 123594579-123595049, 123595141-123599536, |
| 69 | SPAM1 | NM_003117 | chr7 | 123565407-123589924, 123590038-123593417, 123594579-123595049, 123595141- |
| | | | | 123599536, 123599979-123610250, 123610351-123611059, |
| 69 | SPAM1 | NM_001174046 | chr7 | 123566119-123589924, 123590038-123593417, 123594579-123595049, 123595141-123599536, |
| 69 | SPAM1 | NM_001174045 | chr7 | 123566119-123587756, 123587843-123589924, 123590038-123593417, 123594579- |
| | | | | 123595049, 123595141-123599536, |
| 69 | SND1 | NM_014390 | chr7 | 127292506-127326665, 127326817-127334880, 127335003-127338927, 127339008- |
| | | | | 127341215, 127341378-127342487, 127342581-127343217, 127343378-127344891, 127345000- |
| | | | | 127347609, 127347702-127361339, 127361455-127447536, 127447628-127484375, 127484478- |
| | | | | 127527953, 127528066-127544798, 127544873-127569239, 127569383-127630998, 127631110- |
| | | | | 127714552, 127714743-127721410, 127721554-127724774, 127724900-127725761, 127725833- |
| | | | | 127726988, 127727104-127729539, 127729745-127731887, 127731934-127732043, |
| 69 | CALU | NM_001130674 | chr7 | 128379487-128388625, 128388859-128394581, 128394777-128398923, 128399092- |
| | | | | 128399844, 128399907-128407508, 128407710-128409115, |
| 69 | CALU | NM_001219 | chr7 | 128379487-128388625, 128388859-128394314, 128394510-128398923, 128399092- |
| | | | | 128399844, 128399907-128407508, 128407710-128409115, |
| 69 | CALU | NM_001199671 | chr7 | 128379487-128387605, 128388422-128388625, 128388859-128394314, 128394510- |
| | | | | 128398923, 128399092-128399844, 128399907-128407508, 128407710-128409115, |
| 69 | CALU | NM_001199674 | chr7 | 128379487-128398923, 128399092-128399844, 128399907-128407508, 128407710-128409115, |
| 69 | CALU | NM_001199673 | chr7 | 128379487-128388625, 128388859-128394314, 128394510-128398923, 128399092- |
| | | | | 128399844, 128399907-128409115, |
| 69 | CALU | NM_001199672 | chr7 | 128379487-128387605, 128388422-128388625, 128388859-128394581, 128394777- |
| | | | | 128398923, 128399092-128399844, 128399907-128407508, 128407710-128409115, |
| 70 | ZNF189 | NM_197977 | chr9 | 104161472-104161675, 104161813-104162206, 104162293-104170209, |
| 70 | ZNF189 | NM_003452 | chr9 | 104161472-104162164, 104162293-104170209, |
| 70 | FSD1L | NM_001145313 | chr9 | 108210517-108223799, 108223897-108226515, 108226613-108230472, 108230606- |
| | | | | 108234183, 108234287-108241633, 108241658-108246660, 108246784-108268678, 108268890- |
| | | | | 108270896, 108270997-108275086, 108275218-108296772, 108296875-108297346, 108297600- |
| | | | | 108301542, 108301633-108308637, |
| 70 | FSD1L | NM_031919 | chr9 | 108210517-108226515, 108226613-108230472, 108230606-108234183, |
| 70 | FSD1L | NM_207647 | chr9 | 108210517-108226515, 108226613-108230472, 108230606-108234183, 108234287- |
| | | | | 108241633, 108241658-108246660, 108246784-108268678, 108268890-108270896, 108270997- |
| | | | | 108275086, 108275218-108296772, 108296875-108297346, 108297600-108301542, 108301633- |
| | | | | 108308637, |
| 70 | IKBKAP | NM_003640 | chr9 | 111631463-111637173, 111637251-111640273, 111640430-111640901, 111641031- |
| | | | | 111641724, 111641838-111642330, 111642446-111643983, 111644046-111644403, 111644468- |
| | | | | 111651610, 111651674-111653481, 111653685-111655264, 111655364-111656221, 111656347- |
| | | | | 111658774, 111658925-111659231, 111659319-111659426, 111659566-111660780, 111660862- |
| | | | | 111660949, 111661030-111662100, 111662176-111662538, 111662656-111663703, 111663811- |
| | | | | 111663906, 111663962-111665117, 111665223-111665841, 111665950-111668581, 111668766- |
| | | | | 111670583, 111670685-111673288, 111673461-111674542, 111674775-111678482, 111678578- |
| | | | | 111679825, 111679951-111681089, 111681182-111681531, 111681630-111685120, 111685208- |
| | | | | 111688801, 111688884-111689650, 111689734-111692047, 111692202-111693275, 111693482- |
| | | | | 111696142, |
| 70 | PTGR1 | NM_001146108 | chr9 | 114325512-114332369, 114332490-114337012, 114337123-114341074, 114341232- |
| | | | | 114345750, 114345870-114348276, 114348446-114355204, 114355263-114356500, 114356548- |
| | | | | 114359595, 114359713-114361881, |
| 70 | PTGR1 | NM_012212 | chr9 | 114325512-114332369, 114332490-114337012, 114337123-114341074, 114341232- |
| | | | | 114345750, 114345870-114348276, 114348446-114355204, 114355263-114356500, 114356548- |
| | | | | 114359595, 114359713-114361617, |
| 70 | PTGR1 | NM_001146109 | chr9 | 114312080-114332369, 114332490-114337012, 114337123-114341074, 114341232- |
| | | | | 114345750, 114345870-114348276, 114348446-114355204, 114355263-114356500, 114356548- |
| | | | | 114359595, 114359713-114361881, |
| 70 | C9orf80 | NM_021218 | chr9 | 115449914-115451805, 115451898-115456409, 115456514-115478739, 115478822-115480265, |
| 70 | C9orf43 | NM_152786 | chr9 | 116173419-116175723, 116175925-116176037, 116176175-116181386, 116181446- |
| | | | | 116183381, 116183484-116184827, 116184866-116185604, 116185779-116186444, 116186597- |
| | | | | 116187297, 116187357-116187622, 116187701-116187921, 116188011-116190425, 116190504- |
| | | | | 116191178, 116191244-116191450, |
| 71 | CRNN | NM_016190 | chr1 | 152383420-152384570, 152384723-152386688, |
| 71 | LCE3E | NM_178435 | chr1 | 152538706-152539212, |
| 71 | LCE1C | NM_178351 | chr1 | 152777975-152779075, |
| 71 | SPRR1B | NM_003125 | chr1 | 153003724-153004801, |
| 71 | S100A8 | NM_002964 | chr1 | 153362720-153362869, 153363034-153363515, |
| 71 | ILF2 | NM_004515 | chr1 | 153635033-153635179, 153635272-153635493, 153635610-153635689, 153635753- |
| | | | | 153636517, 153636607-153636870, 153636951-153637694, 153637813-153638137, 153638205- |
| | | | | 153640029, 153640134-153640474, 153640554-153640927, 153641034-153642310, 153642355- |
| | | | | 153642645, 153642707-153643392, |
| 72 | NAA15 | NM_057175 | chr4 | 140222986-140255326, 140255413-140258000, 140258107-140262064, 140262224- |
| | | | | 140263978, 140264115-140265338, 140265494-140270614, 140270736-140272343, 140272441- |
| | | | | 140272657, 140272766-140275178, 140275253-140278538, 140278710-140280895, 140281050- |
| | | | | 140281652, 140281783-140282876, 140283092-140291363, 140291559-140297517, 140297628- |
| | | | | 140299908, 140300009-140305984, 140306133-140307285, 140307385-140309036, |
| 72 | SLC10A7 | NM_001029998 | chr4 | 147177673-147179842, 147179990-147204342, 147204418-147214079, 147214133- |
| | | | | 147215080, 147215248-147227076, 147227162-147247111, 147247149-147363933, 147363974- |
| | | | | 147424999, 147425077-147431063, 147431202-147438188, 147438273-147442768, |
| 72 | SLC10A7 | NM_032128 | chr4 | 147363974-147424999, 147425077-147431063, 147431202-147438188, 147438273-147442768, |
| 72 | FBXW7 | NM_001013415 | chr4 | 153244302-153245334, 153245547-153247156, 153247384-153249358, 153249542- |
| | | | | 153250822, 153250938-153251882, 153252021-153253746, 153253872-153258952, 153259089- |
| | | | | 153268080, 153268224-153271192, 153271277-153303339, |
| 72 | FBXW7 | NM_018315 | chr4 | 153244302-153245334, 153245547-153247156, 153247384-153249358, 153249542- |
| | | | | 153250822, 153250938-153251882, 153252021-153253746, 153253872-153258952, 153259089- |
| | | | | 153268080, 153268224-153271192, 153271277-153273620, |
| 72 | FBXW7 | NM_033632 | chr4 | 153244302-153245334, 153245547-153247156, 153247384-153249358, 153249542- |
| | | | | 153250822, 153250938-153251882, 153252021-153253746, 153253872-153258952, 153259089- |
| | | | | 153268080, 153268224-153271192, 153271277-153332453, 153333025-153456091, |
| 72 | FGB | NM_001184741 | chr4 | 155484285-155486958, 155487011-155487675, 155487825-155488743, 155488973- |
| | | | | 155489531, 155489647-155490332, 155490460-155490664, 155490952-155491569, |
| 72 | FGB | NM_005141 | chr4 | 155484285-155486958, 155487152-155487639, 155487825-155488743, 155488973- |
| | | | | 155489531, 155489647-155490332, 155490460-155490664, 155490952-155491569, |
| 72 | FGG | NM_021870 | chr4 | 155526219-155527855, 155528135-155529616, 155529803-155530780, 155530916- |
| | | | | 155531217, 155531350-155532955, 155533051-155533168, 155533354-155533541, 155533588- |
| | | | | 155533682, |
| 72 | FGG | NM_000509 | chr4 | 155525507-155526047, 155526219-155527855, 155528135-155529616, 155529803- |
| | | | | 155530780, 155530916-155531217, 155531350-155532955, 155533051-155533168, 155533354- |
| | | | | 155533541, 155533588-155533682, |
| 72 | ACCN5 | NM_017419 | chr4 | 156751199-156752974, 156753068-156757839, 156758010-156759963, 156760022- |
| | | | | 156763357, 156763507-156764831, 156764983-156773341, 156773469-156775227, 156775467- |
| | | | | 156784598, 156784907-156787337, |
| 72 | TDO2 | NM_005651 | chr4 | 156824946-156825168, 156825276-156826217, 156826310-156828872, 156828945- |
| | | | | 156830037, 156830167-156831175, 156831364-156832647, 156832757-156835473, 156835587- |
| | | | | 156837005, 156837065-156838545, 156838627-156839299, 156839392-156840987, |
| 73 | LDLRAD3 | NM_174902 | chr11 | 35965695-36057651, 36057800-36103201, 36103329-36119875, 36120012-36248633, 36248981-36250708, |
| 73 | COMMD9 | NM_014186 | chr11 | 36296323-36297685, 36297791-36298637, 36298674-36300025, 36300167-36302260, 36302388-36310910, |
| 73 | COMMD9 | NM_001101653 | chr11 | 36296323-36297685, 36297791-36298637, 36298674-36300025, 36300167-36310910, |
| 73 | C11orf74 | NM_138787 | chr11 | 36616143-36631629, 36631790-36654832, 36654989-36657599, 36657668-36669564, 36669706-36680567, |
| 73 | LRRC4C | NM_020929 | chr11 | 40137885-40313741, |
| 73 | ALKBH3 | NM_139178 | chr11 | 43902730-43904131, 43904282-43904633, 43904739-43905531, 43905568-43908154, 43908204- |
| | | | | 43911273, 43911379-43913589, 43913680-43923064, 43923276-43940586, 43940687-43941466, |
| 73 | AGBL2 | NM_024783 | chr11 | 47681899-47684576, 47684665-47688506, 47688616-47689121, 47689249-47690391, 47690460- |
| | | | | 47698821, 47698954-47701523, 47701626-47703519, 47703648-47707443, 47707602-47711626, 47712411- |
| | | | | 47713653, 47713809-47720996, 47721106-47726093, 47726281-47727071, 47727187-47727414, 47727470- |
| | | | | 47731927, 47732064-47735834, 47735900-47736168, |
| 74 | C10orf18 | NM_017782 | chr10 | 5727139-5754784, 5754882-5759620, 5759714-5762505, 5762632-5762716, 5762738-5762832, 5762934- |
| | | | | 5765638, 5765741-5766391, 5766497-5768846, 5768921-5769022, 5769087-5772448, 5773167- |
| | | | | 5777265, 5777510-5781579, 5782461-5784058, 5784490-5788140, 5791991-5798574, 5798702- |
| | | | | 5799481, 5799637-5800848, 5800956-5803251, 5803435-5804493, 5804610-5804990, |
| 74 | USP6NL | NM_001080491 | chr10 | 11505849-11523767, 11523922-11526968, 11527070-11527166, 11527234-11527814, 11527911- |
| | | | | 11531099, 11531222-11532829, 11532880-11535116, 11535228-11543098, 11543208-11551592, 11551675- |
| | | | | 11560532, 11560574-11567383, 11567468-11569497, 11569567-11574175, |
| 74 | USP6NL | NM_014688 | chr10 | 11505849-11523767, 11523922-11526968, 11527070-11527166, 11527234-11527814, 11527911- |
| | | | | 11531099, 11531222-11532829, 11532880-11535116, 11535228-11543098, 11543208-11551592, 11551675- |
| | | | | 11560532, 11560574-11567383, 11567468-11569497, 11569567-11639628, 11639717-11653440, |
| 74 | MCM10 | NM_182751 | chr10 | 13203607-13206126, 13206210-13212920, 13213264-13214374, 13214481-13214623, 13214766- |
| | | | | 13217508, 13217682-13222440, 13222608-13224931, 13225101-13228162, 13228281-13230879, 13231081- |
| | | | | 13233297, 13233400-13234253, 13234366-13234449, 13234569-13237039, 13237270-13239621, 13239768- |
| | | | | 13240687, 13240808-13243419, 13243535-13246217, 13246365-13251089, 13251134-13251225, |
| 74 | MCM10 | NM_018518 | chr10 | 13203607-13206126, 13206210-13212920, 13213264-13214374, 13214481-13214626, 13214766- |
| | | | | 13217508, 13217682-13222440, 13222608-13224931, 13225101-13228162, 13228281-13230879, 13231081- |
| | | | | 13233297, 13233400-13234253, 13234366-13234449, 13234569-13237039, 13237270-13239621, 13239768- |
| | | | | 13240687, 13240808-13243419, 13243535-13246217, 13246365-13251089, 13251134-13251225, |
| 74 | ABI1 | NM_001178121 | chr10 | 27037675-27040525, 27040713-27047989, 27048165-27054145, 27054248-27057779, 27057922- |
| | | | | 27059172, 27059275-27065992, 27066171-27112065, 27112235-27149674, |
| 74 | ABI1 | NM_001012750 | chr10 | 27037675-27040525, 27040713-27044582, 27044671-27047989, 27048168-27054145, 27054248- |
| | | | | 27057779, 27057922-27059172, 27059275-27060002, 27060019-27065992, 27066171-27112065, 27112235- |
| | | | | 27149674, |
| 74 | ABI1 | NM_005470 | chr10 | 27037675-27040525, 27040713-27044582, 27044671-27047989, 27048168-27052807, 27052890- |
| | | | | 27054145, 27054248-27057779, 27057922-27059172, 27059275-27060002, 27060019-27065992, 27066171- |
| | | | | 27112065, 27112235-27149674, |
| 74 | ABI1 | NM_001178120 | chr10 | 27037675-27040525, 27040713-27047989, 27048165-27054145, 27054248-27057779, 27057922- |
| | | | | 27059172, 27059275-27060002, 27060019-27065992, 27066171-27112065, 27112235-27149674, |
| 74 | ABI1 | NM_001178119 | chr10 | 27037675-27040525, 27040713-27044582, 27044671-27047989, 27048165-27054145, 27054248- |
| | | | | 27057779, 27057922-27059172, 27059275-27060002, 27060019-27065992, 27066171-27112065, 27112235- |
| | | | | 27149674, |
| 74 | ABI1 | NM_001178116 | chr10 | 27037675-27040525, 27040713-27047989, 27048168-27052807, 27052890-27054145, 27054248- |
| | | | | 27057779, 27057922-27059172, 27059275-27060002, 27060019-27065992, 27066171-27112065, 27112235- |
| | | | | 27128660, 27128713-27149674, |
| 74 | ABI1 | NM_001178123 | chr10 | 27037675-27040525, 27040713-27054145, 27054248-27057779, 27057922-27059172, 27059275- |
| | | | | 27060002, 27060019-27065992, 27066171-27112065, 27112235-27149674, |
| 74 | ABI1 | NM_001178122 | chr10 | 27037675-27040525, 27040713-27044582, 27044671-27054145, 27054248-27057779, 27057922- |
| | | | | 27059172, 27059275-27060002, 27060019-27065992, 27066171-27112065, 27112235-27149674, |
| 74 | ABI1 | NM_001012752 | chr10 | 27037675-27040525, 27040713-27044582, 27044671-27047989, 27048168-27054145, 27054248- |
| | | | | 27057779, 27057922-27059172, 27059275-27065992, 27066171-27112065, 27112235-27149674, |
| 74 | ABI1 | NM_001012751 | chr10 | 27037675-27040525, 27040713-27047989, 27048168-27054145, 27054248-27057779, 27057922- |
| | | | | 27059172, 27059275-27060002, 27060019-27065992, 27066171-27112065, 27112235-27149674, |
| 74 | ABI1 | NM_001178124 | chr10 | 27037675-27040525, 27040713-27054145, 27054248-27057779, 27057922-27059172, 27059275- |
| | | | | 27065992, 27066171-27112065, 27112235-27149674, |
| 74 | ABI1 | NM_001178125 | chr10 | 27037675-27040525, 27040713-27054145, 27054248-27057779, 27057922-27059172, 27059275- |
| | | | | 27112065, 27112235-27149674, |
| 74 | LOC729668 | NR_003524 | chr10 | 30653549-30653633, 30653997-30654076, 30654181-30654272, 30654498-30658002, 30658063- |
| | | | | 30658254, 30658355-30658460, 30658549-30662587, 30662783-30663230, |
| 74 | PARD3 | NM_001184786 | chr10 | 34400491-34408539, 34408669-34420389, 34420512-34558583, 34558828-34573061, 34573174- |
| | | | | 34606033, 34606267-34620043, 34620273-34625125, 34625172-34626201, 34626355-34630553, 34630745- |
| | | | | 34636891, 34637044-34648073, 34648246-34648997, 34649188-34663800, 34663931-34666893, 34667035- |
| | | | | 34671466, 34671851-34673055, 34673183-34688256, 34688342-34690752, 34690846-34739243, 34739377- |
| | | | | 34759011, 34759192-34805905, 34806088-34985244, 34985348-35103802, |
| 74 | PARD3 | NM_019619 | chr10 | 34400491-34408539, 34408669-34420389, 34420512-34558583, 34558828-34573061, 34573174- |
| | | | | 34606033, 34606267-34620043, 34620273-34625125, 34625172-34626201, 34626355-34630553, 34630745- |
| | | | | 34636882, 34637044-34648073, 34648246-34648997, 34649188-34661424, 34661465-34663800, 34663931- |
| | | | | 34666893, 34667035-34671466, 34671851-34673055, 34673183-34688256, 34688342-34690752, 34690846- |
| | | | | 34739243, 34739377-34759011, 34759192-34805905, 34806088-34985244, 34985348-35103802, |
| 74 | PARD3 | NM_001184790 | chr10 | 34400491-34408539, 34408669-34420389, 34420512-34558583, 34558828-34573061, 34573174- |
| | | | | 34606033, 34606267-34620043, 34620273-34625125, 34625172-34626291, 34626355-34630553, 34630745- |
| | | | | 34636891, 34637044-34648073, 34648246-34648997, 34649188-34663800, 34663931-34666893, 34667035- |
| | | | | 34671466, 34671851-34673055, 34673183-34688256, 34688342-34690752, 34690846-34759011, 34759192- |
| | | | | 34805905, 34806088-34985244, 34985348-35103802, |
| 74 | PARD3 | NM_001184785 | chr10 | 34400491-34408539, 34408669-34420389, 34420512-34558583, 34558828-34573061, 34573174- |
| | | | | 34606033, 34606267-34620043, 34620273-34625125, 34625172-34626201, 34626355-34630553, 34630745- |
| | | | | 34636891, 34637044-34648073, 34648246-34648997, 34649188-34661424, 34661465-34663800, 34663931- |
| | | | | 34666893, 34667035-34671466, 34671851-34673055, 34673183-34688256, 34688342-34690752, 34690846- |
| | | | | 34739243, 34739377-34759011, 34759192-34805905, 34806088-34985244, 34985348-35103802, |
| 74 | PARD3 | NM_001184788 | chr10 | 34400491-34408539, 34408669-34420389, 34420512-34558583, 34558828-34573061, 34573174- |
| | | | | 34606033, 34606267-34620043, 34620273-34625125, 34625172-34626291, 34626355-34630553, 34630745- |
| | | | | 34636891, 34637044-34648073, 34648246-34648997, 34649188-34663800, 34663931-34666893, 34667035- |
| | | | | 34671466, 34671851-34673055, 34673183-34688256, 34688342-34690752, 34690846-34739243, 34739377- |
| | | | | 34759011, 34759192-34805905, 34806088-34985244, 34985348-35103802, |
| 74 | PARD3 | NM_001184794 | chr10 | 34601311-34606033, 34606267-34620043, 34620273-34625125, 34625172-34626291, 34626355- |
| | | | | 34630553, 34630745-34636891, 34637044-34648073, 34648246-34648997, 34649188-34663800, 34663931- |
| | | | | 34666893, 34667035-34671466, 34671851-34673055, 34673183-34688256, 34688342-34690752, 34690846- |
| | | | | 34739243, 34739377-34759011, 34759192-34805905, 34806088-34985244, 34985348-35103802, |
| 74 | PARD3 | NM_001184791 | chr10 | 34400491-34408539, 34408669-34420389, 34420512-34558583, 34558828-34606033, 34606267- |
| | | | | 34620043, 34620273-34626201, 34626355-34630553, 34630745-34636891, 34637044-34648073, 34648246- |
| | | | | 34648997, 34649188-34663800, 34663931-34666893, 34667035-34671466, 34671851-34673055, 34673183- |
| | | | | 34688256, 34688342-34690752, 34690846-34759011, 34759192-34805905, 34806088-34985244, 34985348- |
| | | | | 35103802, |
| 74 | PARD3 | NM_001184792 | chr10 | 34601311-34606033, 34606267-34620043, 34620273-34625125, 34625172-34626201, 34626355- |
| | | | | 34630553, 34630745-34636891, 34637044-34648073, 34648246-34648997, 34649188-34661424, 34661465- |
| | | | | 34663800, 34663931-34666893, 34667035-34671466, 34671851-34673055, 34673183-34688256, 34688342- |
| | | | | 34690752, 34690846-34739243, 34739377-34759011, 34759192-34805905, 34806088-34985244, 34985348- |
| | | | | 35103802, |
| 74 | PARD3 | NM_001184787 | chr10 | 34400491-34408539, 34408669-34420389, 34420512-34558583, 34558828-34606033, 34606267- |
| | | | | 34620043, 34620273-34625125, 34625172-34626201, 34626355-34630553, 34630745-34636882, 34637044- |
| | | | | 34648073, 34648246-34648997, 34649188-34661424, 34661465-34663800, 34663931-34666893, 34667035- |
| | | | | 34671466, 34671851-34673055, 34673183-34688256, 34688342-34690752, 34690846-34739243, 34739377- |
| | | | | 34759011, 34759192-34805905, 34806088-34985244, 34985348-35103802, |
| 74 | PARD3 | NM_001184789 | chr10 | 34400491-34408539, 34408669-34420389, 34420512-34558583, 34558828-34606033, 34606267- |
| | | | | 34620043, 34620273-34625125, 34625172-34626291, 34626355-34630553, 34630745-34636891, 34637044- |
| | | | | 34648073, 34648246-34648997, 34649188-34663800, 34663931-34666893, 34667035-34671466, 34671851- |
| | | | | 34673055, 34673183-34688256, 34688342-34690752, 34690846-34739243, 34739377-34759011, 34759192- |
| | | | | 34805905, 34806088-34985244, 34985348-35103802, |
| 74 | PARD3 | NM_001184793 | chr10 | 34601311-34606033, 34606267-34620043, 34620273-34625125, 34625169-34626291, 34626355- |
| | | | | 34630553, 34630745-34636891, 34637044-34648073, 34648246-34648997, 34649188-34661424, 34661465- |
| | | | | 34663800, 34663931-34666893, 34667035-34671466, 34671851-34673055, 34673183-34688256, 34688342- |
| | | | | 34690752, 34690846-34739243, 34739377-34759011, 34759192-34805905, 34806088-34985244, 34985348- |
| | | | | 35103802, |
| 75 | C19orf20 | NM_033513 | chr19 | 507845-518887, |
| 75 | RNF126 | NM_194460 | chr19 | 648278-648370, 648488-648880, 648976-649677, 649749-650232, 650297-651609, 651856-652231, 652297- |
| | | | | 652824, 652885-663045, |
| 75 | FSTL3 | NM_005860 | chr19 | 676527-677790, 677978-680272, 680490-681331, 681561-681648, |
| 75 | PALM | NM_002579 | chr19 | 709152-726136, 726190-727006, 727089-727562, 727695-731093, 731246-734171, 734195-736017, 736079- |
| | | | | 740350, 740484-746283, |
| 75 | PALM | NM_001040134 | chr19 | 709152-726136, 726190-727006, 727089-727562, 727695-731093, 731246-734171, 734195-736017, 736079- |
| | | | | 746283, |
| 75 | MED16 | NM_005481 | chr19 | 868252-868414, 868500-868861, 868947-871035, 871254-871924, 872119-873447, 873583-875242, 875455- |
| | | | | 876972, 877181-879935, 880149-881557, 881715-884901, 885009-885768, 886202-889636, 889808- |
| | | | | 890135, 890245-890961, 891150-893084, |
| 75 | STK11 | NM_000455 | chr19 | 1207203-1218414, 1218500-1219321, 1219413-1220370, 1220505-1220578, 1220717-1221210, 1221340- |
| | | | | 1221946, 1222006-1222982, 1223172-1226451, 1226663-1227590, |
| 76 | GTSE1 | NM_016426 | chr22 | 46692829-46693275, 46693377-46704045, 46704105-46704214, 46704841-46708036, 46708203- |
| | | | | 46709785, 46709911-46711927, 46712310-46719085, 46719160-46722331, 46722552-46724583, 46724787- |
| | | | | 46725253, 46725465-46725942, |
| 76 | BRD1 | NM_014577 | chr22 | 50168065-50169237, 50169394-50169696, 50169808-50170680, 50170810-50171324, 50171462- |
| | | | | 50181036, 50181143-50187680, 50187943-50191451, 50191766-50192204, 50192335-50192634, 50192768- |
| | | | | 50197850, 50198009-50216597, |
| 76 | ZBED4 | NM_014838 | chr22 | 50247638-50276980, |
| 76 | TRABD | NM_025204 | chr22 | 50624431-50631454, 50631523-50632003, 50632084-50632767, 50632936-50633327, 50633470- |
| | | | | 50635668, 50635781-50635876, 50636018-50636250, 50636425-50636502, 50636616-50636772, |
| 76 | SELO | NM_031454 | chr22 | 50640020-50644744, 50644950-50646963, 50647146-50648608, 50648741-50649058, 50649341- |
| | | | | 50654144, 50654297-50655118, 50655306-50655399, 50655558-50655632, |
| 76 | PPP6R2 | NM_014678 | chr22 | 50781980-50810447, 50810580-50832320, 50832565-50845116, 50845305-50852979, 50853119- |
| | | | | 50854519, 50854587-50857294, 50857409-50857776, 50857892-50860681, 50860810-50861889, 50862044- |
| | | | | 50869600, 50869812-50870692, 50870759-50873393, 50873511-50874794, 50874882-50875933, 50876033- |
| | | | | 50876270, 50876330-50876601, 50876728-50877026, 50877192-50878128, 50878287-50878388, 50878479- |
| | | | | 50879249, 50879456-50882293, 50882547-50882626, |
| 76 | ADM2 | NM_024866 | chr22 | 50920387-50920994, |
| 76 | RPL23AP82 | NR_026982 | chr22 | 51222501-51223599, 51223722-51237081, |
| 76 | RPL23AP82 | NR_026981 | chr22 | 51195755-51221546, 51221723-51223599, 51223722-51237081, |
| 77 | DIXDC1 | NM_001037954 | chr11 | 111808284-111835271, 111835403-111839235, 111839363-111844745, 111844979- |
| | | | | 111845598, 111845708-111851460, 111851575-111853062, 111853212-111855954, 111856046- |
| | | | | 111857593, 111857649-111859727, 111859780-111863137, 111863244-111863673, 111863729- |
| | | | | 111864218, 111864313-111864391, 111864465-111865741, 111865833-111866125, 111866289- |
| | | | | 111866802, 111866871-111887413, 111887521-111888499, 111888610-111889678, |
| 77 | DIXDC1 | NM_033425 | chr11 | 111848520-111851460, 111851575-111853062, 111853212-111855954, 111856046- |
| | | | | 111857593, 111857649-111859727, 111859780-111863137, 111863244-111863673, 111863729- |
| | | | | 111864218, 111864313-111864391, 111864465-111865741, 111865833-111866125, 111866289- |
| | | | | 111866802, 111866871-111887413, 111887521-111888499, 111888610-111889678, |
| 77 | PIH1D2 | NM_001082619 | chr11 | 111934946-111941158, 111941426-111941760, 111942008-111942357, 111942483- |
| | | | | 111943720, 111943930-111944702, |
| 77 | PIH1D2 | NM_138789 | chr11 | 111938730-111941158, 111941426-111941760, 111942008-111942357, 111942483- |
| | | | | 111943720, 111943930-111944702, |
| 77 | C11orf34 | NM_001145024 | chr11 | 112119698-112123069, 112123133-112126109, 112126313-112131127, |
| 77 | TTC12 | NM_017868 | chr11 | 113185341-113186963, 113187038-113194008, 113194174-113194714, 113194738- |
| | | | | 113195325, 113195405-113196244, 113196368-113200640, 113200702-113205686, 113205760- |
| | | | | 113209494, 113209557-113210006, 113210197-113211384, 113211456-113212557, 113212648- |
| | | | | 113214992, 113215163-113220793, 113220888-113221959, 113222022-113222790, 113222930- |
| | | | | 113230083, 113230184-113230660, 113230731-113233121, 113233225-113234548, 113234650- |
| | | | | 113235555, 113235783-113236945, |
| 77 | ANKK1 | NM_178510 | chr11 | 113258792-113264201, 113264498-113265649, 113265803-113266079, 113266131- |
| | | | | 113266787, 113266945-113267944, 113268065-113268700, 113268739-113269684, |
| 77 | USP28 | NM_020886 | chr11 | 113670138-113672203, 113672401-113673878, 113674004-113674518, 113674600- |
| | | | | 113675393, 113675474-113675588, 113675769-113677209, 113677307-113679018, 113679160- |
| | | | | 113679783, 113679977-113682996, 113683227-113684605, 113684678-113685894, 113686105- |
| | | | | 113688378, 113688560-113694325, 113694423-113697953, 113698083-113699917, 113700068- |
| | | | | 113701587, 113701666-113702640, 113702716-113704140, 113704280-113704969, 113705058- |
| | | | | 113711318, 113711480-113712383, 113712491-113723221, 113723356-113724978, 113725058- |
| | | | | 113746165, |
| 77 | RNF214 | NM_207343 | chr11 | 117103443-117104981, 117105096-117109315, 117109828-117110515, 117110577- |
| | | | | 117115159, 117115302-117117523, 117117665-117150622, 117150721-117150885, 117150976- |
| | | | | 117152028, 117152121-117152314, 117152415-117152608, 117152987-117153118, 117153253- |
| | | | | 117153460, 117153567-117153829, 117153927-117155800, |
| 77 | RNF214 | NM_001077239 | chr11 | 117103492-117104981, 117105096-117109315, 117109828-117110515, 117110577- |
| | | | | 117115159, 117115302-117117523, 117117665-117150622, 117150721-117150885, 117150976- |
| | | | | 117152028, 117152121-117152314, 117152415-117152608, 117152987-117153118, 117153253- |
| | | | | 117153460, 117153567-117153829, 117153927-117155800, |
| 78 | SPATA5L1 | NM_024063 | chr15 | 45695717-45697516, 45697704-45702568, 45702729-45706767, 45706979-45707783, 45707978- |
| | | | | 45709465, 45709598-45710753, 45710881-45713239, |
| 78 | SPATA5L1 | NR_027635 | chr15 | 45695717-45697516, 45697704-45702568, 45702729-45706767, 45706979-45707783, 45708008- |
| | | | | 45709465, 45709598-45710753, 45710881-45713239, |
| 78 | CTXN2 | NM_001145668 | chr15 | 48484051-48493439, |
| 78 | SLC12A1 | NM_000338 | chr15 | 48498528-48499729, 48500337-48512829, 48512963-48513116, 48513194-48518671, 48518769- |
| | | | | 48521384, 48521526-48522588, 48522701-48524922, 48525036-48527072, 48527202-48533710, 48533797- |
| | | | | 48536948, 48537102-48539104, 48539214-48539532, 48539658-48541770, 48541874-48543810, 48543968- |
| | | | | 48548006, 48548108-48551395, 48551509-48559756, 48559899-48561853, 48561962-48566766, 48566851- |
| | | | | 48577301, 48577447-48580238, 48580372-48580600, 48580714-48583973, 48584062-48591335, 48591473- |
| | | | | 48593510, 48593580-48594945, |
| 78 | SLC12A1 | NM_001184832 | chr15 | 48498528-48499729, 48500337-48512829, 48512963-48513116, 48513194-48519274, 48519372- |
| | | | | 48521384, 48521526-48522588, 48522701-48524922, 48525036-48527072, 48527202-48533710, 48533797- |
| | | | | 48536948, 48537102-48539104, 48539214-48539532, 48539658-48541770, 48541874-48543810, 48543968- |
| | | | | 48548006, 48548108-48551395, 48551509-48559756, 48559899-48561853, 48561962-48566766, 48566851- |
| | | | | 48577301, 48577447-48580238, 48580372-48580600, 48580714-48583973, 48584062-48591335, 48591473- |
| | | | | 48593510, 48593580-48594945, |
| 78 | CEP152 | NM_001194998 | chr15 | 49031486-49033796, 49033902-49034142, 49034298-49036435, 49036541-49037091, 49037190- |
| | | | | 49040638, 49040808-49044544, 49044667-49048098, 49048751-49052330, 49052464-49054586, 49054870- |
| | | | | 49059255, 49059390-49059530, 49059661-49060414, 49060526-49061151, 49061279-49064682, 49064889- |
| | | | | 49073391, 49073557-49074334, 49074428-49076168, 49076318-49080996, 49081199-49083432, 49083574- |
| | | | | 49085516, 49085659-49088205, 49088358-49089496, 49089777-49089856, 49089928-49090143, 49090249- |
| | | | | 49097758, 49097854-49103156, |
| 78 | CEP152 | NM_014985 | chr15 | 49031486-49033796, 49033902-49034142, 49034298-49036435, 49036541-49037091, 49037190- |
| | | | | 49044544, 49044667-49048098, 49048751-49052330, 49052464-49054586, 49054870-49059255, 49059390- |
| | | | | 49059530, 49059661-49060414, 49060526-49061151, 49061279-49064682, 49064889-49073391, 49073557- |
| | | | | 49074334, 49074428-49076168, 49076318-49080996, 49081199-49083432, 49083574-49085516, 49085659- |
| | | | | 49088205, 49088358-49089496, 49089777-49089856, 49089928-49090143, 49090249-49097758, 49097854- |
| | | | | 49103156, |
| 78 | SECISBP2L | NM_014701 | chr15 | 49285124-49288562, 49288784-49292028, 49292185-49293072, 49293295-49301411, 49301576- |
| | | | | 49303926, 49304061-49304843, 49305015-49308755, 49308899-49309043, 49309213-49309743, 49309826- |
| | | | | 49319560, 49319678-49320339, 49320366-49320648, 49320880-49325160, 49325298-49327529, 49327856- |
| | | | | 49329786, 49329967-49338471, |
| 78 | SECISBP2L | NM_001193489 | chr15 | 49285124-49288562, 49288784-49292028, 49292185-49293072, 49293295-49301411, 49301576- |
| | | | | 49303926, 49304061-49304843, 49305015-49308755, 49308899-49309043, 49309213-49309743, 49309826- |
| | | | | 49311613, 49311750-49319560, 49319678-49320339, 49320366-49320648, 49320880-49325160, 49325298- |
| | | | | 49327529, 49327856-49329786, 49329967-49338471, |
| 78 | GALK2 | NM_002044 | chr15 | 49462573-49493357, 49493448-49509385, 49509511-49528046, 49528139-49531416, 49531565- |
| | | | | 49574182, 49574283-49575761, 49575916-49584522, 49584735-49611799, 49612003-49620147, |
| 78 | GALK2 | NM_001001556 | chr15 | 49448214-49493357, 49493448-49509385, 49509511-49528046, 49528139-49531416, 49531565- |
| | | | | 49574182, 49574283-49575761, 49575916-49584522, 49584735-49611799, 49612003-49620147, |
| 78 | GABPB1 | NM_016654 | chr15 | 50570982-50578263, 50578381-50581678, 50581866-50592984, 50593100-50593452, 50593566- |
| | | | | 50595124, 50595321-50596161, 50596331-50601886, 50601996-50647180, |
| 78 | GABPB1 | NM_181427 | chr15 | 50578381-50581678, 50581866-50592984, 50593100-50593452, 50593566-50595124, 50595321- |
| | | | | 50596161, 50596331-50601886, 50601996-50638567, 50638593-50647180, |
| 78 | GABPB1 | NM_005254 | chr15 | 50570982-50578263, 50578381-50581678, 50581866-50592984, 50593100-50593416, 50593566- |
| | | | | 50595124, 50595321-50596161, 50596331-50601886, 50601996-50647180, |
| 78 | GABPB1 | NM_002041 | chr15 | 50578381-50581678, 50581866-50592984, 50593100-50593416, 50593566-50595124, 50595321- |
| | | | | 50596161, 50596331-50601886, 50601996-50647180, |
| 78 | GABPB1 | NM_016655 | chr15 | 50578381-50581678, 50581866-50592984, 50593100-50593452, 50593566-50595124, 50595321- |
| | | | | 50596161, 50596331-50601886, 50601996-50647180, |
| 78 | USP8 | NM_005154 | chr15 | 50716712-50731204, 50731375-50733544, 50733691-50741595, 50741683-50751195, 50751360- |
| | | | | 50754475, 50754520-50757242, 50757389-50763828, 50763993-50769044, 50769191-50769471, 50769697- |
| | | | | 50773676, 50774263-50776470, 50776559-50781996, 50782079-50782458, 50782723-50784896, 50785111- |
| | | | | 50786265, 50786478-50788043, 50788282-50789284, 50789429-50790791, 50790926-50791098, |
| 78 | USP8 | NM_001128611 | chr15 | 50716852-50724533, 50724697-50731204, 50731375-50733544, 50733691-50741595, 50741683- |
| | | | | 50751195, 50751360-50754475, 50754520-50757242, 50757389-50763828, 50763993-50769044, 50769191- |
| | | | | 50769471, 50769697-50773676, 50774263-50776470, 50776559-50781996, 50782079-50782458, 50782723- |
| | | | | 50784896, 50785111-50786265, 50786478-50788043, 50788282-50789284, 50789429-50790791, 50790926- |
| | | | | 50791098, |
| 78 | USP8 | NM_001128610 | chr15 | 50716852-50731204, 50731375-50733544, 50733691-50741595, 50741683-50751195, 50751360- |
| | | | | 50754475, 50754520-50757242, 50757389-50763828, 50763993-50769044, 50769191-50769471, 50769697- |
| | | | | 50773676, 50774263-50776470, 50776559-50781996, 50782079-50782458, 50782723-50784896, 50785111- |
| | | | | 50786265, 50786478-50788043, 50788282-50789284, 50789429-50790791, 50790926-50791098, |
| 78 | TRPM7 | NM_017672 | chr15 | 50854006-50862082, 50862191-50862299, 50862353-50866469, 50866677-50866832, 50866917- |
| | | | | 50867047, 50867333-50867919, 50867987-50868064, 50868117-50870834, 50870862-50873069, 50873106- |
| | | | | 50875284, 50875357-50878587, 50878686-50881787, 50881854-50884106, 50884824-50885812, 50885947- |
| | | | | 50886624, 50886811-50888450, 50888579-50891317, 50891494-50897061, 50897342-50899395, 50899526- |
| | | | | 50901776, 50901922-50902001, 50902159-50903288, 50903519-50904744, 50905027-50905902, 50906039- |
| | | | | 50906318, 50906461-50911940, 50911996-50916361, 50916498-50920344, 50920447-50923612, 50923687- |
| | | | | 50925064, 50925190-50926577, 50926754-50929617, 50929791-50931619, 50931746-50935535, 50935751- |
| | | | | 50940882, 50941083-50949976, 50950017-50955162, 50955244-50978726, |
| 78 | SPPL2A | NM_032802 | chr15 | 51000072-51012135, 51012298-51014319, 51014399-51017416, 51017521-51018516, 51018575- |
| | | | | 51023160, 51023237-51024798, 51024882-51028296, 51028400-51028839, 51028938-51031875, 51032026- |
| | | | | 51039690, 51039826-51040308, 51040400-51040883, 51041068-51041831, 51041944-51057663, |
| 78 | AP4E1 | NM_007347 | chr15 | 51201126-51204273, 51204347-51207643, 51207769-51216126, 51216202-51217293, 51217417- |
| | | | | 51221204, 51221366-51223000, 51223169-51226819, 51226895-51233637, 51233762-51233861, 51233973- |
| | | | | 51240215, 51240357-51242021, 51242136-51242246, 51242367-51250687, 51250992-51260458, 51260575- |
| | | | | 51276217, 51276343-51285565, 51285823-51289521, 51290081-51291267, 51291460-51293221, 51293381- |
| | | | | 51294697, |
| 78 | TNFAIP8L3 | NM_207381 | chr15 | 51350641-51386739, 51386885-51397200, |
| 78 | CYP19A1 | NM_000103 | chr15 | 51503254-51504515, 51504759-51507265, 51507430-51507898, 51508015-51510736, 51510853- |
| | | | | 51514544, 51514723-51519974, 51520131-51529054, 51529207-51534963, 51535148-51630690, |
| 78 | CYP19A1 | NM_031226 | chr15 | 51503254-51504515, 51504759-51507265, 51507430-51507898, 51508015-51510736, 51510853- |
| | | | | 51514544, 51514723-51519974, 51520131-51529054, 51529207-51534963, 51535148-51616011, 51616122- |
| | | | | 51630690, |
| 78 | DMXL2 | NM_001174117 | chr15 | 51741391-51742324, 51742544-51743840, 51743895-51745745, 51745839-51747346, 51747426- |
| | | | | 51748261, 51748391-51748504, 51748567-51749523, 51749664-51750701, 51750825-51750904, 51750993- |
| | | | | 51755574, 51755694-51756870, 51757074-51757761, 51757849-51758379, 51758509-51763418, 51763599- |
| | | | | 51766539, 51766790-51768784, 51768914-51770466, 51770545-51772143, 51772337-51772737, 51773821- |
| | | | | 51778268, 51778535-51780149, 51780317-51780743, 51780843-51783773, 51783945-51787219, 51787332- |
| | | | | 51799329, 51799451-51806637, 51806757-51809273, 51809365-51827858, 51827982-51828361, 51829060- |
| | | | | 51829683, 51829957-51830408, 51830650-51834528, 51834705-51837778, 51837964-51839425, 51839606- |
| | | | | 51855576, 51855645-51856320, 51856458-51857283, 51857364-51860682, 51860756-51868251, 51868379- |
| | | | | 51914654, |
| 78 | DMXL2 | NM_015263 | chr15 | 51741391-51742324, 51742544-51743840, 51743895-51745745, 51745839-51747346, 51747426- |
| | | | | 51748261, 51748391-51748504, 51748567-51749523, 51749664-51750701, 51750825-51750904, 51750993- |
| | | | | 51755574, 51755694-51756870, 51757074-51757761, 51757849-51758379, 51758509-51763418, 51763599- |
| | | | | 51766539, 51766790-51768784, 51768914-51770466, 51770545-51772143, 51772337-51772737, 51773821- |
| | | | | 51778268, 51778535-51780149, 51780317-51780743, 51780843-51783773, 51783945-51787219, 51787332- |
| | | | | 51790747, 51792429-51795001, 51795231-51799329, 51799451-51806637, 51806757-51809273, 51809365- |
| | | | | 51827858, 51827982-51828361, 51829060-51829683, 51829957-51830408, 51830650-51834528, 51834705- |
| | | | | 51837778, 51837964-51839425, 51839606-51855576, 51855645-51856320, 51856458-51857283, 51857364- |
| | | | | 51860682, 51860756-51868251, 51868379-51914654, |
| 78 | DMXL2 | NM_001174116 | chr15 | 51741391-51742324, 51742544-51743840, 51743895-51745745, 51745839-51747346, 51747426- |
| | | | | 51748261, 51748391-51748504, 51748567-51749523, 51749664-51750701, 51750825-51750904, 51750993- |
| | | | | 51755574, 51755694-51756870, 51757074-51757761, 51757849-51758379, 51758509-51763418, 51763599- |
| | | | | 51766539, 51766790-51768784, 51768917-51770466, 51770545-51772143, 51772337-51772737, 51773821- |
| | | | | 51778268, 51778535-51780149, 51780317-51780743, 51780843-51783773, 51783945-51787219, 51787332- |
| | | | | 51790747, 51792429-51795001, 51795231-51799329, 51799451-51806637, 51806757-51809273, 51809365- |
| | | | | 51827858, 51827982-51828361, 51829060-51829683, 51829957-51830408, 51830650-51834528, 51834705- |
| | | | | 51837778, 51837964-51839425, 51839606-51855576, 51855645-51856320, 51856458-51857283, 51857364- |
| | | | | 51860682, 51860756-51868251, 51868379-51914654, |
| 78 | TMOD2 | NM_014548 | chr15 | 52043910-52058568, 52058765-52060457, 52060616-52065907, 52066032-52069127, 52069216- |
| | | | | 52073239, 52073372-52074916, 52075026-52090392, 52090538-52098572, 52098719-52100615, |
| 78 | TMOD2 | NM_001142885 | chr15 | 52043910-52058568, 52058765-52060457, 52060616-52065907, 52066032-52069127, 52069216- |
| | | | | 52073239, 52073372-52090392, 52090538-52098572, 52098719-52100615, |
| 78 | TMOD3 | NM_014547 | chr15 | 52122034-52155006, 52155208-52161412, 52161571-52179784, 52179909-52181251, 52181343- |
| | | | | 52186010, 52186143-52188614, 52188724-52192350, 52192496-52194087, 52194234-52200971, |
| 78 | MAPK6 | NM_002748 | chr15 | 52311574-52338025, 52339213-52342188, 52342335-52350828, 52350995-52353494, 52353698-52356097, |
| 78 | ARPP19 | NM_006628 | chr15 | 52844302-52849295, 52849420-52861043, |
| 79 | AP1G1 | NM_001128 | chr16 | 71767063-71768510, 71768611-71772843, 71773006-71773135, 71773245-71779045, 71779174- |
| | | | | 71779374, 71779518-71780489, 71780596-71782152, 71782282-71783795, 71783887-71784111, 71784236- |
| | | | | 71787734, 71787791-71789920, 71790063-71792709, 71792825-71795413, 71795471-71798251, 71798352- |
| | | | | 71798547, 71798630-71799390, 71799488-71803524, 71803603-71805057, 71805156-71807122, 71807266- |
| | | | | 71808369, 71808496-71823180, 71823386-71842664, |
| 79 | AP1G1 | NM_001030007 | chr16 | 71767063-71768510, 71768611-71772843, 71773006-71773135, 71773245-71779045, 71779174- |
| | | | | 71779374, 71779518-71780489, 71780596-71782152, 71782282-71783795, 71783887-71784111, 71784236- |
| | | | | 71787734, 71787791-71789920, 71790063-71792709, 71792825-71795413, 71795471-71798251, 71798352- |
| | | | | 71798547, 71798630-71799390, 71799490-71800340, 71800349-71803524, 71803603-71805057, 71805156- |
| | | | | 71807122, 71807266-71808369, 71808496-71823180, 71823386-71842664, |
| 79 | KIAA0174 | NM_014761 | chr16 | 71929493-71949527, 71949632-71950363, 71950546-71950948, 71951038-71954640, 71954726- |
| | | | | 71955244, 71955357-71956375, 71956584-71957189, 71957284-71958674, 71958721-71961515, |
| 79 | PSMD7 | NM_002811 | chr16 | 74330887-74334011, 74334105-74334948, 74335043-74335451, 74335551-74336108, 74336191- |
| | | | | 74338199, 74338293-74339185, |
| 79 | GLG1 | NM_012201 | chr16 | 74486030-74487067, 74487233-74490545, 74490654-74491770, 74491893-74493578, 74493688- |
| | | | | 74495990, 74496094-74496384, 74496529-74497252, 74497378-74499572, 74499712-74501652, 74501747- |
| | | | | 74502842, 74502962-74503881, 74503972-74505069, 74505185-74506245, 74506310-74508442, 74508531- |
| | | | | 74511292, 74511432-74514137, 74514293-74516919, 74517023-74519692, 74519816-74524897, 74525114- |
| | | | | 74526853, 74527039-74528656, 74528730-74530337, 74530543-74537427, 74537645-74542735, 74542824- |
| | | | | 74566017, 74566052-74640553, |
| 79 | GLG1 | NR_027264 | chr16 | 74487233-74490545, 74490654-74491770, 74491893-74493578, 74493688-74495990, 74496094- |
| | | | | 74496384, 74496529-74497252, 74497378-74499572, 74499712-74501652, 74501747-74502842, 74502962- |
| | | | | 74503881, 74503972-74505069, 74505185-74506245, 74506310-74506422, 74506533-74508442, 74508531- |
| | | | | 74511292, 74511432-74514137, 74514293-74516919, 74517023-74519692, 74519816-74524897, 74525114- |
| | | | | 74526853, 74527039-74528656, 74528730-74530337, 74530543-74537427, 74537645-74542735, 74542824- |
| | | | | 74566017, 74566052-74640553, |
| 79 | GLG1 | NR_027265 | chr16 | 74487233-74490545, 74490654-74491770, 74491893-74493578, 74493688-74495990, 74496094- |
| | | | | 74496384, 74496529-74497252, 74497378-74499572, 74499712-74501652, 74501747-74502842, 74502962- |
| | | | | 74503881, 74503972-74505069, 74505185-74506245, 74506310-74508442, 74508531-74511292, 74511432- |
| | | | | 74514137, 74514173-74530525, 74530543-74537427, 74537645-74542735, 74542824-74566017, 74566052- |
| | | | | 74640553, |
| 79 | GLG1 | NM_001145667 | chr16 | 74487233-74490545, 74490654-74491770, 74491893-74493578, 74493688-74495990, 74496094- |
| | | | | 74496384, 74496529-74497252, 74497378-74499572, 74499712-74501652, 74501747-74502842, 74502962- |
| | | | | 74503881, 74503972-74505069, 74505185-74506245, 74506310-74508442, 74508531-74511292, 74511432- |
| | | | | 74514137, 74514293-74516919, 74517023-74519692, 74519816-74524897, 74525114-74526853, 74527039- |
| | | | | 74528656, 74528730-74530337, 74530543-74537427, 74537645-74542735, 74542824-74566017, 74566052- |
| | | | | 74640553, |
| 79 | GLG1 | NM_001145666 | chr16 | 74486030-74487067, 74487233-74490545, 74490654-74491770, 74491893-74493578, 74493688- |
| | | | | 74495990, 74496094-74496384, 74496529-74497252, 74497378-74499572, 74499712-74501652, 74501747- |
| | | | | 74502842, 74502962-74503881, 74503972-74505069, 74505185-74506245, 74506310-74508442, 74508531- |
| | | | | 74511292, 74511432-74514137, 74514293-74516919, 74517023-74519692, 74519816-74524897, 74525114- |
| | | | | 74526853, 74527039-74528656, 74528730-74530337, 74530543-74537427, 74537645-74542735, 74542824- |
| | | | | 74640553, |
| 79 | RFWD3 | NM_018124 | chr16 | 74657970-74660239, 74660453-74662348, 74662565-74664677, 74664856-74666419, 74666572- |
| | | | | 74670242, 74670476-74671752, 74671869-74678258, 74678352-74678437, 74678634-74683028, 74683101- |
| | | | | 74685816, 74686021-74694828, 74695350-74700682, |
| 79 | CFDP1 | NM_006324 | chr16 | 75327941-75338920, 75339081-75428986, 75429108-75445721, 75445851-75446431, 75446653- |
| | | | | 75448474, 75448594-75467185, |

TABLE 6A
75 Mapped Strings from Whole Gene (Exon + Intron) Analysis of the Providence Dataset.

| | Strings Based on Identified Whole Genes | | | | | | | | |
|---|---|---|---|---|---|---|---|---|---|
| | | | | | | | | | RM- |
| | | | | | | | | | Corrected |
| | | | | Exemplary | Exemplary | | | | Absolute |
| | | Chromo- | | Start | End | # | Direction | | Standardized |
| | | some | | Location of | Location of | Genes | of | | Hazard Ratio |
| String | | (23 = X, | | First Gene | Last Gene in | in | Associ- | | (95% Conf. |
| No. | Genes in Run | 24 = Y) | Arm | in Run | Run | Run | ation | p-value | Int.) |
| 1 | SLCO6A1 EFNA5 ALDH7A1 PRRC1 | 5 | q | 101,707,651 | 140,998,622 | 14 | 1 | 2.25E−11 | 1.82 (1.53, |
| | VDAC1 SEC24A TRPC7 KIF20A CDC25C | | | | | | | | 2.17) |
| | HSPA9 NRG2 PCDHGA6 PCDHGB4 | | | | | | | | |
| | DIAPH1 | | | | | | | | |
| 2 | CYP4F8 UCA1 EPS15L1 SIN3B | 19 | p | 15,726,028 | 21,721,079 | 12 | −1 | 8.36E−11 | 1.78 (1.49, |
| | TMEM221 LOC284440 ZNF682 | | | | | | | | 2.11) |
| | ZNF626 ZNF85 ZNF708 ZNF738 | | | | | | | | |
| | ZNF429 | | | | | | | | |
| 3 | DIO1 HSPB11 LRRC42 FAM151A | 1 | p | 54,359,860 | 84,964,033 | 17 | −1 | 3.45E−10 | 1.77 (1.48, |
| | C1orf168 DAB1 OMA1 WDR78 | | | | | | | | 2.11) |
| | SLC35D1 HHLA3 PTGER3 NEGR1 | | | | | | | | |
| | C1orf173 CRYZ TYW3 FAM73A RPF1 | | | | | | | | |
| 4 | MICAL3 GGT3P DGCR6 UFD1L CDC45 | 22 | q | 18,270,415 | 25,055,114 | 10 | 1 | 3.83E−12 | 1.77 (1.50, |
| | PRAME CHCHD10 GSTTP1 GGT1 | | | | | | | | 2.07) |
| | POM121L10P | | | | | | | | |
| 5 | ZNF37BP ZNF487P ZNF239 | 10 | q | 43,008,960 | 46,970,601 | 6 | −1 | 5.10E−11 | 1.75 (1.48, |
| | LOC283033 ANUBL1 SYT15 | | | | | | | | 2.06) |
| 6 | PMAIP1 BCL2 SERPINB8 FBXO15 | 18 | q | 57,567,191 | 77,929,616 | 8 | −1 | 4.91E−10 | 1.73 (1.46, |
| | LOC400657 ZNF516 LOC284276 | | | | | | | | 2.06) |
| | LOC100130522 | | | | | | | | |
| 7 | ERRFI1 ENO1 KIF1B PGD SRM NPPB | 1 | p | 8,071,778 | 12,677,820 | 7 | 1 | 1.45E−10 | 1.73 (1.46, |
| | DHRS3 | | | | | | | | 2.04) |
| 8 | HINFP CCDC153 GRIK4 TBCEL TECTA | 11 | q | 118,992,232 | 130,298,539 | 10 | −1 | 2.18E−10 | 1.72 (1.45, |
| | VWA5A SPA17 SLC37A2 KCNJ5 | | | | | | | | 2.03) |
| | ADAMTS8 | | | | | | | | |
| 9 | C9orf64 LOC389765 NAA35 CDK20 | 9 | q | 86,553,226 | 100,684,852 | 31 | −1 | 5.24E−10 | 1.72 (1.45, |
| | SPIN1 NXNL2 SECISBP2 NOL8 CENPP | | | | | | | | 2.04) |
| | ECM2 BICD2 ANKRD19 ZNF484 FGD3 | | | | | | | | |
| | SUSD3 C9orf89 NINJ1 FAM120AOS | | | | | | | | |
| | FAM120A PHF2 FBP1 C9orf130 | | | | | | | | |
| | C9orf102 SLC35D2 ZNF510 ZNF782 | | | | | | | | |
| | LOC57653 TSTD2 NCBP1 XPA | | | | | | | | |
| | C9orf156 | | | | | | | | |
| 10 | NLRP8 LOC386758 ZFP28 ZNF71 | 19 | q | 56,459,197 | 58,951,589 | 8 | −1 | 4.43E−09 | 1.70 (1.43, |
| | ZNF835 ZNF211 C19orf18 ZNF132 | | | | | | | | 2.03) |
| 11 | TCERG1 SPINK5 IRGM GLRA1 LARP1 | 5 | q | 145,826,872 | 160,365,633 | 8 | 1 | 2.03E−10 | 1.69 (1.44, |
| | CLINT1 RNF145 LOC285629 | | | | | | | | 1.99) |
| 12 | NOSTRIN LRP2 BBS5 KBTBD10 PPIG | 2 | q | 169,643,048 | 172,750,816 | 12 | −1 | 1.38E−09 | 1.69 (1.42, |
| | C2orf77 PHOSPHO2 KLHL23 DCAF17 | | | | | | | | 2.00) |
| | CYBRD1 DYNC1I2 SLC25A12 | | | | | | | | |
| 13 | RRP8 DCHS1 ZNF214 SYT9 TRIM66 | 11 | p | 6,621,143 | 9,113,150 | 7 | −1 | 4.57E−09 | 1.67 (1.41, |
| | NRIP3 SCUBE2 | | | | | | | | 1.99) |
| 14 | RAD52 DCP1B PARP11 C12orf5 | 12 | p | 1,021,254 | 16,190,315 | 19 | −1 | 8.41E−09 | 1.67 (1.40, |
| | VAMP1 ACRBP C12orf53 LRRC23 | | | | | | | | 1.99) |
| | ATN1 FOXJ2 CLEC4A FAM66C | | | | | | | | |
| | FAM90A1 CREBL2 DDX47 HEBP1 GSG1 | | | | | | | | |
| | RERG DERA | | | | | | | | |
| 15 | FRMD8 CFL1 YIF1A TMEM151A | 11 | q | 65,154,040 | 71,159,477 | 10 | 1 | 2.18E−09 | 1.66 (1.41, |
| | NDUFV1 NUDT8 TBX10 CHKA | | | | | | | | 1.96) |
| | C11orf24 DHCR7 | | | | | | | | |
| 16 | ASTN2 TRIM32 MEGF9 C5 RAB14 | 9 | q | 119,187,503 | 138,391,761 | 35 | −1 | 1.54E−08 | 1.66 (1.39, |
| | NDUFA8 ZBTB26 ZBTB34 RALGPS1 | | | | | | | | 1.97) |
| | ZNF79 C9orf117 TRUB2 SPTAN1 ZER1 | | | | | | | | |
| | TBC1D13 ENDOG LRRC8A PHYHD1 | | | | | | | | |
| | NUP188 SH3GLB2 TOR1A C9orf78 | | | | | | | | |
| | USP20 GPR107 ABL1 PRRC2B TTF1 | | | | | | | | |
| | AK8 C9orf9 TSC1 ADAMTSL2 | | | | | | | | |
| | NCRNA00094 BRD3 KIAA0649 | | | | | | | | |
| | C9orf116 | | | | | | | | |
| 17 | LMAN1L CPLX3 COX5A FBXO22 ISL2 | 15 | q | 75,105,193 | 82,338,484 | 12 | 1 | 5.52E−09 | 1.65 (1.40, |
| | LINGO1 CRABP1 MORF4L1 RASGRF1 | | | | | | | | 1.96) |
| | TMED3 KIAA1024 MEX3B | | | | | | | | |
| 18 | PDE11A RBM45 WDR75 SLC40A1 | 2 | q | 178,487,976 | 196,933,536 | 7 | −1 | 2.20E−08 | 1.65 (1.38, |
| | ANKAR OSGEPL1 DNAH7 | | | | | | | | 1.97) |
| 19 | ARMCX4 ARMCX1 NXF5 ZMAT1 | 23 | q | 100,673,250 | 109,421,016 | 8 | −1 | 2.67E−08 | 1.65 (1.38, |
| | MCART6 MUM1L1 COL4A5 TMEM164 | | | | | | | | 1.97) |
| 20 | PNKP FAM71E1 ZNF175 FLJ30403 | 19 | q | 50,364,459 | 55,099,027 | 18 | −1 | 4.07E−08 | 1.64 (1.37, |
| | ZNF616 ZNF836 ZNF610 ZNF528 | | | | | | | | 1.96) |
| | ZNF808 ZNF701 ZNF137P ZNF415 | | | | | | | | |
| | ZNF347 ZNF665 ZNF761 ZNF813 | | | | | | | | |
| | PRPF31 LILRA2 | | | | | | | | |
| 21 | LRP11 RAET1K ULBP3 PPP1R14C IYD | 6 | q | 150,139,893 | 159,240,456 | 6 | 1 | 5.68E−08 | 1.64 (1.37, |
| | EZR | | | | | | | | 1.96) |
| 22 | TMSB10 KCMF1 SH2D6 TMEM150A | 2 | p | 85,132,762 | 86,369,280 | 6 | 1 | 1.28E−08 | 1.63 (1.38, |
| | USP39 PTCD3 | | | | | | | | 1.93) |
| 23 | PYY NAGS TMEM101 TMUB2 | 17 | q | 42,030,106 | 45,915,079 | 12 | −1 | 4.37E−08 | 1.63 (1.37, |
| | CCDC103 PLCD3 ACBD4 MAP3K14 | | | | | | | | 1.95) |
| | LOC100128977 MAPT ARL17A LRRC46 | | | | | | | | |
| 24 | VNN3 LOC285735 MAP7 PERP HEBP2 | 6 | q | 133,043,925 | 144,152,322 | 9 | 1 | 3.64E−09 | 1.61 (1.38, |
| | NHSL1 GPR126 HIVEP2 PHACTR2 | | | | | | | | 1.89) |
| 25 | FLJ31306 TOMM20L JKAMP C14orf38 | 14 | q | 58,732,082 | 60,601,532 | 6 | −1 | 1.28E−07 | 1.61 (1.35, |
| | RTN1 C14orf135 | | | | | | | | 191) |
| 26 | ZBTB4 CYB5D1 LOC284023 VAMP2 | 17 | p | 7,362,684 | 19,771,239 | 17 | −1 | 1.06E−07 | 1.59 (1.34, |
| | C17orf68 SLC25A35 ARHGEF15 | | | | | | | | 1.89) |
| | WDR16 MYH8 C17orf48 TEKT3 PIGL | | | | | | | | |
| | CENPV ZNF287 LRRC48 SLC5A10 ULK2 | | | | | | | | |
| 27 | STX17 FSD1L FKTN PTGR1 C9orf80 | 9 | q | 102,668,914 | 116,191,879 | 6 | −1 | 2.21E−07 | 1.59 (1.34, |
| | C9orf43 | | | | | | | | 1.90) |
| 28 | SLC7A8 HOMEZ BCL2L2 ZFHX2 DHRS4 | 14 | q | 23,594,503 | 24,809,242 | 8 | −1 | 3.31E−07 | 1.59 (1.33, |
| | FITM1 GMPR2 RIPK3 | | | | | | | | 1.90) |
| 29 | SFXN1 CPLX2 FAF2 CDHR2 GPRIN1 | 5 | q | 174,905,513 | 176,727,214 | 8 | 1 | 8.24E−10 | 1.59 (1.37, |
| | TSPAN17 UIMC1 NSD1 | | | | | | | | 1.84) |
| 30 | ITLN2 PVRL4 NUF2 MPZL1 TBX19 F5 | 1 | q | 160,914,815 | 174,964,445 | 9 | 1 | 2.73E−07 | 1.59 (1.33, |
| | C1orf9 PRDX6 RABGAP1L | | | | | | | | 1.89) |
| 31 | C19orf39 EPOR ZNF627 ZNF441 | 19 | p | 11,485,382 | 12,792,701 | 21 | −1 | 2.31E−07 | 1.58 (1.33, |
| | ZNF491 ZNF439 ZNF763 ZNF433 | | | | | | | | 1.88) |
| | ZNF844 ZNF788 ZNF20 ZNF625 | | | | | | | | |
| | ZNF136 ZNF44 ZNF563 ZNF442 | | | | | | | | |
| | ZNF799 ZNF709 ZNF564 ZNF490 DHPS | | | | | | | | |
| 32 | C1orf88 DDX20 PHTF1 RSBN1 SYT6 | 1 | p | 111,889,194 | 119,683,295 | 9 | −1 | 3.10E−07 | 1.57 (1.32, |
| | ATP1A1OS TRIM45 SPAG17 WARS2 | | | | | | | | 1.87) |
| 33 | METTL7B SARNP TMEM194A NXPH4 | 12 | q | 56,075,329 | 59,314,319 | 6 | 1 | 2.92E−07 | 1.57 (1.32, |
| | SHMT2 LRIG3 | | | | | | | | 1.86) |
| 34 | SCFD2 TMEM165 SRP72 UBA6 | 4 | q | 53,739,150 | 83,812,335 | 21 | 1 | 2.80E−12 | 1.56 (1.38, |
| | TMPRSS11E UGT2B10 UGT2B7 | | | | | | | | 1.77) |
| | UGT2A1 AMBN GRSF1 ANKRD17 | | | | | | | | |
| | EPGN RCHY1 G3BP2 USO1 SDAD1 | | | | | | | | |
| | NUP54 SCARB2 CXCL13 RASGEF1B | | | | | | | | |
| | SEC31A | | | | | | | | |
| 35 | KIAA1310 UNC50 TSGA10 LIPT1 AFF3 | 2 | q | 97,258,891 | 110,962,639 | 6 | −1 | 5.42E−07 | 1.56 (1.31, |
| | NPHP1 | | | | | | | | 1.86) |
| 36 | FAM35A ATAD1 FLJ37201 RPP30 | 10 | q | 88,854,952 | 93,392,858 | 6 | −1 | 5.25E−07 | 1.55 (1.31, |
| | LOC100188947 PPP1R3C | | | | | | | | 1.84) |
| 37 | FANCI LOC254559 RHCG C15orf42 | 15 | q | 89,787,193 | 91,537,881 | 11 | 1 | 2.52E−10 | 1.55 (1.35, |
| | ZNF710 IDH2 SEMA4B CIB1 NGRN | | | | | | | | 1.77) |
| | BLM PRC1 | | | | | | | | |
| 38 | ARPP21 VILL DLEC1 CX3CR1 ZNF619 | 3 | p | 35,680,665 | 42,267,382 | 7 | −1 | 1.80E−06 | 1.54 (1.29, |
| | ULK4 TRAK1 | | | | | | | | 1.84) |
| 39 | TGFBR3 C1orf146 BCAR3 | 1 | p | 92,145,899 | 110,283,660 | 12 | −1 | 2.32E−06 | 1.54 (1.29, |
| | LOC100129046 ALG14 CCDC76 | | | | | | | | 1.83) |
| | AMY2B KIAA1324 MYBPHL GSTM4 | | | | | | | | |
| | GSTM2 GSTM3 | | | | | | | | |
| 40 | HEATR2 SUN1 INTS1 PSMG3 MAD1L1 | 7 | p | 766,337 | 6,523,849 | 7 | 1 | 1.11E−07 | 1.53 (1.31, |
| | EIF3B KDELR2 | | | | | | | | 1.79) |
| 41 | MSRA FAM66A FGF20 PCM1 SORBS3 | 8 | p | 9,911,829 | 24,814,383 | 6 | −1 | 2.62E−06 | 1.53 (1.28, |
| | NEFL | | | | | | | | 1.83) |
| 42 | ARPC5 IVNS1ABP FAM5C UCHL5 | 1 | q | 183,595,327 | 247,171,395 | 35 | 1 | 3.55E−06 | 1.52 (1.27, |
| | CDC73 CFHR5 ASPM KIF14 C1orf106 | | | | | | | | 1.82) |
| | LAD1 CHI3L1 NEK2 CENPF EPRS IARS2 | | | | | | | | |
| | RAB3GAP2 AIDA WDR26 SRP9 ACBD3 | | | | | | | | |
| | LIN9 GUK1 C1orf198 EGLN1 SIPA1L2 | | | | | | | | |
| | IRF2BP2 RBM34 B3GALNT2 CHRM3 | | | | | | | | |
| | KMO OPN3 EXO1 PPPDE1 AHCTF1 | | | | | | | | |
| | ZNF695 | | | | | | | | |
| 43 | POLQ PDIA5 PTPLB ALG1L PODXL2 | 3 | q | 121,150,272 | 127,790,526 | 6 | 1 | 3.84E−06 | 1.52 (1.27, |
| | SEC61A1 | | | | | | | | 1.82) |
| 44 | RAPGEF5 MGC87042 TOMM7 C7orf30 | 7 | p | 22,157,907 | 26,253,227 | 10 | 1 | 6.41E−11 | 1.51 (1.33, |
| | NPY MPP6 DFNA5 CYCS HNRNPA2B1 | | | | | | | | 1.70) |
| | CBX3 | | | | | | | | |
| 45 | CNGB3 RIPK2 LRRC69 RAD54B ESRP1 | 8 | q | 87,586,162 | 146,017,805 | 41 | 1 | 5.34E−06 | 1.51 (1.26, |
| | UQCRB PTDSS1 STK3 RNF19A PABPC1 | | | | | | | | 1.80) |
| | YWHAZ FLJ42969 GRHL2 NCALD | | | | | | | | |
| | SLC25A32 LRP12 EIF3E KCNV1 EXT1 | | | | | | | | |
| | TAF2 MTBP WDR67 FAM83A SQLE | | | | | | | | |
| | NDRG1 PTK2 SLC45A4 LYPD2 LY6D | | | | | | | | |
| | C8orf73 PYCRL TSTA3 LOC100128338 | | | | | | | | |
| | GRINA SPATC1 OPLAH GPAA1 | | | | | | | | |
| | HEATR7A ADCK5 CYHR1 RPL8 | | | | | | | | |
| 46 | CIC ZNF576 CADM4 ZNF221 ZNF230 | 19 | q | 42,788,816 | 44,905,777 | 11 | −1 | 5.14E−06 | 1.51 (1.26, |
| | ZNF224 LOC100379224 ZNF226 | | | | | | | | 1.79) |
| | ZNF227 ZFP112 ZNF285 | | | | | | | | |
| 47 | LDLRAD3 COMMD9 C11orf74 LRRC4C | 11 | p | 35,965,611 | 47,736,928 | 6 | −1 | 7.04E−06 | 1.50 (1.26, |
| | ALKBH3 AGBL2 | | | | | | | | 1.79) |
| 48 | SLC10A7 FBXW7 MND1 FGG ACCN5 | 4 | q | 147,175,136 | 156,841,558 | 6 | 1 | 4.49E−06 | 1.50 (1.26, |
| | TDO2 | | | | | | | | 1.78) |
| 49 | ZNF721 PIGG PDE6B MFSD7 PCGF3 | 4 | p | 433,778 | 8,442,452 | 14 | −1 | 6.53E−06 | 1.50 (1.26, |
| | CPLX1 TNIP2 C4orf44 RGS12 STK32B | | | | | | | | 1.78) |
| | CRMP1 WFS1 LOC100129931 ACOX3 | | | | | | | | |
| 50 | FLJ10661 LRTOMT ME3 KDM4D PGR | 11 | q | 71,498,557 | 117,156,404 | 16 | −1 | 4.48E−06 | 1.50 (1.26, |
| | TRPC6 C11orf70 ACAT1 FDXACB1 | | | | | | | | 1.78) |
| | DIXDC1 PIH1D2 C11orf34 TTC12 | | | | | | | | |
| | ANKK1 USP28 RNF214 | | | | | | | | |
| 51 | UBXN10 PINK1 NBPF3 HSPC157 | 1 | p | 20,512,577 | 38,100,595 | 22 | −1 | 9.94E−06 | 1.49 (1.25, |
| | ZBTB40 TCEA3 HMGCL MYOM3 | | | | | | | | 1.78) |
| | PAQR7 UBXN11 WDTC1 SRSF4 BAI2 | | | | | | | | |
| | CCDC28B IQCC MTMR9LP ADC TEKT2 | | | | | | | | |
| | MAP7D1 OSCP1 DNALI1 RSPO1 | | | | | | | | |
| 52 | TEX2 POLG2 DDX5 CCDC45 SMURF2 | 17 | q | 62,224,794 | 66,042,970 | 10 | 1 | 6.36E−09 | 1.49 (1.30, |
| | LOC146880 PLEKHM1P NOL11 BPTF | | | | | | | | 1.70) |
| | KPNA2 | | | | | | | | |
| 53 | LOC145837 LRRC49 CT62 CELF6 HEXA | 15 | q | 69,854,058 | 74,244,478 | 6 | −1 | 1.13E−05 | 1.49 (1.25, |
| | LOXL1 | | | | | | | | 1.78) |
| 54 | PPP1CB GALNT14 MEMO1 BIRC6 | 2 | p | 28,974,613 | 54,197,977 | 13 | 1 | 8.57E−06 | 1.49 (1.25, |
| | FAM98A ATL2 EML4 LRPPRC PPM1B | | | | | | | | 1.77) |
| | C2orf34 SRBD1 EPCAM PSME4 | | | | | | | | |
| 55 | ZBTB1 C14orf50 TMEM229B EXD2 | 14 | q | 64,971,291 | 92,572,965 | 17 | −1 | 1.09E−05 | 1.48 (1.24, |
| | GALNTL1 UPF0639 SRSF5 DNAL1 | | | | | | | | 1.77) |
| | C14orf45 EIF2B2 KIAA1737 POMT2 | | | | | | | | |
| | STON2 GALC TTC8 KCNK13 ATXN3 | | | | | | | | |
| 56 | NSUN2 SRD5A1 PAPD7 MTRR CCT5 | 5 | p | 6,599,351 | 33,468,196 | 12 | 1 | 1.31E−05 | 1.48 (1.24, |
| | MARCH6 DAP DROSHA C5orf22 | | | | | | | | 1.77) |
| | GOLPH3 ZFR TARS | | | | | | | | |
| 57 | PRR11 CLTC PTRH2 TMEM49 HEATR6 | 17 | q | 57,232,859 | 61,941,739 | 17 | 1 | 4.17E−08 | 1.48 (1.29, |
| | C17orf64 BCAS3 TBX4 MED13 DCAF7 | | | | | | | | 1.70) |
| | STRADA CCDC47 DDX42 FTSJ3 PSMC5 | | | | | | | | |
| | SMARCD2 TCAM1P | | | | | | | | |
| 58 | KIAA1609 USP10 KIAA0182 COX4I1 | 16 | q | 84,509,965 | 90,085,937 | 15 | 1 | 1.86E−05 | 1.48 (1.23, |
| | JPH3 SLC7A5 BANP ZC3H18 MVD | | | | | | | | 1.76) |
| | ANKRD11 CHMP1A LOC100128881 | | | | | | | | |
| | FANCA DEF8 DBNDD1 | | | | | | | | |
| 59 | PHIP TTK ME1 RIPPLY2 ATG5 PDSS2 | 6 | q | 79,644,135 | 107,780,779 | 6 | 1 | 1.11E−05 | 1.47 (1.24, |
| | | | | | | | | | 1.75) |
| 60 | KIF4A RLIM MAGT1 COX7B PGK1 | 23 | q | 69,509,878 | 91,878,228 | 7 | 1 | 1.88E−05 | 1.47 (1.23, |
| | TAF9B PCDH11X | | | | | | | | 1.75) |
| 61 | FLYWCH2 MMP25 MGC3771 ZNF263 | 16 | p | 2,933,195 | 31,519,706 | 47 | −1 | 1.24E−05 | 1.47 (1.23, |
| | TIGD7 ZNF434 ZNF174 ZNF597 NAT15 | | | | | | | | 1.74) |
| | CLUAP1 CREBBP GLIS2 C16orf5 | | | | | | | | |
| | MGRN1 C16orf71 NAGPA ABAT | | | | | | | | |
| | NUBP1 CLEC16A MKL2 NOMO2 | | | | | | | | |
| | ACSM5 LYRM1 C16orf52 GGA2 RBBP6 | | | | | | | | |
| | AQP8 NSMCE1 FLJ21408 KIAA0556 | | | | | | | | |
| | CCDC101 MVP YPEL3 LOC595101 | | | | | | | | |
| | TBC1D10B SEPT1 ZNF747 PHKG2 | | | | | | | | |
| | BCL7C HSD3B7 STX1B ZNF668 ZNF646 | | | | | | | | |
| | ZNF843 ARMC5 TGFB1I1 C16orf58 | | | | | | | | |
| 62 | NUP93 HERPUD1 COQ9 GPR56 | 16 | q | 56,764,016 | 70,719,954 | 22 | 1 | 2.53E−05 | 1.46 (1.23, |
| | MMP15 CNOT1 CDH8 DYNC1LI2 CBFB | | | | | | | | 1.75) |
| | GFOD2 RANBP10 NUTF2 DUS2L ESRP2 | | | | | | | | |
| | CYB5B WWP2 PDXDC2P AARS | | | | | | | | |
| | DDX19A COG4 SF3B3 MTSS1L | | | | | | | | |
| 63 | ZNF554 PIAS4 SIRT6 ANKRD24 | 19 | p | 2,819,871 | 6,751,529 | 12 | −1 | 3.03E−05 | 1.46 (1.22, |
| | CCDC94 HDGFRP2 KDM4B DUS3L | | | | | | | | 1.75) |
| | RANBP3 ACSBG2 MLLT1 TRIP10 | | | | | | | | |
| 64 | TMEM169 CTDSP1 PLCD4 RNF25 | 2 | q | 216,946,588 | 220,101,391 | 6 | −1 | 2.88E−05 | 1.46 (1.22, |
| | STK36 ANKZF1 | | | | | | | | 1.74) |
| 65 | C17orf97 RNMTL1 ABR YWHAE | 17 | p | 260,117 | 7,293,092 | 34 | −1 | 2.02E−05 | 1.45 (1.22, |
| | MYO1C INPP5K SCARF1 RTN4RL1 | | | | | | | | 1.73) |
| | SMG6 SRR SGSM2 METT10D TRPV1 | | | | | | | | |
| | SHPK C17orf85 ZZEF1 CYBSD2 PLD2 | | | | | | | | |
| | MINK1 CHRNE ENO3 CAMTA2 INCA1 | | | | | | | | |
| | ZFP3 ZNF594 LOC100130950 RABEP1 | | | | | | | | |
| | RPAIN KIAA0753 ALOX12 C17orf49 | | | | | | | | |
| | PHF23 GPS2 TNK1 | | | | | | | | |
| 66 | SHCBP1 ORC6 ITFG1 PHKB HEATR3 | 16 | q | 46,614,467 | 56,485,261 | 7 | 1 | 3.60E−05 | 1.45 (1.22, |
| | BRD7 NUDT21 | | | | | | | | 1.73) |
| 67 | C9orf68 C9orf46 ACER2 IFT74 | 9 | p | 4,598,315 | 34,620,496 | 7 | −1 | 3.62E−05 | 1.45 (1.22, |
| | NDUFB6 SMU1 DCTN3 | | | | | | | | 1.73) |
| 68 | BOD1L FAM184B DCAF16 PACRGL | 4 | p | 13,570,365 | 26,756,918 | 7 | −1 | 4.2E−05 | 1.45 (1.21, |
| | SEPSECS ANAPC4 TBC1D19 | | | | | | | | 1.73) |
| 69 | ATP11B MCCC1 YEATS2 ALG3 IGF2BP2 | 3 | q | 182,511,290 | 186,524,484 | 6 | 1 | 4.14E−05 | 1.45 (1.21, |
| | RFC4 | | | | | | | | 1.73) |
| 70 | PIK3C3 SETBP1 SLC14A2 HAUS1 | 18 | q | 39,535,198 | 47,814,692 | 7 | −1 | 4.57E−05 | 1.44 (1.21, |
| | HDHD2 CCDC11 CXXC1 | | | | | | | | 1.72) |
| 71 | FANCD2 C3orf24 MRPS25 COL6A4P1 | 3 | p | 10,068,112 | 23,632,296 | 6 | 1 | 9.8E−05 | 1.42 (1.19, |
| | SGOL1 UBE2E2 | | | | | | | | 1.70) |
| 72 | C14orf132 AK7 EVL DEGS2 ZNF839 | 14 | q | 96,505,660 | 105,461,855 | 6 | −1 | 0.000135 | 1.41 (1.18, |
| | C14orf79 | | | | | | | | 1.69) |
| 73 | C15orf23 CASC5 RAD51 RHOV INO80 | 15 | q | 40,674,921 | 52,861,213 | 24 | 1 | 8.24E−07 | 1.40 (1.23, |
| | OIP5 NUSAP1 ELL3 SPATA5L1 SLC12A1 | | | | | | | | 1.61) |
| | CEP152 SECISBP2L GALK2 GABPB1 | | | | | | | | |
| | USP8 TRPM7 SPPL2A AP4E1 | | | | | | | | |
| | TNFAIP8L3 CYP19A1 TMOD2 TMOD3 | | | | | | | | |
| | MAPK6 ARPP19 | | | | | | | | |
| 74 | GTSE1 BRD1 ZBED4 CRELD2 PIM3 | 22 | q | 46,692,637 | 50,656,045 | 7 | 1 | 1.81E−08 | 1.39 (1.24, |
| | TRABD SELO | | | | | | | | 1.56) |
| 75 | EVX1 HIBADH TAX1BP1 PLEKHA8 | 7 | p | 27,282,163 | 56,174,187 | 14 | 1 | 4.28E−05 | 1.37 (1.18, |
| | ZNRF2 NOD1 GGCT GARS ANLN VPS41 | | | | | | | | 1.60) |
| | CDK13 PPIA TBRG4 CHCHD2 | | | | | | | | |

TABLE 6B
Accession No. in Strings based on whole
gene analysis of the Providence Dataset

| String | | Accession | Chr. | |
|---|---|---|---|---|
| No. | Gene | No. | No. | Arm |
| 1 | SLCO6A1 | NM_173488 | chr5 | q |
| 1 | EFNA5 | NM_001962 | chr5 | q |
| 1 | ALDH7A1 | NM_001182 | chr5 | q |
| 1 | PRRC1 | NM_130809 | chr5 | q |
| 1 | VDAC1 | NM_003374 | chr5 | q |
| 1 | VDAC1 | NR_036625 | chr5 | q |
| 1 | VDAC1 | NR_036624 | chr5 | q |
| 1 | SEC24A | NM_021982 | chr5 | q |
| 1 | TRPC7 | NM_001167577 | chr5 | q |
| 1 | TRPC7 | NM_020389 | chr5 | q |
| 1 | TRPC7 | NM_001167576 | chr5 | q |
| 1 | KIF20A | NM_005733 | chr5 | q |
| 1 | CDC25C | NM_022809 | chr5 | q |
| 1 | CDC25C | NM_001790 | chr5 | q |
| 1 | HSPA9 | NM_004134 | chr5 | q |
| 1 | NRG2 | NM_013983 | chr5 | q |
| 1 | NRG2 | NM_013981 | chr5 | q |
| 1 | NRG2 | NM_001184935 | chr5 | q |
| 1 | NRG2 | NM_013982 | chr5 | q |
| 1 | NRG2 | NM_004883 | chr5 | q |
| 1 | PCDHGA6 | NM_018919 | chr5 | q |
| 1 | PCDHGB4 | NM_003736 | chr5 | q |
| 1 | DIAPH1 | NM_005219 | chr5 | q |
| 1 | DIAPH1 | NM_001079812 | chr5 | q |
| 2 | CYP4F8 | NM_007253 | chr19 | p |
| 2 | UCA1 | NR_015379 | chr19 | p |
| 2 | EPS15L1 | NM_021235 | chr19 | p |
| 2 | SIN3B | NM_015260 | chr19 | p |
| 2 | TMEM221 | NM_001190844 | chr19 | p |
| 2 | LOC284440 | NR_026956 | chr19 | p |
| 2 | ZNF682 | NM_001077349 | chr19 | p |
| 2 | ZNF682 | NM_033196 | chr19 | p |
| 2 | ZNF626 | NM_145297 | chr19 | p |
| 2 | ZNF626 | NM_001076675 | chr19 | p |
| 2 | ZNF85 | NM_003429 | chr19 | p |
| 2 | ZNF85 | NR_034060 | chr19 | p |
| 2 | ZNF708 | NM_021269 | chr19 | p |
| 2 | ZNF738 | NR_027130 | chr19 | p |
| 2 | ZNF429 | NM_001001415 | chr19 | p |
| 3 | DIO1 | NM_213593 | chr1 | p |
| 3 | DIO1 | NM_001039716 | chr1 | p |
| 3 | DIO1 | NM_001039715 | chr1 | p |
| 3 | DIO1 | NM_000792 | chr1 | p |
| 3 | HSPB11 | NM_016126 | chr1 | p |
| 3 | LRRC42 | NM_052940 | chr1 | p |
| 3 | FAM151A | NM_176782 | chr1 | p |
| 3 | C1orf168 | NM_001004303 | chr1 | p |
| 3 | DAB1 | NM_021080 | chr1 | p |
| 3 | OMA1 | NM_145243 | chr1 | p |
| 3 | WDR78 | NM_024763 | chr1 | p |
| 3 | WDR78 | NM_207014 | chr1 | p |
| 3 | SLC35D1 | NM_015139 | chr1 | p |
| 3 | HHLA3 | NM_001031693 | chr1 | p |
| 3 | HHLA3 | NM_001036645 | chr1 | p |
| 3 | HHLA3 | NM_001036646 | chr1 | p |
| 3 | HHLA3 | NR_027404 | chr1 | p |
| 3 | PTGER3 | NM_001126044 | chr1 | p |
| 3 | PTGER3 | NM_198716 | chr1 | p |
| 3 | PTGER3 | NM_198718 | chr1 | p |
| 3 | PTGER3 | NM_198715 | chr1 | p |
| 3 | PTGER3 | NM_198719 | chr1 | p |
| 3 | PTGER3 | NM_198714 | chr1 | p |
| 3 | PTGER3 | NR_028292 | chr1 | p |
| 3 | PTGER3 | NR_028293 | chr1 | p |
| 3 | PTGER3 | NM_198717 | chr1 | p |
| 3 | PTGER3 | NR_028294 | chr1 | p |
| 3 | NEGR1 | NM_173808 | chr1 | p |
| 3 | C1orf173 | NM_001002912 | chr1 | p |
| 3 | CRYZ | NM_001130042 | chr1 | p |
| 3 | CRYZ | NM_001130043 | chr1 | p |
| 3 | CRYZ | NM_001889 | chr1 | p |
| 3 | CRYZ | NM_001134759 | chr1 | p |
| 3 | TYW3 | NM_001162916 | chr1 | p |
| 3 | TYW3 | NM_138467 | chr1 | p |
| 3 | TYW3 | NR_027962 | chr1 | p |
| 3 | FAM73A | NM_198549 | chr1 | p |
| 3 | RPF1 | NM_025065 | chr1 | p |
| 4 | MICAL3 | NM_001122731 | chr22 | q |
| 4 | MICAL3 | NM_001136004 | chr22 | q |
| 4 | MICAL3 | NM_015241 | chr22 | q |
| 4 | GGT3P | NR_003267 | chr22 | q |
| 4 | DGCR6 | NM_005675 | chr22 | q |
| 4 | UFD1L | NM_001035247 | chr22 | q |
| 4 | UFD1L | NM_005659 | chr22 | q |
| 4 | CDC45 | NM_001178011 | chr22 | q |
| 4 | CDC45 | NM_001178010 | chr22 | q |
| 4 | CDC45 | NM_003504 | chr22 | q |
| 4 | PRAME | NM_206954 | chr22 | q |
| 4 | PRAME | NM_206953 | chr22 | q |
| 4 | PRAME | NM_006115 | chr22 | q |
| 4 | PRAME | NM_206956 | chr22 | q |
| 4 | PRAME | NM_206955 | chr22 | q |
| 4 | CHCHD10 | NM_213720 | chr22 | q |
| 4 | GSTTP1 | NR_003081 | chr22 | q |
| 4 | GGT1 | NM_005265 | chr22 | q |
| 4 | GGT1 | NM_013430 | chr22 | q |
| 4 | GGT1 | NM_001032365 | chr22 | q |
| 4 | GGT1 | NM_001032364 | chr22 | q |
| 4 | POM121L10P | NR_024593 | chr22 | q |
| 5 | ZNF37BP | NR_026777 | chr10 | q |
| 5 | ZNF487P | NR_026693 | chr10 | q |
| 5 | ZNF239 | NM_001099282 | chr10 | q |
| 5 | ZNF239 | NM_005674 | chr10 | q |
| 5 | ZNF239 | NM_001099284 | chr10 | q |
| 5 | ZNF239 | NM_001099283 | chr10 | q |
| 5 | LOC283033 | NR_033846 | chr10 | q |
| 5 | ANUBL1 | NM_001128324 | chr10 | q |
| 5 | ANUBL1 | NM_174890 | chr10 | q |
| 5 | SYT15 | NM_031912 | chr10 | q |
| 5 | SYT15 | NM_181519 | chr10 | q |
| 6 | PMAIP1 | NM_021127 | chr18 | q |
| 6 | BCL2 | NM_000657 | chr18 | q |
| 6 | BCL2 | NM_000633 | chr18 | q |
| 6 | SERPINB8 | NM_002640 | chr18 | q |
| 6 | SERPINB8 | NM_001031848 | chr18 | q |
| 6 | SERPINB8 | NM_198833 | chr18 | q |
| 6 | FBXO15 | NM_152676 | chr18 | q |
| 6 | FBXO15 | NM_001142958 | chr18 | q |
| 6 | LOC400657 | NR_024484 | chr18 | q |
| 6 | ZNF516 | NM_014643 | chr18 | q |
| 6 | LOC284276 | NR_015417 | chr18 | q |
| 6 | LOC100130522 | NR_028340 | chr18 | q |
| 6 | LOC100130522 | NR_028339 | chr18 | q |
| 7 | ERRFI1 | NM_018948 | chr1 | p |
| 7 | ENO1 | NM_001428 | chr1 | p |
| 7 | KIF1B | NM_183416 | chr1 | p |
| 7 | KIF1B | NM_015074 | chr1 | p |
| 7 | PGD | NM_002631 | chr1 | p |
| 7 | SRM | NM_003132 | chr1 | p |
| 7 | NPPB | NM_002521 | chr1 | p |
| 7 | DHRS3 | NM_004753 | chr1 | p |
| 8 | HINFP | NM_198971 | chr11 | q |
| 8 | HINFP | NM_015517 | chr11 | q |
| 8 | CCDC153 | NM_001145018 | chr11 | q |
| 8 | GRIK4 | NM_014619 | chr11 | q |
| 8 | TBCEL | NM_152715 | chr11 | q |
| 8 | TBCEL | NM_001130047 | chr11 | q |
| 8 | TECTA | NM_005422 | chr11 | q |
| 8 | VWA5A | NM_014622 | chr11 | q |
| 8 | VWA5A | NM_001130142 | chr11 | q |
| 8 | VWA5A | NM_198315 | chr11 | q |
| 8 | SPA17 | NM_017425 | chr11 | q |
| 8 | SLC37A2 | NM_001145290 | chr11 | q |
| 8 | SLC37A2 | NM_198277 | chr11 | q |
| 8 | KCNJ5 | NM_000890 | chr11 | q |
| 8 | ADAMTS8 | NM_007037 | chr11 | q |
| 9 | C9orf64 | NM_032307 | chr9 | q |
| 9 | LOC389765 | NR_029410 | chr9 | q |
| 9 | NAA35 | NM_024635 | chr9 | q |
| 9 | CDK20 | NM_001170639 | chr9 | q |
| 9 | CDK20 | NM_178432 | chr9 | q |
| 9 | CDK20 | NM_001170640 | chr9 | q |
| 9 | CDK20 | NM_001039803 | chr9 | q |
| 9 | CDK20 | NM_012119 | chr9 | q |
| 9 | SPIN1 | NM_006717 | chr9 | q |
| 9 | NXNL2 | NM_001161625 | chr9 | q |
| 9 | NXNL2 | NM_145283 | chr9 | q |
| 9 | SECISBP2 | NM_024077 | chr9 | q |
| 9 | NOL8 | NM_017948 | chr9 | q |
| 9 | NOL8 | NR_024020 | chr9 | q |
| 9 | CENPP | NM_001012267 | chr9 | q |
| 9 | ECM2 | NM_001393 | chr9 | q |
| 9 | ECM2 | NM_001197295 | chr9 | q |
| 9 | ECM2 | NM_001197296 | chr9 | q |
| 9 | BICD2 | NM_001003800 | chr9 | q |
| 9 | BICD2 | NM_015250 | chr9 | q |
| 9 | ANKRD19 | NR_026868 | chr9 | q |
| 9 | ZNF484 | NM_001007101 | chr9 | q |
| 9 | ZNF484 | NM_031486 | chr9 | q |
| 9 | FGD3 | NM_001083536 | chr9 | q |
| 9 | FGD3 | NM_033086 | chr9 | q |
| 9 | SUSD3 | NM_145006 | chr9 | q |
| 9 | C9orf89 | NM_032310 | chr9 | q |
| 9 | NINJ1 | NM_004148 | chr9 | q |
| 9 | FAM120AOS | NM_198841 | chr9 | q |
| 9 | FAM120A | NM_014612 | chr9 | q |
| 9 | PHF2 | NM_005392 | chr9 | q |
| 9 | FBP1 | NM_000507 | chr9 | q |
| 9 | FBP1 | NM_001127628 | chr9 | q |
| 9 | C9orf130 | NR_023389 | chr9 | q |
| 9 | C9orf130 | NR_023390 | chr9 | q |
| 9 | C9orf102 | NM_001010895 | chr9 | q |
| 9 | SLC35D2 | NM_007001 | chr9 | q |
| 9 | ZNF510 | NM_014930 | chr9 | q |
| 9 | ZNF782 | NM_001001662 | chr9 | q |
| 9 | LOC57653 | NR_036527 | chr9 | q |
| 9 | LOC57653 | NR_036529 | chr9 | q |
| 9 | LOC57653 | NR_036528 | chr9 | q |
| 9 | TSTD2 | NM_139246 | chr9 | q |
| 9 | NCBP1 | NM_002486 | chr9 | q |
| 9 | XPA | NR_027302 | chr9 | q |
| 9 | XPA | NM_000380 | chr9 | q |
| 9 | C9orf156 | NM_016481 | chr9 | q |
| 10 | NLRP8 | NM_176811 | chr19 | q |
| 10 | LOC386758 | NR_037159 | chr19 | q |
| 10 | LOC386758 | NR_037161 | chr19 | q |
| 10 | LOC386758 | NR_037160 | chr19 | q |
| 10 | ZFP28 | NM_020828 | chr19 | q |
| 10 | ZNF71 | NM_021216 | chr19 | q |
| 10 | ZNF835 | NM_001005850 | chr19 | q |
| 10 | ZNF211 | NM_198855 | chr19 | q |
| 10 | ZNF211 | NM_006385 | chr19 | q |
| 10 | C19orf18 | NM_152474 | chr19 | q |
| 10 | ZNF132 | NM_003433 | chr19 | q |
| 11 | TCERG1 | NM_001040006 | chr5 | q |
| 11 | TCERG1 | NM_006706 | chr5 | q |
| 11 | SPINK5 | NM_006846 | chr5 | q |
| 11 | SPINK5 | NM_001127699 | chr5 | q |
| 11 | SPINK5 | NM_001127698 | chr5 | q |
| 11 | IRGM | NM_001145805 | chr5 | q |
| 11 | GLRA1 | NM_000171 | chr5 | q |
| 11 | GLRA1 | NM_001146040 | chr5 | q |
| 11 | LARP1 | NM_015315 | chr5 | q |
| 11 | CLINT1 | NM_014666 | chr5 | q |
| 11 | CLINT1 | NM_001195556 | chr5 | q |
| 11 | CLINT1 | NM_001195555 | chr5 | q |
| 11 | RNF145 | NM_001199383 | chr5 | q |
| 11 | RNF145 | NM_001199382 | chr5 | q |
| 11 | RNF145 | NM_144726 | chr5 | q |
| 11 | RNF145 | NM_001199381 | chr5 | q |
| 11 | RNF145 | NM_001199380 | chr5 | q |
| 11 | LOC285629 | NR_027111 | chr5 | q |
| 12 | NOSTRIN | NM_001171631 | chr2 | q |
| 12 | NOSTRIN | NM_052946 | chr2 | q |
| 12 | NOSTRIN | NM_001039724 | chr2 | q |
| 12 | NOSTRIN | NM_001171632 | chr2 | q |
| 12 | LRP2 | NM_004525 | chr2 | q |
| 12 | BBS5 | NM_152384 | chr2 | q |
| 12 | KBTBD10 | NM_006063 | chr2 | q |
| 12 | PPIG | NM_004792 | chr2 | q |
| 12 | C2orf77 | NM_001085447 | chr2 | q |
| 12 | PHOSPHO2 | NM_001008489 | chr2 | q |
| 12 | PHOSPHO2 | NM_001199287 | chr2 | q |
| 12 | PHOSPHO2 | NM_001199288 | chr2 | q |
| 12 | PHOSPHO2 | NM_001199285 | chr2 | q |
| 12 | PHOSPHO2 | NM_001199286 | chr2 | q |
| 12 | KLHL23 | NM_144711 | chr2 | q |
| 12 | KLHL23 | NM_001199290 | chr2 | q |
| 12 | DCAF17 | NM_001164821 | chr2 | q |
| 12 | DCAF17 | NR_028482 | chr2 | q |
| 12 | DCAF17 | NM_025000 | chr2 | q |
| 12 | CYBRD1 | NM_001127383 | chr2 | q |
| 12 | CYBRD1 | NM_024843 | chr2 | q |
| 12 | DYNC1I2 | NM_001378 | chr2 | q |
| 12 | SLC25A12 | NM_003705 | chr2 | q |
| 13 | RRP8 | NM_015324 | chr11 | p |
| 13 | DCHS1 | NM_003737 | chr11 | p |
| 13 | ZNF214 | NM_013249 | chr11 | p |
| 13 | SYT9 | NM_175733 | chr11 | p |
| 13 | TRIM66 | NM_014818 | chr11 | p |
| 13 | NRIP3 | NM_020645 | chr11 | p |
| 13 | SCUBE2 | NM_001170690 | chr11 | p |
| 13 | SCUBE2 | NM_020974 | chr11 | p |
| 14 | RAD52 | NM_134424 | chr12 | p |
| 14 | DCP1B | NM_152640 | chr12 | p |
| 14 | PARP11 | NM_020367 | chr12 | p |
| 14 | C12orf5 | NM_020375 | chr12 | p |
| 14 | VAMP1 | NM_014231 | chr12 | p |
| 14 | VAMP1 | NM_016830 | chr12 | p |
| 14 | VAMP1 | NM_199245 | chr12 | p |
| 14 | ACRBP | NM_032489 | chr12 | p |
| 14 | C12orf53 | NM_153685 | chr12 | p |
| 14 | LRRC23 | NM_006992 | chr12 | p |
| 14 | LRRC23 | NM_201650 | chr12 | p |
| 14 | LRRC23 | NM_001135217 | chr12 | p |
| 14 | ATN1 | NM_001007026 | chr12 | p |
| 14 | ATN1 | NM_001940 | chr12 | p |
| 14 | FOXJ2 | NM_018416 | chr12 | p |
| 14 | CLEC4A | NM_016184 | chr12 | p |
| 14 | CLEC4A | NM_194450 | chr12 | p |
| 14 | CLEC4A | NM_194448 | chr12 | p |
| 14 | CLEC4A | NM_194447 | chr12 | p |
| 14 | FAM66C | NR_026788 | chr12 | p |
| 14 | FAM90A1 | NM_018088 | chr12 | p |
| 14 | CREBL2 | NM_001310 | chr12 | p |
| 14 | DDX47 | NM_016355 | chr12 | p |
| 14 | DDX47 | NM_201224 | chr12 | p |
| 14 | HEBP1 | NM_015987 | chr12 | p |
| 14 | GSG1 | NM_031289 | chr12 | p |
| 14 | GSG1 | NM_153823 | chr12 | p |
| 14 | GSG1 | NM_001080555 | chr12 | p |
| 14 | GSG1 | NM_001080554 | chr12 | p |
| 14 | RERG | NM_032918 | chr12 | p |
| 14 | RERG | NM_001190726 | chr12 | p |
| 14 | DERA | NM_015954 | chr12 | p |
| 15 | FRMD8 | NM_031904 | chr11 | q |
| 15 | CFL1 | NM_005507 | chr11 | q |
| 15 | YIF1A | NM_020470 | chr11 | q |
| 15 | TMEM151A | NM_153266 | chr11 | q |
| 15 | NDUFV1 | NM_007103 | chr11 | q |
| 15 | NDUFV1 | NM_001166102 | chr11 | q |
| 15 | NUDT8 | NM_181843 | chr11 | q |
| 15 | TBX10 | NM_005995 | chr11 | q |
| 15 | CHKA | NM_001277 | chr11 | q |
| 15 | CHKA | NM_212469 | chr11 | q |
| 15 | C11orf24 | NM_022338 | chr11 | q |
| 15 | DHCR7 | NM_001360 | chr11 | q |
| 15 | DHCR7 | NM_001163817 | chr11 | q |
| 16 | ASTN2 | NM_001184735 | chr9 | q |
| 16 | ASTN2 | NM_198188 | chr9 | q |
| 16 | ASTN2 | NM_001184734 | chr9 | q |
| 16 | ASTN2 | NM_014010 | chr9 | q |
| 16 | ASTN2 | NM_198187 | chr9 | q |
| 16 | ASTN2 | NM_198186 | chr9 | q |
| 16 | TRIM32 | NM_001099679 | chr9 | q |
| 16 | TRIM32 | NM_012210 | chr9 | q |
| 16 | MEGF9 | NM_001080497 | chr9 | q |
| 16 | C5 | NM_001735 | chr9 | q |
| 16 | RAB14 | NM_016322 | chr9 | q |
| 16 | NDUFA8 | NM_014222 | chr9 | q |
| 16 | ZBTB26 | NM_020924 | chr9 | q |
| 16 | ZBTB34 | NM_001099270 | chr9 | q |
| 16 | RALGPS1 | NM_014636 | chr9 | q |
| 16 | RALGPS1 | NM_001190728 | chr9 | q |
| 16 | RALGPS1 | NM_001190729 | chr9 | q |
| 16 | RALGPS1 | NM_001190730 | chr9 | q |
| 16 | ZNF79 | NM_007135 | chr9 | q |
| 16 | C9orf117 | NM_001012502 | chr9 | q |
| 16 | TRUB2 | NM_015679 | chr9 | q |
| 16 | SPTAN1 | NM_003127 | chr9 | q |
| 16 | SPTAN1 | NM_001130438 | chr9 | q |
| 16 | SPTAN1 | NM_001195532 | chr9 | q |
| 16 | ZER1 | NM_006336 | chr9 | q |
| 16 | TBC1D13 | NM_018201 | chr9 | q |
| 16 | ENDOG | NM_004435 | chr9 | q |
| 16 | LRRC8A | NM_001127245 | chr9 | q |
| 16 | LRRC8A | NM_019594 | chr9 | q |
| 16 | LRRC8A | NM_001127244 | chr9 | q |
| 16 | PHYHD1 | NM_001100877 | chr9 | q |
| 16 | PHYHD1 | NM_001100876 | chr9 | q |
| 16 | PHYHD1 | NM_174933 | chr9 | q |
| 16 | NUP188 | NM_015354 | chr9 | q |
| 16 | SH3GLB2 | NM_020145 | chr9 | q |
| 16 | TOR1A | NM_000113 | chr9 | q |
| 16 | C9orf78 | NM_016520 | chr9 | q |
| 16 | USP20 | NM_001110303 | chr9 | q |
| 16 | USP20 | NM_006676 | chr9 | q |
| 16 | USP20 | NM_001008563 | chr9 | q |
| 16 | GPR107 | NM_020960 | chr9 | q |
| 16 | GPR107 | NM_001136557 | chr9 | q |
| 16 | GPR107 | NM_001136558 | chr9 | q |
| 16 | ABL1 | NM_007313 | chr9 | q |
| 16 | ABL1 | NM_005157 | chr9 | q |
| 16 | PRRC2B | NM_013318 | chr9 | q |
| 16 | TTF1 | NM_007344 | chr9 | q |
| 16 | AK8 | NM_152572 | chr9 | q |
| 16 | C9orf9 | NM_018956 | chr9 | q |
| 16 | TSC1 | NM_001162427 | chr9 | q |
| 16 | TSC1 | NM_000368 | chr9 | q |
| 16 | TSC1 | NM_001162426 | chr9 | q |
| 16 | ADAMTSL2 | NM_001145320 | chr9 | q |
| 16 | ADAMTSL2 | NM_014694 | chr9 | q |
| 16 | NCRNA00094 | NR_015427 | chr9 | q |
| 16 | BRD3 | NM_007371 | chr9 | q |
| 16 | KIAA0649 | NM_014811 | chr9 | q |
| 16 | C9orf116 | NM_144654 | chr9 | q |
| 16 | C9orf116 | NM_001048265 | chr9 | q |
| 17 | LMAN1L | NM_021819 | chr15 | q |
| 17 | CPLX3 | NM_001030005 | chr15 | q |
| 17 | COX5A | NM_004255 | chr15 | q |
| 17 | FBXO22 | NM_012170 | chr15 | q |
| 17 | FBXO22 | NR_037623 | chr15 | q |
| 17 | FBXO22 | NM_147188 | chr15 | q |
| 17 | ISL2 | NM_145805 | chr15 | q |
| 17 | LINGO1 | NM_032808 | chr15 | q |
| 17 | CRABP1 | NM_004378 | chr15 | q |
| 17 | MORF4L1 | NM_006791 | chr15 | q |
| 17 | MORF4L1 | NM_206839 | chr15 | q |
| 17 | RASGRF1 | NM_153815 | chr15 | q |
| 17 | RASGRF1 | NM_002891 | chr15 | q |
| 17 | RASGRF1 | NM_001145648 | chr15 | q |
| 17 | TMED3 | NM_007364 | chr15 | q |
| 17 | KIAA1024 | NM_015206 | chr15 | q |
| 17 | MEX3B | NM_032246 | chr15 | q |
| 18 | PDE11A | NM_001077197 | chr2 | q |
| 18 | PDE11A | NM_016953 | chr2 | q |
| 18 | PDE11A | NM_001077196 | chr2 | q |
| 18 | PDE11A | NM_001077358 | chr2 | q |
| 18 | RBM45 | NM_152945 | chr2 | q |
| 18 | WDR75 | NM_032168 | chr2 | q |
| 18 | SLC40A1 | NM_014585 | chr2 | q |
| 18 | ANKAR | NM_144708 | chr2 | q |
| 18 | OSGEPL1 | NM_022353 | chr2 | q |
| 18 | DNAH7 | NM_018897 | chr2 | q |
| 19 | ARMCX4 | NR_028407 | chrX | q |
| 19 | ARMCX1 | NM_016608 | chrX | q |
| 19 | NXF5 | NR_028089 | chrX | q |
| 19 | NXF5 | NM_032946 | chrX | q |
| 19 | ZMAT1 | NR_036431 | chrX | q |
| 19 | ZMAT1 | NM_001011657 | chrX | q |
| 19 | MCART6 | NM_001012755 | chrX | q |
| 19 | MUM1L1 | NM_152423 | chrX | q |
| 19 | MUM1L1 | NM_001171020 | chrX | q |
| 19 | COL4A5 | NM_033380 | chrX | q |
| 19 | COL4A5 | NM_000495 | chrX | q |
| 19 | TMEM164 | NM_017698 | chrX | q |
| 19 | TMEM164 | NM_032227 | chrX | q |
| 20 | PNKP | NM_007254 | chr19 | q |
| 20 | FAM71E1 | NM_138411 | chr19 | q |
| 20 | ZNF175 | NM_007147 | chr19 | q |
| 20 | FLJ30403 | NR_034159 | chr19 | q |
| 20 | ZNF616 | NM_178523 | chr19 | q |
| 20 | ZNF836 | NM_001102657 | chr19 | q |
| 20 | ZNF610 | NM_001161427 | chr19 | q |
| 20 | ZNF610 | NM_001161425 | chr19 | q |
| 20 | ZNF610 | NM_001161426 | chr19 | q |
| 20 | ZNF610 | NM_173530 | chr19 | q |
| 20 | ZNF528 | NM_032423 | chr19 | q |
| 20 | ZNF808 | NM_001039886 | chr19 | q |
| 20 | ZNF701 | NM_018260 | chr19 | q |
| 20 | ZNF701 | NM_001172655 | chr19 | q |
| 20 | ZNF137P | NR_023311 | chr19 | q |
| 20 | ZNF415 | NM_001136038 | chr19 | q |
| 20 | ZNF415 | NM_001164309 | chr19 | q |
| 20 | ZNF415 | NR_028343 | chr19 | q |
| 20 | ZNF415 | NM_018355 | chr19 | q |
| 20 | ZNF347 | NM_001172674 | chr19 | q |
| 20 | ZNF347 | NM_032584 | chr19 | q |
| 20 | ZNF347 | NM_001172675 | chr19 | q |
| 20 | ZNF665 | NM_024733 | chr19 | q |
| 20 | ZNF761 | NM_001008401 | chr19 | q |
| 20 | ZNF813 | NM_001004301 | chr19 | q |
| 20 | PRPF31 | NM_015629 | chr19 | q |
| 20 | LILRA2 | NM_001130917 | chr19 | q |
| 20 | LILRA2 | NM_006866 | chr19 | q |
| 21 | LRP11 | NM_032832 | chr6 | q |
| 21 | RAET1K | NR_024045 | chr6 | q |
| 21 | ULBP3 | NM_024518 | chr6 | q |
| 21 | PPP1R14C | NM_030949 | chr6 | q |
| 21 | IYD | NM_001164694 | chr6 | q |
| 21 | IYD | NM_001164695 | chr6 | q |
| 21 | IYD | NM_203395 | chr6 | q |
| 21 | EZR | NM_001111077 | chr6 | q |
| 21 | EZR | NM_003379 | chr6 | q |
| 22 | TMSB10 | NM_021103 | chr2 | p |
| 22 | KCMF1 | NM_020122 | chr2 | p |
| 22 | SH2D6 | NM_198482 | chr2 | p |
| 22 | TMEM150A | NR_033179 | chr2 | p |
| 22 | TMEM150A | NM_001031738 | chr2 | p |
| 22 | USP39 | NM_006590 | chr2 | p |
| 22 | PTCD3 | NM_017952 | chr2 | p |
| 23 | PYY | NM_004160 | chr17 | q |
| 23 | NAGS | NM_153006 | chr17 | q |
| 23 | TMEM101 | NM_032376 | chr17 | q |
| 23 | TMUB2 | NM_001076674 | chr17 | q |
| 23 | TMUB2 | NM_024107 | chr17 | q |
| 23 | TMUB2 | NM_177441 | chr17 | q |
| 23 | CCDC103 | NM_213607 | chr17 | q |
| 23 | PLCD3 | NM_133373 | chr17 | q |
| 23 | ACBD4 | NM_001135704 | chr17 | q |
| 23 | ACBD4 | NM_001135707 | chr17 | q |
| 23 | ACBD4 | NM_024722 | chr17 | q |
| 23 | ACBD4 | NM_001135706 | chr17 | q |
| 23 | ACBD4 | NM_001135705 | chr17 | q |
| 23 | MAP3K14 | NM_003954 | chr17 | q |
| 23 | LOC100128977 | NR_024559 | chr17 | q |
| 23 | MAPT | NM_001123066 | chr17 | q |
| 23 | MAPT | NM_001123067 | chr17 | q |
| 23 | MAPT | NM_016835 | chr17 | q |
| 23 | MAPT | NM_005910 | chr17 | q |
| 23 | MAPT | NM_016841 | chr17 | q |
| 23 | MAPT | NM_016834 | chr17 | q |
| 23 | ARL17A | NM_001113738 | chr17 | q |
| 23 | ARL17A | NM_001113738 | chr17 | q |
| 23 | ARL17A | NM_016632 | chr17 | q |
| 23 | ARL17A | NM_016632 | chr17 | q |
| 23 | LRRC46 | NM_033413 | chr17 | q |
| 24 | VNN3 | NR_028290 | chr6 | q |
| 24 | VNN3 | NR_028291 | chr6 | q |
| 24 | LOC285735 | NR_026969 | chr6 | q |
| 24 | MAP7 | NM_001198617 | chr6 | q |
| 24 | MAP7 | NM_001198616 | chr6 | q |
| 24 | MAP7 | NM_003980 | chr6 | q |
| 24 | MAP7 | NM_001198611 | chr6 | q |
| 24 | MAP7 | NM_001198615 | chr6 | q |
| 24 | MAP7 | NM_001198614 | chr6 | q |
| 24 | MAP7 | NM_001198618 | chr6 | q |
| 24 | MAP7 | NM_001198608 | chr6 | q |
| 24 | MAP7 | NM_001198609 | chr6 | q |
| 24 | MAP7 | NM_001198619 | chr6 | q |
| 24 | PERP | NM_022121 | chr6 | q |
| 24 | HEBP2 | NM_014320 | chr6 | q |
| 24 | NHSL1 | NM_020464 | chr6 | q |
| 24 | NHSL1 | NM_001144060 | chr6 | q |
| 24 | GPR126 | NM_198569 | chr6 | q |
| 24 | GPR126 | NM_020455 | chr6 | q |
| 24 | GPR126 | NM_001032395 | chr6 | q |
| 24 | GPR126 | NM_001032394 | chr6 | q |
| 24 | HIVEP2 | NM_006734 | chr6 | q |
| 24 | PHACTR2 | NM_001100166 | chr6 | q |
| 24 | PHACTR2 | NM_014721 | chr6 | q |
| 24 | PHACTR2 | NM_001100165 | chr6 | q |
| 24 | PHACTR2 | NM_001100164 | chr6 | q |
| 25 | FLJ31306 | NR_029435 | chr14 | q |
| 25 | FLJ31306 | NR_029434 | chr14 | q |
| 25 | TOMM20L | NM_207377 | chr14 | q |
| 25 | JKAMP | NM_001098625 | chr14 | q |
| 25 | JKAMP | NM_016475 | chr14 | q |
| 25 | C14orf38 | NM_001164399 | chr14 | q |
| 25 | RTN1 | NM_021136 | chr14 | q |
| 25 | RTN1 | NM_206852 | chr14 | q |
| 25 | RTN1 | NM_206857 | chr14 | q |
| 25 | C14orf135 | NM_022495 | chr14 | q |
| 26 | ZBTB4 | NM_001128833 | chr17 | p |
| 26 | ZBTB4 | NM_020899 | chr17 | p |
| 26 | CYB5D1 | NM_144607 | chr17 | p |
| 26 | LOC284023 | NR_024349 | chr17 | p |
| 26 | VAMP2 | NM_014232 | chr17 | p |
| 26 | C17orf68 | NM_025099 | chr17 | p |
| 26 | SLC25A35 | NM_201520 | chr17 | p |
| 26 | ARHGEF15 | NM_173728 | chr17 | p |
| 26 | ARHGEF15 | NM_025014 | chr17 | p |
| 26 | WDR16 | NM_145054 | chr17 | p |
| 26 | WDR16 | NM_001080556 | chr17 | p |
| 26 | MYH8 | NM_002472 | chr17 | p |
| 26 | C17orf48 | NM_020233 | chr17 | p |
| 26 | TEKT3 | NM_031898 | chr17 | p |
| 26 | PIGL | NM_004278 | chr17 | p |
| 26 | CENPV | NM_181716 | chr17 | p |
| 26 | ZNF287 | NM_020653 | chr17 | p |
| 26 | LRRC48 | NM_031294 | chr17 | p |
| 26 | LRRC48 | NM_001130092 | chr17 | p |
| 26 | LRRC48 | NM_001130091 | chr17 | p |
| 26 | LRRC48 | NM_001130090 | chr17 | p |
| 26 | SLC5A10 | NM_001042450 | chr17 | p |
| 26 | SLC5A10 | NM_152351 | chr17 | p |
| 26 | ULK2 | NM_014683 | chr17 | p |
| 26 | ULK2 | NM_001142610 | chr17 | p |
| 27 | STX17 | NM_017919 | chr9 | q |
| 27 | FSD1L | NM_001145313 | chr9 | q |
| 27 | FSD1L | NM_031919 | chr9 | q |
| 27 | FSD1L | NM_207647 | chr9 | q |
| 27 | FKTN | NM_001198963 | chr9 | q |
| 27 | FKTN | NM_006731 | chr9 | q |
| 27 | FKTN | NM_001079802 | chr9 | q |
| 27 | PTGR1 | NM_001146108 | chr9 | q |
| 27 | PTGR1 | NM_012212 | chr9 | q |
| 27 | PTGR1 | NM_001146109 | chr9 | q |
| 27 | C9orf80 | NM_021218 | chr9 | q |
| 27 | C9orf43 | NM_152786 | chr9 | q |
| 28 | SLC7A8 | NM_182728 | chr14 | q |
| 28 | SLC7A8 | NM_012244 | chr14 | q |
| 28 | HOMEZ | NM_020834 | chr14 | q |
| 28 | BCL2L2 | NM_004050 | chr14 | q |
| 28 | BCL2L2 | NM_001199839 | chr14 | q |
| 28 | ZFHX2 | NM_033400 | chr14 | q |
| 28 | DHRS4 | NM_021004 | chr14 | q |
| 28 | FITM1 | NM_203402 | chr14 | q |
| 28 | GMPR2 | NM_001002001 | chr14 | q |
| 28 | GMPR2 | NM_016576 | chr14 | q |
| 28 | GMPR2 | NM_001002000 | chr14 | q |
| 28 | GMPR2 | NM_001002002 | chr14 | q |
| 28 | RIPK3 | NM_006871 | chr14 | q |
| 29 | SFXN1 | NM_022754 | chr5 | q |
| 29 | CPLX2 | NM_006650 | chr5 | q |
| 29 | CPLX2 | NM_001008220 | chr5 | q |
| 29 | FAF2 | NM_014613 | chr5 | q |
| 29 | CDHR2 | NM_017675 | chr5 | q |
| 29 | CDHR2 | NM_001171976 | chr5 | q |
| 29 | GPRIN1 | NM_052899 | chr5 | q |
| 29 | TSPAN17 | NM_012171 | chr5 | q |
| 29 | TSPAN17 | NM_130465 | chr5 | q |
| 29 | TSPAN17 | NM_001006616 | chr5 | q |
| 29 | UIMC1 | NM_016290 | chr5 | q |
| 29 | UIMC1 | NM_001199298 | chr5 | q |
| 29 | UIMC1 | NM_001199297 | chr5 | q |
| 29 | NSD1 | NM_022455 | chr5 | q |
| 29 | NSD1 | NM_172349 | chr5 | q |
| 30 | ITLN2 | NM_080878 | chr1 | q |
| 30 | PVRL4 | NM_030916 | chr1 | q |
| 30 | NUF2 | NM_145697 | chr1 | q |
| 30 | NUF2 | NM_031423 | chr1 | q |
| 30 | MPZL1 | NM_003953 | chr1 | q |
| 30 | MPZL1 | NM_024569 | chr1 | q |
| 30 | MPZL1 | NM_001146191 | chr1 | q |
| 30 | TBX19 | NM_005149 | chr1 | q |
| 30 | F5 | NM_000130 | chr1 | q |
| 30 | C1orf9 | NM_016227 | chr1 | q |
| 30 | C1orf9 | NM_014283 | chr1 | q |
| 30 | PRDX6 | NM_004905 | chr1 | q |
| 30 | RABGAP1L | NM_014857 | chr1 | q |
| 30 | RABGAP1L | NM_001035230 | chr1 | q |
| 31 | C19orf39 | NM_175871 | chr19 | p |
| 31 | EPOR | NM_000121 | chr19 | p |
| 31 | EPOR | NR_033663 | chr19 | p |
| 31 | ZNF627 | NM_145295 | chr19 | p |
| 31 | ZNF441 | NM_152355 | chr19 | p |
| 31 | ZNF491 | NM_152356 | chr19 | p |
| 31 | ZNF439 | NM_152262 | chr19 | p |
| 31 | ZNF763 | NM_001012753 | chr19 | p |
| 31 | ZNF433 | NM_001080411 | chr19 | p |
| 31 | ZNF844 | NM_001136501 | chr19 | p |
| 31 | ZNF788 | NR_027049 | chr19 | p |
| 31 | ZNF20 | NM_021143 | chr19 | p |
| 31 | ZNF625 | NM_145233 | chr19 | p |
| 31 | ZNF136 | NM_003437 | chr19 | p |
| 31 | ZNF44 | NM_016264 | chr19 | p |
| 31 | ZNF44 | NM_001164276 | chr19 | p |
| 31 | ZNF563 | NM_145276 | chr19 | p |
| 31 | ZNF442 | NM_030824 | chr19 | p |
| 31 | ZNF799 | NM_001080821 | chr19 | p |
| 31 | ZNF709 | NM_152601 | chr19 | p |
| 31 | ZNF564 | NM_144976 | chr19 | p |
| 31 | ZNF490 | NM_020714 | chr19 | p |
| 31 | DHPS | NM_013407 | chr19 | p |
| 31 | DHPS | NM_001930 | chr19 | p |
| 31 | DHPS | NM_013406 | chr19 | p |
| 32 | C1orf88 | NM_181643 | chr1 | p |
| 32 | DDX20 | NM_007204 | chr1 | p |
| 32 | PHTF1 | NM_006608 | chr1 | p |
| 32 | RSBN1 | NM_018364 | chr1 | p |
| 32 | SYT6 | NM_205848 | chr1 | p |
| 32 | ATP1A1OS | NR_027646 | chr1 | p |
| 32 | ATP1A1OS | NR_027645 | chr1 | p |
| 32 | ATP1A1OS | NR_024124 | chr1 | p |
| 32 | ATP1A1OS | NR_024126 | chr1 | p |
| 32 | ATP1A1OS | NR_024125 | chr1 | p |
| 32 | TRIM45 | NM_001145635 | chr1 | p |
| 32 | TRIM45 | NM_025188 | chr1 | p |
| 32 | SPAG17 | NM_206996 | chr1 | p |
| 32 | WARS2 | NM_201263 | chr1 | p |
| 32 | WARS2 | NM_015836 | chr1 | p |
| 33 | METTL7B | NM_152637 | chr12 | q |
| 33 | SARNP | NR_026722 | chr12 | q |
| 33 | SARNP | NM_033082 | chr12 | q |
| 33 | SARNP | NR_026723 | chr12 | q |
| 33 | TMEM194A | NM_015257 | chr12 | q |
| 33 | TMEM194A | NM_001130963 | chr12 | q |
| 33 | NXPH4 | NM_007224 | chr12 | q |
| 33 | SHMT2 | NM_001166359 | chr12 | q |
| 33 | SHMT2 | NM_001166358 | chr12 | q |
| 33 | SHMT2 | NM_001166357 | chr12 | q |
| 33 | SHMT2 | NM_001166356 | chr12 | q |
| 33 | SHMT2 | NM_005412 | chr12 | q |
| 33 | SHMT2 | NR_029416 | chr12 | q |
| 33 | SHMT2 | NR_029417 | chr12 | q |
| 33 | SHMT2 | NR_029415 | chr12 | q |
| 33 | LRIG3 | NM_001136051 | chr12 | q |
| 33 | LRIG3 | NM_153377 | chr12 | q |
| 34 | SCFD2 | NM_152540 | chr4 | q |
| 34 | TMEM165 | NM_018475 | chr4 | q |
| 34 | SRP72 | NM_006947 | chr4 | q |
| 34 | UBA6 | NM_018227 | chr4 | q |
| 34 | TMPRSS11E | NM_014058 | chr4 | q |
| 34 | UGT2B10 | NM_001075 | chr4 | q |
| 34 | UGT2B10 | NM_001075 | chr4 | q |
| 34 | UGT2B10 | NM_001144767 | chr4 | q |
| 34 | UGT2B10 | NM_001144767 | chr4 | q |
| 34 | UGT2B7 | NM_001074 | chr4 | q |
| 34 | UGT2A1 | NM_006798 | chr4 | q |
| 34 | AMBN | NM_016519 | chr4 | q |
| 34 | GRSF1 | NM_002092 | chr4 | q |
| 34 | GRSF1 | NM_001098477 | chr4 | q |
| 34 | ANKRD17 | NM_198889 | chr4 | q |
| 34 | ANKRD17 | NM_032217 | chr4 | q |
| 34 | EPGN | NM_001013442 | chr4 | q |
| 34 | RCHY1 | NM_001009922 | chr4 | q |
| 34 | RCHY1 | NM_015436 | chr4 | q |
| 34 | RCHY1 | NM_001008925 | chr4 | q |
| 34 | G3BP2 | NM_203504 | chr4 | q |
| 34 | G3BP2 | NM_203505 | chr4 | q |
| 34 | G3BP2 | NM_012297 | chr4 | q |
| 34 | USO1 | NM_003715 | chr4 | q |
| 34 | SDAD1 | NM_018115 | chr4 | q |
| 34 | NUP54 | NM_017426 | chr4 | q |
| 34 | SCARB2 | NM_005506 | chr4 | q |
| 34 | CXCL13 | NM_006419 | chr4 | q |
| 34 | RASGEF1B | NM_152545 | chr4 | q |
| 34 | SEC31A | NM_014933 | chr4 | q |
| 34 | SEC31A | NM_001077208 | chr4 | q |
| 34 | SEC31A | NM_001077206 | chr4 | q |
| 34 | SEC31A | NM_001077207 | chr4 | q |
| 34 | SEC31A | NM_001191049 | chr4 | q |
| 34 | SEC31A | NM_016211 | chr4 | q |
| 35 | KIAA1310 | NM_017991 | chr2 | q |
| 35 | KIAA1310 | NM_001115016 | chr2 | q |
| 35 | UNC50 | NM_014044 | chr2 | q |
| 35 | TSGA10 | NM_025244 | chr2 | q |
| 35 | TSGA10 | NM_182911 | chr2 | q |
| 35 | LIPT1 | NM_145197 | chr2 | q |
| 35 | LIPT1 | NM_145196 | chr2 | q |
| 35 | LIPT1 | NM_015929 | chr2 | q |
| 35 | LIPT1 | NM_145198 | chr2 | q |
| 35 | LIPT1 | NM_145199 | chr2 | q |
| 35 | AFF3 | NM_001025108 | chr2 | q |
| 35 | AFF3 | NM_002285 | chr2 | q |
| 35 | NPHP1 | NM_000272 | chr2 | q |
| 35 | NPHP1 | NM_207181 | chr2 | q |
| 35 | NPHP1 | NM_001128179 | chr2 | q |
| 35 | NPHP1 | NM_001128178 | chr2 | q |
| 36 | FAM35A | NM_019054 | chr10 | q |
| 36 | ATAD1 | NM_032810 | chr10 | q |
| 36 | FLJ37201 | NR_026835 | chr10 | q |
| 36 | RPP30 | NM_001104546 | chr10 | q |
| 36 | RPP30 | NM_006413 | chr10 | q |
| 36 | LOC100188947 | NR_024467 | chr10 | q |
| 36 | PPP1R3C | NM_005398 | chr10 | q |
| 37 | FANCI | NM_001113378 | chr15 | q |
| 37 | FANCI | NM_018193 | chr15 | q |
| 37 | LOC254559 | NR_015411 | chr15 | q |
| 37 | RHCG | NM_016321 | chr15 | q |
| 37 | C15orf42 | NM_152259 | chr15 | q |
| 37 | ZNF710 | NM_198526 | chr15 | q |
| 37 | IDH2 | NM_002168 | chr15 | q |
| 37 | SEMA4B | NM_198925 | chr15 | q |
| 37 | SEMA4B | NM_020210 | chr15 | q |
| 37 | CIB1 | NM_006384 | chr15 | q |
| 37 | NGRN | NM_001033088 | chr15 | q |
| 37 | NGRN | NR_028052 | chr15 | q |
| 37 | BLM | NM_000057 | chr15 | q |
| 37 | PRC1 | NM_003981 | chr15 | q |
| 37 | PRC1 | NM_199414 | chr15 | q |
| 37 | PRC1 | NM_199413 | chr15 | q |
| 38 | ARPP21 | NM_001025069 | chr3 | p |
| 38 | ARPP21 | NM_016300 | chr3 | p |
| 38 | ARPP21 | NM_198399 | chr3 | p |
| 38 | ARPP21 | NM_001025068 | chr3 | p |
| 38 | VILL | NM_015873 | chr3 | p |
| 38 | DLEC1 | NM_007335 | chr3 | p |
| 38 | DLEC1 | NM_007337 | chr3 | p |
| 38 | CX3CR1 | NM_001171171 | chr3 | p |
| 38 | CX3CR1 | NM_001171172 | chr3 | p |
| 38 | CX3CR1 | NM_001171174 | chr3 | p |
| 38 | CX3CR1 | NM_001337 | chr3 | p |
| 38 | ZNF619 | NM_001145093 | chr3 | p |
| 38 | ZNF619 | NR_026746 | chr3 | p |
| 38 | ZNF619 | NM_001145082 | chr3 | p |
| 38 | ZNF619 | NM_001145083 | chr3 | p |
| 38 | ULK4 | NM_017886 | chr3 | p |
| 38 | TRAK1 | NM_001042646 | chr3 | p |
| 38 | TRAK1 | NM_014965 | chr3 | p |
| 39 | TGFBR3 | NM_001195684 | chr1 | p |
| 39 | TGFBR3 | NM_001195683 | chr1 | p |
| 39 | TGFBR3 | NM_003243 | chr1 | p |
| 39 | TGFBR3 | NR_036634 | chr1 | p |
| 39 | C1orf146 | NM_001012425 | chr1 | p |
| 39 | BCAR3 | NM_003567 | chr1 | p |
| 39 | LOC100129046 | NR_034091 | chr1 | p |
| 39 | ALG14 | NM_144988 | chr1 | p |
| 39 | CCDC76 | NM_019083 | chr1 | p |
| 39 | AMY2B | NM_020978 | chr1 | p |
| 39 | KIAA1324 | NM_020775 | chr1 | p |
| 39 | MYBPHL | NM_001010985 | chr1 | p |
| 39 | GSTM4 | NR_024538 | chr1 | p |
| 39 | GSTM4 | NM_147148 | chr1 | p |
| 39 | GSTM4 | NM_000850 | chr1 | p |
| 39 | GSTM2 | NM_001142368 | chr1 | p |
| 39 | GSTM2 | NM_000848 | chr1 | p |
| 39 | GSTM3 | NR_024537 | chr1 | p |
| 39 | GSTM3 | NM_000849 | chr1 | p |
| 40 | HEATR2 | NM_017802 | chr7 | p |
| 40 | SUN1 | NM_001171945 | chr7 | p |
| 40 | SUN1 | NM_001171946 | chr7 | p |
| 40 | SUN1 | NM_001171944 | chr7 | p |
| 40 | SUN1 | NM_025154 | chr7 | p |
| 40 | SUN1 | NM_001130965 | chr7 | p |
| 40 | INTS1 | NM_001080453 | chr7 | p |
| 40 | PSMG3 | NM_001134340 | chr7 | p |
| 40 | PSMG3 | NM_032302 | chr7 | p |
| 40 | MAD1L1 | NM_003550 | chr7 | p |
| 40 | MAD1L1 | NM_001013836 | chr7 | p |
| 40 | MAD1L1 | NM_001013837 | chr7 | p |
| 40 | EIF3B | NM_003751 | chr7 | p |
| 40 | EIF3B | NM_001037283 | chr7 | p |
| 40 | KDELR2 | NM_001100603 | chr7 | p |
| 40 | KDELR2 | NM_006854 | chr7 | p |
| 41 | MSRA | NM_001135671 | chr8 | p |
| 41 | MSRA | NM_001135670 | chr8 | p |
| 41 | MSRA | NM_012331 | chr8 | p |
| 41 | MSRA | NM_001199729 | chr8 | p |
| 41 | FAM66A | NR_026789 | chr8 | p |
| 41 | FGF20 | NM_019851 | chr8 | p |
| 41 | PCM1 | NM_006197 | chr8 | p |
| 41 | SORBS3 | NM_001018003 | chr8 | p |
| 41 | SORBS3 | NM_005775 | chr8 | p |
| 41 | NEFL | NM_006158 | chr8 | p |
| 42 | ARPC5 | NM_005717 | chr1 | q |
| 42 | IVNS1ABP | NM_006469 | chr1 | q |
| 42 | FAM5C | NM_199051 | chr1 | q |
| 42 | UCHL5 | NM_015984 | chr1 | q |
| 42 | UCHL5 | NM_001199261 | chr1 | q |
| 42 | UCHL5 | NM_001199262 | chr1 | q |
| 42 | UCHL5 | NM_001199263 | chr1 | q |
| 42 | CDC73 | NM_024529 | chr1 | q |
| 42 | CFHR5 | NM_030787 | chr1 | q |
| 42 | ASPM | NM_018136 | chr1 | q |
| 42 | KIF14 | NM_014875 | chr1 | q |
| 42 | C1orf106 | NM_018265 | chr1 | q |
| 42 | C1orf106 | NM_001142569 | chr1 | q |
| 42 | LAD1 | NM_005558 | chr1 | q |
| 42 | CHI3L1 | NM_001276 | chr1 | q |
| 42 | NEK2 | NM_002497 | chr1 | q |
| 42 | CENPF | NM_016343 | chr1 | q |
| 42 | EPRS | NM_004446 | chr1 | q |
| 42 | IARS2 | NM_018060 | chr1 | q |
| 42 | RAB3GAP2 | NM_012414 | chr1 | q |
| 42 | AIDA | NM_022831 | chr1 | q |
| 42 | WDR26 | NM_025160 | chr1 | q |
| 42 | WDR26 | NM_001115113 | chr1 | q |
| 42 | SRP9 | NM_001130440 | chr1 | q |
| 42 | SRP9 | NM_003133 | chr1 | q |
| 42 | ACBD3 | NM_022735 | chr1 | q |
| 42 | LIN9 | NM_173083 | chr1 | q |
| 42 | GUK1 | NM_000858 | chr1 | q |
| 42 | GUK1 | NM_001159391 | chr1 | q |
| 42 | GUK1 | NM_001159390 | chr1 | q |
| 42 | C1orf198 | NM_032800 | chr1 | q |
| 42 | C1orf198 | NM_001136495 | chr1 | q |
| 42 | C1orf198 | NM_001136494 | chr1 | q |
| 42 | EGLN1 | NM_022051 | chr1 | q |
| 42 | SIPA1L2 | NM_020808 | chr1 | q |
| 42 | IRF2BP2 | NM_001077397 | chr1 | q |
| 42 | IRF2BP2 | NM_182972 | chr1 | q |
| 42 | RBM34 | NM_001161533 | chr1 | q |
| 42 | RBM34 | NM_015014 | chr1 | q |
| 42 | RBM34 | NR_027762 | chr1 | q |
| 42 | B3GALNT2 | NM_152490 | chr1 | q |
| 42 | CHRM3 | NM_000740 | chr1 | q |
| 42 | KMO | NM_003679 | chr1 | q |
| 42 | OPN3 | NM_014322 | chr1 | q |
| 42 | EXO1 | NM_003686 | chr1 | q |
| 42 | EXO1 | NM_006027 | chr1 | q |
| 42 | EXO1 | NM_130398 | chr1 | q |
| 42 | PPPDE1 | NM_016076 | chr1 | q |
| 42 | AHCTF1 | NM_015446 | chr1 | q |
| 42 | ZNF695 | NM_020394 | chr1 | q |
| 43 | POLQ | NM_199420 | chr3 | q |
| 43 | PDIA5 | NM_006810 | chr3 | q |
| 43 | PDIA5 | NR_028444 | chr3 | q |
| 43 | PTPLB | NM_198402 | chr3 | q |
| 43 | ALG1L | NM_001015050 | chr3 | q |
| 43 | ALG1L | NM_001195223 | chr3 | q |
| 43 | PODXL2 | NM_015720 | chr3 | q |
| 43 | SEC61A1 | NM_013336 | chr3 | q |
| 44 | RAPGEF5 | NM_012294 | chr7 | p |
| 44 | MGC87042 | NM_207342 | chr7 | p |
| 44 | MGC87042 | NM_001164460 | chr7 | p |
| 44 | TOMM7 | NM_019059 | chr7 | p |
| 44 | C7orf30 | NM_138446 | chr7 | p |
| 44 | NPY | NM_000905 | chr7 | p |
| 44 | MPP6 | NM_016447 | chr7 | p |
| 44 | DFNA5 | NM_004403 | chr7 | p |
| 44 | DFNA5 | NM_001127453 | chr7 | p |
| 44 | DFNA5 | NM_001127454 | chr7 | p |
| 44 | CYCS | NM_018947 | chr7 | p |
| 44 | HNRNPA2B1 | NM_002137 | chr7 | p |
| 44 | HNRNPA2B1 | NM_031243 | chr7 | p |
| 44 | CBX3 | NM_007276 | chr7 | p |
| 44 | CBX3 | NM_016587 | chr7 | p |
| 45 | CNGB3 | NM_019098 | chr8 | q |
| 45 | RIPK2 | NM_003821 | chr8 | q |
| 45 | LRRC69 | NM_001129890 | chr8 | q |
| 45 | RAD54B | NM_012415 | chr8 | q |
| 45 | ESRP1 | NM_001122827 | chr8 | q |
| 45 | ESRP1 | NM_017697 | chr8 | q |
| 45 | ESRP1 | NM_001122825 | chr8 | q |
| 45 | ESRP1 | NM_001122826 | chr8 | q |
| 45 | ESRP1 | NM_001034915 | chr8 | q |
| 45 | UQCRB | NM_006294 | chr8 | q |
| 45 | UQCRB | NM_001199975 | chr8 | q |
| 45 | PTDSS1 | NM_014754 | chr8 | q |
| 45 | STK3 | NM_006281 | chr8 | q |
| 45 | RNF19A | NM_015435 | chr8 | q |
| 45 | RNF19A | NM_183419 | chr8 | q |
| 45 | PABPC1 | NM_002568 | chr8 | q |
| 45 | YWHAZ | NM_001135699 | chr8 | q |
| 45 | YWHAZ | NM_001135701 | chr8 | q |
| 45 | YWHAZ | NM_001135702 | chr8 | q |
| 45 | YWHAZ | NM_001135700 | chr8 | q |
| 45 | YWHAZ | NM_145690 | chr8 | q |
| 45 | YWHAZ | NM_003406 | chr8 | q |
| 45 | FLJ42969 | NR_033962 | chr8 | q |
| 45 | GRHL2 | NM_024915 | chr8 | q |
| 45 | NCALD | NM_001040629 | chr8 | q |
| 45 | NCALD | NM_001040630 | chr8 | q |
| 45 | NCALD | NM_001040627 | chr8 | q |
| 45 | NCALD | NM_032041 | chr8 | q |
| 45 | NCALD | NM_001040628 | chr8 | q |
| 45 | NCALD | NM_001040626 | chr8 | q |
| 45 | NCALD | NM_001040625 | chr8 | q |
| 45 | NCALD | NM_001040624 | chr8 | q |
| 45 | SLC25A32 | NM_030780 | chr8 | q |
| 45 | LRP12 | NM_001135703 | chr8 | q |
| 45 | LRP12 | NM_013437 | chr8 | q |
| 45 | EIF3E | NM_001568 | chr8 | q |
| 45 | KCNV1 | NM_014379 | chr8 | q |
| 45 | EXT1 | NM_000127 | chr8 | q |
| 45 | TAF2 | NM_003184 | chr8 | q |
| 45 | MTBP | NM_022045 | chr8 | q |
| 45 | WDR67 | NM_145647 | chr8 | q |
| 45 | WDR67 | NM_001145088 | chr8 | q |
| 45 | FAM83A | NM_032899 | chr8 | q |
| 45 | FAM83A | NM_207006 | chr8 | q |
| 45 | SQLE | NM_003129 | chr8 | q |
| 45 | NDRG1 | NM_006096 | chr8 | q |
| 45 | NDRG1 | NM_001135242 | chr8 | q |
| 45 | PTK2 | NM_005607 | chr8 | q |
| 45 | PTK2 | NM_153831 | chr8 | q |
| 45 | PTK2 | NM_001199649 | chr8 | q |
| 45 | SLC45A4 | NM_001080431 | chr8 | q |
| 45 | LYPD2 | NM_205545 | chr8 | q |
| 45 | LY6D | NM_003695 | chr8 | q |
| 45 | C8orf73 | NM_001100878 | chr8 | q |
| 45 | PYCRL | NM_023078 | chr8 | q |
| 45 | TSTA3 | NM_003313 | chr8 | q |
| 45 | LOC100128338 | NR_033849 | chr8 | q |
| 45 | GRINA | NM_000837 | chr8 | q |
| 45 | GRINA | NM_001009184 | chr8 | q |
| 45 | SPATC1 | NM_198572 | chr8 | q |
| 45 | SPATC1 | NM_001134374 | chr8 | q |
| 45 | OPLAH | NM_017570 | chr8 | q |
| 45 | GPAA1 | NM_003801 | chr8 | q |
| 45 | HEATR7A | NM_032450 | chr8 | q |
| 45 | HEATR7A | NM_001099281 | chr8 | q |
| 45 | HEATR7A | NM_001099280 | chr8 | q |
| 45 | ADCK5 | NM_174922 | chr8 | q |
| 45 | CYHR1 | NM_001129888 | chr8 | q |
| 45 | CYHR1 | NM_138496 | chr8 | q |
| 45 | CYHR1 | NM_032687 | chr8 | q |
| 45 | RPL8 | NM_000973 | chr8 | q |
| 45 | RPL8 | NM_033301 | chr8 | q |
| 46 | CIC | NM_015125 | chr19 | q |
| 46 | ZNF576 | NM_024327 | chr19 | q |
| 46 | ZNF576 | NM_001145347 | chr19 | q |
| 46 | CADM4 | NM_145296 | chr19 | q |
| 46 | ZNF221 | NM_013359 | chr19 | q |
| 46 | ZNF230 | NM_006300 | chr19 | q |
| 46 | ZNF224 | NM_013398 | chr19 | q |
| 46 | LOC100379224 | NR_033341 | chr19 | q |
| 46 | ZNF226 | NM_015919 | chr19 | q |
| 46 | ZNF226 | NM_001032373 | chr19 | q |
| 46 | ZNF226 | NM_001146220 | chr19 | q |
| 46 | ZNF226 | NM_001032372 | chr19 | q |
| 46 | ZNF226 | NM_001032374 | chr19 | q |
| 46 | ZNF227 | NM_182490 | chr19 | q |
| 46 | ZFP112 | NM_013380 | chr19 | q |
| 46 | ZFP112 | NM_001083335 | chr19 | q |
| 46 | ZNF285 | NM_152354 | chr19 | q |
| 47 | LDLRAD3 | NM_174902 | chr11 | p |
| 47 | COMMD9 | NM_014186 | chr11 | p |
| 47 | COMMD9 | NM_001101653 | chr11 | p |
| 47 | C11orf74 | NM_138787 | chr11 | p |
| 47 | LRRC4C | NM_020929 | chr11 | p |
| 47 | ALKBH3 | NM_139178 | chr11 | p |
| 47 | AGBL2 | NM_024783 | chr11 | p |
| 48 | SLC10A7 | NM_001029998 | chr4 | q |
| 48 | SLC10A7 | NM_032128 | chr4 | q |
| 48 | FBXW7 | NM_001013415 | chr4 | q |
| 48 | FBXW7 | NM_018315 | chr4 | q |
| 48 | FBXW7 | NM_033632 | chr4 | q |
| 48 | MND1 | NM_032117 | chr4 | q |
| 48 | FGG | NM_021870 | chr4 | q |
| 48 | FGG | NM_000509 | chr4 | q |
| 48 | ACCN5 | NM_017419 | chr4 | q |
| 48 | TDO2 | NM_005651 | chr4 | q |
| 49 | ZNF721 | NM_133474 | chr4 | p |
| 49 | PIGG | NM_001127178 | chr4 | p |
| 49 | PIGG | NM_017733 | chr4 | p |
| 49 | PDE6B | NM_001145291 | chr4 | p |
| 49 | PDE6B | NM_001145292 | chr4 | p |
| 49 | PDE6B | NM_000283 | chr4 | p |
| 49 | MFSD7 | NM_032219 | chr4 | p |
| 49 | PCGF3 | NM_006315 | chr4 | p |
| 49 | CPLX1 | NM_006651 | chr4 | p |
| 49 | TNIP2 | NM_024309 | chr4 | p |
| 49 | TNIP2 | NM_001161527 | chr4 | p |
| 49 | C4orf44 | NM_001042690 | chr4 | p |
| 49 | RGS12 | NM_002926 | chr4 | p |
| 49 | RGS12 | NM_198229 | chr4 | p |
| 49 | RGS12 | NM_198227 | chr4 | p |
| 49 | STK32B | NM_018401 | chr4 | p |
| 49 | CRMP1 | NM_001014809 | chr4 | p |
| 49 | CRMP1 | NM_001313 | chr4 | p |
| 49 | WFS1 | NM_001145853 | chr4 | p |
| 49 | WFS1 | NM_006005 | chr4 | p |
| 49 | LOC100129931 | NR_033828 | chr4 | p |
| 49 | ACOX3 | NM_003501 | chr4 | p |
| 49 | ACOX3 | NM_001101667 | chr4 | p |
| 50 | FLJ10661 | NM_001099653 | chr11 | q |
| 50 | FLJ10661 | NM_018172 | chr11 | q |
| 50 | FLJ10661 | NM_152563 | chr11 | q |
| 50 | LRTOMT | NM_001145307 | chr11 | q |
| 50 | LRTOMT | NM_001145310 | chr11 | q |
| 50 | LRTOMT | NM_001145309 | chr11 | q |
| 50 | LRTOMT | NM_001145308 | chr11 | q |
| 50 | LRTOMT | NM_145309 | chr11 | q |
| 50 | LRTOMT | NR_026886 | chr11 | q |
| 50 | ME3 | NM_001161586 | chr11 | q |
| 50 | ME3 | NM_001014811 | chr11 | q |
| 50 | ME3 | NM_006680 | chr11 | q |
| 50 | KDM4D | NM_018039 | chr11 | q |
| 50 | PGR | NM_000926 | chr11 | q |
| 50 | TRPC6 | NM_004621 | chr11 | q |
| 50 | C11orf70 | NM_032930 | chr11 | q |
| 50 | C11orf70 | NM_001195005 | chr11 | q |
| 50 | ACAT1 | NM_000019 | chr11 | q |
| 50 | FDXACB1 | NM_138378 | chr11 | q |
| 50 | DIXDC1 | NM_001037954 | chr11 | q |
| 50 | DIXDC1 | NM_033425 | chr11 | q |
| 50 | PIH1D2 | NM_001082619 | chr11 | q |
| 50 | PIH1D2 | NM_138789 | chr11 | q |
| 50 | C11orf34 | NM_001145024 | chr11 | q |
| 50 | TTC12 | NM_017868 | chr11 | q |
| 50 | ANKK1 | NM_178510 | chr11 | q |
| 50 | USP28 | NM_020886 | chr11 | q |
| 50 | RNF214 | NM_207343 | chr11 | q |
| 50 | RNF214 | NM_001077239 | chr11 | q |
| 51 | UBXN10 | NM_152376 | chr1 | p |
| 51 | PINK1 | NM_032409 | chr1 | p |
| 51 | NBPF3 | NM_032264 | chr1 | p |
| 51 | HSPC157 | NR_023918 | chr1 | p |
| 51 | HSPC157 | NR_023919 | chr1 | p |
| 51 | ZBTB40 | NM_014870 | chr1 | p |
| 51 | ZBTB40 | NM_001083621 | chr1 | p |
| 51 | TCEA3 | NM_003196 | chr1 | p |
| 51 | HMGCL | NM_000191 | chr1 | p |
| 51 | HMGCL | NM_001166059 | chr1 | p |
| 51 | MYOM3 | NM_152372 | chr1 | p |
| 51 | PAQR7 | NM_178422 | chr1 | p |
| 51 | UBXN11 | NM_183008 | chr1 | p |
| 51 | UBXN11 | NM_145345 | chr1 | p |
| 51 | UBXN11 | NM_001077262 | chr1 | p |
| 51 | WDTC1 | NM_015023 | chr1 | p |
| 51 | SRSF4 | NM_005626 | chr1 | p |
| 51 | BAI2 | NM_001703 | chr1 | p |
| 51 | CCDC28B | NM_024296 | chr1 | p |
| 51 | IQCC | NM_018134 | chr1 | p |
| 51 | IQCC | NM_001160042 | chr1 | p |
| 51 | MTMR9LP | NR_026850 | chr1 | p |
| 51 | ADC | NM_052998 | chr1 | p |
| 51 | TEKT2 | NM_014466 | chr1 | p |
| 51 | MAP7D1 | NM_018067 | chr1 | p |
| 51 | OSCP1 | NM_145047 | chr1 | p |
| 51 | OSCP1 | NM_206837 | chr1 | p |
| 51 | DNALI1 | NM_003462 | chr1 | p |
| 51 | RSPO1 | NM_001038633 | chr1 | p |
| 52 | TEX2 | NM_018469 | chr17 | q |
| 52 | POLG2 | NM_007215 | chr17 | q |
| 52 | DDX5 | NM_004396 | chr17 | q |
| 52 | CCDC45 | NM_138363 | chr17 | q |
| 52 | SMURF2 | NM_022739 | chr17 | q |
| 52 | LOC146880 | NR_027487 | chr17 | q |
| 52 | LOC146880 | NR_026899 | chr17 | q |
| 52 | PLEKHM1P | NR_024386 | chr17 | q |
| 52 | NOL11 | NM_015462 | chr17 | q |
| 52 | BPTF | NM_182641 | chr17 | q |
| 52 | BPTF | NM_004459 | chr17 | q |
| 52 | KPNA2 | NM_002266 | chr17 | q |
| 53 | LOC145837 | NR_026979 | chr15 | q |
| 53 | LRRC49 | NM_017691 | chr15 | q |
| 53 | LRRC49 | NM_001199017 | chr15 | q |
| 53 | LRRC49 | NM_001199018 | chr15 | q |
| 53 | CT62 | NM_001102658 | chr15 | q |
| 53 | CELF6 | NM_052840 | chr15 | q |
| 53 | CELF6 | NM_001172684 | chr15 | q |
| 53 | CELF6 | NM_001172685 | chr15 | q |
| 53 | HEXA | NM_000520 | chr15 | q |
| 53 | LOXL1 | NM_005576 | chr15 | q |
| 54 | PPP1CB | NM_002709 | chr2 | p |
| 54 | PPP1CB | NM_206876 | chr2 | p |
| 54 | GALNT14 | NM_024572 | chr2 | p |
| 54 | MEMO1 | NM_015955 | chr2 | p |
| 54 | MEMO1 | NM_001137602 | chr2 | p |
| 54 | BIRC6 | NM_016252 | chr2 | p |
| 54 | FAM98A | NM_015475 | chr2 | p |
| 54 | ATL2 | NM_022374 | chr2 | p |
| 54 | ATL2 | NM_001135673 | chr2 | p |
| 54 | ATL2 | NR_024191 | chr2 | p |
| 54 | EML4 | NM_001145076 | chr2 | p |
| 54 | EML4 | NM_019063 | chr2 | p |
| 54 | LRPPRC | NM_133259 | chr2 | p |
| 54 | PPM1B | NM_177969 | chr2 | p |
| 54 | PPM1B | NM_001033556 | chr2 | p |
| 54 | PPM1B | NM_177968 | chr2 | p |
| 54 | PPM1B | NM_002706 | chr2 | p |
| 54 | PPM1B | NM_001033557 | chr2 | p |
| 54 | C2orf34 | NM_024766 | chr2 | p |
| 54 | SRBD1 | NM_018079 | chr2 | p |
| 54 | EPCAM | NM_002354 | chr2 | p |
| 54 | PSME4 | NM_014614 | chr2 | p |
| 55 | ZBTB1 | NM_001123329 | chr14 | q |
| 55 | ZBTB1 | NM_014950 | chr14 | q |
| 55 | C14orf50 | NM_172365 | chr14 | q |
| 55 | TMEM229B | NM_182526 | chr14 | q |
| 55 | EXD2 | NM_001193360 | chr14 | q |
| 55 | EXD2 | NR_034165 | chr14 | q |
| 55 | EXD2 | NR_034164 | chr14 | q |
| 55 | EXD2 | NM_001193361 | chr14 | q |
| 55 | EXD2 | NM_018199 | chr14 | q |
| 55 | EXD2 | NM_001193363 | chr14 | q |
| 55 | EXD2 | NM_001193362 | chr14 | q |
| 55 | GALNTL1 | NM_001168368 | chr14 | q |
| 55 | GALNTL1 | NM_020692 | chr14 | q |
| 55 | UPF0639 | NM_001161498 | chr14 | q |
| 55 | SRSF5 | NM_006925 | chr14 | q |
| 55 | SRSF5 | NM_001039465 | chr14 | q |
| 55 | DNAL1 | NM_031427 | chr14 | q |
| 55 | C14orf45 | NM_025057 | chr14 | q |
| 55 | EIF2B2 | NM_014239 | chr14 | q |
| 55 | KIAA1737 | NM_033426 | chr14 | q |
| 55 | POMT2 | NM_013382 | chr14 | q |
| 55 | STON2 | NM_033104 | chr14 | q |
| 55 | GALC | NM_000153 | chr14 | q |
| 55 | GALC | NM_001037525 | chr14 | q |
| 55 | TTC8 | NM_198309 | chr14 | q |
| 55 | TTC8 | NM_198310 | chr14 | q |
| 55 | TTC8 | NM_144596 | chr14 | q |
| 55 | KCNK13 | NM_022054 | chr14 | q |
| 55 | ATXN3 | NR_028457 | chr14 | q |
| 55 | ATXN3 | NR_028455 | chr14 | q |
| 55 | ATXN3 | NR_028462 | chr14 | q |
| 55 | ATXN3 | NR_028461 | chr14 | q |
| 55 | ATXN3 | NM_030660 | chr14 | q |
| 55 | ATXN3 | NM_001164774 | chr14 | q |
| 55 | ATXN3 | NM_001164778 | chr14 | q |
| 55 | ATXN3 | NM_001164781 | chr14 | q |
| 55 | ATXN3 | NM_001164782 | chr14 | q |
| 55 | ATXN3 | NM_001164777 | chr14 | q |
| 55 | ATXN3 | NM_001164780 | chr14 | q |
| 55 | ATXN3 | NM_001164779 | chr14 | q |
| 55 | ATXN3 | NR_028470 | chr14 | q |
| 55 | ATXN3 | NR_028469 | chr14 | q |
| 55 | ATXN3 | NM_001127697 | chr14 | q |
| 55 | ATXN3 | NR_028468 | chr14 | q |
| 55 | ATXN3 | NR_028467 | chr14 | q |
| 55 | ATXN3 | NR_028466 | chr14 | q |
| 55 | ATXN3 | NR_028465 | chr14 | q |
| 55 | ATXN3 | NM_004993 | chr14 | q |
| 55 | ATXN3 | NR_028460 | chr14 | q |
| 55 | ATXN3 | NR_028459 | chr14 | q |
| 55 | ATXN3 | NR_028458 | chr14 | q |
| 55 | ATXN3 | NM_001164776 | chr14 | q |
| 55 | ATXN3 | NR_028453 | chr14 | q |
| 55 | ATXN3 | NR_028456 | chr14 | q |
| 55 | ATXN3 | NM_001127696 | chr14 | q |
| 55 | ATXN3 | NR_028454 | chr14 | q |
| 55 | ATXN3 | NR_028464 | chr14 | q |
| 55 | ATXN3 | NR_031765 | chr14 | q |
| 55 | ATXN3 | NR_028463 | chr14 | q |
| 56 | NSUN2 | NM_001193455 | chr5 | p |
| 56 | NSUN2 | NM_017755 | chr5 | p |
| 56 | SRD5A1 | NM_001047 | chr5 | p |
| 56 | PAPD7 | NM_006999 | chr5 | p |
| 56 | PAPD7 | NM_001171806 | chr5 | p |
| 56 | PAPD7 | NM_001171805 | chr5 | p |
| 56 | MTRR | NM_024010 | chr5 | p |
| 56 | MTRR | NM_002454 | chr5 | p |
| 56 | CCT5 | NM_012073 | chr5 | p |
| 56 | MARCH6 | NM_005885 | chr5 | p |
| 56 | DAP | NM_004394 | chr5 | p |
| 56 | DROSHA | NM_013235 | chr5 | p |
| 56 | DROSHA | NM_001100412 | chr5 | p |
| 56 | C5orf22 | NM_018356 | chr5 | p |
| 56 | GOLPH3 | NM_022130 | chr5 | p |
| 56 | ZFR | NM_016107 | chr5 | p |
| 56 | TARS | NM_152295 | chr5 | p |
| 57 | PRR11 | NM_018304 | chr17 | q |
| 57 | CLTC | NM_004859 | chr17 | q |
| 57 | PTRH2 | NM_016077 | chr17 | q |
| 57 | TMEM49 | NM_030938 | chr17 | q |
| 57 | HEATR6 | NM_022070 | chr17 | q |
| 57 | C17orf64 | NM_181707 | chr17 | q |
| 57 | BCAS3 | NM_001099432 | chr17 | q |
| 57 | BCAS3 | NM_017679 | chr17 | q |
| 57 | TBX4 | NM_018488 | chr17 | q |
| 57 | MED13 | NM_005121 | chr17 | q |
| 57 | DCAF7 | NM_005828 | chr17 | q |
| 57 | STRADA | NM_001003786 | chr17 | q |
| 57 | STRADA | NM_001003787 | chr17 | q |
| 57 | STRADA | NM_001165969 | chr17 | q |
| 57 | STRADA | NM_153335 | chr17 | q |
| 57 | STRADA | NM_001165970 | chr17 | q |
| 57 | STRADA | NM_001003788 | chr17 | q |
| 57 | CCDC47 | NM_020198 | chr17 | q |
| 57 | DDX42 | NM_007372 | chr17 | q |
| 57 | DDX42 | NM_203499 | chr17 | q |
| 57 | FTSJ3 | NM_017647 | chr17 | q |
| 57 | PSMC5 | NM_002805 | chr17 | q |
| 57 | PSMC5 | NM_001199163 | chr17 | q |
| 57 | SMARCD2 | NM_001098426 | chr17 | q |
| 57 | TCAM1P | NR_002947 | chr17 | q |
| 58 | KIAA1609 | NM_020947 | chr16 | q |
| 58 | USP10 | NM_005153 | chr16 | q |
| 58 | KIAA0182 | NM_001134473 | chr16 | q |
| 58 | KIAA0182 | NM_014615 | chr16 | q |
| 58 | COX4I1 | NM_001861 | chr16 | q |
| 58 | JPH3 | NM_020655 | chr16 | q |
| 58 | SLC7A5 | NM_003486 | chr16 | q |
| 58 | BANP | NM_001173543 | chr16 | q |
| 58 | BANP | NM_079837 | chr16 | q |
| 58 | BANP | NM_017869 | chr16 | q |
| 58 | BANP | NM_001173542 | chr16 | q |
| 58 | BANP | NM_001173541 | chr16 | q |
| 58 | BANP | NM_001173539 | chr16 | q |
| 58 | BANP | NM_001173540 | chr16 | q |
| 58 | ZC3H18 | NM_144604 | chr16 | q |
| 58 | MVD | NM_002461 | chr16 | q |
| 58 | ANKRD11 | NM_013275 | chr16 | q |
| 58 | CHMP1A | NM_002768 | chr16 | q |
| 58 | CHMP1A | NM_001083314 | chr16 | q |
| 58 | LOC100128881 | NR_036480 | chr16 | q |
| 58 | FANCA | NM_000135 | chr16 | q |
| 58 | FANCA | NM_001018112 | chr16 | q |
| 58 | DEF8 | NM_207514 | chr16 | q |
| 58 | DEF8 | NM_017702 | chr16 | q |
| 58 | DBNDD1 | NM_001042610 | chr16 | q |
| 58 | DBNDD1 | NM_024043 | chr16 | q |
| 59 | PHIP | NM_017934 | chr6 | q |
| 59 | TTK | NM_001166691 | chr6 | q |
| 59 | TTK | NM_003318 | chr6 | q |
| 59 | ME1 | NM_002395 | chr6 | q |
| 59 | RIPPLY2 | NM_001009994 | chr6 | q |
| 59 | ATG5 | NM_004849 | chr6 | q |
| 59 | PDSS2 | NM_020381 | chr6 | q |
| 60 | KIF4A | NM_012310 | chrX | q |
| 60 | RLIM | NM_183353 | chrX | q |
| 60 | RLIM | NM_016120 | chrX | q |
| 60 | MAGT1 | NM_032121 | chrX | q |
| 60 | COX7B | NM_001866 | chrX | q |
| 60 | PGK1 | NM_000291 | chrX | q |
| 60 | TAF9B | NM_015975 | chrX | q |
| 60 | PCDH11X | NM_032968 | chrX | q |
| 60 | PCDH11X | NM_001168360 | chrX | q |
| 60 | PCDH11X | NM_032969 | chrX | q |
| 60 | PCDH11X | NM_032967 | chrX | q |
| 60 | PCDH11X | NM_001168363 | chrX | q |
| 60 | PCDH11X | NM_001168362 | chrX | q |
| 60 | PCDH11X | NM_001168361 | chrX | q |
| 60 | PCDH11X | NM_014522 | chrX | q |
| 61 | FLYWCH2 | NM_001142500 | chr16 | p |
| 61 | FLYWCH2 | NM_001142499 | chr16 | p |
| 61 | FLYWCH2 | NM_138439 | chr16 | p |
| 61 | MMP25 | NM_022468 | chr16 | p |
| 61 | MGC3771 | NR_024166 | chr16 | p |
| 61 | MGC3771 | NR_024167 | chr16 | p |
| 61 | ZNF263 | NM_005741 | chr16 | p |
| 61 | TIGD7 | NM_033208 | chr16 | p |
| 61 | ZNF434 | NM_017810 | chr16 | p |
| 61 | ZNF174 | NM_001032292 | chr16 | p |
| 61 | ZNF174 | NM_003450 | chr16 | p |
| 61 | ZNF597 | NM_152457 | chr16 | p |
| 61 | NAT15 | NM_001083601 | chr16 | p |
| 61 | NAT15 | NM_024845 | chr16 | p |
| 61 | NAT15 | NM_001083600 | chr16 | p |
| 61 | CLUAP1 | NM_015041 | chr16 | p |
| 61 | CLUAP1 | NM_024793 | chr16 | p |
| 61 | CREBBP | NM_004380 | chr16 | p |
| 61 | CREBBP | NM_001079846 | chr16 | p |
| 61 | GLIS2 | NM_032575 | chr16 | p |
| 61 | C16orf5 | NM_013399 | chr16 | p |
| 61 | C16orf5 | NM_001199054 | chr16 | p |
| 61 | C16orf5 | NM_001199055 | chr16 | p |
| 61 | C16orf5 | NM_001199056 | chr16 | p |
| 61 | MGRN1 | NM_001142290 | chr16 | p |
| 61 | MGRN1 | NM_015246 | chr16 | p |
| 61 | MGRN1 | NM_001142289 | chr16 | p |
| 61 | MGRN1 | NM_001142291 | chr16 | p |
| 61 | C16orf71 | NM_139170 | chr16 | p |
| 61 | NAGPA | NM_016256 | chr16 | p |
| 61 | ABAT | NM_000663 | chr16 | p |
| 61 | ABAT | NM_020686 | chr16 | p |
| 61 | ABAT | NM_001127448 | chr16 | p |
| 61 | NUBP1 | NM_002484 | chr16 | p |
| 61 | CLEC16A | NM_015226 | chr16 | p |
| 61 | MKL2 | NM_014048 | chr16 | p |
| 61 | NOMO2 | NM_001004060 | chr16 | p |
| 61 | NOMO2 | NM_173614 | chr16 | p |
| 61 | ACSM5 | NM_017888 | chr16 | p |
| 61 | LYRM1 | NM_020424 | chr16 | p |
| 61 | LYRM1 | NM_001128301 | chr16 | p |
| 61 | LYRM1 | NM_001128302 | chr16 | p |
| 61 | C16orf52 | NM_001164579 | chr16 | p |
| 61 | GGA2 | NM_015044 | chr16 | p |
| 61 | RBBP6 | NM_006910 | chr16 | p |
| 61 | RBBP6 | NM_032626 | chr16 | p |
| 61 | RBBP6 | NM_018703 | chr16 | p |
| 61 | AQP8 | NM_001169 | chr16 | p |
| 61 | NSMCE1 | NM_145080 | chr16 | p |
| 61 | FLJ21408 | NR_037184 | chr16 | p |
| 61 | KIAA0556 | NM_015202 | chr16 | p |
| 61 | CCDC101 | NM_138414 | chr16 | p |
| 61 | MVP | NM_005115 | chr16 | p |
| 61 | MVP | NM_017458 | chr16 | p |
| 61 | YPEL3 | NM_001145524 | chr16 | p |
| 61 | YPEL3 | NM_031477 | chr16 | p |
| 61 | LOC595101 | NR_002453 | chr16 | p |
| 61 | TBC1D10B | NM_015527 | chr16 | p |
| 61 | SEPT1 | NM_052838 | chr16 | p |
| 61 | ZNF747 | NM_023931 | chr16 | p |
| 61 | PHKG2 | NM_001172432 | chr16 | p |
| 61 | PHKG2 | NM_000294 | chr16 | p |
| 61 | BCL7C | NM_004765 | chr16 | p |
| 61 | HSD3B7 | NM_001142778 | chr16 | p |
| 61 | HSD3B7 | NM_001142777 | chr16 | p |
| 61 | HSD3B7 | NM_025193 | chr16 | p |
| 61 | STX1B | NM_052874 | chr16 | p |
| 61 | ZNF668 | NM_024706 | chr16 | p |
| 61 | ZNF668 | NM_001172670 | chr16 | p |
| 61 | ZNF668 | NM_001172669 | chr16 | p |
| 61 | ZNF668 | NM_001172668 | chr16 | p |
| 61 | ZNF646 | NM_014699 | chr16 | p |
| 61 | ZNF843 | NM_001136509 | chr16 | p |
| 61 | ARMC5 | NM_024742 | chr16 | p |
| 61 | ARMC5 | NM_001105247 | chr16 | p |
| 61 | TGFB1I1 | NM_001164719 | chr16 | p |
| 61 | TGFB1I1 | NM_015927 | chr16 | p |
| 61 | TGFB1I1 | NM_001042454 | chr16 | p |
| 61 | C16orf58 | NM_022744 | chr16 | p |
| 62 | NUP93 | NM_014669 | chr16 | q |
| 62 | HERPUD1 | NM_001010990 | chr16 | q |
| 62 | HERPUD1 | NM_001010989 | chr16 | q |
| 62 | HERPUD1 | NM_014685 | chr16 | q |
| 62 | COQ9 | NM_020312 | chr16 | q |
| 62 | GPR56 | NM_001145774 | chr16 | q |
| 62 | GPR56 | NM_201524 | chr16 | q |
| 62 | GPR56 | NM_005682 | chr16 | q |
| 62 | GPR56 | NM_001145771 | chr16 | q |
| 62 | GPR56 | NM_001145770 | chr16 | q |
| 62 | GPR56 | NM_001145773 | chr16 | q |
| 62 | GPR56 | NM_001145772 | chr16 | q |
| 62 | GPR56 | NM_201525 | chr16 | q |
| 62 | MMP15 | NM_002428 | chr16 | q |
| 62 | CNOT1 | NM_016284 | chr16 | q |
| 62 | CNOT1 | NM_206999 | chr16 | q |
| 62 | CDH8 | NM_001796 | chr16 | q |
| 62 | DYNC1LI2 | NM_006141 | chr16 | q |
| 62 | CBFB | NM_022845 | chr16 | q |
| 62 | CBFB | NM_001755 | chr16 | q |
| 62 | GFOD2 | NM_030819 | chr16 | q |
| 62 | GFOD2 | NR_027398 | chr16 | q |
| 62 | GFOD2 | NR_027399 | chr16 | q |
| 62 | RANBP10 | NM_020850 | chr16 | q |
| 62 | NUTF2 | NM_005796 | chr16 | q |
| 62 | DUS2L | NM_017803 | chr16 | q |
| 62 | ESRP2 | NM_024939 | chr16 | q |
| 62 | CYB5B | NM_030579 | chr16 | q |
| 62 | WWP2 | NM_199424 | chr16 | q |
| 62 | WWP2 | NM_007014 | chr16 | q |
| 62 | WWP2 | NM_199423 | chr16 | q |
| 62 | PDXDC2P | NR_003610 | chr16 | q |
| 62 | AARS | NM_001605 | chr16 | q |
| 62 | DDX19A | NM_018332 | chr16 | q |
| 62 | COG4 | NM_001195139 | chr16 | q |
| 62 | COG4 | NM_015386 | chr16 | q |
| 62 | SF3B3 | NM_012426 | chr16 | q |
| 62 | MTSS1L | NM_138383 | chr16 | q |
| 63 | ZNF554 | NM_001102651 | chr19 | p |
| 63 | PIAS4 | NM_015897 | chr19 | p |
| 63 | SIRT6 | NM_016539 | chr19 | p |
| 63 | SIRT6 | NM_001193285 | chr19 | p |
| 63 | ANKRD24 | NM_133475 | chr19 | p |
| 63 | CCDC94 | NM_018074 | chr19 | p |
| 63 | HDGFRP2 | NM_001001520 | chr19 | p |
| 63 | HDGFRP2 | NM_032631 | chr19 | p |
| 63 | KDM4B | NM_015015 | chr19 | p |
| 63 | DUS3L | NM_020175 | chr19 | p |
| 63 | DUS3L | NM_001161619 | chr19 | p |
| 63 | RANBP3 | NM_007322 | chr19 | p |
| 63 | RANBP3 | NM_003624 | chr19 | p |
| 63 | RANBP3 | NM_007320 | chr19 | p |
| 63 | ACSBG2 | NM_030924 | chr19 | p |
| 63 | MLLT1 | NM_005934 | chr19 | p |
| 63 | TRIP10 | NM_004240 | chr19 | p |
| 64 | TMEM169 | NM_138390 | chr2 | q |
| 64 | TMEM169 | NM_001142311 | chr2 | q |
| 64 | TMEM169 | NM_001142312 | chr2 | q |
| 64 | TMEM169 | NM_001142310 | chr2 | q |
| 64 | CTDSP1 | NM_021198 | chr2 | q |
| 64 | CTDSP1 | NM_182642 | chr2 | q |
| 64 | PLCD4 | NM_032726 | chr2 | q |
| 64 | RNF25 | NM_022453 | chr2 | q |
| 64 | STK36 | NM_015690 | chr2 | q |
| 64 | ANKZF1 | NM_018089 | chr2 | q |
| 64 | ANKZF1 | NM_001042410 | chr2 | q |
| 65 | C17orf97 | NM_001013672 | chr17 | p |
| 65 | RNMTL1 | NM_018146 | chr17 | p |
| 65 | ABR | NM_001092 | chr17 | p |
| 65 | ABR | NM_001159746 | chr17 | p |
| 65 | ABR | NM_021962 | chr17 | p |
| 65 | YWHAE | NM_006761 | chr17 | p |
| 65 | YWHAE | NR_024058 | chr17 | p |
| 65 | MYO1C | NM_033375 | chr17 | p |
| 65 | MYO1C | NM_001080950 | chr17 | p |
| 65 | MYO1C | NM_001080779 | chr17 | p |
| 65 | INPP5K | NM_001135642 | chr17 | p |
| 65 | INPP5K | NM_130766 | chr17 | p |
| 65 | INPP5K | NM_016532 | chr17 | p |
| 65 | SCARF1 | NM_145352 | chr17 | p |
| 65 | SCARF1 | NM_145350 | chr17 | p |
| 65 | SCARF1 | NM_003693 | chr17 | p |
| 65 | SCARF1 | NR_028076 | chr17 | p |
| 65 | SCARF1 | NR_028075 | chr17 | p |
| 65 | RTN4RL1 | NM_178568 | chr17 | p |
| 65 | SMG6 | NM_017575 | chr17 | p |
| 65 | SMG6 | NM_001170957 | chr17 | p |
| 65 | SRR | NM_021947 | chr17 | p |
| 65 | SGSM2 | NM_014853 | chr17 | p |
| 65 | SGSM2 | NM_001098509 | chr17 | p |
| 65 | METT10D | NM_024086 | chr17 | p |
| 65 | TRPV1 | NM_080706 | chr17 | p |
| 65 | TRPV1 | NM_080705 | chr17 | p |
| 65 | TRPV1 | NM_018727 | chr17 | p |
| 65 | TRPV1 | NM_080704 | chr17 | p |
| 65 | SHPK | NM_013276 | chr17 | p |
| 65 | C17orf85 | NM_001114118 | chr17 | p |
| 65 | C17orf85 | NM_018553 | chr17 | p |
| 65 | ZZEF1 | NM_015113 | chr17 | p |
| 65 | CYB5D2 | NR_023346 | chr17 | p |
| 65 | CYB5D2 | NR_023347 | chr17 | p |
| 65 | CYB5D2 | NM_144611 | chr17 | p |
| 65 | PLD2 | NM_002663 | chr17 | p |
| 65 | MINK1 | NM_015716 | chr17 | p |
| 65 | MINK1 | NM_153827 | chr17 | p |
| 65 | MINK1 | NM_170663 | chr17 | p |
| 65 | MINK1 | NM_001024937 | chr17 | p |
| 65 | CHRNE | NM_000080 | chr17 | p |
| 65 | ENO3 | NM_053013 | chr17 | p |
| 65 | ENO3 | NM_001976 | chr17 | p |
| 65 | ENO3 | NM_001193503 | chr17 | p |
| 65 | CAMTA2 | NM_001171166 | chr17 | p |
| 65 | CAMTA2 | NM_001171168 | chr17 | p |
| 65 | CAMTA2 | NM_001171167 | chr17 | p |
| 65 | CAMTA2 | NM_015099 | chr17 | p |
| 65 | INCA1 | NM_001167987 | chr17 | p |
| 65 | INCA1 | NM_001167986 | chr17 | p |
| 65 | INCA1 | NM_001167985 | chr17 | p |
| 65 | INCA1 | NM_213726 | chr17 | p |
| 65 | ZFP3 | NM_153018 | chr17 | p |
| 65 | ZNF594 | NM_032530 | chr17 | p |
| 65 | LOC100130950 | NR_034082 | chr17 | p |
| 65 | RABEP1 | NM_001083585 | chr17 | p |
| 65 | RABEP1 | NM_004703 | chr17 | p |
| 65 | RPAIN | NM_001033002 | chr17 | p |
| 65 | RPAIN | NM_001160267 | chr17 | p |
| 65 | RPAIN | NM_001160266 | chr17 | p |
| 65 | RPAIN | NM_001160246 | chr17 | p |
| 65 | RPAIN | NM_001160244 | chr17 | p |
| 65 | RPAIN | NR_027682 | chr17 | p |
| 65 | RPAIN | NR_027679 | chr17 | p |
| 65 | RPAIN | NR_027680 | chr17 | p |
| 65 | RPAIN | NR_027678 | chr17 | p |
| 65 | RPAIN | NR_027681 | chr17 | p |
| 65 | RPAIN | NR_027685 | chr17 | p |
| 65 | RPAIN | NM_001160243 | chr17 | p |
| 65 | RPAIN | NR_027684 | chr17 | p |
| 65 | RPAIN | NR_027683 | chr17 | p |
| 65 | KIAA0753 | NM_014804 | chr17 | p |
| 65 | ALOX12 | NM_000697 | chr17 | p |
| 65 | C17orf49 | NM_001142798 | chr17 | p |
| 65 | C17orf49 | NM_174893 | chr17 | p |
| 65 | C17orf49 | NM_001142799 | chr17 | p |
| 65 | PHF23 | NM_024297 | chr17 | p |
| 65 | GPS2 | NM_004489 | chr17 | p |
| 65 | TNK1 | NM_003985 | chr17 | p |
| 66 | SHCBP1 | NM_024745 | chr16 | q |
| 66 | ORC6 | NM_014321 | chr16 | q |
| 66 | ITFG1 | NM_030790 | chr16 | q |
| 66 | PHKB | NM_000293 | chr16 | q |
| 66 | PHKB | NM_001031835 | chr16 | q |
| 66 | HEATR3 | NM_182922 | chr16 | q |
| 66 | BRD7 | NM_001173984 | chr16 | q |
| 66 | BRD7 | NM_013263 | chr16 | q |
| 66 | NUDT21 | NM_007006 | chr16 | q |
| 67 | C9orf68 | NM_001039395 | chr9 | p |
| 67 | C9orf46 | NM_018465 | chr9 | p |
| 67 | ACER2 | NM_001010887 | chr9 | p |
| 67 | IFT74 | NM_001099222 | chr9 | p |
| 67 | IFT74 | NM_025103 | chr9 | p |
| 67 | IFT74 | NM_001099224 | chr9 | p |
| 67 | IFT74 | NM_001099223 | chr9 | p |
| 67 | NDUFB6 | NM_001199987 | chr9 | p |
| 67 | NDUFB6 | NM_002493 | chr9 | p |
| 67 | NDUFB6 | NM_182739 | chr9 | p |
| 67 | SMU1 | NM_018225 | chr9 | p |
| 67 | DCTN3 | NM_007234 | chr9 | p |
| 67 | DCTN3 | NM_024348 | chr9 | p |
| 68 | BOD1L | NM_148894 | chr4 | p |
| 68 | FAM184B | NM_015688 | chr4 | p |
| 68 | DCAF16 | NM_017741 | chr4 | p |
| 68 | PACRGL | NM_001130727 | chr4 | p |
| 68 | PACRGL | NM_145048 | chr4 | p |
| 68 | SEPSECS | NM_016955 | chr4 | p |
| 68 | ANAPC4 | NM_013367 | chr4 | p |
| 68 | TBC1D19 | NM_018317 | chr4 | p |
| 69 | ATP11B | NM_014616 | chr3 | q |
| 69 | MCCC1 | NM_020166 | chr3 | q |
| 69 | YEATS2 | NM_018023 | chr3 | q |
| 69 | ALG3 | NM_005787 | chr3 | q |
| 69 | ALG3 | NR_024534 | chr3 | q |
| 69 | ALG3 | NR_024533 | chr3 | q |
| 69 | ALG3 | NM_001006941 | chr3 | q |
| 69 | IGF2BP2 | NM_001007225 | chr3 | q |
| 69 | IGF2BP2 | NM_006548 | chr3 | q |
| 69 | RFC4 | NM_002916 | chr3 | q |
| 69 | RFC4 | NM_181573 | chr3 | q |
| 70 | PIK3C3 | NM_002647 | chr18 | q |
| 70 | SETBP1 | NM_015559 | chr18 | q |
| 70 | SETBP1 | NM_001130110 | chr18 | q |
| 70 | SLC14A2 | NM_007163 | chr18 | q |
| 70 | HAUS1 | NR_026978 | chr18 | q |
| 70 | HAUS1 | NM_138443 | chr18 | q |
| 70 | HDHD2 | NM_032124 | chr18 | q |
| 70 | CCDC11 | NM_145020 | chr18 | q |
| 70 | CXXC1 | NM_014593 | chr18 | q |
| 70 | CXXC1 | NM_001101654 | chr18 | q |
| 71 | FANCD2 | NM_001018115 | chr3 | p |
| 71 | FANCD2 | NM_033084 | chr3 | p |
| 71 | C3orf24 | NM_173472 | chr3 | p |
| 71 | C3orf24 | NM_001164839 | chr3 | p |
| 71 | MRPS25 | NM_022497 | chr3 | p |
| 71 | COL6A4P1 | NR_027927 | chr3 | p |
| 71 | SGOL1 | NM_001012411 | chr3 | p |
| 71 | SGOL1 | NM_001199253 | chr3 | p |
| 71 | SGOL1 | NM_001199252 | chr3 | p |
| 71 | SGOL1 | NM_001199251 | chr3 | p |
| 71 | SGOL1 | NM_001012412 | chr3 | p |
| 71 | SGOL1 | NM_001012409 | chr3 | p |
| 71 | SGOL1 | NM_001199254 | chr3 | p |
| 71 | SGOL1 | NM_001012410 | chr3 | p |
| 71 | SGOL1 | NM_138484 | chr3 | p |
| 71 | SGOL1 | NM_001199256 | chr3 | p |
| 71 | SGOL1 | NM_001199257 | chr3 | p |
| 71 | SGOL1 | NM_001012413 | chr3 | p |
| 71 | SGOL1 | NM_001199255 | chr3 | p |
| 71 | UBE2E2 | NM_152653 | chr3 | p |
| 72 | C14orf132 | NR_023938 | chr14 | q |
| 72 | AK7 | NM_152327 | chr14 | q |
| 72 | EVL | NM_016337 | chr14 | q |
| 72 | DEGS2 | NM_206918 | chr14 | q |
| 72 | ZNF839 | NM_018335 | chr14 | q |
| 72 | C14orf79 | NM_174891 | chr14 | q |
| 73 | C15orf23 | NM_001142762 | chr15 | q |
| 73 | C15orf23 | NM_033286 | chr15 | q |
| 73 | C15orf23 | NM_001142761 | chr15 | q |
| 73 | CASC5 | NM_170589 | chr15 | q |
| 73 | CASC5 | NM_144508 | chr15 | q |
| 73 | RAD51 | NM_002875 | chr15 | q |
| 73 | RAD51 | NM_133487 | chr15 | q |
| 73 | RAD51 | NM_001164270 | chr15 | q |
| 73 | RAD51 | NM_001164269 | chr15 | q |
| 73 | RHOV | NM_133639 | chr15 | q |
| 73 | INO80 | NM_017553 | chr15 | q |
| 73 | OIP5 | NM_007280 | chr15 | q |
| 73 | NUSAP1 | NM_001129897 | chr15 | q |
| 73 | NUSAP1 | NM_018454 | chr15 | q |
| 73 | NUSAP1 | NM_016359 | chr15 | q |
| 73 | ELL3 | NM_025165 | chr15 | q |
| 73 | SPATA5L1 | NM_024063 | chr15 | q |
| 73 | SPATA5L1 | NR_027635 | chr15 | q |
| 73 | SLC12A1 | NM_000338 | chr15 | q |
| 73 | SLC12A1 | NM_001184832 | chr15 | q |
| 73 | CEP152 | NM_001194998 | chr15 | q |
| 73 | CEP152 | NM_014985 | chr15 | q |
| 73 | SECISBP2L | NM_014701 | chr15 | q |
| 73 | SECISBP2L | NM_001193489 | chr15 | q |
| 73 | GALK2 | NM_002044 | chr15 | q |
| 73 | GALK2 | NM_001001556 | chr15 | q |
| 73 | GABPB1 | NM_016654 | chr15 | q |
| 73 | GABPB1 | NM_181427 | chr15 | q |
| 73 | GABPB1 | NM_005254 | chr15 | q |
| 73 | GABPB1 | NM_002041 | chr15 | q |
| 73 | GABPB1 | NM_016655 | chr15 | q |
| 73 | USP8 | NM_005154 | chr15 | q |
| 73 | USP8 | NM_001128611 | chr15 | q |
| 73 | USP8 | NM_001128610 | chr15 | q |
| 73 | TRPM7 | NM_017672 | chr15 | q |
| 73 | SPPL2A | NM_032802 | chr15 | q |
| 73 | AP4E1 | NM_007347 | chr15 | q |
| 73 | TNFAIP8L3 | NM_207381 | chr15 | q |
| 73 | CYP19A1 | NM_000103 | chr15 | q |
| 73 | CYP19A1 | NM_031226 | chr15 | q |
| 73 | TMOD2 | NM_014548 | chr15 | q |
| 73 | TMOD2 | NM_001142885 | chr15 | q |
| 73 | TMOD3 | NM_014547 | chr15 | q |
| 73 | MAPK6 | NM_002748 | chr15 | q |
| 73 | ARPP19 | NM_006628 | chr15 | q |
| 74 | GTSE1 | NM_016426 | chr22 | q |
| 74 | BRD1 | NM_014577 | chr22 | q |
| 74 | ZBED4 | NM_014838 | chr22 | q |
| 74 | CRELD2 | NM_024324 | chr22 | q |
| 74 | CRELD2 | NM_001135101 | chr22 | q |
| 74 | PIM3 | NM_001001852 | chr22 | q |
| 74 | TRABD | NM_025204 | chr22 | q |
| 74 | SELO | NM_031454 | chr22 | q |
| 75 | EVX1 | NM_001989 | chr7 | p |
| 75 | HIBADH | NM_152740 | chr7 | p |
| 75 | TAX1BP1 | NM_006024 | chr7 | p |
| 75 | TAX1BP1 | NM_001079864 | chr7 | p |
| 75 | PLEKHA8 | NM_001197026 | chr7 | p |
| 75 | PLEKHA8 | NM_032639 | chr7 | p |
| 75 | PLEKHA8 | NM_001197027 | chr7 | p |
| 75 | ZNRF2 | NM_147128 | chr7 | p |
| 75 | NOD1 | NM_006092 | chr7 | p |
| 75 | GGCT | NR_037669 | chr7 | p |
| 75 | GGCT | NM_001199815 | chr7 | p |
| 75 | GGCT | NM_001199817 | chr7 | p |
| 75 | GGCT | NM_001199816 | chr7 | p |
| 75 | GGCT | NM_024051 | chr7 | p |
| 75 | GARS | NM_002047 | chr7 | p |
| 75 | ANLN | NM_018685 | chr7 | p |
| 75 | VPS41 | NM_080631 | chr7 | p |
| 75 | VPS41 | NM_014396 | chr7 | p |
| 75 | CDK13 | NM_003718 | chr7 | p |
| 75 | CDK13 | NM_031267 | chr7 | p |
| 75 | PPIA | NM_021130 | chr7 | p |
| 75 | TBRG4 | NM_004749 | chr7 | p |
| 75 | TBRG4 | NM_199122 | chr7 | p |
| 75 | TBRG4 | NM_030900 | chr7 | p |
| 75 | CHCHD2 | NM_016139 | chr7 | p |

What is claimed is:

1. A method for identifying a string of genes that are associated with clinical outcome of a disease, comprising
measuring gene expression levels of a plurality of genes obtained from a sample from a patient having the disease;
normalizing the gene expression levels to obtain normalized gene expression levels;
associating the normalized gene expression levels with the clinical outcome;
identifying genes that significantly associate with the clinical outcome;
aligning the genes that significantly associate with the clinical outcome to a human chromosome physical map; and
identifying a string of genes that positively or negatively associate with the clinical outcome.

2. The method of claim 1, wherein a gene comprises exons.

3. The method of claim 1, wherein a gene comprises introns.

4. The method of claim 1, wherein a gene comprises exons and introns.

5. The method of any one of claims 1-4, wherein, the disease is selected from cancer, diabetes, inflammatory diseases, neurodegenerative diseases, and heart disease.

6. The method of any one of claims 1-5, wherein the clinical outcome is selected from recurrence of the disease, prognosis of disease, and prediction of response to a drug.

7. The method of any one of claims 1-6, wherein the disease is breast cancer and the clinical outcome is recurrence of breast cancer.

8. The method of any one of claims 1-7, wherein gene expression levels are measured by a method selected from a sequencing-based method, an array based method, an amplification-based method, or a proteomic-based method.

9. The method of claim 8, wherein the amplification based method is RT-PCR.

10. The method of claim 8, wherein the proteomic-based method is selected from Western blotting and mass spectrometry.

11. The method of any of claims 1-10, wherein the sample is a tissue sample.

12. The method of claim 11, wherein the tissue sample comprises a tumor.

13. The method of claim 12, wherein the tumor is a breast tumor.

14. The method of claim 13, wherein the breast tumor is ER-positive.

15. The method of any of claims 1-14, wherein the sample is a fresh, frozen, or a fixed, wax-embedded tissue sample.

16. The method of any one of claims 1-10, wherein the sample is a liquid sample.

17. The method of claim 16, wherein the liquid sample is blood, plasma, or urine.

18. The method of any of claims 1-17, wherein the string of genes is identified by a minimum number of genes.

19. The method of claim 18, wherein the string of genes is defined by at least 5 genes, at least 10 genes, at least 15 genes, at least 20 genes, at least 25 genes, at least 30 genes, at least 35 genes, at least 40 genes, at least 45 genes, or at least 50 genes.

20. The method of any one of claims 1-19, wherein the string of genes is defined by a physical boundary.

21. The method of claim 20, wherein the physical boundary is an end of a chromosome or an end of a chromosome arm.

22. The method of any one of claims 1-21, wherein the string of genes is defined by functional homogeneity.

23. The method of claim 22, wherein functional homogeneity is a role in controlling proliferative genes or a common signaling pathway.

24. The method of any one of claims 1-23, wherein the string of genes is defined by genes that co-express with one another.

25. The method of any of one claims 1-24, further comprising representing the string of genes as a metagene and creating univariate or multivariate models to predict a clinical outcome.

26. A method of creating a transcriptome profile map, comprising:
measuring gene expression levels of a plurality of genes obtained from a sample from a human;
normalizing the gene expression levels to obtain normalized gene expression levels;
standardizing the normalized gene expression levels; and
generating a graphic, wherein the X-axis represents chromosomal locus of the plurality of genes and the Y-axis represents the standardized, normalized gene expression levels, wherein the graphic is a transcriptome profile map.

27. The method of claim 26, further comprising examining a map density feature of the transcriptome profile map for a biomarker of a clinical outcome.

28. The method of claim 27, predicting the clinical outcome based on the map density feature of the transcriptome profile map.

29. The method of any one of claim 27 or 28, wherein the clinical outcome is sensitivity or resistance to a drug, progression from early cancer to a more advanced stage of cancer, disease recurrence, or survival.

30. The method of claim 27, wherein the clinical outcome is recurrence of breast cancer.

31. The method of any one of claims 27-30, wherein the map density feature is global dispersion of transcriptome z-scores or number of Y-axis map segments.

32. The method of claim 31, wherein the map segment is identified using circular binary segmentation, piecewise constant fitting programs, minimum z-score cutoffs, or minimum number of gene product species in a row on the same side of the Y-axis zero value.

33. The method of any one of claims 26-32, wherein the sample is obtained from a human a disease.

34. The method of any one of claims 26-32, wherein gene expression levels are measured by a method selected from a sequencing-based method, an array based method, an amplification-based method, or a proteomic-based method.

35. The method of claim 34, wherein the amplification based method is RT-PCR.

36. The method of claim 34, wherein the proteomic-based method is selected from Western blotting and mass spectrometry.

37. The method of any of claims 26-36, wherein the sample is a tissue sample.

38. The method of claim 37, wherein the tissue sample comprises a tumor.

39. The method of claim 38, wherein the tumor is a breast tumor.

40. The method of claim 39, wherein the breast tumor is ER-positive.

41. The method of any of claims 26-40, wherein the sample is a fresh, frozen, or a fixed, wax-embedded tissue sample.

42. The method of any one of claims 26-36, wherein the sample is a liquid sample.

43. The method of claim 42, wherein the liquid sample is blood, plasma, or urine.
